(19) United States
(12) Patent Application Publication
(10) Pub. No.: US 2025/0333732 A1
(43) Pub. Date: Oct. 30, 2025

(54) TREATMENT OF SOS2 RELATED DISEASES AND DISORDERS

(71) Applicants: Empirico Inc., San Diego, CA (US); Empirico Inc., San Diego, CA (US)

(72) Inventors: Omri GOTTESMAN, San Diego, CA (US); Shannon BRUSE, San Diego, CA (US); Brian CAJES, San Diego, CA (US); David LEWIS, Madison, WI (US); David JAKUBOSKY, San Diego, CA (US); Gregory MCINNES, San Diego, CA (US); David ROZEMA, Cross Plains, WI (US); John VEKICH, San Diego, CA (US)

(21) Appl. No.: 18/866,499

(22) PCT Filed: May 23, 2023
(86) PCT No.: PCT/US2023/067360

§ 371 Date: Nov. 15, 2024

Related U.S. Application Data
(60) Provisional application No. 63/344,836, filed on May 23, 2022.

(51) Int. Cl. C12N 15/113

(52) U.S. Cl. CPC C12N 15/113; C12N 2310/11; C12N 2310/14; C12N 2310/315; C12N 2310/321; C12N 2310/322; C12N 2310/3231; C12N 2310/344; C12N 2310/3515; C12N 2310/3517; C12N 2320/30

(57) ABSTRACT

Disclosed herein are compositions comprising an oligonucleotide that targets SOS Ras/Rho guanine nucleotide exchange factor 2 (SOS2). The oligonucleotide may include a small interfering RNA (siRNA) or an antisense oligonucleotide (ASO). Also provided herein are methods of treating conditions associated with SOS2 mutations that include providing an oligonucleotide that targets SOS2 to a subject.

### CROSS-REFERENCE

This application claims the benefit of U.S. Provisional Application No. 63/344,836, filed on May 23, 2022, which application is incorporated herein by reference.

### INCORPORATION BY REFERENCE OF SEQUENCE LISTING

The present application is being filed along with a Sequence Listing in electronic format. The Sequence Listing is provided as a file entitled 54462-741_601_SL_1.xml, created May 18, 2023, which is 9,932,318 bytes in size. The information in the electronic format of the Sequence Listing is incorporated by reference in its entirety.

### BACKGROUND

Indications such as chronic kidney disease, diabetic nephropathy, gout, hyperuricemia, hypertension, cerebrovascular disease, type 2 diabetes, metabolic syndrome, obesity, hyperlipidemia, hypertriglyceridemia, glaucoma, ocular hypertension, retinal diseases, age-related macular degeneration, choroidal neovascularization, geographic atrophy, diabetic retinopathy, non-alcoholic fatty liver disease, fibrotic liver disease, liver fibrosis, cirrhosis, or hair loss may affect a wide variety of persons. Improved therapeutics are needed.

### SUMMARY

In certain aspects, disclosed herein is a composition comprising an oligonucleotide that targets SOS2 and when administered to a subject in an effective amount increases an estimated glomerular filtration rate, or decreases a creatinine, blood urea nitrogen, proteinuria microalbuminuria measurement, or urine albumin creatinine ratio. In some embodiments, the estimated glomerular filtration rate is increased, or the creatinine, blood urea nitrogen, proteinuria, microalbuminuria measurement or urine albumin creatinine ratio is decreased, by about 10% or more, as compared to prior to administration.

In certain aspects, disclosed herein is a composition comprising an oligonucleotide that targets SOS2 and when administered to a subject in an effective amount decreases a blood urate measurement. In some embodiments, the blood urate measurement is decreased by about 10% or more, as compared to prior to administration.

In certain aspects, disclosed herein is a composition comprising an oligonucleotide that targets SOS2 and when administered to a subject in an effective amount decreases a systolic blood pressure measurement, a diastolic blood pressure measurement, a mean arterial pressure, or a pulse pressure. In some embodiments, the systolic blood pressure measurement, diastolic blood pressure measurement, mean arterial pressure, or pulse pressure is decreased by about 10% or more, as compared to prior to administration.

In certain aspects, disclosed herein is a composition comprising an oligonucleotide that targets SOS2 and when administered to a subject in an effective amount decreases an intraocular pressure measurement, cup-disc ratio, optic nerve cupping, RPE pigmentation and reflectivity, drusen, Macular hemorrhage, choroidal neovascularization, edema, microaneurysms, intraretinal hemorrhage, macular ischemia, neovascularization, vitreous hemorrhage, or traction retinal detachment or increases a RNFL thickness or retinal thickness. In some embodiments, the intraocular pressure measurement, cup-disc ratio, optic nerve cupping, RPE pigmentation and reflectivity, drusen, Macular hemorrhage, choroidal neovascularization, edema, microaneurysms, intraretinal hemorrhage, macular ischemia, neovascularization, vitreous hemorrhage, or traction retinal detachment is decreased or the RNFL thickness or retinal thickness is increased by about 10% or more, as compared to prior to administration.

In certain aspects, disclosed herein is a composition comprising an oligonucleotide that targets SOS2 and when administered to a subject in an effective amount decreases a body mass index (BMI) measurement, a body weight measurement, a waist circumference measurement, a hip circumference measurement, a waist-hip ratio (WHR), a body fat percentage measurement, a hemoglobin A1C measurement, a blood glucose measurement, a glucose tolerance measurement, an insulin sensitivity measurement, a blood triglyceride measurement, or a non-HDL cholesterol measurement. In some embodiments, the body mass index (BMI) measurement, the body weight measurement, the waist circumference measurement, the hip circumference measurement, the waist-hip ratio (WHR), the body fat percentage measurement, the hemoglobin A1C measurement, the blood glucose measurement, the glucose tolerance measurement, the insulin sensitivity measurement, the blood triglyceride measurement, or the non-HDL cholesterol measurement is decreased by about 10% or more, as compared to prior to administration.

In certain aspects, disclosed herein is a composition comprising an oligonucleotide that targets SOS2 and when administered to a subject in an effective amount decreases an alanine aminotransferase, aspartate aminotransferase, liver fat percentage measurement, liver fibrosis score, NAFLD activity score, or blood gamma-glutamyl transferase measurement. In some embodiments, the alanine aminotransferase, aspartate aminotransferase, liver fat percentage measurement, liver fibrosis score, NAFLD activity score, or blood gamma-glutamyl transferase measurement is decreased by about 10% or more, as compared to prior to administration. In some embodiments, the oligonucleotide comprises a small interfering RNA (siRNA) comprising a sense strand and an antisense strand. In some embodiments, the sense strand is 12-30 nucleosides in length. In some embodiments, the sense strand comprises the sequence of any one of SEQ ID NOs: 1-5490, or a nucleic acid sequence thereof having 1 or 2 nucleoside substitutions, additions, or deletions. In some embodiments, the sense strand comprises the sequence of any one of SEQ ID NOs: 1-5490. In some embodiments, the antisense strand is 12-30 nucleosides in length. In some embodiments, the antisense strand comprises the sequence of any one of SEQ ID NOs: 5491-10980, or a nucleic acid sequence thereof having 1 or 2 nucleoside substitutions, additions, or deletions. In some embodiments, the antisense strand comprises the sequence of any one of SEQ ID NOs: 5491-10980. In some embodiments, the sense or antisense strand comprises a sense or antisense sequence of an siRNA of any one of Tables 35-35, or a nucleic acid sequence thereof having 1 or 2 nucleoside substitutions, additions, or deletions. In some embodiments, the sense or antisense strand comprises a sense or antisense sequence of an siRNA of any one of Tables 15-25. In some embodiments, any one of the following is true with regard to the sense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′ fluoro modified purines, and all pyrimidines comprise 2′-O-methyl modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′ fluoro modified pyrimidines, and all purines comprise 2′-O-methyl modified purines. In some embodiments, any one of the following is true with regard to the antisense strand: all purines comprise 2′ fluoro modified purines, and all pyrimidines comprise a mixture of 2′ fluoro and 2′-O-methyl modified pyrimidines; all purines comprise 2′-O-methyl modified purines, and all pyrimidines comprise a mixture of 2′ fluoro and 2′-O-methyl modified pyrimidines; all purines comprise 2′-O-methyl modified purines, and all pyrimidines comprise 2′ fluoro modified pyrimidines; all pyrimidines comprise 2′ fluoro modified pyrimidines, and all purines comprise a mixture of 2′ fluoro and 2′-O-methyl modified purines; all pyrimidines comprise 2′-O-methyl modified pyrimidines, and all purines comprise a mixture of 2′ fluoro and 2′-O-methyl modified purines; or all pyrimidines comprise 2′-O-methyl modified pyrimidines, and all purines comprise 2′ fluoro modified purines.

In certain aspects, disclosed herein is a composition comprising an oligonucleotide that inhibits the expression of SOS2 wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 11253. In some embodiments, the oligonucleotide comprises an antisense oligonucleotide (ASO).

In certain aspects, disclosed herein is a composition comprising an oligonucleotide that inhibits the expression of SOS2 wherein the oligonucleotide comprises an ASO that is complementary to a nucleoside sequence comprising about 12-30 contiguous nucleosides of SEQ ID NO: 11253. In some embodiments, the ASO is 12-30 nucleosides in length.

In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. In some embodiments, the oligonucleotide comprises 2, 3, 4, 5, 6, 7, 8, 9, 10, 11, 12, 13, 14, 15, 16, 17, 18, 19, or 20 modified internucleoside linkages. In some embodiments, the oligonucleotide comprises a modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises one or more 2′fluoro modified nucleosides. In some embodiments, the modified nucleoside comprises a 2′ O-alkyl modified nucleoside. In some embodiments, the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the lipid comprises a 5′ hydrophobic moiety comprising any one of the following structures:

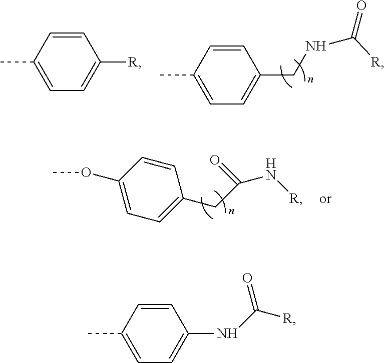

wherein the dotted line indicates a covalent connection to the end of the 5′ end of the sense strand, n is 1-3, and R is an alkyl group containing 4-18 carbons. In some embodiments, the oligonucleotide comprises 2, 3, 4, 5, 6, 7, 8, 9, 10, 11, 12, 13, 14, 15, 16, 17, 18, 19, 20, or 21 modified nucleosides. In some embodiments, the oligonucleotide comprises an N-acetylgalactosamine (GalNAc) ligand, an arginine-glycine-aspartic acid (RGD) peptide, or a cholesterol ligand. In some embodiments, the oligonucleotide comprises a GalNAc ligand. In some embodiments, the GalNac ligand comprises

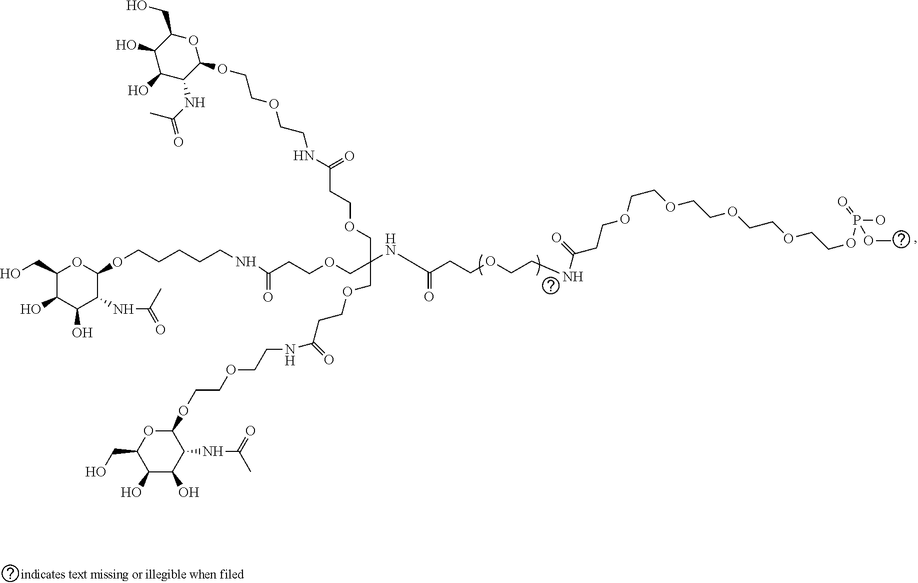

wherein n is 1 or 2, and J is the oligonucleotide.

In certain aspects, disclosed herein is a method of treating chronic kidney disease, diabetic nephropathy, gout, hyperuricemia, hypertension, cerebrovascular disease, type 2 diabetes, metabolic syndrome, obesity, hyperlipidemia, hypertriglyceridemia, glaucoma, ocular hypertension, retinal diseases, age-related macular degeneration, choroidal neovascularization, geographic atrophy, diabetic retinopathy, non-alcoholic fatty liver disease, fibrotic liver disease, liver fibrosis, cirrhosis, or hair loss in a subject in need thereof comprising administering to the subject a composition described herein.

In certain aspects, disclosed herein is a composition comprising an oligonucleotide that targets SOS2, wherein the oligonucleotide comprises a small interfering RNA (siRNA) comprising a sense strand and an antisense strand; and wherein the sense strand comprises the nucleoside sequence of any one of SEQ ID NOs: 1-5490 or a nucleic acid sequence thereof having 1 or 2 nucleoside substitutions, additions, or deletions; or wherein the antisense strand comprises the nucleoside sequence of any one of SEQ ID NOs: 5491-10980 or a nucleic acid sequence thereof having 1 or 2 nucleoside substitutions, additions, or deletions.

In certain aspects, disclosed herein is a composition comprising a compound represented by Formula (I) or (II):

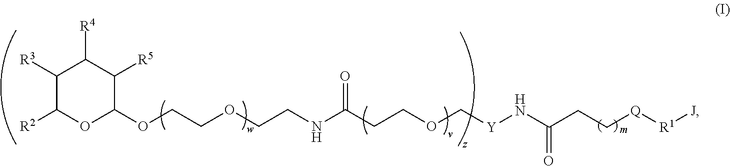

or

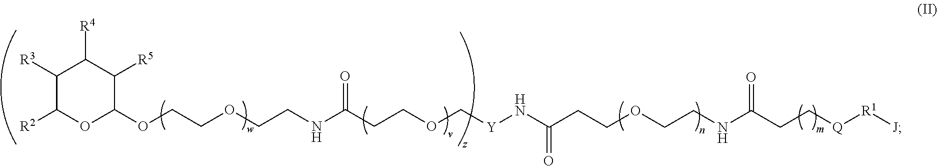

or a salt thereof, wherein J is an oligonucleotide targeting SOS comprising a small interfering RNA (siRNA) comprising a sense strand and an antisense strand; each w is independently selected from any value from 1 to 20; each v is independently selected from any value from 1 to 20; n is selected from any value from 1 to 20; m is selected from any value from 1 to 20; z is selected from any value from 1 to 3, wherein if z is 3, Y is C if z is 2, Y is CR⁶, or if z is 1, Y is C(R⁶)₂; Q is selected from: C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂; R¹ is a linker selected from: —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—; each R² is independently selected from: C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; R³ and R⁴ are each independently selected from: —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; each R⁵ is independently selected from: —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂; each R⁶ is independently selected from: hydrogen; halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; each R⁷ is independently selected from: hydrogen; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl.

A composition comprising an oligonucleotide that inhibits the expression of SOS2 wherein the oligonucleotide comprises an siRNA comprising a sense strand and an antisense strand, wherein the oligonucleotide comprises a 5′ hydrophobic moiety comprising any one of the following structures:

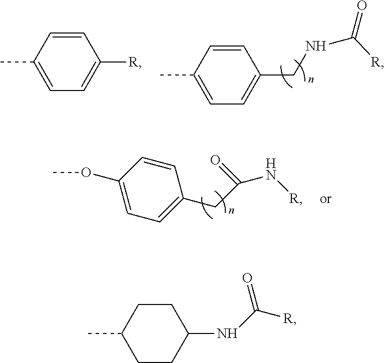

wherein the dotted line indicates a covalent connection to the end of the 5′ end of the sense strand, n is 1-3, and R is an alkyl group containing 4-18 carbons.

### DETAILED DESCRIPTION OF THE INVENTION

Large-scale human genetic data can improve the success rate of pharmaceutical discovery and development. A Genome Wide Association Study (GWAS) may detect associations between genetic variants and traits in a population sample. A GWAS may enable better understanding of the biology of disease, and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal genetic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, kidney, liver, adipocyte, or eye) and a relevant indication.

SOS Ras/Rho guanine nucleotide exchange factor 2 encodes son of sevenless homolog 2 (also “SOS2”), a regulatory protein that may be involved in the positive regulation of ras proteins. SOS2 may map to 14q21 within the human genome. SOS2 may activate RAC1. Mutations in SOS2 may relate to Noonan syndrome. Here it is shown that loss-of-function SOS2 variants resulted in protective associations. Therefore, inhibition of SOS2 may serve as a therapeutic for treatment of SOS2-related diseases and disorders. In particular, it is shown here that loss-of-function genetic variants of SOS2 may be protective for chronic kidney disease, diabetic nephropathy, gout, hyperuricemia, hypertension, cerebrovascular disease, type 2 diabetes, metabolic syndrome, obesity, hyperlipidemia, hypertriglyceridemia, glaucoma, ocular hypertension, retinal diseases, age-related macular degeneration, choroidal neovascularization, geographic atrophy, diabetic retinopathy, non-alcoholic fatty liver disease, fibrotic liver disease, liver fibrosis, cirrhosis, or hair loss (e.g. androgenetic alopecia). Therefore, inhibition of SOS2 may serve as a therapeutic for treatment of these indications.

Disclosed herein are compositions comprising an oligonucleotide that targets SOS2. The oligonucleotide may include a small interfering RNA (siRNA) or an antisense oligonucleotide (ASO). Also provided herein are methods of treating chronic kidney disease, diabetic nephropathy, gout, hyperuricemia, hypertension, cerebrovascular disease, type 2 diabetes, metabolic syndrome, obesity, hyperlipidemia, hypertriglyceridemia, glaucoma, ocular hypertension, retinal diseases, age-related macular degeneration, choroidal neovascularization, geographic atrophy, diabetic retinopathy, non-alcoholic fatty liver disease, fibrotic liver disease, liver fibrosis, cirrhosis, or hair loss (e.g. androgenetic alopecia) by providing an oligonucleotide that targets SOS2 to a subject in need thereof.

### I. COMPOSITIONS

Disclosed herein, in some embodiments, are compositions comprising an oligonucleotide. In some embodiments, the composition comprises an oligonucleotide that targets SOS Ras/Rho guanine nucleotide exchange factor 2 (SOS2). In some embodiments, the composition consists of an oligonucleotide that targets SOS2. In some embodiments, the oligonucleotide reduces SOS2 mRNA expression in the subject. In some embodiments, the oligonucleotide reduces son of sevenless homolog 2 (SOS2) protein expression in the subject. The oligonucleotide may include a small interfering RNA (siRNA) described herein. The oligonucleotide may include an antisense oligonucleotide (ASO) described herein. In some embodiments, a composition described herein is used in a method of treating a disorder in a subject in need thereof. Some embodiments relate to a composition comprising an oligonucleotide for use in a method of treating a disorder as described herein. Some embodiments relate to use of a composition comprising an oligonucleotide, in a method of treating a disorder as described herein.

Some embodiments include a composition comprising an oligonucleotide that targets SOS2 and when administered to a subject in an effective amount decreases SOS2 mRNA or SOS2 protein levels in a cell, fluid or tissue. In some embodiments, the composition comprises an oligonucleotide that targets SOS2 and when administered to a subject in an effective amount decreases SOS2 mRNA levels in a cell or tissue. In some embodiments, the cell is a liver cell (e.g. hepatocyte), kidney cell (e.g. podocyte), eye cell, or adipocyte. In some embodiments, the tissue is liver, kidney, eye, or adipose tissue. In some embodiments, the SOS2 mRNA levels are decreased by about 2.5% or more, about 5% or more, or about 7.5% or more, as compared to prior to administration. In some embodiments, the SOS2 mRNA levels are decreased by about 10% or more, as compared to prior to administration. In some embodiments, the SOS2 mRNA levels are decreased by about 20% or more, about 30% or more, about 40% or more, about 50% or more, about 60% or more, about 70% or more, about 80% or more, about 90% or more, about 95% or more, or about 100%, as compared to prior to administration. In some embodiments, the v mRNA levels are decreased by no more than about 2.5%, no more than about 5%, or no more than about 7.5%, as compared to prior to administration. In some embodiments, the SOS2 mRNA levels are decreased by no more than about 10%, as compared to prior to administration. In some embodiments, the SOS2 mRNA levels are decreased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the SOS2 mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets SOS2 and when administered to a subject in an effective amount decreases SOS2 protein levels in a cell or tissue. In some embodiments, the cell is a liver cell (e.g. hepatocyte), kidney cell (e.g. podocyte), eye cell, or adipocyte. In some embodiments, the tissue is liver, kidney, eye, or adipose tissue. In some embodiments, the SOS2 protein levels are decreased by about 2.5% or more, about 5% or more, or about 7.5% or more, as compared to prior to administration. In some embodiments, the SOS2 protein levels are decreased by about 10% or more, as compared to prior to administration. In some embodiments, the SOS2 protein levels are decreased by about 20% or more, about 30% or more, about 40% or more, about 50% or more, about 60% or more, about 70% or more, about 80% or more, about 90% or more, or about 100%, as compared to prior to administration. In some embodiments, the SOS2 protein levels are decreased by no more than about 2.5%, no more than about 5%, or no more than about 7.5%, as compared to prior to administration. In some embodiments, the SOS2 protein levels are decreased by no more than about 10%, as compared to prior to administration. In some embodiments, the SOS2 protein levels are decreased by no more than about 20%, no more than about 30%, no more than about 40%, no more than about 50%, no more than about 60%, no more than about 70%, no more than about 80%, no more than about 90%, about 95% or more, or no more than about 100%, as compared to prior to administration. In some embodiments, the SOS2 protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises an oligonucleotide that targets SOS2 and when administered to a subject in an effective amount decreases a kidney disease-related parameter. In some embodiments, the kidney disease comprises chronic kidney disease (CKD). In some embodiments, the kidney disease comprises diabetic nephropathy. The kidney disease-related parameter may include a blood creatinine measurement. The kidney disease-related parameter may include a blood urea nitrogen (BUN) measurement. The kidney disease-related parameter may include a BUN/creatinine measurement. The parameter may include a proteinuria measurement. The parameter may include a microalbuminuria measurement. The parameter may comprise a urine albumin creatinine ratio. In some embodiments, the kidney disease-related parameter is decreased by about 2.5% or more, about 5% or more, or about 7.5% or more, as compared to prior to administration. In some embodiments, the kidney disease-related parameter is decreased by about 10% or more, as compared to prior to administration. In some embodiments, the kidney disease-related parameter is decreased by about 20% or more, about 30% or more, about 40% or more, about 50% or more, about 60% or more, about 70% or more, about 80% or more, or about 90% or more, as compared to prior to administration. In some embodiments, the kidney disease-related parameter is decreased by no more than about 2.5%, no more than about 5%, or no more than about 7.5%, as compared to prior to administration. In some embodiments, the kidney disease-related parameter is decreased by no more than about 10%, as compared to prior to administration. In some embodiments, the kidney disease-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the kidney disease-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount increases a kidney disease-related parameter. The kidney disease-related parameter may include a glomerular filtration rate (GFR). The kidney disease-related parameter may include an estimated glomerular filtration rate (eGFR). In some embodiments, the kidney disease-related parameter is increased by about 2.5% or more, about 5% or more, or about 7.5% or more, as compared to prior to administration. In some embodiments, the kidney disease-related parameter is increased by about 10% or more, as compared to prior to administration. In some embodiments, the kidney disease-related parameter is increased by about 20% or more, about 30% or more, about 40% or more, about 50% or more, about 60% or more, about 70% or more, about 80% or more, about 90% or more, or about 100% or more, as compared to prior to administration. In some embodiments, the kidney disease-related parameter is increased by about 200% or more, about 300% or more, about 400% or more, about 500% or more, about 600% or more, about 700% or more, about 800% or more, about 900% or more, or about 1000% or more, as compared to prior to administration. In some embodiments, the kidney disease-related parameter is increased by no more than about 2.5%, no more than about 5%, or no more than about 7.5%, as compared to prior to administration. In some embodiments, the kidney disease-related parameter is increased by no more than about 10%, as compared to prior to administration. In some embodiments, the kidney disease-related parameter is increased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the kidney disease-related parameter is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the kidney disease-related parameter is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, orby a range defined by any of the two aforementioned percentages.

In some embodiments, the composition comprises an oligonucleotide that targets SOS2 and when administered to a subject in an effective amount decreases a gout-related or hyperuricemia-related parameter. The gout-related or hyperuricemia-related parameter may comprise a gout-related parameter. The gout-related or hyperuricemia-related parameter may comprise a hyperuricemia-related parameter. The gout-related or hyperuricemia-related parameter may include a blood urate measurement. In some embodiments, the gout-related or hyperuricemia-related parameter is decreased by about 2.5% or more, about 5% or more, or about 7.5% or more, as compared to prior to administration. In some embodiments, the gout-related or hyperuricemia-related parameter is decreased by about 10% or more, as compared to prior to administration. In some embodiments, the gout-related or hyperuricemia-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the gout-related or hyperuricemia-related parameter is decreased by no more than about 2.5%, no more than about 5%, or no more than about 7.5%, as compared to prior to administration. In some embodiments, the gout-related or hyperuricemia-related parameter is decreased by no more than about 10%, as compared to prior to administration. In some embodiments, the gout-related or hyperuricemia-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the gout-related or hyperuricemia-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a cerebrovascular disease-related parameter. The cerebrovascular disease-related parameter may include a hypertension-related parameter. The hypertension-related parameter may include a systolic blood pressure measurement. The hypertension-related parameter may include a diastolic blood pressure measurement. The hypertension-related parameter may include a mean arterial pressure measurement. The hypertension-related parameter may include a pulse pressure measurement. In some embodiments, the hypertension-related parameter is decreased by about 2.5% or more, about 5% or more, or about 7.5% or more, as compared to prior to administration. In some embodiments, the hypertension-related parameter is decreased by about 10% or more, as compared to prior to administration. In some embodiments, the hypertension-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the hypertension-related parameter is decreased by no more than about 2.5%, no more than about 5%, or no more than about 7.5%, as compared to prior to administration. In some embodiments, the hypertension-related parameter is decreased by no more than about 10%, as compared to prior to administration. In some embodiments, the hypertension-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the hypertension-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a glaucoma-related parameter such as an adverse glaucoma-related parameter. The glaucoma-related parameter may include a intraocular pressure measurement. The glaucoma-related parameter may include a cup-disc ratio. The glaucoma related parameter may include optic nerve head cupping. In some embodiments, the glaucoma-related parameter is decreased by about 2.5% or more, about 5% or more, or about 7.5% or more, as compared to prior to administration. In some embodiments, the glaucoma-related parameter is decreased by about 10% or more, as compared to prior to administration. In some embodiments, the glaucoma-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the glaucoma-related parameter is decreased by no more than about 2.5%, no more than about 5%, or no more than about 7.5%, as compared to prior to administration. In some embodiments, the glaucoma-related parameter is decreased by no more than about 10%, as compared to prior to administration. In some embodiments, the glaucoma-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the glaucoma-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount increases a glaucoma-related parameter such as a protective or beneficial glaucoma-related parameter. The glaucoma-related parameter may include a retinal nerve fiber layer (RNFL) thickness. The glaucoma related parameter may include optic nerve head cupping. In some embodiments, the glaucoma-related parameter is increased by about 2.5% or more, about 5% or more, or about 7.5% or more, as compared to prior to administration. In some embodiments, the glaucoma-related parameter is increased by about 10% or more, as compared to prior to administration. In some embodiments, the glaucoma-related parameter is increased by about 20% or more, about 30% or more, about 40% or more, about 50% or more, about 60% or more, about 70% or more, about 80% or more, about 90% or more, or about 95%, as compared to prior to administration. In some embodiments, the glaucoma-related parameter is increased by about 200% or more, about 300% or more, about 400% or more, about 500% or more, about 600% or more, about 700% or more, about 800% or more, about 900% or more, or about 1000% or more, as compared to prior to administration. In some embodiments, the glaucoma-related parameter is increased by no more than about 2.5%, no more than about 5%, or no more than about 7.5%, as compared to prior to administration. In some embodiments, the glaucoma-related parameter is increased by no more than about 10%, as compared to prior to administration. In some embodiments, the glaucoma-related parameter is increased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the glaucoma-related parameter is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the glaucoma-related parameter is increased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages. In some embodiments, the glaucoma-related parameter is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets SOS2 and when administered to a subject in an effective amount decreases a macular degeneration/diabetic retinopathy-related parameter such as an adverse macular degeneration/diabetic retinopathy-related parameter. The macular degeneration/diabetic retinopathy-related parameter may comprise a macular degeneration-related parameter. The macular degeneration/diabetic retinopathy-related parameter may comprise a diabetic retinopathy-related parameter. The macular degeneration/diabetic retinopathy-related parameter may include a RPE pigmentation and reflectivity measurement. The macular degeneration/diabetic retinopathy-related parameter may include a drusen measurement. The macular degeneration/diabetic retinopathy-related parameter may include a macular hemorrhage measurement. The macular degeneration/diabetic retinopathy-related parameter may include a choroidal neovascularization measurement. The macular degeneration/diabetic retinopathy-related parameter may include a edema measurement. The macular degeneration/diabetic retinopathy-related parameter may include a microaneurysm measurement. The macular degeneration/diabetic retinopathy-related parameter may include a intraretinal hemorrhage measurement. The macular degeneration/diabetic retinopathy-related parameter may include a macular ischemia measurement. The macular degeneration/diabetic retinopathy-related parameter may include a neovascularization measurement. The macular degeneration/diabetic retinopathy-related parameter may include a vitreous hemorrhage measurement. The macular degeneration/diabetic retinopathy-related parameter may include a traction retinal detachment measurement. In some embodiments, the macular degeneration/diabetic retinopathy-related parameter is decreased by about 2.5% or more, about 5% or more, or about 7.5% or more, as compared to prior to administration. In some embodiments, the macular degeneration/diabetic retinopathy-related parameter is decreased by about 10% or more, as compared to prior to administration. In some embodiments, the a macular degeneration/diabetic retinopathy-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the a macular degeneration/diabetic retinopathy-related parameter is decreased by no more than about 2.5%, no more than about 5%, or no more than about 7.5%, as compared to prior to administration. In some embodiments, the macular degeneration/diabetic retinopathy-related parameter is decreased by no more than about 10%, as compared to prior to administration. In some embodiments, the macular degeneration/diabetic retinopathy-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the a macular degeneration/diabetic retinopathy-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount increases a macular degeneration/diabetic retinopathy-related parameter such as a protective or beneficial macular degeneration/diabetic retinopathy-related parameter. The macular degeneration/diabetic retinopathy-related parameter may include a retinal thickness measurement. In some embodiments, the macular degeneration/diabetic retinopathy-related parameter is increased by about 2.5% or more, about 5% or more, or about 7.5% or more, as compared to prior to administration. In some embodiments, the macular degeneration/diabetic retinopathy-related parameter is increased by about 10% or more, as compared to prior to administration. In some embodiments, the a macular degeneration/diabetic retinopathy-related parameter is increased by about 20% or more, about 30% or more, about 40% or more, about 50% or more, about 60% or more, about 70% or more, about 80% or more, about 90% or more, or about 95%, as compared to prior to administration. In some embodiments, the a macular degeneration/diabetic retinopathy-related parameter is increased by no more than about 2.5%, no more than about 5%, or no more than about 7.5%, as compared to prior to administration. In some embodiments, the macular degeneration/diabetic retinopathy-related parameter is increased by no more than about 10%, as compared to prior to administration. In some embodiments, the macular degeneration/diabetic retinopathy-related parameter is increased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the macular degeneration/diabetic retinopathy-related parameter is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the a macular degeneration/diabetic retinopathy-related parameter is increased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages. In some embodiments, the macular degeneration/diabetic retinopathy-related parameter is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

In some embodiments, the composition comprises an oligonucleotide that targets SOS2 and when administered to a subject in an effective amount decreases a metabolic disorder-related parameter. In some embodiments, the metabolic disorder comprises obesity. In some embodiments, the metabolic disorder comprises hyperlipidemia. In some embodiments, the metabolic disorder comprises hypertriglyceridemia. In some embodiments, the metabolic disorder comprises metabolic syndrome. In some embodiments, the metabolic disorder comprises diabetes. In some embodiments, the diabetes comprises type II diabetes. The metabolic disorder-related parameter may include a hemoglobin A1C measurement. The metabolic disorder-related parameter may include a body mass index (BMI) measurement. The metabolic disorder-related parameter may include a body weight measurement. The metabolic disorder-related parameter may include a waist circumference measurement. The metabolic disorder-related parameter may include a hip circumference measurement. The metabolic disorder-related parameter may comprise a waist-hip ratio (WHR). The metabolic disorder-related parameter may comprise a body fat percentage. The metabolic disorder-related parameter may comprise a blood glucose measurement. The metabolic disorder-related parameter may comprise a glucose tolerance measurement. The metabolic disorder-related parameter may comprise a insulin sensitivity measurement. The metabolic disorder-related parameter may comprise a blood triglyceride measurement. The metabolic disorder-related parameter may comprise a non-HDL cholesterol measurement. In some embodiments, the metabolic disorder-related parameter is decreased by about 2.5% or more, about 5% or more, or about 7.5% or more, as compared to prior to administration. In some embodiments, the metabolic disorder-related parameter is decreased by about 10% or more, as compared to prior to administration. In some embodiments, the metabolic disorder-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the metabolic disorder-related parameter is decreased by no more than about 2.5%, no more than about 5%, or no more than about 7.5%, as compared to prior to administration. In some embodiments, the metabolic disorder-related parameter is decreased by no more than about 10%, as compared to prior to administration. In some embodiments, the metabolic disorder-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the metabolic disorder-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a liver disease-related parameter. In some embodiments, the liver disease comprises fibrotic liver disease. In some embodiments, the liver disease comprises liver fibrosis. In some embodiments, the liver disease comprises cirrhosis. In some embodiments, the liver disease comprises non-alcoholic fatty liver disease (NAFLD). The liver disease-related parameter may include an aspartate aminotransferase (AST) measurement. The liver disease-related parameter may include an alanine aminotransferase (ALT) measurement. The liver disease-related parameter may include an AST/ALT ratio. The liver disease-related parameter may include a liver fat percentage measurement. The liver disease-related parameter may include a liver fibrosis score. The liver disease-related parameter may include a NAFLD activity score. The liver disease-related parameter may include a blood gamma-glutamyl transferase (GGT) measurement. In some embodiments, the liver disease-related parameter is decreased by about 2.5% or more, about 5% or more, or about 7.5% or more, as compared to prior to administration. In some embodiments, the liver disease-related parameter is decreased by about 10% or more, as compared to prior to administration. In some embodiments, the liver disease-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the liver disease-related parameter is decreased by no more than about 2.5%, no more than about 5%, or no more than about 7.5%, as compared to prior to administration. In some embodiments, the liver disease-related parameter is decreased by no more than about 10%, as compared to prior to administration. In some embodiments, the liver disease-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the liver disease-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

In some embodiments, the composition comprises an oligonucleotide that targets SOS2 and when administered to a subject in an effective amount decreases a hair loss-related parameter. In some embodiments, the hair loss comprises androgenetic alopecia. The hair loss-related parameter may include a hair count measurement. The hair loss-related parameter may include a hair thickness measurement. The hair loss-related parameter may include a hair density measurement. In some embodiments, the hair loss-related parameter is decreased by about 2.5% or more, about 5% or more, or about 7.5% or more, as compared to prior to administration. In some embodiments, the hair loss-related parameter is decreased by about 10% or more, as compared to prior to administration. In some embodiments, the hair loss-related parameter is decreased by about 20% or more, about 30% or more, about 40% or more, about 50% or more, about 60% or more, about 70% or more, about 80% or more, about 90% or more, or about 95%, as compared to prior to administration. In some embodiments, the hair loss-related parameter is decreased by no more than about 2.5%, no more than about 5%, or no more than about 7.5%, as compared to prior to administration. In some embodiments, the hair loss-related parameter is decreased by no more than about 10%, as compared to prior to administration. In some embodiments, the hair loss-related parameter is decreased by no more than about 20%, no more than about 30%, no more than about 40%, no more than about 50%, no more than about 60%, no more than about 70%, no more than about 80%, no more than about 90%, or no more than about 95%, as compared to prior to administration. In some embodiments, the hair loss-related parameter is decreased by 2.5%, 5%, 7.5%, 10%, 15%, 20%, 25%, 30%, 35%, 40%, 45%, 50%, 55%, 60%, 65%, 70%, 75%, 80%, 85%, 90%, or 95%, or by a range defined by any of the two aforementioned percentages.

### A. siRNAs

In some embodiments, the composition comprises an oligonucleotide that targets SOS Ras/Rho guanine nucleotide exchange factor 2 (SOS2), wherein the oligonucleotide comprises a small interfering RNA (siRNA). In some embodiments, the composition comprises an oligonucleotide that targets SOS2, wherein the oligonucleotide comprises a small interfering RNA (siRNA) comprising a sense strand and an antisense strand.

In some embodiments, the composition comprises an oligonucleotide that inhibits the expression of SOS2, wherein the oligonucleotide comprises an siRNA comprising a sense strand and an antisense strand, wherein the sense strand is 14-30 nucleosides in length. In some embodiments, the composition comprises a sense strange that is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the composition comprises an antisense strand is 14-30 nucleosides in length. In some embodiments, the composition comprises an antisense strange that is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers.

In some embodiments, the composition comprises an oligonucleotide that inhibits the expression of SOS2, wherein the oligonucleotide comprises an siRNA comprising a sense strand and an antisense strand, each strand is independently about 14-30 nucleosides in length, and at least one of the sense strand and the antisense strand comprises a nucleoside sequence comprising about 14-30 contiguous nucleosides of a full-length human SOS2 mRNA sequence such as SEQ ID NO: 11253. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 11253.

In some embodiments, the composition comprises an oligonucleotide that inhibits the expression of SOS2, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of SOS2, wherein the oligonucleotide comprises an siRNA comprising a sense strand and an antisense strand, wherein the siRNA binds with a 19mer in a human SOS2 mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human SOS2 mRNA.

In some embodiments, the composition comprises an oligonucleotide that inhibits the expression of SOS2, wherein the oligonucleotide comprises an siRNA comprising a sense strand and an antisense strand, wherein the siRNA binds with a 17mer in a non-human primate SOS2 mRNA. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate SOS2 mRNA.

In some embodiments, the composition comprises an oligonucleotide that inhibits the expression of SOS2, wherein the oligonucleotide comprises an siRNA comprising a sense strand and an antisense strand, wherein the siRNA binds with a 19mer in a human SOS2 mRNA, or a combination thereof. In some embodiments, the siRNA binds with a 12mer, a 13mer, a 14mer, a 15mer, a 16mer, a 17mer, and 18mer, a 19mer, a 20mer, a 21mer, a 22mer, a 23mer, a 24mer, or a 25mer in a human SOS2 mRNA.

In some embodiments, the composition comprises an oligonucleotide that inhibits the expression of SOS2, wherein the oligonucleotide comprises an siRNA comprising a sense strand and an antisense strand, wherein the siRNA binds with a human SOS2 mRNA and less than or equal to 20 human off-targets, with no more than 2 mismatches in the antisense strand. In some embodiments, the siRNA binds with a human SOS2 mRNA and less than or equal to 10 human off-targets, with no more than 2 mismatches in the antisense strand. In some embodiments, the siRNA binds with a human SOS2 mRNA and less than or equal to 30 human off-targets, with no more than 2 mismatches in the antisense strand. In some embodiments, the siRNA binds with a human SOS2 mRNA and less than or equal to 40 human off-targets, with no more than 2 mismatches in the antisense strand. In some embodiments, the siRNA binds with a human SOS2 mRNA and less than or equal to 50 human off-targets, with no more than 2 mismatches in the antisense strand. In some embodiments, the siRNA binds with a human SOS2 mRNA and less than or equal to 10 human off-targets, with no more than 3 mismatches in the antisense strand. In some embodiments, the siRNA binds with a human SOS2 mRNA and less than or equal to 20 human off-targets, with no more than 3 mismatches in the antisense strand. In some embodiments, the siRNA binds with a human SOS2 mRNA and less than or equal to 30 human off-targets, with no more than 3 mismatches in the antisense strand. In some embodiments, the siRNA binds with a human SOS2 mRNA and less than or equal to 40 human off-targets, with no more than 3 mismatches in the antisense strand. In some embodiments, the siRNA binds with a human SOS2 mRNA and less than or equal to 50 human off-targets, with no more than 3 mismatches in the antisense strand.

In some embodiments, the composition comprises an oligonucleotide that inhibits the expression of SOS2, wherein the oligonucleotide comprises an siRNA comprising a sense strand and an antisense strand, siRNA binds with a human SOS2 mRNA target site that does not harbor an SNP, with a minor allele frequency (MAF) greater or equal to 1% (pos. 2-18). In some embodiments, the MAF is greater or equal to about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In some embodiments, the siRNA comprises a sense strand having a sequence in accordance with any of SEQ ID NOs: 1-5490. In some embodiments, the sense strand sequence comprises or consists of sequence at least 75% identical to any one of SEQ ID NOs: 1-5490, at least 80% identical to any one of SEQ ID NOs: 1-5490, at least 85% identical to of any one of SEQ ID NOs: 1-5490, at least 90% identical to any one of SEQ ID NOs: 1-5490, or at least 95% identical to any one of SEQ ID NOs: 1-5490. In some embodiments, the sense strand sequence comprises or consists of the sequence of any one of SEQ ID NOs: 1-5490, or a sense strand sequence thereof having 1, 2, 3, or 4 nucleoside substitutions, additions, or deletions. In some embodiments, the sense strand sequence comprises or consists of the sequence of any one of SEQ ID NOs: 1-5490, or a sense strand sequence thereof having 1 or 2 nucleoside substitutions, additions, or deletions. In some embodiments, the sense strand sequence comprises or consists of a sequence 100% identical to SEQ ID NOs: 1-5490. The sense strand may comprise a modification pattern described herein. The sense strand may comprise a lipid moiety. The sense strand may comprise a GalNAc moiety.

In some embodiments, the siRNA comprises an antisense strand having a sequence in accordance with any of SEQ ID NOs: 5491-10980. In some embodiments, the antisense strand sequence comprises or consists of sequence at least 75% identical to any one of SEQ ID NOs: 5491-10980, at least 80% identical to any one of SEQ ID NOs: 5491-10980, at least 85% identical to of any one of SEQ ID NOs: 5491-10980, at least 90% identical to any one of SEQ ID NOs: 5491-10980, or at least 95% identical to any one of SEQ ID NOs: 5491-10980. In some embodiments, the antisense strand sequence comprises or consists of the sequence of any one of SEQ ID NOs: 5491-10980, or an antisense strand sequence thereof having 1, 2, 3, or 4 nucleoside substitutions, additions, or deletions. In some embodiments, the antisense strand sequence comprises or consists of the sequence of any one of SEQ ID NOs: 5491-10980, or an antisense strand sequence thereof having 1 or 2 nucleoside substitutions, additions, or deletions. In some embodiments, the antisense strand sequence comprises or consists of a sequence 100% identical to SEQ ID NOs: 5491-10980. The antisense strand may comprise a modification pattern described herein.

In some embodiments, the siRNA comprises a sense strand or antisense strand having a sequence in accordance with the sense strand or antisense strand sequence of an siRNA of subset A. In some embodiments, the sense strand or antisense strand comprises a sequence at least 75% identical, at least 80% identical, at least 85% identical, at least 90% identical, or at least 95% identical, to a sense strand or antisense strand sequence of subset A. In some embodiments, the sense strand or antisense strand comprises a sequence of a sense or antisense strand of subset A, or a sequence thereof having 3 or 4 nucleoside substitutions, additions, or deletions. In some embodiments, the sense strand or antisense strand comprises a sequence of a sense or antisense strand of subset A, or a sequence thereof having 1 or 2 nucleoside substitutions, additions, or deletions. In some embodiments, the sense strand or antisense sequence comprises or consists of a sequence 100% identical to a sense strand or antisense strand sequence of subset A. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset B. In some embodiments, the sense strand or antisense strand comprises a sequence at least 75% identical, at least 80% identical, at least 85% identical, at least 90% identical, or at least 95% identical, to a sense strand or antisense strand sequence of subset B. In some embodiments, the sense strand or antisense strand comprises a sequence of a sense or antisense strand of subset B, or a sequence thereof having 3 or 4 nucleoside substitutions, additions, or deletions. In some embodiments, the sense strand or antisense strand comprises a sequence of a sense or antisense strand of subset B, or a sequence thereof having 1 or 2 nucleoside substitutions, additions, or deletions. In some embodiments, the sense strand or antisense sequence comprises or consists of a sequence 100% identical to a sense strand or antisense strand sequence of subset B. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset C. In some embodiments, the sense strand or antisense strand comprises a sequence at least 75% identical, at least 80% identical, at least 85% identical, at least 90% identical, or at least 95% identical, to a sense strand or antisense strand sequence of subset C. In some embodiments, the sense strand or antisense strand comprises a sequence of a sense or antisense strand of subset C, or a sequence thereof having 3 or 4 nucleoside substitutions, additions, or deletions. In some embodiments, the sense strand or antisense strand comprises a sequence of a sense or antisense strand of subset C, or a sequence thereof having 1 or 2 nucleoside substitutions, additions, or deletions. In some embodiments, the sense strand or antisense sequence comprises or consists of a sequence 100% identical to a sense strand or antisense strand sequence of subset C. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset D. In some embodiments, the sense strand or antisense strand comprises a sequence at least 750% identical, at least 80% identical, at least 85% identical, at least 90% identical, or at least 95% identical, to a sense strand or antisense strand sequence of subset D. In some embodiments, the sense strand or antisense strand comprises a sequence of a sense or antisense strand of subset D, or a sequence thereof having 3 or 4 nucleoside substitutions, additions, or deletions. In some embodiments, the sense strand or antisense strand comprises a sequence of a sense or antisense strand of subset D, or a sequence thereof having 1 or 2 nucleoside substitutions, additions, or deletions. In some embodiments, the sense strand or antisense sequence comprises or consists of a sequence 100% identical to a sense strand or antisense strand sequence of subset D. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset E. In some embodiments, the sense strand or antisense strand comprises a sequence at least 75% identical, at least 80% identical, at least 85% identical, at least 90% identical, or at least 95% identical, to a sense strand or antisense strand sequence of subset E. In some embodiments, the sense strand or antisense strand comprises a sequence of a sense or antisense strand of subset E, or a sequence thereof having 3 or 4 nucleoside substitutions, additions, or deletions. In some embodiments, the sense strand or antisense strand comprises a sequence of a sense or antisense strand of subset E, or a sequence thereof having 1 or 2 nucleoside substitutions, additions, or deletions. In some embodiments, the sense strand or antisense sequence comprises or consists of a sequence 100% identical to a sense strand or antisense strand sequence of subset E. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset F. In some embodiments, the sense strand or antisense strand comprises a sequence at least 75% identical, at least 80% identical, at least 85% identical, at least 90% identical, or at least 95% identical, to a sense strand or antisense strand sequence of subset F. In some embodiments, the sense strand or antisense strand comprises a sequence of a sense or antisense strand of subset F, or a sequence thereof having 3 or 4 nucleoside substitutions, additions, or deletions. In some embodiments, the sense strand or antisense strand comprises a sequence of a sense or antisense strand of subset F, or a sequence thereof having 1 or 2 nucleoside substitutions, additions, or deletions. In some embodiments, the sense strand or antisense sequence comprises or consists of a sequence 100% identical to a sense strand or antisense strand sequence of subset F. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset G. In some embodiments, the sense strand or antisense strand comprises a sequence at least 75% identical, at least 80% identical, at least 85% identical, at least 90% identical, or at least 95% identical, to a sense strand or antisense strand sequence of subset G. In some embodiments, the sense strand or antisense strand comprises a sequence of a sense or antisense strand of subset G, or a sequence thereof having 3 or 4 nucleoside substitutions, additions, or deletions. In some embodiments, the sense strand or antisense strand comprises a sequence of a sense or antisense strand of subset G, or a sequence thereof having 1 or 2 nucleoside substitutions, additions, or deletions. In some embodiments, the sense strand or antisense sequence comprises or consists of a sequence 100% identical to a sense strand or antisense strand sequence of subset G. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset H. In some embodiments, the sense strand or antisense strand comprises a sequence at least 75% identical, at least 80% identical, at least 85% identical, at least 90% identical, or at least 95% identical, to a sense strand or antisense strand sequence of subset H. In some embodiments, the sense strand or antisense strand comprises a sequence of a sense or antisense strand of subset H, or a sequence thereof having 3 or 4 nucleoside substitutions, additions, or deletions. In some embodiments, the sense strand or antisense strand comprises a sequence of a sense or antisense strand of subset H, or a sequence thereof having 1 or 2 nucleoside substitutions, additions, or deletions. In some embodiments, the sense strand or antisense sequence comprises or consists of a sequence 100% identical to a sense strand or antisense strand sequence of subset H. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset I. In some embodiments, the sense strand or antisense strand comprises a sequence at least 750% identical, at least 80% identical, at least 85% identical, at least 90% identical, or at least 95% identical, to a sense strand or antisense strand sequence of subset I. In some embodiments, the sense strand or antisense strand comprises a sequence of a sense or antisense strand of subset I, or a sequence thereof having 3 or 4 nucleoside substitutions, additions, or deletions. In some embodiments, the sense strand or antisense strand comprises a sequence of a sense or antisense strand of subset I, or a sequence thereof having 1 or 2 nucleoside substitutions, additions, or deletions. In some embodiments, the sense strand or antisense sequence comprises or consists of a sequence 100% identical to a sense strand or antisense strand sequence of subset I. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset J. In some embodiments, the sense strand or antisense strand comprises a sequence at least 75% identical, at least 80% identical, at least 85% identical, at least 90% identical, or at least 95% identical, to a sense strand or antisense strand sequence of subset J. In some embodiments, the sense strand or antisense strand comprises a sequence of a sense or antisense strand of subset J, or a sequence thereof having 3 or 4 nucleoside substitutions, additions, or deletions. In some embodiments, the sense strand or antisense strand comprises a sequence of a sense or antisense strand of subset J, or a sequence thereof having 1 or 2 nucleoside substitutions, additions, or deletions. In some embodiments, the sense strand or antisense sequence comprises or consists of a sequence 100% identical to a sense strand or antisense strand sequence of subset J. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA of subset K. In some embodiments, the sense strand or antisense strand comprises a sequence at least 75% identical, at least 80% identical, at least 85% identical, at least 90% identical, or at least 95% identical, to a sense strand or antisense strand sequence of subset K. In some embodiments, the sense strand or antisense strand comprises a sequence of a sense or antisense strand of subset K, or a sequence thereof having 3 or 4 nucleoside substitutions, additions, or deletions. In some embodiments, the sense strand or antisense strand comprises a sequence of a sense or antisense strand of subset K, or a sequence thereof having 1 or 2 nucleoside substitutions, additions, or deletions. In some embodiments, the sense strand or antisense sequence comprises or consists of a sequence 100% identical to a sense strand or antisense strand sequence of subset K. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA in Table 28. In some embodiments, the sense strand or antisense strand comprises a sequence at least 750% identical, at least 80% identical, at least 85% identical, at least 90% identical, or at least 95% identical, to a sense strand or antisense strand sequence in Table 28. In some embodiments, the sense strand or antisense strand comprises a sequence of a sense or antisense strand in Table 28, or a sequence thereof having 3 or 4 nucleoside substitutions, additions, or deletions. In some embodiments, the sense strand or antisense strand comprises a sequence of a sense or antisense strand in Table 28, or a sequence thereof having 1 or 2 nucleoside substitutions, additions, or deletions. In some embodiments, the sense strand or antisense sequence comprises or consists of a sequence 100% identical to a sense strand or antisense strand sequence in Table 28. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA in Table 32. In some embodiments, the sense strand or antisense strand comprises a sequence at least 75% identical, at least 80% identical, at least 85% identical, at least 90% identical, or at least 95% identical, to a sense strand or antisense strand sequence in Table 32. In some embodiments, the sense strand or antisense strand comprises a sequence of a sense or antisense strand in Table 32, or a sequence thereof having 3 or 4 nucleoside substitutions, additions, or deletions. In some embodiments, the sense strand or antisense strand comprises a sequence of a sense or antisense strand in Table 32, or a sequence thereof having 1 or 2 nucleoside substitutions, additions, or deletions. In some embodiments, the sense strand or antisense sequence comprises or consists of a sequence 100% identical to a sense strand or antisense strand sequence in Table 32. The sense strand or antisense strand may comprise any modifications described herein. The sense strand or antisense strand may comprise a lipid moiety or a GalNAc moiety.

In some embodiments, the siRNA comprises a sense strand having a sequence in accordance with any of SEQ ID NOs: 743, 744, 1036, 1056, 1178, 1521, 2148, 2151, 2152, 2158, 2619, 2882, 2883, 2884, 3042, 3305, 3969, 4216, 4305 or 4309. In some embodiments, the sense strand sequence comprises or consists of sequence at least 75% identical to any one of SEQ ID NOs: 743, 744, 1036, 1056, 1178, 1521, 2148, 2151, 2152, 2158, 2619, 2882, 2883, 2884, 3042, 3305, 3969, 4216, 4305 or 4309, at least 80% identical to any one of SEQ ID NOs: 743, 744, 1036, 1056, 1178, 1521, 2148, 2151, 2152, 2158, 2619, 2882, 2883, 2884, 3042, 3305, 3969, 4216, 4305 or 4309, at least 85% identical to of any one of SEQ ID NOs: 743, 744, 1036, 1056, 1178, 1521, 2148, 2151, 2152, 2158, 2619, 2882, 2883, 2884, 3042, 3305, 3969, 4216, 4305 or 4309, at least 90% identical to any one of SEQ ID NOs: 743, 744, 1036, 1056, 1178, 1521, 2148, 2151, 2152, 2158, 2619, 2882, 2883, 2884, 3042, 3305, 3969, 4216, 4305 or 4309, or at least 95% identical to any one of SEQ ID NOs: 743, 744, 1036, 1056, 1178, 1521, 2148, 2151, 2152, 2158, 2619, 2882, 2883, 2884, 3042, 3305, 3969, 4216, 4305 or 4309. In some embodiments, the sense strand sequence comprises or consists of the sequence of any one of SEQ ID NOs: 743, 744, 1036, 1056, 1178, 1521, 2148, 2151, 2152, 2158, 2619, 2882, 2883, 2884, 3042, 3305, 3969, 4216, 4305 or 4309, or a sense strand sequence thereof having 1, 2, 3, or 4 nucleoside substitutions, additions, or deletions. In some embodiments, the sense strand sequence comprises or consists of the sequence of any one of SEQ ID NOs: 743, 744, 1036, 1056, 1178, 1521, 2148, 2151, 2152, 2158, 2619, 2882, 2883, 2884, 3042, 3305, 3969, 4216, 4305 or 4309, or a sense strand sequence thereof having 1 or 2 nucleoside substitutions, additions, or deletions. In some embodiments, the sense strand sequence comprises or consists of a sequence 100% identical to SEQ ID NOs: 743, 744, 1036, 1056, 1178, 1521, 2148, 2151, 2152, 2158, 2619, 2882, 2883, 2884, 3042, 3305, 3969, 4216, 4305 or 4309. The sense strand may comprise a modification pattern described herein. The sense strand may comprise a lipid moiety. The sense strand may comprise a GalNAc moiety.

In some embodiments, the siRNA comprises an antisense strand having a sequence in accordance with any of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799. In some embodiments, the antisense strand sequence comprises or consists of sequence at least 75% identical to any one of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799, at least 80% identical to any one of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799, at least 85% identical to of any one of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799, at least 90% identical to any one of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799, or at least 95% identical to any one of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799. In some embodiments, the antisense strand sequence comprises or consists of the sequence of any one of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799, or an antisense strand sequence thereof having 1, 2, 3, or 4 nucleoside substitutions, additions, or deletions. In some embodiments, the antisense strand sequence comprises or consists of the sequence of any one of SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799, or an antisense strand sequence thereof having 1 or 2 nucleoside substitutions, additions, or deletions. In some embodiments, the antisense strand sequence comprises or consists of a sequence 100% identical to SEQ ID NOs: 6233, 6234, 6526, 6546, 6668, 7011, 7638, 7641, 7642, 7648, 8109, 8372, 8373, 8374, 8532, 8795, 9459, 9706, 9795, or 9799. The antisense strand may comprise a modification pattern described herein. The antisense strand may comprise a lipid moiety. The antisense strand may comprise a GalNAc moiety.

In some embodiments, the siRNA comprises a sense strand having a sequence in accordance with any of SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756. In some embodiments, the sense strand sequence comprises or consists of sequence at least 75% identical to any one of SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756, at least 80% identical to any one of SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756, at least 85% identical to of any one of SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756, at least 90% identical to any one of SEQ ID NOs: 1-5490, or at least 95% identical to any one of SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756. In some embodiments, the sense strand sequence comprises or consists of the sequence of any one of SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756, or a sense strand sequence thereof having 1, 2, 3, or 4 nucleoside substitutions, additions, or deletions. In some embodiments, the sense strand sequence comprises or consists of the sequence of any one of SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756, or a sense strand sequence thereof having 1 or 2 nucleoside substitutions, additions, or deletions. In some embodiments, the sense strand sequence comprises or consists of a sequence 100% identical to SEQ ID NOs: 888, 1683, 1684, 1912, 1922, 1927, 1938, 2014, 2167, 2213, 2362, 2364, 2365, 2598, 2631, 2759, 2890, 3231, 3259, 3333, 3487, 3491, 3586, 3587, or 3756. The sense strand may comprise a modification pattern described herein. The sense strand may comprise a lipid moiety. The sense strand may comprise a GalNAc moiety.

In some embodiments, the siRNA comprises an antisense strand having a sequence in accordance with any of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246. In some embodiments, the antisense strand sequence comprises or consists of sequence at least 75% identical to any one of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246, at least 80% identical to any one of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246, at least 85% identical to of any one of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246, at least 90% identical to any one of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246, or at least 95% identical to any one of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246. In some embodiments, the antisense strand sequence comprises or consists of the sequence of any one of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246, or an antisense strand sequence thereof having 1, 2, 3, or 4 nucleoside substitutions, additions, or deletions. In some embodiments, the antisense strand sequence comprises or consists of the sequence of any one of SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246, or an antisense strand sequence thereof having 1 or 2 nucleoside substitutions, additions, or deletions. In some embodiments, the antisense strand sequence comprises or consists of a sequence 100% identical to SEQ ID NOs: 6378, 7173, 7174, 7402, 7412, 7417, 7428, 7504, 7657, 7703, 7852, 7854, 7855, 8088, 8121, 8249, 8380, 8721, 8749, 8823, 8977, 8981, 9076, 9077, or 9246. The antisense strand may comprise a modification pattern described herein. The antisense strand may comprise a lipid moiety. The antisense strand may comprise a GalNAc moiety.

### B. ASOs

In some embodiments, the composition comprises an oligonucleotide that inhibits the expression of SOS2, wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length.

In some embodiments, the composition comprises an oligonucleotide that inhibits the expression of SOS2, wherein the oligonucleotide comprises an ASO about 12-30 nucleosides in length and comprising a nucleoside sequence complementary to about 12-30 contiguous nucleosides of a full-length human SOS2 mRNA sequence such as SEQ ID NO: 11253; wherein (i) the oligonucleotide comprises a modification comprising a modified nucleoside and/or a modified internucleoside linkage, and/or (ii) the composition comprises a pharmaceutically acceptable carrier. In some embodiments, the ASO comprise a nucleoside sequence complementary to at least about 10, 11, 12, 13, 14, 15, 16, 17, 18, 19, 20, 21, 22, 23, 24, 25, 26, 27, 28, 29, 30, or more contiguous nucleosides of one of SEQ ID NO: 11253.

### C. Modifications

In some embodiments, the composition comprises an oligonucleotide that inhibits the expression of SOS2, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. Benefits of the modified internucleoside linkage may include decreased toxicity or improved pharmacokinetics.

In some embodiments, the composition comprises an oligonucleotide that inhibits the expression of SOS2, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that inhibits the expression of SOS2, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

In some embodiments, the composition comprises an oligonucleotide that inhibits the expression of SOS2, wherein the oligonucleotide comprises an arginine-glycine-aspartic acid (RGD) peptide. In some embodiments, the composition comprises an RGD peptide. In some embodiments, the composition comprises an RGD peptide derivative. In some embodiments, the RGD peptide is attached at a 3′ terminus of the oligonucleotide. In some embodiments, the RGD peptide is attached at a 5′ terminus of the oligonucleotide. In some embodiments, the composition comprises a sense strand, and the RGD peptide is attached to the sense strand (e.g. attached to a 5′ end of the sense strand, or attached to a 3′ end of the sense strand). In some embodiments, the composition comprises an antisense strand, and the RGD peptide is attached to the antisense strand (e.g. attached to a 5′ end of the antisense strand, or attached to a 3′ end of the antisense strand). In some embodiments, the composition comprises an RGD peptide attached at a 3′ or 5′ terminus of the oligonucleotide. In some embodiments, the oligonucleotide comprises an RGD peptide and a lipid attached at a 3′ or 5′ terminus of the oligonucleotide. In some embodiments, the RGD peptide comprises Cyclo(-Arg-Gly-Asp-D-Phe-Cys). In some embodiments, the RGD peptide comprises Cyclo(-Arg-Gly-Asp-D-Phe-Lys). In some embodiments, the RGD peptide comprises Cyclo(-Arg-Gly-Asp-D-Phe-azido). In some embodiments, the RGD peptide comprises an amino benzoic acid derived RGD. In some embodiments, the RGD peptide comprises Cyclo(-Arg-Gly-Asp-D-Phe-Cys), Cyclo(-Arg-Gly-Asp-D-Phe-Lys), Cyclo(-Arg-Gly-Asp-D-Phe-azido), an amino benzoic acid derived RGD, or a combination thereof. In some embodiments, the RGD peptide comprises multiple of such RGD peptides. For example, the RGD peptide may include 2, 3, or 4 RGD peptides.

In some embodiments, the moiety includes a negatively charged group attached at a 5′ end of the oligonucleotide. This may be referred to as a 5′-end group. In some embodiments, the negatively charged group is attached at a 5′ end of an antisense strand of an siRNA disclosed herein. The 5′-end group may be or include a 5′-end phosphorothioate, 5′-end phosphorodithioate, 5′-end vinylphosphonate (5′-VP), 5′-end methylphosphonate, 5′-end cyclopropyl phosphonate, or a 5′-deoxy-5′-C-malonyl. The 5′-end group may comprise 5′-VP. In some embodiments, the 5′-VP comprises a trans-vinylphosphate or cis-vinylphosphate. The 5′-end group may include an extra 5′ phosphate. A combination of 5′-end groups may be used.

In some embodiments, the oligonucleotide includes a negatively charged group. The negatively charged group may aid in cell or tissue penetration. The negatively charged group may be attached at a 5′ or 3′ end (e.g. a 5′ end) of the oligonucleotide. This may be referred to as an end group. The end group may be or include a phosphorothioate, phosphorodithioate, vinylphosphonate, methylphosphonate, cyclopropyl phosphonate, or a deoxy-C-malonyl. The end group may include an extra 5′ phosphate such as an extra 5′ phosphate. A combination of end groups may be used.

In some embodiments, the oligonucleotide includes a phosphate mimic. In some embodiments, the phosphate mimic comprises vinyl phosphonate. In some embodiments, the vinyl phosphonate comprises a trans-vinylphosphate. In some embodiments, the vinyl phosphonate comprises a cis-vinylphosphate. An example of a nucleotide that includes a vinyl phosphonate is shown below.

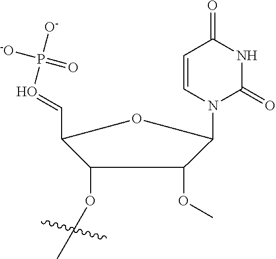

5′ vinylphosphonate 2′ O Methyl Uridine

In some embodiments, the vinyl phosphonate increases the stability of the oligonucleotide. In some embodiments, the vinyl phosphonate increases the accumulation of the oligonucleotide in tissues. In some embodiments, the vinyl phosphonate protects the oligonucleotide from an exonuclease or a phosphatase. In some embodiments, the vinyl phosphonate improves the binding affinity of the oligonucleotide with the siRNA processing machinery.

In some embodiments, the oligonucleotide includes 1 vinyl phosphonate. In some embodiments, the oligonucleotide includes 2 vinyl phosphonates. In some embodiments, the oligonucleotide includes 3 vinyl phosphonates. In some embodiments, the oligonucleotide includes 4 vinyl phosphonates. In some embodiments, the antisense strand of the oligonucleotide comprises a vinyl phosphonate at the 5′ end. In some embodiments, the antisense strand of the oligonucleotide comprises a vinyl phosphonate at the 3′ end. In some embodiments, the sense strand of the oligonucleotide comprises a vinyl phosphonate at the 5′ end. In some embodiments, the sense strand of the oligonucleotide comprises a vinyl phosphonate at the 3′ end.

### D. Hydrophobic Moieties

In some embodiments, the composition comprises an oligonucleotide that inhibits the expression of SOS2, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

In some embodiments, the composition comprises an oligonucleotide that inhibits the expression of SOS2, wherein the oligonucleotide comprises a hydrophobic ligand or moiety. In some embodiments, the hydrophobic ligand or moiety comprises cholesterol. In some embodiments, the hydrophobic ligand or moiety comprises a cholesterol derivative. In some embodiments, the hydrophobic ligand or moiety is attached at a 3′ terminus of the oligonucleotide. In some embodiments, the hydrophobic ligand or moiety s attached at a 5′ terminus of the oligonucleotide. In some embodiments, the composition comprises a sense strand, and the hydrophobic ligand or moiety is attached to the sense strand (e.g. attached to a 5′ end of the sense strand, or attached to a 3′ end of the sense strand). In some embodiments, the composition comprises an antisense strand, and the hydrophobic ligand or moiety is attached to the antisense strand (e.g. attached to a 5′ end of the antisense strand, or attached to a 3′ end of the antisense strand). In some embodiments, the composition comprises a hydrophobic ligand or moiety attached at a 3′ or 5′ terminus of the oligonucleotide.

In some embodiments, a hydrophobic moiety is attached to the oligonucleotide (e.g. a sense strand and/or an antisense strand of a siRNA). In some embodiments, a hydrophobic moiety is attached at a 3′ terminus of the oligonucleotide. In some embodiments, a hydrophobic moiety is attached at a 5′ terminus of the oligonucleotide. In some embodiments, the hydrophobic moiety comprises cholesterol. In some embodiments, the hydrophobic moiety includes a cyclohexanyl.

In some embodiments, the composition comprises an oligonucleotide that inhibits the expression of SOS, wherein the oligonucleotide comprises a lipid attached at a 3′ or 5′ terminus of the oligonucleotide. In some embodiments, a lipid is attached at a 3′ terminus of the oligonucleotide. In some embodiments, a lipid is attached at a 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the lipid comprises stearyl, lithocholyl, docosanyl, docosahexaenyl, or myristyl. In some embodiments, the lipid comprises cholesterol. In some embodiments, the lipid includes a sterol such as cholesterol. In some embodiments, the lipid comprises stearyl, t-butylphenol, n-butylphenol, octylphenol, dodecylphenol, phenyl n-dodecyl, octadecylbenzamide, hexadecylbenzamide, or octadecylcyclohexyl. In some embodiments, the lipid comprises phenyl para C12.

In some embodiments, the oligonucleotide comprises any aspect of the following structure:

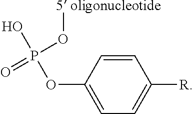

In some embodiments, the oligonucleotide comprises any aspect of the following structure:

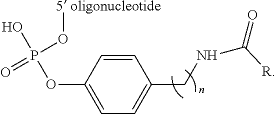

In some embodiments, the oligonucleotide comprises any aspect of the following structure:

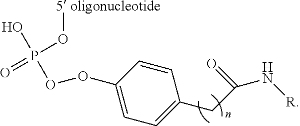

In some embodiments, the oligonucleotide comprises any aspect of the following structure: The aspect included in the oligonucleotide may include the entire structure, or may include the lipid moiety, of any of the structures shown. In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, the alkyl group contains 4-18 carbons. In some embodiments, the lipid moiety comprises an alcohol or ether.

In some embodiments, the lipid includes a fatty acid. In some embodiments, the lipid comprises a lipid depicted in Table 1. The example lipid moieties in Table 1 are shown attached at a 5′ end of an oligonucleotide, in which the 5′ terminal phosphate of the oligonucleotide is shown with the lipid moiety. In some embodiments, a lipid moiety in Table 1 may be attached at a different point of attachment than shown. For example, the point of attachment of any of the lipid moieties in the table may be at a 3′ oligonucleotide end. In some embodiments, the lipid is used for targeting the oligonucleotide to a non-hepatic cell or tissue.

TABLE 1
Hydrophobic moiety examples

| Hydrophobic | Hydrophobic | |
|---|---|---|
| Moiety Description | Moiety Name | Example Conjugation |
| stearyl | ETL3 | 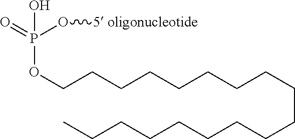 |
| t-butylphenyl | ETL7 | 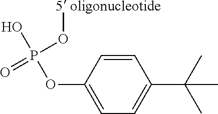 |
| n-butylphenyl | ETL8 | 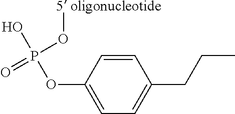 |
| octylphenyl | ETL9 | 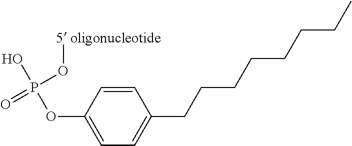 |
| dodecylphenyl | ETL10 | 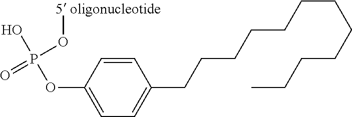 |
| phenyl n-dodecyl | ETL12 | 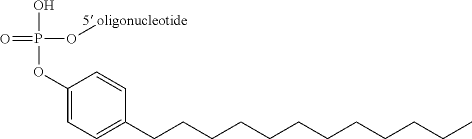 |
| octadecylbenzamide | ETL13 | 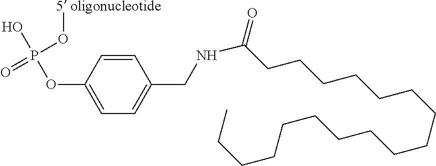 |
| hexadecylbenzamide | ETL15 | 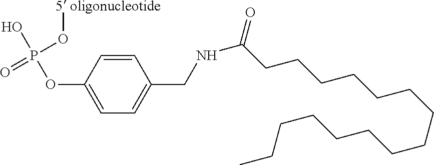 |
| octadecylcyclohexyl | ETL16 | 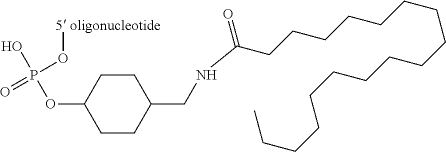 |

In some embodiments, the lipid or lipid moiety includes 16 to 18 carbons. In some embodiments, the lipid includes 16 carbons. In some embodiments, the lipid includes 17 carbons. In some embodiments, the lipid includes 18 carbons. In some embodiments, the lipid moiety includes 16 carbons. In some embodiments, the lipid moiety includes 17 carbons. In some embodiments, the lipid moiety includes 18 carbons.

The hydrophobic moiety may include a linker that comprises a carbocycle. The carbocycle may be six-membered. Some examples of a carbocycle include phenyl or cyclohexyl. The linker may include a phenyl. The linker may include a cyclohexyl. The lipid may be attached to the carbocycle, which may in turn be attached at a phosphate (e.g. 5′ or 3′ phosphate) of the oligonucleotide. In some embodiments, the lipid or hydrocarbon, and the end of the sense are connected to the phenyl or cyclohexyl linker in the 1,4; 1,3; or 1,2 substitution pattern (e.g. the para, meta, or ortho phenyl configuration). In some embodiments, the lipid or hydrocarbon, and the end of the sense are connected to the phenyl or cyclohexyl linker in the 1,4 substitution pattern (e.g. the para phenyl configuration). The lipid may be attached to the carbocycle in the 1,4 substitution pattern relative to the oligonucleotide. The lipid may be attached to the carbocycle in the 1,3 substitution pattern relative to the oligonucleotide. The lipid may be attached to the carbocycle in the 1,2 substitution pattern relative to the oligonucleotide. The lipid may be attached to the carbocycle in the ortho orientation relative to the oligonucleotide. The lipid may be attached to the carbocycle in the para orientation relative to the oligonucleotide. The lipid may be attached to the carbocycle in the meta orientation relative to the oligonucleotide.

The lipid moiety may comprise or consist of the following structure

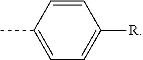

In some embodiments, the lipid moiety comprises or consists of the following structure:

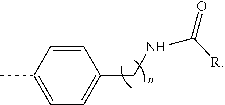

In some embodiments, the lipid moiety comprises the following structure:

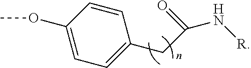

In some embodiments, the lipid moiety comprises or consist of the following structure:

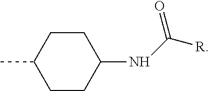

In some embodiments, the dotted line indicates a covalent connection. The covalent connection may between an end of the sense or antisense strand. For example, the connection may be to the 5′ end of the sense strand. In some embodiments, n is 0-3. In some embodiments, n is 1-3. In some embodiments, n is 0. In some embodiments, n is 1. In some embodiments, n is 2. In some embodiments, n is 3. In some embodiments, n is 4. In some embodiments, n is 5. In some embodiments, n is 1, 2, 3, 4, 5, 6, 7, 8, 9, or 10. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons.

The lipid moiety may be attached at a 5′ end of the oligonucleotide. The 5′ end may have one phosphate linking the lipid moiety to a 5′ carbon of a sugar of the oligonucleotide. The 5′ end may have two phosphates linking the lipid moiety to a 5′ carbon of a sugar of the oligonucleotide. The 5′ end may have three phosphates linking the lipid moiety to a 5′ carbon of a sugar of the oligonucleotide. The 5′ end may have one phosphate connected to the 5′ carbon of a sugar of the oligonucleotide, where the one phosphate is connected to the lipid moiety. The 5′ end may have two phosphates connected to the 5′ carbon of a sugar of the oligonucleotide, where the one of the two phosphates is connected to the lipid moiety. The 5′ end may have three phosphates connected to the 5′ carbon of a sugar of the oligonucleotide, where the one of the three phosphates is connected to the lipid moiety. The sugar may include a ribose. The sugar may include a deoxyribose. The sugar may be modified a such as a 2′ modified sugar (e.g. a 2′ O-methyl or 2′ fluoro ribose). A phosphate of the 5′ end may include a modification such as a sulfur in place of an oxygen. Two phosphates of the 5′ end may include a modification such as a sulfur in place of an oxygen. Three phosphates of the 5′ end may include a modification such as a sulfur in place of an oxygen.

In some embodiments, the oligonucleotide includes 1 lipid moiety. In some embodiments, the oligonucleotide includes 2 lipid moieties. In some embodiments, the oligonucleotide includes 3 lipid moieties. In some embodiments, the oligonucleotide includes 4 lipid moieties.

Some embodiments relate to a method of making an oligonucleotide comprising a hydrophobic conjugate. A strategy for making hydrophobic conjugates may include use of a phosphoramidite reagent based upon a 6-membered ring alcohol such as a phenol or cyclohexanol. The phosphoramidite may be reacted to a nucleotide to connect the nucleotide to the hydrophobic moiety, and thereby produce the hydrophobic conjugate. Some examples of phosphoramidite reagents that may be used to produce a hydrophobic conjugate are provided as follows:

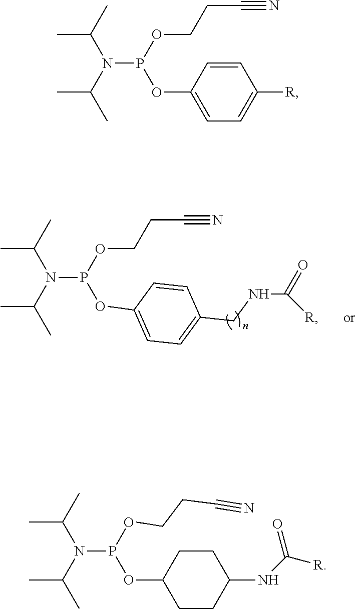

In some embodiments, n is 1-3. In some embodiments, n is 1. In some embodiments, n is 2. In some embodiments, n is 3. In some embodiments, R is an alkyl group. In some embodiments, the alkyl group contains 1, 2, 3, 4, 5, 6, 7, 8, 9, 10, 11, 12, 13, 14, 15, 16, 17, 18, 19, or 20 carbons. In some embodiments, the alkyl group contains 4, 5, 6, 7, 8, 9, 10, 11, 12, 13, 14, 15, 16, 17, or 18 carbons, or a range defined by any two of the aforementioned numbers of carbons. In some embodiments, R comprises or consists of an alkyl group containing 4-18 carbons. Any one of the phosphoramidite reagents may be reacted to a 5′ end of an oligonucleotide to produce an oligonucleotide comprising a hydrophobic moiety. In some embodiments, the phosphoramidite reagents is reacted to a 5′ end of a sense strand of an siRNA. The sense strand may then be hybridized to an antisense strand to form a duplex. The hybridization may be performed by incubating the sense and antisense strands in solution at a given temperature. The temperature may be gradually reduced. The temperature may comprise or include a temperature comprising an annealing temperature for the sense and antisense strands. The temperature may be below or include a temperature below the annealing temperature for the sense and antisense strands. The temperature may be below a melting temperature of the sense and antisense strands.

The lipid may be attached to the oligonucleotide by a linker. The linker may include a polyethyleneglycol (e.g. tetraethyleneglycol).

### E. Sugar Moieties

In some embodiments, the composition comprises an oligonucleotide that inhibits the expression of SOS2, wherein the oligonucleotide comprises a sugar moiety. The sugar moiety may include an N-acetyl galactose moiety (e.g. an N-acetylgalactosamine (GalNAc) moiety), an N-acetyl glucose moiety (e.g. an N-acetylglucosamine (GlcNAc) moiety), a fucose moiety, or a mannose moiety. The sugar moiety may include 1, 2, 3, or more sugar molecules. The sugar moiety may be attached at a 3′ or 5′ terminus of the oligonucleotide. The sugar moiety may include an N-acetyl galactose moiety. The sugar moiety may include an N-acetylgalactosamine (GalNAc) moiety. The sugar moiety may include an N-acetyl glucose moiety. The sugar moiety may include N-acetylglucosamine (GlcNAc) moiety. The sugar moiety may include a fucose moiety. The sugar moiety may include a mannose moiety. N-acetyl glucose, GlcNAc, fucose, or mannose may be useful for targeting macrophages when they target or bind a mannose receptor such as CD206. The sugar moiety may be useful for binding or targeting an asialoglycoprotein receptor such as an asialoglycoprotein receptor of a hepatocyte. The GalNAc moiety may bind to an asialoglycoprotein receptor. The GalNAc moiety may target a hepatocyte.

In some embodiments, the composition comprises an oligonucleotide that inhibits the expression of SOS2, wherein the oligonucleotide comprises an N-acetylgalactosamine (GalNAc) moiety. GalNAc may be useful for hepatocyte targeting. The GalNAc moiety may include a bivalent or trivalent branched linker. The oligo may be attached to 1, 2 or 3 GalNAcs through a bivalent or trivalent branched linker. The GalNAc moiety may include 1, 2, 3, or more GalNAc molecules. The GalNAc moiety may be attached at a 3′ or 5′ terminus of the oligonucleotide.

In some embodiments, the composition comprises an oligonucleotide that inhibits the expression of SOS2, wherein the oligonucleotide comprises an N-acetylgalactosamine (GalNAc) ligand for hepatocyte targeting. In some embodiments, the composition comprises GalNAc. In some embodiments, the composition comprises a GalNAc derivative. In some embodiments, the GalNAc ligand is attached at a 3′ terminus of the oligonucleotide. In some embodiments, the GalNAc ligand is attached at a 5′ terminus of the oligonucleotide. In some embodiments, the composition comprises a sense strand, and the GalNAc ligand is attached to the sense strand (e.g. attached to a 5′ end of the sense strand, or attached to a 3′ end of the sense strand). In some embodiments, the composition comprises an antisense strand, and the GalNAc ligand is attached to the antisense strand (e.g. attached to a 5′ end of the antisense strand, or attached to a 3′ end of the antisense strand). In some embodiments, the composition comprises a GalNAc ligand attached at a 3′ or 5′ terminus of the oligonucleotide.

In some embodiments, described herein is a compound (e.g. oligonucleotide) represented by Formula (I) or (II):

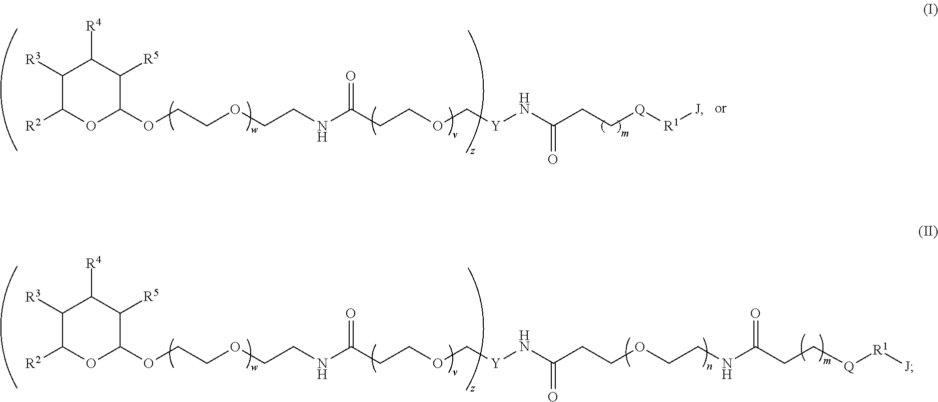

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
   if z is 3, Y is C
   if z is 2, Y is CR⁶, or
   if z is 1, Y is C(R⁶)₂;
Q is selected from:
   C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂;
R¹ is a linker selected from:
   —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from:
   C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
R³ and R⁴ are each independently selected from:
   —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from:
   —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
   hydrogen;
   halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
   C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁷ is independently selected from:
hydrogen;
   C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
   C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl.
      In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is selected from 1 and 2. In some embodiments, z is 3 and Y is C. In some embodiments, Q is selected from C₅₋₆ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, Q is selected from C₅₋₆ carbocycle optionally substituted with one or more substituents independently selected from halogen, —CN, —OH, —SH, —NO₂, and —NH₂. In some embodiments, Q is selected from phenyl and cyclohexyl, each of which is optionally substituted with one or more substituents independently selected from halogen, —CN, —OH, —SH, —NO₂, and —NH₂. In some embodiments, Q is selected from phenyl. In some embodiments, Q is selected from cyclohexyl. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂—NR⁷. In some embodiments, R¹ is selected from —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, and —OP(OR⁷)O—. In some embodiments, R¹ is selected from —OP(O)(OR⁷)O—, —OP(S)(OR⁷)O—, —OP(O)(O⁻)O—, —OP(S)(O⁻)O—, —OP(O)(S⁻)O—, and —OP(OR⁷)O—. In some embodiments, R¹ is selected from —OP(O)(OR⁷)O— and —OP(OR⁷)O—. In some embodiments, R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, R² is selected from C₁₋₃ alkyl substituted with one or more substituents independently selected from —OR⁷, —OC(O)R⁷, —SR⁷, and —N(R⁷)₂. In some embodiments, R² is selected from C₁₋₃ alkyl substituted with one or more substituents independently selected from —OR⁷ and —OC(O)R⁷. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from —OR⁷, —SR⁷, —OC(O)R⁷, and —N(R⁷)₂. In some embodiments, R³ is selected from —OR⁷— and —OC(O)R⁷. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from —OR⁷, —SR⁷, —OC(O)R⁷, and —N(R⁷)₂. In some embodiments, R⁴ is selected from —OR⁷— and —OC(O)R⁷. In some embodiments, R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, R⁵ is selected from —OC(O)R⁷ and —N(R⁷)C(O)R⁷. In some embodiments, each R⁷ is independently selected from: hydrogen; and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from C₁₋₆ alkyl optionally substituted with one or more substituents independently selected from halogen, —CN, —OH, and —SH. In some embodiments, w is 1; v is 1; n is 2; m is 1 or 2; z is 3 and Y is C; Q is phenyl or cyclohexyl, each of which is optionally substituted with one or more substituents independently selected from halogen, —CN, —OH, —SH, —NO₂, —NH₂, and C₁₋₃ alkyl; R¹ is selected from —OP(O)(OR⁷)O—, —OP(S)(OR⁷)O—, —OP(O)(O⁻)O—, —OP(S)(O⁻)O—, —OP(O)(S⁻)O— and —OP(OR⁷)O—; R² is C₁ alkyl substituted with —OH or —OC(O)CH₃;
R³ is —OH or —OC(O)CH₃; R⁴ is —OH or —OC(O)CH₃; and R⁵ is —NH(O)CH₃. In some embodiments, the compound comprises:

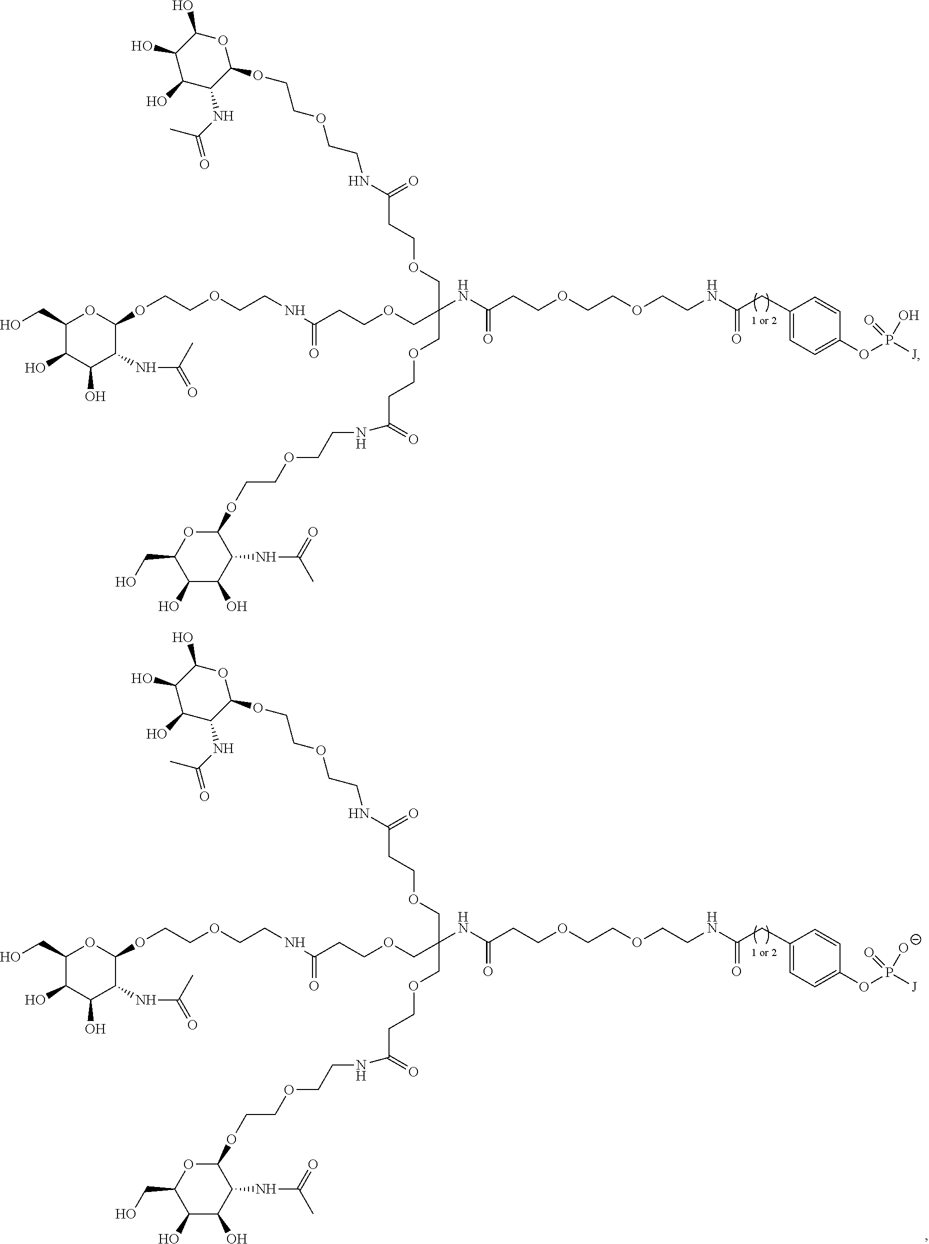

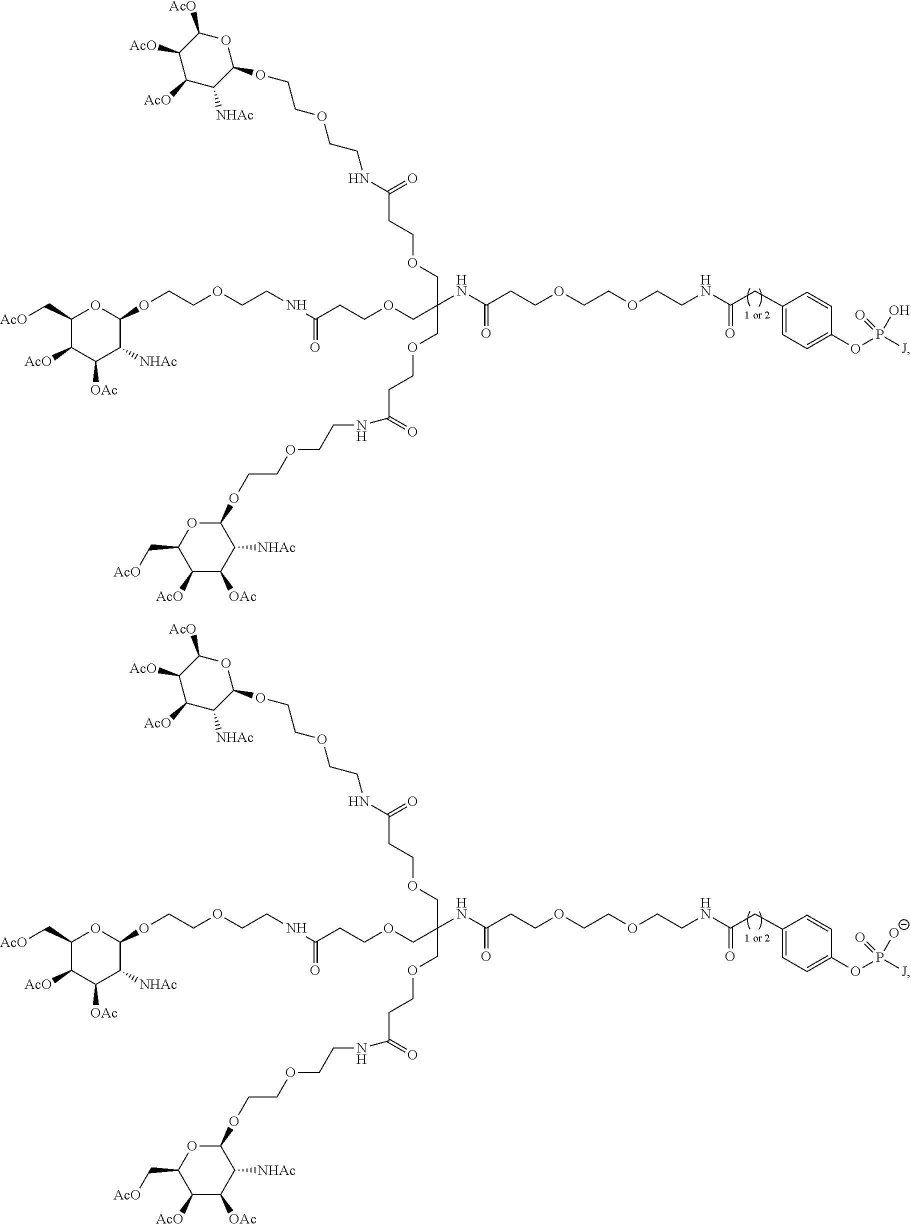

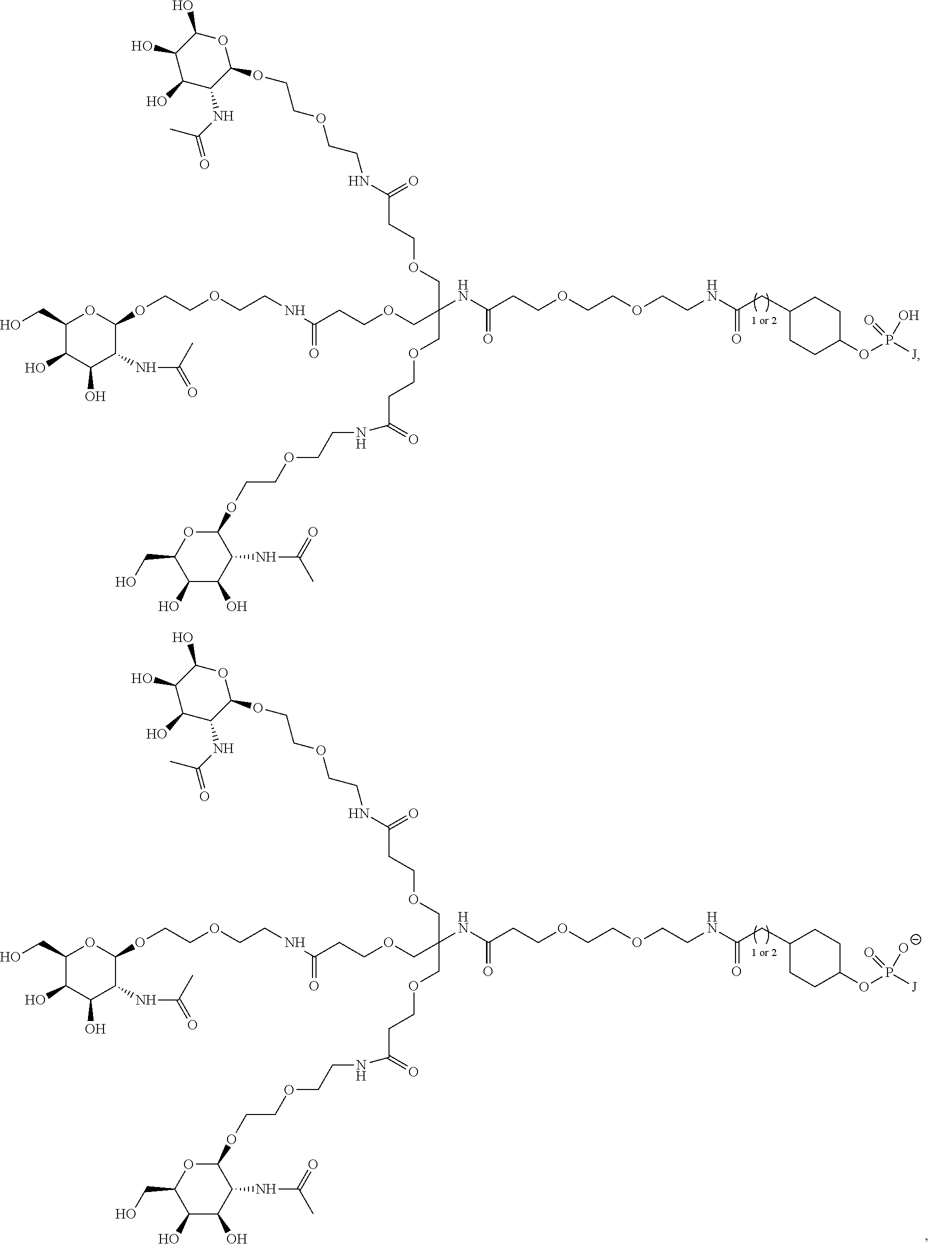

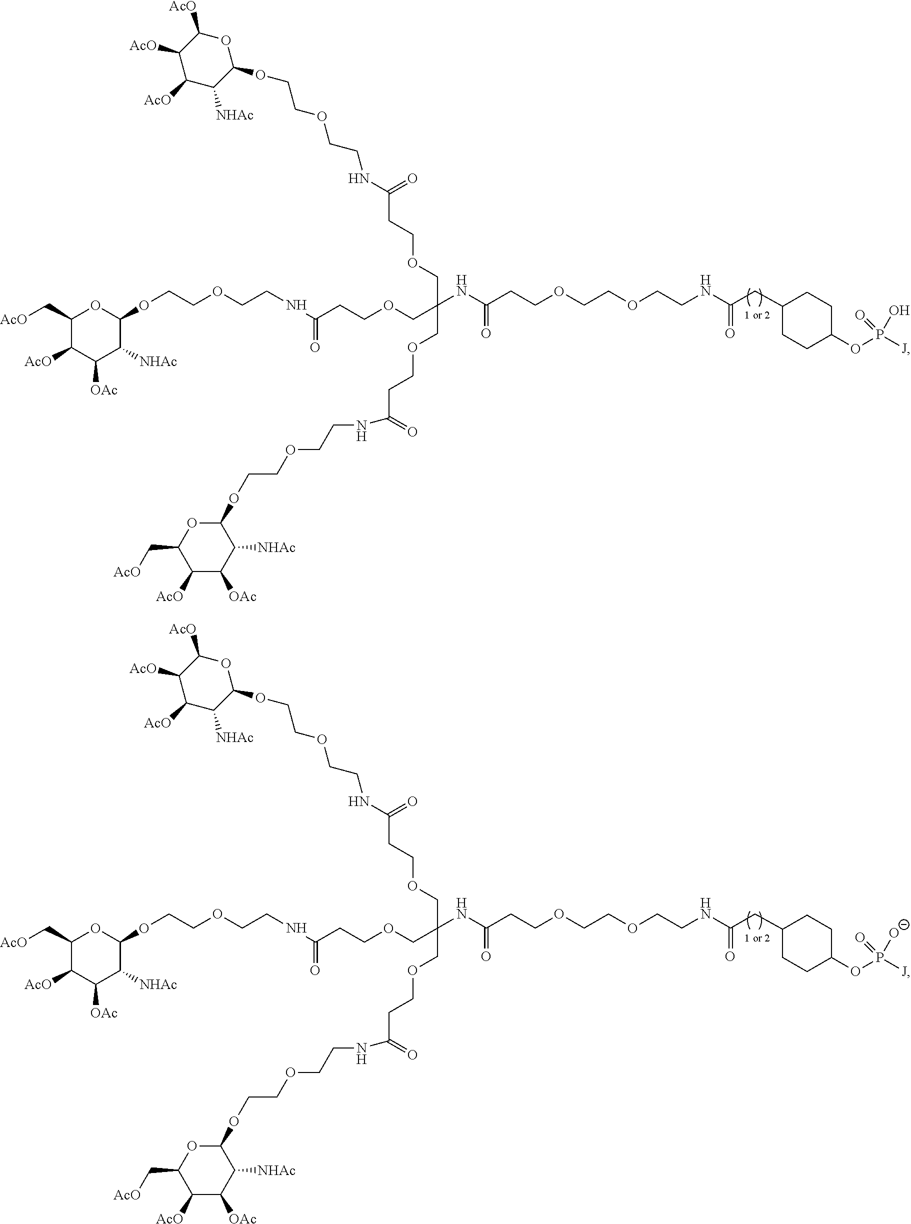

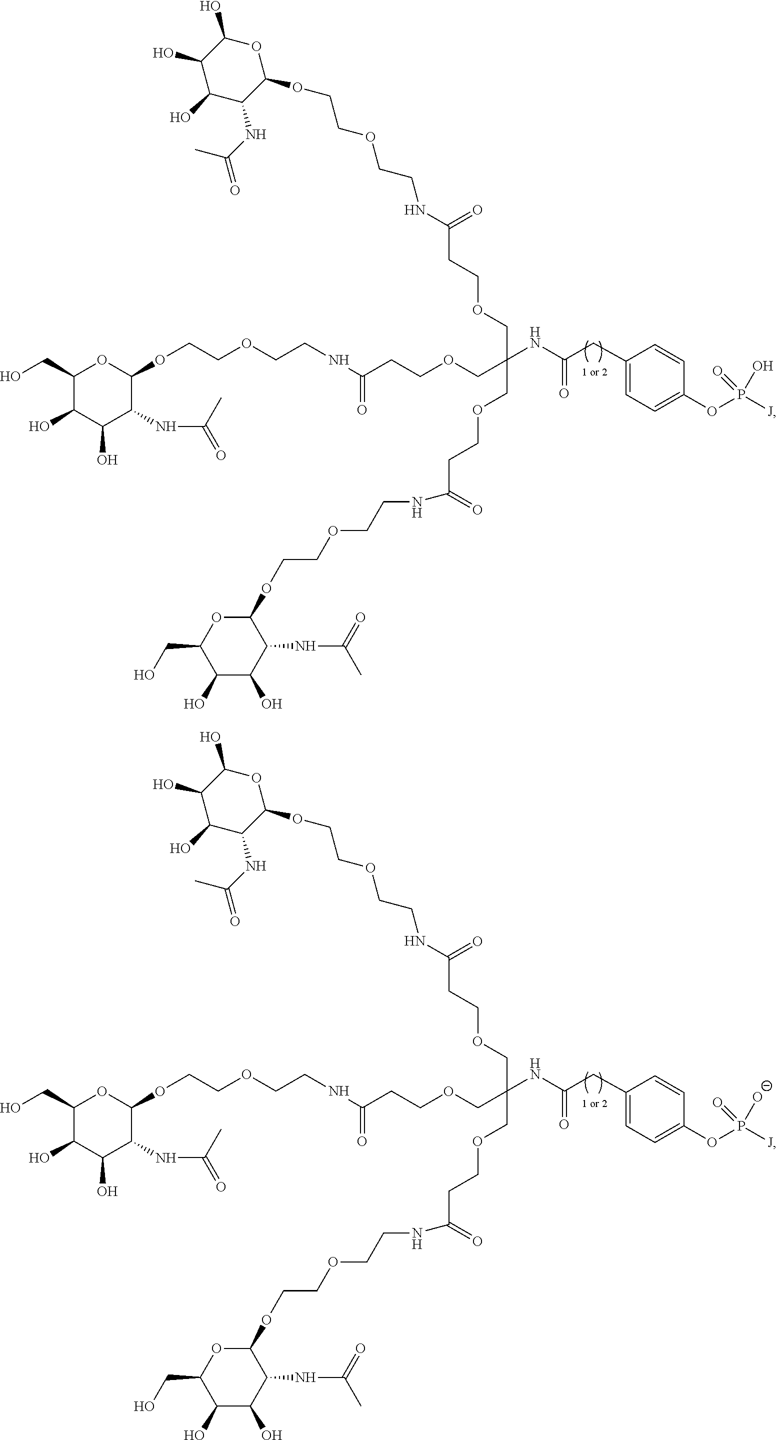

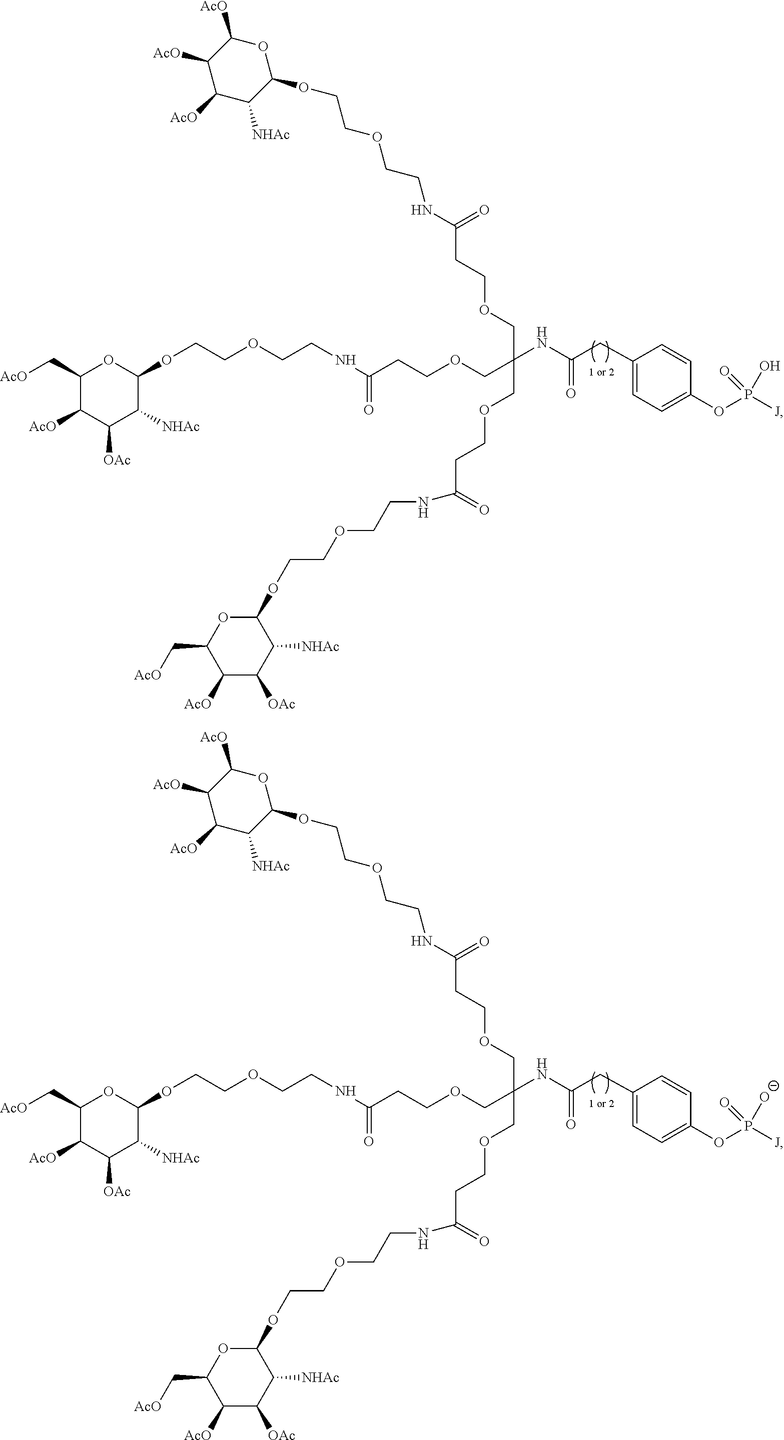

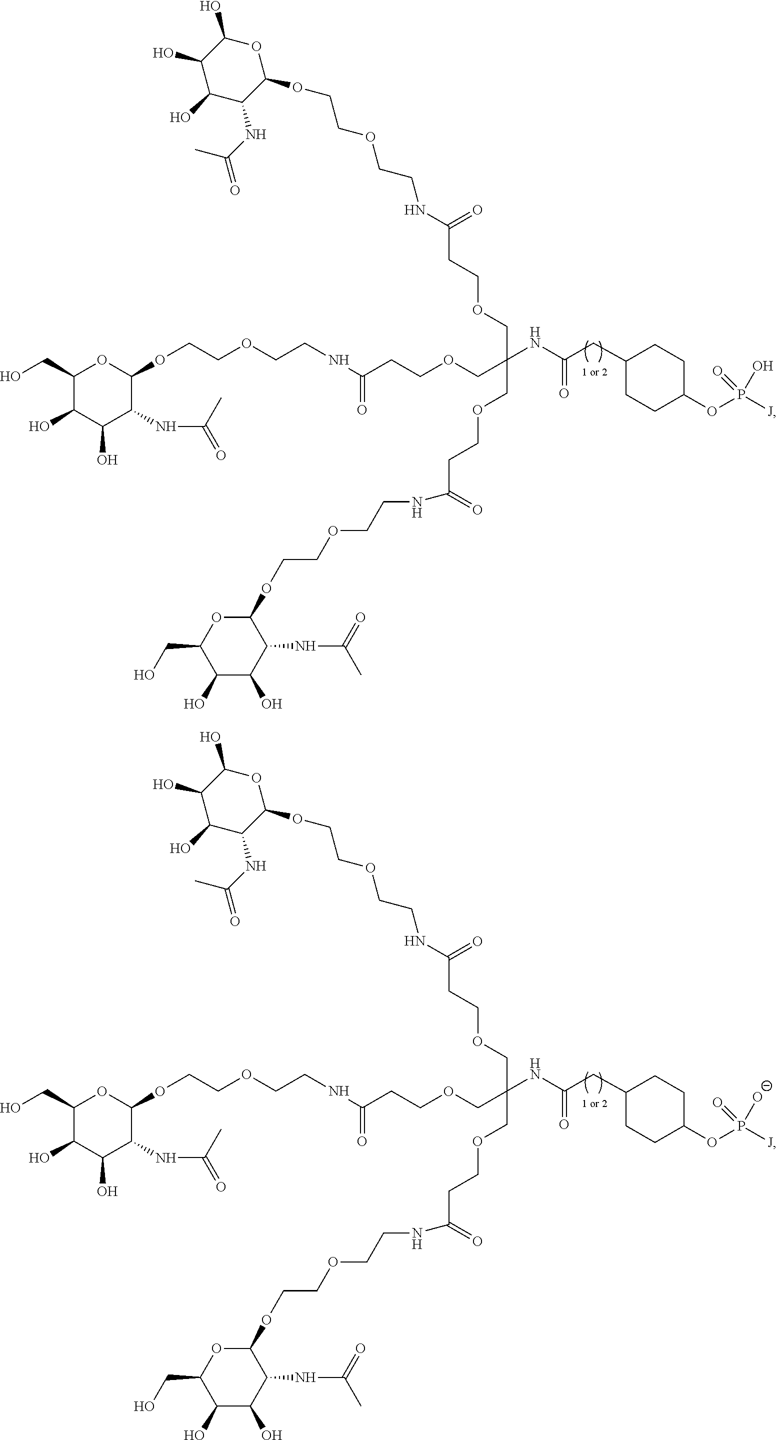

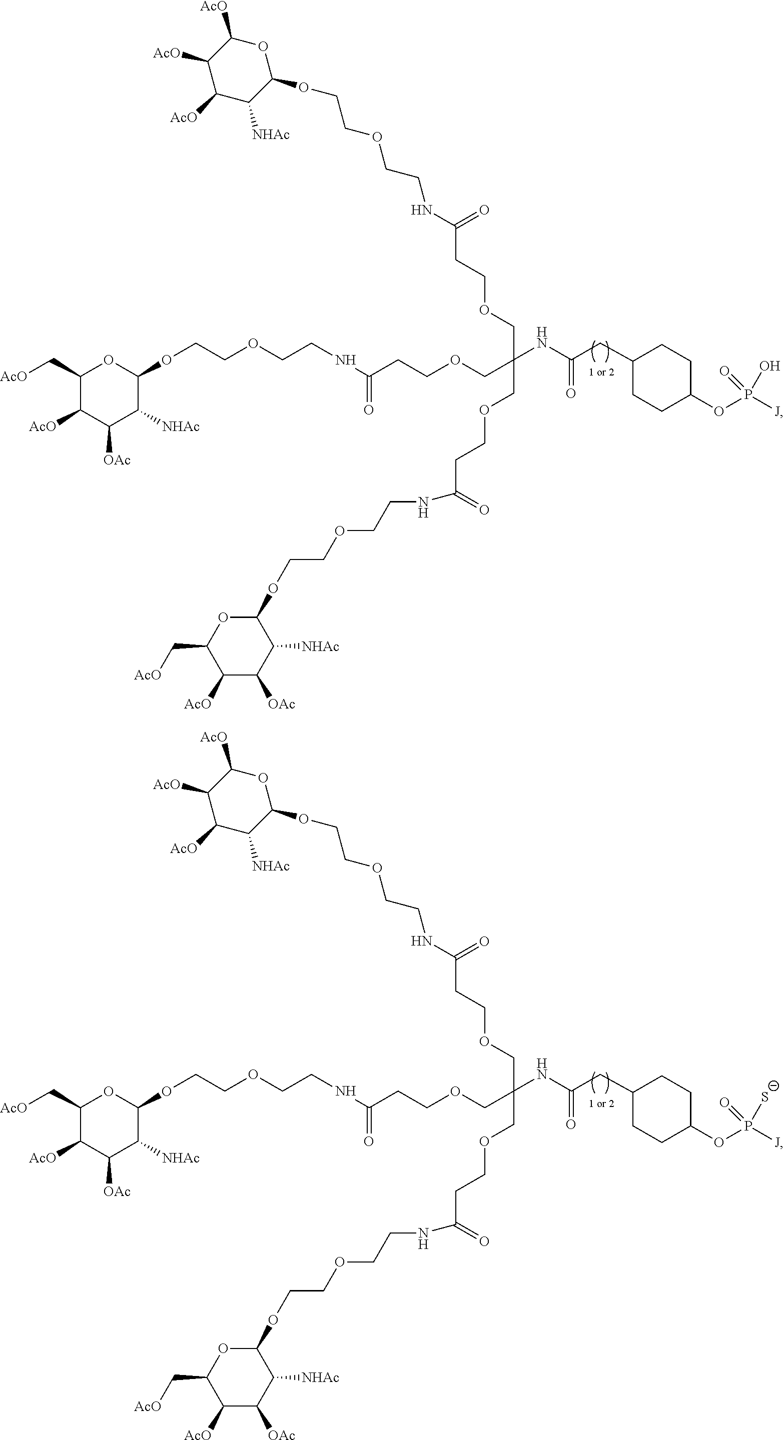

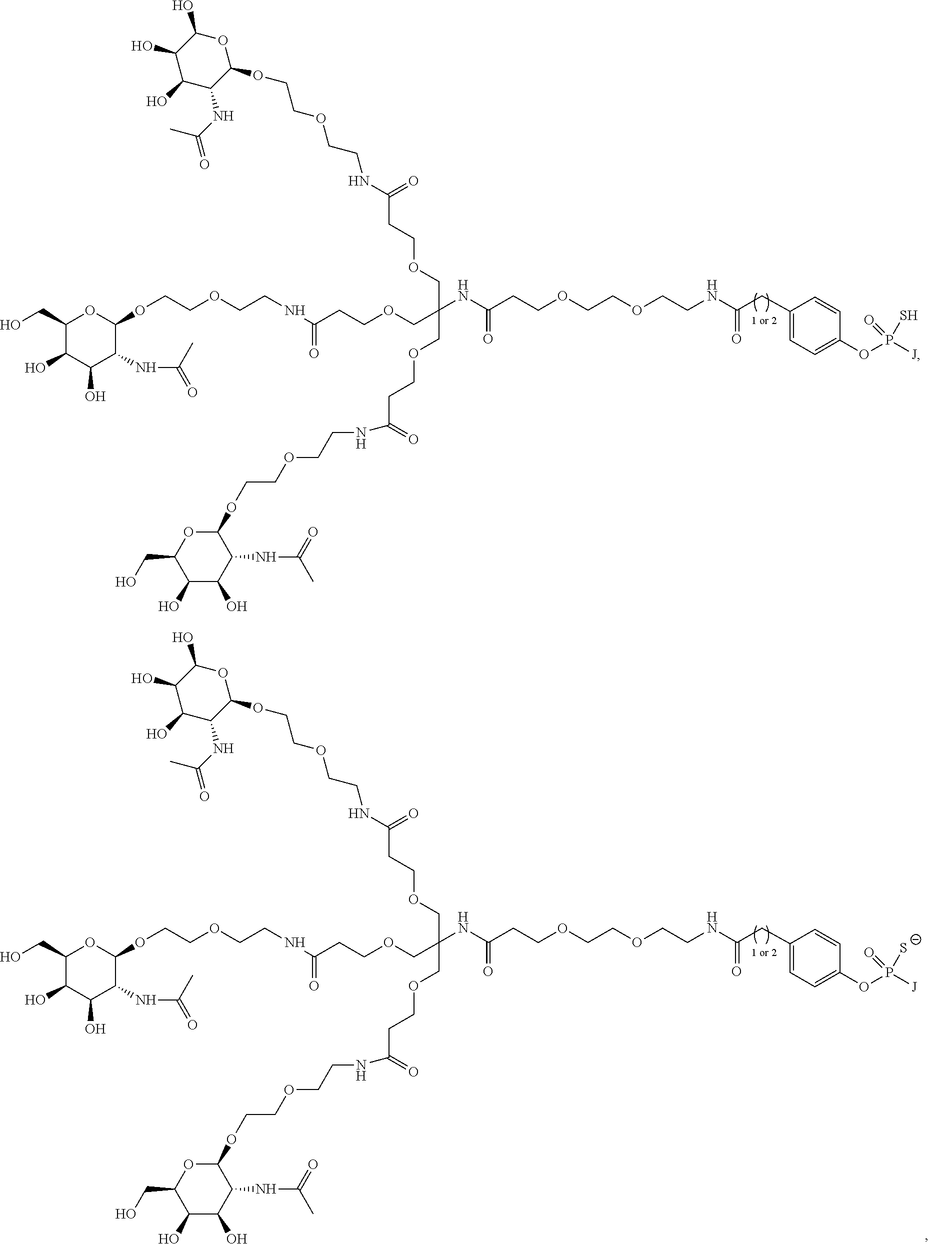

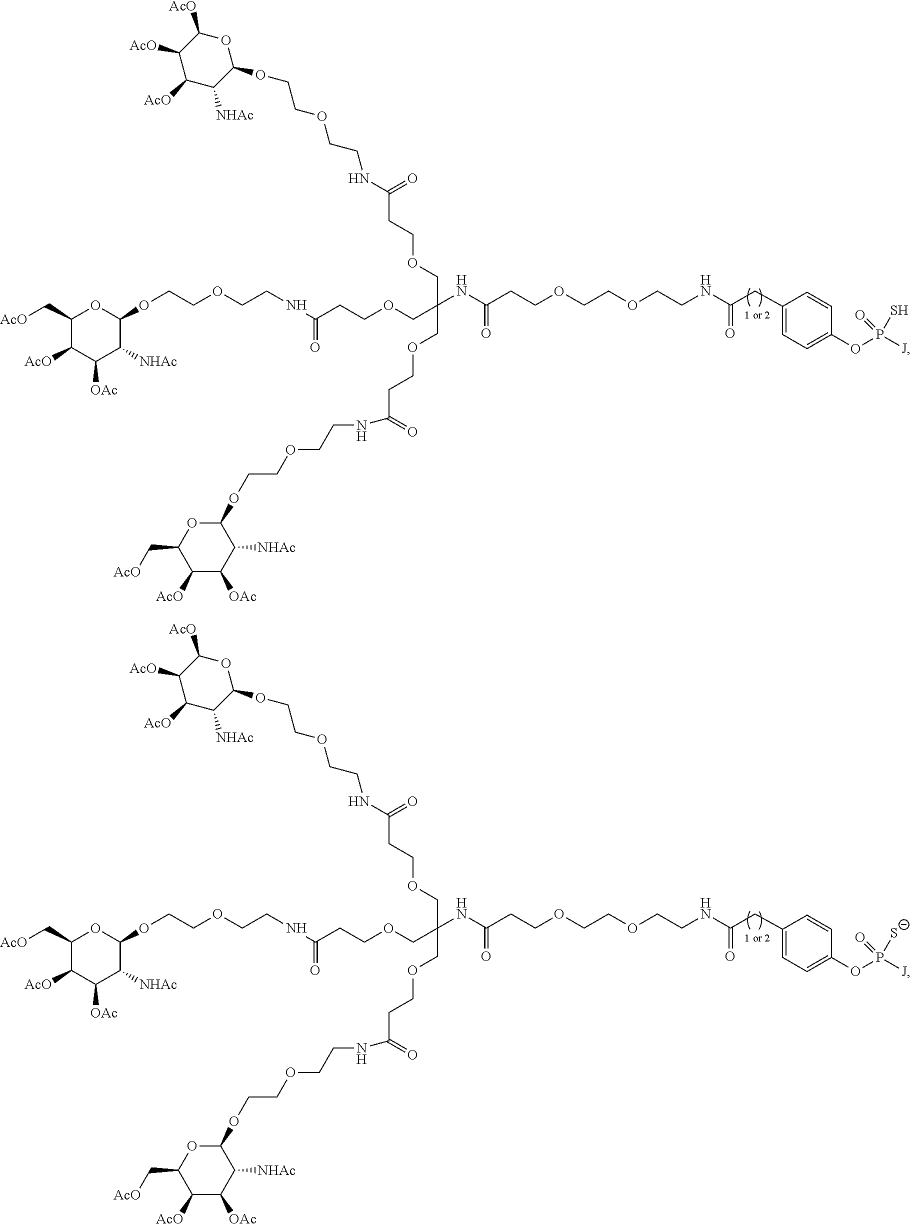

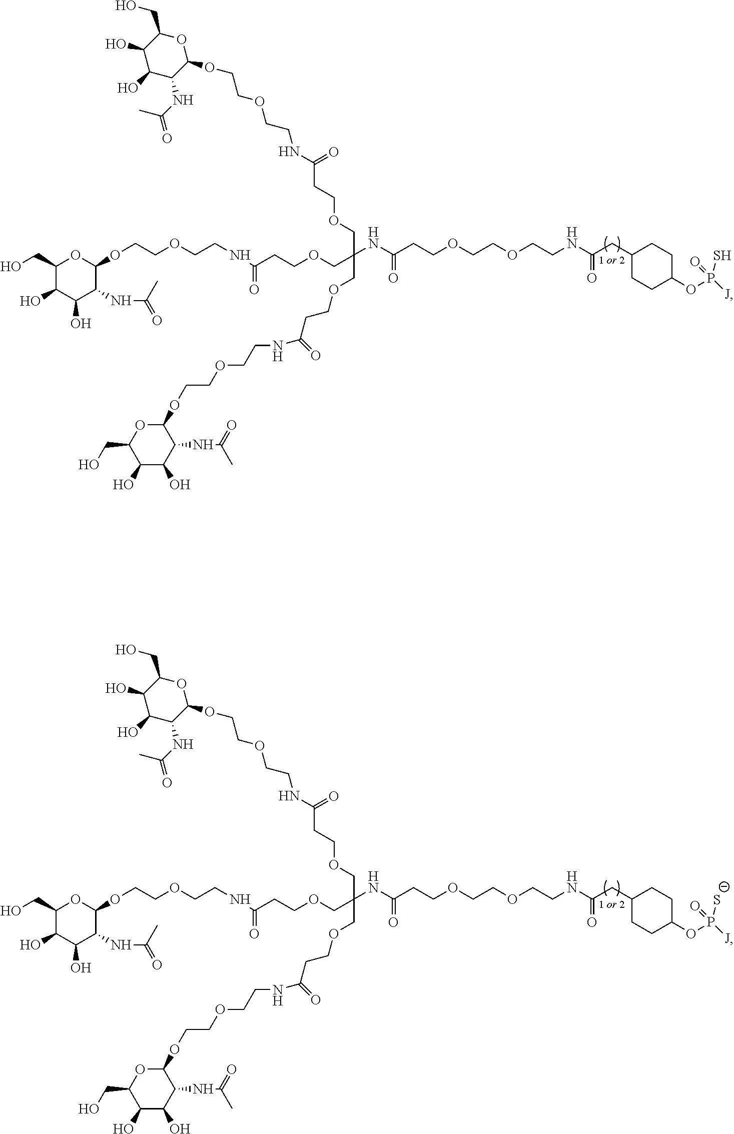

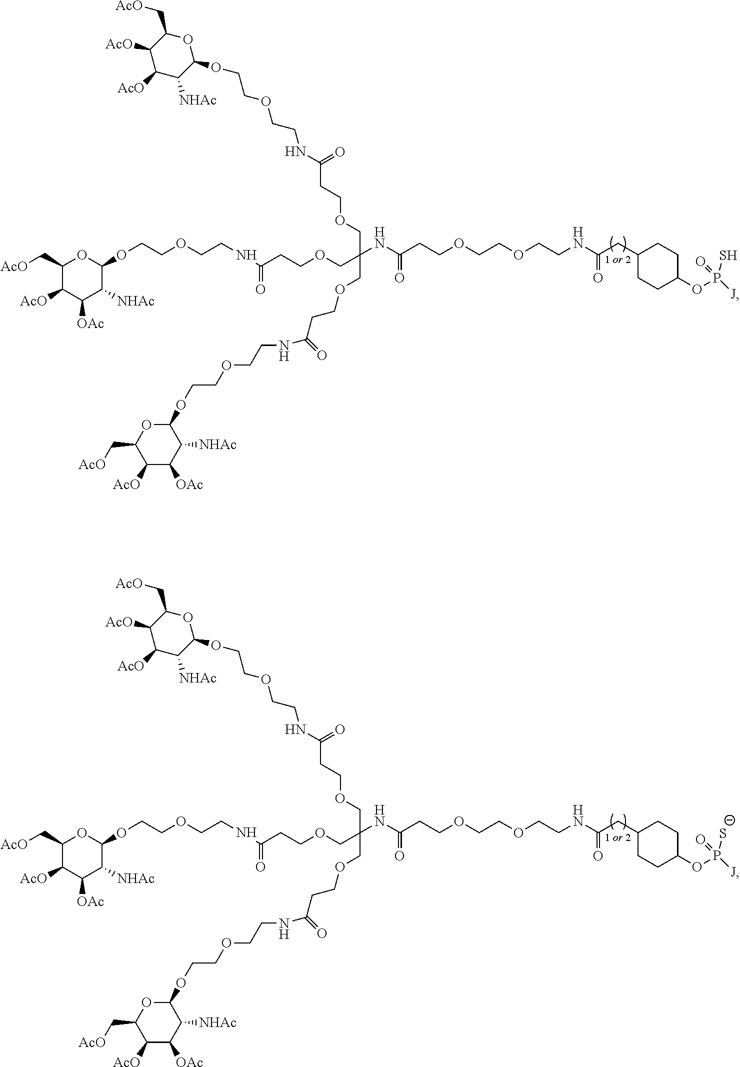

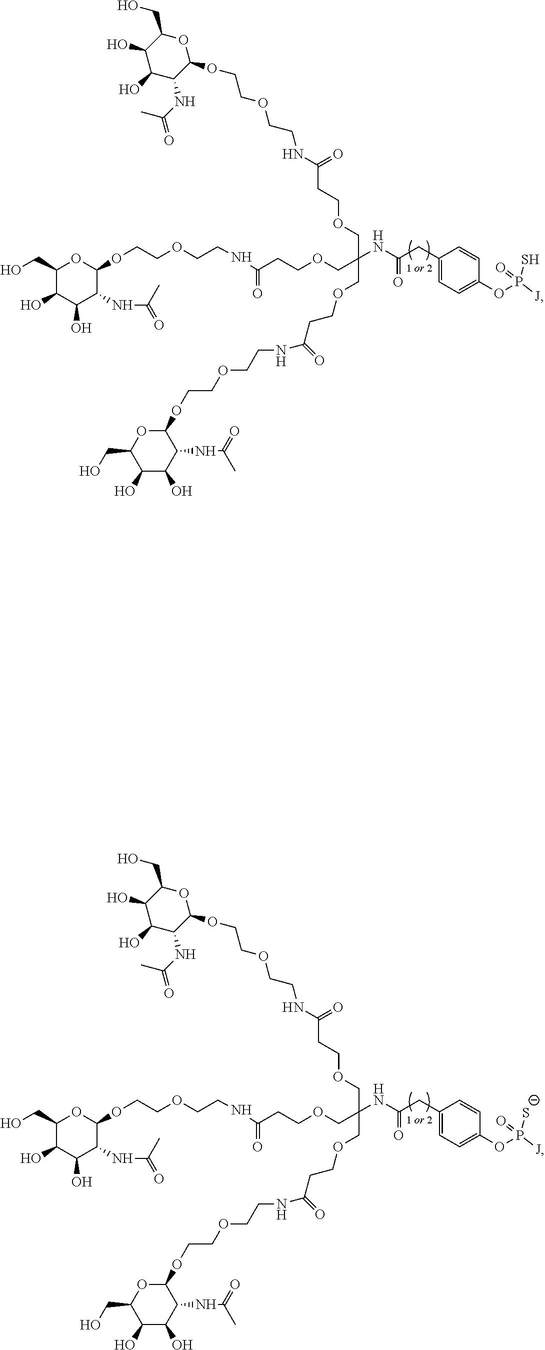

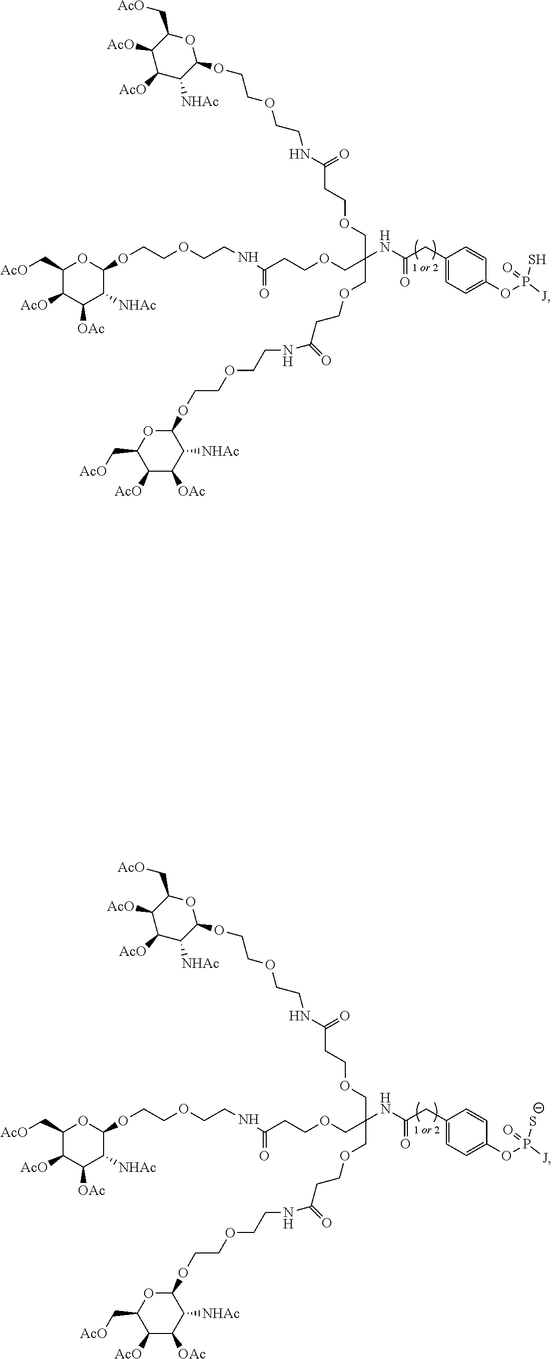

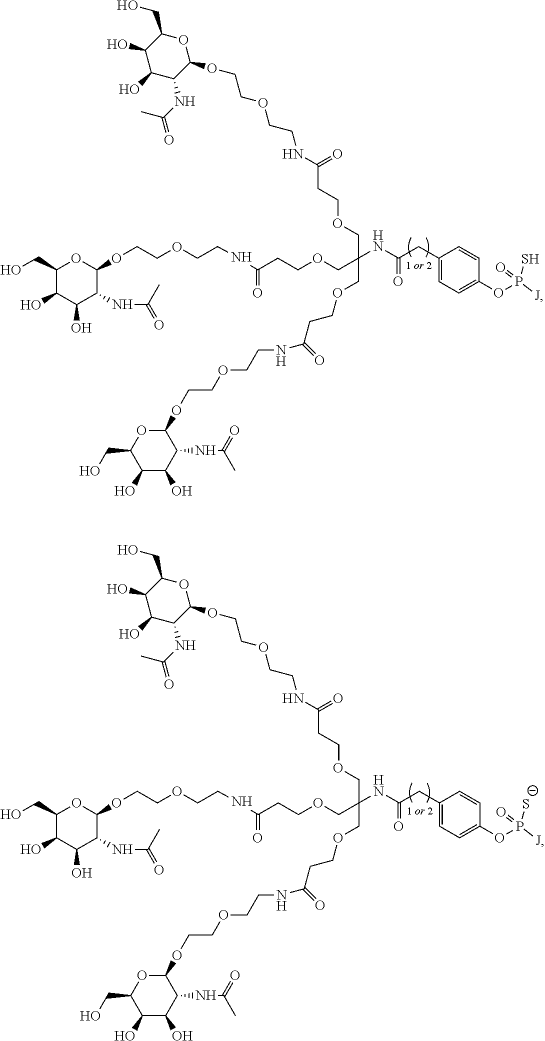

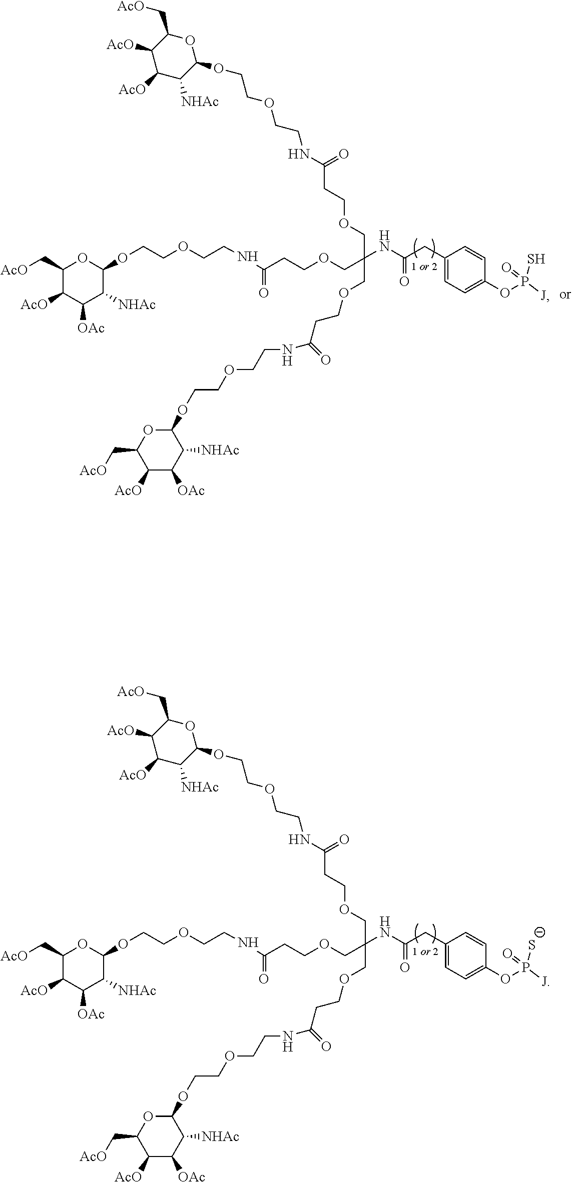

In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the compound binds to an asialoglycoprotein receptor. In some embodiments, the compound targets a hepatocyte.

### F. siRNA Modification Patterns

In some embodiments, the composition comprises an oligonucleotide that inhibits the expression of SOS2 wherein the oligonucleotide comprises an siRNA comprising a sense strand and an antisense strand, wherein the sense strand comprises modification pattern 1S: 5′-NfsnsNfnNfnNfNfNfnNfnNfnNfnNfnNfsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 3S: 5′-nsnsnnNfnNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 5S: 5′-nsnsnnNfnNfNfNfnnnnnnnnnnsnsnN-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 6S: 5′-nnnnnnnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 7S: 5′-nnnnnnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 8S: 5′-nnnnnnNfnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 9S: 5′-nnnnnnNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 10S: 5′-nnnnnnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 11S: 5′-nnnnnnNfNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 12S: 5′-nnnnnNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 13S: 5′-nnnnnNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 14S: 5′-nnnnnNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 15S: 5′-nnnnnNfnNfNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 16S: 5′-nnnnnNfnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 17S: 5′-nnnnnNfNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 18S: 5′-nnnnnNfNfnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 19S: 5′-nnnnnNfNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 20S: 5′-nnnnnNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 21S: 5′-nnnnnNfNfNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 22S: 5′-nnnnnNfNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 23S: 5′-nnnnNfnnnNfhNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 24S: 5′-nnnnNfnnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 25S: 5′-nnnnNfnnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 26S: 5′-nnnnNfnnNfNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 27S: 5′-nnnnNfnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 28S: 5′-nnnnNfnNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 29S: 5′-nnnnNfnNfnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 30S: 5′-nnnnNfnNfnNfNfhnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 31S: 5′-nnnnNfnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 32S: 5′-nnnnNfNfnnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 33S: 5′-nnnnNfNfnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 34S: 5′-nnnnNfNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 35S: 5′-nnnnNfNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 36S: 5′-nnnnNfNfnNfNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 37S: 5′-nnnnNfNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 38S: 5′-nnnnnnnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 39S: 5′-nnnnnnnnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 40S: 5′-nnnnnnnnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 41S: 5′-snnnnnNfnNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 42S: 5′-snnnnNfnNfnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 43S: 5′-snnnnnnNfnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 44S: 5′-snnnnNfnNfNfdNNfnnnnnnnnnsnsn-3′, wherein “Nf” is a 2′ fluoro-modified nucleoside, “n” is a 2′ O-methyl modified nucleoside, “dN” comprises a deoxy nucleoside, and “s” is a phosphorothioate linkage. In some embodiments, the sense strand comprises modification pattern 45S: 5′-snnnnnNfnnNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 46S: 5′-snnnnNfNfNfNfNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 47S: 5′-snnnnNfnnNfNfNfNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 48S: 5′-snnnnNfNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 49S: 5′-snnnnNfnNfnNfNfhnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 50S: 5′-snnnnnNfNfNfNfnNfhnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 51 S: 5′-snnnnnNfnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 52S: 5′-snnnnnnNfNfNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 53S: 5′-snnnnNfnnnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 54S: 5′-snnnnnNfNfnNfNfnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 55S: 5′-snnnnNfnNfNfdNnNfnnnnnnnnsnsn-3′, wherein “Nf” is a 2′ fluoro-modified nucleoside, “n” is a 2′ O-methyl modified nucleoside, “dN” comprises a deoxy nucleoside, and “s” is a phosphorothioate linkage. In some embodiments, the sense strand comprises modification pattern 56S: 5′-snnnnNfnnnNfnNfnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 57S: 5′-snnnnNfNfnnNfnnnnnnnnnnsnsn-3′, wherein “Nf” is a 2′ fluoro-modified nucleoside, “n” is a 2′ O-methyl modified nucleoside, and “s” is a phosphorothioate linkage. In some embodiments, the sense strand comprises modification pattern 58S: 5′-snnnnNfnnNfNfnNfhnnnnnnnsnsn-3′, wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of SOS2 wherein the oligonucleotide comprises an siRNA comprising a sense strand and an antisense strand, wherein the antisense strand comprises modification pattern 1AS: 5′-nsNfsnNfnNfnNfnNfnnnNfnNfnNfnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: 5′-nsNfsnnnNfnNfNfnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: 5′-nsNfsnnnNfnnnnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: 5′-nsNfsnNfnNfnnnnnnnNfnNfnnnsnsn-3′, wherein “Nf” is a 2′ fluoro-modified nucleoside, “n” is a 2′ O-methyl modified nucleoside, and “s” is a phosphorothioate linkage. In some embodiments, the antisense strand comprises modification pattern 5AS: 5′-nsNfsnNfnNfnNfnNfnNfnNfnNfnNfnsnsn-3′, wherein “Nf” is a 2′ fluoro-modified nucleoside, “n” is a 2′ O-methyl modified nucleoside, and “s” is a phosphorothioate linkage.

In some embodiments, the composition comprises an oligonucleotide that inhibits the expression of SOS2 wherein the oligonucleotide comprises an siRNA comprising a sense strand and an antisense strand, wherein the sense strand comprises pattern 1S and the antisense strand comprises pattern 1AS, 2AS, 3AS, 4AS, 5AS. In some embodiments, the sense strand comprises pattern 2S and the antisense strand comprises pattern 1AS, 2AS, 3AS, 4AS, or 5AS. In some embodiments, the sense strand comprises pattern 3S and the antisense strand comprises pattern 1AS, 2AS, 3AS, 4AS, or 5AS. In some embodiments, the sense strand comprises pattern 4S and the antisense strand comprises pattern 1AS, 2AS, 3AS, 4AS, or 5AS. In some embodiments, the sense strand comprises pattern 5S and the antisense strand comprises pattern 1AS, 2AS, 3AS, 4AS, or 5AS. In some embodiments, the sense strand comprises pattern 6S and the antisense strand comprises pattern 1AS, 2AS, 3AS, 4AS, or 5AS. In some embodiments, the sense strand comprises pattern 7S and the antisense strand comprises pattern 1AS, 2AS, 3AS, 4AS, or 5AS. In some embodiments, the sense strand comprises pattern 8S and the antisense strand comprises pattern 1AS, 2AS, 3AS, 4AS, or 5AS. In some embodiments, the sense strand comprises pattern 9S and the antisense strand comprises pattern 1AS, 2AS, 3AS, 4AS, or 5AS. In some embodiments, the sense strand comprises pattern 10S and the antisense strand comprises pattern 1AS, 2AS, 3AS, 4AS, or 5AS. In some embodiments, the sense strand comprises pattern 11S and the antisense strand comprises pattern 1AS, 2AS, 3AS, 4AS, or 5AS. In some embodiments, the sense strand comprises pattern 12S and the antisense strand comprises pattern 1AS, 2AS, 3AS, 4AS, or 5AS. In some embodiments, the sense strand comprises pattern 13S and the antisense strand comprises pattern 1AS, 2AS, 3AS, 4AS, or 5AS. In some embodiments, the sense strand comprises pattern 14S and the antisense strand comprises pattern 1AS, 2AS, 3AS, 4AS, or 5AS. In some embodiments, the sense strand comprises pattern 15S and the antisense strand comprises pattern 1AS, 2AS, 3AS, 4AS, or 5AS. In some embodiments, the sense strand comprises pattern 16S and the antisense strand comprises pattern 1AS, 2AS, 3AS, 4AS, or 5AS. In some embodiments, the sense strand comprises pattern 17S and the antisense strand comprises pattern 1AS, 2AS, 3AS, 4AS, or 5AS. In some embodiments, the sense strand comprises pattern 18S and the antisense strand comprises pattern 1AS, 2AS, 3AS, 4AS, or 5AS. In some embodiments, the sense strand comprises pattern 19S and the antisense strand comprises pattern 1AS, 2AS, 3AS, 4AS, or 5AS. In some embodiments, the sense strand comprises pattern 20S and the antisense strand comprises pattern 1AS, 2AS, 3AS, 4AS, or 5AS. In some embodiments, the sense strand comprises pattern 21S and the antisense strand comprises pattern 1AS, 2AS, 3AS, 4AS, or 5AS. In some embodiments, the sense strand comprises pattern 22S and the antisense strand comprises pattern 1AS, 2AS, 3AS, 4AS, or 5AS. In some embodiments, the sense strand comprises pattern 23S and the antisense strand comprises pattern 1AS, 2AS, 3AS, 4AS, or 5AS. In some embodiments, the sense strand comprises pattern 24S and the antisense strand comprises pattern 1AS, 2AS, 3AS, 4AS, or 5AS. In some embodiments, the sense strand comprises pattern 25S and the antisense strand comprises pattern 1AS, 2AS, 3AS, 4AS, or 5AS. In some embodiments, the sense strand comprises pattern 26S and the antisense strand comprises pattern 1AS, 2AS, 3AS, 4AS, or 5AS. In some embodiments, the sense strand comprises pattern 27S and the antisense strand comprises pattern 1AS, 2AS, 3AS, 4AS, or 5AS. In some embodiments, the sense strand comprises pattern 28S and the antisense strand comprises pattern 1AS, 2AS, 3AS, 4AS, or 5AS. In some embodiments, the sense strand comprises pattern 29S and the antisense strand comprises pattern 1AS, 2AS, 3AS, 4AS, or 5AS. In some embodiments, the sense strand comprises pattern 30S and the antisense strand comprises pattern 1AS, 2AS, 3AS, 4AS, or 5AS. In some embodiments, the sense strand comprises pattern 31S and the antisense strand comprises pattern 1AS, 2AS, 3AS, 4AS, or 5AS. In some embodiments, the sense strand comprises pattern 32S and the antisense strand comprises pattern 1AS, 2AS, 3AS, 4AS, or 5AS. In some embodiments, the sense strand comprises pattern 33S and the antisense strand comprises pattern 1AS, 2AS, 3AS, 4AS, or 5AS. In some embodiments, the sense strand comprises pattern 34S and the antisense strand comprises pattern 1AS, 2AS, 3AS, 4AS, or 5AS. In some embodiments, the sense strand comprises pattern 35S and the antisense strand comprises pattern 1AS, 2AS, 3AS, 4AS, or 5AS. In some embodiments, the sense strand comprises pattern 36S and the antisense strand comprises pattern 1AS, 2AS, 3AS, 4AS, or 5AS. In some embodiments, the sense strand comprises pattern 37S and the antisense strand comprises pattern 1AS, 2AS, 3AS, 4AS, or 5AS. In some embodiments, the sense strand comprises pattern 38S and the antisense strand comprises pattern 1AS, 2AS, 3AS, 4AS, or 5AS. In some embodiments, the sense strand comprises pattern 39S and the antisense strand comprises pattern 1AS, 2AS, 3AS, 4AS, or 5AS. In some embodiments, the sense strand comprises pattern 40S and the antisense strand comprises pattern 1AS, 2AS, 3AS, 4AS, or 5AS. In some embodiments, the sense strand comprises pattern 41S and the antisense strand comprises pattern 1AS, 2AS, 3AS, 4AS, or 5AS. In some embodiments, the sense strand comprises pattern 42S and the antisense strand comprises pattern 1AS, 2AS, 3AS, 4AS, or 5AS. In some embodiments, the sense strand comprises pattern 43S and the antisense strand comprises pattern 1AS, 2AS, 3AS, 4AS, or 5AS. In some embodiments, the sense strand comprises pattern 44S and the antisense strand comprises pattern 1AS, 2AS, 3AS, 4AS, or 5AS. In some embodiments, the sense strand comprises pattern 45S and the antisense strand comprises pattern 1AS, 2AS, 3AS, 4AS, or 5AS. In some embodiments, the sense strand comprises pattern 46S and the antisense strand comprises pattern 1AS, 2AS, 3AS, 4AS, or 5AS. In some embodiments, the sense strand comprises pattern 47S and the antisense strand comprises pattern 1AS, 2AS, 3AS, 4AS, or 5AS. In some embodiments, the sense strand comprises pattern 48S and the antisense strand comprises pattern 1AS, 2AS, 3AS, 4AS, or 5AS. In some embodiments, the sense strand comprises pattern 49S and the antisense strand comprises pattern 1AS, 2AS, 3AS, 4AS, or 5AS. In some embodiments, the sense strand comprises pattern 50S and the antisense strand comprises pattern 1AS, 2AS, 3AS, 4AS, or 5AS. In some embodiments, the sense strand comprises pattern 51S and the antisense strand comprises pattern 1AS, 2AS, 3AS, 4AS, or 5AS. In some embodiments, the sense strand comprises pattern 52S and the antisense strand comprises pattern 1AS, 2AS, 3AS, 4AS, or 5AS. In some embodiments, the sense strand comprises pattern 53S and the antisense strand comprises pattern 1AS, 2AS, 3AS, 4AS, or 5AS. In some embodiments, the sense strand comprises pattern 54S and the antisense strand comprises pattern 1AS, 2AS, 3AS, 4AS, or 5AS. In some embodiments, the sense strand comprises pattern 55S and the antisense strand comprises pattern 1AS, 2AS, 3AS, 4AS, or 5AS. In some embodiments, the sense strand comprises pattern 56S and the antisense strand comprises pattern 1AS, 2AS, 3AS, 4AS, or 5AS. In some embodiments, the sense strand comprises pattern 57S and the antisense strand comprises pattern 1AS, 2AS, 3AS, 4AS, or 5AS. In some embodiments, the sense strand comprises pattern 58S and the antisense strand comprises pattern 1AS, 2AS, 3AS, 4AS, or 5AS. In some embodiments, the sense strand comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 39S, or 40S. In some embodiments, the sense strand comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 39S, 40S, 41S, 42S, 43S, 44S, 45S, 46S, 47S, 48S, 49S, 50S, 51S, 52S, 53S, 54S, 55S, 56S, 57S, or 58S. In some embodiments, the sense strand comprises modification pattern 1AS, 2AS, 3AS, 4AS, or 5AS. In some embodiments, the antisense strand comprises modification pattern 1AS, 2AS, 3AS, 4AS, or 5AS. In some embodiments, the antisense strand comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 39S, 40S, 41S, 42S, 43S, 44S, 45S, 46S, 47S, 48S, 49S, 50S, 51S, 52S, 53S, 54S, 55S, 56S, 57S, or 58S. In some embodiments, the sense strand or the antisense strand comprises modification pattern ASO1.

The oligonucleotide may include purines. Examples of purines include adenine (A) or guanine (G), or modified versions thereof. The oligonucleotide may include pyrimidines. Examples of pyrimidines include cytosine (C), thymine (T), or uracil (U), or modified versions thereof.

In some embodiments, purines of the oligonucleotide comprise 2′-fluoro modified purines. In some embodiments, purines of the oligonucleotide comprise 2′-O-methyl modified purines. In some embodiments, purines of the oligonucleotide comprise a mixture of 2′-fluoro and 2′-O-methyl modified purines. In some embodiments, all purines of the oligonucleotide comprise 2′-fluoro modified purines. In some embodiments, all purines of the oligonucleotide comprise 2′-O-methyl modified purines. In some embodiments, all purines of the oligonucleotide comprise a mixture of 2′-fluoro and 2′-O-methyl modified purines.

In some embodiments, pyrimidines of the oligonucleotide comprise 2′-fluoro modified pyrimidines. In some embodiments, pyrimidines of the oligonucleotide comprise 2′-O-methyl modified pyrimidines. In some embodiments, pyrimidines of the oligonucleotide comprise a mixture of 2′-fluoro and 2′-O-methyl modified pyrimidines. In some embodiments, all pyrimidines of the oligonucleotide comprise 2′-fluoro modified pyrimidines. In some embodiments, all pyrimidines of the oligonucleotide comprise 2′-O-methyl modified pyrimidines. In some embodiments, all pyrimidines of the oligonucleotide comprise a mixture of 2′-fluoro and 2′-O-methyl modified pyrimidines.

In some embodiments, purines of the oligonucleotide comprise 2′-fluoro modified purines, and pyrimidines of the oligonucleotide comprise a mixture of 2′-fluoro and 2′-O-methyl modified pyrimidines. In some embodiments, purines of the oligonucleotide comprise 2′-O-methyl modified purines, and pyrimidines of the oligonucleotide comprise a mixture of 2′-fluoro and 2′-O-methyl modified pyrimidines. In some embodiments, purines of the oligonucleotide comprise 2′-fluoro modified purines, and pyrimidines of the oligonucleotide comprise 2′-O-methyl modified pyrimidines. In some embodiments, purines of the oligonucleotide comprise 2′-O-methyl modified purines, and pyrimidines of the oligonucleotide comprise 2′-fluoro modified pyrimidines. In some embodiments, pyrimidines of the oligonucleotide comprise 2′-fluoro modified pyrimidines, and purines of the oligonucleotide comprise a mixture of 2′-fluoro and 2′-O-methyl modified purines. In some embodiments, pyrimidines of the oligonucleotide comprise 2′-O-methyl modified pyrimidines, and purines of the oligonucleotide comprise a mixture of 2′-fluoro and 2′-O-methyl modified purines. In some embodiments, pyrimidines of the oligonucleotide comprise 2′-fluoro modified pyrimidines, and purines of the oligonucleotide comprise 2′-O-methyl modified purines. In some embodiments, pyrimidines of the oligonucleotide comprise 2′-O-methyl modified pyrimidines, and purines of the oligonucleotide comprise 2′-fluoro modified purines.

In some embodiments, all purines of the oligonucleotide comprise 2′-fluoro modified purines, and all pyrimidines of the oligonucleotide comprise a mixture of 2′-fluoro and 2′-O-methyl modified pyrimidines. In some embodiments, all purines of the oligonucleotide comprise 2′-O-methyl modified purines, and all pyrimidines of the oligonucleotide comprise a mixture of 2′-fluoro and 2′-O-methyl modified pyrimidines. In some embodiments, all purines of the oligonucleotide comprise 2′-fluoro modified purines, and all pyrimidines of the oligonucleotide comprise 2′-O-methyl modified pyrimidines. In some embodiments, all purines of the oligonucleotide comprise 2′-O-methyl modified purines, and all pyrimidines of the oligonucleotide comprise 2′-fluoro modified pyrimidines. In some embodiments, all pyrimidines of the oligonucleotide comprise 2′-fluoro modified pyrimidines, and all purines of the oligonucleotide comprise a mixture of 2′-fluoro and 2′-O-methyl modified purines. In some embodiments, all pyrimidines of the oligonucleotide comprise 2′-O-methyl modified pyrimidines, and all purines of the oligonucleotide comprise a mixture of 2′-fluoro and 2′-O-methyl modified purines. In some embodiments, all pyrimidines of the oligonucleotide comprise 2′-fluoro modified pyrimidines, and all purines of the oligonucleotide comprise 2′-O-methyl modified purines. In some embodiments, all pyrimidines of the oligonucleotide comprise 2′-O-methyl modified pyrimidines, and all purines of the oligonucleotide comprise 2′-fluoro modified purines.

In some embodiments, position nine of the sense strand comprises a 2′ fluoro-modified pyrimidine. In some embodiments, all purines of the sense strand comprise 2′-O-methyl modified purines. In some embodiments, 1, 2, 3, 4, or 5 pyrimidines between positions 5 and 11 comprise a 2′flouro-modified pyrimidine, provided there are never three 2′ fluoro-modified pyrimidines in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, position nine of the sense strand comprises a 2′ fluoro-modified pyrimidine; all purines of the sense strand comprises 2′-O-methyl modified purines; 1, 2, 3, 4, or 5 pyrimidines between positions 5 and 11 comprise a 2′flouro-modified pyrimidine, provided there are never three 2′ fluoro-modified pyrimidines in a row; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises a 2′ fluoro-modified purine. In some embodiments, all pyrimidines of the sense strand comprise 2′-O-methyl modified purines. In some embodiments, 1, 2, 3, 4, or 5 purines between positions 5 and 11 comprise a 2′flouro-modified purine, provided there are never three 2′ fluoro-modified purine in a row. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotide. In some embodiments, position nine of the sense strand comprises a 2′ fluoro-modified purine; all pyrimidine of the sense strand comprises 2′-O-methyl modified pyrimidines; 1, 2, 3, 4, or 5 purines between positions 5 and 11 comprise a 2′flouro-modified purines, provided there are never three 2′ fluoro-modified purines in a row; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2′fluoro-modified nucleotides. In some embodiments, all pyrimidines in positions 10 to 21 of the sense strand comprise 2′-O-methyl modified pyrimidines and all purines in positions 10 to 21 of the comprise 2′-O-methyl modified purines or 2′fluoro-modified purines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all pyrimidines in positions 10 to 21 of the sense strand comprise 2′-O-methyl modified pyrimidines and all purines in positions 10 to 21 of the comprise 2′-O-methyl modified purines or 2′fluoro-modified purines; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, position nine of the sense strand comprises an unmodified deoxyribonucleotide. In some embodiments, positions 5, 7, and 8 of the sense strand comprise 2′fluoro-modified nucleotides. In some embodiments, all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines. In some embodiments, the odd-numbered positions of the antisense strand comprise 2′-O-methyl modified nucleotides. In some embodiments, the even-numbered positions of the antisense strand comprise 2′flouro-modified nucleotides and unmodified deoxyribonucleotides. In some embodiments, position nine of the sense strand comprises an unmodified deoxyribonucleotide; positions 5, 7, and 8 of the sense strand comprise 2′fluoro-modified nucleotides; all purines in positions 10 to 21 of the sense strand comprise 2′-O-methyl modified purines and all pyrimidines in positions 10 to 21 of the comprise 2′-O-methyl modified pyrimidines or 2′fluoro-modified pyrimidines; the odd-numbered positions of the antisense strand comprise 2′-O-methyl modified nucleotides; and the even-numbered positions of the antisense strand comprise 2′flouro-modified nucleotides and unmodified deoxyribonucleotides.

In some embodiments, the sense strand comprises or consists of a sequence at least 75% identical to of any one of SEQ ID NOs: 10981-11000, 11021-11115, 11091-11115, or 11141-11252, at least 80% identical to of any one of SEQ ID NOs: 10981-11000, 11021-11115, 11091-11115, or 11141-11252, at least 85% identical to of any one of SEQ ID NOs: 10981-11000, 11021-11115, 11091-11115, or 11141-11252, at least 90% identical to of any one of SEQ ID NOs: 10981-11000, 11021-11115, 11091-11115, or 11141-11252, or at least 95% identical to of any one of SEQ ID NOs: 10981-11000, 11021-11115, 11091-11115, or 11141-11252. In some embodiments, the sense strand comprises or consists of the sequence of any one of SEQ ID NOs: 10981-11000, 11021-11115, 11091-11115, or 11141-11252, or a sequence thereof having 1, 2, 3, or 4 nucleoside substitutions, additions, or deletions. In some embodiments, the sense strand comprises or consists of the sequence of any one of SEQ ID NOs: 10981-11000, 11021-11115, 11091-11115, or 11141-11252, or a sequence thereof having 1 or 2 nucleoside substitutions, additions, or deletions. In some embodiments, the sense strand comprises or consists of the sequence of any one of SEQ ID NOs: 10981-11000, 11021-11115, 11091-11115, or 11141-11252. In some embodiments, the sense strand is an unmodified version of a nucleic acid sequence described herein. In some embodiments, the sense strand has more or different sequence modifications than a nucleic acid sequence described herein.

In some embodiments, the antisense strand comprises or consists of a sequence at least 75% identical to of any one of SEQ ID NOs: 11001-11020 or 11116-11140, at least 80% identical to of any one of SEQ ID NOs: 11001-11020 or 11116-11140, at least 85% identical to of any one of SEQ ID NOs: 11001-11020 or 11116-11140, at least 90% identical to of any one of SEQ ID NOs: 11001-11020 or 11116-11140, or at least 95% identical to of any one of SEQ ID NOs: 11001-11020 or 11116-11140. In some embodiments, the antisense strand comprises or consists of the sequence of any one of SEQ ID NOs: 11001-11020 or 11116-11140 or a sequence thereof having 1, 2, 3, or 4 nucleoside substitutions, additions, or deletions. In some embodiments, the antisense strand comprises or consists of the sequence of any one of SEQ ID NOs: 11001-11020 or 11116-11140, or a sequence thereof having 1 or 2 nucleoside substitutions, additions, or deletions. In some embodiments, the antisense strand comprises or consists of the sequence of any one of SEQ ID NOs: 11001-11020 or 11116-11140. In some embodiments, the antisense strand is an unmodified version of a nucleic acid sequence described herein. In some embodiments, the antisense strand has more or different sequence modifications than a nucleic acid sequence described herein.

In some embodiments, the sense strand sequence comprises or consists of a sense strand sequence of any one of the siRNAs disclosed in Tables 15-25, or an siRNA thereof having 1 or 2 nucleoside substitutions, additions, or deletions. In some embodiments, the sense strand sequence comprises or consists of a sense strand sequence of any one of the siRNAs disclosed in Tables 15-25. In some embodiments, the sense strand sequence comprises or consists of a sense strand sequence of any one of the siRNAs disclosed in Table 15, or an siRNA thereof having 1 or 2 nucleoside substitutions, additions, or deletions. In some embodiments, the sense strand sequence comprises or consists of a sense strand sequence of any one of the siRNAs disclosed in Table 15. In some embodiments, the sense strand sequence comprises or consists of a sense strand sequence of any one of the siRNAs disclosed in Table 16, or an siRNA thereof having 1 or 2 nucleoside substitutions, additions, or deletions. In some embodiments, the sense strand sequence comprises or consists of a sense strand sequence of any one of the siRNAs disclosed in Table 16. In some embodiments, the sense strand sequence comprises or consists of a sense strand sequence of any one of the siRNAs disclosed in Table 17, or an siRNA thereof having 1 or 2 nucleoside substitutions, additions, or deletions. In some embodiments, the sense strand sequence comprises or consists of a sense strand sequence of any one of the siRNAs disclosed in Table 17. In some embodiments, the sense strand sequence comprises or consists of a sense strand sequence of any one of the siRNAs disclosed in Table 18, or an siRNA thereof having 1 or 2 nucleoside substitutions, additions, or deletions. In some embodiments, the sense strand sequence comprises or consists of a sense strand sequence of any one of the siRNAs disclosed in Table 18. In some embodiments, the sense strand sequence comprises or consists of a sense strand sequence of any one of the siRNAs disclosed in Table 19, or an siRNA thereof having 1 or 2 nucleoside substitutions, additions, or deletions. In some embodiments, the sense strand sequence comprises or consists of a sense strand sequence of any one of the siRNAs disclosed in Table 19. In some embodiments, the sense strand sequence comprises or consists of a sense strand sequence of any one of the siRNAs disclosed in Table 20, or an siRNA thereof having 1 or 2 nucleoside substitutions, additions, or deletions. In some embodiments, the sense strand sequence comprises or consists of a sense strand sequence of any one of the siRNAs disclosed in Table 20. In some embodiments, the sense strand sequence comprises or consists of a sense strand sequence of any one of the siRNAs disclosed in Table 21, or an siRNA thereof having 1 or 2 nucleoside substitutions, additions, or deletions. In some embodiments, the sense strand sequence comprises or consists of a sense strand sequence of any one of the siRNAs disclosed in Table 21. In some embodiments, the sense strand sequence comprises or consists of a sense strand sequence of any one of the siRNAs disclosed in Table 22, or an siRNA thereof having 1 or 2 nucleoside substitutions, additions, or deletions. In some embodiments, the sense strand sequence comprises or consists of a sense strand sequence of any one of the siRNAs disclosed in Table 22. In some embodiments, the sense strand sequence comprises or consists of a sense strand sequence of any one of the siRNAs disclosed in Table 23, or an siRNA thereof having 1 or 2 nucleoside substitutions, additions, or deletions. In some embodiments, the sense strand sequence comprises or consists of a sense strand sequence of any one of the siRNAs disclosed in Table 23. In some embodiments, the sense strand sequence comprises or consists of a sense strand sequence of any one of the siRNAs disclosed in Table 24, or an siRNA thereof having 1 or 2 nucleoside substitutions, additions, or deletions. In some embodiments, the sense strand sequence comprises or consists of a sense strand sequence of any one of the siRNAs disclosed in Table 24. In some embodiments, the sense strand sequence comprises or consists of a sense strand sequence of any one of the siRNAs disclosed in Table 25, or an siRNA thereof having 1 or 2 nucleoside substitutions, additions, or deletions. In some embodiments, the sense strand sequence comprises or consists of a sense strand sequence of any one of the siRNAs disclosed in Table 25.

In some embodiments, the antisense strand sequence comprises or consists of an antisense strand sequence of any one of the siRNAs disclosed in Tables 15-25, or an siRNA thereof having 1 or 2 nucleoside substitutions, additions, or deletions. In some embodiments, the antisense strand sequence comprises or consists of an antisense strand sequence of any one of the siRNAs disclosed in Tables 15-25. In some embodiments, the antisense strand sequence comprises or consists of an antisense strand sequence of any one of the siRNAs disclosed in Table 15, or an siRNA thereof having 1 or 2 nucleoside substitutions, additions, or deletions. In some embodiments, the antisense strand sequence comprises or consists of an antisense strand sequence of any one of the siRNAs disclosed in Table 15. In some embodiments, the antisense strand sequence comprises or consists of an antisense strand sequence of any one of the siRNAs disclosed in Table 16, or an siRNA thereof having 1 or 2 nucleoside substitutions, additions, or deletions. In some embodiments, the antisense strand sequence comprises or consists of an antisense strand sequence of any one of the siRNAs disclosed in Table 16. In some embodiments, the antisense strand sequence comprises or consists of an antisense strand sequence of any one of the siRNAs disclosed in Table 17, or an siRNA thereof having 1 or 2 nucleoside substitutions, additions, or deletions. In some embodiments, the antisense strand sequence comprises or consists of an antisense strand sequence of any one of the siRNAs disclosed in Table 17. In some embodiments, the antisense strand sequence comprises or consists of an antisense strand sequence of any one of the siRNAs disclosed in Table 18, or an siRNA thereof having 1 or 2 nucleoside substitutions, additions, or deletions. In some embodiments, the antisense strand sequence comprises or consists of an antisense strand sequence of any one of the siRNAs disclosed in Table 18. In some embodiments, the antisense strand sequence comprises or consists of an antisense strand sequence of any one of the siRNAs disclosed in Table 19, or an siRNA thereof having 1 or 2 nucleoside substitutions, additions, or deletions. In some embodiments, the antisense strand sequence comprises or consists of an antisense strand sequence of any one of the siRNAs disclosed in Table 19. In some embodiments, the antisense strand sequence comprises or consists of an antisense strand sequence of any one of the siRNAs disclosed in Table 20, or an siRNA thereof having 1 or 2 nucleoside substitutions, additions, or deletions. In some embodiments, the antisense strand sequence comprises or consists of an antisense strand sequence of any one of the siRNAs disclosed in Table 20. In some embodiments, the antisense strand sequence comprises or consists of an antisense strand sequence of any one of the siRNAs disclosed in Table 21, or an siRNA thereof having 1 or 2 nucleoside substitutions, additions, or deletions. In some embodiments, the antisense strand sequence comprises or consists of an antisense strand sequence of any one of the siRNAs disclosed in Table 21. In some embodiments, the antisense strand sequence comprises or consists of an antisense strand sequence of any one of the siRNAs disclosed in Table 22, or an siRNA thereof having 1 or 2 nucleoside substitutions, additions, or deletions. In some embodiments, the antisense strand sequence comprises or consists of an antisense strand sequence of any one of the siRNAs disclosed in Table 22. In some embodiments, the antisense strand sequence comprises or consists of an antisense strand sequence of any one of the siRNAs disclosed in Table 23, or an siRNA thereof having 1 or 2 nucleoside substitutions, additions, or deletions. In some embodiments, the antisense strand sequence comprises or consists of an antisense strand sequence of any one of the siRNAs disclosed in Table 23. In some embodiments, the antisense strand sequence comprises or consists of an antisense strand sequence of any one of the siRNAs disclosed in Table 24, or an siRNA thereof having 1 or 2 nucleoside substitutions, additions, or deletions. In some embodiments, the antisense strand sequence comprises or consists of an antisense strand sequence of any one of the siRNAs disclosed in Table 24. In some embodiments, the antisense strand sequence comprises or consists of an antisense strand sequence of any one of the siRNAs disclosed in Table 25, or an siRNA thereof having 1 or 2 nucleoside substitutions, additions, or deletions. In some embodiments, the antisense strand sequence comprises or consists of an antisense strand sequence of any one of the siRNAs disclosed in Table 25.

In some embodiments, the sense strand includes a nucleoside sequence of a sense strand in any of Tables 21-25 and omits an A, U, UU, or AUU. For example, a sense strand may omit a 3′ AUU of a sense strand sequence in any of Tables 21-25. In some embodiments, the sense strand comprises the nucleoside sequence of positions 1-18 of a sense strand sequence in any of Tables 21-25. In some embodiments, the sense strand comprises a sequence at least 80%, at least 85%, at least 90%, or at least 95% identical to the nucleoside sequence of positions 1-18 of a sense strand sequence in any of Tables 21-25. In some embodiments, the sense strand comprises the nucleoside sequence of positions 1-18 of a sense strand sequence in any of Tables 21-25, or a sequence comprising 1 or 2 nucleoside substitutions, additions, or deletions. In some embodiments, the sense strand comprises the nucleoside sequence of positions 1-18 of a sense strand sequence in any of Tables 21-25, or a sequence comprising 3 or 4 nucleoside substitutions, additions, or deletions.

A sense strand sequence may omit a 3′ AUU of a sense strand sequence in Table 21. In some embodiments, the sense strand comprises the nucleoside sequence of positions 1-18 of a sense strand in Table 21. In some embodiments, the sense strand comprises the nucleoside sequence of positions 1-19 of a sense strand in Table 21. In some embodiments, the sense strand comprises the nucleoside sequence of positions 1-20 of a sense strand of Table 21. In some embodiments, the sense strand comprises a sequence that is at least 90% identical to a nucleoside sequence in Table 21 omitting an AUU sequence. In some embodiments, the sense strand can be a nucleoside sequence that is at least 90% identical to a nucleoside sequence in Table 21 and omits at least one nucleoside comprising an A, a U, a UU, or an AUU.

A sense strand sequence may omit a 3′ AUU of a sense strand sequence in Table 22. In some embodiments, the sense strand comprises the nucleoside sequence of positions 1-18 of a sense strand in Table 22. In some embodiments, the sense strand comprises the nucleoside sequence of positions 1-19 of a sense strand in Table 22. In some embodiments, the sense strand comprises the nucleoside sequence of positions 1-20 of a sense strand of Table 22. In some embodiments, the sense strand comprises a sequence that is at least 90% identical to a nucleoside sequence in Table 22 omitting an AUU sequence. In some embodiments, the sense strand can be a nucleoside sequence that is at least 90% identical to a nucleoside sequence in Table 22 and omits at least one nucleoside comprising an A, a U, a UU, or an AUU.

A sense strand sequence may omit a 3′ AUU of a sense strand sequence in Table 23. In some embodiments, the sense strand comprises the nucleoside sequence of positions 1-18 of a sense strand in Table 23. In some embodiments, the sense strand comprises the nucleoside sequence of positions 1-19 of a sense strand in Table 23. In some embodiments, the sense strand comprises the nucleoside sequence of positions 1-20 of a sense strand of Table 23. In some embodiments, the sense strand comprises a sequence that is at least 90% identical to a nucleoside sequence in Table 23 omitting an AUU sequence. In some embodiments, the sense strand can be a nucleoside sequence that is at least 90% identical to a nucleoside sequence in Table 23 and omits at least one nucleoside comprising an A, a U, a UU, or an AUU.

A sense strand sequence may omit a 3′ AUU of a sense strand sequence in Table 24. In some embodiments, the sense strand comprises the nucleoside sequence of positions 1-18 of a sense strand in Table 24. In some embodiments, the sense strand comprises the nucleoside sequence of positions 1-19 of a sense strand in Table 24. In some embodiments, the sense strand comprises the nucleoside sequence of positions 1-20 of a sense strand of Table 24. In some embodiments, the sense strand comprises a sequence that is at least 90% identical to a nucleoside sequence in Table 24 omitting an AUU sequence. In some embodiments, the sense strand can be a nucleoside sequence that is at least 90% identical to a nucleoside sequence in Table 24 and omits at least one nucleoside comprising an A, a U, a UU, or an AUU.

A sense strand sequence may omit a 3′ AUU of a sense strand sequence in Table 25. In some embodiments, the sense strand comprises the nucleoside sequence of positions 1-18 of a sense strand in Table 25. In some embodiments, the sense strand comprises the nucleoside sequence of positions 1-19 of a sense strand in Table 25. In some embodiments, the sense strand comprises the nucleoside sequence of positions 1-20 of a sense strand of Table 25. In some embodiments, the sense strand comprises a sequence that is at least 90% identical to a nucleoside sequence in Table 25 omitting an AUU sequence. In some embodiments, the sense strand can be a nucleoside sequence that is at least 90% identical to a nucleoside sequence in Table 25 and omits at least one nucleoside comprising an A, a U, a UU, or an AUU.

In some embodiments, the antisense strand includes a nucleoside sequence of an antisense strand in any of Tables 21-25 and omits a U or UU. For example, an antisense strand may omit a 5′ U and a 3′ UU of an antisense strand sequence in any of Tables 21-25. In some embodiments, the antisense strand comprises the nucleoside sequence of positions 2-19 of an antisense strand sequence in any of Tables 21-25. In some embodiments, the antisense strand comprises a sequence at least 80%, at least 85%, at least 90%, or at least 95% identical to the nucleoside sequence of positions 2-19 of an antisense strand sequence in any of Tables 21-25. In some embodiments, the antisense strand comprises the nucleoside sequence of positions 2-19 of an antisense strand sequence in any of Tables 21-25, or a sequence comprising 1 or 2 nucleoside substitutions, additions, or deletions. In some embodiments, the antisense strand comprises the nucleoside sequence of positions 2-19 of an antisense strand sequence in any of Tables 21-25, or a sequence comprising 3 or 4 nucleoside substitutions, additions, or deletions.

An antisense strand sequence may omit a 5′ U and 3′ UU of an antisense strand sequence in Table 21. In some embodiments, the antisense strand comprises the nucleoside sequence of positions 1-18 of an antisense strand in Table 21. In some embodiments, the antisense strand comprises the nucleoside sequence of positions 2-19 of an antisense strand in Table 21. In some embodiments, the antisense strand comprises the nucleoside sequence of positions 2-21 of an antisense strand of Table 21. In some embodiments, the antisense strand comprises a sequence that is at least 90% identical to a nucleoside sequence in Table 21 omitting a 5′ U and a 3′ UU. In some embodiments, the antisense strand can be a nucleoside sequence that is at least 90% identical to a nucleoside sequence in Table 21 and omits at least one nucleoside comprising a U or UU.

An antisense strand sequence may omit a 5′ U and 3′ UU of an antisense strand sequence in Table 22. In some embodiments, the antisense strand comprises the nucleoside sequence of positions 1-18 of an antisense strand in Table 22. In some embodiments, the antisense strand comprises the nucleoside sequence of positions 2-19 of an antisense strand in Table 22. In some embodiments, the antisense strand comprises the nucleoside sequence of positions 2-21 of an antisense strand of Table 22. In some embodiments, the antisense strand comprises a sequence that is at least 90% identical to a nucleoside sequence in Table 22 omitting a 5′ U and a 3′ UU. In some embodiments, the antisense strand can be a nucleoside sequence that is at least 90% identical to a nucleoside sequence in Table 22 and omits at least one nucleoside comprising a U or UU.

An antisense strand sequence may omit a 5′ U and 3′ UU of an antisense strand sequence in Table 23. In some embodiments, the antisense strand comprises the nucleoside sequence of positions 1-18 of an antisense strand in Table 23. In some embodiments, the antisense strand comprises the nucleoside sequence of positions 2-19 of an antisense strand in Table 23. In some embodiments, the antisense strand comprises the nucleoside sequence of positions 2-21 of an antisense strand of Table 23. In some embodiments, the antisense strand comprises a sequence that is at least 90% identical to a nucleoside sequence in Table 23 omitting a 5′ U and a 3′ UU. In some embodiments, the antisense strand can be a nucleoside sequence that is at least 90% identical to a nucleoside sequence in Table 23 and omits at least one nucleoside comprising a U or UU.

An antisense strand sequence may omit a 5′ U and 3′ UU of an antisense strand sequence in Table 24. In some embodiments, the antisense strand comprises the nucleoside sequence of positions 1-18 of an antisense strand in Table 24. In some embodiments, the antisense strand comprises the nucleoside sequence of positions 2-19 of an antisense strand in Table 24. In some embodiments, the antisense strand comprises the nucleoside sequence of positions 2-21 of an antisense strand of Table 24. In some embodiments, the antisense strand comprises a sequence that is at least 90% identical to a nucleoside sequence in Table 24 omitting a 5′ U and a 3′ UU. In some embodiments, the antisense strand can be a nucleoside sequence that is at least 90% identical to a nucleoside sequence in Table 24 and omits at least one nucleoside comprising a U or UU.

An antisense strand sequence may omit a 5′ U and 3′ UU of an antisense strand sequence in Table 25. In some embodiments, the antisense strand comprises the nucleoside sequence of positions 1-18 of an antisense strand in Table 25. In some embodiments, the antisense strand comprises the nucleoside sequence of positions 2-19 of an antisense strand in Table 25. In some embodiments, the antisense strand comprises the nucleoside sequence of positions 2-21 of an antisense strand of Table 25. In some embodiments, the antisense strand comprises a sequence that is at least 90% identical to a nucleoside sequence in Table 25 omitting a 5′ U and a 3′ UU. In some embodiments, the antisense strand can be a nucleoside sequence that is at least 90% identical to a nucleoside sequence in Table 25 and omits at least one nucleoside comprising a U or UU.

In some embodiments, the siRNA comprises a sense strand or antisense strand having a sequence in accordance with the sense strand or antisense strand sequence of an siRNA in Table 27. In some embodiments, the sense strand or antisense strand comprises a sequence at least 750% identical, at least 80% identical, at least 85% identical, at least 90% identical, or at least 95% identical, to a sense strand or antisense strand sequence in Table 27. In some embodiments, the sense strand or antisense strand comprises a sequence of a sense or antisense strand in Table 27, or a sequence thereof having 3 or 4 nucleoside substitutions, additions, or deletions. In some embodiments, the sense strand or antisense strand comprises a sequence of a sense or antisense strand in Table 27, or a sequence thereof having 1 or 2 nucleoside substitutions, additions, or deletions. In some embodiments, the sense strand or antisense sequence comprises or consists of a sequence 100% identical to a sense strand or antisense strand sequence in Table 27. The sense strand or antisense strand may comprise any modifications described herein. The sense strand or antisense strand may comprise a lipid moiety or a GalNAc moiety.

In some embodiments, the siRNA comprises a sense strand or antisense strand having a sequence in accordance with the sense strand or antisense strand sequence of an siRNA in Table 31. In some embodiments, the sense strand or antisense strand comprises a sequence at least 75% identical, at least 80% identical, at least 85% identical, at least 90% identical, or at least 95% identical, to a sense strand or antisense strand sequence in Table 31. In some embodiments, the sense strand or antisense strand comprises a sequence of a sense or antisense strand in Table 31, or a sequence thereof having 3 or 4 nucleoside substitutions, additions, or deletions. In some embodiments, the sense strand or antisense strand comprises a sequence of a sense or antisense strand in Table 31, or a sequence thereof having 1 or 2 nucleoside substitutions, additions, or deletions. In some embodiments, the sense strand or antisense sequence comprises or consists of a sequence 100% identical to a sense strand or antisense strand sequence in Table 31. The sense strand or antisense strand may comprise any modifications described herein. The sense strand or antisense strand may comprise a lipid moiety or a GalNAc moiety.

In some embodiments, the siRNA comprises a sense strand having a sequence in accordance with any of SEQ ID NOs: 10981-11000. In some embodiments, the sense strand sequence comprises or consists of sequence at least 75% identical to any one of SEQ ID NOs: 10981-11000, at least 80% identical to any one of SEQ ID NOs: 10981-11000, at least 85% identical to of any one of SEQ ID NOs: 10981-11000, at least 90% identical to any one of SEQ ID NOs: 1-5490, or at least 95% identical to any one of SEQ ID NOs: 10981-11000. In some embodiments, the sense strand sequence comprises or consists of the sequence of any one of SEQ ID NOs: 10981-11000, or a sense strand sequence thereof having 1, 2, 3, or 4 nucleoside substitutions, additions, or deletions. In some embodiments, the sense strand sequence comprises or consists of the sequence of any one of SEQ ID NOs: 10981-11000, or a sense strand sequence thereof having 1 or 2 nucleoside substitutions, additions, or deletions. In some embodiments, the sense strand sequence comprises or consists of a sequence 100% identical to SEQ ID NOs: 10981-11000. The sense strand may comprise a modification pattern described herein. The sense strand may comprise a lipid moiety. The sense strand may comprise a GalNAc moiety.

In some embodiments, the siRNA comprises an antisense strand having a sequence in accordance with any of SEQ ID NOs: 11001-11020. In some embodiments, the antisense strand sequence comprises or consists of sequence at least 75% identical to any one of SEQ ID NOs: 11001-11020, at least 80% identical to any one of SEQ ID NOs: 11001-11020, at least 85% identical to of any one of SEQ ID NOs: 11001-11020, at least 90% identical to any one of SEQ ID NOs: 11001-11020, or at least 95% identical to any one of SEQ ID NOs: 11001-11020. In some embodiments, the antisense strand sequence comprises or consists of the sequence of any one of SEQ ID NOs: 11001-11020, or an antisense strand sequence thereof having 1, 2, 3, or 4 nucleoside substitutions, additions, or deletions. In some embodiments, the antisense strand sequence comprises or consists of the sequence of any one of SEQ ID NOs: 11001-11020, or an antisense strand sequence thereof having 1 or 2 nucleoside substitutions, additions, or deletions. In some embodiments, the antisense strand sequence comprises or consists of a sequence 100% identical to SEQ ID NOs: 11001-11020. The antisense strand may comprise a modification pattern described herein. The antisense strand may comprise a lipid moiety. The antisense strand may comprise a GalNAc moiety.

In some embodiments, the siRNA comprises a sense strand having a sequence in accordance with any of SEQ ID NOs: 11021-11090. In some embodiments, the sense strand sequence comprises or consists of sequence at least 75% identical to any one of SEQ ID NOs: 11021-11090, at least 80% identical to any one of SEQ ID NOs: 11021-11090, at least 85% identical to of any one of SEQ ID NOs: 11021-11090, at least 90% identical to any one of SEQ ID NOs: 1-5490, or at least 95% identical to any one of SEQ ID NOs: 11021-11090. In some embodiments, the sense strand sequence comprises or consists of the sequence of any one of SEQ ID NOs: 11021-11090, or a sense strand sequence thereof having 1, 2, 3, or 4 nucleoside substitutions, additions, or deletions. In some embodiments, the sense strand sequence comprises or consists of the sequence of any one of SEQ ID NOs: 11021-11090, or a sense strand sequence thereof having 1 or 2 nucleoside substitutions, additions, or deletions. In some embodiments, the sense strand sequence comprises or consists of a sequence 100% identical to SEQ ID NOs: 11021-11090. The sense strand may comprise a modification pattern described herein. The sense strand may comprise a lipid moiety. The sense strand may comprise a GalNAc moiety.

In some embodiments, the siRNA comprises a sense strand having a sequence in accordance with any of SEQ ID NOs: 11091-11115. In some embodiments, the sense strand sequence comprises or consists of sequence at least 75% identical to any one of SEQ ID NOs: 11091-11115, at least 80% identical to any one of SEQ ID NOs: 11091-11115, at least 85% identical to of any one of SEQ ID NOs: 11091-11115, at least 90% identical to any one of SEQ ID NOs: 1-5490, or at least 95% identical to any one of SEQ ID NOs: 11091-11115. In some embodiments, the sense strand sequence comprises or consists of the sequence of any one of SEQ ID NOs: 11091-11115, or a sense strand sequence thereof having 1, 2, 3, or 4 nucleoside substitutions, additions, or deletions. In some embodiments, the sense strand sequence comprises or consists of the sequence of any one of SEQ ID NOs: 11091-11115, or a sense strand sequence thereof having 1 or 2 nucleoside substitutions, additions, or deletions. In some embodiments, the sense strand sequence comprises or consists of a sequence 100% identical to SEQ ID NOs: 11091-11115. The sense strand may comprise a modification pattern described herein. The sense strand may comprise a lipid moiety such as a cholesterol moiety described herein.

In some embodiments, the siRNA comprises an antisense strand having a sequence in accordance with any of SEQ ID NOs: 11116-11140. In some embodiments, the antisense strand sequence comprises or consists of sequence at least 75% identical to any one of SEQ ID NOs: 11116-11140, at least 80% identical to any one of SEQ ID NOs: 11116-11140, at least 85% identical to of any one of SEQ ID NOs: 11116-11140, at least 90% identical to any one of SEQ ID NOs: 11116-11140, or at least 95% identical to any one of SEQ ID NOs: 11116-11140. In some embodiments, the antisense strand sequence comprises or consists of the sequence of any one of SEQ ID NOs: 11116-11140, or an antisense strand sequence thereof having 1, 2, 3, or 4 nucleoside substitutions, additions, or deletions. In some embodiments, the antisense strand sequence comprises or consists of the sequence of any one of SEQ ID NOs: 11116-11140, or an antisense strand sequence thereof having 1 or 2 nucleoside substitutions, additions, or deletions. In some embodiments, the antisense strand sequence comprises or consists of a sequence 100% identical to SEQ ID NOs: 11116-11140. The antisense strand may comprise a modification pattern described herein. The antisense strand may comprise a lipid moiety. The antisense strand may comprise a GalNAc moiety.

In some embodiments, the siRNA comprises a sense strand having a sequence in accordance with any of SEQ ID NOs: 11141-11252. In some embodiments, the sense strand sequence comprises or consists of sequence at least 75% identical to any one of SEQ ID NOs: 11141-11252, at least 80% identical to any one of SEQ ID NOs: 11141-11252, at least 85% identical to of any one of SEQ ID NOs: 11141-11252, at least 90% identical to any one of SEQ ID NOs: 1-5490, or at least 95% identical to any one of SEQ ID NOs: 11141-11252. In some embodiments, the sense strand sequence comprises or consists of the sequence of any one of SEQ ID NOs: 11141-11252, or a sense strand sequence thereof having 1, 2, 3, or 4 nucleoside substitutions, additions, or deletions. In some embodiments, the sense strand sequence comprises or consists of the sequence of any one of SEQ ID NOs: 11141-11252, or a sense strand sequence thereof having 1 or 2 nucleoside substitutions, additions, or deletions. In some embodiments, the sense strand sequence comprises or consists of a sequence 100% identical to SEQ ID NOs: 11141-11252. The sense strand may comprise a modification pattern described herein. The sense strand may comprise a lipid moiety. The sense strand may comprise a GalNAc moiety.

In some embodiments, the siRNA comprises an antisense strand having a sequence in accordance with any of SEQ ID NOs: 11119-11140. In some embodiments, the antisense strand sequence comprises or consists of sequence at least 75% identical to any one of SEQ ID NOs: 11119-11140, at least 80% identical to any one of SEQ ID NOs: 11119-11140, at least 85% identical to of any one of SEQ ID NOs: 11119-11140, at least 90% identical to any one of SEQ ID NOs: 11119-11140, or at least 95% identical to any one of SEQ ID NOs: 11119-11140. In some embodiments, the antisense strand sequence comprises or consists of the sequence of any one of SEQ ID NOs: 11119-11140, or an antisense strand sequence thereof having 1, 2, 3, or 4 nucleoside substitutions, additions, or deletions. In some embodiments, the antisense strand sequence comprises or consists of the sequence of any one of SEQ ID NOs: 11119-11140, or an antisense strand sequence thereof having 1 or 2 nucleoside substitutions, additions, or deletions. In some embodiments, the antisense strand sequence comprises or consists of a sequence 100% identical to SEQ ID NOs: 11119-11140. The antisense strand may comprise a modification pattern described herein. The antisense strand may comprise a lipid moiety. The antisense strand may comprise a GalNAc moiety.

### G. ASO Modification Patterns

In some embodiments, the composition comprises an oligonucleotide that inhibits the expression of SOS2, wherein the oligonucleotide comprises an antisense oligonucleotide (ASO). In some embodiments, the ASO comprises modification pattern ASO1: 5′-nsnsnsnsnsdNsdNsdNsdNsdNsdNsdNsdNsdNsdNsnsnsnsnsn-3′, wherein “dN” is any deoxynucleotide, “n” is a 2′O-methyl or 2′O-methoxyethyl-modified nucleoside, and “s” is a phosphorothioate linkage. In some embodiments, the ASO comprises modification pattern 1S, 2S, 3S, 4S, 5S, 6S, 7S, 8S, 9S, 10S, 11S, 12S, 13S, 14S, 15S, 16S, 17S, 18S, 19S, 20S, 21S, 22S, 23S, 24S, 25S, 26S, 27S, 28S, 29S, 30S, 31S, 32S, 33S, 34S, 35S, 36S, 37S, 39S, 40S, 41S, 42S, 43S, 44S, 45S, 46S, 47S, 48S, 49S, 50S, 51S, 52S, 53S, 54S, 55S, 56S, 57S, 58S, 1AS, 2AS, 3AS, 4AS, or 5AS.

### H. Formulations

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof. In some embodiments, the composition is formulated for ocular delivery.

### II. METHODS AND USES

Disclosed herein, in some embodiments, are methods of administering a composition described herein to a subject. Some embodiments relate to use a composition described herein, such as administering the composition to a subject.

Some embodiments relate to a method of treating a disorder in a subject in need thereof. Some embodiments relate to use of a composition described herein in the method of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subject with the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

### A. Disorders

Some embodiments of the methods described herein include treating a disorder in a subject in need thereof. Some embodiments include administering a composition described herein to a subject having the disorder. In some embodiments, the disorder is a chronic kidney disease, diabetic nephropathy, gout, hyperuricemia, hypertension, cerebrovascular disease, type 2 diabetes, metabolic syndrome, obesity, hyperlipidemia, hypertriglyceridemia, glaucoma, ocular hypertension, retinal diseases, age-related macular degeneration, choroidal neovascularization, geographic atrophy, diabetic retinopathy, non-alcoholic fatty liver disease, fibrotic liver disease, liver fibrosis, cirrhosis, or hair loss disorder. In some embodiments, the disorder comprises a kidney disease. In some embodiments, the disorder comprises chronic kidney disease. In some embodiments, the disorder comprises diabetic nephropathy. In some embodiments, the disorder comprises gout. In some embodiments, the disorder comprises hyperuricemia. In some embodiments, the disorder comprises hypertension. In some embodiments, the disorder comprises cerebrovascular disease. In some embodiments, the disorder comprises a metabolic disorder. In some embodiments, the disorder comprises diabetes. In some embodiments, the disorder comprises type 2 diabetes. In some embodiments, the disorder comprises metabolic syndrome. In some embodiments, the disorder comprises obesity. In some embodiments, the disorder comprises hyperlipidemia. In some embodiments, the disorder comprises hypertriglyceridemia. In some embodiments, the disorder comprises glaucoma. In some embodiments, the disorder comprises ocular hypertension. In some embodiments, the disorder comprises retinal diseases. In some embodiments, the disorder comprises age-related macular degeneration. In some embodiments, the disorder comprises choroidal neovascularization. In some embodiments, the disorder comprises geographic atrophy. In some embodiments, the disorder comprises diabetic retinopathy. In some embodiments, the disorder comprises a liver disease. In some embodiments, the disorder comprises non-alcoholic fatty liver disease. In some embodiments, the disorder comprises fibrotic liver disease. In some embodiments, the disorder comprises liver fibrosis. In some embodiments, the disorder comprises cirrhosis. In some embodiments, the disorder comprises hair loss.

### B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat. In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human. In some embodiments, the subject is male. In some embodiments, the subject is female.

In some embodiments, the subject has a body mass index (BMI) of 25, 26, 27, 28, 29, 30, 31, 32, 33, 34, 35, 36, 37, 38, 39, 40, 41, 42, 43, 44, 45, 46, 47, 48, 49, 50, or more, or a range defined by any two of the aforementioned integers. In some embodiments, the subject is overweight. In some embodiments, the subject has a BMI of 25 or more. In some embodiments, the subject has a BMI of 25-29. In some embodiments, the subject is obese. In some embodiments, the subject has a BMI of 30 or more. In some embodiments, the subject has a BMI of 30-39. In some embodiments, the subject has a BMI of 40-50. In some embodiments, the subject has a BMI of 25-50.

In some embodiments, the subject is ≥90 years of age. In some embodiments, the subject is ≥85 years of age. In some embodiments, the subject is ≥80 years of age. In some embodiments, the subject is ≥70 years of age. In some embodiments, the subject is ≥60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is ≥0 years of age.

In some embodiments, the subject is ≤100 years of age. In some embodiments, the subject is ≤90 years of age. In some embodiments, the subject is ≤85 years of age. In some embodiments, the subject is ≤80 years of age. In some embodiments, the subject is ≤70 years of age. In some embodiments, the subject is ≤60 years of age. In some embodiments, the subject is ≤50 years of age. In some embodiments, the subject is ≤40 years of age. In some embodiments, the subject is ≤30 years of age. In some embodiments, the subject is ≤20 years of age. In some embodiments, the subject is ≤10 years of age. In some embodiments, the subject is ≤1 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

### C. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline glomerular filtration rate (GFR) or estimated glomerular filtration rate (eGFR) measurement, a baseline creatinine measurement, a baseline blood urea nitrogen (BUN) measurement, a baseline proteinuria measurement, a baseline microalbuminuria measurement, a baseline blood urate measurement, a baseline urine albumin creatine ratio, a baseline systolic blood pressure (SBP) measurement, a baseline diastolic blood pressure (DBP) measurement, a baseline mean arterial pressure measurement, a baseline pulse pressure measurement, a baseline intraocular pressure (IOP) measurement, a baseline cup-disc ratio, a baseline RNFL thickness measurement, a baseline optic nerve head cupping measurement, a baseline RPE pigmentation and reflectivity measurement, a baseline retinal thickness measurement, a baseline drusen measurement, a baseline macular hemorrhage measurement, a baseline choroidal neovascularization measurement, a baseline edema measurement, a baseline microaneurysm measurement, a baseline intraretinal hemorrhage measurement, a baseline macular ischemia measurement, a baseline neovascularization measurement, a baseline vitreous hemorrhage measurement, a baseline traction retinal detachment measurement, a baseline hemoglobin A1C measurement, a baseline body mass index (BMI), a baseline body weight measurement, a baseline waist circumference measurement, a baseline hip circumference measurement, a baseline waist-hip ratio (WHR), a baseline body fat percentage, a baseline blood glucose measurement, a baseline glucose tolerance measurement, a baseline insulin sensitivity measurement, a baseline blood triglyceride measurement, a baseline non-HDL cholesterol measurement, a baseline alanine aminotransferase (ALT) measurement, a baseline aspartate aminotransferase (AST) measurement, a baseline liver fat percentage (LFP) measurement, a baseline liver fibrosis measurement, a baseline liver fibrosis score, a baseline NAFLD activity score, a baseline blood gamma-glutamyl transferase (GGT) measurement, a baseline hair count measurement, a baseline hair thickness measurement, a baseline hair density measurement, a baseline SOS2 protein measurement, or a baseline SOS2 mRNA measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device. In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, a fluorescence assay, or a chromatography (e.g. HPLC) assay. In some embodiments, the baseline measurement is obtained by PCR.

In some embodiments, the baseline measurement is a baseline GFR or eGFR measurement. In some embodiments, the baseline measurement is a baseline GFR measurement. In some embodiments, the baseline measurement is a baseline eGFR measurement. The baseline GFR or eGFR measurement may be indicated in units of volume per time (e.g. mL/min). The baseline GFR measurement may be obtained using a baseline clearance measurement such as a baseline creatinine clearance measurement. The baseline GFR may also be determined by injecting insulin, sinistrin, a radioactive tracer, or cystatin C, and determining a baseline clearance rate. The baseline eGFR measurement may be also be obtained using a clearance estimate such as an estimation of serum creatinine clearance. The baseline GFR or eGFR may be 100-130 mL/min/1.73 m², 90-100 mL/min/1.73 m². The baseline GFR or eGFR may be below 90 or 100 mL/min/1.73 m². The baseline GFR or eGFR may be indicative of normal kidney function, CKD1, CKD2, CKD3, CKD4, or CKD5, as indicated by the following kidney function index:
Normal kidney function—GFR above 90 mL/min/1.73 m² (optionally with no proteinuria)
CKD1—GFR above 90 mL/min/1.73 m² (optionally with evidence of kidney damage)
CKD2 (mild)—GFR of 60 to 89 mL/min/1.73 m² (optionally with evidence of kidney damage)
CKD3 (moderate)—GFR of 30 to 59 mL/min/1.73 m²
CKD4 (severe)—GFR of 15 to 29 mL/min/1.73 m²
CKD5 kidney failure—GFR less than 15 mL/min/1.73 m²

In some embodiments, the baseline measurement is a baseline creatinine measurement. In some embodiments, the baseline creatinine measurement is a baseline creatinine concentration. In some embodiments, the baseline creatinine measurement is a baseline circulating (e.g. serum or plasma) creatinine measurement. In some embodiments, the baseline creatinine measurement is a baseline urine creatinine measurement. In some embodiments, the baseline creatinine measurement is obtained by an assay such as an immunoassay, a colorimetric assay, or a fluorescence assay. The baseline circulating creatinine measurement may be about 0.5-1.3 mg/dL. The baseline creatinine may be measured in a fluid sample. The baseline circulating creatinine measurement may be above 1.3 mg/dL. The baseline circulating creatinine measurement may be within, above, or below a reference range. The baseline urine creatinine measurement may be within, above, or below a reference range. Typical human reference ranges for serum creatinine are 0.5 mg/dL to 1.0 mg/dL for women or 0.7 mg/dL to 1.2 mg/dL for men. The significance of a single creatinine value may be interpreted in light of the patient's muscle mass. A patient with a greater muscle mass may have a higher creatinine concentration. While a baseline serum creatinine of 2.0 mg/dL (177 mol/L) may indicate normal kidney function in a male body builder, a serum creatinine of 1.6 mg/dL (110 mol/L) may indicate significant renal disease in an elderly female. Males may typically produce approximately 150 mol to 200 mol of creatinine per kilogram of body weight per 24 h while females may produce approximately 100 mol/kg/24 h to 150 mol/kg/24 h. In normal circumstances, all this daily creatinine production is excreted in the urine, which may be included in a baseline urine creatinine measurement.

In some embodiments, the baseline measurement is a baseline blood urea nitrogen (BUN) measurement. In some embodiments, the baseline BUN measurement is a baseline BUN concentration. In some embodiments, the baseline BUN measurement is a baseline circulating BUN measurement. In some embodiments, the baseline BUN measurement is obtained by an assay such as an immunoassay, a colorimetric assay, or a fluorescence assay. In some embodiments, the baseline BUN is 6-20 mg/dL. In some embodiments, the baseline BUN is over 20 mg/dL. A normal BUN range is 6-20 mg/dL. In some embodiments, the baseline measurement is a baseline BUN/creatinine ratio.

In some embodiments, the baseline measurement is a baseline proteinuria measurement. “Proteinuria” may describe an increase (e.g. a moderate increase) in a level of urine protein. The baseline proteinuria measurement may be indicated as a concentration, a ratio, or a mass/unit time (e.g. mg/mmol urine, protein/creatinine, or mg protein/hr). In some embodiments, the baseline proteinuria measurement includes a baseline proteinuria concentration. In some embodiments, the baseline proteinuria measurement is a baseline urine protein measurement. In some embodiments, the baseline proteinuria measurement is obtained by an assay such as an immunoassay, a colorimetric assay, or a fluorescence assay. In some embodiments, the baseline proteinuria measurement is indicative of proteinuria in the subject. Proteinuria can be diagnosed from a 24-hour urine collection or, from an elevated concentration in a spot sample. In some embodiments, the baseline measurement is a baseline urine protein/creatinine ratio.

In some embodiments, the baseline measurement is a baseline microalbuminuria measurement. “Microalbuminuria” may describe an increase (e.g. a moderate increase) in a level of urine albumin. The baseline microalbuminuria measurement may be indicated as a concentration, a ratio, or a mass/unit time (e.g. mg/mmol urine, albumin/creatinine, or mg albumin/hr). In some embodiments, the baseline microalbuminuria measurement includes a baseline microalbuminuria concentration. In some embodiments, the baseline microalbuminuria measurement is a baseline urine microalbuminuria measurement. In some embodiments, the baseline microalbuminuria measurement is obtained by an assay such as an immunoassay, a colorimetric assay, or a fluorescence assay. In some embodiments, the baseline microalbuminuria measurement is indicative of microalbuminuria in the subject. Microalbuminuria can be diagnosed from a 24-hour urine collection (between 30-300 mg/24 hours) or, from an elevated concentration in a spot sample (20 to 200 mg/l). In some embodiments, the baseline measurement is a baseline urine albumin/creatinine ratio. The baseline microalbuminuria measurement may include a microalbuminuria measurement within a range or amount defined in Table 2.

TABLE 2
Microalbuminuria reference values

| | | Lower | Upper | |
|---|---|---|---|---|
| | Individual | limit | limit | Unit |
| 24 h urine | | 30 | 300 | mg/24 h (milligram |
| collection | | | | albumin per 24 hours) |
| Short-time | | 20 | 200 | μg/min (microgram |
| urine collection | | | | albumin per minute) |
| Spot urine | | 30 | 300 | mg/L (milligram albumin |
| albumin sample | | | | per liter of urine) |
| Spot urine | Women | 3.5 | 25 or | mg/mmol (milligram |
| albumin/ | | | 35 | albumin per millimole |
| creatinine ratio | | | | creatinine) |
| | | 30 | 400 | μg/mg (microgram |
| | | | | albumin per milligram |
| | | | | creatinine) |
| | Men | 2.5 or | 25 or | mg/mmol |
| | | 3.5 | 35 | |
| | | 30 | 300 | μg/mg |

In some embodiments, the baseline measurement is a baseline blood urate measurement. In some embodiments, the baseline blood urate measurement is a baseline blood urate concentration. In some embodiments, the baseline blood urate measurement is obtained by an assay such as an immunoassay, a colorimetric assay, or a fluorescence assay. In some embodiments, the baseline blood urate measurement is indicative of hyperuricemia. Serum uric acid concentrations greater than 6 mg/dL (e.g. for females), greater than 7 mg/dL (e.g. for men), or greater than 5.5 mg/dL (e.g. for a subject under 18 years old) may be indicative of hyperuricemia.

Some embodiments of the methods described herein include obtaining the baseline measurement of the subject by measuring blood pressure (e.g. systolic or diastolic) with a sphygmomanometer in which a healthcare professional places a cuff around an arm of the subject and inflates the cuff with a pump until the circulation is cut off A small valve slowly deflates the cuff, and the healthcare professional measures the pressure with the aid of a stethoscope that is placed over the arm of the subject in order to listen for the sound of the blood pulsing through the arteries. The first measurement in which blood rushes is the systolic blood pressure (SBP), and after the sound fades, the second number indicates the diastolic blood pressure (DBP), which is a measure the blood pressure of the heart at rest. The mean arterial pressure (MAP) is an average blood pressure of the subject during a single cardiac cycle. The MAP can be measured directly using methods such as applanation tonometry or it can be approximated by using a formula in which the diastolic blood pressure is doubled and added to the systolic blood pressure and that composite sum is then divided by 3 to estimate MAP. The pulse pressure can be calculated by subtracting the systolic pressure from the diastolic pressure.

In some embodiments, the baseline measurement is a baseline systolic blood (SBP) pressure measurement. In some embodiments, the baseline SBP measurement is measured in mm of mercury (mm Hg). In some embodiments, the SBP measurement is obtained with a sphygmomanometer. The baseline SBP measurement may be indicative of normal blood pressure. For most adults, normal SBP at rest is within the range of 100-130 mmHg. For most adults, hypertension is present if the resting blood pressure is persistently at or above 130/80 or 140/90 mmHg. The baseline SBP measurement may be indicative of hypertension (e.g. at least 130 mmHg, or at least 140 mmHg). The baseline SBP measurement may include a baseline cerebral SBP measurement.

In some embodiments, the baseline measurement is a baseline diastolic blood (DBP) pressure measurement. In some embodiments, the baseline DBP measurement is measured in mm Hg. In some embodiments, the DBP measurement is obtained with a sphygmomanometer. The baseline DBP measurement may be indicative of normal blood pressure. For most adults, normal DBP at rest is within the range of 60-80 mmHg. The baseline DBP measurement may be indicative of hypertension (e.g. at least 80 mmHg, or at least 90 mmHg). The baseline DBP measurement may include a baseline cerebral DBP measurement.

In some embodiments, the baseline measurement is a baseline mean arterial pressure (MAP). In some embodiments, the baseline MAP is measured in mm of mercury (mm Hg). In some embodiments, the MAP measurement is obtained with a sphygmomanometer. The baseline MAP may be indicative of normal blood pressure. For most adults, MAP at rest is in a range of 70-100 mmHg. The baseline MAP measurement may be indicative of hypertension (e.g. greater than 100 mmHg).

In some embodiments, the baseline measurement is a baseline pulse pressure. In some embodiments, the baseline pulse pressure is measured in mm of mercury (mm Hg). In some embodiments, the pulse pressure measurement is obtained with a sphygmomanometer. The baseline pulse pressure may be indicative of normal blood pressure. For most adults, normal pulse pressure at rest is less than 40 mm Hg. The baseline pulse pressure may be indicative of hypertension (e.g. at least 50 mm Hg or at least 60 mm Hg).

In some embodiments, the baseline measurement is a baseline intraocular pressure (IOP) measurement. The baseline IOP may be measured using a tonometer. The baseline IOP measurement may be in millimeters of mercury (mmHg). The baseline IOP measurement may be indicative of a normal IOP. The baseline IOP measurement may be indicative of abnormal or high IOP. A normal IPO measurement may be between 10 mmHg and 20 mmHg. The baseline IOP measurement may be above 20 mmHg.

In some embodiments, the baseline measurement is a baseline measurement of optic nerve head cupping. The baseline measurement of optic nerve head cupping may be a cup-disc ratio measurement. The baseline cup-disc ratio may be measured using a slit lamp. The baseline cup-disc ratio measurement may be indicative of a normal cup-disc ratio. The baseline cup-disc ratio measurement may be indicative of a high or abnormal cup-disc ratio. A normal cup-disc ratio measurement may be between 0.4 and 0.8. The baseline cup-disc ratio measurement may be above 0.8.

In some embodiments, the baseline measurement is a baseline retinal nerve fiber layer (RNFL) thickness measurement. The baseline RNFL thickness may be measured using optical coherence tomography. The baseline RNFL thickness measurement may be in μm. The baseline RNFL thickness measurement may be indicative of a normal RNFL thickness measurement. The baseline RNFL thickness measurement may be indicative of a low or abnormal RNFL thickness measurement. A normal RNFL thickness measurement may be around 100 μm. The baseline RNFL thickness measurement may be below 90 μm.

In some embodiments, the baseline measurement is a baseline retinal thickness measurement. The baseline retinal thickness may be measured using optical coherence tomography. The baseline retinal thickness measurement may be in μm. The baseline retinal thickness measurement may be indicative of a normal retinal thickness measurement. The baseline retinal thickness measurement may be indicative of a high or abnormal retinal thickness measurement. A normal retinal thickness measurement may be about 190 μm to 250 m. The baseline retinal thickness measurement may be above 250 μm.

In some embodiments, the baseline measurement is a baseline edema measurement. The baseline edema may be measured using optical coherence tomography. The baseline edema measurement may be in μm. The baseline edema measurement may be indicative of a normal edema measurement. The baseline edema measurement may be indicative of a high or abnormal edema measurement. A normal edema measurement may be between 190 μm and 250 μm. The baseline edema measurement may be above 250 μm.

In some embodiments, the baseline measurement is a baseline RPE pigmentation and reflectivity measurement. The baseline RPE pigmentation and reflectivity measurement may be measured using optical coherence tomography. The baseline RPE pigmentation and reflectivity measurement may be indicative of a normal RPE pigmentation and reflectivity measurement. The baseline RPE pigmentation and reflectivity measurement may be indicative of abnormal RPE pigmentation and reflectivity.

In some embodiments, the baseline measurement is a baseline drusen measurement. The baseline drusen may be measured using an eye exam or retinal photography. The baseline drusen measurement may be the size of the drusen or the number of the drusen. The baseline drusen measurement may be indicative of a normal drusen measurement. The baseline drusen measurement may be indicative of a high or abnormal drusen measurement.

In some embodiments, the baseline measurement is a baseline hemorrhage measurement. The baseline hemorrhage measurement may be a vitreous hemorrhage. The baseline hemorrhage measurement may be an intraretinal hemorrhage. The baseline hemorrhage may be a macular hemorrhage. The baseline hemorrhage may be measured using retinal photography. The baseline hemorrhage measurement may be indicative of a normal hemorrhage measurement. The baseline hemorrhage measurement may be indicative of an abnormal or high hemorrhage measurement.

In some embodiments, the baseline measurement is a baseline macular ischemia measurement. The baseline macular ischemia may be measured using fluorescein angiography or optical coherence tomography angiography. The baseline macular ischemia measurement may be a measurement of the foveal avascular zone. The baseline macular ischemia measurement may be indicative of a normal macular ischemia measurement. The baseline macular ischemia measurement may be indicative of a high or abnormal macular ischemia measurement. A normal foveal avascular zone measurement may have a diameter between 0.5 mm to 0.6 mm. The baseline foveal avascular zone measurement may have a diameter above 0.6 mm.

In some embodiments, the baseline measurement is a baseline microaneurysm measurement. The baseline microaneurysm may be measured using fluorescein angiography. The baseline microaneurysm measurement may be the count of microaneurysms. The baseline microaneurysm measurement may be indicative of a normal microaneurysm measurement. The baseline microaneurysm measurement may be indicative of high or abnormal microaneurysm measurement.

In some embodiments, the baseline measurement is a baseline neovascularization measurement. The baseline neovascularization measurement may be a choroidal neovascularization measurement. The baseline neovascularization measurement may be measured using imaging techniques such as fluorescein angiography. The baseline neovascularization measurement may be an area of the neovascularization. The baseline neovascularization measurement may be indicative of a normal neovascularization measurement. The baseline neovascularization measurement may be indicative of a high or abnormal neovascularization measurement.

In some embodiments, the baseline measurement is a baseline traction retinal detachment measurement. The baseline traction retinal detachment may be measured using imaging techniques, including optical coherence tomography. The baseline traction retinal detachment measurement may be indicative of a normal retina. The baseline traction retinal detachment measurement may be indicative of macular degeneration or diabetic retinopathy.

In some embodiments, the baseline measurement is a baseline hemoglobin A1C measurement. In some embodiments, the baseline hemoglobin A1C measurement is a baseline hemoglobin A1C concentration. In some embodiments, the baseline hemoglobin A1C measurement is a baseline circulating hemoglobin A1C measurement. In some embodiments, the baseline hemoglobin A1C measurement is obtained by an assay such as an immunoassay, a colorimetric assay, a fluorescence assay, or HPLC. The baseline hemoglobin A1C measurement may be indicative of a healthy normal A1C measurement. The healthy normal hemoglobin A1C measurement may be below 48 mmol/mol (6.5 DCCT %). The healthy normal hemoglobin A1C measurement may be below 53 mmol/mol (7.0 DCCT %). The baseline hemoglobin A1C measurement may be indicative of diabetes of pre-diabetes. A baseline hemoglobin A1C measurement above 48 mmol/mol, or above 53 mmol/mol may indicate diabetes of pre-diabetes. The baseline hemoglobin A1C measurement may be indicative of diabetes. The baseline hemoglobin A1C measurement may be indicative of pre-diabetes. In some cases, the baseline hemoglobin A1C measurement is below 5.7 DCCT % (e.g. indicative of a normal healthy diagnosis). In some cases, the baseline hemoglobin A1C measurement is between 5.7 and 6.4 DCCT % (e.g. indicative of prediabetes). In some cases, the baseline hemoglobin A1C measurement is above 6.4 DCCT % (e.g. indicative of diabetes).

In some embodiments, the baseline measurement is a baseline body mass measurement. In some embodiments, the baseline body mass measurement is a baseline body mass index (BMI). BMI may be defined as a body mass divided by the square of body height, and may be expressed in units of kg/m². Body mass (body weight) may be obtained using a scale. Body height may be measured using a ruler or a measuring tape. Body height may include the height of a standing subject. Body height may include a distance from the bottom of a subject's feet to the top of the subject's head. BMI may include BMI prime. The subject may have a baseline BMI in a range exemplified in Table 3.

TABLE 3
BMI Examples

| | BMI | | BMI | |
|---|---|---|---|---|
| | (kg/m²) | | Prime | |
| Category | from | to | from | to |
| Very severely underweight | | 15 | | 0.6 |
| Severely underweight | 15 | 16 | 0.6 | 0.64 |
| Underweight | 16 | 18.5 | 0.64 | 0.74 |
| Normal (healthy weight) | 18.5 | 25 | 0.74 | 1 |
| Overweight | 25 | 30 | 1 | 1.2 |
| Obese Class I (Moderately obese) | 30 | 35 | 1.2 | 1.4 |
| Obese Class II (Severely obese) | 35 | 40 | 1.4 | 1.6 |
| Obese Class III (Very severely obese) | 40 | | 1.6 | |

In some embodiments, the baseline measurement is a baseline waist circumference measurement. A baseline waist circumference measurement may be obtained using a measuring tape.

In some embodiments, the baseline measurement is a baseline hip circumference measurement. A baseline hip circumference measurement may be obtained using a measuring tape.

In some embodiments, the baseline measurement is a baseline waist-hip ratio. A baseline waist-hip ratio may be obtained using a measuring tape.

In some embodiments, the baseline measurement is a baseline body fat percentage. A baseline body fat percentage may be obtained using underwater weighing, whole-body air displacement plethysmography, near-infrared interactance, dual energy X-ray absorptiometry, bioelectrical impedance, or a skinfold test.

In some embodiments, the baseline measurement is a baseline glucose measurement. In some embodiments, the baseline glucose measurement is a baseline glucose concentration (for example, mg/dL). In some embodiments, the baseline glucose measurement comprises a baseline glucose concentration. In some embodiments, the baseline glucose measurement is a baseline circulating glucose measurement. In some embodiments, the baseline glucose measurement is obtained by an assay such as an immunoassay, a colorimetric assay, or a fluorescence assay. In some embodiments, the baseline glucose measurement is obtained

In some embodiments, the baseline glucose measurement comprises a baseline glucose tolerance test. In some embodiments, the baseline glucose tolerance test comprises administering glucose to the subject, and then obtaining multiple baseline glucose measurements over time after administering the glucose to the subject. In some embodiments, the glucose is administered orally. In some embodiments, the glucose is administered by injection. In some embodiments, the multiple baseline glucose measurements are integrated into a baseline glucose area under the curve (AUC) measurement. In some embodiments, the baseline glucose tolerance test is performed on the subject in a fasted state such as after an overnight fast. In some embodiments, the baseline glucose measurement comprises a baseline glucose measurement other than a baseline glucose tolerance test.

In some embodiments, the baseline measurement is a baseline insulin measurement. In some embodiments, the baseline insulin measurement is a baseline insulin sensitivity measurement. In some embodiments, the baseline insulin sensitivity measurement is obtained using a glucose clamp technique such as a hyperinsulinemic euglycemic clamp. In some embodiments, the baseline insulin measurement is a baseline insulin concentration. In some embodiments, the baseline insulin measurement comprises a baseline insulin concentration. In some embodiments, the baseline insulin measurement is a baseline circulating insulin measurement. In some embodiments, the baseline insulin measurement is obtained by an assay such as an immunoassay (for example, an ELISA or an immunoblot), a colorimetric assay, or a fluorescence assay. In some embodiments, the baseline insulin sensitivity measurement comprises a baseline glucose tolerance test. In some embodiments, the baseline insulin sensitivity measurement comprises a baseline insulin sensitivity measurement other than a baseline glucose tolerance test.

In some embodiments, the baseline insulin measurement comprises a baseline insulin response test. In some embodiments, the baseline insulin response test comprises administering glucose to the subject and then obtaining multiple baseline insulin measurements over time after administering the glucose to the subject. In some embodiments, the glucose is administered orally. In some embodiments, the glucose is administered by injection. In some embodiments, the multiple baseline insulin measurements are integrated into a baseline insulin AUC measurement. In some embodiments, the baseline insulin response test is performed on the subject in a fasted state such as after an overnight fast.

In some embodiments, the baseline insulin measurement comprises a baseline glucose response test. In some embodiments, the baseline glucose response test comprises administering insulin to the subject, and then obtaining multiple baseline glucose measurements over time after administering the insulin to the subject. In some embodiments, the insulin is administered by injection. In some embodiments, the multiple baseline glucose measurements are integrated into a baseline glucose AUC measurement. In some embodiments, the multiple baseline glucose measurements are obtained with a glucometer. In some embodiments, the glucose response test is performed on the subject in a fasted state such as after an overnight fast. In some embodiments, the glucose response test is performed on the subject after administering food, drink, or glucose to the subject.

In some embodiments, the baseline measurement is a baseline triglyceride measurement. In some embodiments, the baseline triglyceride measurement is a baseline triglyceride concentration (for example, mg/dL). In some embodiments, the baseline triglyceride measurement is a baseline circulating triglyceride measurement. In some embodiments, the baseline triglyceride measurement a baseline circulating triglyceride measurement above 150 mg/dL. In some embodiments, the baseline triglyceride measurement is obtained by an assay such as an immunoassay, a colorimetric assay, or a fluorescence assay.

In some embodiments, the baseline measurement is a baseline cholesterol measurement. In some embodiments, the baseline cholesterol concentration is a baseline total cholesterol measurement. In some embodiments, the baseline cholesterol concentration is a baseline non-high density lipoprotein (HDL) cholesterol measurement. In some embodiments, the baseline cholesterol concentration is a baseline low density lipoprotein (LDL) cholesterol measurement. In some embodiments, the baseline cholesterol measurement is a baseline cholesterol concentration. In some embodiments, the baseline cholesterol measurement is a baseline circulating cholesterol measurement. In some embodiments, the baseline cholesterol measurement is a baseline blood cholesterol measurement. In some embodiments, the baseline cholesterol measurement is obtained by an assay such as an immunoassay, a colorimetric assay, or a fluorescence assay.

In some embodiments, the baseline measurement is a baseline liver enzyme measurement. In some embodiments, the baseline liver enzyme measurement is a baseline alanine aminotransferase (ALT) measurement. In some embodiments, the baseline liver enzyme measurement is a baseline aspartate aminotransferase (AST) measurement. In some embodiments, the baseline liver enzyme measurement comprises an ALT/AST ratio, or comprises an AST/ALT ratio. In some embodiments, the baseline liver enzyme measurement is obtained by an assay such as an immunoassay, a colorimetric assay, a fluorescence assay, or HPLC.

In some embodiments, the baseline measurement is a baseline alanine aminotransferase (ALT) measurement. In some embodiments, the baseline ALT measurement is a baseline ALT concentration (for example, Units/dL). In some embodiments, the baseline ALT measurement is a baseline circulating ALT measurement, for example, a baseline blood, serum, or plasma ALT measurement. In some embodiments, the baseline ALT measurement is obtained by an assay such as an immunoassay, a colorimetric assay, or a fluorescence assay. In some embodiments, the baseline ALT measurement is within a reference range of 34 IU/L or lower (e.g. for a female subject) or within a reference range of 45 IU/L or lower (e.g. for a male subject). In some embodiments, the baseline ALT measurement is above the reference range.

In some embodiments, the baseline measurement is a baseline aspartate aminotransferase (AST) measurement. In some embodiments, the baseline AST measurement is a baseline AST concentration (for example, Units/L). In some embodiments, the baseline AST measurement is a baseline circulating AST measurement, for example, a baseline blood, serum, or plasma AST measurement. In some embodiments, the baseline AST measurement is obtained by an assay such as an immunoassay, a colorimetric assay, or a fluorescence assay. In some embodiments, the baseline AST measurement is within a reference range of 6-34 IU/L (e.g. for a female subject) or within a reference range of 8-40 IU/L (e.g. for a male subject). In some embodiments, the baseline AST measurement is above the reference range. In some embodiments, the baseline AST measurement is below the reference range.

In some embodiments, the baseline measurement is a baseline liver steatosis measurement. In some embodiments, the baseline liver steatosis measurement is a baseline liver fat percentage (LFP) measurement. In some embodiments, the baseline measurement is a baseline LFP measurement. In some embodiments, the baseline LFP measurement is indicated as a mass/mass percentage of fat/total tissue. In some embodiments, the baseline LFP measurement is indicated as a mass/volume percentage of fat/total tissue. In some embodiments, the baseline LFP measurement is indicated as a volume/mass percentage of fat/total tissue. In some embodiments, the baseline LFP measurement is indicated as a volume/volume percentage of fat/total tissue. In some embodiments, the baseline LFP measurement is indicated as a score. In some embodiments, the baseline LFP measurement is obtained noninvasively. In some embodiments, the baseline LFP measurement is obtained by a medical imaging device. In some embodiments, the baseline LFP measurement is obtained by a device such as a medical resonance imaging (MRI) device, a magnetic resonance spectroscopy device, a computed tomography device, a controlled attenuation parameter (CAP), a transient elastography device, or an ultrasound device. In some embodiments, the baseline LFP measurement is obtained in a liver sample. In some embodiments, the baseline LFP measurement comprises a baseline liver triglyceride measurement. In some embodiments, the baseline LFP measurement is obtained by an assay such as an immunoassay, a colorimetric assay, or a fluorescence assay. In some embodiments, the baseline LFP measurement or the baseline LFP measurement is obtained using a scoring system upon a visual inspection of a sample such as a histological sample. In some embodiments, the baseline LFP measurement or the baseline LFP measurement is obtained using a stain with an affinity to fats, such as a lysochrome diazo dye.

In some embodiments, the baseline measurement is a baseline liver fibrosis measurement. In some embodiments, the baseline liver fibrosis measurement is a baseline liver fibrosis score (LFS). In some embodiments, the LFS comprises a score of 0, 1, 2, 3, or 4, or a range of scores defined by any two of the aforementioned numbers. In some embodiments, the LFS comprises a score of 0-4. In some embodiments, the LFS is obtained using a scoring system exemplified in Table 4. In some embodiments, the baseline LFS measurement is obtained noninvasively. In some embodiments, the baseline LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the baseline LFS measurement is obtained in a liver sample. In some embodiments, the baseline LFS is obtained by an assay such as an immunoassay, a colorimetric assay, or a fluorescence assay. In some embodiments, the LFS is obtained using one or more indirect markers or measures of liver fibrosis such as an aspartate aminotransferase-to-platelet ratio index (APRI), a Fibrosis-4 (FIB-4) index, a FibroIndex, a Forns Index, a Hepascore, or a FibroTest. In some embodiments, the LFS is obtained using one or more indirect markers or measures of liver fibrosis such as a FIBROSpect test or a FIBROSpect II test. In some embodiments, the baseline LFS is obtained by RT-qPCR or RNA sequencing of one or more fibrosis-related genes such as a collagen gene. In some embodiments, the baseline LFS or the baseline LFS is obtained using a scoring system upon a visual inspection of a sample such as a histological sample. In some embodiments, the baseline LFS or the baseline LFS is obtained using a stain with an affinity to collagen.

TABLE 4
Non-Limiting Examples of Liver Fibrosis Scoring Systems

| Score | IASL | Batts-Ludwig | Metavir |
|---|---|---|---|
| 0 | No fibrosis | No fibrosis | No fibrosis |
| 1 | Mild fibrosis | Fibrous portal | Periportal fibrotic |
| | | expansion | expansion |
| 2 | Moderate | Rare bridges | Periportal septae |
| | fibrosis | or septae | (>1 septum) |
| 3 | Severe | Numerous | Portal-central |
| | fibrosis | bridges or septae | septae |
| 4 | Cirrhosis | Cirrhosis | Cirrhosis |

In some embodiments, the baseline liver fibrosis measurement is a baseline nonalcoholic fatty liver disease (NAFLD) fibrosis score. A baseline NAFLD fibrosis score may take into account laboratory test values such as platelet count, albumin, and AST/ALT ratio, and patient characteristics such as BMI, and diabetes status. A baseline NAFLD fibrosis score below −1.455 may be indicative of no fibrosis, mild fibrosis, or moderate fibrosis. A baseline NAFLD fibrosis score between −1.455 and 0.675 may be indicative of severe fibrosis. A baseline NAFLD fibrosis score above 0.675 may be indicative of cirrhosis.

In some embodiments, the baseline measurement is a baseline non-alcoholic fatty liver disease (NAFLD) activity score. In some embodiments, the baseline NAFLD activity score comprises a numerical value such as a number of points. In some embodiments, the numerical value is 0, 1, 2, 3, 4, 5, 6, 7, or 8, or a range defined by any two of the aforementioned numerical values. In some embodiments, the numerical value is 0-8. In some embodiments, the baseline NAFLD activity score comprises a steatosis grade such as a baseline liver fat percentage. In some embodiments, a steatosis grade <5% comprises 0 points in the baseline NAFLD activity score. In some embodiments, a steatosis grade of 5-33% comprises 1 point in the baseline NAFLD activity score. In some embodiments, a steatosis grade of 34-66% comprises 2 points in the baseline NAFLD activity score. In some embodiments, a steatosis grade of >66% comprises 3 points in the baseline NAFLD activity score. In some embodiments, the baseline NAFLD activity score comprises a lobular inflammation grade. In some embodiments, the lobular inflammation grade comprises an assessment of inflammatory foci. In some embodiments, a lobular inflammation grade comprising 0 foci comprises 0 points in the baseline NAFLD activity score. In some embodiments, a lobular inflammation grade comprising 1 focus per a field (such as a 20× field or a 200× field) comprises 1 point in the baseline NAFLD activity score. In some embodiments, a lobular inflammation grade comprising 2-4 foci per field comprises 2 points in the baseline NAFLD activity score. In some embodiments, a lobular inflammation grade comprising >4 foci per field comprises 3 points in the baseline NAFLD activity score. In some embodiments, the baseline NAFLD activity score comprises a liver cell injury grade such as an amount of ballooning cells. In some embodiments, a liver cell injury comprising no ballooning cells comprises 0 points in the baseline NAFLD activity score. In some embodiments, a liver cell injury comprising some new balloon cells comprises 1 points in the baseline NAFLD activity score. In some embodiments, a liver cell injury comprising many ballooning cells or prominent ballooning comprises 2 points in the baseline NAFLD activity score. In some embodiments, the baseline NAFLD activity score is obtained invasively, based on histology, and/or in a liver biopsy.

In some embodiments, the baseline measurement is a baseline gamma-glutamyl transferase (GGT) measurement. In some embodiments, the baseline GGT measurement is a baseline GGT concentration. In some embodiments, the baseline GGT measurement is a baseline blood GGT measurement. In some embodiments, the baseline GGT measurement is obtained by an assay such as an immunoassay, a colorimetric assay, a chromatography assay, or a fluorescence assay.

In some embodiments, the baseline measurement is a baseline hair count. In some embodiments, the baseline hair count is a baseline total hair count. The baseline total hair count may include a baseline vellus hair count and a baseline non-vellus hair count. In some embodiments, the baseline hair count is a baseline vellus hair count. In some embodiments, the baseline hair count is a baseline non-vellus hair count. In some embodiments, the baseline hair count is determined in an area of skin. In some embodiments, the baseline hair count is normalized based on the area of skin. In some embodiments, the baseline hair count is assessed using photography. In some embodiments, the baseline hair count is assessed by phototrichogram. In some embodiments, the baseline hair count is assessed by a macrophotography analysis.

In some embodiments, the baseline measurement is a baseline hair thickness measurement. In some embodiments, the baseline hair thickness measurement is determined in an area of skin. In some embodiments, the baseline hair thickness measurement comprises a width of an individual hair. In some embodiments, the baseline hair thickness measurement comprises widths of multiple individual hairs. In some embodiments, the baseline hair thickness measurement comprises an average of the widths of the multiple individual hairs. In some embodiments, the baseline hair thickness measurement comprises a median of the widths of the multiple individual hairs. The baseline hair thickness measurement may include a baseline vellus hair thickness measurement. The baseline hair thickness measurement may include a baseline non-vellus hair thickness measurement. In some embodiments, the baseline hair thickness measurement is assessed using photography. In some embodiments, the baseline hair thickness measurement is assessed by phototrichogram. In some embodiments, the baseline hair thickness measurement is assessed by a macrophotography analysis.

In some embodiments, the baseline measurement is a baseline hair density measurement. In some embodiments, the baseline hair density measurement is determined in an area of skin. In some embodiments, the baseline hair density measurement comprises a number of hair in the area of skin. In some embodiments, the baseline hair density measurement comprises the number of hair in the area of skin divided by the area of skin. The baseline hair density measurement may include a baseline vellus hair density measurement. The baseline hair density measurement may include a baseline non-vellus hair density measurement. In some embodiments, the baseline hair density measurement is assessed using photography. In some embodiments, the baseline hair density measurement is assessed by phototrichogram. In some embodiments, the baseline hair density measurement is assessed by a macrophotography analysis.

In some embodiments, the baseline measurement is a baseline SOS2 protein measurement. In some embodiments, the baseline SOS2 protein measurement comprises a baseline SOS2 protein level. In some embodiments, the baseline SOS2 protein level is indicated as a mass or percentage of SOS2 protein per sample weight. In some embodiments, the baseline SOS2 protein level is indicated as a mass or percentage of SOS2 protein per sample volume. In some embodiments, the baseline SOS2 protein level is indicated as a mass or percentage of SOS2 protein per total protein within the sample. In some embodiments, the baseline SOS2 protein measurement is a baseline tissue SOS2 protein measurement. Examples of baseline tissue SOS2 protein measurements include a baseline liver SOS2 protein measurement, a baseline kidney SOS2 protein measurement, a baseline eye SOS2 protein measurement, or a baseline adipose tissue SOS2 protein measurement. In some embodiments, the baseline SOS2 protein measurement is a baseline circulating SOS2 protein measurement. In some embodiments, the baseline SOS2 protein measurement is obtained by an assay such as an immunoassay, a colorimetric assay, or a fluorescence assay.

In some embodiments, the baseline measurement is a baseline SOS2 mRNA measurement. In some embodiments, the baseline SOS2 mRNA measurement comprises a baseline SOS2 mRNA level. In some embodiments, the baseline SOS2 mRNA level is indicated as a mass or percentage of SOS2 mRNA per sample weight. In some embodiments, the baseline SOS2 mRNA level is indicated as a mass or percentage of SOS2 mRNA per sample volume. In some embodiments, the baseline SOS2 mRNA level is indicated as a mass or percentage of SOS2 mRNA per total mRNA within the sample. In some embodiments, the baseline SOS2 mRNA level is indicated as a mass or percentage of SOS2 mRNA per total nucleic acids within the sample. In some embodiments, the baseline SOS2 mRNA level is indicated relative to another mRNA level, such as an mRNA level of a housekeeping gene, within the sample. In some embodiments, the baseline SOS2 mRNA measurement is a baseline tissue SOS2 mRNA measurement. Examples of baseline tissue SOS2 mRNA measurements include a baseline liver SOS2 mRNA measurement, a baseline kidney SOS2 mRNA measurement, a baseline eye SOS2 mRNA measurement, or a baseline adipose tissue SOS2 mRNA measurement. In some embodiments, the baseline SOS2 mRNA measurement is a baseline circulating SOS2 mRNA measurement. In some embodiments, the baseline SOS2 mRNA measurement is obtained by an assay such as a polymerase chain reaction (PCR) assay. In some embodiments, the PCR comprises quantitative PCR (qPCR). In some embodiments, the PCR comprises reverse transcription of the SOS2 mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample is obtained from the subject in a fasted state. In some embodiments, the sample is obtained from the subject after an overnight fasting period. In some embodiments, the sample is obtained from the subject in a fed state.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. In some embodiments, the sample comprises serum. In some embodiments, the sample is a serum sample.

In some embodiments, the sample comprises a tissue. In some embodiments, the sample is a tissue sample. In some embodiments, the sample comprises liver tissue. In some embodiments, the sample is a liver sample. In some embodiments, the sample comprises adipose tissue. In some embodiments, the sample is an adipose sample. In some embodiments, the tissue sample comprises brown adipose tissue or white adipose tissue. In some embodiments, the sample comprises kidney tissue. In some embodiments, the sample is a kidney sample. In some embodiments, the sample comprises eye tissue. In some embodiments, the sample is an eye sample. In some embodiments, the sample comprises an eye fluid. In some embodiments, the sample comprises a hair or scalp sample. In some examples, the baseline SOS2 mRNA measurement, or the baseline SOS2 protein measurement, may be obtained in a liver, adipose, eye, or kidney sample from the patient. In some embodiments, the sample comprises cardiac tissue such as ventricular or atrial tissue. In some embodiments, the sample comprises a cerebral tissue or fluid. In some embodiments, the sample comprises a neural tissue or neural fluid. In some embodiments, the sample comprises a muscle tissue or fluid. The sample may comprise or consist of hepatocytes. The sample may comprise or consist of podocytes.

### D. Effects

In some embodiments, the composition or administration of the composition affects a measurement such as include a glomerular filtration rate (GFR) or estimated glomerular filtration rate (eGFR) measurement, a creatinine measurement, a blood urea nitrogen (BUN) measurement, a proteinuria measurement, a microalbuminuria measurement, a blood urate measurement, a urine albumin creatine ratio, a systolic blood pressure (SBP) measurement, a diastolic blood pressure (DBP) measurement, a mean arterial pressure measurement, a pulse pressure measurement, a intraocular pressure (IOP) measurement, a cup-disc ratio, a RNFL thickness measurement, a optic nerve head cupping measurement, a RPE pigmentation and reflectivity measurement, a retinal thickness measurement, a drusen measurement, a macular hemorrhage measurement, a choroidal neovascularization measurement, a edema measurement, a microaneurysm measurement, a intraretinal hemorrhage measurement, a macular ischemia measurement, a neovascularization measurement, a vitreous hemorrhage measurement, a traction retinal detachment measurement, a hemoglobin A1C measurement, a body mass index (BMI), a body weight measurement, a waist circumference measurement, a hip circumference measurement, a waist-hip ratio (WHR), a body fat percentage, a blood glucose measurement, a glucose tolerance measurement, a insulin sensitivity measurement, a blood triglyceride measurement, a non-HDL cholesterol measurement, a alanine aminotransferase (ALT) measurement, a aspartate aminotransferase (AST) measurement, a liver fat percentage (LFP) measurement, a liver fibrosis measurement, a liver fibrosis score, a NAFLD activity score, a blood gamma-glutamyl transferase (GGT) measurement, a hair count measurement, a hair thickness measurement, a hair density measurement, a SOS2 protein measurement, or a SOS2 mRNA measurement, relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, or a PCR assay. In some embodiments, the measurement is obtained by an assay such as an immunoassay, a colorimetric assay, or a fluorescence assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the measurement relative to the baseline measurement. In some embodiments, the reduction is measured in a second tissue sample obtained from the subject after administering the composition to the subject. In some embodiments, the reduction is measured directly in the subject after administering the composition to the subject. In some embodiments, the measurement is decreased by about 2.5% or more, about 5% or more, or about 7.5% or more, relative to the baseline measurement. In some embodiments, the measurement is decreased by about 10% or more, relative to the baseline measurement. In some embodiments, the measurement is decreased by about 20% or more, about 30% or more, about 40% or more, about 50% or more, about 60% or more, about 70% or more, about 80% or more, about 90% or more, relative to the baseline measurement. In some embodiments, the measurement is decreased by no more than about 2.5%, no more than about 5%, or no more than about 7.5%, relative to the baseline measurement. In some embodiments, the measurement is decreased by no more than about 10%, relative to the baseline measurement. In some embodiments, the measurement is decreased by no more than about 20%, no more than about 30%, no more than about 40%, no more than about 50%, no more than about 60%, no more than about 70%, no more than about 80%, no more than about 90%, or no more than about 95% relative to the baseline measurement. In some embodiments, the measurement is decreased by 2.5%, 5%, 7.5%, 10%, 20%, 30%, 40%, 50%, 60%, 70%, 80%, 90%, or 95%, or by a range defined by any of the two aforementioned percentages.

In some embodiments, the composition increases the measurement relative to the baseline measurement. In some embodiments, the increase is measured in a second tissue sample obtained from the subject after administering the composition to the subject. In some embodiments, the increase is measured directly in the subject after administering the composition to the subject. In some embodiments, the measurement is increased by about 2.5% or more, about 5% or more, or about 7.5% or more, relative to the baseline measurement. In some embodiments, the measurement is increased by about 10% or more, relative to the baseline measurement. In some embodiments, the measurement is increased by about 20% or more, about 30% or more, about 40% or more, about 50% or more, about 60% or more, about 70% or more, about 80% or more, about 90% or more, relative to the baseline measurement. In some embodiments, the measurement is increased by about 100% or more, increased by about 250% or more, increased by about 500% or more, increased by about 750% or more, or increased by about 1000% or more, relative to the baseline measurement. In some embodiments, the measurement is increased by no more than about 2.5%, no more than about 5%, or no more than about 7.5%, relative to the baseline measurement. In some embodiments, the measurement is increased by no more than about 10%, relative to the baseline measurement. In some embodiments, the measurement is increased by no more than about 20%, no more than about 30%, no more than about 40%, no more than about 50%, no more than about 60%, no more than about 70%, no more than about 80%, no more than about 90%, or no more than about 100% relative to the baseline measurement. In some embodiments, the measurement is increased by no more than about 100%, increased by no more than about 250%, increased by no more than about 500%, increased by no more than about 750%, or increased by no more than about 1000%, relative to the baseline measurement. In some embodiments, the measurement is increased by 2.5%, 5%, 7.5%, 10%, 20%, 30%, 40%, 50%, 60%, 70%, 80%, 90%, 100%, 250%, 500%, 750%, or 1000%, orby a range defined by any of the two aforementioned percentages.

In some embodiments, the measurement is a GFR or eGFR measurement. In some embodiments, the measurement is a GFR measurement. In some embodiments, the measurement is a eGFR measurement. The GFR or eGFR measurement may be indicated in units of volume per time (e.g. mL/min). The GFR measurement may be obtained using a clearance measurement such as a creatinine clearance measurement. The GFR may also be determined by injecting insulin, sinistrin, a radioactive tracer, or cystatin C, and determining a clearance rate. The eGFR measurement may be also be obtained using a clearance estimate such as an estimation of serum creatinine clearance. The GFR or eGFR may be 100-130 mL/min/1.73 m², 90-100 mL/min/1.73 m². The GFR or eGFR may be below 90 or 100 mL/min/1.73 m². The GFR or eGFR may be indicative of normal kidney function, CKD1, CKD2, CKD3, CKD4, or CKD5, as indicated by a kidney function index.

In some embodiments, the composition increases the GFR measurement relative to the baseline GFR or eGFR measurement. In some embodiments, the composition increases the eGFR measurement relative to the baseline GFR or eGFR measurement. In some embodiments, the increase is measured directly in the subject after administering the composition to the subject. In some embodiments, the GFR or eGFR measurement is increased by about 2.5% or more, about 5% or more, or about 7.5% or more, relative to the baseline GFR or eGFR measurement. In some embodiments, the GFR or eGFR measurement is increased by about 10% or more, relative to the baseline GFR or eGFR measurement. In some embodiments, the GFR or eGFR measurement is increased by about 20% or more, about 30% or more, about 40% or more, about 50% or more, about 60% or more, about 70% or more, about 80% or more, about 90% or more, relative to the baseline GFR or eGFR measurement. In some embodiments, the GFR or eGFR measurement is increased by about 100% or more, increased by about 250% or more, increased by about 500% or more, increased by about 750% or more, or increased by about 1000% or more, relative to the baseline GFR or eGFR measurement. In some embodiments, the GFR or eGFR measurement is increased by no more than about 2.5%, no more than about 5%, or no more than about 7.5%, relative to the baseline GFR or eGFR measurement. In some embodiments, the GFR or eGFR measurement is increased by no more than about 10%, relative to the baseline GFR or eGFR measurement. In some embodiments, the GFR or eGFR measurement is increased by no more than about 20%, no more than about 30%, no more than about 40%, no more than about 50%, no more than about 60%, no more than about 70%, no more than about 80%, no more than about 90%, or no more than about 100% relative to the baseline GFR or eGFR measurement. In some embodiments, the GFR or eGFR measurement is increased by no more than about 100%, increased by no more than about 250%, increased by no more than about 500%, increased by no more than about 750%, or increased by no more than about 1000%, relative to the baseline GFR or eGFR measurement. In some embodiments, the GFR or eGFR measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a creatinine measurement. In some embodiments, the creatinine measurement is a creatinine concentration. In some embodiments, the creatinine measurement is a circulating (e.g. serum or plasma) creatinine measurement. In some embodiments, the creatinine measurement is a urine creatinine measurement. In some embodiments, the creatinine measurement is obtained by an assay such as an immunoassay, a colorimetric assay, or a fluorescence assay. The circulating creatinine measurement may be about 0.5-1.3 mg/dL. The circulating creatinine measurement may be above 1.3 mg/dL. The circulating creatinine measurement may be within, above, or below a reference range. The urine creatinine measurement may be within, above, or below a reference range.

In some embodiments, the composition reduces the creatinine measurement relative to the baseline creatinine measurement. In some embodiments, the reduction is measured in a second fluid sample obtained from the subject after administering the composition to the subject. In some embodiments, the reduction is measured directly in the subject after administering the composition to the subject. In some embodiments, the creatinine measurement is decreased by about 2.5% or more, about 5% or more, or about 7.5% or more, relative to the baseline creatinine measurement. In some embodiments, the creatinine measurement is decreased by about 10% or more, relative to the baseline creatinine measurement. In some embodiments, the creatinine measurement is decreased by about 20% or more, about 30% or more, about 40% or more, about 50% or more, about 60% or more, about 70% or more, about 80% or more, about 90% or more, relative to the baseline creatinine measurement. In some embodiments, the creatinine measurement is decreased by no more than about 2.5%, no more than about 5%, or no more than about 7.5%, relative to the baseline creatinine measurement. In some embodiments, the creatinine measurement is decreased by no more than about 10%, relative to the baseline creatinine measurement. In some embodiments, the creatinine measurement is decreased by no more than about 20%, no more than about 30%, no more than about 40%, no more than about 50%, no more than about 60%, no more than about 70%, no more than about 80%, or no more than about 90% relative to the baseline creatinine measurement. In some embodiments, the creatinine measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a blood urea nitrogen (BUN) measurement. In some embodiments, the BUN measurement is a BUN concentration. In some embodiments, the BUN measurement is a circulating BUN measurement. In some embodiments, the BUN measurement is obtained by an assay such as an immunoassay, a colorimetric assay, or a fluorescence assay. In some embodiments, the BUN is 6-20 mg/dL. In some embodiments, the BUN is over 20 mg/dL. A normal BUN range is 6-20 mg/dL. In some embodiments, the measurement is a BUN/creatinine ratio.

In some embodiments, the composition reduces the BUN measurement relative to the baseline BUN measurement. In some embodiments, the reduction is measured in a second blood sample obtained from the subject after administering the composition to the subject. In some embodiments, the reduction is measured directly in the subject after administering the composition to the subject. In some embodiments, the BUN measurement is decreased by about 2.5% or more, about 5% or more, or about 7.5% or more, relative to the baseline BUN measurement. In some embodiments, the BUN measurement is decreased by about 10% or more, relative to the baseline BUN measurement. In some embodiments, the BUN measurement is decreased by about 20% or more, about 30% or more, about 40% or more, about 50% or more, about 60% or more, about 70% or more, about 80% or more, about 90% or more, relative to the baseline BUN measurement. In some embodiments, the BUN measurement is decreased by no more than about 2.5%, no more than about 5%, or no more than about 7.5%, relative to the baseline BUN measurement. In some embodiments, the BUN measurement is decreased by no more than about 10%, relative to the baseline BUN measurement. In some embodiments, the BUN measurement is decreased by no more than about 20%, no more than about 30%, no more than about 40%, no more than about 50%, no more than about 60%, no more than about 70%, no more than about 80%, or no more than about 90% relative to the baseline BUN measurement. In some embodiments, the BUN measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a proteinuria measurement. The proteinuria measurement may be indicated as a concentration, a ratio, or a mass/unit time (e.g. mg/mmol urine, protein/creatinine, or mg protein/hr). In some embodiments, the proteinuria measurement includes a proteinuria concentration. In some embodiments, the proteinuria measurement is a urine proteinuria measurement. In some embodiments, the proteinuria measurement is obtained by an assay such as an immunoassay, a colorimetric assay, or a fluorescence assay. In some embodiments, the proteinuria measurement is indicative of proteinuria in the subject. In some embodiments, the proteinuria measurement is indicative of a lack of proteinuria in the subject. In some embodiments, the measurement is a urine protein/creatinine ratio.

In some embodiments, the composition reduces the proteinuria measurement relative to the baseline proteinuria measurement. In some embodiments, the reduction is measured in a second fluid sample obtained from the subject after administering the composition to the subject. In some embodiments, the reduction is measured directly in the subject after administering the composition to the subject. In some embodiments, the proteinuria measurement is decreased by about 2.5% or more, about 5% or more, or about 7.5% or more, relative to the baseline proteinuria measurement. In some embodiments, the proteinuria measurement is decreased by about 10% or more, relative to the baseline proteinuria measurement. In some embodiments, the proteinuria measurement is decreased by about 20% or more, about 30% or more, about 40% or more, about 50% or more, about 60% or more, about 70% or more, about 80% or more, about 90% or more, relative to the baseline proteinuria measurement. In some embodiments, the proteinuria measurement is decreased by no more than about 2.5%, no more than about 5%, or no more than about 7.5%, relative to the baseline proteinuria measurement. In some embodiments, the proteinuria measurement is decreased by no more than about 10%, relative to the baseline proteinuria measurement. In some embodiments, the proteinuria measurement is decreased by no more than about 20%, no more than about 30%, no more than about 40%, no more than about 50%, no more than about 60%, no more than about 70%, no more than about 80%, or no more than about 90% relative to the baseline proteinuria measurement. In some embodiments, the proteinuria measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a microalbuminuria measurement. The microalbuminuria measurement may be indicated as a concentration, a ratio, or a mass/unit time (e.g. mg/mmol urine, albumin/creatinine, or mg albumin/hr). In some embodiments, the microalbuminuria measurement includes a microalbuminuria concentration. In some embodiments, the microalbuminuria measurement is a urine microalbuminuria measurement. In some embodiments, the microalbuminuria measurement is obtained by an assay such as an immunoassay, a colorimetric assay, or a fluorescence assay. In some embodiments, the microalbuminuria measurement is indicative of microalbuminuria in the subject. In some embodiments, the microalbuminuria measurement is indicative of a lack of microalbuminuria in the subject. In some embodiments, the measurement is a urine albumin/creatinine ratio. The microalbuminuria measurement may include a microalbuminuria measurement within a range or amount defined in Table 2.

In some embodiments, the composition reduces the microalbuminuria measurement relative to the baseline microalbuminuria measurement. In some embodiments, the reduction is measured in a second fluid sample obtained from the subject after administering the composition to the subject. In some embodiments, the reduction is measured directly in the subject after administering the composition to the subject. In some embodiments, the microalbuminuria measurement is decreased by about 2.5% or more, about 5% or more, or about 7.5% or more, relative to the baseline microalbuminuria measurement. In some embodiments, the microalbuminuria measurement is decreased by about 10% or more, relative to the baseline microalbuminuria measurement. In some embodiments, the microalbuminuria measurement is decreased by about 20% or more, about 30% or more, about 40% or more, about 50% or more, about 60% or more, about 70% or more, about 80% or more, about 90% or more, relative to the baseline microalbuminuria measurement. In some embodiments, the microalbuminuria measurement is decreased by no more than about 2.5%, no more than about 5%, or no more than about 7.5%, relative to the baseline microalbuminuria measurement. In some embodiments, the microalbuminuria measurement is decreased by no more than about 10%, relative to the baseline microalbuminuria measurement. In some embodiments, the microalbuminuria measurement is decreased by no more than about 20%, no more than about 30%, no more than about 40%, no more than about 50%, no more than about 60%, no more than about 70%, no more than about 80%, or no more than about 90% relative to the baseline microalbuminuria measurement. In some embodiments, the microalbuminuria measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a blood urate measurement. In some embodiments, the blood urate measurement is a blood urate concentration. In some embodiments, the blood urate measurement is obtained by an assay such as an immunoassay, a colorimetric assay, or a fluorescence assay. In some embodiments, the blood urate measurement is indicative of hyperuicemia. In some embodiments, the blood urate measurement is indicative of a lack of hyperuricemia. For example, the serum uric acid measurement may be 6 mg/dL or less, 7 mg/dL or less, or 5.5 mg/dL or less.

In some embodiments, the composition reduces the blood urate measurement relative to the baseline blood urate measurement. In some embodiments, the reduction is measured in a second blood sample obtained from the subject after administering the composition to the subject. In some embodiments, the reduction is measured directly in the subject after administering the composition to the subject. In some embodiments, the blood urate measurement is decreased by about 2.5% or more, about 5% or more, or about 7.5% or more, relative to the baseline blood urate measurement. In some embodiments, the blood urate measurement is decreased by about 10% or more, relative to the baseline blood urate measurement. In some embodiments, the blood urate measurement is decreased by about 20% or more, about 30% or more, about 40% or more, about 50% or more, about 60% or more, about 70% or more, about 80% or more, about 90% or more, relative to the baseline blood urate measurement. In some embodiments, the blood urate measurement is decreased by no more than about 2.5%, no more than about 5%, or no more than about 7.5%, relative to the baseline blood urate measurement. In some embodiments, the blood urate measurement is decreased by no more than about 10%, relative to the baseline blood urate measurement. In some embodiments, the blood urate measurement is decreased by no more than about 20%, no more than about 30%, no more than about 40%, no more than about 50%, no more than about 60%, no more than about 70%, no more than about 80%, or no more than about 90% relative to the baseline blood urate measurement. In some embodiments, the blood urate measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a systolic blood (SBP) pressure measurement. In some embodiments, the SBP measurement is measured in mm of mercury (mm Hg). In some embodiments, the SBP measurement is obtained with a sphygmomanometer. The SBP measurement may be indicative of hypertension. The SBP measurement may be indicative of normal blood pressure. The SBP measurement may include a cerebral SBP measurement.

In some embodiments, the composition reduces the SBP measurement relative to the baseline SBP measurement. In some embodiments, the reduction is measured directly in the subject after administering the composition to the subject. In some embodiments, the SBP measurement is decreased by about 2.5% or more, about 5% or more, or about 7.5% or more, relative to the baseline SBP measurement. In some embodiments, the SBP measurement is decreased by about 10% or more, relative to the baseline SBP measurement. In some embodiments, the SBP measurement is decreased by about 20% or more, about 30% or more, about 40% or more, about 50% or more, about 60% or more, about 70% or more, about 80% or more, about 90% or more, relative to the baseline SBP measurement. In some embodiments, the SBP measurement is decreased by no more than about 2.5%, no more than about 5%, or no more than about 7.5%, relative to the baseline SBP measurement. In some embodiments, the SBP measurement is decreased by no more than about 10%, relative to the baseline SBP measurement. In some embodiments, the SBP measurement is decreased by no more than about 20%, no more than about 30%, no more than about 40%, no more than about 50%, no more than about 60%, no more than about 70%, no more than about 80%, or no more than about 90% relative to the baseline SBP measurement. In some embodiments, the SBP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a diastolic blood (DBP) pressure measurement. In some embodiments, the DBP measurement is measured in mm of mercury (mm Hg). In some embodiments, the DBP measurement is obtained with a sphygmomanometer. The DBP measurement may be indicative of hypertension. The DBP measurement may be indicative of normal blood pressure. The DBP measurement may include a cerebral DBP measurement.

In some embodiments, the composition reduces the DBP measurement relative to the baseline DBP measurement. In some embodiments, the reduction is measured directly in the subject after administering the composition to the subject. In some embodiments, the DBP measurement is decreased by about 2.5% or more, about 5% or more, or about 7.5% or more, relative to the baseline DBP measurement. In some embodiments, the DBP measurement is decreased by about 10% or more, relative to the baseline DBP measurement. In some embodiments, the DBP measurement is decreased by about 20% or more, about 30% or more, about 40% or more, about 50% or more, about 60% or more, about 70% or more, about 80% or more, about 90% or more, relative to the baseline DBP measurement. In some embodiments, the DBP measurement is decreased by no more than about 2.5%, no more than about 5%, or no more than about 7.5%, relative to the baseline DBP measurement. In some embodiments, the DBP measurement is decreased by no more than about 10%, relative to the baseline DBP measurement. In some embodiments, the DBP measurement is decreased by no more than about 20%, no more than about 30%, no more than about 40%, no more than about 50%, no more than about 60%, no more than about 70%, no more than about 80%, or no more than about 90% relative to the baseline DBP measurement. In some embodiments, the DBP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a mean arterial pressure (MAP) pressure measurement. In some embodiments, the MAP measurement is measured in mm of mercury (mm Hg). In some embodiments, the MAP measurement is obtained with a sphygmomanometer. The MAP measurement may be indicative of hypertension. The MAP measurement may be indicative of normal blood pressure. The MAP measurement may include a cerebral MAP measurement.

In some embodiments, the composition reduces the MAP measurement relative to the baseline MAP measurement. In some embodiments, the reduction is measured directly in the subject after administering the composition to the subject. In some embodiments, the MAP measurement is decreased by about 2.5% or more, about 5% or more, or about 7.5% or more, relative to the baseline MAP measurement. In some embodiments, the MAP measurement is decreased by about 10% or more, relative to the baseline MAP measurement. In some embodiments, the MAP measurement is decreased by about 20% or more, about 30% or more, about 40% or more, about 50% or more, about 60% or more, about 70% or more, about 80% or more, about 90% or more, relative to the baseline MAP measurement. In some embodiments, the MAP measurement is decreased by no more than about 2.5%, no more than about 5%, or no more than about 7.5%, relative to the baseline MAP measurement. In some embodiments, the MAP measurement is decreased by no more than about 10%, relative to the baseline MAP measurement. In some embodiments, the MAP measurement is decreased by no more than about 20%, no more than about 30%, no more than about 40%, no more than about 50%, no more than about 60%, no more than about 70%, no more than about 80%, or no more than about 90% relative to the baseline MAP measurement. In some embodiments, the MAP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a pulse pressure measurement. In some embodiments, the pulse pressure measurement is measured in mm of mercury (mm Hg). In some embodiments, the pulse pressure measurement is obtained with a sphygmomanometer. The pulse pressure measurement may be indicative of hypertension. The pulse pressure measurement may be indicative of normal blood pressure. The pulse pressure measurement may include a cerebral pulse pressure measurement.

In some embodiments, the composition reduces the pulse pressure measurement relative to the baseline pulse pressure measurement. In some embodiments, the reduction is measured directly in the subject after administering the composition to the subject. In some embodiments, the pulse pressure measurement is decreased by about 2.5% or more, about 5% or more, or about 7.5% or more, relative to the baseline pulse pressure measurement. In some embodiments, the pulse pressure measurement is decreased by about 10% or more, relative to the baseline pulse pressure measurement. In some embodiments, the pulse pressure measurement is decreased by about 20% or more, about 30% or more, about 40% or more, about 50% or more, about 60% or more, about 70% or more, about 80% or more, about 90% or more, relative to the baseline pulse pressure measurement. In some embodiments, the pulse pressure measurement is decreased by no more than about 2.5%, no more than about 5%, or no more than about 7.5%, relative to the baseline pulse pressure measurement. In some embodiments, the pulse pressure measurement is decreased by no more than about 10%, relative to the baseline pulse pressure measurement. In some embodiments, the pulse pressure measurement is decreased by no more than about 20%, no more than about 30%, no more than about 40%, no more than about 50%, no more than about 60%, no more than about 70%, no more than about 80%, or no more than about 90% relative to the baseline pulse pressure measurement. In some embodiments, the pulse pressure measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a intraocular pressure (IOP) measurement. The IOP may be measured using a tonometer. The IOP measurement may be in millimeters of mercury (mmHg). The IOP measurement may be indicative of a normal IOP. The IOP measurement may be indicative of abnormal or high IOP.

In some embodiments, the composition reduces the IOP measurement relative to the baseline IOP measurement. In some embodiments, the reduction is measured directly in the subject after administering the composition to the subject. In some embodiments, the IOP measurement is decreased by about 2.5% or more, about 5% or more, or about 7.5% or more, relative to the baseline IOP measurement. In some embodiments, the IOP measurement is decreased by about 10% or more, relative to the baseline IOP measurement. In some embodiments, the IOP measurement is decreased by about 20% or more, about 30% or more, about 40% or more, about 50% or more, about 60% or more, about 70% or more, about 80% or more, about 90% or more, relative to the baseline IOP measurement. In some embodiments, the IOP measurement is decreased by no more than about 2.5%, no more than about 5%, or no more than about 7.5%, relative to the baseline IOP measurement. In some embodiments, the IOP measurement is decreased by no more than about 10%, relative to the baseline IOP measurement. In some embodiments, the IOP measurement is decreased by no more than about 20%, no more than about 30%, no more than about 40%, no more than about 50%, no more than about 60%, no more than about 70%, no more than about 80%, or no more than about 90% relative to the baseline IOP measurement. In some embodiments, the IOP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a measurement of optic nerve head cupping. The measurement of optic nerve head cupping may be a cup-disc ratio measurement. The cup-disc ratio may be measured using a slit lamp. The cup-disc ratio measurement may be indicative of a normal cup-disc ratio. The cup-disc ratio measurement may be indicative of a high or abnormal cup-disc ratio.

In some embodiments, the composition reduces the cup-disc ratio measurement relative to the baseline cup-disc ratio measurement. In some embodiments, the reduction is measured directly in the subject after administering the composition to the subject. In some embodiments, the cup-disc ratio measurement is decreased by about 2.5% or more, about 5% or more, or about 7.5% or more, relative to the baseline cup-disc ratio measurement. In some embodiments, the cup-disc ratio measurement is decreased by about 10% or more, relative to the baseline cup-disc ratio measurement. In some embodiments, the cup-disc ratio measurement is decreased by about 20% or more, about 30% or more, about 40% or more, about 50% or more, about 60% or more, about 70% or more, about 80% or more, about 90% or more, relative to the baseline cup-disc ratio measurement. In some embodiments, the cup-disc ratio measurement is decreased by no more than about 2.5%, no more than about 5%, or no more than about 7.5%, relative to the baseline cup-disc ratio measurement. In some embodiments, the cup-disc ratio measurement is decreased by no more than about 10%, relative to the baseline cup-disc ratio measurement. In some embodiments, the cup-disc ratio measurement is decreased by no more than about 20%, no more than about 30%, no more than about 40%, no more than about 50%, no more than about 60%, no more than about 70%, no more than about 80%, or no more than about 90% relative to the baseline cup-disc ratio measurement. In some embodiments, the cup-disc ratio measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a retinal nerve fiber layer (RNFL) thickness measurement. The RNFL thickness may be measured using optical coherence tomography. The RNFL thickness measurement may be in μm. The RNFL thickness measurement may be indicative of a normal RNFL thickness measurement. The RNFL thickness measurement may be indicative of a low or abnormal RNFL thickness measurement.

In some embodiments, the composition increases the RNFL thickness measurement relative to the baseline RNFL thickness measurement. In some embodiments, the increase is measured directly in the subject after administering the composition to the subject. In some embodiments, the RNFL thickness measurement is increased by about 2.5% or more, about 5% or more, or about 7.5% or more, relative to the baseline RNFL thickness measurement. In some embodiments, the RNFL thickness measurement is increased by about 10% or more, relative to the baseline RNFL thickness measurement. In some embodiments, the RNFL thickness measurement is increased by about 20% or more, about 30% or more, about 40% or more, about 50% or more, about 60% or more, about 70% or more, about 80% or more, about 90% or more, relative to the baseline RNFL thickness measurement. In some embodiments, the RNFL thickness measurement is increased by no more than about 2.5%, no more than about 5%, or no more than about 7.5%, relative to the baseline RNFL thickness measurement. In some embodiments, the RNFL thickness measurement is increased by no more than about 10%, relative to the baseline RNFL thickness measurement. In some embodiments, the RNFL thickness measurement is increased by no more than about 20%, no more than about 30%, no more than about 40%, no more than about 50%, no more than about 60%, no more than about 70%, no more than about 80%, or no more than about 90% relative to the baseline RNFL thickness measurement. In some embodiments, the RNFL thickness measurement is increased by 2.5%, 5%, 7.5%, 10%, 20%, 30%, 40%, 50%, 60%, 70%, 80%, or 90%, or by a range defined by any of the two aforementioned percentages.

In some embodiments, the measurement is a retinal thickness measurement. The retinal thickness may be measured using optical coherence tomography. The retinal thickness measurement may be in m. The retinal thickness measurement may be indicative of a normal retinal thickness measurement. The retinal thickness measurement may be indicative of a high or abnormal retinal thickness measurement.

In some embodiments, the composition reduces the retinal thickness measurement relative to the baseline retinal thickness measurement. In some embodiments, the reduction is measured directly in the subject after administering the composition to the subject. In some embodiments, the retinal thickness measurement is decreased by about 2.5% or more, about 5% or more, or about 7.5% or more, relative to the baseline retinal thickness measurement. In some embodiments, the retinal thickness measurement is decreased by about 10% or more, relative to the baseline retinal thickness measurement. In some embodiments, the retinal thickness measurement is decreased by about 20% or more, about 30% or more, about 40% or more, about 50% or more, about 60% or more, about 70% or more, about 80% or more, about 90% or more, relative to the baseline retinal thickness measurement. In some embodiments, the retinal thickness measurement is decreased by no more than about 2.5%, no more than about 5%, or no more than about 7.5%, relative to the baseline retinal thickness measurement. In some embodiments, the retinal thickness measurement is decreased by no more than about 10%, relative to the baseline retinal thickness measurement. In some embodiments, the retinal thickness measurement is decreased by no more than about 20%, no more than about 30%, no more than about 40%, no more than about 50%, no more than about 60%, no more than about 70%, no more than about 80%, or no more than about 90% relative to the baseline retinal thickness measurement. In some embodiments, the retinal thickness measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a edema measurement. The edema may be measured using optical coherence tomography. The edema measurement may be in μm. The edema measurement may be indicative of a normal edema measurement. The edema measurement may be indicative of a high or abnormal edema measurement.

In some embodiments, the composition reduces the edema measurement relative to the baseline edema measurement. In some embodiments, the reduction is measured directly in the subject after administering the composition to the subject. In some embodiments, the edema measurement is decreased by about 2.5% or more, about 5% or more, or about 7.5% or more, relative to the baseline edema measurement. In some embodiments, the edema measurement is decreased by about 10% or more, relative to the baseline edema measurement. In some embodiments, the edema measurement is decreased by about 20% or more, about 30% or more, about 40% or more, about 50% or more, about 60% or more, about 70% or more, about 80% or more, about 90% or more, relative to the baseline edema measurement. In some embodiments, the edema measurement is decreased by no more than about 2.5%, no more than about 5%, or no more than about 7.5%, relative to the baseline edema measurement. In some embodiments, the edema measurement is decreased by no more than about 10%, relative to the baseline edema measurement. In some embodiments, the edema measurement is decreased by no more than about 20%, no more than about 30%, no more than about 40%, no more than about 50%, no more than about 60%, no more than about 70%, no more than about 80%, or no more than about 90% relative to the baseline edema measurement. In some embodiments, the edema measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a RPE pigmentation and reflectivity measurement. The RPE pigmentation and reflectivity measurement may be measured using optical coherence tomography. The RPE pigmentation and reflectivity measurement may be indicative of a normal RPE pigmentation and reflectivity measurement. The RPE pigmentation and reflectivity measurement may be indicative of abnormal RPE pigmentation and reflectivity.

In some embodiments, the composition reduces the RPE pigmentation and reflectivity measurement relative to the baseline RPE pigmentation and reflectivity measurement. In some embodiments, the reduction is measured directly in the subject after administering the composition to the subject. In some embodiments, the RPE pigmentation and reflectivity measurement is decreased by about 2.5% or more, about 5% or more, or about 7.5% or more, relative to the baseline RPE pigmentation and reflectivity measurement. In some embodiments, the RPE pigmentation and reflectivity measurement is decreased by about 10% or more, relative to the baseline RPE pigmentation and reflectivity measurement. In some embodiments, the RPE pigmentation and reflectivity measurement is decreased by about 20% or more, about 30% or more, about 40% or more, about 50% or more, about 60% or more, about 70% or more, about 80% or more, about 90% or more, relative to the baseline RPE pigmentation and reflectivity measurement. In some embodiments, the RPE pigmentation and reflectivity measurement is decreased by no more than about 2.5%, no more than about 5%, or no more than about 7.5%, relative to the baseline RPE pigmentation and reflectivity measurement. In some embodiments, the RPE pigmentation and reflectivity measurement is decreased by no more than about 10%, relative to the baseline RPE pigmentation and reflectivity measurement. In some embodiments, the RPE pigmentation and reflectivity measurement is decreased by no more than about 20%, no more than about 30%, no more than about 40%, no more than about 50%, no more than about 60%, no more than about 70%, no more than about 80%, or no more than about 90% relative to the baseline RPE pigmentation and reflectivity measurement. In some embodiments, the RPE pigmentation and reflectivity measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a drusen measurement. The drusen may be measured using an eye exam or retinal photography. The drusen measurement may be the size of the drusen or the number of the drusen. The drusen measurement may be indicative of a normal drusen measurement. The drusen measurement may be indicative of a high or abnormal drusen measurement.

In some embodiments, the composition reduces the drusen measurement relative to the baseline drusen measurement. In some embodiments, the reduction is measured directly in the subject after administering the composition to the subject. In some embodiments, the drusen measurement is decreased by about 2.5% or more, about 5% or more, or about 7.5% or more, relative to the baseline drusen measurement. In some embodiments, the drusen measurement is decreased by about 10% or more, relative to the baseline drusen measurement. In some embodiments, the drusen measurement is decreased by about 20% or more, about 30% or more, about 40% or more, about 50% or more, about 60% or more, about 70% or more, about 80% or more, about 90% or more, relative to the baseline drusen measurement. In some embodiments, the drusen measurement is decreased by no more than about 2.5%, no more than about 5%, or no more than about 7.5%, relative to the baseline drusen measurement. In some embodiments, the drusen measurement is decreased by no more than about 10%, relative to the baseline drusen measurement. In some embodiments, the drusen measurement is decreased by no more than about 20%, no more than about 30%, no more than about 40%, no more than about 50%, no more than about 60%, no more than about 70%, no more than about 80%, or no more than about 90% relative to the baseline drusen measurement. In some embodiments, the drusen measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a hemorrhage measurement. The hemorrhage measurement may be a vitreous hemorrhage. The hemorrhage measurement may be an intraretinal hemorrhage. The hemorrhage may be a macular hemorrhage. The hemorrhage may be measured using retinal photography. The hemorrhage measurement may be indicative of a normal hemorrhage measurement. The hemorrhage measurement may be indicative of an abnormal or high hemorrhage measurement.

In some embodiments, the composition reduces the hemorrhage measurement relative to the baseline hemorrhage measurement. In some embodiments, the reduction is measured directly in the subject after administering the composition to the subject. In some embodiments, the hemorrhage measurement is decreased by about 2.5% or more, about 5% or more, or about 7.5% or more, relative to the baseline hemorrhage measurement. In some embodiments, the hemorrhage measurement is decreased by about 10% or more, relative to the baseline hemorrhage measurement. In some embodiments, the hemorrhage measurement is decreased by about 20% or more, about 30% or more, about 40% or more, about 50% or more, about 60% or more, about 70% or more, about 80% or more, about 90% or more, relative to the baseline hemorrhage measurement. In some embodiments, the hemorrhage measurement is decreased by no more than about 2.5%, no more than about 5%, or no more than about 7.5%, relative to the baseline hemorrhage measurement. In some embodiments, the hemorrhage measurement is decreased by no more than about 10%, relative to the baseline hemorrhage measurement. In some embodiments, the hemorrhage measurement is decreased by no more than about 20%, no more than about 30%, no more than about 40%, no more than about 50%, no more than about 60%, no more than about 70%, no more than about 80%, or no more than about 90% relative to the baseline hemorrhage measurement. In some embodiments, the hemorrhage measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a macular ischemia measurement. The macular ischemia may be measured using fluorescein angiography or optical coherence tomography angiography. The macular ischemia measurement may be a measurement of the foveal avascular zone. The macular ischemia measurement may be indicative of a normal macular ischemia measurement. The macular ischemia measurement may be indicative of a high or abnormal macular ischemia measurement.

In some embodiments, the composition reduces the macular ischemia measurement relative to the baseline macular ischemia measurement. In some embodiments, the reduction is measured directly in the subject after administering the composition to the subject. In some embodiments, the macular ischemia measurement is decreased by about 2.5% or more, about 5% or more, or about 7.5% or more, relative to the baseline macular ischemia measurement. In some embodiments, the macular ischemia measurement is decreased by about 10% or more, relative to the baseline macular ischemia measurement. In some embodiments, the macular ischemia measurement is decreased by about 20% or more, about 30% or more, about 40% or more, about 50% or more, about 60% or more, about 70% or more, about 80% or more, about 90% or more, relative to the baseline macular ischemia measurement. In some embodiments, the macular ischemia measurement is decreased by no more than about 2.5%, no more than about 5%, or no more than about 7.5%, relative to the baseline macular ischemia measurement. In some embodiments, the macular ischemia measurement is decreased by no more than about 10%, relative to the baseline macular ischemia measurement. In some embodiments, the macular ischemia measurement is decreased by no more than about 20%, no more than about 30%, no more than about 40%, no more than about 50%, no more than about 60%, no more than about 70%, no more than about 80%, or no more than about 90% relative to the baseline macular ischemia measurement. In some embodiments, the macular ischemia measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a microaneurysm measurement. The microaneurysm may be measured using fluorescein angiography. The microaneurysm measurement may be the count of microaneurysms. The microaneurysm measurement may be indicative of a normal microaneurysm measurement. The microaneurysm measurement may be indicative of high or abnormal microaneurysm measurement.

In some embodiments, the composition reduces the microaneurysm measurement relative to the baseline microaneurysm measurement. In some embodiments, the reduction is measured directly in the subject after administering the composition to the subject. In some embodiments, the microaneurysm measurement is decreased by about 2.5% or more, about 5% or more, or about 7.5% or more, relative to the baseline microaneurysm measurement. In some embodiments, the microaneurysm measurement is decreased by about 10% or more, relative to the baseline microaneurysm measurement. In some embodiments, the microaneurysm measurement is decreased by about 20% or more, about 30% or more, about 40% or more, about 50% or more, about 60% or more, about 70% or more, about 80% or more, about 90% or more, relative to the baseline microaneurysm measurement. In some embodiments, the microaneurysm measurement is decreased by no more than about 2.5%, no more than about 5%, or no more than about 7.5%, relative to the baseline microaneurysm measurement. In some embodiments, the microaneurysm measurement is decreased by no more than about 10%, relative to the baseline microaneurysm measurement. In some embodiments, the microaneurysm measurement is decreased by no more than about 20%, no more than about 30%, no more than about 40%, no more than about 50%, no more than about 60%, no more than about 70%, no more than about 80%, or no more than about 90% relative to the baseline microaneurysm measurement. In some embodiments, the microaneurysm measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a neovascularization measurement. The neovascularization measurement may be a choroidal neovascularization measurement. The neovascularization measurement may be measured using imaging techniques such as fluorescein angiography. The neovascularization measurement may be an area of the neovascularization. The neovascularization measurement may be indicative of a normal neovascularization measurement. The neovascularization measurement may be indicative of a high or abnormal neovascularization measurement.

In some embodiments, the composition reduces the neovascularization measurement relative to the baseline neovascularization measurement. In some embodiments, the reduction is measured directly in the subject after administering the composition to the subject. In some embodiments, the neovascularization measurement is decreased by about 2.5% or more, about 5% or more, or about 7.5% or more, relative to the baseline neovascularization measurement. In some embodiments, the neovascularization measurement is decreased by about 10% or more, relative to the baseline neovascularization measurement. In some embodiments, the neovascularization measurement is decreased by about 20% or more, about 30% or more, about 40% or more, about 50% or more, about 60% or more, about 70% or more, about 80% or more, about 90% or more, relative to the baseline neovascularization measurement. In some embodiments, the neovascularization measurement is decreased by no more than about 2.5%, no more than about 5%, or no more than about 7.5%, relative to the baseline neovascularization measurement. In some embodiments, the neovascularization measurement is decreased by no more than about 10%, relative to the baseline neovascularization measurement. In some embodiments, the neovascularization measurement is decreased by no more than about 20%, no more than about 30%, no more than about 40%, no more than about 50%, no more than about 60%, no more than about 70%, no more than about 80%, or no more than about 90% relative to the baseline neovascularization measurement. In some embodiments, the neovascularization measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a traction retinal detachment measurement. The traction retinal detachment may be measured using imaging techniques, including optical coherence tomography. The traction retinal detachment measurement may be indicative of a normal retina. The traction retinal detachment measurement may be indicative of macular degeneration or diabetic retinopathy.

In some embodiments, the composition reduces the traction retinal detachment measurement relative to the baseline traction retinal detachment measurement. In some embodiments, the reduction is measured directly in the subject after administering the composition to the subject. In some embodiments, the traction retinal detachment measurement is decreased by about 2.5% or more, about 5% or more, or about 7.5% or more, relative to the baseline traction retinal detachment measurement. In some embodiments, the traction retinal detachment measurement is decreased by about 10% or more, relative to the baseline traction retinal detachment measurement. In some embodiments, the traction retinal detachment measurement is decreased by about 20% or more, about 30% or more, about 40% or more, about 50% or more, about 60% or more, about 70% or more, about 80% or more, about 90% or more, relative to the baseline traction retinal detachment measurement. In some embodiments, the traction retinal detachment measurement is decreased by no more than about 2.5%, no more than about 5%, or no more than about 7.5%, relative to the baseline traction retinal detachment measurement. In some embodiments, the traction retinal detachment measurement is decreased by no more than about 10%, relative to the baseline traction retinal detachment measurement. In some embodiments, the traction retinal detachment measurement is decreased by no more than about 20%, no more than about 30%, no more than about 40%, no more than about 50%, no more than about 60%, no more than about 70%, no more than about 80%, or no more than about 90% relative to the baseline traction retinal detachment measurement. In some embodiments, the traction retinal detachment measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a hemoglobin A1C measurement. In some embodiments, the hemoglobin A1C measurement is a hemoglobin A1C concentration. In some embodiments, the hemoglobin A1C measurement is a circulating hemoglobin A1C measurement. In some embodiments, the hemoglobin A1C measurement is obtained by an assay such as an immunoassay, a colorimetric assay, a fluorescence assay, or HPLC. The hemoglobin A1C measurement may be indicative of a healthy normal A1C measurement. The hemoglobin A1C measurement may be indicative of diabetes. The hemoglobin A1C measurement may be indicative of pre-diabetes.

In some embodiments, the composition reduces the hemoglobin A1C measurement relative to the baseline hemoglobin Ab C measurement. In some embodiments, the reduction is measured in a second fluid sample obtained from the subject after administering the composition to the subject. In some embodiments, the reduction is measured directly in the subject after administering the composition to the subject. In some embodiments, the hemoglobin A1C measurement is decreased by about 2.5% or more, about 50% or more, or about 7.50% or more, relative to the baseline hemoglobin A1C measurement. In some embodiments, the hemoglobin A1C measurement is decreased by about 10% or more, relative to the baseline hemoglobin A1C measurement. In some embodiments, the hemoglobin A1C measurement is decreased by about 20% or more, about 30% or more, about 40% or more, about 50% or more, about 60% or more, about 70% or more, about 80% or more, about 90% or more, relative to the baseline hemoglobin A1C measurement. In some embodiments, the hemoglobin A1C measurement is decreased by no more than about 2.5%, no more than about 5%, or no more than about 7.5%, relative to the baseline hemoglobin A1C measurement. In some embodiments, the hemoglobin A1C measurement is decreased by no more than about 10%, relative to the baseline hemoglobin A1C measurement. In some embodiments, the hemoglobin A1C measurement is decreased by no more than about 20%, no more than about 30%, no more than about 40%, no more than about 50%, no more than about 60%, no more than about 70%, no more than about 80%, or no more than about 90% relative to the baseline hemoglobin A1C measurement. In some embodiments, the hemoglobin A1C measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a body mass measurement. In some embodiments, the body mass measurement is a body mass index (BMI). BMI may be defined as a body mass divided by the square of body height, and may be expressed in units of kg/m². Body mass may be obtained using a scale. Body height may be measured using a ruler or a measuring tape. Body height may include the height of a standing subject. Body height may include a distance from the bottom of a subject's feet to the top of the subject's head. BMI may include BMI prime. The subject may have a BMI in a range exemplified in Table 3.

In some embodiments, the composition reduces the body mass measurement relative to the baseline body mass measurement. In some embodiments, the reduction is measured on the subject after administering the composition to the subject. In some embodiments, the body mass measurement is decreased by about 2.5% or more, about 5% or more, or about 7.5% or more, relative to the baseline body mass measurement. In some embodiments, the body mass measurement is decreased by about 10% or more, relative to the baseline body mass measurement. In some embodiments, the body mass measurement is decreased by about 20% or more, about 30% or more, about 40% or more, about 50% or more, about 60% or more, about 70% or more, about 80% or more, about 90% or more, relative to the baseline body mass measurement. In some embodiments, the body mass measurement is decreased by no more than about 2.5%, no more than about 5%, or no more than about 7.5%, relative to the baseline body mass measurement. In some embodiments, the body mass measurement is decreased by no more than about 10%, relative to the baseline body mass measurement. In some embodiments, the body mass measurement is decreased by no more than about 20%, no more than about 30%, no more than about 40%, no more than about 50%, no more than about 60%, no more than about 70%, no more than about 80%, or no more than about 90% relative to the baseline body mass measurement. In some embodiments, the body mass measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a waist circumference measurement. A waist circumference measurement may be obtained using a measuring tape.

In some embodiments, the composition reduces the waist circumference measurement relative to the baseline waist circumference measurement. In some embodiments, the reduction is measured on the subject after administering the composition to the subject. In some embodiments, the waist circumference measurement is decreased by about 2.5% or more, about 5% or more, or about 7.5% or more, relative to the baseline waist circumference measurement. In some embodiments, the waist circumference measurement is decreased by about 10% or more, relative to the baseline waist circumference measurement. In some embodiments, the waist circumference measurement is decreased by about 20% or more, about 30% or more, about 40% or more, about 50% or more, about 60% or more, about 70% or more, about 80% or more, about 90% or more, relative to the baseline waist circumference measurement. In some embodiments, the waist circumference measurement is decreased by no more than about 2.5%, no more than about 5%, or no more than about 7.5%, relative to the baseline waist circumference measurement. In some embodiments, the waist circumference measurement is decreased by no more than about 10%, relative to the baseline waist circumference measurement. In some embodiments, the waist circumference measurement is decreased by no more than about 20%, no more than about 30%, no more than about 40%, no more than about 50%, no more than about 60%, no more than about 70%, no more than about 80%, or no more than about 90% relative to the baseline waist circumference measurement. In some embodiments, the waist circumference measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a hip circumference measurement. A hip circumference measurement may be obtained using a measuring tape.

In some embodiments, the composition reduces the hip circumference measurement relative to the baseline hip circumference measurement. In some embodiments, the reduction is measured on the subject after administering the composition to the subject. In some embodiments, the hip circumference measurement is decreased by about 2.5% or more, about 5% or more, or about 7.5% or more, relative to the baseline hip circumference measurement. In some embodiments, the hip circumference measurement is decreased by about 10% or more, relative to the baseline hip circumference measurement. In some embodiments, the hip circumference measurement is decreased by about 20% or more, about 30% or more, about 40% or more, about 50% or more, about 60% or more, about 70% or more, about 80% or more, about 90% or more, relative to the baseline hip circumference measurement. In some embodiments, the hip circumference measurement is decreased by no more than about 2.5%, no more than about 5%, or no more than about 7.5%, relative to the baseline hip circumference measurement. In some embodiments, the hip circumference measurement is decreased by no more than about 10%, relative to the baseline hip circumference measurement. In some embodiments, the hip circumference measurement is decreased by no more than about 20%, no more than about 30%, no more than about 40%, no more than about 50%, no more than about 60%, no more than about 70%, no more than about 80%, or no more than about 90% relative to the baseline hip circumference measurement. In some embodiments, the hip circumference measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a waist-hip ratio. A waist-hip ratio may be obtained using a measuring tape.

In some embodiments, the composition reduces the waist-hip ratio measurement relative to the baseline waist-hip ratio measurement. In some embodiments, the reduction is measured on the subject after administering the composition to the subject. In some embodiments, the waist-hip ratio measurement is decreased by about 2.5% or more, about 5% or more, or about 7.5% or more, relative to the baseline waist-hip ratio measurement. In some embodiments, the waist-hip ratio measurement is decreased by about 10% or more, relative to the baseline waist-hip ratio measurement. In some embodiments, the waist-hip ratio measurement is decreased by about 20% or more, about 30% or more, about 40% or more, about 50% or more, about 60% or more, about 70% or more, or about 80% or more, relative to the baseline waist-hip ratio measurement. In some embodiments, the waist-hip ratio measurement is decreased by no more than about 2.5%, no more than about 5%, or no more than about 7.5%, relative to the baseline waist-hip ratio measurement. In some embodiments, the waist-hip ratio measurement is decreased by no more than about 10%, relative to the baseline waist-hip ratio measurement. In some embodiments, the waist-hip ratio measurement is decreased by no more than about 20%, no more than about 30%, no more than about 40%, no more than about 50%, no more than about 60%, no more than about 70%, or no more than about 80%, relative to the baseline waist-hip ratio measurement. In some embodiments, the waist-hip ratio measurement is decreased by 2.5%, 5%, 7.5%, 10%, 20%, 30%, 40%, 50%, 60%, 70%, or 80%, or by a range defined by any of the two aforementioned percentages.

In some embodiments, the measurement is a body fat percentage. A body fat percentage may be obtained using underwater weighing, whole-body air displacement plethysmography, near-infrared interactance, dual energy X-ray absorptiometry, bioelectrical impedance, or a skinfold test.

In some embodiments, the composition reduces the body fat percentage measurement relative to the baseline body fat percentage measurement. In some embodiments, the reduction is measured on the subject after administering the composition to the subject. In some embodiments, the body fat percentage measurement is decreased by about 2.5% or more, about 5% or more, or about 7.5% or more, relative to the baseline body fat percentage measurement. In some embodiments, the body fat percentage measurement is decreased by about 10% or more, relative to the baseline body fat percentage measurement. In some embodiments, the body fat percentage measurement is decreased by about 20% or more, about 30% or more, about 40% or more, about 50% or more, about 60% or more, about 70% or more, about 80% or more, about 90% or more, relative to the baseline body fat percentage measurement. In some embodiments, the body fat percentage measurement is decreased by no more than about 2.5%, no more than about 5%, or no more than about 7.5%, relative to the baseline body fat percentage measurement. In some embodiments, the body fat percentage measurement is decreased by no more than about 10%, relative to the baseline body fat percentage measurement. In some embodiments, the body fat percentage measurement is decreased by no more than about 20%, no more than about 30%, no more than about 40%, no more than about 50%, no more than about 60%, no more than about 70%, no more than about 80%, or no more than about 90% relative to the baseline body fat percentage measurement. In some embodiments, the body fat percentage measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a triglyceride measurement. In some embodiments, the triglyceride measurement is a triglyceride concentration (for example, mg/dL). In some embodiments, the triglyceride measurement is a circulating triglyceride measurement. In some embodiments, the triglyceride measurement is obtained by an assay such as an immunoassay, a colorimetric assay, or a fluorescence assay.

In some embodiments, the composition reduces the triglyceride measurement relative to the baseline triglyceride measurement. In some embodiments, the composition reduces circulating triglycerides relative to the baseline triglyceride measurement. In some embodiments, the reduced triglycerides are measured in a second sample obtained from the subject after administering the composition to the subject. In some embodiments, the triglyceride measurement is decreased by about 2.5% or more, about 5% or more, or about 7.5% or more, relative to the baseline triglyceride measurement. In some embodiments, the triglyceride measurement is decreased by about 10% or more, relative to the baseline triglyceride measurement. In some embodiments, the triglyceride measurement is decreased by about 20% or more, about 30% or more, about 40% or more, about 50% or more, about 60% or more, about 70% or more, about 80% or more, about 90% or more, or about 100%, relative to the baseline triglyceride measurement. In some embodiments, the triglyceride measurement is decreased by no more than about 2.5%, no more than about 5%, or no more than about 7.5%, relative to the baseline triglyceride measurement. In some embodiments, the triglyceride measurement is decreased by no more than about 10%, relative to the baseline triglyceride measurement. In some embodiments, the triglyceride measurement is decreased by no more than about 20%, no more than about 30%, no more than about 40%, no more than about 50%, no more than about 60%, no more than about 70%, no more than about 80%, no more than about 90%, or no more than about 100% relative to the baseline triglyceride measurement. In some embodiments, the triglyceride measurement is decreased by 2.5%, 5%, 7.5%, 10%, 20%, 30%, 40%, 50%, 60%, 70%, 80%, 90%, 100%, or by a range defined by any of the two aforementioned percentages.

In some embodiments, the measurement is a cholesterol measurement. In some embodiments, the cholesterol concentration is a total cholesterol measurement. In some embodiments, the cholesterol concentration is a non-high density lipoprotein (HDL) cholesterol measurement. In some embodiments, the cholesterol concentration is a low density lipoprotein (LDL) cholesterol measurement. In some embodiments, the cholesterol measurement is a cholesterol concentration. In some embodiments, the cholesterol measurement is a circulating cholesterol measurement. In some embodiments, the cholesterol measurement is a blood cholesterol measurement. In some embodiments, the cholesterol measurement is obtained by an assay such as an immunoassay, a colorimetric assay, or a fluorescence assay.

In some embodiments, the composition reduces the cholesterol measurement relative to the baseline cholesterol measurement. In some embodiments, the reduction is measured in a second sample obtained from the subject after administering the composition to the subject. In some embodiments, the cholesterol measurement is decreased by about 2.5% or more, about 5% or more, or about 7.5% or more, relative to the baseline cholesterol measurement. In some embodiments, the cholesterol measurement is decreased by about 10% or more, relative to the baseline cholesterol measurement. In some embodiments, the cholesterol measurement is decreased by about 20% or more, about 30% or more, about 40% or more, about 50% or more, about 60% or more, about 70% or more, about 80% or more, about 90% or more, relative to the baseline cholesterol measurement. In some embodiments, the cholesterol measurement is decreased by no more than about 2.5%, no more than about 5%, or no more than about 7.5%, relative to the baseline cholesterol measurement. In some embodiments, the cholesterol measurement is decreased by no more than about 10%, relative to the baseline cholesterol measurement. In some embodiments, the cholesterol measurement is decreased by no more than about 20%, no more than about 30%, no more than about 40%, no more than about 50%, no more than about 60%, no more than about 70%, no more than about 80%, or no more than about 90%, relative to the baseline cholesterol measurement. In some embodiments, the cholesterol measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a liver enzyme measurement. In some embodiments, the liver enzyme measurement is an alanine aminotransferase (ALT) measurement. In some embodiments, the liver enzyme measurement is an aspartate aminotransferase (AST) measurement. In some embodiments, the liver enzyme measurement comprises an ALT/AST ratio, or comprises an AST/ALT ratio.

In some embodiments, the measurement is a alanine aminotransferase (ALT) measurement. In some embodiments, the ALT measurement is a ALT concentration (for example, Units/dL). In some embodiments, the ALT measurement is a circulating ALT measurement, for example, a blood, serum, or plasma ALT measurement. In some embodiments, the ALT measurement is obtained by an assay such as an immunoassay, a colorimetric assay, or a fluorescence assay. In some embodiments, the ALT measurement is within a reference range. In some embodiments, the ALT measurement is above the reference range.

In some embodiments, the composition reduces the ALT measurement relative to the baseline ALT measurement. In some embodiments, the reduced ALT is measured in a second blood sample, plasma sample, or serum sample obtained from the subject after administering the composition to the subject. In some embodiments, the ALT measurement is decreased by about 2.5% or more, about 5% or more, or about 7.5% or more, relative to the baseline ALT measurement. In some embodiments, the ALT measurement is decreased by about 10% or more, relative to the baseline ALT measurement. In some embodiments, the ALT measurement is decreased by about 20% or more, about 30% or more, about 40% or more, about 50% or more, about 60% or more, about 70% or more, about 80% or more, about 90% or more, relative to the baseline ALT measurement. In some embodiments, the ALT measurement is decreased by no more than about 2.5%, no more than about 5%, or no more than about 7.5%, relative to the baseline ALT measurement. In some embodiments, the ALT measurement is decreased by no more than about 10%, relative to the baseline ALT measurement. In some embodiments, the ALT measurement is decreased by no more than about 20%, no more than about 30%, no more than about 40%, no more than about 50%, no more than about 60%, no more than about 70%, no more than about 80%, or no more than about 90% relative to the baseline ALT measurement. In some embodiments, the ALT measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a aspartate aminotransferase (AST) measurement. In some embodiments, the AST measurement is a AST concentration (for example, Units/L). In some embodiments, the AST measurement is a circulating AST measurement, for example, a blood, serum, or plasma AST measurement. In some embodiments, the AST measurement is obtained by an assay such as an immunoassay, a colorimetric assay, or a fluorescence assay. In some embodiments, the AST measurement is within a reference range. In some embodiments, the AST measurement is above the reference range. In some embodiments, the AST measurement is below the reference range.

In some embodiments, the composition reduces the AST measurement relative to the baseline AST measurement. In some embodiments, the reduced AST is measured in a second blood sample, plasma sample, or serum sample obtained from the subject after administering the composition to the subject. In some embodiments, the AST measurement is decreased by about 2.5% or more, about 5% or more, or about 7.5% or more, relative to the baseline AST measurement. In some embodiments, the AST measurement is decreased by about 10% or more, relative to the baseline AST measurement. In some embodiments, the AST measurement is decreased by about 20% or more, about 30% or more, about 40% or more, about 50% or more, about 60% or more, about 70% or more, about 80% or more, about 90% or more, relative to the baseline AST measurement. In some embodiments, the AST measurement is decreased by no more than about 2.5%, no more than about 5%, or no more than about 7.5%, relative to the baseline AST measurement. In some embodiments, the AST measurement is decreased by no more than about 10%, relative to the baseline AST measurement. In some embodiments, the AST measurement is decreased by no more than about 20%, no more than about 30%, no more than about 40%, no more than about 50%, no more than about 60%, no more than about 70%, no more than about 80%, no more than about 90% relative to the baseline AST measurement. In some embodiments, the AST measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a liver steatosis measurement. In some embodiments, the liver steatosis measurement is a liver fat percentage (LFP) measurement. In some embodiments, the measurement is a LFP measurement. In some embodiments, the LFP measurement is indicated as a mass/mass percentage of fat/total tissue. In some embodiments, the LFP measurement is indicated as a mass/volume percentage of fat/total tissue. In some embodiments, the LFP measurement is indicated as a volume/mass percentage of fat/total tissue. In some embodiments, the LFP measurement is indicated as a volume/volume percentage of fat/total tissue. In some embodiments, the LFP measurement is indicated as a score. In some embodiments, the LFP measurement is obtained noninvasively. In some embodiments, the LFP measurement is obtained by a medical imaging device. In some embodiments, the LFP measurement is obtained by a device such as a medical resonance imaging (MRI) device, a magnetic resonance spectroscopy device, a computed tomography device, a controlled attenuation parameter (CAP), a transient elastography device, or an ultrasound device. In some embodiments, the LFP measurement is obtained in a second liver sample. In some embodiments, the LFP measurement comprises a liver triglyceride measurement. In some embodiments, the LFP measurement is obtained by an assay such as an immunoassay, a colorimetric assay, or a fluorescence assay. In some embodiments, the LFP measurement or the LFP measurement is obtained using a scoring system upon a visual inspection of a sample such as a histological sample. In some embodiments, the LFP measurement or the LFP measurement is obtained using a stain with an affinity to fats, such as a lysochrome diazo dye.

In some embodiments, the composition reduces the LFP measurement relative to the baseline LFP measurement. In some embodiments, the reduced LFP is measured in a second liver sample obtained from the subject after administering the composition to the subject. In some embodiments, the reduced LFP is measured directly in the subject after administering the composition to the subject. In some embodiments, the LFP measurement is decreased by about 2.5% or more, about 5% or more, or about 7.5% or more, relative to the baseline LFP measurement. In some embodiments, the LFP measurement is decreased by about 10% or more, relative to the baseline LFP measurement. In some embodiments, the LFP measurement is decreased by about 20% or more, about 30% or more, about 40% or more, about 50% or more, about 60% or more, about 70% or more, about 80% or more, about 90% or more, relative to the baseline LFP measurement. In some embodiments, the LFP measurement is decreased by no more than about 2.5%, no more than about 5%, or no more than about 7.5%, relative to the baseline LFP measurement. In some embodiments, the LFP measurement is decreased by no more than about 10%, relative to the baseline LFP measurement. In some embodiments, the LFP measurement is decreased by no more than about 20%, no more than about 30%, no more than about 40%, no more than about 50%, no more than about 60%, no more than about 70%, no more than about 80%, or no more than about 90% relative to the baseline LFP measurement. In some embodiments, the LFP measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a liver fibrosis measurement. In some embodiments, the liver fibrosis measurement is a liver fibrosis score (LFS). In some embodiments, the LFS comprises a score of 0, 1, 2, 3, or 4, or a range of scores defined by any two of the aforementioned numbers. In some embodiments, the LFS comprises a score of 0-4. In some embodiments, the LFS is obtained using a scoring system exemplified in Table 4. In some embodiments, the LFS measurement is obtained noninvasively. In some embodiments, the LFS measurement is obtained by a medical imaging device such as a vibration-controlled transient elastography (VCTE) device, a shear wave elastography device, a medical resonance imaging (MRI) device, a magnetic resonance spectroscopy device, a computed tomography device, or an ultrasound device. In some embodiments, the LFS measurement is obtained in a second liver sample. In some embodiments, the LFS is obtained by an assay such as an immunoassay, a colorimetric assay, or a fluorescence assay. In some embodiments, the LFS is obtained using one or more indirect markers or measures of liver fibrosis such as an aspartate aminotransferase-to-platelet ratio index (APRI), a Fibrosis-4 (FIB-4) index, a FibroIndex, a Foms Index, a Hepascore, or a FibroTest. In some embodiments, the LFS is obtained using one or more indirect markers or measures of liver fibrosis such as a FIBROSpect test or a FIBROSpect II test. In some embodiments, the LFS is obtained by RT-qPCR or RNA sequencing of one or more fibrosis-related genes such as a collagen gene. In some embodiments, the LFS or the LFS is obtained using a scoring system upon a visual inspection of a sample such as a histological sample. In some embodiments, the LFS or the LFS is obtained using a stain with an affinity to collagen.

In some embodiments, the composition reduces the LFS relative to the baseline LFS. In some embodiments, the reduced LFS is measured in a second liver sample obtained from the subject after administering the composition to the subject. In some embodiments, the reduced LFS is measured directly in the subject after administering the composition to the subject. In some embodiments, the LFS is decreased by 1 relative to the baseline LFS. In some embodiments, the LFS is decreased by 2 relative to the baseline LFS. In some embodiments, the LFS is decreased by 3 relative to the baseline LFS. In some embodiments, the LFS is decreased by 4 relative to the baseline LFS. In some embodiments, the LFS is decreased by 1 or more, relative to the baseline LFS. In some embodiments, the LFS is decreased by 2 or more, relative to the baseline LFS. In some embodiments, the LFS is decreased by 3 more, relative to the baseline LFS. In some embodiments, the LFS is decreased by no more than 1, relative to the baseline LFS. In some embodiments, the LFS is decreased by no more than 2, relative to the baseline LFS. In some embodiments, the LFS is decreased by no more than 3, relative to the baseline LFS. In some embodiments, the LFS is decreased by no more than 4, relative to the baseline LFS. In some embodiments, the LFS is decreased by 1, 2, 3, or 4, or by a range defined by any of the two aforementioned numbers.

In some embodiments, the liver fibrosis measurement is a nonalcoholic fatty liver disease (NAFLD) fibrosis score. A NAFLD fibrosis score may take into account laboratory test values such as platelet count, albumin, and AST/ALT ratio, and patient characteristics such as BMI, and diabetes status. A NAFLD fibrosis score below −1.455 may be indicative of no fibrosis, mild fibrosis, or moderate fibrosis. A NAFLD fibrosis score between −1.455 and 0.675 may be indicative of severe fibrosis. A NAFLD fibrosis score above 0.675 may be indicative of cirrhosis.

In some embodiments, the composition reduces the NAFLD fibrosis score relative to the baseline NAFLD fibrosis score. In some embodiments, the NAFLD fibrosis score is decreased by about 2.5% or more, about 5% or more, or about 7.5% or more, relative to the baseline NAFLD fibrosis score. In some embodiments, the NAFLD fibrosis score is decreased by about 10% or more, relative to the baseline NAFLD fibrosis score. In some embodiments, the NAFLD fibrosis score is decreased by about 20% or more, about 30% or more, about 40% or more, about 50% or more, about 60% or more, about 70% or more, about 80% or more, about 90% or more, relative to the baseline NAFLD fibrosis score. In some embodiments, the NAFLD fibrosis score is decreased by no more than about 2.5%, no more than about 5%, or no more than about 7.5%, relative to the baseline NAFLD fibrosis score. In some embodiments, the NAFLD fibrosis score is decreased by no more than about 10%, relative to the baseline NAFLD fibrosis score. In some embodiments, the NAFLD fibrosis score is decreased by no more than about 20%, no more than about 30%, no more than about 40%, no more than about 50%, no more than about 60%, no more than about 70%, no more than about 80%, no more than about 90%, or no more than about 100% relative to the baseline NAFLD fibrosis score. In some embodiments, the NAFLD fibrosis score is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a non-alcoholic fatty liver disease (NAFLD) activity score. In some embodiments, the NAFLD activity score comprises a numerical value such as a number of points. In some embodiments, the numerical value is 0, 1, 2, 3, 4, 5, 6, 7, or 8, or a range defined by any two of the aforementioned numerical values. In some embodiments, the numerical value is 0-8. In some embodiments, the NAFLD activity score comprises a steatosis grade such as a liver fat percentage. In some embodiments, a steatosis grade <5% comprises 0 points in the NAFLD activity score. In some embodiments, a steatosis grade of 5-33% comprises 1 point in the NAFLD activity score. In some embodiments, a steatosis grade of 34-66% comprises 2 points in the NAFLD activity score. In some embodiments, a steatosis grade of >66% comprises 3 points in the NAFLD activity score. In some embodiments, the NAFLD activity score comprises a lobular inflammation grade. In some embodiments, the lobular inflammation grade comprises an assessment of inflammatory foci. In some embodiments, a lobular inflammation grade comprising 0 foci comprises 0 points in the NAFLD activity score. In some embodiments, a lobular inflammation grade comprising 1 focus per a field (such as a 20× field or a 200× field) comprises 1 point in the NAFLD activity score. In some embodiments, a lobular inflammation grade comprising 2-4 foci per field comprises 2 points in the NAFLD activity score. In some embodiments, a lobular inflammation grade comprising >4 foci per field comprises 3 points in the NAFLD activity score. In some embodiments, the NAFLD activity score comprises a liver cell injury grade such as an amount of ballooning cells. In some embodiments, a liver cell injury comprising no ballooning cells comprises 0 points in the NAFLD activity score. In some embodiments, a liver cell injury comprising some new balloon cells comprises 1 point in the NAFLD activity score. In some embodiments, a liver cell injury comprising many ballooning cells or prominent ballooning comprises 2 points in the NAFLD activity score. In some embodiments, the NAFLD activity score is obtained invasively, based on histology, and/or in a liver biopsy.

In some embodiments, the composition reduces the NAFLD activity score relative to the baseline NAFLD activity score. In some embodiments, the reduced NAFLD activity score is measured in a second liver sample obtained from the subject after administering the composition to the subject. In some embodiments, the NAFLD activity score is decreased by 1 relative to the baseline NAFLD activity score. In some embodiments, the NAFLD activity score is decreased by 2 relative to the baseline NAFLD activity score. In some embodiments, the NAFLD activity score is decreased by 3 relative to the baseline NAFLD activity score. In some embodiments, the NAFLD activity score is decreased by 4 relative to the baseline NAFLD activity score. In some embodiments, the NAFLD activity score is decreased by 5 relative to the baseline NAFLD activity score. In some embodiments, the NAFLD activity score is decreased by 6 relative to the baseline NAFLD activity score. In some embodiments, the NAFLD activity score is decreased by 7 relative to the baseline NAFLD activity score. In some embodiments, the NAFLD activity score is decreased by 8 relative to the baseline NAFLD activity score. In some embodiments, the NAFLD activity score is decreased by 1 or more, relative to the baseline NAFLD activity score. In some embodiments, the NAFLD activity score is decreased by 2 or more, 3 or more, 4 or more, 5 or more, 6 or more, 7 or more, or 8 or more, relative to the baseline NAFLD activity score. In some embodiments, the NAFLD activity score is decreased by no more than 1, no more than 2, no more than 3, no more than 4, no more than 5, no more than 6, no more than 7, or no more than 8, relative to the baseline NAFLD activity score. In some embodiments, the NAFLD activity score is decreased by 1, 2, 3, 4, 5, 6, 7, or 8, or by a range defined by any of the two aforementioned numbers.

In some embodiments, the measurement is a gamma-glutamyl transferase (GGT) measurement. In some embodiments, the GGT measurement is a GGT concentration. In some embodiments, the GGT measurement is a blood GGT measurement. In some embodiments, the GGT measurement is obtained by an assay such as an immunoassay, a colorimetric assay, a chromatography assay, or a fluorescence assay.

In some embodiments, the composition reduces the GGT measurement relative to the baseline GGT measurement. In some embodiments, the reduced GGT is measured in a second blood sample, plasma sample, or serum sample obtained from the subject after administering the composition to the subject. In some embodiments, the GGT measurement is decreased by about 2.5% or more, about 5% or more, or about 7.5% or more, relative to the baseline GGT measurement. In some embodiments, the GGT measurement is decreased by about 10% or more, relative to the baseline GGT measurement. In some embodiments, the GGT measurement is decreased by about 20% or more, about 30% or more, about 40% or more, about 50% or more, about 60% or more, about 70% or more, about 80% or more, about 90% or more, relative to the baseline GGT measurement. In some embodiments, the GGT measurement is decreased by no more than about 2.5%, no more than about 5%, or no more than about 7.5%, relative to the baseline GGT measurement. In some embodiments, the GGT measurement is decreased by no more than about 10%, relative to the baseline GGT measurement. In some embodiments, the GGT measurement is decreased by no more than about 20%, no more than about 30%, no more than about 40%, no more than about 50%, no more than about 60%, no more than about 70%, no more than about 80%, no more than about 90%, or no more than about 100% relative to the baseline GGT measurement. In some embodiments, the GGT measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the measurement is a hair count. In some embodiments, the hair count is a total hair count. The total hair count may include a vellus hair count and a non-vellus hair count. In some embodiments, the hair count is a vellus hair count. In some embodiments, the hair count is a non-vellus hair count. In some embodiments, the hair count is determined in an area of skin. In some embodiments, the hair count is normalized based on the area of skin. In some embodiments, the hair count is assessed using photography. In some embodiments, the hair count is assessed by phototrichogram. In some embodiments, the hair count is assessed by a macrophotography analysis.

In some embodiments, the composition increases the hair count relative to the baseline hair count. In some embodiments, the increase is measured in the subject after administering the composition to the subject. In some embodiments, the increase is measured directly on the subject after administering the composition to the subject. In some embodiments, the hair count is increased by about 2.5% or more, about 50% or more, or about 7.5% or more, relative to the baseline hair count. In some embodiments, the hair count is increased by about 10% or more, relative to the baseline hair count. In some embodiments, the hair count is increased by about 20% or more, about 30% or more, about 40% or more, about 50% or more, about 60% or more, about 70% or more, about 80% or more, about 90% or more, relative to the baseline hair count. In some embodiments, the hair count is increased by about 100% or more, increased by about 250% or more, increased by about 500% or more, increased by about 750% or more, or increased by about 1000% or more, relative to the baseline hair count. In some embodiments, the hair count is increased by no more than about 2.5%, no more than about 5%, or no more than about 7.5%, relative to the baseline hair count. In some embodiments, the hair count is increased by no more than about 10%, relative to the baseline hair count. In some embodiments, the hair count is increased by no more than about 20%, no more than about 30%, no more than about 40%, no more than about 50%, no more than about 60%, no more than about 70%, no more than about 80%, no more than about 90%, or no more than about 100% relative to the baseline hair count. In some embodiments, the hair count is increased by no more than about 100%, increased by no more than about 250%, increased by no more than about 500%, increased by no more than about 750%, or increased by no more than about 1000%, relative to the baseline hair count. In some embodiments, the hair count is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a hair thickness measurement. In some embodiments, the hair thickness measurement is determined in an area of skin. In some embodiments, the hair thickness measurement comprises a width of an individual hair. In some embodiments, the hair thickness measurement comprises widths of multiple individual hairs. In some embodiments, the hair thickness measurement comprises an average of the widths of the multiple individual hairs. In some embodiments, the hair thickness measurement comprises a median of the widths of the multiple individual hairs. The hair thickness measurement may include a vellus hair thickness measurement. The hair thickness measurement may include a non-vellus hair thickness measurement. In some embodiments, the hair thickness measurement is assessed using photography. In some embodiments, the hair thickness measurement is assessed by phototrichogram. In some embodiments, the hair thickness measurement is assessed by a macrophotography analysis.

In some embodiments, the composition increases the hair thickness measurement relative to the baseline hair thickness measurement. In some embodiments, the increase is measured in a second tissue sample (e.g. a skin sample as described herein) obtained from the subject after administering the composition to the subject. In some embodiments, the increase is measured directly on the subject after administering the composition to the subject. In some embodiments, the hair thickness measurement is increased by about 2.5% or more, about 5% or more, or about 7.5% or more, relative to the baseline hair thickness measurement. In some embodiments, the hair thickness measurement is increased by about 10% or more, relative to the baseline hair thickness measurement. In some embodiments, the hair thickness measurement is increased by about 20% or more, about 30% or more, about 40% or more, about 50% or more, about 60% or more, about 70% or more, about 80% or more, about 90% or more, relative to the baseline hair thickness measurement. In some embodiments, the hair thickness measurement is increased by about 100% or more, increased by about 250% or more, increased by about 500% or more, increased by about 750% or more, or increased by about 1000% or more, relative to the baseline hair thickness measurement. In some embodiments, the hair thickness measurement is increased by no more than about 2.5%, no more than about 5%, or no more than about 7.5%, relative to the baseline hair thickness measurement. In some embodiments, the hair thickness measurement is increased by no more than about 10%, relative to the baseline hair thickness measurement. In some embodiments, the hair thickness measurement is increased by no more than about 20%, no more than about 30%, no more than about 40%, no more than about 50%, no more than about 60%, no more than about 70%, no more than about 80%, no more than about 90%, or no more than about 100% relative to the baseline hair thickness measurement. In some embodiments, the hair thickness measurement is increased by no more than about 100%, increased by no more than about 250%, increased by no more than about 500%, increased by no more than about 750%, or increased by no more than about 1000%, relative to the baseline hair thickness measurement. In some embodiments, the hair thickness measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a hair density measurement. In some embodiments, the hair density measurement is determined in an area of skin. In some embodiments, the hair density measurement comprises a number of hair in the area of skin. In some embodiments, the hair density measurement comprises the number of hair in the area of skin divided by the area of skin. The hair density measurement may include a vellus hair density measurement. The hair density measurement may include a non-vellus hair density measurement. In some embodiments, the hair density measurement is assessed using photography. In some embodiments, the hair density measurement is assessed by phototrichogram. In some embodiments, the hair density measurement is assessed by a macrophotography analysis.

In some embodiments, the composition increases the hair density measurement relative to the baseline hair density measurement. In some embodiments, the increase is measured in a second tissue sample (e.g. a skin sample as described herein) obtained from the subject after administering the composition to the subject. In some embodiments, the increase is measured directly on the subject after administering the composition to the subject. In some embodiments, the hair density measurement is increased by about 2.5% or more, about 5% or more, or about 7.5% or more, relative to the baseline hair density measurement. In some embodiments, the hair density measurement is increased by about 10% or more, relative to the baseline hair density measurement. In some embodiments, the hair density measurement is increased by about 20% or more, about 30% or more, about 40% or more, about 50% or more, about 60% or more, about 70% or more, about 80% or more, about 90% or more, relative to the baseline hair density measurement. In some embodiments, the hair density measurement is increased by about 100% or more, increased by about 250% or more, increased by about 500% or more, increased by about 750% or more, or increased by about 1000% or more, relative to the baseline hair density measurement. In some embodiments, the hair density measurement is increased by no more than about 2.5%, no more than about 5%, or no more than about 7.5%, relative to the baseline hair density measurement. In some embodiments, the hair density measurement is increased by no more than about 10%, relative to the baseline hair density measurement. In some embodiments, the hair density measurement is increased by no more than about 20%, no more than about 30%, no more than about 40%, no more than about 50%, no more than about 60%, no more than about 70%, no more than about 80%, no more than about 90%, or no more than about 100% relative to the baseline hair density measurement. In some embodiments, the hair density measurement is increased by no more than about 100%, increased by no more than about 250%, increased by no more than about 500%, increased by no more than about 750%, or increased by no more than about 1000%, relative to the baseline hair density measurement. In some embodiments, the hair density measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a SOS2 protein measurement. In some embodiments, the SOS2 protein measurement comprises a SOS2 protein level. In some embodiments, the SOS2 protein level is indicated as a mass or percentage of SOS2 protein per sample weight. In some embodiments, the SOS2 protein level is indicated as a mass or percentage of SOS2 protein per sample volume. In some embodiments, the SOS2 protein level is indicated as a mass or percentage of SOS2 protein per total protein within the sample. In some embodiments, the SOS2 protein measurement is a tissue SOS2 protein measurement. Examples of tissue SOS2 protein measurements include a liver SOS2 protein measurement, a kidney SOS2 protein measurement, a eye SOS2 protein measurement, or a adipose tissue SOS2 protein measurement. In some embodiments, the SOS2 protein measurement is a circulating SOS2 protein measurement. In some embodiments, the SOS2 protein measurement is obtained by an assay such as an immunoassay, a colorimetric assay, or a fluorescence assay.

In some embodiments, the composition reduces the SOS2 protein measurement relative to the baseline SOS2 protein measurement. In some embodiments, the reduction is measured in a second sample (e.g. a tissue sample such as liver, kidney, adipose, or eye tissue) obtained from the subject after administering the composition to the subject. In some embodiments, the SOS2 protein measurement is decreased by about 2.5% or more, about 5% or more, or about 7.5% or more, relative to the baseline SOS2 protein measurement. In some embodiments, the SOS2 protein measurement is decreased by about 10% or more, relative to the baseline SOS2 protein measurement. In some embodiments, the SOS2 protein measurement is decreased by about 20% or more, about 30% or more, about 40% or more, about 50% or more, about 60% or more, about 70% or more, about 80% or more, about 90% or more, relative to the baseline SOS2 protein measurement. In some embodiments, the SOS2 protein measurement is decreased by no more than about 2.5%, no more than about 5%, or no more than about 7.5%, relative to the baseline SOS2 protein measurement. In some embodiments, the SOS2 protein measurement is decreased by no more than about 10%, relative to the baseline SOS2 protein measurement. In some embodiments, the SOS2 protein measurement is decreased by no more than about 20%, no more than about 30%, no more than about 40%, no more than about 50%, no more than about 60%, no more than about 70%, no more than about 80%, or no more than about 90% relative to the baseline SOS2 protein measurement. In some embodiments, the SOS2 protein measurement is decreased by 2.5%, 5%, 7.5%, 10%, 20%, 30%, 40%, 50%, 60%, 70%, 80%, or 90%, or by a range defined by any of the two aforementioned percentages.

In some embodiments, the measurement is a SOS2 mRNA measurement. In some embodiments, the SOS2 mRNA measurement comprises a SOS2 mRNA level. In some embodiments, the SOS2 mRNA level is indicated as a mass or percentage of SOS2 mRNA per sample weight. In some embodiments, the SOS2 mRNA level is indicated as a mass or percentage of SOS2 mRNA per sample volume. In some embodiments, the SOS2 mRNA level is indicated as a mass or percentage of SOS2 mRNA per total mRNA within the sample. In some embodiments, the SOS2 mRNA level is indicated as a mass or percentage of SOS2 mRNA per total nucleic acids within the sample. In some embodiments, the SOS2 mRNA level is indicated relative to another mRNA level, such as an mRNA level of a housekeeping gene, within the sample. In some embodiments, the SOS2 mRNA measurement is a tissue SOS2 mRNA measurement. Examples of tissue SOS2 mRNA measurements include a liver SOS2 mRNA measurement, a kidney SOS2 mRNA measurement, a eye SOS2 mRNA measurement, or a adipose tissue SOS2 mRNA measurement. In some embodiments, the SOS2 mRNA measurement is a circulating SOS2 mRNA measurement. In some embodiments, the SOS2 mRNA measurement is obtained by an assay such as a polymerase chain reaction (PCR) assay. In some embodiments, the PCR comprises quantitative PCR (qPCR). In some embodiments, the PCR comprises reverse transcription of the SOS2 mRNA.

In some embodiments, the composition reduces the SOS2 mRNA measurement relative to the baseline SOS2 mRNA measurement. In some embodiments, the reduction is measured in a second sample (e.g. a tissue sample such as liver, kidney, adipose, or eye tissue) obtained from the subject after administering the composition to the subject. The second sample may comprise or consist of hepatocytes. The sample may comprise or consist of podocytes. In some embodiments, the SOS2 mRNA measurement is decreased by about 2.5% or more, about 5% or more, or about 7.5% or more, relative to the baseline SOS2 mRNA measurement. In some embodiments, the SOS2 mRNA measurement is decreased by about 10% or more, relative to the baseline SOS2 mRNA measurement. In some embodiments, the SOS2 mRNA measurement is decreased by about 20% or more, about 30% or more, about 40% or more, about 50% or more, about 60% or more, about 70% or more, about 80% or more, about 90% or more, relative to the baseline SOS2 mRNA measurement. In some embodiments, the SOS2 mRNA measurement is decreased by no more than about 2.5%, no more than about 5%, or no more than about 7.5%, relative to the baseline SOS2 mRNA measurement. In some embodiments, the SOS2 mRNA measurement is decreased by no more than about 10%, relative to the baseline SOS2 mRNA measurement. In some embodiments, the SOS2 mRNA measurement is decreased by no more than about 20%, no more than about 30%, no more than about 40%, no more than about 50%, no more than about 60%, no more than about 70%, no more than about 80%, or no more than about 90% relative to the baseline SOS2 mRNA measurement. In some embodiments, the SOS2 mRNA measurement is decreased by 2.5%, 5%, 7.5%, 10%, 20%, 30%, 40%, 50%, 60%, 70%, 80%, or 90%, or by a range defined by any of the two aforementioned percentages.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

In some embodiments, the term “mRNA” means the presently known mRNA transcript(s) of a targeted gene, and any further transcripts which may be elucidated.

In some embodiments, “dsRNA”, “siRNA”, and “siRNA agent” are used interchangeably as agents that can mediate silencing of a target RNA, e.g., mRNA, e.g., a transcript of a gene that encodes a protein. In some cases, the target RNA is SOS. Such mRNA may also be referred to herein as mRNA to be silenced. Such a gene is also referred to as a target gene. In some cases, the RNA to be silenced is an endogenous gene or a pathogen gene. In addition, RNAs other than mRNA, e.g., tRNAs, and viral RNAs, can also be targeted.

In some embodiments, the phrase “mediates RNAi” refers to the ability to silence, in a sequence specific manner, a target RNA. While not wishing to be bound by theory, it is believed that silencing uses the RNAi machinery or process and a guide RNA, e.g., an siRNA agent.

In some embodiments, “specifically hybridizable” and “complementary” are terms which are used to indicate a sufficient degree of complementarity such that stable and specific binding occurs between a compound described herein and a target RNA molecule.

Specific binding may require a sufficient degree of complementarity to avoid non-specific binding of the oligomeric compound to non-target sequences under conditions in which specific binding is desired, i.e., under physiological conditions in the case of assays or therapeutic treatment, or in the case of in vitro assays, under conditions in which the assays are performed. The non-target sequences may differ by at least 5 nucleotides.

In some embodiments, a dsRNA agent is “sufficiently complementary” to a target RNA, e.g., a target mRNA, such that the dsRNA agent silences production of protein encoded by the target mRNA. In some embodiments, the dsRNA agent is “exactly complementary” to a target RNA, e.g., the target RNA and the dsRNA duplex agent anneal, for example to form a hybrid made exclusively of Watson-Crick base pairs in the region of exact complementarity. A “sufficiently complementary” target RNA can include an internal region (e.g., of at least 10 nucleotides) that is exactly complementary to a target RNA. Moreover, in some embodiments, the dsRNA agent specifically discriminates a single-nucleotide difference. In this case, the dsRNA agent only mediates RNAi if exact complementary is found in the region (e.g., within 7 nucleotides of) the single-nucleotide difference.

In some embodiments, the term “oligonucleotide” refers to a nucleic acid molecule (RNA or DNA) for example of length less than 100, 200, 300, or 400 nucleotides.

In some embodiments, the term “oligonucleotide” refers to an oligomer or polymer of ribonucleic acid (RNA) or deoxyribonucleic acid (DNA) or mimetics thereof. The term “oligonucleotide”, also includes linear or circular oligomers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. Oligonucleotides are capable of specifically binding to a target polynucleotide by way of a regular pattern of monomer-to-monomer interactions, such as Watson-Crick type of base pairing, Hoogsteen or reverse Hoogsteen types of base pairing, or the like.

In some embodiments, the oligonucleotide is “chimeric”, that is, composed of different regions. “Chimeric” oligonucleotides contain two or more chemical regions, for example, DNA region(s), RNA region(s), PNA region(s), etc. Each chemical region is made up of at least one monomer unit, i.e., a nucleotide in the case of an oligonucleotides compound. These oligonucleotides typically comprise at least one region wherein the oligonucleotide is modified in order to exhibit one or more desired properties. The desired properties of the oligonucleotide include, but are not limited, for example, to increased resistance to nuclease degradation, increased cellular uptake, and/or increased binding affinity for the target nucleic acid. Different regions of the oligonucleotide may therefore have different properties. Chimeric oligonucleotides can be formed as mixed structures of two or more oligonucleotides, modified oligonucleotides, oligonucleosides and/or oligonucleotide analogs.

The oligonucleotide can comprise or be composed of regions that can be linked in “register”, that is, when the monomers are linked consecutively, as in native DNA, or linked via spacers. The spacers are intended to constitute a covalent “bridge” between the regions and have, in some cases, a length not exceeding about 100 carbon atoms. The spacers may carry different functionalities, for example, having positive or negative charge, carry special nucleic acid binding properties (intercalators, groove binders, toxins, fluorophores etc.), being lipophilic, inducing special secondary structures like, for example, alanine containing peptides that induce alpha-helices.

In some embodiments, “oligonucleotide specific for” or “oligonucleotide which targets” refers to an oligonucleotide having a sequence (i) capable of forming a stable complex with a portion of the targeted gene, or (ii) capable of forming a stable duplex with a portion of a mRNA transcript of the targeted gene. Stability of the complexes and duplexes can be determined by theoretical calculations and/or in vitro assays.

In some embodiments, the term “target nucleic acid” encompasses DNA, RNA (including pre-mRNA and mRNA) transcribed from such DNA, and also cDNA derived from such RNA, coding, noncoding sequences, sense and antisense polynucleotides. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA that are modulated include, for example, replication and transcription. The functions of RNA that are modulated, include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of an encoded product or oligonucleotides.

RNA interference “RNAi” is mediated by double stranded RNA (dsRNA) molecules that have sequence-specific homology to their “target” nucleic acid sequences. In certain embodiments, the mediators are 5-25 nucleotide “small interfering” RNA duplexes (siRNAs). The siRNAs are derived from the processing of dsRNA by an RNase enzyme known as Dicer. siRNA duplex products are recruited into a multi-protein siRNA complex termed RISC (RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion. Small interfering RNAs can be synthesized and used. Small interfering RNAs for use in the methods herein suitably comprise between about 1 to about 50 nucleotides (nt). In examples of non-limiting embodiments, siRNAs can comprise about 5 to about 40 nt, about 5 to about 30 nt, about 10 to about 30 nt, about 15 to about 25 nt, or about 20-25 nucleotides.

In some embodiments, selection of appropriate oligonucleotides is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of oligonucleotides that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes.

In some embodiments, “enzymatic RNA” is meant as an RNA molecule with enzymatic activity. Enzymatic nucleic acids (ribozymes) act by first binding to a target RNA. Such binding occurs through the target binding portion of an enzymatic nucleic acid which is held in close proximity to an enzymatic portion of the molecule that acts to cleave the target RNA. Thus, the enzymatic nucleic acid first recognizes and then binds a target RNA through base pairing, and once bound to the correct site, acts enzymatically to cut the target RNA.

In some embodiments, “decoy RNA” is meant as an RNA molecule that mimics the natural binding domain for a ligand. The decoy RNA therefore competes with natural binding targets for the binding of a specific ligand. For example, over-expression of HIV trans-activation response (TAR) RNA can act as a “decoy” and efficiently binds HIV tat protein, thereby preventing it from binding to TAR sequences encoded in the HIV RNA. This is meant to be a specific example. Those in the art will recognize that this is but one example, and some embodiments can be readily generated using techniques generally known in the art.

In some embodiments, “monomers” typically indicate monomers linked by phosphodiester bonds or analogs thereof to form oligonucleotides ranging in size from a few monomeric units, e.g., from about 3-4, to about several hundreds of monomeric units. Analogs of phosphodiester linkages include: phosphorothioate, phosphorodithioate, methylphosphornates, phosphoroselenoate, phosphoramidate, and the like, as more fully described below.

In some embodiments, “nucleotide” covers naturally occurring nucleotides as well as non-naturally occurring nucleotides. It should be clear to the person skilled in the art that various nucleotides which previously have been considered “non-naturally occurring” have subsequently been found in nature. Thus, “nucleotides” includes not only the known purine and pyrimidine heterocycles-containing molecules, but also heterocyclic analogues and tautomers thereof. Illustrative examples of other types of nucleotides are molecules containing adenine, guanine, thymine, cytosine, uracil, purine, xanthine, \hat{ }aminopurine, 8-oxo-N6-memyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-memyl-4-triazolopvridin, isocytosine, isoguanin, inosine and the “non-naturally occurring” nucleotides described in Benner et al., U.S. Pat. No. 5,432,272. The term “nucleotide” is intended to cover every and all of these examples as well as analogues and tautomers thereof. Especially interesting nucleotides are those containing adenine, guanine, thymine, cytosine, and uracil, which are considered as the naturally occurring nucleotides in relation to therapeutic and diagnostic application in humans. Nucleotides include the natural 2′-deoxy and 2-hydroxyl sugars, as well as their analogs.

In some embodiments, “analogs” in reference to nucleotides includes synthetic nucleotides having modified base moieties and/or modified sugar moieties. Such analogs include synthetic nucleotides designed to enhance binding properties, e.g., duplex or triplex stability, specificity, or the like.

In some embodiments, “hybridization” means the pairing of at least substantially complementary strands of oligomeric compounds. One mechanism of pairing involves hydrogen bonding, which may be Watson-Crick, Hoogsteen or reversed Hoogsteen hydrogen bonding, between complementary nucleoside or nucleotide bases (nucleotides) of the strands of oligomeric compounds. For example, adenine and thymine are complementary nucleotides which pair through the formation of hydrogen bonds. Hybridization can occur under varying circumstances.

In some embodiments, a compound of the disclosure is “specifically hybridizable” when binding of the compound to the target nucleic acid interferes with the normal function of the target nucleic acid to cause a modulation of function and/or activity, and there is a sufficient degree of complementarity to avoid non-specific binding of the compound to non-target nucleic acid sequences under conditions in which specific binding is desired, i.e., under physiological conditions in the case of in vivo assays or therapeutic treatment, and under conditions in which assays are performed in the case of in vitro assays.

In some embodiments, “stringent hybridization conditions” or “stringent conditions” refers to conditions under which a compound will hybridize to its target sequence, but to a minimal number of other sequences. Stringent conditions are sequence-dependent and will be different in different circumstances and “stringent conditions” under which oligomeric compounds hybridize to a target sequence are determined by the nature and composition of the oligomeric compounds and the assays in which they are being investigated. In some cases, stringent hybridization conditions comprise low concentrations (<0.15M) of salts with inorganic cations such as Na+ or K+ (i.e., low ionic strength), temperature higher than about 20° C. to 25° C. and below the Tm of the oligomeric compound/target sequence complex, and the presence of denaturants such as formamide, dimethylformamide, dimethyl sulfoxide, or the detergent sodium dodecyl sulfate (SDS). For example, the hybridization rate decreases 1.1% for each 1% formamide. An example of a high stringency hybridization condition is 0.1× sodium chloride-sodium citrate buffer (SSC)/0.1% (w/v) SDS at 60° C. for 30 minutes.

In some embodiments, “complementary” refers to the capacity for precise pairing between two nucleotides on one or two oligomeric strands. For example, if a nucleobase at a certain position of a compound is capable of hydrogen bonding with a nucleobase at a certain position of a target nucleic acid, said target nucleic acid being a DNA, RNA, or oligonucleotide molecule, then the position of hydrogen bonding between the oligonucleotide and the target nucleic acid may be considered to be a complementary position. The oligomeric compound and the further DNA, RNA, or oligonucleotide molecule are complementary to each other when a sufficient number of complementary positions in each molecule are occupied by nucleotides which can hydrogen bond with each other. Thus, “specifically hybridizable” and “complementary” are terms which may be used to indicate a sufficient degree of precise pairing or complementarity over a sufficient number of nucleotides such that stable and specific binding occurs between the oligomeric compound and a target nucleic acid.

The sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). In some embodiments, oligomeric compounds disclosed herein comprise at least about 70%, or at least about 75%, or at least about 80%, or at least about 85%, or at least about 90%, or at least about 95%, or at least about 99% sequence complementarity to a target region within the target nucleic acid sequence to which they are targeted. For example, a compound in which 18 of 20 nucleotides of the compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. As such, a compound which is 18 nucleotides in length having 4 (four) noncomplementary nucleotides which are flanked by two regions of complete complementarity with the target nucleic acid would have 77.8% overall complementarity with the target nucleic acid and would thus fall within the scope of the present disclosure. Percent complementarity of a compound with a region of a target nucleic acid can be determined routinely using BLAST programs (basic local alignment search tools) and PowerBLAST programs known in the art. Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman.

In some embodiments, the term “Thermal Melting Point (Tm)” refers to the temperature, under defined ionic strength, pH, and nucleic acid concentration, at which 50% of the oligonucleotides complementary to the target sequence hybridize to the target sequence at equilibrium. Typically, stringent conditions will be those in which the salt concentration is at least about 0.01 to 1.0 M Na ion concentration (or other salts) at pH 7.0 to 8.3 and the temperature is at least about 30° C. for short oligonucleotides (e.g., 10 to 50 nucleotide). Stringent conditions may also be achieved with the addition of destabilizing agents such as formamide.

In some embodiments, “modulation” means either an increase (stimulation) or a decrease (inhibition) in the expression of a gene.

In some embodiments, the term “variant”, when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

Derivative polynucleotides include nucleic acids subjected to chemical modification, for example, replacement of hydrogen by an alkyl, acyl, or amino group. Derivatives, e.g., derivative oligonucleotides, may comprise non-naturally-occurring portions, such as altered sugar moieties or inter-sugar linkages. Exemplary among these are phosphorothioate and other sulfur containing species which are known in the art. Derivative nucleic acids may also contain labels, including radionucleotides, enzymes, fluorescent agents, chemiluminescent agents, chromogenic agents, substrates, co factors, inhibitors, magnetic particles, and the like.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxa-6-azaspiro[3.3]heptane, and 1,1-dioxo-thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

In some embodiments, a “derivative” polypeptide or peptide is one that is modified, for example, by glycosylation, pegylation, phosphorylation, sulfation, reduction/alkylation, acylation, chemical coupling, or mild formalin treatment. A derivative may also be modified to contain a detectable label, either directly or indirectly, including, but not limited to, a radioisotope, fluorescent, and enzyme label.

Some embodiments refer to nucleic acid sequence information. In some embodiments, any uracil (U) may be interchanged with any thymine (T), and vice versa. For example, in an siRNA with a nucleic acid sequence comprising one or more Us, in some embodiments any of the Us may be replaced with Ts. Similarly, in an siRNA with a nucleic acid sequence comprising one or more Ts, in some embodiments any of the Ts may be replaced with Us. In some embodiments, an oligonucleotide such as an siRNA disclosed herein comprises or consists of RNA. In some embodiments, the oligonucleotide may comprise or consist of DNA. To any extent that the sequence listing contradicts the disclosure in the specification, the specification takes precedent.

Some aspects include sequences with nucleotide modifications or modified internucleoside linkages. Generally, and unless otherwise specified, Nf (e.g. Af, Cf, Gf, Tf, or Uf) refers to a 2′ fluoro-modified nucleoside, dN (e.g. dA, dC, dG, dT, or dU) refers to a 2′ deoxy nucleoside, n (e.g. a, c, g, t, or u) refers to a 2′ O-methyl modified nucleoside, and “s” refers to a phosphorothioate linkage.

A pyrimidine may include cytosine (C), thymine (T), or uracil (U). A pyrimidine may include C or U. A pyrimidine may include C or T. A reference to a pyrimidine may include a nucleoside or nucleotide comprising the pyrimidine. A purine may include guanine (G) or adenine (A). A reference to a purine may include a nucleoside or nucleotide comprising a purine.

The section headings used herein are for organizational purposes only and are not to be construed as limiting the subject matter described.

### VI. EXAMPLES

### Example 1: Nonsynonymous Variants in the SOS2 Gene Demonstrate Protective Associations Across a Spectrum of Diseases and Traits

Applicant evaluated two missense variants (Table 5) in approximately 452,000 individuals with genotype data from the UK Biobank cohort for associations with a variety of traits. The variants are rs72681869, a low frequency (EAF=0.01) missense variant (Pro191Arg; P191R) located in the N-terminal histone-fold domain of SOS2, and rs72681869 is a rare (EAF=0.001) missense variant (Leu183Phe; L183F) also located in the N-terminal histone-fold domain. The applicants hypothesize that individually these variants result in a decrease in the abundance and/or activity of the SOS2 gene product, and that it is this loss of function (LoF) that leads to the observed genetic associations. Stepwise conditional analyses in multiple traits, as well as direct evaluation of linkage disequilibrium, confirmed that these are independent variants. Therefore, these two variants were combined and tested in a gene burden test to increase statistical power and collectively represent inhibition of SOS2.

TABLE 5
SOS2 genetic variants/instruments utilized in this study

| | | | Proposed | |
|---|---|---|---|---|
| | | | Effect on | Effect |
| | | | Gene/Protein | Allele |
| | | | Function or | Frequency |
| Variant | Gene | Consequence | Expression | (EAF) |
| rs72681869 | SOS2 | missense (Pro191Arg; | ↓ | 0.01 |
| | | P191R) | | |
| rs137961578 | SOS2 | missense (Leu183Phe; | ↓ | 0.001 |
| | | L183F) | | |
| Burden | SOS2 | P191R and/or L183F | ↓ | 0.01 |

These analyses resulted in identification of pleiotropic associations for the SOS2 missense variants. For example, there were protective associations across a broad range of common cardiometabolic diseases and traits. The SOS2 burden is significantly associated with decreased alanine aminotransferase (ALT) and aspartate aminotransferase (AST), decreased MRI-deriver liver fat percentage, decreased risk of non-alcoholic fatty liver disease (NAFLD) and decreased blood triglycerides, suggesting that SOS2 inhibition may protect against fatty and fibrotic liver disease and hyperlipidemia (Table 6). The SOS2 burden is also associated with decreased risk of type 2 diabetes (T2D), use of diabetes medications, family history of diabetes and decreased hemoglobin A1C, suggesting that SOS2 inhibition may protect against diabetes (Table 7). The SOS2 burden is also associated with decreased risk for obesity, decreased body mass index (BMI), decreased waist and hip circumference and decreased body fat percentage, suggesting that SOS2 inhibition may protect against obesity (Table 8). The SOS2 burden is also associated with decreased risk of hypertension, decreased systolic and diastolic blood pressure (SBP and DBP), decreased mean arterial pressure (MAP) and decreased use of antihypertensive medications, suggesting that SOS2 inhibition may protect against hypertension. (Table 9). The SOS2 burden is also associated with decreased risk of all-cause cerebrovascular disease, ischemic cerebrovascular disease and family history of stroke, suggesting that SOS2 inhibition may protect against cerebrovascular disease (Table 10). The SOS2 burden is also associated with decreased blood urate, decreased risk of gout and decreased use of gout medications, suggesting that SOS2 inhibition may protect against gout and hyperuricemia (Table 11). The SOS2 burden is also associated with increased estimated glomerular filtration rate (eGFR) and decreased blood creatinine, decreased blood urea nitrogen (BUN), decreased risk of chronic kidney disease (CKD), decreased risk of proteinuria and decreased urinary microalbumin, suggesting that SOS2 inhibition may protect against kidney disease (Table 12).

TABLE 6
SOS2 liver associations

| | | | Alanine | | Aspartate | |
|---|---|---|---|---|---|---|
| | | | Aminotransferase | | Aminotransferase | |
| | | | (n = 430,545) | | (n = 429,246) | |
| Variant | Gene | EAF | P value | Beta | P value | Beta |
| Burden | SOS2 | 0.01 | 8.99E−24 | ↓−0.019 | 1.35E−31 | ↓−0.013 |

| | | | MRI Liver Fat % | | Blood Triglycerides | |
|---|---|---|---|---|---|---|
| | | | (n = 14,875) | | (n = 430,833) | |
| Variant | Gene | EAF | P value | Beta | P value | Beta |
| Burden | SOS2 | 0.01 | 0.002 | ↓−0.163 | 1.95E−07 | ↓−0.011 |

Non-Alcoholic Fatty
Liver Disease
(n = 7,792)

| Variant | Gene | EAF | P value | OR |
|---|---|---|---|---|
| Burden | SOS2 | 0.01 | 0.002 | ↓0.838 |

TABLE 7
SOS2 diabetes associations

| | | | Type 2 Diabetes mellitus | | Rx for Diabetes | |
|---|---|---|---|---|---|---|
| | | | (n = 37,012) | | (n = 29,596) | |
| Variant | Gene | EAF | P value | OR | P value | OR |
| Burden | SOS2 | 0.01 | 2.61E−07 | ↓0.838 | 2.89E−05 | ↓0.842 |

| | | | Family History of Diabetes | | Hemoglobin A1C | |
|---|---|---|---|---|---|---|
| | | | (n = 92,301) | | (n = 410,975) | |
| Variant | Gene | EAF | P value | OR | P value | Beta |
| Burden | SOS2 | 0.01 | 8.64E−04 | ↓0.921 | 3.63E−12 | ↓−0.003 |

TABLE 8
SOS2 obesity associations

| | | | Obesity | | Body Mass Index | |
|---|---|---|---|---|---|---|
| | | | (n = 51,170) | | (n = 445,825) | |
| Variant | Gene | EAF | P value | OR | P value | Beta |
| Burden | SOS2 | 0.01 | 2.07E−04 | ↓0.868 | 3.13E−08 | ↓−0.050 |

| | | | Waist Circumference | | Hip Circumference | |
|---|---|---|---|---|---|---|
| | | | (n = 449,256) | | (n = 446,083) | |
| Variant | Gene | EAF | P value | Beta | P value | Beta |
| Burden | SOS2 | 0.01 | 8.52E−09 | ↓−0.003 | 3.62E−09 | ↓−0.002 |

Body Fat %
(n = 444,332)

| Variant | Gene | EAF | P value | Beta |
|---|---|---|---|---|
| Burden | SOS2 | 0.01 | 5.57E−12 | ↓−0.007 |

TABLE 9
SOS2 hypertension associations

| | | | Essential | | Systolic Blood | |
|---|---|---|---|---|---|---|
| | | | Hypertension | | Pressure | |
| | | | (n = 177,488) | | (n = 425,193) | |
| Variant | Gene | EAF | P value | OR | P value | Beta |
| Burden | SOS2 | 0.01 | 7.07E−12 | ↓0.831 | 4.82E−18 | ↓−0.006 |

| | | | Diastolic Blood | | Mean Arterial | |
|---|---|---|---|---|---|---|
| | | | Pressure | | Pressure | |
| | | | (n = 425,809) | | (n = 423,798) | |
| Variant | Gene | EAF | P value | Beta | P value | Beta |
| Burden | SOS2 | 0.01 | 8.82E−20 | ↓−0.006 | 8.10E−21 | ↓−1.343 |

Rx for Hypertension
(n = 130,907)

| Variant | Gene | EAF | P value | OR |
|---|---|---|---|---|
| Burden | SOS2 | 0.01 | 6.12E−13 | ↓0.818 |

TABLE 10
cerebrovascular associations

| | | | Cerebrovascular | | Ischemic | | Family History | |
|---|---|---|---|---|---|---|---|---|
| | | | Diseases | | Cerebrovascular | | of Stroke | |
| | | | (n = 29,617) | | Disease (n = 18,115) | | (n = 119,462) | |
| Variant | Gene | EAF | P value | OR | P value | OR | P value | OR |
| Burden | SOS2 | 0.01 | 1.05E−05 | ↓0.846 | 5.05E−04 | ↓0.853 | 0.027 | ↓0.951 |

TABLE 11
SOS2 gout associations

| | | | Blood Urate | | Gout | | Rx for Gout | |
|---|---|---|---|---|---|---|---|---|
| | | | (n = 431,774) | | (n = 24,226) | | (n = 22,408) | |
| Variant | Gene | EAF | P value | Beta | P value | OR | P value | OR |
| Burden | SOS2 | 0.01 | 6.17E−14 | ↓−0.009 | 3.62E−07 | ↓0.767 | 7.58E−09 | ↓0.755 |

TABLE 12
kidney associations

| | | | eGFR | | Creatinine | |
|---|---|---|---|---|---|---|
| | | | (n = 432,345) | | (n = 430,258) | |
| Variant | Gene | EAF | P value | Beta | P value | Beta |
| Burden | SOS2 | 0.01 | 3.94E−12 | ↑0.066 | 3.86E−12 | ↓−0.006 |

| | | | BUN | | Chronic Kidney Disease | |
|---|---|---|---|---|---|---|
| | | | (n = 431,470) | | (n = 23,130) | |
| Variant | Gene | EAF | P value | Beta | P value | OR |
| Burden | SOS2 | 0.01 | 3.27E−04 | ↓−0.004 | 8.57E−05 | ↓0.867 |

| | | | Proteinuria | | Urine Microalbumin | |
|---|---|---|---|---|---|---|
| | | | (n = 2,801) | | (n = 138,748) | |
| Variant | Gene | EAF | P value | OR | P value | Beta |
| Burden | SOS2 | 0.01 | 0.043 | ↓0.836 | 9.29E−03 | ↓−0.045 |

Applicant also identified protective associations across a range of ophthalmic diseases and traits. For example, the SOS2 burden is associated with decreased risk of glaucoma, primary open-angle glaucoma (POAG), decreased use of glaucoma medications, decreased risk of surgery for glaucoma, decreased intraocular pressure and decreased vertical cup-disc ratio, suggesting that SOS2 inhibition may protect against glaucoma and ocular hypertension (Table 13). The SOS2 burden is also associated with decreased risk of macular degeneration, diabetic retinopathy and decreased thickness of the retinal pigment epithelium (RPE), suggesting that SOS2 inhibition may protect against retinal disorders such as macular degeneration and diabetic retinopathy (Table 14).

TABLE 13
SOS2 glaucoma associations

| | | | Glaucoma | | Primary Open-Angle | |
|---|---|---|---|---|---|---|
| | | | (n = 18,181) | | Glaucoma (n = 3,925) | |
| Variant | Gene | EAF | P value | OR | P value | OR |
| Burden | SOS2 | 0.01 | 7.14E−06 | ↓0.824 | 3.19E−07 | ↓0.634 |
| | | | Rx for Glaucoma | | Surgery for Glaucoma | |
| | | | (n = 16,721) | | (n = 1,960) | |
| Variant | Gene | EAF | P value | OR | P value | OR |
| Burden | SOS2 | 0.01 | 7.72E−06 | ↓0.782 | 7.90E−05 | ↓0.475 |

| | | | Intraocular Pressure | | OCT Vertical Cup-Disc | |
|---|---|---|---|---|---|---|
| | | | (n = 108,466) | | Ratio (n = 60,439) | |
| Variant | Gene | EAF | P value | Beta | P value | Beta |
| Burden | SOS2 | 0.01 | 5.83E−09 | ↓−0.113 | 0.001 | ↓−0.015 |

TABLE 14
SOS2 retinal disease associations

| | | | Macular | | Diabetic | | OCT Retinal Pigment | |
|---|---|---|---|---|---|---|---|---|
| | | | Degeneration | | Retinopathy | | Epithelium Thickness | |
| | | | (n = 13,565) | | (n = 8,974) | | (n = 69,955) | |
| Variant | Gene | EAF | P value | OR | P value | OR | P value | Beta |
| Burden | SOS2 | 0.01 | 1.17E−04 | ↓0.827 | 6.34E−04 | ↓0.833 | 0.001 | ↑0.080 |

These protective associations with putative loss of function variants in SOS2 across several related and distinct diseases and traits suggest that inhibition of SOS2 could be therapeutic in these diseases.

### Example 2: SiRNA-Mediated Knockdown of SOS2 in PODO/TERT256 Cell Line

siRNAs will be targeted to SOS2 mRNA that may downregulate levels of SOS2 mRNA leading to a decrease in RAC1 protein activation, when administered to the cultured immortalized human podocyte cell line PODO/TERT256 (Evercyte Cat. No. CHT-033-0256).

On Day 0, the PODO/TERT256 cells will be seeded at 150,000 cells/mL into a Falcon 24-well tissue culture plate (ThermoFisher Cat. No. 353047) at 0.5 mL per well.

On Day 1, the SOS2 siRNA and negative control siRNA master mixes will be prepared. The SOS2 siRNA master mix will contain 350 μL of Opti-MEM (ThermoFisher Cat. No. 4427037-s1288 Lot No. AS02B02D) and 3.5 ul of a mixture of the two SOS2 siRNAs (10 uM stock). The negative control siRNA master mix will contain 350 μL of Opti-MEM and 3.5 ul of negative control siRNA (ThermoFisher Cat. No. 4390843, 10 uM stock). Next, 3 μL of TransIT-X2 (Mirus Cat. No. MIR-6000) is added to each master mix. The mixes are incubated for 15 minutes to allow transfection complexes to form, then 51 ul of the appropriate master mix+TransIT-X2 is added to duplicate wells of PODO/TERT256 cells with a final siRNA concentration of 10 nM.

On Day 3, 48 hours post-transfection, duplicate wells will be lysed using the Cells-to-Ct kit according to the manufacturer's protocol (ThermoFisher Cat. No. 4399002) or protein lysis buffer containing protease and phosphatase inhibitors. For the Cells-to-Ct, cells are washed with 50 ul using cold 1×PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). For the protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. Primary immunoblotting antibodies are: anti-GAPDH, anti-RAC1, anti-GTP-RAC1 (Sigma, MO) and anti-SOS2 (Abcam, Cambridge, UK).

A decrease in SOS2 mRNA expression in the PODO/TERT256 cells is expected after transfection with the SOS2 siRNAs compared to SOS2 mRNA levels in PODO/TERT256 cells transfected with the non-specific control siRNA 48 hours after transfection. There is an expected decrease in the amount of activated RAC1, measured by quantifying the amount of GTP bound RAC1 versus total RAC1 in wells containing PODO/TERT256 cells transfected with the SOS2 siRNAs relative to the amount of GTP bound RAC1 versus total RAC1 in wells containing PODO/TERT256 cells transfected with a non-specific control siRNA 48 hours after transfection. These results are expected to show that the SOS2 siRNAs elicit knockdown of SOS2 mRNA in PODO/TERT256 cells and that the decrease in SOS2 expression is correlated with a decrease in activated RAC1.

### Example 3: ASO-Mediated Knockdown of SOS2 in PODO/TERT256 Cell Line

ASOs will be targeted to SOS2 mRNA that may downregulate levels of SOS2 mRNA leading to a decrease in RAC1 protein activation, when administered to the cultured immortalized human podocyte cell line PODO/TERT256 (Evercyte Cat. No. CHT-033-0256).

On Day 0, the PODO/TERT256 cells will be seeded at 150,000 cells/mL into a Falcon 24-well tissue culture plate (ThermoFisher Cat. No. 353047) at 0.5 mL per well.

On Day 1, the SOS2 ASO and negative control ASO master mixes will be prepared. The SOS2 ASO master mix will contain 350 uL of Opti-MEM (ThermoFisher Cat. No. 4427037-s1288 Lot No. AS02B02D) and 3.5 ul of a mixture of the two SOS2 ASOs (10 uM stock). The negative control ASO master mix will contain 350 uL of Opti-MEM and 3.5 ul of negative control ASO (ThermoFisher Cat. No. 4390843, 10 uM stock). Next, 3 uL of TransIT-X2 (Mirus Cat. No. MIR-6000) is added to each master mix. The mixes are incubated for 15 minutes to allow transfection complexes to form, then 51 ul of the appropriate master mix+TransIT-X2 is added to duplicate wells of PODO/TERT256 cells with a final ASO concentration of 10 nM.

On Day 3, 48 hours post transfection, duplicate wells will be lysed using the Cells-to-Ct kit according to the manufacturer's protocol (ThermoFisher Cat. No. 4399002) or protein lysis buffer containing protease and phosphatase inhibitors. For the Cells-to-Ct, cells are washed with 50 ul using cold 1×PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). For protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. Primary immunoblotting antibodies are: anti-GAPDH, anti-RAC1, anti-GTP-RAC1 (Sigma, MO) and anti-SOS2 (Abcam, Cambridge, UK).

A decrease in SOS2 mRNA expression in the PODO/TERT256 cells is expected after transfection with the SOS2 ASOs compared to SOS2 mRNA levels in PODO/TERT256 cells transfected with the non-specific control ASO 48 hours after transfection. There is an expected decrease in the amount of activated RAC1, measured by quantifying the amount of GTP bound RAC1 versus total RAC1 in wells containing PODO/TERT256 cells transfected with the SOS2 ASOs relative to the amount of GTP bound RAC1 versus total RAC1 in wells containing PODO/TERT256 cells transfected with a non-specific control ASO 48 hours after transfection. These results are expected to show that the SOS2 ASOs elicit knockdown of SOS2 mRNA in PODO/TERT256 cells and that the decrease in SOS2 expression is correlated with a decrease in activated RAC1.

### Example 4: SiRNA-Mediated Knockdown of SOS2 in HepG2 Cell Line

siRNAs targeted to the SOS2 mRNA that downregulate levels of SOS2 mRNA leading to a decrease in RAC1 protein activation, when administered to the cultured human hepatocyte cell line, HepG2.

On Day 0, the HepG2 cells are seeded at 150,000 cells/mL into a Falcon 24-well tissue culture plate (ThermoFisher Cat. No. 353047) at 0.5 mL per well.

On Day 1, the SOS2 siRNA and negative control siRNA master mixes are prepared. The SOS2 siRNA master mix contains 350 uL of Opti-MEM (ThermoFisher Cat. No. 4427037-s1288 Lot No. AS02B02D) and 3.5 ul of a mixture of two SOS2 siRNAs (10 uM stock). The negative control siRNA master mix contains 350 uL of Opti-MEM and 3.5 ul of negative control siRNA (ThermoFisher Cat. No. 4390843, 10 uM stock). Next, 3 uL of TransIT-X2 (Mirus Cat. No. MIR-6000) is added to each master mix. The mixes are incubated for 15 minutes to allow transfection complexes to form, then 51 ul of the appropriate master mix+TransIT-X2 is added to duplicate wells of HepG2 cells with a final siRNA concentration of 10 nM.

On Day 4, 72 hours post transfection, the cells are lysed using the Cells-to-Ct kit according to the manufacturer's protocol (ThermoFisher Cat. No. 4399002). For the Cells-to-Ct protocol, cells are washed with 50 ul using cold 1×PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). For the protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. Primary immunoblotting antibodies are: anti-GAPDH, anti-RAC1, anti-GTP-RAC1 (Sigma, MO) and anti-SOS2 (Abcam, Cambridge, UK).

A decrease in SOS2 mRNA expression in the HepG2 cells is expected after transfection with the SOS2 siRNAs compared to SOS2 mRNA levels in HepG2 cells transfected with the non-specific control siRNA 72 hours after transfection. There is an expected decrease in the amount of activated RAC1, measured by quantifying the amount of GTP bound RAC1 versus total RAC1 in HepG2 cells transfected with the SOS2 siRNAs relative to the amount of GTP bound RAC1 versus total RAC1 in HepG2 cells transfected with a non-specific control siRNA 72 hours after transfection. These results show that the SOS2 siRNAs elicit knockdown of SOS2 mRNA in HepG2 cells and that the decrease in SOS2 expression is correlated with a decrease in activated RAC1.

### Example 5: ASO-Mediated Knockdown of SOS2 in HepG2 Cell Line

ASOs targeted to the SOS2 mRNA that downregulate levels of SOS2 mRNA leading to a decrease in RAC1 protein activation, when administered to the cultured human hepatocyte cell line, HepG2.

On Day 0, the HepG2 cells are seeded at 150,000 cells/mL into a Falcon 24-well tissue culture plate (ThermoFisher Cat. No. 353047) at 0.5 mL per well.

On Day 1, the SOS2 ASO and negative control ASO master mixes are prepared. The SOS2 siRNA master mix contains 350 uL of Opti-MEM (ThermoFisher Cat. No. 4427037-s1288 Lot No. AS02B02D) and 3.5 ul of a mixture of two SOS2 ASOs (10 uM stock). The negative control siRNA master mix contains 350 uL of Opti-MEM and 3.5 ul of negative control ASO (ThermoFisher, 10 uM stock). Next, 3 uL of TransIT-X2 (Mirus Cat. No. MIR-6000) is added to each master mix. The mixes are incubated for 15 minutes to allow transfection complexes to form, then 51 ul of the appropriate master mix+TransIT-X2 is added to duplicate wells of HepG2 cells with a final ASO concentration of 10 nM.

On Day 4, 72 hours post transfection, the cells are lysed using the Cells-to-Ct kit according to the manufacturer's protocol (ThermoFisher Cat. No. 4399002). For the Cells-to-Ct protocol, cells are washed with 50 ul using cold 1×PBS and lysed by adding 49.5 ul of Lysis Solution and 0.5 ul DNase I per well and pipetting up and down 5 times and incubating for 5 minutes at room temperature. The Stop Solution (5 ul/well) is added to each well and mixed by pipetting up and down five times and incubating at room temperature for 2 minutes. The reverse transcriptase reaction is performed using 22.5 ul of the lysate according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). For the protein quantification, equivalent quantities (30-50 μg) of protein are separated by 10% SDS polyacrylamide gels and transferred to polyvinylidene fluoride membranes. Membranes are blocked with 5% nonfat milk and incubated overnight with the appropriate primary antibody at dilutions specified by the manufacturer. Next, the membranes are washed three times in TBST and incubated with the corresponding horseradish peroxidase conjugated secondary antibody at 1:5,000 dilution for 1 hr. Bound secondary antibody is detected using an enhanced chemiluminescence system. Primary immunoblotting antibodies are: anti-GAPDH, anti-RAC1, anti-GTP-RAC1 (Sigma, MO) and anti-SOS2 (Abcam, Cambridge, UK).

A decrease in SOS2 mRNA expression in the HepG2 cells is expected after transfection with the SOS2 ASOs compared to SOS2 mRNA levels in HepG2 cells transfected with the non-specific control ASO 72 hours after transfection. There is an expected decrease in the amount of activated RAC1, measured by quantifying the amount of GTP bound RAC1 versus total RAC1 in HepG2 cells transfected with the SOS2 ASOs relative to the amount of GTP bound RAC1 versus total RAC1 in HepG2 cells transfected with a non-specific control ASO 72 hours after transfection. These results show that the SOS2 ASOs elicit knockdown of SOS2 mRNA in HepG2 cells and that the decrease in SOS2 expression is correlated with a decrease in activated RAC1.

### Example 6: Inhibition of SOS2 in a Mouse Model for Chronic Kidney Disease Using SOS2 siRNAs or ASOs

In this experiment, a mouse model of chronic kidney disease (CKD) will be used to evaluate the effect of siRNA or ASO inhibition of SOS2. The chronic kidney disease model involves temporarily occluding blood flow to both kidneys in 12-week-old C57BL mice. Kidney function is monitored by measuring serum creatinine, blood urea nitrogen, and urine albumin.

Mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group will include eight mice (4 males, 4 females).

Administration of siRNA or ASO will be achieved with a 100 ul subcutaneous injection of siRNA or ASO resuspended in PBS at concentration of 10 uM. On Study Day 0, Group 1 are be injected subcutaneously with non-targeting control siRNA, Group 2 mice are injected subcutaneously with non-targeting control ASO, Group 3 mice are injected subcutaneously with siRNA1 targeting mouse SOS2, Group 4 mice are injected subcutaneously with ASO1 targeting mouse SOS2, and Group 5 mice are injected subcutaneously with vehicle. Every 7 days after the first injection animals from each group are dosed for a total of 2 injections. Blood and urine samples are taken every other day and urine collected for a 24 hr period weekly; serum creatinine, blood urea nitrogen, blood uric acid and urine albumin are measured.

14 days after the ischemia reperfusion surgery to induce the chronic kidney disease like phenotype, the mice will be sacrificed by cervical dislocation following an intraperitoneal injection of 0.3 ml Nembutal (5 mg/ml) (Sigma Cat. No. 1507002). Final blood and urine samples are collected, and kidneys are removed and placed in RNAlater for mRNA isolation.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). A decrease in SOS2 mRNA expression in the kidney tissue from mice dosed with the SOS2 siRNA1 or ASO1 is expected compared to SOS2 mRNA levels in the kidney tissue from mice dosed with the non-specific controls. There is an expected decrease in serum creatinine, urea nitrogen, albumin, and urate in mice that receive the SOS2 siRNA or ASO compared to the serum creatinine, urea nitrogen, albumin, and urate in mice that receive the non-specific control. These results are expected to show that the SOS2 siRNA or ASO elicit knockdown of SOS2 mRNA in kidney tissue and that the decrease in SOS2 expression is correlated with a decrease in serum creatinine, blood urea nitrogen, and urine albumin.

### Example 7: Inhibition of SOS2 in a Mouse Model for Hyperuricemia-Induced Chronic Kidney Disease Using SOS2 siRNAs or ASOs

In this experiment, a mouse model of hyperuricemia-induced chronic kidney disease will be used to evaluate the effect of siRNA or ASO inhibition of SOS2. The hyperuricemia-induced chronic kidney disease model is induced by gavage of a mixture of adenine (160 mg/kg/d) and potassium oxonate (2400 mg/kg/d) (Sigma-Aldrich, St. Louis, MO) dissolved in 200 μL double distilled water daily consistently for 3 weeks in C57BL/6 mice (8-10 weeks of age weighing 25-27 g). Kidney function is monitored by measuring serum creatinine, blood urea nitrogen, blood uric acid and urine albumin.

3 hours after model induction, mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group contains eight mice (4 males, 4 females).

21 days after the model induction, the mice will be sacrificed by cervical dislocation following an intraperitoneal injection of 0.3 ml Nembutal (5 mg/ml) (Sigma Cat. No. 1507002). Final blood and urine samples are collected, and kidneys are removed and placed in RNAlater for mRNA isolation.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR was performed in triplicate using TaqMan Gene Expression Assays (Applied Biosystems FAM/SOS2 using a BioRad CFX96 Cat. No. 1855195). A decrease in SOS2 mRNA expression in the kidney tissue from mice dosed with the SOS2 siRNA1 or ASO1 is expected compared to SOS2 mRNA levels in the kidney tissue from mice dosed with the non-specific controls. There is an expected decrease in serum creatinine, blood urea nitrogen, blood uric acid and urine albumin in mice that receive the SOS2 siRNA or ASO compared to the serum creatinine, blood urea nitrogen, blood uric acid and urine albumin in mice that receive the non-specific control. These results are expected to show that the SOS2 siRNA or ASO elicit knockdown of SOS2 mRNA in kidney tissue and that the decrease in SOS2 expression is correlated with a decrease in serum creatinine, blood urea nitrogen, blood uric acid and urine albumin.

### Example 8: Inhibition of SOS2 in a Mouse Model of Glaucoma Using SOS2 siRNAs or ASOs

In this experiment, a mouse model of glaucoma will be used to evaluate the effect of siRNA or ASO inhibition of SOS2. The glaucoma model involves injection of adenovirus expressing TGF-B into the vitreous fluid of the eyes of C57BL mice. Glaucoma like conditions are monitored by measuring intra-ocular pressure (IOP) of the eyes of mice treated with the adenovirus expressing TGF-B.

Mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group contains eight mice (4 males, 4 females).

Administration of siRNA or ASO will be achieved with a 2 ul intravitreal injection of siRNA or ASO resuspended in PBS at concentration of 10 uM. On Study Day 0, Group 1 mice are injected intravitreally with non-targeting control siRNA, Group 2 mice are injected intravitreally with non-targeting control ASO, Group 3 mice are injected intravitreally with siRNA1 targeting mouse SOS2, Group 4 mice are injected intravitreally with ASO1 targeting mouse SOS2, and Group 5 mice are injected intravitreally with vehicle. Every 7 days after the first injection animals from each group will be dosed for a total of 2 injections. IOP measurement are taken every other day.

14 days after the first injection of siRNA or ASO, the mice will be sacrificed by cervical dislocation following an intraperitoneal injection of 0.3 ml Nembutal (5 mg/ml) (Sigma Cat. No. 1507002). Eyes are removed and placed in RNAlater for mRNA isolation.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR was performed in triplicate using TaqMan Gene Expression Assays (Applied Biosystems FAM/SOS2 using a BioRad CFX96 Cat. No. 1855195). A decrease in SOS2 mRNA expression in the eye tissue from mice dosed with the SOS2 siRNA1 or ASO1 is expected compared to SOS2 mRNA levels in the eye tissue from mice dosed with the non-specific controls. There is an expected decrease in IOP in mice that receive the SOS2 siRNA or ASO compared to the IOP in mice that receive the non-specific control. These results are expected to show that the SOS2 siRNA or ASO elicit knockdown of SOS2 mRNA in eye tissue and that the decrease in SOS2 expression is correlated with a decrease in IOP.

### Example 9: Inhibition of SOS2 in a Mouse Model for NASH/NAFLD Using SOS2 siRNAs or ASOs

In this experiment, a mouse model of NASH/NAFLD will be used to evaluate the effect of siRNA or ASO inhibition of SOS2. The NASH/NAFLD disease model mice are fed a high fructose, high fat, high cholesterol diet in ob/ob mice for 12 weeks prior to treatment. Liver function, glucose homeostasis and obesity are monitored by measuring blood glucose, ALT, insulin, blood lipids and body weight.

Mice will be divided into five groups: Group 1—a group treated with non-targeting control siRNA, Group 2—a group treated with non-targeting control ASO, Group 3—a group treated with SOS2 siRNA1, Group 4—a group treated with SOS2 ASO1, Group 5—a group treated with vehicle. Each group contains eight mice (4 males, 4 females).

Administration of siRNA or ASO will be achieved with a 100 ul subcutaneous injection of siRNA or ASO resuspended in PBS at concentration of 10 uM. On Study Day 0, Group 1 mice are injected subcutaneously with non-targeting control siRNA, Group 2 mice are injected subcutaneously with non-targeting control ASO, Group 3 mice are injected subcutaneously with siRNA1 targeting mouse SOS2, Group 4 mice are injected subcutaneously with ASO1 targeting mouse SOS2, and Group 5 mice are injected subcutaneously with vehicle. Every 7 days after the first injection animals from each group will be dosed for a total of 3 injections. Body weights are measured, and blood samples are taken twice per week; blood glucose, ALT, insulin, and lipids are measured.

6 weeks after the treatment, the mice will be sacrificed by cervical dislocation following an intraperitoneal injection of 0.3 ml Nembutal (5 mg/ml) (Sigma Cat. No. 1507002). Final blood samples are collected, and livers and adipose tissue are removed, and a section placed in RNAlater for mRNA isolation or neutral buffered formalin for histopathology.

mRNA will be isolated from tissue placed in RNAlater solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM/SOS2 using a BioRad CFX96 Cat. No. 1855195). A decrease in SOS2 mRNA expression in the liver and adipose tissue from mice dosed with the SOS2 siRNA1 or ASO1 is expected compared to SOS2 mRNA levels in the liver and adipose tissue from mice dosed with the non-specific controls. There is an expected decrease in body weight, blood glucose, ALT, insulin, and lipids in mice that receive the SOS2 siRNA or ASO compared to the body weight, blood glucose, ALT, insulin, and lipids in mice that receive the non-specific control. These results are expected to show that the SOS2 siRNA or ASO elicit knockdown of SOS2 mRNA in liver tissue and that the decrease in SOS2 expression is correlated with a decrease in body weight, blood glucose, ALT, insulin, and lipids.

Additionally, liver sections placed in the neutral buffered formalin will be embedded into paraffin and sectioned onto slide. The slide from both treated and non-treated mice are then assessed by histopathology using semi-quantitative scoring system (H&E/Picrosirius Red). There is an expected decrease in histopathology fatty liver and fibrosis scoring in mice that receive the SOS2 siRNA or ASO compared to the histopathology fatty liver and fibrosis scoring in mice that receive the non-specific control. These are expected to results show that the SOS2 siRNA or ASO elicit knockdown of SOS2 mRNA in liver tissue and that the decrease in SOS2 expression is correlated with a decrease in histopathology fatty liver and fibrosis scoring.

### Example 10: Bioinformatic Selection of Sequences in Order to Identify Therapeutic siRNAs to Downmodulate Expression of the SOS2 mRNA

Screening sets were defined based on bioinformatic analysis. Therapeutic siRNAs were designed to target human SOS2. Predicted specificity in human, rhesus monkey, cynomolgus monkey, mouse, rat, rabbit, and dog was determined for sense (S) and antisense (AS) strands. These were assigned a “specificity score” which considers the likelihood of unintended downregulation of any other transcript by full or partial complementarity of an siRNA strand (up to 2 mismatches within positions 2-18) as well as the number and positions of mismatches. Thus, off-target(s) transcripts for antisense and sense strands of each siRNA were identified. As identified, siRNAs with high specificity and a low number of predicted off-targets provided a benefit of increased targeting specificity.

In addition to selecting siRNA sequences with high sequence specificity to SOS2 mRNA, siRNA sequences within the seed region were analyzed for similarity to seed regions of known miRNAs. siRNAs can function in a miRNA like manner via base-pairing with complementary sequences within the 3′-UTR of mRNA molecules. The complementarity typically encompasses the 5′-bases at positions 2-7 of the miRNA (seed region). To circumvent siRNAs to act via functional miRNA binding sites, siRNA strands containing natural miRNA seed regions can be avoided. Seed regions identified in miRNAs from human, mouse, rat, rhesus monkey, dog, rabbit, and pig are referred to as “conserved”. Combining the “specificity score” with miRNA seed analysis yielded a “specificity category”. This is divided into categories 1-4, with 1 having the highest specificity and 4 having the lowest specificity. Each strand of the siRNA is assigned to a specificity category.

Analysis of the Genome Aggregation Database (gnomAD) to identify siRNAs targeting regions with known SNPs was also carried out to identify siRNAs that may be non-functional in individuals containing the SNP. Information regarding the positions of SNPs within the target sequence as well as minor allele frequency (MAF) in case data was obtained in this analysis.

Initial analysis of the relevant SOS2 mRNA sequence revealed few sequences that fulfil the specificity parameters and at the same time target SOS2 mRNA in all the analyzed relevant species. Therefore, independent screening subsets were designed for the therapeutic siRNAs.

The siRNAs in these subsets recognized at least the human SOS2 sequences. Therefore, the siRNAs in these subsets can be used to target human SOS2 in a therapeutic setting.

The number of siRNA sequences derived from human SOS2 mRNA (ENST00000216373.10; SEQ ID NO: 11253) without consideration of specificity or species cross-reactivity was 5490 (sense and antisense strand sequences included in SEQ ID NOS: 1-5490 and 5491-10980, respectively)

Prioritizing sequences for target specificity, absence of miRNA seed region sequences and SNPs as described above yields subset A. Subset A contains 1126 siRNAs whose base sequences are shown in Table 15.

TABLE 15
Subset A

| | Sense | Antisense |
|---|---|---|
| siRNA Name | SEQ ID NO: | SEQ ID NO: |
| siRNA 47 | 47 | 5537 |
| siRNA 52 | 52 | 5542 |
| siRNA 53 | 53 | 5543 |
| siRNA 72 | 72 | 5562 |
| siRNA 105 | 105 | 5595 |
| siRNA 106 | 106 | 5596 |
| siRNA 108 | 108 | 5598 |
| siRNA 115 | 115 | 5605 |
| siRNA 129 | 129 | 5619 |
| siRNA 158 | 158 | 5648 |
| siRNA 159 | 159 | 5649 |
| siRNA 160 | 160 | 5650 |
| siRNA 164 | 164 | 5654 |
| siRNA 165 | 165 | 5655 |
| siRNA 200 | 200 | 5690 |
| siRNA 224 | 224 | 5714 |
| siRNA 233 | 233 | 5723 |
| siRNA 234 | 234 | 5724 |
| siRNA 236 | 236 | 5726 |
| siRNA 258 | 258 | 5748 |
| siRNA 266 | 266 | 5756 |
| siRNA 275 | 275 | 5765 |
| siRNA 281 | 281 | 5771 |
| siRNA 282 | 282 | 5772 |
| siRNA 301 | 301 | 5791 |
| siRNA 303 | 303 | 5793 |
| siRNA 305 | 305 | 5795 |
| siRNA 308 | 308 | 5798 |
| siRNA 310 | 310 | 5800 |
| siRNA 313 | 313 | 5803 |
| siRNA 315 | 315 | 5805 |
| siRNA 317 | 317 | 5807 |
| siRNA 323 | 323 | 5813 |
| siRNA 326 | 326 | 5816 |
| siRNA 331 | 331 | 5821 |
| siRNA 332 | 332 | 5822 |
| siRNA 336 | 336 | 5826 |
| siRNA 338 | 338 | 5828 |
| siRNA 341 | 341 | 5831 |
| siRNA 342 | 342 | 5832 |
| siRNA 347 | 347 | 5837 |
| siRNA 350 | 350 | 5840 |
| siRNA 351 | 351 | 5841 |
| siRNA 352 | 352 | 5842 |
| siRNA 353 | 353 | 5843 |
| siRNA 354 | 354 | 5844 |
| siRNA 356 | 356 | 5846 |
| siRNA 357 | 357 | 5847 |
| siRNA 364 | 364 | 5854 |
| siRNA 368 | 368 | 5858 |
| siRNA 370 | 370 | 5860 |
| siRNA 375 | 375 | 5865 |
| siRNA 376 | 376 | 5866 |
| siRNA 381 | 381 | 5871 |
| siRNA 394 | 394 | 5884 |
| siRNA 400 | 400 | 5890 |
| siRNA 403 | 403 | 5893 |
| siRNA 404 | 404 | 5894 |
| siRNA 405 | 405 | 5895 |
| siRNA 407 | 407 | 5897 |
| siRNA 415 | 415 | 5905 |
| siRNA 416 | 416 | 5906 |
| siRNA 422 | 422 | 5912 |
| siRNA 424 | 424 | 5914 |
| siRNA 428 | 428 | 5918 |
| siRNA 436 | 436 | 5926 |
| siRNA 457 | 457 | 5947 |
| siRNA 462 | 462 | 5952 |
| siRNA 463 | 463 | 5953 |
| siRNA 464 | 464 | 5954 |
| siRNA 465 | 465 | 5955 |
| siRNA 468 | 468 | 5958 |
| siRNA 482 | 482 | 5972 |
| siRNA 488 | 488 | 5978 |
| siRNA 493 | 493 | 5983 |
| siRNA 494 | 494 | 5984 |
| siRNA 509 | 509 | 5999 |
| siRNA 510 | 510 | 6000 |
| siRNA 514 | 514 | 6004 |
| siRNA 523 | 523 | 6013 |
| siRNA 526 | 526 | 6016 |
| siRNA 528 | 528 | 6018 |
| siRNA 536 | 536 | 6026 |
| siRNA 538 | 538 | 6028 |
| siRNA 539 | 539 | 6029 |
| siRNA 541 | 541 | 6031 |
| siRNA 542 | 542 | 6032 |
| siRNA 546 | 546 | 6036 |
| siRNA 547 | 547 | 6037 |
| siRNA 552 | 552 | 6042 |
| siRNA 556 | 556 | 6046 |
| siRNA 558 | 558 | 6048 |
| siRNA 562 | 562 | 6052 |
| siRNA 570 | 570 | 6060 |
| siRNA 573 | 573 | 6063 |
| siRNA 574 | 574 | 6064 |
| siRNA 575 | 575 | 6065 |
| siRNA 585 | 585 | 6075 |
| siRNA 587 | 587 | 6077 |
| siRNA 589 | 589 | 6079 |
| siRNA 596 | 596 | 6086 |
| siRNA 602 | 602 | 6092 |
| siRNA 614 | 614 | 6104 |
| siRNA 617 | 617 | 6107 |
| siRNA 619 | 619 | 6109 |
| siRNA 620 | 620 | 6110 |
| siRNA 621 | 621 | 6111 |
| siRNA 622 | 622 | 6112 |
| siRNA 623 | 623 | 6113 |
| siRNA 625 | 625 | 6115 |
| siRNA 627 | 627 | 6117 |
| siRNA 632 | 632 | 6122 |
| siRNA 633 | 633 | 6123 |
| siRNA 637 | 637 | 6127 |
| siRNA 638 | 638 | 6128 |
| siRNA 639 | 639 | 6129 |
| siRNA 640 | 640 | 6130 |
| siRNA 643 | 643 | 6133 |
| siRNA 644 | 644 | 6134 |
| siRNA 647 | 647 | 6137 |
| siRNA 650 | 650 | 6140 |
| siRNA 651 | 651 | 6141 |
| siRNA 652 | 652 | 6142 |
| siRNA 653 | 653 | 6143 |
| siRNA 656 | 656 | 6146 |
| siRNA 657 | 657 | 6147 |
| siRNA 660 | 660 | 6150 |
| siRNA 662 | 662 | 6152 |
| siRNA 663 | 663 | 6153 |
| siRNA 664 | 664 | 6154 |
| siRNA 665 | 665 | 6155 |
| siRNA 666 | 666 | 6156 |
| siRNA 671 | 671 | 6161 |
| siRNA 674 | 674 | 6164 |
| siRNA 678 | 678 | 6168 |
| siRNA 679 | 679 | 6169 |
| siRNA 680 | 680 | 6170 |
| siRNA 682 | 682 | 6172 |
| siRNA 683 | 683 | 6173 |
| siRNA 685 | 685 | 6175 |
| siRNA 689 | 689 | 6179 |
| siRNA 690 | 690 | 6180 |
| siRNA 691 | 691 | 6181 |
| siRNA 692 | 692 | 6182 |
| siRNA 719 | 719 | 6209 |
| siRNA 736 | 736 | 6226 |
| siRNA 737 | 737 | 6227 |
| siRNA 738 | 738 | 6228 |
| siRNA 739 | 739 | 6229 |
| siRNA 740 | 740 | 6230 |
| siRNA 743 | 743 | 6233 |
| siRNA 744 | 744 | 6234 |
| siRNA 749 | 749 | 6239 |
| siRNA 750 | 750 | 6240 |
| siRNA 763 | 763 | 6253 |
| siRNA 764 | 764 | 6254 |
| siRNA 766 | 766 | 6256 |
| siRNA 771 | 771 | 6261 |
| siRNA 772 | 772 | 6262 |
| siRNA 782 | 782 | 6272 |
| siRNA 783 | 783 | 6273 |
| siRNA 784 | 784 | 6274 |
| siRNA 785 | 785 | 6275 |
| siRNA 786 | 786 | 6276 |
| siRNA 787 | 787 | 6277 |
| siRNA 788 | 788 | 6278 |
| siRNA 789 | 789 | 6279 |
| siRNA 792 | 792 | 6282 |
| siRNA 795 | 795 | 6285 |
| siRNA 796 | 796 | 6286 |
| siRNA 797 | 797 | 6287 |
| siRNA 810 | 810 | 6300 |
| siRNA 813 | 813 | 6303 |
| siRNA 818 | 818 | 6308 |
| siRNA 819 | 819 | 6309 |
| siRNA 831 | 831 | 6321 |
| siRNA 853 | 853 | 6343 |
| siRNA 854 | 854 | 6344 |
| siRNA 856 | 856 | 6346 |
| siRNA 857 | 857 | 6347 |
| siRNA 858 | 858 | 6348 |
| siRNA 859 | 859 | 6349 |
| siRNA 872 | 872 | 6362 |
| siRNA 876 | 876 | 6366 |
| siRNA 877 | 877 | 6367 |
| siRNA 884 | 884 | 6374 |
| siRNA 887 | 887 | 6377 |
| siRNA 888 | 888 | 6378 |
| siRNA 889 | 889 | 6379 |
| siRNA 890 | 890 | 6380 |
| siRNA 894 | 894 | 6384 |
| siRNA 922 | 922 | 6412 |
| siRNA 924 | 924 | 6414 |
| siRNA 925 | 925 | 6415 |
| siRNA 926 | 926 | 6416 |
| siRNA 928 | 928 | 6418 |
| siRNA 930 | 930 | 6420 |
| siRNA 933 | 933 | 6423 |
| siRNA 934 | 934 | 6424 |
| siRNA 935 | 935 | 6425 |
| siRNA 936 | 936 | 6426 |
| siRNA 938 | 938 | 6428 |
| siRNA 941 | 941 | 6431 |
| siRNA 942 | 942 | 6432 |
| siRNA 953 | 953 | 6443 |
| siRNA 954 | 954 | 6444 |
| siRNA 959 | 959 | 6449 |
| siRNA 961 | 961 | 6451 |
| siRNA 967 | 967 | 6457 |
| siRNA 973 | 973 | 6463 |
| siRNA 981 | 981 | 6471 |
| siRNA 999 | 999 | 6489 |
| siRNA 1000 | 1000 | 6490 |
| siRNA 1001 | 1001 | 6491 |
| siRNA 1004 | 1004 | 6494 |
| siRNA 1006 | 1006 | 6496 |
| siRNA 1007 | 1007 | 6497 |
| siRNA 1036 | 1036 | 6526 |
| siRNA 1040 | 1040 | 6530 |
| siRNA 1050 | 1050 | 6540 |
| siRNA 1052 | 1052 | 6542 |
| siRNA 1053 | 1053 | 6543 |
| siRNA 1054 | 1054 | 6544 |
| siRNA 1056 | 1056 | 6546 |
| siRNA 1062 | 1062 | 6552 |
| siRNA 1067 | 1067 | 6557 |
| siRNA 1072 | 1072 | 6562 |
| siRNA 1075 | 1075 | 6565 |
| siRNA 1082 | 1082 | 6572 |
| siRNA 1084 | 1084 | 6574 |
| siRNA 1085 | 1085 | 6575 |
| siRNA 1086 | 1086 | 6576 |
| siRNA 1087 | 1087 | 6577 |
| siRNA 1134 | 1134 | 6624 |
| siRNA 1153 | 1153 | 6643 |
| siRNA 1154 | 1154 | 6644 |
| siRNA 1155 | 1155 | 6645 |
| siRNA 1156 | 1156 | 6646 |
| siRNA 1157 | 1157 | 6647 |
| siRNA 1158 | 1158 | 6648 |
| siRNA 1159 | 1159 | 6649 |
| siRNA 1175 | 1175 | 6665 |
| siRNA 1178 | 1178 | 6668 |
| siRNA 1185 | 1185 | 6675 |
| siRNA 1187 | 1187 | 6677 |
| siRNA 1191 | 1191 | 6681 |
| siRNA 1192 | 1192 | 6682 |
| siRNA 1196 | 1196 | 6686 |
| siRNA 1224 | 1224 | 6714 |
| siRNA 1242 | 1242 | 6732 |
| siRNA 1248 | 1248 | 6738 |
| siRNA 1249 | 1249 | 6739 |
| siRNA 1250 | 1250 | 6740 |
| siRNA 1251 | 1251 | 6741 |
| siRNA 1255 | 1255 | 6745 |
| siRNA 1279 | 1279 | 6769 |
| siRNA 1280 | 1280 | 5770 |
| siRNA 1282 | 1282 | 6772 |
| siRNA 1286 | 1286 | 6776 |
| siRNA 1288 | 1288 | 6778 |
| siRNA 1294 | 1294 | 6784 |
| siRNA 1296 | 1296 | 6786 |
| siRNA 1301 | 1301 | 6791 |
| siRNA 1302 | 1302 | 6792 |
| siRNA 1303 | 1303 | 6793 |
| siRNA 1304 | 1304 | 6794 |
| siRNA 1307 | 1307 | 6797 |
| siRNA 1309 | 1309 | 6799 |
| siRNA 1318 | 1318 | 6808 |
| siRNA 1319 | 1319 | 6809 |
| siRNA 1320 | 1320 | 6810 |
| siRNA 1321 | 1321 | 6811 |
| siRNA 1322 | 1322 | 6812 |
| siRNA 1326 | 1326 | 6816 |
| siRNA 1333 | 1333 | 6823 |
| siRNA 1339 | 1339 | 6829 |
| siRNA 1340 | 1340 | 6830 |
| siRNA 1342 | 1342 | 6832 |
| siRNA 1343 | 1343 | 6833 |
| siRNA 1344 | 1344 | 6834 |
| siRNA 1346 | 1346 | 6836 |
| siRNA 1347 | 1347 | 6837 |
| siRNA 1351 | 1351 | 6841 |
| siRNA 1357 | 1357 | 6847 |
| siRNA 1363 | 1363 | 6853 |
| siRNA 1365 | 1365 | 6855 |
| siRNA 1372 | 1372 | 6862 |
| siRNA 1395 | 1395 | 6885 |
| siRNA 1398 | 1398 | 6888 |
| siRNA 1405 | 1405 | 6895 |
| siRNA 1406 | 1406 | 6896 |
| siRNA 1408 | 1408 | 6898 |
| siRNA 1410 | 1410 | 6900 |
| siRNA 1411 | 1411 | 6901 |
| siRNA 1422 | 1422 | 6912 |
| siRNA 1430 | 1430 | 6920 |
| siRNA 1431 | 1431 | 6921 |
| siRNA 1434 | 1434 | 6924 |
| siRNA 1435 | 1435 | 6925 |
| siRNA 1441 | 1441 | 6931 |
| siRNA 1442 | 1442 | 6932 |
| siRNA 1445 | 1445 | 6935 |
| siRNA 1448 | 1448 | 6938 |
| siRNA 1449 | 1449 | 6939 |
| siRNA 1452 | 1452 | 6942 |
| siRNA 1453 | 1453 | 6943 |
| siRNA 1459 | 1459 | 6949 |
| siRNA 1460 | 1460 | 6950 |
| siRNA 1461 | 1461 | 6951 |
| siRNA 1462 | 1462 | 6952 |
| siRNA 1463 | 1463 | 6953 |
| siRNA 1465 | 1465 | 6955 |
| siRNA 1466 | 1466 | 6956 |
| siRNA 1467 | 1467 | 6957 |
| siRNA 1468 | 1468 | 6958 |
| siRNA 1469 | 1469 | 6959 |
| siRNA 1470 | 1470 | 6960 |
| siRNA 1472 | 1472 | 6962 |
| siRNA 1473 | 1473 | 6963 |
| siRNA 1474 | 1474 | 6964 |
| siRNA 1475 | 1475 | 6965 |
| siRNA 1476 | 1476 | 6966 |
| siRNA 1477 | 1477 | 6967 |
| siRNA 1481 | 1481 | 6971 |
| siRNA 1494 | 1494 | 6984 |
| siRNA 1495 | 1495 | 6985 |
| siRNA 1498 | 1498 | 6988 |
| siRNA 1503 | 1503 | 6993 |
| siRNA 1504 | 1504 | 6994 |
| siRNA 1505 | 1505 | 6995 |
| siRNA 1510 | 1510 | 7000 |
| siRNA 1521 | 1521 | 7011 |
| siRNA 1529 | 1529 | 7019 |
| siRNA 1533 | 1533 | 7023 |
| siRNA 1535 | 1535 | 7025 |
| siRNA 1560 | 1560 | 7050 |
| siRNA 1568 | 1568 | 7058 |
| siRNA 1569 | 1569 | 7059 |
| siRNA 1570 | 1570 | 7060 |
| siRNA 1572 | 1572 | 7062 |
| siRNA 1575 | 1575 | 7065 |
| siRNA 1587 | 1587 | 7077 |
| siRNA 1588 | 1588 | 7078 |
| siRNA 1589 | 1589 | 7079 |
| siRNA 1596 | 1596 | 7086 |
| siRNA 1614 | 1614 | 7104 |
| siRNA 1616 | 1616 | 7106 |
| siRNA 1621 | 1621 | 7111 |
| siRNA 1622 | 1622 | 7112 |
| siRNA 1623 | 1623 | 7113 |
| siRNA 1627 | 1627 | 7117 |
| siRNA 1628 | 1628 | 7118 |
| siRNA 1631 | 1631 | 7121 |
| siRNA 1632 | 1632 | 7122 |
| siRNA 1633 | 1633 | 7123 |
| siRNA 1634 | 1634 | 7124 |
| siRNA 1635 | 1635 | 7125 |
| siRNA 1636 | 1636 | 7126 |
| siRNA 1637 | 1637 | 7127 |
| siRNA 1638 | 1638 | 7128 |
| siRNA 1641 | 1641 | 7131 |
| siRNA 1643 | 1643 | 7133 |
| siRNA 1644 | 1644 | 7134 |
| siRNA 1645 | 1645 | 7135 |
| siRNA 1648 | 1648 | 7138 |
| siRNA 1649 | 1649 | 7139 |
| siRNA 1650 | 1650 | 7140 |
| siRNA 1651 | 1651 | 7141 |
| siRNA 1652 | 1652 | 7142 |
| siRNA 1654 | 1654 | 7144 |
| siRNA 1655 | 1655 | 7145 |
| siRNA 1656 | 1656 | 7146 |
| siRNA 1658 | 1658 | 7148 |
| siRNA 1659 | 1659 | 7149 |
| siRNA 1683 | 1683 | 7173 |
| siRNA 1684 | 1684 | 7174 |
| siRNA 1689 | 1689 | 7179 |
| siRNA 1691 | 1691 | 7181 |
| siRNA 1693 | 1693 | 7183 |
| siRNA 1697 | 1697 | 7187 |
| siRNA 1699 | 1699 | 7189 |
| siRNA 1703 | 1703 | 7193 |
| siRNA 1706 | 1706 | 7196 |
| siRNA 1707 | 1707 | 7197 |
| siRNA 1708 | 1708 | 7198 |
| siRNA 1712 | 1712 | 7202 |
| siRNA 1720 | 1720 | 7210 |
| siRNA 1763 | 1763 | 7253 |
| siRNA 1802 | 1802 | 7292 |
| siRNA 1805 | 1805 | 7295 |
| siRNA 1806 | 1806 | 7296 |
| siRNA 1811 | 1811 | 7301 |
| siRNA 1812 | 1812 | 7302 |
| siRNA 1814 | 1814 | 7304 |
| siRNA 1816 | 1816 | 7306 |
| siRNA 1821 | 1821 | 7311 |
| siRNA 1825 | 1825 | 7315 |
| siRNA 1826 | 1826 | 7316 |
| siRNA 1832 | 1832 | 7322 |
| siRNA 1834 | 1834 | 7324 |
| siRNA 1844 | 1844 | 7334 |
| siRNA 1846 | 1846 | 7336 |
| siRNA 1847 | 1847 | 7337 |
| siRNA 1851 | 1851 | 7341 |
| siRNA 1857 | 1857 | 7347 |
| siRNA 1861 | 1861 | 7351 |
| siRNA 1865 | 1865 | 7355 |
| siRNA 1897 | 1897 | 7387 |
| siRNA 1909 | 1909 | 7399 |
| siRNA 1912 | 1912 | 7402 |
| siRNA 1914 | 1914 | 7404 |
| siRNA 1915 | 1915 | 7405 |
| siRNA 1916 | 1916 | 7406 |
| siRNA 1917 | 1917 | 7407 |
| siRNA 1918 | 1918 | 7408 |
| siRNA 1919 | 1919 | 7409 |
| siRNA 1920 | 1920 | 7410 |
| siRNA 1921 | 1921 | 7411 |
| siRNA 1922 | 1922 | 7412 |
| siRNA 1923 | 1923 | 7413 |
| siRNA 1924 | 1924 | 7414 |
| siRNA 1926 | 1926 | 7416 |
| siRNA 1927 | 1927 | 7417 |
| siRNA 1928 | 1928 | 7418 |
| siRNA 1929 | 1929 | 7419 |
| siRNA 1934 | 1934 | 7424 |
| siRNA 1935 | 1935 | 7425 |
| siRNA 1936 | 1936 | 7426 |
| siRNA 1937 | 1937 | 7427 |
| siRNA 1938 | 1938 | 7428 |
| siRNA 1940 | 1940 | 7430 |
| siRNA 1941 | 1941 | 7431 |
| siRNA 1948 | 1948 | 7438 |
| siRNA 1951 | 1951 | 7441 |
| siRNA 1977 | 1977 | 7467 |
| siRNA 1980 | 1980 | 7470 |
| siRNA 1981 | 1981 | 7471 |
| siRNA 1984 | 1984 | 7474 |
| siRNA 1985 | 1985 | 7475 |
| siRNA 1986 | 1986 | 7476 |
| siRNA 1987 | 1987 | 7477 |
| siRNA 1988 | 1988 | 7478 |
| siRNA 1989 | 1989 | 7479 |
| siRNA 1991 | 1991 | 7481 |
| siRNA 1992 | 1992 | 7482 |
| siRNA 1994 | 1994 | 7484 |
| siRNA 1995 | 1995 | 7485 |
| siRNA 1996 | 1996 | 7486 |
| siRNA 1999 | 1999 | 7489 |
| siRNA 2000 | 2000 | 7490 |
| siRNA 2002 | 2002 | 7492 |
| siRNA 2005 | 2005 | 7495 |
| siRNA 2006 | 2006 | 7496 |
| siRNA 2007 | 2007 | 7497 |
| siRNA 2014 | 2014 | 7504 |
| siRNA 2020 | 2020 | 7510 |
| siRNA 2049 | 2049 | 7539 |
| siRNA 2051 | 2051 | 7541 |
| siRNA 2056 | 2056 | 7546 |
| siRNA 2057 | 2057 | 7547 |
| siRNA 2062 | 2062 | 7552 |
| siRNA 2065 | 2065 | 7555 |
| siRNA 2071 | 2071 | 7561 |
| siRNA 2073 | 2073 | 7563 |
| siRNA 2074 | 2074 | 7564 |
| siRNA 2077 | 2077 | 7567 |
| siRNA 2088 | 2088 | 7578 |
| siRNA 2096 | 2096 | 7586 |
| siRNA 2109 | 2109 | 7599 |
| siRNA 2112 | 2112 | 7602 |
| siRNA 2116 | 2116 | 7606 |
| siRNA 2120 | 2120 | 7610 |
| siRNA 2142 | 2142 | 7632 |
| siRNA 2147 | 2147 | 7637 |
| siRNA 2148 | 2148 | 7638 |
| siRNA 2149 | 2149 | 7639 |
| siRNA 2150 | 2150 | 7640 |
| siRNA 2151 | 2151 | 7641 |
| siRNA 2152 | 2152 | 7642 |
| siRNA 2154 | 2154 | 7644 |
| siRNA 2157 | 2157 | 7647 |
| siRNA 2158 | 2158 | 7648 |
| siRNA 2161 | 2161 | 7651 |
| siRNA 2164 | 2164 | 7654 |
| siRNA 2167 | 2167 | 7657 |
| siRNA 2168 | 2168 | 7658 |
| siRNA 2170 | 2170 | 7660 |
| siRNA 2171 | 2171 | 7661 |
| siRNA 2172 | 2172 | 7662 |
| siRNA 2175 | 2175 | 7665 |
| siRNA 2193 | 2193 | 7683 |
| siRNA 2202 | 2202 | 7692 |
| siRNA 2209 | 2209 | 7699 |
| siRNA 2213 | 2213 | 7703 |
| siRNA 2214 | 2214 | 7704 |
| siRNA 2216 | 2216 | 7706 |
| siRNA 2219 | 2219 | 7709 |
| siRNA 2220 | 2220 | 7710 |
| siRNA 2244 | 2244 | 7734 |
| siRNA 2245 | 2245 | 7735 |
| siRNA 2246 | 2246 | 7736 |
| siRNA 2250 | 2250 | 7740 |
| siRNA 2251 | 2251 | 7741 |
| siRNA 2252 | 2252 | 7742 |
| siRNA 2254 | 2254 | 7744 |
| siRNA 2257 | 2257 | 7747 |
| siRNA 2258 | 2258 | 7748 |
| siRNA 2259 | 2259 | 7749 |
| siRNA 2286 | 2286 | 7776 |
| siRNA 2287 | 2287 | 7777 |
| siRNA 2292 | 2292 | 7782 |
| siRNA 2294 | 2294 | 7784 |
| siRNA 2306 | 2306 | 7796 |
| siRNA 2307 | 2307 | 7797 |
| siRNA 2310 | 2310 | 7800 |
| siRNA 2311 | 2311 | 7801 |
| siRNA 2312 | 2312 | 7802 |
| siRNA 2313 | 2313 | 7803 |
| siRNA 2314 | 2314 | 7804 |
| siRNA 2318 | 2318 | 7808 |
| siRNA 2322 | 2322 | 7812 |
| siRNA 2324 | 2324 | 7814 |
| siRNA 2328 | 2328 | 7818 |
| siRNA 2329 | 2329 | 7819 |
| siRNA 2332 | 2332 | 7822 |
| siRNA 2336 | 2336 | 7826 |
| siRNA 2338 | 2338 | 7828 |
| siRNA 2339 | 2339 | 7829 |
| siRNA 2340 | 2340 | 7830 |
| siRNA 2355 | 2355 | 7845 |
| siRNA 2356 | 2356 | 7846 |
| siRNA 2357 | 2357 | 7847 |
| siRNA 2360 | 2360 | 7850 |
| siRNA 2362 | 2362 | 7852 |
| siRNA 2364 | 2364 | 7854 |
| siRNA 2365 | 2365 | 7855 |
| siRNA 2366 | 2366 | 7856 |
| siRNA 2368 | 2368 | 7858 |
| siRNA 2369 | 2369 | 7859 |
| siRNA 2370 | 2370 | 7860 |
| siRNA 2393 | 2393 | 7883 |
| siRNA 2403 | 2403 | 7893 |
| siRNA 2417 | 2417 | 7907 |
| siRNA 2418 | 2418 | 7908 |
| siRNA 2436 | 2436 | 7926 |
| siRNA 2437 | 2437 | 7927 |
| siRNA 2467 | 2467 | 7957 |
| siRNA 2470 | 2470 | 7960 |
| siRNA 2471 | 2471 | 7961 |
| siRNA 2472 | 2472 | 7962 |
| siRNA 2477 | 2477 | 7967 |
| siRNA 2480 | 2480 | 7970 |
| siRNA 2483 | 2483 | 7973 |
| siRNA 2486 | 2486 | 7976 |
| siRNA 2488 | 2488 | 7978 |
| siRNA 2489 | 2489 | 7979 |
| siRNA 2490 | 2490 | 7980 |
| siRNA 2528 | 2528 | 8018 |
| siRNA 2530 | 2530 | 8020 |
| siRNA 2531 | 2531 | 8021 |
| siRNA 2532 | 2532 | 8022 |
| siRNA 2548 | 2548 | 8038 |
| siRNA 2559 | 2559 | 8049 |
| siRNA 2565 | 2565 | 8055 |
| siRNA 2568 | 2568 | 8058 |
| siRNA 2575 | 2575 | 8065 |
| siRNA 2577 | 2577 | 8067 |
| siRNA 2598 | 2598 | 8088 |
| siRNA 2610 | 2610 | 8100 |
| siRNA 2611 | 2611 | 8101 |
| siRNA 2616 | 2616 | 8106 |
| siRNA 2619 | 2619 | 8109 |
| siRNA 2620 | 2620 | 8110 |
| siRNA 2623 | 2623 | 8113 |
| siRNA 2630 | 2630 | 8120 |
| siRNA 2631 | 2631 | 8121 |
| siRNA 2632 | 2632 | 8122 |
| siRNA 2633 | 2633 | 8123 |
| siRNA 2637 | 2637 | 8127 |
| siRNA 2640 | 2640 | 8130 |
| siRNA 2642 | 2642 | 8132 |
| siRNA 2644 | 2644 | 8134 |
| siRNA 2645 | 2645 | 8135 |
| siRNA 2646 | 2646 | 8136 |
| siRNA 2673 | 2673 | 8163 |
| siRNA 2675 | 2675 | 8165 |
| siRNA 2679 | 2679 | 8169 |
| siRNA 2680 | 2680 | 8170 |
| siRNA 2682 | 2682 | 8172 |
| siRNA 2683 | 2683 | 8173 |
| siRNA 2688 | 2688 | 8178 |
| siRNA 2690 | 2690 | 8180 |
| siRNA 2691 | 2691 | 8181 |
| siRNA 2699 | 2699 | 8189 |
| siRNA 2708 | 2708 | 8198 |
| siRNA 2713 | 2713 | 8203 |
| siRNA 2755 | 2755 | 8245 |
| siRNA 2756 | 2756 | 8246 |
| siRNA 2757 | 2757 | 8247 |
| siRNA 2759 | 2759 | 8249 |
| siRNA 2760 | 2760 | 8250 |
| siRNA 2761 | 2761 | 8251 |
| siRNA 2762 | 2762 | 8252 |
| siRNA 2763 | 2763 | 8253 |
| siRNA 2764 | 2764 | 8254 |
| siRNA 2767 | 2767 | 8257 |
| siRNA 2815 | 2815 | 8305 |
| siRNA 2816 | 2816 | 8306 |
| siRNA 2817 | 2817 | 8307 |
| siRNA 2821 | 2821 | 8311 |
| siRNA 2822 | 2822 | 8312 |
| siRNA 2825 | 2825 | 8315 |
| siRNA 2828 | 2828 | 8318 |
| siRNA 2829 | 2829 | 8319 |
| siRNA 2830 | 2830 | 8320 |
| siRNA 2833 | 2833 | 8323 |
| siRNA 2834 | 2834 | 8324 |
| siRNA 2836 | 2836 | 8326 |
| siRNA 2841 | 2841 | 8331 |
| siRNA 2842 | 2842 | 8332 |
| siRNA 2847 | 2847 | 8337 |
| siRNA 2866 | 2866 | 8356 |
| siRNA 2882 | 2882 | 8372 |
| siRNA 2883 | 2883 | 8373 |
| siRNA 2884 | 2884 | 8374 |
| siRNA 2886 | 2886 | 8376 |
| siRNA 2887 | 2887 | 8377 |
| siRNA 2888 | 2888 | 8378 |
| siRNA 2890 | 2890 | 8380 |
| siRNA 2897 | 2897 | 8387 |
| siRNA 2898 | 2898 | 8388 |
| siRNA 2902 | 2902 | 8392 |
| siRNA 2903 | 2903 | 8393 |
| siRNA 2908 | 2908 | 8398 |
| siRNA 2917 | 2917 | 8407 |
| siRNA 2926 | 2926 | 8416 |
| siRNA 2928 | 2928 | 8418 |
| siRNA 2929 | 2929 | 8419 |
| siRNA 2930 | 2930 | 8420 |
| siRNA 2932 | 2932 | 8422 |
| siRNA 2933 | 2933 | 8423 |
| siRNA 2937 | 2937 | 8427 |
| siRNA 2938 | 2938 | 8428 |
| siRNA 2939 | 2939 | 8429 |
| siRNA 2940 | 2940 | 8430 |
| siRNA 2942 | 2942 | 8432 |
| siRNA 2944 | 2944 | 8434 |
| siRNA 2946 | 2946 | 8436 |
| siRNA 2947 | 2947 | 8437 |
| siRNA 2949 | 2949 | 8439 |
| siRNA 2952 | 2952 | 8442 |
| siRNA 2981 | 2981 | 8471 |
| siRNA 2982 | 2982 | 8472 |
| siRNA 2983 | 2983 | 8473 |
| siRNA 2984 | 2984 | 8474 |
| siRNA 2985 | 2985 | 8475 |
| siRNA 2986 | 2986 | 8476 |
| siRNA 2987 | 2987 | 8477 |
| siRNA 2998 | 2998 | 8488 |
| siRNA 3006 | 3006 | 8496 |
| siRNA 3024 | 3024 | 8514 |
| siRNA 3025 | 3025 | 8515 |
| siRNA 3035 | 3035 | 8525 |
| siRNA 3038 | 3038 | 8528 |
| siRNA 3042 | 3042 | 8532 |
| siRNA 3046 | 3046 | 8536 |
| siRNA 3053 | 3053 | 8543 |
| siRNA 3091 | 3091 | 8581 |
| siRNA 3093 | 3093 | 8583 |
| siRNA 3094 | 3094 | 8584 |
| siRNA 3098 | 3098 | 8588 |
| siRNA 3099 | 3099 | 8589 |
| siRNA 3108 | 3108 | 8598 |
| siRNA 3140 | 3140 | 8630 |
| siRNA 3146 | 3146 | 8636 |
| siRNA 3156 | 3156 | 8646 |
| siRNA 3164 | 3164 | 8654 |
| siRNA 3200 | 3200 | 8690 |
| siRNA 3209 | 3209 | 8699 |
| siRNA 3218 | 3218 | 8708 |
| siRNA 3221 | 3221 | 8711 |
| siRNA 3222 | 3222 | 8712 |
| siRNA 3223 | 3223 | 8713 |
| siRNA 3224 | 3224 | 8714 |
| siRNA 3230 | 3230 | 8720 |
| siRNA 3231 | 3231 | 8721 |
| siRNA 3232 | 3232 | 8722 |
| siRNA 3238 | 3238 | 8728 |
| siRNA 3247 | 3247 | 8737 |
| siRNA 3259 | 3259 | 8749 |
| siRNA 3261 | 3261 | 8751 |
| siRNA 3262 | 3262 | 8752 |
| siRNA 3263 | 3263 | 8753 |
| siRNA 3276 | 3276 | 8766 |
| siRNA 3277 | 3277 | 8767 |
| siRNA 3305 | 3305 | 8795 |
| siRNA 3310 | 3310 | 8800 |
| siRNA 3315 | 3315 | 8805 |
| siRNA 3330 | 3330 | 8820 |
| siRNA 3333 | 3333 | 8823 |
| siRNA 3334 | 3334 | 8824 |
| siRNA 3335 | 3335 | 8825 |
| siRNA 3339 | 3339 | 8829 |
| siRNA 3342 | 3342 | 8832 |
| siRNA 3349 | 3349 | 8839 |
| siRNA 3350 | 3350 | 8840 |
| siRNA 3351 | 3351 | 8841 |
| siRNA 3352 | 3352 | 8842 |
| siRNA 3353 | 3353 | 8843 |
| siRNA 3354 | 3354 | 8844 |
| siRNA 3355 | 3355 | 8845 |
| siRNA 3357 | 3357 | 8847 |
| siRNA 3358 | 3358 | 8848 |
| siRNA 3359 | 3359 | 8849 |
| siRNA 3360 | 3360 | 8850 |
| siRNA 3364 | 3364 | 8854 |
| siRNA 3396 | 3396 | 8886 |
| siRNA 3397 | 3397 | 8887 |
| siRNA 3403 | 3403 | 8893 |
| siRNA 3405 | 3405 | 8895 |
| siRNA 3406 | 3406 | 8896 |
| siRNA 3407 | 3407 | 8897 |
| siRNA 3410 | 3410 | 8900 |
| siRNA 3411 | 3411 | 8901 |
| siRNA 3412 | 3412 | 8902 |
| siRNA 3413 | 3413 | 8903 |
| siRNA 3415 | 3415 | 8905 |
| siRNA 3416 | 3416 | 8906 |
| siRNA 3417 | 3417 | 8907 |
| siRNA 3418 | 3418 | 8908 |
| siRNA 3425 | 3425 | 8915 |
| siRNA 3431 | 3431 | 8921 |
| siRNA 3432 | 3432 | 8922 |
| siRNA 3435 | 3435 | 8925 |
| siRNA 3436 | 3436 | 8926 |
| siRNA 3437 | 3437 | 8927 |
| siRNA 3438 | 3438 | 8928 |
| siRNA 3439 | 3439 | 8929 |
| siRNA 3443 | 3443 | 8933 |
| siRNA 3444 | 3444 | 8934 |
| siRNA 3449 | 3449 | 8939 |
| siRNA 3450 | 3450 | 8940 |
| siRNA 3451 | 3451 | 8941 |
| siRNA 3456 | 3456 | 8946 |
| siRNA 3457 | 3457 | 8947 |
| siRNA 3458 | 3458 | 8948 |
| siRNA 3460 | 3460 | 8950 |
| siRNA 3461 | 3461 | 8951 |
| siRNA 3470 | 3470 | 8960 |
| siRNA 3471 | 3471 | 8961 |
| siRNA 3472 | 3472 | 8962 |
| siRNA 3484 | 3484 | 8974 |
| siRNA 3487 | 3487 | 8977 |
| siRNA 3488 | 3488 | 8978 |
| siRNA 3489 | 3489 | 8979 |
| siRNA 3490 | 3490 | 8980 |
| siRNA 3491 | 3491 | 8981 |
| siRNA 3492 | 3492 | 8982 |
| siRNA 3494 | 3494 | 8984 |
| siRNA 3495 | 3495 | 8985 |
| siRNA 3500 | 3500 | 8990 |
| siRNA 3502 | 3502 | 8992 |
| siRNA 3504 | 3504 | 8994 |
| siRNA 3511 | 3511 | 9001 |
| siRNA 3512 | 3512 | 9002 |
| siRNA 3516 | 3516 | 9006 |
| siRNA 3517 | 3517 | 9007 |
| siRNA 3540 | 3540 | 9030 |
| siRNA 3546 | 3546 | 9036 |
| siRNA 3549 | 3549 | 9039 |
| siRNA 3558 | 3558 | 9048 |
| siRNA 3560 | 3560 | 9050 |
| siRNA 3561 | 3561 | 9051 |
| siRNA 3564 | 3564 | 9054 |
| siRNA 3566 | 3566 | 9056 |
| siRNA 3576 | 3576 | 9066 |
| siRNA 3582 | 3582 | 9072 |
| siRNA 3583 | 3583 | 9073 |
| siRNA 3585 | 3585 | 9075 |
| siRNA 3586 | 3586 | 9076 |
| siRNA 3587 | 3587 | 9077 |
| siRNA 3602 | 3602 | 9092 |
| siRNA 3604 | 3604 | 9094 |
| siRNA 3605 | 3605 | 9095 |
| siRNA 3610 | 3610 | 9100 |
| siRNA 3619 | 3619 | 9109 |
| siRNA 3629 | 3629 | 9119 |
| siRNA 3634 | 3634 | 9124 |
| siRNA 3635 | 3635 | 9125 |
| siRNA 3637 | 3637 | 9127 |
| siRNA 3638 | 3638 | 9128 |
| siRNA 3639 | 3639 | 9129 |
| siRNA 3640 | 3640 | 9130 |
| siRNA 3641 | 3641 | 9131 |
| siRNA 3663 | 3663 | 9153 |
| siRNA 3677 | 3677 | 9167 |
| siRNA 3685 | 3685 | 9175 |
| siRNA 3686 | 3686 | 9176 |
| siRNA 3688 | 3688 | 9178 |
| siRNA 3691 | 3691 | 9181 |
| siRNA 3692 | 3692 | 9182 |
| siRNA 3695 | 3695 | 9185 |
| siRNA 3703 | 3703 | 9193 |
| siRNA 3709 | 3709 | 9199 |
| siRNA 3712 | 3712 | 9202 |
| siRNA 3746 | 3746 | 9236 |
| siRNA 3747 | 3747 | 9237 |
| siRNA 3748 | 3748 | 9238 |
| siRNA 3756 | 3756 | 9246 |
| siRNA 3792 | 3792 | 9282 |
| siRNA 3802 | 3802 | 9292 |
| siRNA 3803 | 3803 | 9293 |
| siRNA 3807 | 3807 | 9297 |
| siRNA 3810 | 3810 | 9300 |
| siRNA 3816 | 3816 | 9306 |
| siRNA 3838 | 3838 | 9328 |
| siRNA 3840 | 3840 | 9330 |
| siRNA 3841 | 3841 | 9331 |
| siRNA 3868 | 3868 | 9358 |
| siRNA 3869 | 3869 | 9359 |
| siRNA 3874 | 3874 | 9364 |
| siRNA 3875 | 3875 | 9365 |
| siRNA 3883 | 3883 | 9373 |
| siRNA 3897 | 3897 | 9387 |
| siRNA 3908 | 3908 | 9398 |
| siRNA 3909 | 3909 | 9399 |
| siRNA 3911 | 3911 | 9401 |
| siRNA 3912 | 3912 | 9402 |
| siRNA 3922 | 3922 | 9412 |
| siRNA 3926 | 3926 | 9416 |
| siRNA 3934 | 3934 | 9424 |
| siRNA 3936 | 3936 | 9426 |
| siRNA 3943 | 3943 | 9433 |
| siRNA 3944 | 3944 | 9434 |
| siRNA 3945 | 3945 | 9435 |
| siRNA 3960 | 3960 | 9450 |
| siRNA 3969 | 3969 | 9459 |
| siRNA 3970 | 3970 | 9460 |
| siRNA 3988 | 3988 | 9478 |
| siRNA 3991 | 3991 | 9481 |
| siRNA 4010 | 4010 | 9500 |
| siRNA 4025 | 4025 | 9515 |
| siRNA 4027 | 4027 | 9517 |
| siRNA 4036 | 4036 | 9526 |
| siRNA 4038 | 4038 | 9528 |
| siRNA 4040 | 4040 | 9530 |
| siRNA 4041 | 4041 | 9531 |
| siRNA 4043 | 4043 | 9533 |
| siRNA 4044 | 4044 | 9534 |
| siRNA 4047 | 4047 | 9537 |
| siRNA 4048 | 4048 | 9538 |
| siRNA 4049 | 4049 | 9539 |
| siRNA 4050 | 4050 | 9540 |
| siRNA 4051 | 4051 | 9541 |
| siRNA 4052 | 4052 | 9542 |
| siRNA 4053 | 4053 | 9543 |
| siRNA 4054 | 4054 | 9544 |
| siRNA 4056 | 4056 | 9546 |
| siRNA 4058 | 4058 | 9548 |
| siRNA 4064 | 4064 | 9554 |
| siRNA 4068 | 4068 | 9558 |
| siRNA 4070 | 4070 | 9560 |
| siRNA 4077 | 4077 | 9567 |
| siRNA 4083 | 4083 | 9573 |
| siRNA 4111 | 4111 | 9601 |
| siRNA 4112 | 4112 | 9602 |
| siRNA 4117 | 4117 | 9607 |
| siRNA 4122 | 4122 | 9612 |
| siRNA 4125 | 4125 | 9615 |
| siRNA 4132 | 4132 | 9622 |
| siRNA 4133 | 4133 | 9623 |
| siRNA 4138 | 4138 | 9628 |
| siRNA 4169 | 4169 | 9659 |
| siRNA 4179 | 4179 | 9669 |
| siRNA 4190 | 4190 | 9680 |
| siRNA 4192 | 4192 | 9682 |
| siRNA 4199 | 4199 | 9689 |
| siRNA 4207 | 4207 | 9697 |
| siRNA 4209 | 4209 | 9699 |
| siRNA 4210 | 4210 | 9700 |
| siRNA 4211 | 4211 | 9701 |
| siRNA 4212 | 4212 | 9702 |
| siRNA 4213 | 4213 | 9703 |
| siRNA 4214 | 4214 | 9704 |
| siRNA 4216 | 4216 | 9706 |
| siRNA 4218 | 4218 | 9708 |
| siRNA 4219 | 4219 | 9709 |
| siRNA 4222 | 4222 | 9712 |
| siRNA 4223 | 4223 | 9713 |
| siRNA 4224 | 4224 | 9714 |
| siRNA 4225 | 4225 | 9715 |
| siRNA 4226 | 4226 | 9716 |
| siRNA 4228 | 4228 | 9718 |
| siRNA 4233 | 4233 | 9723 |
| siRNA 4235 | 4235 | 9725 |
| siRNA 4237 | 4237 | 9727 |
| siRNA 4238 | 4238 | 9728 |
| siRNA 4242 | 4242 | 9732 |
| siRNA 4243 | 4243 | 9733 |
| siRNA 4245 | 4245 | 9735 |
| siRNA 4247 | 4247 | 9737 |
| siRNA 4252 | 4252 | 9742 |
| siRNA 4254 | 4254 | 9744 |
| siRNA 4275 | 4275 | 9765 |
| siRNA 4276 | 4276 | 9766 |
| siRNA 4277 | 4277 | 9767 |
| siRNA 4279 | 4279 | 9769 |
| siRNA 4282 | 4282 | 9772 |
| siRNA 4283 | 4283 | 9773 |
| siRNA 4284 | 4284 | 9774 |
| siRNA 4290 | 4290 | 9780 |
| siRNA 4292 | 4292 | 9782 |
| siRNA 4293 | 4293 | 9783 |
| siRNA 4294 | 4294 | 9784 |
| siRNA 4295 | 4295 | 9785 |
| siRNA 4296 | 4296 | 9786 |
| siRNA 4297 | 4297 | 9787 |
| siRNA 4305 | 4305 | 9795 |
| siRNA 4309 | 4309 | 9799 |
| siRNA 4313 | 4313 | 9803 |
| siRNA 4319 | 4319 | 9809 |
| siRNA 4322 | 4322 | 9812 |
| siRNA 4326 | 4326 | 9816 |
| siRNA 4351 | 4351 | 9841 |
| siRNA 4356 | 4356 | 9846 |
| siRNA 4368 | 4368 | 9858 |
| siRNA 4382 | 4382 | 9872 |
| siRNA 4388 | 4388 | 9878 |
| siRNA 4409 | 4409 | 9899 |
| siRNA 4414 | 4414 | 9904 |
| siRNA 4453 | 4453 | 9943 |
| siRNA 4455 | 4455 | 9945 |
| siRNA 4456 | 4456 | 9946 |
| siRNA 4459 | 4459 | 9949 |
| siRNA 4460 | 4460 | 9950 |
| siRNA 4467 | 4467 | 9957 |
| siRNA 4468 | 4468 | 9958 |
| siRNA 4474 | 4474 | 9964 |
| siRNA 4475 | 4475 | 9965 |
| siRNA 4481 | 4481 | 9971 |
| siRNA 4482 | 4482 | 9972 |
| siRNA 4487 | 4487 | 9977 |
| siRNA 4488 | 4488 | 9978 |
| siRNA 4489 | 4489 | 9979 |
| siRNA 4490 | 4490 | 9980 |
| siRNA 4491 | 4491 | 9981 |
| siRNA 4492 | 4492 | 9982 |
| siRNA 4496 | 4496 | 9986 |
| siRNA 4518 | 4518 | 10008 |
| siRNA 4519 | 4519 | 10009 |
| siRNA 4521 | 4521 | 10011 |
| siRNA 4527 | 4527 | 10017 |
| siRNA 4545 | 4545 | 10035 |
| siRNA 4547 | 4547 | 10037 |
| siRNA 4551 | 4551 | 10041 |
| siRNA 4552 | 4552 | 10042 |
| siRNA 4561 | 4561 | 10051 |
| siRNA 4565 | 4565 | 10055 |
| siRNA 4576 | 4576 | 10066 |
| siRNA 4584 | 4584 | 10074 |
| siRNA 4593 | 4593 | 10083 |
| siRNA 4594 | 4594 | 10084 |
| siRNA 4595 | 4595 | 10085 |
| siRNA 4596 | 4596 | 10086 |
| siRNA 4597 | 4597 | 10087 |
| siRNA 4598 | 4598 | 10088 |
| siRNA 4599 | 4599 | 10089 |
| siRNA 4600 | 4600 | 10090 |
| siRNA 4601 | 4601 | 10091 |
| siRNA 4628 | 4628 | 10118 |
| siRNA 4629 | 4629 | 10119 |
| siRNA 4644 | 4644 | 10134 |
| siRNA 4674 | 4674 | 10164 |
| siRNA 4675 | 4675 | 10165 |
| siRNA 4679 | 4679 | 10169 |
| siRNA 4680 | 4680 | 10170 |
| siRNA 4682 | 4682 | 10172 |
| siRNA 4705 | 4705 | 10195 |
| siRNA 4726 | 4726 | 10216 |
| siRNA 4739 | 4739 | 10229 |
| siRNA 4742 | 4742 | 10232 |
| siRNA 4749 | 4749 | 10239 |
| siRNA 4754 | 4754 | 10244 |
| siRNA 4756 | 4756 | 10246 |
| siRNA 4758 | 4758 | 10248 |
| siRNA 4763 | 4763 | 10253 |
| siRNA 4765 | 4765 | 10255 |
| siRNA 4767 | 4767 | 10257 |
| siRNA 4770 | 4770 | 10260 |
| siRNA 4788 | 4788 | 10278 |
| siRNA 4797 | 4797 | 10287 |
| siRNA 4799 | 4799 | 10289 |
| siRNA 4800 | 4800 | 10290 |
| siRNA 4802 | 4802 | 10292 |
| siRNA 4805 | 4805 | 10295 |
| siRNA 4807 | 4807 | 10297 |
| siRNA 4852 | 4852 | 10342 |
| siRNA 4862 | 4862 | 10352 |
| siRNA 4863 | 4863 | 10353 |
| siRNA 4902 | 4902 | 10392 |
| siRNA 4903 | 4903 | 10393 |
| siRNA 4905 | 4905 | 10395 |
| siRNA 4906 | 4906 | 10396 |
| siRNA 4910 | 4910 | 10400 |
| siRNA 4911 | 4911 | 10401 |
| siRNA 4915 | 4915 | 10405 |
| siRNA 4932 | 4932 | 10422 |
| siRNA 4934 | 4934 | 10424 |
| siRNA 4935 | 4935 | 10425 |
| siRNA 4936 | 4936 | 10426 |
| siRNA 4943 | 4943 | 10433 |
| siRNA 4945 | 4945 | 10435 |
| siRNA 4947 | 4947 | 10437 |
| siRNA 4975 | 4975 | 10465 |
| siRNA 4976 | 4976 | 10466 |
| siRNA 4977 | 4977 | 10467 |
| siRNA 4981 | 4981 | 10471 |
| siRNA 4982 | 4982 | 10472 |
| siRNA 4983 | 4983 | 10473 |
| siRNA 4984 | 4984 | 10474 |
| siRNA 4989 | 4989 | 10479 |
| siRNA 4990 | 4990 | 10480 |
| siRNA 4991 | 4991 | 10481 |
| siRNA 4992 | 4992 | 10482 |
| siRNA 4993 | 4993 | 10483 |
| siRNA 4994 | 4994 | 10484 |
| siRNA 4996 | 4996 | 10486 |
| siRNA 4997 | 4997 | 10487 |
| siRNA 4999 | 4999 | 10489 |
| siRNA 5000 | 5000 | 10490 |
| siRNA 5001 | 5001 | 10491 |
| siRNA 5002 | 5002 | 10492 |
| siRNA 5004 | 5004 | 10494 |
| siRNA 5014 | 5014 | 10504 |
| siRNA 5017 | 5017 | 10507 |
| siRNA 5020 | 5020 | 10510 |
| siRNA 5027 | 5027 | 10517 |
| siRNA 5063 | 5063 | 10553 |
| siRNA 5071 | 5071 | 10561 |
| siRNA 5072 | 5072 | 10562 |
| siRNA 5073 | 5073 | 10563 |
| siRNA 5092 | 5092 | 10582 |
| siRNA 5097 | 5097 | 10587 |
| siRNA 5100 | 5100 | 10590 |
| siRNA 5103 | 5103 | 10593 |
| siRNA 5106 | 5106 | 10596 |
| siRNA 5109 | 5109 | 10599 |
| siRNA 5118 | 5118 | 10608 |
| siRNA 5119 | 5119 | 10609 |
| siRNA 5120 | 5120 | 10610 |
| siRNA 5124 | 5124 | 10614 |
| siRNA 5126 | 5126 | 10616 |
| siRNA 5152 | 5152 | 10642 |
| siRNA 5153 | 5153 | 10643 |
| siRNA 5157 | 5157 | 10647 |
| siRNA 5158 | 5158 | 10648 |
| siRNA 5162 | 5162 | 10652 |
| siRNA 5164 | 5164 | 10654 |
| siRNA 5166 | 5166 | 10656 |
| siRNA 5177 | 5177 | 10667 |
| siRNA 5182 | 5182 | 10672 |
| siRNA 5184 | 5184 | 10674 |
| siRNA 5186 | 5186 | 10676 |
| siRNA 5189 | 5189 | 10679 |
| siRNA 5190 | 5190 | 10680 |
| siRNA 5191 | 5191 | 10681 |
| siRNA 5193 | 5193 | 10683 |
| siRNA 5194 | 5194 | 10684 |
| siRNA 5206 | 5206 | 10696 |
| siRNA 5207 | 5207 | 10697 |
| siRNA 5208 | 5208 | 10698 |
| siRNA 5210 | 5210 | 10700 |
| siRNA 5211 | 5211 | 10701 |
| siRNA 5214 | 5214 | 10704 |
| siRNA 5216 | 5216 | 10706 |
| siRNA 5217 | 5217 | 10707 |
| siRNA 5218 | 5218 | 10708 |
| siRNA 5274 | 5274 | 10764 |
| siRNA 5280 | 5280 | 10770 |
| siRNA 5291 | 5291 | 10781 |
| siRNA 5297 | 5297 | 10787 |
| siRNA 5305 | 5305 | 10795 |
| siRNA 5306 | 5306 | 10796 |
| siRNA 5310 | 5310 | 10800 |
| siRNA 5313 | 5313 | 10803 |
| siRNA 5334 | 5334 | 10824 |
| siRNA 5335 | 5335 | 10825 |
| siRNA 5337 | 5337 | 10827 |
| siRNA 5338 | 5338 | 10828 |
| siRNA 5339 | 5339 | 10829 |
| siRNA 5340 | 5340 | 10830 |
| siRNA 5341 | 5341 | 10831 |
| siRNA 5343 | 5343 | 10833 |
| siRNA 5344 | 5344 | 10834 |
| siRNA 5346 | 5346 | 10836 |
| siRNA 5349 | 5349 | 10839 |
| siRNA 5350 | 5350 | 10840 |
| siRNA 5352 | 5352 | 10842 |
| siRNA 5363 | 5363 | 10853 |
| siRNA 5369 | 5369 | 10859 |
| siRNA 5374 | 5374 | 10864 |
| siRNA 5375 | 5375 | 10865 |
| siRNA 5385 | 5385 | 10875 |
| siRNA 5392 | 5392 | 10882 |
| siRNA 5393 | 5393 | 10883 |
| siRNA 5396 | 5396 | 10886 |
| siRNA 5400 | 5400 | 10890 |
| siRNA 5404 | 5404 | 10894 |
| siRNA 5405 | 5405 | 10895 |
| siRNA 5408 | 5408 | 10898 |
| siRNA 5409 | 5409 | 10899 |
| siRNA 5410 | 5410 | 10900 |
| siRNA 5415 | 5415 | 10905 |
| siRNA 5423 | 5423 | 10913 |
| siRNA 5427 | 5427 | 10917 |
| siRNA 5431 | 5431 | 10921 |
| siRNA 5435 | 5435 | 10925 |
| siRNA 5443 | 5443 | 10933 |
| siRNA 5449 | 5449 | 10939 |
| siRNA 5450 | 5450 | 10940 |
| siRNA 5455 | 5455 | 10945 |
| siRNA 5476 | 5476 | 10966 |
| siRNA 5478 | 5478 | 10968 |
| siRNA 5482 | 5482 | 10972 |
| siRNA 5484 | 5484 | 10974 |
| siRNA 5485 | 5485 | 10975 |

The siRNAs in subset A had the following characteristics: Cross-reactivity: With 19mer in human SOS2 mRNA; Specificity category: For human: AS2 or better, SS3 or better; and miRNA seeds: AS+SS strand: seed region not conserved in human, mouse, and rat and not present in >4 species; Off-target frequency: ≤30 human off-targets matched with 2 mismatches in antisense strand; and SNPs: siRNA target sites do not harbor SNPs with a MAF≥100 (pos. 2-18).

The siRNA sequences in subset A were selected for more stringent specificity to yield subset B. Subset B includes 1123 siRNAs whose base sequences are shown in Table 16.

TABLE 16
Subset B

| | Sense | Antisense |
|---|---|---|
| siRNA Name | SEQ ID NO: | SEQ ID NO: |
| siRNA 47 | 47 | 5537 |
| siRNA 52 | 52 | 5542 |
| siRNA 53 | 53 | 5543 |
| siRNA 72 | 72 | 5562 |
| siRNA 105 | 105 | 5595 |
| siRNA 106 | 106 | 5596 |
| siRNA 108 | 108 | 5598 |
| siRNA 115 | 115 | 5605 |
| siRNA 129 | 129 | 5619 |
| siRNA 158 | 158 | 5648 |
| siRNA 159 | 159 | 5649 |
| siRNA 160 | 160 | 5650 |
| siRNA 164 | 164 | 5654 |
| siRNA 165 | 165 | 5655 |
| siRNA 200 | 200 | 5690 |
| siRNA 224 | 224 | 5714 |
| siRNA 233 | 233 | 5723 |
| siRNA 234 | 234 | 5724 |
| siRNA 236 | 236 | 5726 |
| siRNA 258 | 258 | 5748 |
| siRNA 266 | 266 | 5756 |
| siRNA 275 | 275 | 5765 |
| siRNA 281 | 281 | 5771 |
| siRNA 282 | 282 | 5772 |
| siRNA 301 | 301 | 5791 |
| siRNA 303 | 303 | 5793 |
| siRNA 305 | 305 | 5795 |
| siRNA 308 | 308 | 5798 |
| siRNA 310 | 310 | 5800 |
| siRNA 313 | 313 | 5803 |
| siRNA 315 | 315 | 5805 |
| siRNA 317 | 317 | 5807 |
| siRNA 323 | 323 | 5813 |
| siRNA 326 | 326 | 5816 |
| siRNA 331 | 331 | 5821 |
| siRNA 332 | 332 | 5822 |
| siRNA 336 | 336 | 5826 |
| siRNA 338 | 338 | 5828 |
| siRNA 341 | 341 | 5831 |
| siRNA 342 | 342 | 5832 |
| siRNA 347 | 347 | 5837 |
| siRNA 350 | 350 | 5840 |
| siRNA 351 | 351 | 5841 |
| siRNA 352 | 352 | 5842 |
| siRNA 353 | 353 | 5843 |
| siRNA 354 | 354 | 5844 |
| siRNA 356 | 356 | 5846 |
| siRNA 357 | 357 | 5847 |
| siRNA 364 | 364 | 5854 |
| siRNA 368 | 368 | 5858 |
| siRNA 370 | 370 | 5860 |
| siRNA 375 | 375 | 5865 |
| siRNA 376 | 376 | 5866 |
| siRNA 381 | 381 | 5871 |
| siRNA 394 | 394 | 5884 |
| siRNA 400 | 400 | 5890 |
| siRNA 403 | 403 | 5893 |
| siRNA 404 | 404 | 5894 |
| siRNA 405 | 405 | 5895 |
| siRNA 415 | 415 | 5905 |
| siRNA 416 | 416 | 5906 |
| siRNA 422 | 422 | 5912 |
| siRNA 424 | 424 | 5914 |
| siRNA 428 | 428 | 5918 |
| siRNA 436 | 436 | 5926 |
| siRNA 457 | 457 | 5947 |
| siRNA 462 | 462 | 5952 |
| siRNA 463 | 463 | 5953 |
| siRNA 464 | 464 | 5954 |
| siRNA 465 | 465 | 5955 |
| siRNA 468 | 468 | 5958 |
| siRNA 482 | 482 | 5972 |
| siRNA 488 | 488 | 5978 |
| siRNA 493 | 493 | 5983 |
| siRNA 494 | 494 | 5984 |
| siRNA 509 | 509 | 5999 |
| siRNA 510 | 510 | 6000 |
| siRNA 514 | 514 | 6004 |
| siRNA 523 | 523 | 6013 |
| siRNA 526 | 526 | 6016 |
| siRNA 528 | 528 | 6018 |
| siRNA 536 | 536 | 6026 |
| siRNA 538 | 538 | 6028 |
| siRNA 539 | 539 | 6029 |
| siRNA 541 | 541 | 6031 |
| siRNA 542 | 542 | 6032 |
| siRNA 546 | 546 | 6036 |
| siRNA 547 | 547 | 6037 |
| siRNA 552 | 552 | 6042 |
| siRNA 556 | 556 | 6046 |
| siRNA 558 | 558 | 6048 |
| siRNA 562 | 562 | 6052 |
| siRNA 570 | 570 | 6060 |
| siRNA 573 | 573 | 6063 |
| siRNA 574 | 574 | 6064 |
| siRNA 575 | 575 | 6065 |
| siRNA 585 | 585 | 6075 |
| siRNA 587 | 587 | 6077 |
| siRNA 589 | 589 | 6079 |
| siRNA 596 | 596 | 6086 |
| siRNA 602 | 602 | 6092 |
| siRNA 614 | 614 | 6104 |
| siRNA 617 | 617 | 6107 |
| siRNA 619 | 619 | 6109 |
| siRNA 620 | 620 | 6110 |
| siRNA 621 | 621 | 6111 |
| siRNA 622 | 622 | 6112 |
| siRNA 623 | 623 | 6113 |
| siRNA 625 | 625 | 6115 |
| siRNA 627 | 627 | 6117 |
| siRNA 632 | 632 | 6122 |
| siRNA 633 | 633 | 6123 |
| siRNA 637 | 637 | 6127 |
| siRNA 638 | 638 | 6128 |
| siRNA 639 | 639 | 6129 |
| siRNA 640 | 640 | 6130 |
| siRNA 643 | 643 | 6133 |
| siRNA 644 | 644 | 6134 |
| siRNA 647 | 647 | 6137 |
| siRNA 650 | 650 | 6140 |
| siRNA 651 | 651 | 6141 |
| siRNA 652 | 652 | 6142 |
| siRNA 653 | 653 | 6143 |
| siRNA 656 | 656 | 6146 |
| siRNA 657 | 657 | 6147 |
| siRNA 660 | 660 | 6150 |
| siRNA 662 | 662 | 6152 |
| siRNA 663 | 663 | 6153 |
| siRNA 664 | 664 | 6154 |
| siRNA 665 | 665 | 6155 |
| siRNA 666 | 666 | 6156 |
| siRNA 671 | 671 | 6161 |
| siRNA 674 | 674 | 6164 |
| siRNA 678 | 678 | 6168 |
| siRNA 679 | 679 | 6169 |
| siRNA 680 | 680 | 6170 |
| siRNA 682 | 682 | 6172 |
| siRNA 683 | 683 | 6173 |
| siRNA 685 | 685 | 6175 |
| siRNA 689 | 689 | 6179 |
| siRNA 690 | 690 | 6180 |
| siRNA 691 | 691 | 6181 |
| siRNA 692 | 692 | 6182 |
| siRNA 719 | 719 | 6209 |
| siRNA 736 | 736 | 6226 |
| siRNA 737 | 737 | 6227 |
| siRNA 738 | 738 | 6228 |
| siRNA 739 | 739 | 6229 |
| siRNA 740 | 740 | 6230 |
| siRNA 743 | 743 | 6233 |
| siRNA 744 | 744 | 6234 |
| siRNA 749 | 749 | 6239 |
| siRNA 750 | 750 | 6240 |
| siRNA 763 | 763 | 6253 |
| siRNA 764 | 764 | 6254 |
| siRNA 766 | 766 | 6256 |
| siRNA 771 | 771 | 6261 |
| siRNA 772 | 772 | 6262 |
| siRNA 782 | 782 | 6272 |
| siRNA 783 | 783 | 6273 |
| siRNA 784 | 784 | 6274 |
| siRNA 785 | 785 | 6275 |
| siRNA 786 | 786 | 6276 |
| siRNA 787 | 787 | 6277 |
| siRNA 788 | 788 | 6278 |
| siRNA 789 | 789 | 6279 |
| siRNA 792 | 792 | 6282 |
| siRNA 795 | 795 | 6285 |
| siRNA 796 | 796 | 6286 |
| siRNA 797 | 797 | 6287 |
| siRNA 810 | 810 | 6300 |
| siRNA 813 | 813 | 6303 |
| siRNA 818 | 818 | 6308 |
| siRNA 819 | 819 | 6309 |
| siRNA 831 | 831 | 6321 |
| siRNA 853 | 853 | 6343 |
| siRNA 854 | 854 | 6344 |
| siRNA 856 | 856 | 6346 |
| siRNA 857 | 857 | 6347 |
| siRNA 858 | 858 | 6348 |
| siRNA 859 | 859 | 6349 |
| siRNA 872 | 872 | 6362 |
| siRNA 876 | 876 | 6366 |
| siRNA 877 | 877 | 6367 |
| siRNA 884 | 884 | 6374 |
| siRNA 887 | 887 | 6377 |
| siRNA 888 | 888 | 6378 |
| siRNA 889 | 889 | 6379 |
| siRNA 890 | 890 | 6380 |
| siRNA 894 | 894 | 6384 |
| siRNA 922 | 922 | 6412 |
| siRNA 924 | 924 | 6414 |
| siRNA 925 | 925 | 6415 |
| siRNA 926 | 926 | 6416 |
| siRNA 928 | 928 | 6418 |
| siRNA 930 | 930 | 6420 |
| siRNA 933 | 933 | 6423 |
| siRNA 934 | 934 | 6424 |
| siRNA 935 | 935 | 6425 |
| siRNA 936 | 936 | 6426 |
| siRNA 938 | 938 | 6428 |
| siRNA 941 | 941 | 6431 |
| siRNA 942 | 942 | 6432 |
| siRNA 953 | 953 | 6443 |
| siRNA 954 | 954 | 6444 |
| siRNA 959 | 959 | 6449 |
| siRNA 961 | 961 | 6451 |
| siRNA 967 | 967 | 6457 |
| siRNA 973 | 973 | 6463 |
| siRNA 981 | 981 | 6471 |
| siRNA 999 | 999 | 6489 |
| siRNA 1000 | 1000 | 6490 |
| siRNA 1001 | 1001 | 6491 |
| siRNA 1004 | 1004 | 6494 |
| siRNA 1006 | 1006 | 6496 |
| siRNA 1007 | 1007 | 6497 |
| siRNA 1036 | 1036 | 6526 |
| siRNA 1040 | 1040 | 6530 |
| siRNA 1050 | 1050 | 6540 |
| siRNA 1052 | 1052 | 6542 |
| siRNA 1053 | 1053 | 6543 |
| siRNA 1054 | 1054 | 6544 |
| siRNA 1056 | 1056 | 6546 |
| siRNA 1062 | 1062 | 6552 |
| siRNA 1067 | 1067 | 6557 |
| siRNA 1072 | 1072 | 6562 |
| siRNA 1075 | 1075 | 6565 |
| siRNA 1082 | 1082 | 6572 |
| siRNA 1084 | 1084 | 6574 |
| siRNA 1085 | 1085 | 6575 |
| siRNA 1086 | 1086 | 6576 |
| siRNA 1087 | 1087 | 6577 |
| siRNA 1134 | 1134 | 6624 |
| siRNA 1153 | 1153 | 6643 |
| siRNA 1154 | 1154 | 6644 |
| siRNA 1155 | 1155 | 6645 |
| siRNA 1156 | 1156 | 6646 |
| siRNA 1157 | 1157 | 6647 |
| siRNA 1158 | 1158 | 6648 |
| siRNA 1159 | 1159 | 6649 |
| siRNA 1175 | 1175 | 6665 |
| siRNA 1178 | 1178 | 6668 |
| siRNA 1185 | 1185 | 6675 |
| siRNA 1187 | 1187 | 6677 |
| siRNA 1191 | 1191 | 6681 |
| siRNA 1192 | 1192 | 6682 |
| siRNA 1196 | 1196 | 6686 |
| siRNA 1224 | 1224 | 6714 |
| siRNA 1242 | 1242 | 6732 |
| siRNA 1248 | 1248 | 6738 |
| siRNA 1249 | 1249 | 6739 |
| siRNA 1250 | 1250 | 6740 |
| siRNA 1251 | 1251 | 6741 |
| siRNA 1255 | 1255 | 6745 |
| siRNA 1279 | 1279 | 6769 |
| siRNA 1280 | 1280 | 6770 |
| siRNA 1282 | 1282 | 6772 |
| siRNA 1286 | 1286 | 6776 |
| siRNA 1288 | 1288 | 6778 |
| siRNA 1294 | 1294 | 6784 |
| siRNA 1296 | 1296 | 6786 |
| siRNA 1301 | 1301 | 6791 |
| siRNA 1302 | 1302 | 6792 |
| siRNA 1303 | 1303 | 6793 |
| siRNA 1304 | 1304 | 6794 |
| siRNA 1307 | 1307 | 6797 |
| siRNA 1309 | 1309 | 6799 |
| siRNA 1318 | 1318 | 6808 |
| siRNA 1319 | 1319 | 6809 |
| siRNA 1320 | 1320 | 6810 |
| siRNA 1321 | 1321 | 6811 |
| siRNA 1322 | 1322 | 6812 |
| siRNA 1326 | 1326 | 6816 |
| siRNA 1333 | 1333 | 6823 |
| siRNA 1339 | 1339 | 6829 |
| siRNA 1340 | 1340 | 6830 |
| siRNA 1342 | 1342 | 6832 |
| siRNA 1343 | 1343 | 6833 |
| siRNA 1344 | 1344 | 6834 |
| siRNA 1346 | 1346 | 6836 |
| siRNA 1347 | 1347 | 6837 |
| siRNA 1351 | 1351 | 6841 |
| siRNA 1357 | 1357 | 6847 |
| siRNA 1363 | 1363 | 6853 |
| siRNA 1365 | 1365 | 6855 |
| siRNA 1372 | 1372 | 6862 |
| siRNA 1395 | 1395 | 6885 |
| siRNA 1398 | 1398 | 6888 |
| siRNA 1405 | 1405 | 6895 |
| siRNA 1406 | 1406 | 6896 |
| siRNA 1408 | 1408 | 6898 |
| siRNA 1410 | 1410 | 6900 |
| siRNA 1411 | 1411 | 6901 |
| siRNA 1422 | 1422 | 6912 |
| siRNA 1430 | 1430 | 6920 |
| siRNA 1431 | 1431 | 6921 |
| siRNA 1434 | 1434 | 6924 |
| siRNA 1435 | 1435 | 6925 |
| siRNA 1441 | 1441 | 6931 |
| siRNA 1442 | 1442 | 6932 |
| siRNA 1445 | 1445 | 6935 |
| siRNA 1448 | 1448 | 6938 |
| siRNA 1449 | 1449 | 6939 |
| siRNA 1452 | 1452 | 6942 |
| siRNA 1453 | 1453 | 6943 |
| siRNA 1459 | 1459 | 6949 |
| siRNA 1460 | 1460 | 6950 |
| siRNA 1461 | 1461 | 6951 |
| siRNA 1462 | 1462 | 6952 |
| siRNA 1463 | 1463 | 6953 |
| siRNA 1465 | 1465 | 6955 |
| siRNA 1466 | 1466 | 6956 |
| siRNA 1467 | 1467 | 6957 |
| siRNA 1468 | 1468 | 6958 |
| siRNA 1469 | 1469 | 6959 |
| siRNA 1470 | 1470 | 6960 |
| siRNA 1472 | 1472 | 6962 |
| siRNA 1473 | 1473 | 6963 |
| siRNA 1474 | 1474 | 6964 |
| siRNA 1475 | 1475 | 6965 |
| siRNA 1476 | 1476 | 6966 |
| siRNA 1477 | 1477 | 6967 |
| siRNA 1481 | 1481 | 6971 |
| siRNA 1494 | 1494 | 6984 |
| siRNA 1495 | 1495 | 6985 |
| siRNA 1498 | 1498 | 6988 |
| siRNA 1503 | 1503 | 6993 |
| siRNA 1504 | 1504 | 6994 |
| siRNA 1505 | 1505 | 6995 |
| siRNA 1510 | 1510 | 7000 |
| siRNA 1521 | 1521 | 7011 |
| siRNA 1529 | 1529 | 7019 |
| siRNA 1533 | 1533 | 7023 |
| siRNA 1535 | 1535 | 7025 |
| siRNA 1560 | 1560 | 7050 |
| siRNA 1568 | 1568 | 7058 |
| siRNA 1569 | 1569 | 7059 |
| siRNA 1570 | 1570 | 7060 |
| siRNA 1572 | 1572 | 7062 |
| siRNA 1575 | 1575 | 7065 |
| siRNA 1587 | 1587 | 7077 |
| siRNA 1588 | 1588 | 7078 |
| siRNA 1589 | 1589 | 7079 |
| siRNA 1596 | 1596 | 7086 |
| siRNA 1614 | 1614 | 7104 |
| siRNA 1621 | 1621 | 7111 |
| siRNA 1622 | 1622 | 7112 |
| siRNA 1623 | 1623 | 7113 |
| siRNA 1627 | 1627 | 7117 |
| siRNA 1628 | 1628 | 7118 |
| siRNA 1631 | 1631 | 7121 |
| siRNA 1632 | 1632 | 7122 |
| siRNA 1633 | 1633 | 7123 |
| siRNA 1634 | 1634 | 7124 |
| siRNA 1635 | 1635 | 7125 |
| siRNA 1636 | 1636 | 7126 |
| siRNA 1637 | 1637 | 7127 |
| siRNA 1638 | 1638 | 7128 |
| siRNA 1641 | 1641 | 7131 |
| siRNA 1643 | 1643 | 7133 |
| siRNA 1644 | 1644 | 7134 |
| siRNA 1645 | 1645 | 7135 |
| siRNA 1648 | 1648 | 7138 |
| siRNA 1649 | 1649 | 7139 |
| siRNA 1650 | 1650 | 7140 |
| siRNA 1651 | 1651 | 7141 |
| siRNA 1652 | 1652 | 7142 |
| siRNA 1654 | 1654 | 7144 |
| siRNA 1655 | 1655 | 7145 |
| siRNA 1656 | 1656 | 7146 |
| siRNA 1658 | 1658 | 7148 |
| siRNA 1659 | 1659 | 7149 |
| siRNA 1683 | 1683 | 7173 |
| siRNA 1684 | 1684 | 7174 |
| siRNA 1689 | 1689 | 7179 |
| siRNA 1691 | 1691 | 7181 |
| siRNA 1693 | 1693 | 7183 |
| siRNA 1697 | 1697 | 7187 |
| siRNA 1699 | 1699 | 7189 |
| siRNA 1703 | 1703 | 7193 |
| siRNA 1706 | 1706 | 7196 |
| siRNA 1707 | 1707 | 7197 |
| siRNA 1708 | 1708 | 7198 |
| siRNA 1712 | 1712 | 7202 |
| siRNA 1720 | 1720 | 7210 |
| siRNA 1763 | 1763 | 7253 |
| siRNA 1802 | 1802 | 7292 |
| siRNA 1805 | 1805 | 7295 |
| siRNA 1806 | 1806 | 7296 |
| siRNA 1811 | 1811 | 7301 |
| siRNA 1812 | 1812 | 7302 |
| siRNA 1814 | 1814 | 7304 |
| siRNA 1816 | 1816 | 7306 |
| siRNA 1821 | 1821 | 7311 |
| siRNA 1825 | 1825 | 7315 |
| siRNA 1826 | 1826 | 7316 |
| siRNA 1832 | 1832 | 7322 |
| siRNA 1834 | 1834 | 7324 |
| siRNA 1844 | 1844 | 7334 |
| siRNA 1846 | 1846 | 7336 |
| siRNA 1847 | 1847 | 7337 |
| siRNA 1851 | 1851 | 7341 |
| siRNA 1857 | 1857 | 7347 |
| siRNA 1861 | 1861 | 7351 |
| siRNA 1865 | 1865 | 7355 |
| siRNA 1897 | 1897 | 7387 |
| siRNA 1909 | 1909 | 7399 |
| siRNA 1912 | 1912 | 7402 |
| siRNA 1914 | 1914 | 7404 |
| siRNA 1915 | 1915 | 7405 |
| siRNA 1916 | 1916 | 7406 |
| siRNA 1917 | 1917 | 7407 |
| siRNA 1918 | 1918 | 7408 |
| siRNA 1919 | 1919 | 7409 |
| siRNA 1920 | 1920 | 7410 |
| siRNA 1921 | 1921 | 7411 |
| siRNA 1922 | 1922 | 7412 |
| siRNA 1923 | 1923 | 7413 |
| siRNA 1924 | 1924 | 7414 |
| siRNA 1926 | 1926 | 7416 |
| siRNA 1927 | 1927 | 7417 |
| siRNA 1928 | 1928 | 7418 |
| siRNA 1929 | 1929 | 7419 |
| siRNA 1934 | 1934 | 7424 |
| siRNA 1935 | 1935 | 7425 |
| siRNA 1936 | 1936 | 7426 |
| siRNA 1937 | 1937 | 7427 |
| siRNA 1938 | 1938 | 7428 |
| siRNA 1940 | 1940 | 7430 |
| siRNA 1941 | 1941 | 7431 |
| siRNA 1948 | 1948 | 7438 |
| siRNA 1951 | 1951 | 7441 |
| siRNA 1977 | 1977 | 7467 |
| siRNA 1980 | 1980 | 7470 |
| siRNA 1981 | 1981 | 7471 |
| siRNA 1984 | 1984 | 7474 |
| siRNA 1985 | 1985 | 7475 |
| siRNA 1986 | 1986 | 7476 |
| siRNA 1987 | 1987 | 7477 |
| siRNA 1988 | 1988 | 7478 |
| siRNA 1989 | 1989 | 7479 |
| siRNA 1991 | 1991 | 7481 |
| siRNA 1992 | 1992 | 7482 |
| siRNA 1994 | 1994 | 7484 |
| siRNA 1995 | 1995 | 7485 |
| siRNA 1996 | 1996 | 7486 |
| siRNA 1999 | 1999 | 7489 |
| siRNA 2000 | 2000 | 7490 |
| siRNA 2002 | 2002 | 7492 |
| siRNA 2005 | 2005 | 7495 |
| siRNA 2006 | 2006 | 7496 |
| siRNA 2007 | 2007 | 7497 |
| siRNA 2014 | 2014 | 7504 |
| siRNA 2020 | 2020 | 7510 |
| siRNA 2049 | 2049 | 7539 |
| siRNA 2051 | 2051 | 7541 |
| siRNA 2056 | 2056 | 7546 |
| siRNA 2057 | 2057 | 7547 |
| siRNA 2062 | 2062 | 7552 |
| siRNA 2065 | 2065 | 7555 |
| siRNA 2071 | 2071 | 7561 |
| siRNA 2073 | 2073 | 7563 |
| siRNA 2074 | 2074 | 7564 |
| siRNA 2077 | 2077 | 7567 |
| siRNA 2088 | 2088 | 7578 |
| siRNA 2096 | 2096 | 7586 |
| siRNA 2109 | 2109 | 7599 |
| siRNA 2112 | 2112 | 7602 |
| siRNA 2116 | 2116 | 7606 |
| siRNA 2120 | 2120 | 7610 |
| siRNA 2142 | 2142 | 7632 |
| siRNA 2147 | 2147 | 7637 |
| siRNA 2148 | 2148 | 7638 |
| siRNA 2149 | 2149 | 7639 |
| siRNA 2150 | 2150 | 7640 |
| siRNA 2151 | 2151 | 7641 |
| siRNA 2152 | 2152 | 7642 |
| siRNA 2154 | 2154 | 7644 |
| siRNA 2157 | 2157 | 7647 |
| siRNA 2158 | 2158 | 7648 |
| siRNA 2161 | 2161 | 7651 |
| siRNA 2164 | 2164 | 7654 |
| siRNA 2167 | 2167 | 7657 |
| siRNA 2168 | 2168 | 7658 |
| siRNA 2170 | 2170 | 7660 |
| siRNA 2171 | 2171 | 7661 |
| siRNA 2172 | 2172 | 7662 |
| siRNA 2175 | 2175 | 7665 |
| siRNA 2193 | 2193 | 7683 |
| siRNA 2202 | 2202 | 7692 |
| siRNA 2209 | 2209 | 7699 |
| siRNA 2213 | 2213 | 7703 |
| siRNA 2214 | 2214 | 7704 |
| siRNA 2216 | 2216 | 7706 |
| siRNA 2219 | 2219 | 7709 |
| siRNA 2220 | 2220 | 7710 |
| siRNA 2244 | 2244 | 7734 |
| siRNA 2245 | 2245 | 7735 |
| siRNA 2246 | 2246 | 7736 |
| siRNA 2250 | 2250 | 7740 |
| siRNA 2251 | 2251 | 7741 |
| siRNA 2252 | 2252 | 7742 |
| siRNA 2254 | 2254 | 7744 |
| siRNA 2257 | 2257 | 7747 |
| siRNA 2258 | 2258 | 7748 |
| siRNA 2259 | 2259 | 7749 |
| siRNA 2286 | 2286 | 7776 |
| siRNA 2287 | 2287 | 7777 |
| siRNA 2292 | 2292 | 7782 |
| siRNA 2294 | 2294 | 7784 |
| siRNA 2306 | 2306 | 7796 |
| siRNA 2307 | 2307 | 7797 |
| siRNA 2310 | 2310 | 7800 |
| siRNA 2311 | 2311 | 7801 |
| siRNA 2312 | 2312 | 7802 |
| siRNA 2313 | 2313 | 7803 |
| siRNA 2314 | 2314 | 7804 |
| siRNA 2318 | 2318 | 7808 |
| siRNA 2322 | 2322 | 7812 |
| siRNA 2324 | 2324 | 7814 |
| siRNA 2328 | 2328 | 7818 |
| siRNA 2329 | 2329 | 7819 |
| siRNA 2332 | 2332 | 7822 |
| siRNA 2336 | 2336 | 7826 |
| siRNA 2338 | 2338 | 7828 |
| siRNA 2339 | 2339 | 7829 |
| siRNA 2340 | 2340 | 7830 |
| siRNA 2355 | 2355 | 7845 |
| siRNA 2356 | 2356 | 7846 |
| siRNA 2357 | 2357 | 7847 |
| siRNA 2360 | 2360 | 7850 |
| siRNA 2362 | 2362 | 7852 |
| siRNA 2364 | 2364 | 7854 |
| siRNA 2365 | 2365 | 7855 |
| siRNA 2366 | 2366 | 7856 |
| siRNA 2368 | 2368 | 7858 |
| siRNA 2369 | 2369 | 7859 |
| siRNA 2370 | 2370 | 7860 |
| siRNA 2393 | 2393 | 7883 |
| siRNA 2403 | 2403 | 7893 |
| siRNA 2417 | 2417 | 7907 |
| siRNA 2418 | 2418 | 7908 |
| siRNA 2436 | 2436 | 7926 |
| siRNA 2437 | 2437 | 1927 |
| siRNA 2467 | 2467 | 7957 |
| siRNA 2470 | 2470 | 7960 |
| siRNA 2471 | 2471 | 7961 |
| siRNA 2472 | 2472 | 7962 |
| siRNA 2477 | 2477 | 7967 |
| siRNA 2480 | 2480 | 7970 |
| siRNA 2483 | 2483 | 7973 |
| siRNA 2486 | 2486 | 7976 |
| siRNA 2488 | 2488 | 7978 |
| siRNA 2489 | 2489 | 7979 |
| siRNA 2490 | 2490 | 7980 |
| siRNA 2528 | 2528 | 8018 |
| siRNA 2530 | 2530 | 8020 |
| siRNA 2531 | 2531 | 8021 |
| siRNA 2532 | 2532 | 8022 |
| siRNA 2548 | 2548 | 8038 |
| siRNA 2559 | 2559 | 8049 |
| siRNA 2565 | 2565 | 8055 |
| siRNA 2568 | 2568 | 8058 |
| siRNA 2575 | 2575 | 8065 |
| siRNA 2577 | 2577 | 8067 |
| siRNA 2598 | 2598 | 8088 |
| siRNA 2610 | 2610 | 8100 |
| siRNA 2611 | 2611 | 8101 |
| siRNA 2616 | 2616 | 8106 |
| siRNA 2619 | 2619 | 8109 |
| siRNA 2620 | 2620 | 8110 |
| siRNA 2623 | 2623 | 8113 |
| siRNA 2630 | 2630 | 8120 |
| siRNA 2631 | 2631 | 8121 |
| siRNA 2632 | 2632 | 8122 |
| siRNA 2633 | 2633 | 8123 |
| siRNA 2637 | 2637 | 8127 |
| siRNA 2640 | 2640 | 8130 |
| siRNA 2642 | 2642 | 8132 |
| siRNA 2644 | 2644 | 8134 |
| siRNA 2645 | 2645 | 8135 |
| siRNA 2646 | 2646 | 8136 |
| siRNA 2673 | 2673 | 8163 |
| siRNA 2675 | 2675 | 8165 |
| siRNA 2679 | 2679 | 8169 |
| siRNA 2680 | 2680 | 8170 |
| siRNA 2682 | 2682 | 8172 |
| siRNA 2683 | 2683 | 8173 |
| siRNA 2688 | 2688 | 8178 |
| siRNA 2690 | 2690 | 8180 |
| siRNA 2691 | 2691 | 8181 |
| siRNA 2699 | 2699 | 8189 |
| siRNA 2708 | 2708 | 8198 |
| siRNA 2713 | 2713 | 8203 |
| siRNA 2755 | 2755 | 8245 |
| siRNA 2756 | 2756 | 8246 |
| siRNA 2757 | 2757 | 8247 |
| siRNA 2759 | 2759 | 8249 |
| siRNA 2760 | 2760 | 8250 |
| siRNA 2761 | 2761 | 8251 |
| siRNA 2762 | 2762 | 8252 |
| siRNA 2763 | 2763 | 8253 |
| siRNA 2764 | 2764 | 8254 |
| siRNA 2767 | 2767 | 8257 |
| siRNA 2815 | 2815 | 8305 |
| siRNA 2816 | 2816 | 8306 |
| siRNA 2817 | 2817 | 8307 |
| siRNA 2821 | 2821 | 8311 |
| siRNA 2822 | 2822 | 8312 |
| siRNA 2825 | 2825 | 8315 |
| siRNA 2828 | 2828 | 8318 |
| siRNA 2829 | 2829 | 8319 |
| siRNA 2830 | 2830 | 8320 |
| siRNA 2833 | 2833 | 8323 |
| siRNA 2834 | 2834 | 8324 |
| siRNA 2836 | 2836 | 8326 |
| siRNA 2841 | 2841 | 8331 |
| siRNA 2842 | 2842 | 8332 |
| siRNA 2847 | 2847 | 8337 |
| siRNA 2866 | 2866 | 8356 |
| siRNA 2882 | 2882 | 8372 |
| siRNA 2883 | 2883 | 8373 |
| siRNA 2884 | 2884 | 8374 |
| siRNA 2886 | 2886 | 8376 |
| siRNA 2887 | 2887 | 8377 |
| siRNA 2888 | 2888 | 8378 |
| siRNA 2890 | 2890 | 8380 |
| siRNA 2897 | 2897 | 8387 |
| siRNA 2898 | 2898 | 8388 |
| siRNA 2902 | 2902 | 8392 |
| siRNA 2903 | 2903 | 8393 |
| siRNA 2908 | 2908 | 8398 |
| siRNA 2917 | 2917 | 8407 |
| siRNA 2926 | 2926 | 8416 |
| siRNA 2928 | 2928 | 8418 |
| siRNA 2929 | 2929 | 8419 |
| siRNA 2930 | 2930 | 8420 |
| siRNA 2932 | 2932 | 8422 |
| siRNA 2933 | 2933 | 8423 |
| siRNA 2937 | 2937 | 8427 |
| siRNA 2938 | 2938 | 8428 |
| siRNA 2939 | 2939 | 8429 |
| siRNA 2940 | 2940 | 8430 |
| siRNA 2942 | 2942 | 8432 |
| siRNA 2944 | 2944 | 8434 |
| siRNA 2946 | 2946 | 8436 |
| siRNA 2947 | 2947 | 8437 |
| siRNA 2949 | 2949 | 8439 |
| siRNA 2952 | 2952 | 8442 |
| siRNA 2981 | 2981 | 8471 |
| siRNA 2982 | 2982 | 8472 |
| siRNA 2983 | 2983 | 8473 |
| siRNA 2984 | 2984 | 8474 |
| siRNA 2985 | 2985 | 8475 |
| siRNA 2986 | 2986 | 8476 |
| siRNA 2987 | 2987 | 8477 |
| siRNA 2998 | 2998 | 8488 |
| siRNA 3006 | 3006 | 8496 |
| siRNA 3024 | 3024 | 8514 |
| siRNA 3025 | 3025 | 8515 |
| siRNA 3035 | 3035 | 8525 |
| siRNA 3038 | 3038 | 8528 |
| siRNA 3042 | 3042 | 8532 |
| siRNA 3046 | 3046 | 8536 |
| siRNA 3053 | 3053 | 8543 |
| siRNA 3091 | 3091 | 8581 |
| siRNA 3093 | 3093 | 8583 |
| siRNA 3094 | 3094 | 8584 |
| siRNA 3098 | 3098 | 8588 |
| siRNA 3099 | 3099 | 8589 |
| siRNA 3108 | 3108 | 8598 |
| siRNA 3140 | 3140 | 8630 |
| siRNA 3146 | 3146 | 8636 |
| siRNA 3156 | 3156 | 8646 |
| siRNA 3164 | 3164 | 8654 |
| siRNA 3200 | 3200 | 8690 |
| siRNA 3209 | 3209 | 8699 |
| siRNA 3218 | 3218 | 8708 |
| siRNA 3221 | 3221 | 8711 |
| siRNA 3222 | 3222 | 8712 |
| siRNA 3223 | 3223 | 8713 |
| siRNA 3224 | 3224 | 8714 |
| siRNA 3230 | 3230 | 8720 |
| siRNA 3231 | 3231 | 8721 |
| siRNA 3232 | 3232 | 8722 |
| siRNA 3238 | 3238 | 8728 |
| siRNA 3247 | 3247 | 8737 |
| siRNA 3259 | 3259 | 8749 |
| siRNA 3261 | 3261 | 8751 |
| siRNA 3262 | 3262 | 8752 |
| siRNA 3263 | 3263 | 8753 |
| siRNA 3276 | 3276 | 8766 |
| siRNA 3277 | 3277 | 8767 |
| siRNA 3305 | 3305 | 8795 |
| siRNA 3310 | 3310 | 8800 |
| siRNA 3315 | 3315 | 8805 |
| siRNA 3330 | 3330 | 8820 |
| siRNA 3333 | 3333 | 8823 |
| siRNA 3334 | 3334 | 8824 |
| siRNA 3335 | 3335 | 8825 |
| siRNA 3339 | 3339 | 8829 |
| siRNA 3342 | 3342 | 8832 |
| siRNA 3349 | 3349 | 8839 |
| siRNA 3350 | 3350 | 8840 |
| siRNA 3351 | 3351 | 8841 |
| siRNA 3352 | 3352 | 8842 |
| siRNA 3353 | 3353 | 8843 |
| siRNA 3354 | 3354 | 8844 |
| siRNA 3355 | 3355 | 8845 |
| siRNA 3357 | 3357 | 8847 |
| siRNA 3358 | 3358 | 8848 |
| siRNA 3359 | 3359 | 8849 |
| siRNA 3360 | 3360 | 8850 |
| siRNA 3364 | 3364 | 8854 |
| siRNA 3396 | 3396 | 8886 |
| siRNA 3397 | 3397 | 8887 |
| siRNA 3403 | 3403 | 8893 |
| siRNA 3405 | 3405 | 8895 |
| siRNA 3406 | 3406 | 8896 |
| siRNA 3407 | 3407 | 8897 |
| siRNA 3410 | 3410 | 8900 |
| siRNA 3411 | 3411 | 8901 |
| siRNA 3412 | 3412 | 8902 |
| siRNA 3413 | 3413 | 8903 |
| siRNA 3415 | 3415 | 8905 |
| siRNA 3416 | 3416 | 8906 |
| siRNA 3417 | 3417 | 8907 |
| siRNA 3418 | 3418 | 8908 |
| siRNA 3425 | 3425 | 8915 |
| siRNA 3431 | 3431 | 8921 |
| siRNA 3432 | 3432 | 8922 |
| siRNA 3435 | 3435 | 8925 |
| siRNA 3436 | 3436 | 8926 |
| siRNA 3437 | 3437 | 8927 |
| siRNA 3438 | 3438 | 8928 |
| siRNA 3439 | 3439 | 8929 |
| siRNA 3443 | 3443 | 8933 |
| siRNA 3444 | 3444 | 8934 |
| siRNA 3449 | 3449 | 8939 |
| siRNA 3450 | 3450 | 8940 |
| siRNA 3451 | 3451 | 8941 |
| siRNA 3456 | 3456 | 8946 |
| siRNA 3457 | 3457 | 8947 |
| siRNA 3458 | 3458 | 8948 |
| siRNA 3460 | 3460 | 8950 |
| siRNA 3461 | 3461 | 8951 |
| siRNA 3470 | 3470 | 8960 |
| siRNA 3471 | 3471 | 8961 |
| siRNA 3472 | 3472 | 8962 |
| siRNA 3484 | 3484 | 8974 |
| siRNA 3487 | 3487 | 8977 |
| siRNA 3488 | 3488 | 8978 |
| siRNA 3489 | 3489 | 8979 |
| siRNA 3490 | 3490 | 8980 |
| siRNA 3491 | 3491 | 8981 |
| siRNA 3492 | 3492 | 8982 |
| siRNA 3494 | 3494 | 8984 |
| siRNA 3495 | 3495 | 8985 |
| siRNA 3500 | 3500 | 8990 |
| siRNA 3502 | 3502 | 8992 |
| siRNA 3504 | 3504 | 8994 |
| siRNA 3511 | 3511 | 9001 |
| siRNA 3512 | 3512 | 9002 |
| siRNA 3516 | 3516 | 9006 |
| siRNA 3517 | 3517 | 9007 |
| siRNA 3540 | 3540 | 9030 |
| siRNA 3546 | 3546 | 9036 |
| siRNA 3549 | 3549 | 9039 |
| siRNA 3558 | 3558 | 9048 |
| siRNA 3560 | 3560 | 9050 |
| siRNA 3561 | 3561 | 9051 |
| siRNA 3564 | 3564 | 9054 |
| siRNA 3566 | 3566 | 9056 |
| siRNA 3576 | 3576 | 9066 |
| siRNA 3582 | 3582 | 9072 |
| siRNA 3583 | 3583 | 9073 |
| siRNA 3585 | 3585 | 9075 |
| siRNA 3586 | 3586 | 9076 |
| siRNA 3587 | 3587 | 9077 |
| siRNA 3602 | 3602 | 9092 |
| siRNA 3604 | 3604 | 9094 |
| siRNA 3605 | 3605 | 9095 |
| siRNA 3610 | 3610 | 9100 |
| siRNA 3619 | 3619 | 9109 |
| siRNA 3629 | 3629 | 9119 |
| siRNA 3634 | 3634 | 9124 |
| siRNA 3635 | 3635 | 9125 |
| siRNA 3637 | 3637 | 9127 |
| siRNA 3638 | 3638 | 9128 |
| siRNA 3639 | 3639 | 9129 |
| siRNA 3640 | 3640 | 9130 |
| siRNA 3641 | 3641 | 9131 |
| siRNA 3663 | 3663 | 9153 |
| siRNA 3677 | 3677 | 9167 |
| siRNA 3685 | 3685 | 9175 |
| siRNA 3686 | 3686 | 9176 |
| siRNA 3688 | 3688 | 9178 |
| siRNA 3691 | 3691 | 9181 |
| siRNA 3692 | 3692 | 9182 |
| siRNA 3695 | 3695 | 9185 |
| siRNA 3703 | 3703 | 9193 |
| siRNA 3709 | 3709 | 9199 |
| siRNA 3712 | 3712 | 9202 |
| siRNA 3746 | 3746 | 9236 |
| siRNA 3747 | 3747 | 9237 |
| siRNA 3748 | 3748 | 9238 |
| siRNA 3756 | 3756 | 9246 |
| siRNA 3792 | 3792 | 9282 |
| siRNA 3802 | 3802 | 9292 |
| siRNA 3803 | 3803 | 9293 |
| siRNA 3807 | 3807 | 9297 |
| siRNA 3810 | 3810 | 9300 |
| siRNA 3816 | 3816 | 9306 |
| siRNA 3838 | 3838 | 9328 |
| siRNA 3840 | 3840 | 9330 |
| siRNA 3841 | 3841 | 9331 |
| siRNA 3868 | 3868 | 9358 |
| siRNA 3869 | 3869 | 9359 |
| siRNA 3874 | 3874 | 9364 |
| siRNA 3875 | 3875 | 9365 |
| siRNA 3883 | 3883 | 9373 |
| siRNA 3897 | 3897 | 9387 |
| siRNA 3908 | 3908 | 9398 |
| siRNA 3909 | 3909 | 9399 |
| siRNA 3911 | 3911 | 9401 |
| siRNA 3912 | 3912 | 9402 |
| siRNA 3922 | 3922 | 9412 |
| siRNA 3926 | 3926 | 9416 |
| siRNA 3934 | 3934 | 9424 |
| siRNA 3936 | 3936 | 9426 |
| siRNA 3943 | 3943 | 9433 |
| siRNA 3944 | 3944 | 9434 |
| siRNA 3945 | 3945 | 9435 |
| siRNA 3960 | 3960 | 9450 |
| siRNA 3969 | 3969 | 9459 |
| siRNA 3970 | 3970 | 9460 |
| siRNA 3988 | 3988 | 9478 |
| siRNA 3991 | 3991 | 9481 |
| siRNA 4010 | 4010 | 9500 |
| siRNA 4025 | 4025 | 9515 |
| siRNA 4027 | 4027 | 9517 |
| siRNA 4036 | 4036 | 9526 |
| siRNA 4038 | 4038 | 9528 |
| siRNA 4040 | 4040 | 9530 |
| siRNA 4041 | 4041 | 9531 |
| siRNA 4043 | 4043 | 9533 |
| siRNA 4044 | 4044 | 9534 |
| siRNA 4047 | 4047 | 9537 |
| siRNA 4048 | 4048 | 9538 |
| siRNA 4049 | 4049 | 9539 |
| siRNA 4050 | 4050 | 9540 |
| siRNA 4051 | 4051 | 9541 |
| siRNA 4052 | 4052 | 9542 |
| siRNA 4053 | 4053 | 9543 |
| siRNA 4054 | 4054 | 9544 |
| siRNA 4056 | 4056 | 9546 |
| siRNA 4058 | 4058 | 9548 |
| siRNA 4064 | 4064 | 9554 |
| siRNA 4068 | 4068 | 9558 |
| siRNA 4070 | 4070 | 9560 |
| siRNA 4077 | 4077 | 9567 |
| siRNA 4083 | 4083 | 9573 |
| siRNA 4111 | 4111 | 9601 |
| siRNA 4112 | 4112 | 9602 |
| siRNA 4117 | 4117 | 9607 |
| siRNA 4122 | 4122 | 9612 |
| siRNA 4125 | 4125 | 9615 |
| siRNA 4132 | 4132 | 9622 |
| siRNA 4133 | 4133 | 9623 |
| siRNA 4138 | 4138 | 9628 |
| siRNA 4169 | 4169 | 9659 |
| siRNA 4179 | 4179 | 9669 |
| siRNA 4190 | 4190 | 9680 |
| siRNA 4192 | 4192 | 9682 |
| siRNA 4199 | 4199 | 9689 |
| siRNA 4207 | 4207 | 9697 |
| siRNA 4209 | 4209 | 9699 |
| siRNA 4210 | 4210 | 9700 |
| siRNA 4211 | 4211 | 9701 |
| siRNA 4212 | 4212 | 9702 |
| siRNA 4213 | 4213 | 9703 |
| siRNA 4214 | 4214 | 9704 |
| siRNA 4216 | 4216 | 9706 |
| siRNA 4218 | 4218 | 9708 |
| siRNA 4219 | 4219 | 9709 |
| siRNA 4222 | 4222 | 9712 |
| siRNA 4223 | 4223 | 9713 |
| siRNA 4224 | 4224 | 9714 |
| siRNA 4225 | 4225 | 9715 |
| siRNA 4226 | 4226 | 9716 |
| siRNA 4228 | 4228 | 9718 |
| siRNA 4233 | 4233 | 9723 |
| siRNA 4235 | 4235 | 9725 |
| siRNA 4237 | 4237 | 9727 |
| siRNA 4238 | 4238 | 9728 |
| siRNA 4242 | 4242 | 9732 |
| siRNA 4243 | 4243 | 9733 |
| siRNA 4245 | 4245 | 9735 |
| siRNA 4247 | 4247 | 9737 |
| siRNA 4252 | 4252 | 9742 |
| siRNA 4254 | 4254 | 9744 |
| siRNA 4275 | 4275 | 9765 |
| siRNA 4276 | 4276 | 9766 |
| siRNA 4277 | 4277 | 9767 |
| siRNA 4279 | 4279 | 9769 |
| siRNA 4282 | 4282 | 9772 |
| siRNA 4283 | 4283 | 9773 |
| siRNA 4284 | 4284 | 9774 |
| siRNA 4290 | 4290 | 9780 |
| siRNA 4292 | 4292 | 9782 |
| siRNA 4293 | 4293 | 9783 |
| siRNA 4294 | 4294 | 9784 |
| siRNA 4295 | 4295 | 9785 |
| siRNA 4296 | 4296 | 9786 |
| siRNA 4297 | 4297 | 9787 |
| siRNA 4305 | 4305 | 9795 |
| siRNA 4309 | 4309 | 9799 |
| siRNA 4313 | 4313 | 9803 |
| siRNA 4319 | 4319 | 9809 |
| siRNA 4322 | 4322 | 9812 |
| siRNA 4351 | 4351 | 9841 |
| siRNA 4356 | 4356 | 9846 |
| siRNA 4368 | 4368 | 9858 |
| siRNA 4382 | 4382 | 9872 |
| siRNA 4388 | 4388 | 9878 |
| siRNA 4409 | 4409 | 9899 |
| siRNA 4414 | 4414 | 9904 |
| siRNA 4453 | 4453 | 9943 |
| siRNA 4455 | 4455 | 9945 |
| siRNA 4456 | 4456 | 9946 |
| siRNA 4459 | 4459 | 9949 |
| siRNA 4460 | 4460 | 9950 |
| siRNA 4467 | 4467 | 9957 |
| siRNA 4468 | 4468 | 9958 |
| siRNA 4474 | 4474 | 9964 |
| siRNA 4475 | 4475 | 9965 |
| siRNA 4481 | 4481 | 9971 |
| siRNA 4482 | 4482 | 9972 |
| siRNA 4487 | 4487 | 9977 |
| siRNA 4488 | 4488 | 9978 |
| siRNA 4489 | 4489 | 9979 |
| siRNA 4490 | 4490 | 9980 |
| siRNA 4491 | 4491 | 9981 |
| siRNA 4492 | 4492 | 9982 |
| siRNA 4496 | 4496 | 9986 |
| siRNA 4518 | 4518 | 10008 |
| siRNA 4519 | 4519 | 10009 |
| siRNA 4521 | 4521 | 10011 |
| siRNA 4527 | 4527 | 10017 |
| siRNA 4545 | 4545 | 10035 |
| siRNA 4547 | 4547 | 10037 |
| siRNA 4551 | 4551 | 10041 |
| siRNA 4552 | 4552 | 10042 |
| siRNA 4561 | 4561 | 10051 |
| siRNA 4565 | 4565 | 10055 |
| siRNA 4576 | 4576 | 10066 |
| siRNA 4584 | 4584 | 10074 |
| siRNA 4593 | 4593 | 10083 |
| siRNA 4594 | 4594 | 10084 |
| siRNA 4595 | 4595 | 10085 |
| siRNA 4596 | 4596 | 10086 |
| siRNA 4597 | 4597 | 10087 |
| siRNA 4598 | 4598 | 10088 |
| siRNA 4599 | 4599 | 10089 |
| siRNA 4600 | 4600 | 10090 |
| siRNA 4601 | 4601 | 10091 |
| siRNA 4628 | 4628 | 10118 |
| siRNA 4629 | 4629 | 10119 |
| siRNA 4644 | 4644 | 10134 |
| siRNA 4674 | 4674 | 10164 |
| siRNA 4675 | 4675 | 10165 |
| siRNA 4679 | 4679 | 10169 |
| siRNA 4680 | 4680 | 10170 |
| siRNA 4682 | 4682 | 10172 |
| siRNA 4705 | 4705 | 10195 |
| siRNA 4726 | 4726 | 10216 |
| siRNA 4739 | 4739 | 10229 |
| siRNA 4742 | 4742 | 10232 |
| siRNA 4749 | 4749 | 10239 |
| siRNA 4754 | 4754 | 10244 |
| siRNA 4756 | 4756 | 10246 |
| siRNA 4758 | 4758 | 10248 |
| siRNA 4763 | 4763 | 10253 |
| siRNA 4765 | 4765 | 10255 |
| siRNA 4767 | 4767 | 10257 |
| siRNA 4770 | 4770 | 10260 |
| siRNA 4788 | 4788 | 10278 |
| siRNA 4797 | 4797 | 10287 |
| siRNA 4799 | 4799 | 10289 |
| siRNA 4800 | 4800 | 10290 |
| siRNA 4802 | 4802 | 10292 |
| siRNA 4805 | 4805 | 10295 |
| siRNA 4807 | 4807 | 10297 |
| siRNA 4852 | 4852 | 10342 |
| siRNA 4862 | 4862 | 10352 |
| siRNA 4863 | 4863 | 10353 |
| siRNA 4902 | 4902 | 10392 |
| siRNA 4903 | 4903 | 10393 |
| siRNA 4905 | 4905 | 10395 |
| siRNA 4906 | 4906 | 10396 |
| siRNA 4910 | 4910 | 10400 |
| siRNA 4911 | 4911 | 10401 |
| siRNA 4915 | 4915 | 10405 |
| siRNA 4932 | 4932 | 10422 |
| siRNA 4934 | 4934 | 10424 |
| siRNA 4935 | 4935 | 10425 |
| siRNA 4936 | 4936 | 10426 |
| siRNA 4943 | 4943 | 10433 |
| siRNA 4945 | 4945 | 10435 |
| siRNA 4947 | 4947 | 10437 |
| siRNA 4975 | 4975 | 10465 |
| siRNA 4976 | 4976 | 10466 |
| siRNA 4977 | 4977 | 10467 |
| siRNA 4981 | 4981 | 10471 |
| siRNA 4982 | 4982 | 10472 |
| siRNA 4983 | 4983 | 10473 |
| siRNA 4984 | 4984 | 10474 |
| siRNA 4989 | 4989 | 10479 |
| siRNA 4990 | 4990 | 10480 |
| siRNA 4991 | 4991 | 10481 |
| siRNA 4992 | 4992 | 10482 |
| siRNA 4993 | 4993 | 10483 |
| siRNA 4994 | 4994 | 10484 |
| siRNA 4996 | 4996 | 10486 |
| siRNA 4997 | 4997 | 10487 |
| siRNA 4999 | 4999 | 10489 |
| siRNA 5000 | 5000 | 10490 |
| siRNA 5001 | 5001 | 10491 |
| siRNA 5002 | 5002 | 10492 |
| siRNA 5004 | 5004 | 10494 |
| siRNA 5014 | 5014 | 10504 |
| siRNA 5017 | 5017 | 10507 |
| siRNA 5020 | 5020 | 10510 |
| siRNA 5027 | 5027 | 10517 |
| siRNA 5063 | 5063 | 10553 |
| siRNA 5071 | 5071 | 10561 |
| siRNA 5072 | 5072 | 10562 |
| siRNA 5073 | 5073 | 10563 |
| siRNA 5092 | 5092 | 10582 |
| siRNA 5097 | 5097 | 10587 |
| siRNA 5100 | 5100 | 10590 |
| siRNA 5103 | 5103 | 10593 |
| siRNA 5106 | 5106 | 10596 |
| siRNA 5109 | 5109 | 10599 |
| siRNA 5118 | 5118 | 10608 |
| siRNA 5119 | 5119 | 10609 |
| siRNA 5120 | 5120 | 10610 |
| siRNA 5124 | 5124 | 10614 |
| siRNA 5126 | 5126 | 10616 |
| siRNA 5152 | 5152 | 10642 |
| siRNA 5153 | 5153 | 10643 |
| siRNA 5157 | 5157 | 10647 |
| siRNA 5158 | 5158 | 10648 |
| siRNA 5162 | 5162 | 10652 |
| siRNA 5164 | 5164 | 10654 |
| siRNA 5166 | 5166 | 10656 |
| siRNA 5177 | 5177 | 10667 |
| siRNA 5182 | 5182 | 10672 |
| siRNA 5184 | 5184 | 10674 |
| siRNA 5186 | 5186 | 10676 |
| siRNA 5189 | 5189 | 10679 |
| siRNA 5190 | 5190 | 10680 |
| siRNA 5191 | 5191 | 10681 |
| siRNA 5193 | 5193 | 10683 |
| siRNA 5194 | 5194 | 10684 |
| siRNA 5206 | 5206 | 10696 |
| siRNA 5207 | 5207 | 10697 |
| siRNA 5208 | 5208 | 10698 |
| siRNA 5210 | 5210 | 10700 |
| siRNA 5211 | 5211 | 10701 |
| siRNA 5214 | 5214 | 10704 |
| siRNA 5216 | 5216 | 10706 |
| siRNA 5217 | 5217 | 10707 |
| siRNA 5218 | 5218 | 10708 |
| siRNA 5274 | 5274 | 10764 |
| siRNA 5280 | 5280 | 10770 |
| siRNA 5291 | 5291 | 10781 |
| siRNA 5297 | 5297 | 10787 |
| siRNA 5305 | 5305 | 10795 |
| siRNA 5306 | 5306 | 10796 |
| siRNA 5310 | 5310 | 10800 |
| siRNA 5313 | 5313 | 10803 |
| siRNA 5334 | 5334 | 10824 |
| siRNA 5335 | 5335 | 10825 |
| siRNA 5337 | 5337 | 10827 |
| siRNA 5338 | 5338 | 10828 |
| siRNA 5339 | 5339 | 10829 |
| siRNA 5340 | 5340 | 10830 |
| siRNA 5341 | 5341 | 10831 |
| siRNA 5343 | 5343 | 10833 |
| siRNA 5344 | 5344 | 10834 |
| siRNA 5346 | 5346 | 10836 |
| siRNA 5349 | 5349 | 10839 |
| siRNA 5350 | 5350 | 10840 |
| siRNA 5352 | 5352 | 10842 |
| siRNA 5363 | 5363 | 10853 |
| siRNA 5369 | 5369 | 10859 |
| siRNA 5374 | 5374 | 10864 |
| siRNA 5375 | 5375 | 10865 |
| siRNA 5385 | 5385 | 10875 |
| siRNA 5392 | 5392 | 10882 |
| siRNA 5393 | 5393 | 10883 |
| siRNA 5396 | 5396 | 10886 |
| siRNA 5400 | 5400 | 10890 |
| siRNA 5404 | 5404 | 10894 |
| siRNA 5405 | 5405 | 10895 |
| siRNA 5408 | 5408 | 10898 |
| siRNA 5409 | 5409 | 10899 |
| siRNA 5410 | 5410 | 10900 |
| siRNA 5415 | 5415 | 10905 |
| siRNA 5423 | 5423 | 10913 |
| siRNA 5427 | 5427 | 10917 |
| siRNA 5431 | 5431 | 10921 |
| siRNA 5435 | 5435 | 10925 |
| siRNA 5443 | 5443 | 10933 |
| siRNA 5449 | 5449 | 10939 |
| siRNA 5450 | 5450 | 10940 |
| siRNA 5455 | 5455 | 10945 |
| siRNA 5476 | 5476 | 10966 |
| siRNA 5478 | 5478 | 10968 |
| siRNA 5482 | 5482 | 10972 |
| siRNA 5484 | 5484 | 10974 |
| siRNA 5485 | 5485 | 10975 |

The siRNAs in subset B had the following characteristics: Cross-reactivity With 9mer in human SOS2 mRNA; Specificity category: For human: AS2 or better, SS3 or better; miRNA seeds: AS+SS strand: seed region not conserved in human, mouse, and rat and not present in >4 species; Off-target frequency: ≤20 human off-targets matched with 2 mismatches in antisense strand; and SNPs: siRNA target sites do not harbor SNPs with a MAF≤1 (pos. 2-18).

The siRNA sequences in subset B were further selected for absence of seed regions in the AS strand that are identical to a seed region of known human miRNA to yield subset C. Subset C includes 771 siRNAs whose base sequences are shown in Table 17.

TABLE 17
Subset C

| | Sense | Antisense |
|---|---|---|
| siRNA Name | SEQ ID NO: | SEQ ID NO: |
| siRNA 52 | 52 | 5542 |
| siRNA 72 | 72 | 5562 |
| siRNA 105 | 105 | 5595 |
| siRNA 106 | 106 | 5596 |
| siRNA 108 | 108 | 5598 |
| siRNA 158 | 158 | 5648 |
| siRNA 159 | 159 | 5649 |
| siRNA 160 | 160 | 5650 |
| siRNA 164 | 164 | 5654 |
| siRNA 165 | 165 | 5655 |
| siRNA 200 | 200 | 5690 |
| siRNA 224 | 224 | 5714 |
| siRNA 236 | 236 | 5726 |
| siRNA 258 | 258 | 5748 |
| siRNA 266 | 266 | 5756 |
| siRNA 275 | 275 | 5765 |
| siRNA 281 | 281 | 5771 |
| siRNA 282 | 282 | 5772 |
| siRNA 301 | 301 | 5791 |
| siRNA 303 | 303 | 5793 |
| siRNA 305 | 305 | 5795 |
| siRNA 313 | 313 | 5803 |
| siRNA 317 | 317 | 5807 |
| siRNA 331 | 331 | 5821 |
| siRNA 332 | 332 | 5822 |
| siRNA 336 | 336 | 5826 |
| siRNA 338 | 338 | 5828 |
| siRNA 342 | 342 | 5832 |
| siRNA 350 | 350 | 5840 |
| siRNA 353 | 353 | 5843 |
| siRNA 354 | 354 | 5844 |
| siRNA 356 | 356 | 5846 |
| siRNA 368 | 368 | 5858 |
| siRNA 370 | 370 | 5860 |
| siRNA 376 | 376 | 5866 |
| siRNA 381 | 381 | 5871 |
| siRNA 394 | 394 | 5884 |
| siRNA 400 | 400 | 5890 |
| siRNA 403 | 403 | 5893 |
| siRNA 404 | 404 | 5894 |
| siRNA 405 | 405 | 5895 |
| siRNA 415 | 415 | 5905 |
| siRNA 424 | 424 | 5914 |
| siRNA 457 | 457 | 5947 |
| siRNA 463 | 463 | 5953 |
| siRNA 482 | 482 | 5972 |
| siRNA 488 | 488 | 5978 |
| siRNA 494 | 494 | 5984 |
| siRNA 509 | 509 | 5999 |
| siRNA 510 | 510 | 6000 |
| siRNA 514 | 514 | 6004 |
| siRNA 523 | 523 | 6013 |
| siRNA 526 | 526 | 6016 |
| siRNA 528 | 528 | 6018 |
| siRNA 538 | 538 | 6028 |
| siRNA 541 | 541 | 6031 |
| siRNA 546 | 546 | 6036 |
| siRNA 552 | 552 | 6042 |
| siRNA 556 | 556 | 6046 |
| siRNA 562 | 562 | 6052 |
| siRNA 570 | 570 | 6060 |
| siRNA 573 | 573 | 6063 |
| siRNA 574 | 574 | 6064 |
| siRNA 575 | 575 | 6065 |
| siRNA 587 | 587 | 6077 |
| siRNA 589 | 589 | 6079 |
| siRNA 602 | 602 | 6092 |
| siRNA 617 | 617 | 6107 |
| siRNA 619 | 619 | 6109 |
| siRNA 620 | 620 | 6110 |
| siRNA 622 | 622 | 6112 |
| siRNA 627 | 627 | 6117 |
| siRNA 633 | 633 | 6123 |
| siRNA 638 | 638 | 6128 |
| siRNA 639 | 639 | 6129 |
| siRNA 640 | 640 | 6130 |
| siRNA 643 | 643 | 6133 |
| siRNA 644 | 644 | 6134 |
| siRNA 647 | 647 | 6137 |
| siRNA 650 | 650 | 6140 |
| siRNA 651 | 651 | 6141 |
| siRNA 652 | 652 | 6142 |
| siRNA 657 | 657 | 6147 |
| siRNA 660 | 660 | 6150 |
| siRNA 664 | 664 | 6154 |
| siRNA 665 | 665 | 6155 |
| siRNA 671 | 671 | 6161 |
| siRNA 678 | 678 | 6168 |
| siRNA 679 | 679 | 6169 |
| siRNA 680 | 680 | 6170 |
| siRNA 682 | 682 | 6172 |
| siRNA 685 | 685 | 6175 |
| siRNA 689 | 689 | 6179 |
| siRNA 690 | 690 | 6180 |
| siRNA 691 | 691 | 6181 |
| siRNA 692 | 692 | 6182 |
| siRNA 719 | 719 | 6209 |
| siRNA 736 | 736 | 6226 |
| siRNA 737 | 737 | 6227 |
| siRNA 738 | 738 | 6228 |
| siRNA 739 | 739 | 6229 |
| siRNA 740 | 740 | 6230 |
| siRNA 743 | 743 | 6233 |
| siRNA 744 | 744 | 6234 |
| siRNA 749 | 749 | 6239 |
| siRNA 750 | 750 | 6240 |
| siRNA 766 | 766 | 6256 |
| siRNA 771 | 771 | 6261 |
| siRNA 782 | 782 | 6272 |
| siRNA 783 | 783 | 6273 |
| siRNA 784 | 784 | 6274 |
| siRNA 785 | 785 | 6275 |
| siRNA 786 | 786 | 6276 |
| siRNA 787 | 787 | 6277 |
| siRNA 788 | 788 | 5278 |
| siRNA 789 | 789 | 6279 |
| siRNA 792 | 792 | 6282 |
| siRNA 795 | 795 | 6285 |
| siRNA 796 | 796 | 6286 |
| siRNA 797 | 797 | 6287 |
| siRNA 810 | 810 | 6300 |
| siRNA 813 | 813 | 6303 |
| siRNA 818 | 818 | 6308 |
| siRNA 819 | 819 | 6309 |
| siRNA 853 | 853 | 6343 |
| siRNA 854 | 854 | 6344 |
| siRNA 856 | 856 | 6346 |
| siRNA 857 | 857 | 6347 |
| siRNA 872 | 872 | 6362 |
| siRNA 877 | 877 | 6367 |
| siRNA 887 | 887 | 6377 |
| siRNA 888 | 888 | 6378 |
| siRNA 922 | 922 | 6412 |
| siRNA 924 | 924 | 6414 |
| siRNA 925 | 925 | 6415 |
| siRNA 926 | 926 | 6416 |
| siRNA 928 | 928 | 6418 |
| siRNA 930 | 930 | 6420 |
| siRNA 933 | 933 | 6423 |
| siRNA 934 | 934 | 6424 |
| siRNA 938 | 938 | 6428 |
| siRNA 941 | 941 | 6431 |
| siRNA 942 | 942 | 6432 |
| siRNA 953 | 953 | 6443 |
| siRNA 954 | 954 | 6444 |
| siRNA 961 | 961 | 6451 |
| siRNA 967 | 967 | 6457 |
| siRNA 973 | 973 | 6463 |
| siRNA 981 | 981 | 6471 |
| siRNA 999 | 999 | 6489 |
| siRNA 1000 | 1000 | 6490 |
| siRNA 1001 | 1001 | 6491 |
| siRNA 1006 | 1006 | 6496 |
| siRNA 1007 | 1007 | 6497 |
| siRNA 1040 | 1040 | 6530 |
| siRNA 1052 | 1052 | 6542 |
| siRNA 1053 | 1053 | 6543 |
| siRNA 1056 | 1056 | 6546 |
| siRNA 1075 | 1075 | 6565 |
| siRNA 1082 | 1082 | 6572 |
| siRNA 1084 | 1084 | 6574 |
| siRNA 1086 | 1086 | 6576 |
| siRNA 1087 | 1087 | 6577 |
| siRNA 1153 | 1153 | 6643 |
| siRNA 1156 | 1156 | 6646 |
| siRNA 1157 | 1157 | 6647 |
| siRNA 1158 | 1158 | 6648 |
| siRNA 1159 | 1159 | 6649 |
| siRNA 1175 | 1175 | 6665 |
| siRNA 1185 | 1185 | 6675 |
| siRNA 1196 | 1196 | 6686 |
| siRNA 1224 | 1224 | 6714 |
| siRNA 1255 | 1255 | 6745 |
| siRNA 1279 | 1279 | 6769 |
| siRNA 1280 | 1280 | 6770 |
| siRNA 1282 | 1282 | 6772 |
| siRNA 1286 | 1286 | 6776 |
| siRNA 1294 | 1294 | 6784 |
| siRNA 1296 | 1296 | 6786 |
| siRNA 1301 | 1301 | 6791 |
| siRNA 1302 | 1302 | 6792 |
| siRNA 1303 | 1303 | 6793 |
| siRNA 1318 | 1318 | 6808 |
| siRNA 1322 | 1322 | 6812 |
| siRNA 1343 | 1343 | 6833 |
| siRNA 1344 | 1344 | 6834 |
| siRNA 1346 | 1346 | 6836 |
| siRNA 1347 | 1347 | 6837 |
| siRNA 1363 | 1363 | 6853 |
| siRNA 1395 | 1395 | 6885 |
| siRNA 1398 | 1398 | 6888 |
| siRNA 1405 | 1405 | 6895 |
| siRNA 1406 | 1406 | 6896 |
| siRNA 1408 | 1408 | 6898 |
| siRNA 1430 | 1430 | 6920 |
| siRNA 1441 | 1441 | 6931 |
| siRNA 1442 | 1442 | 6932 |
| siRNA 1445 | 1445 | 6935 |
| siRNA 1449 | 1449 | 6939 |
| siRNA 1452 | 1452 | 6942 |
| siRNA 1453 | 1453 | 6943 |
| siRNA 1459 | 1459 | 6949 |
| siRNA 1460 | 1460 | 6950 |
| siRNA 1462 | 1462 | 6952 |
| siRNA 1463 | 1463 | 6953 |
| siRNA 1465 | 1465 | 6955 |
| siRNA 1466 | 1466 | 6956 |
| siRNA 1467 | 1467 | 6957 |
| siRNA 1468 | 1468 | 6958 |
| siRNA 1469 | 1469 | 6959 |
| siRNA 1470 | 1470 | 6960 |
| siRNA 1474 | 1474 | 6964 |
| siRNA 1475 | 1475 | 6965 |
| siRNA 1495 | 1495 | 6985 |
| siRNA 1498 | 1498 | 6988 |
| siRNA 1503 | 1503 | 6993 |
| siRNA 1505 | 1505 | 6995 |
| siRNA 1510 | 1510 | 7000 |
| siRNA 1529 | 1529 | 7019 |
| siRNA 1533 | 1533 | 7023 |
| siRNA 1535 | 1535 | 7025 |
| siRNA 1560 | 1560 | 7050 |
| siRNA 1568 | 1568 | 7058 |
| siRNA 1569 | 1569 | 7059 |
| siRNA 1570 | 1570 | 7060 |
| siRNA 1572 | 1572 | 7062 |
| siRNA 1575 | 1575 | 7065 |
| siRNA 1587 | 1587 | 7077 |
| siRNA 1589 | 1589 | 7079 |
| siRNA 1596 | 1596 | 7086 |
| siRNA 1614 | 1614 | 7104 |
| siRNA 1621 | 1621 | 7111 |
| siRNA 1622 | 1622 | 7112 |
| siRNA 1623 | 1623 | 7113 |
| siRNA 1628 | 1628 | 7118 |
| siRNA 1631 | 1631 | 7121 |
| siRNA 1632 | 1632 | 7122 |
| siRNA 1633 | 1633 | 7123 |
| siRNA 1634 | 1634 | 7124 |
| siRNA 1635 | 1635 | 7125 |
| siRNA 1636 | 1636 | 7126 |
| siRNA 1638 | 1638 | 7128 |
| siRNA 1644 | 1644 | 7134 |
| siRNA 1648 | 1648 | 7138 |
| siRNA 1649 | 1649 | 7139 |
| siRNA 1650 | 1650 | 7140 |
| siRNA 1651 | 1651 | 7141 |
| siRNA 1652 | 1652 | 7142 |
| siRNA 1654 | 1654 | 7144 |
| siRNA 1656 | 1656 | 7146 |
| siRNA 1659 | 1659 | 7149 |
| siRNA 1683 | 1683 | 7173 |
| siRNA 1684 | 1684 | 7174 |
| siRNA 1689 | 1689 | 7179 |
| siRNA 1697 | 1697 | 7187 |
| siRNA 1699 | 1699 | 7189 |
| siRNA 1706 | 1706 | 7196 |
| siRNA 1708 | 1708 | 7198 |
| siRNA 1712 | 1712 | 7202 |
| siRNA 1720 | 1720 | 7210 |
| siRNA 1802 | 1802 | 7292 |
| siRNA 1806 | 1806 | 7296 |
| siRNA 1814 | 1814 | 7304 |
| siRNA 1825 | 1825 | 7315 |
| siRNA 1826 | 1826 | 7316 |
| siRNA 1832 | 1832 | 7322 |
| siRNA 1844 | 1844 | 7334 |
| siRNA 1846 | 1846 | 7336 |
| siRNA 1851 | 1851 | 7341 |
| siRNA 1861 | 1861 | 7351 |
| siRNA 1865 | 1865 | 7355 |
| siRNA 1897 | 1897 | 7387 |
| siRNA 1909 | 1909 | 7399 |
| siRNA 1912 | 1912 | 7402 |
| siRNA 1914 | 1914 | 7404 |
| siRNA 1915 | 1915 | 7405 |
| siRNA 1916 | 1916 | 7406 |
| siRNA 1917 | 1917 | 7407 |
| siRNA 1918 | 1918 | 7408 |
| siRNA 1921 | 1921 | 7411 |
| siRNA 1922 | 1922 | 7412 |
| siRNA 1926 | 1926 | 7416 |
| siRNA 1927 | 1927 | 7417 |
| siRNA 1928 | 1928 | 7418 |
| siRNA 1929 | 1929 | 7419 |
| siRNA 1935 | 1935 | 7425 |
| siRNA 1936 | 1936 | 7426 |
| siRNA 1937 | 1937 | 7427 |
| siRNA 1938 | 1938 | 7428 |
| siRNA 1940 | 1940 | 7430 |
| siRNA 1948 | 1948 | 7438 |
| siRNA 1951 | 1951 | 7441 |
| siRNA 1980 | 1980 | 7470 |
| siRNA 1981 | 1981 | 7471 |
| siRNA 1984 | 1984 | 7474 |
| siRNA 1986 | 1986 | 7476 |
| siRNA 1987 | 1987 | 7477 |
| siRNA 1991 | 1991 | 7481 |
| siRNA 1992 | 1992 | 7482 |
| siRNA 1995 | 1995 | 7485 |
| siRNA 1996 | 1996 | 7486 |
| siRNA 1999 | 1999 | 7489 |
| siRNA 2000 | 2000 | 7490 |
| siRNA 2002 | 2002 | 7492 |
| siRNA 2005 | 2005 | 7495 |
| siRNA 2014 | 2014 | 7504 |
| siRNA 2020 | 2020 | 7510 |
| siRNA 2049 | 2049 | 7539 |
| siRNA 2051 | 2051 | 7541 |
| siRNA 2057 | 2057 | 7547 |
| siRNA 2062 | 2062 | 7552 |
| siRNA 2065 | 2065 | 7555 |
| siRNA 2073 | 2073 | 7563 |
| siRNA 2074 | 2074 | 7564 |
| siRNA 2077 | 2077 | 7567 |
| siRNA 2096 | 2096 | 7586 |
| siRNA 2112 | 2112 | 7602 |
| siRNA 2116 | 2116 | 7606 |
| siRNA 2120 | 2120 | 7610 |
| siRNA 2147 | 2147 | 7637 |
| siRNA 2148 | 2148 | 7638 |
| siRNA 2149 | 2149 | 7639 |
| siRNA 2150 | 2150 | 7640 |
| siRNA 2151 | 2151 | 7641 |
| siRNA 2154 | 2154 | 7644 |
| siRNA 2157 | 2157 | 7647 |
| siRNA 2158 | 2158 | 7648 |
| siRNA 2164 | 2164 | 7654 |
| siRNA 2167 | 2167 | 7657 |
| siRNA 2168 | 2168 | 7658 |
| siRNA 2170 | 2170 | 7660 |
| siRNA 2172 | 2172 | 7662 |
| siRNA 2193 | 2193 | 7683 |
| siRNA 2202 | 2202 | 7692 |
| siRNA 2209 | 2209 | 7699 |
| siRNA 2213 | 2213 | 7703 |
| siRNA 2214 | 2214 | 7704 |
| siRNA 2244 | 2244 | 7734 |
| siRNA 2245 | 2245 | 7735 |
| siRNA 2246 | 2246 | 7736 |
| siRNA 2251 | 2251 | 7741 |
| siRNA 2252 | 2252 | 7742 |
| siRNA 2254 | 2254 | 7744 |
| siRNA 2257 | 2257 | 7747 |
| siRNA 2286 | 2286 | 7776 |
| siRNA 2287 | 2287 | 7777 |
| siRNA 2306 | 2306 | 7796 |
| siRNA 2307 | 2307 | 7797 |
| siRNA 2312 | 2312 | 7802 |
| siRNA 2313 | 2313 | 7803 |
| siRNA 2314 | 2314 | 7804 |
| siRNA 2318 | 2318 | 7808 |
| siRNA 2322 | 2322 | 7812 |
| siRNA 2324 | 2324 | 7814 |
| siRNA 2328 | 2328 | 7818 |
| siRNA 2329 | 2329 | 7819 |
| siRNA 2332 | 2332 | 7822 |
| siRNA 2336 | 2336 | 7826 |
| siRNA 2338 | 2338 | 7828 |
| siRNA 2339 | 2339 | 7829 |
| siRNA 2340 | 2340 | 7830 |
| siRNA 2355 | 2355 | 7845 |
| siRNA 2356 | 2356 | 7846 |
| siRNA 2357 | 2357 | 7847 |
| siRNA 2360 | 2360 | 7850 |
| siRNA 2362 | 2362 | 7852 |
| siRNA 2364 | 2364 | 7854 |
| siRNA 2365 | 2365 | 7855 |
| siRNA 2366 | 2366 | 7856 |
| siRNA 2368 | 2368 | 7858 |
| siRNA 2369 | 2369 | 7859 |
| siRNA 2370 | 2370 | 7860 |
| siRNA 2403 | 2403 | 7893 |
| siRNA 2417 | 2417 | 7907 |
| siRNA 2418 | 2418 | 7908 |
| siRNA 2436 | 2436 | 7926 |
| siRNA 2470 | 2470 | 7960 |
| siRNA 2471 | 2471 | 7961 |
| siRNA 2472 | 2472 | 7962 |
| siRNA 2477 | 2477 | 7967 |
| siRNA 2480 | 2480 | 7970 |
| siRNA 2486 | 2486 | 7976 |
| siRNA 2528 | 2528 | 8018 |
| siRNA 2530 | 2530 | 8020 |
| siRNA 2531 | 2531 | 8021 |
| siRNA 2532 | 2532 | 8022 |
| siRNA 2568 | 2568 | 8058 |
| siRNA 2598 | 2598 | 8088 |
| siRNA 2619 | 2619 | 8109 |
| siRNA 2620 | 2620 | 8110 |
| siRNA 2623 | 2623 | 8113 |
| siRNA 2630 | 2630 | 8120 |
| siRNA 2631 | 2631 | 8121 |
| siRNA 2632 | 2632 | 8122 |
| siRNA 2633 | 2633 | 8123 |
| siRNA 2637 | 2637 | 8127 |
| siRNA 2645 | 2645 | 8135 |
| siRNA 2646 | 2646 | 8136 |
| siRNA 2673 | 2673 | 8163 |
| siRNA 2675 | 2675 | 8165 |
| siRNA 2679 | 2679 | 8169 |
| siRNA 2680 | 2680 | 8170 |
| siRNA 2682 | 2682 | 8172 |
| siRNA 2699 | 2699 | 8189 |
| siRNA 2708 | 2708 | 8198 |
| siRNA 2713 | 2713 | 8203 |
| siRNA 2755 | 2755 | 8245 |
| siRNA 2756 | 2756 | 8246 |
| siRNA 2757 | 2757 | 8247 |
| siRNA 2759 | 2759 | 8249 |
| siRNA 2760 | 2760 | 8250 |
| siRNA 2762 | 2762 | 8252 |
| siRNA 2763 | 2763 | 8253 |
| siRNA 2764 | 2764 | 8254 |
| siRNA 2767 | 2767 | 8257 |
| siRNA 2815 | 2815 | 8305 |
| siRNA 2816 | 2816 | 8306 |
| siRNA 2817 | 2817 | 8307 |
| siRNA 2821 | 2821 | 8311 |
| siRNA 2825 | 2825 | 8315 |
| siRNA 2828 | 2828 | 8318 |
| siRNA 2829 | 2829 | 8319 |
| siRNA 2830 | 2830 | 8320 |
| siRNA 2834 | 2834 | 8324 |
| siRNA 2836 | 2836 | 8326 |
| siRNA 2841 | 2841 | 8331 |
| siRNA 2842 | 2842 | 8332 |
| siRNA 2847 | 2847 | 8337 |
| siRNA 2866 | 2866 | 8356 |
| siRNA 2882 | 2882 | 8372 |
| siRNA 2883 | 2883 | 8373 |
| siRNA 2886 | 2886 | 8376 |
| siRNA 2888 | 2888 | 8378 |
| siRNA 2890 | 2890 | 8380 |
| siRNA 2898 | 2898 | 8388 |
| siRNA 2902 | 2902 | 8392 |
| siRNA 2908 | 2908 | 8398 |
| siRNA 2928 | 2928 | 8418 |
| siRNA 2929 | 2929 | 8419 |
| siRNA 2932 | 2932 | 8422 |
| siRNA 2938 | 2938 | 8428 |
| siRNA 2939 | 2939 | 8429 |
| siRNA 2944 | 2944 | 8434 |
| siRNA 2946 | 2946 | 8436 |
| siRNA 2947 | 2947 | 8437 |
| siRNA 2981 | 2981 | 8471 |
| siRNA 2982 | 2982 | 8472 |
| siRNA 2983 | 2983 | 8473 |
| siRNA 2984 | 2984 | 8474 |
| siRNA 2985 | 2985 | 8475 |
| siRNA 2986 | 2986 | 8476 |
| siRNA 2987 | 2987 | 8477 |
| siRNA 2998 | 2998 | 8488 |
| siRNA 3024 | 3024 | 8514 |
| siRNA 3025 | 3025 | 8515 |
| siRNA 3035 | 3035 | 8525 |
| siRNA 3038 | 3038 | 8528 |
| siRNA 3042 | 3042 | 8532 |
| siRNA 3053 | 3053 | 8543 |
| siRNA 3093 | 3093 | 8583 |
| siRNA 3094 | 3094 | 8584 |
| siRNA 3140 | 3140 | 8630 |
| siRNA 3146 | 3146 | 8636 |
| siRNA 3164 | 3164 | 8654 |
| siRNA 3209 | 3209 | 8699 |
| siRNA 3218 | 3218 | 8708 |
| siRNA 3221 | 3221 | 8711 |
| siRNA 3222 | 3222 | 8712 |
| siRNA 3223 | 3223 | 8713 |
| siRNA 3231 | 3231 | 8721 |
| siRNA 3232 | 3232 | 8722 |
| siRNA 3238 | 3238 | 8728 |
| siRNA 3247 | 3247 | 8737 |
| siRNA 3259 | 3259 | 8749 |
| siRNA 3263 | 3263 | 8753 |
| siRNA 3305 | 3305 | 8795 |
| siRNA 3310 | 3310 | 8800 |
| siRNA 3330 | 3330 | 8820 |
| siRNA 3333 | 3333 | 8823 |
| siRNA 3335 | 3335 | 8825 |
| siRNA 3339 | 3339 | 8829 |
| siRNA 3342 | 3342 | 8832 |
| siRNA 3349 | 3349 | 8839 |
| siRNA 3350 | 3350 | 8840 |
| siRNA 3351 | 3351 | 8841 |
| siRNA 3352 | 3352 | 8842 |
| siRNA 3353 | 3353 | 8843 |
| siRNA 3354 | 3354 | 8844 |
| siRNA 3357 | 3357 | 8847 |
| siRNA 3358 | 3358 | 8848 |
| siRNA 3359 | 3359 | 8849 |
| siRNA 3364 | 3364 | 8854 |
| siRNA 3396 | 3396 | 8886 |
| siRNA 3397 | 3397 | 8887 |
| siRNA 3405 | 3405 | 8895 |
| siRNA 3411 | 3411 | 8901 |
| siRNA 3412 | 3412 | 8902 |
| siRNA 3413 | 3413 | 8903 |
| siRNA 3416 | 3416 | 8906 |
| siRNA 3417 | 3417 | 8907 |
| siRNA 3418 | 3418 | 8908 |
| siRNA 3425 | 3425 | 8915 |
| siRNA 3432 | 3432 | 8922 |
| siRNA 3435 | 3435 | 8925 |
| siRNA 3436 | 3436 | 8926 |
| siRNA 3437 | 3437 | 8927 |
| siRNA 3438 | 3438 | 8928 |
| siRNA 3439 | 3439 | 8929 |
| siRNA 3444 | 3444 | 8934 |
| siRNA 3449 | 3449 | 8939 |
| siRNA 3450 | 3450 | 8940 |
| siRNA 3456 | 3456 | 8946 |
| siRNA 3457 | 3457 | 8947 |
| siRNA 3460 | 3460 | 8950 |
| siRNA 3461 | 3461 | 8951 |
| siRNA 3472 | 3472 | 8962 |
| siRNA 3487 | 3487 | 8977 |
| siRNA 3488 | 3488 | 8978 |
| siRNA 3489 | 3489 | 8979 |
| siRNA 3490 | 3490 | 8980 |
| siRNA 3491 | 3491 | 8981 |
| siRNA 3492 | 3492 | 8982 |
| siRNA 3500 | 3500 | 8990 |
| siRNA 3502 | 3502 | 8992 |
| siRNA 3512 | 3512 | 9002 |
| siRNA 3540 | 3540 | 9030 |
| siRNA 3546 | 3546 | 9036 |
| siRNA 3549 | 3549 | 9039 |
| siRNA 3560 | 3560 | 9050 |
| siRNA 3561 | 3561 | 9051 |
| siRNA 3564 | 3564 | 9054 |
| siRNA 3566 | 3566 | 9056 |
| siRNA 3582 | 3582 | 9072 |
| siRNA 3585 | 3585 | 9075 |
| siRNA 3586 | 3586 | 9076 |
| siRNA 3587 | 3587 | 9077 |
| siRNA 3602 | 3602 | 9092 |
| siRNA 3604 | 3604 | 9094 |
| siRNA 3605 | 3605 | 9095 |
| siRNA 3629 | 3629 | 9119 |
| siRNA 3634 | 3634 | 9124 |
| siRNA 3639 | 3639 | 9129 |
| siRNA 3641 | 3641 | 9131 |
| siRNA 3677 | 3677 | 9167 |
| siRNA 3686 | 3686 | 9176 |
| siRNA 3688 | 3688 | 9178 |
| siRNA 3691 | 3691 | 9181 |
| siRNA 3692 | 3692 | 9182 |
| siRNA 3695 | 3695 | 9185 |
| siRNA 3703 | 3703 | 9193 |
| siRNA 3712 | 3712 | 9202 |
| siRNA 3746 | 3746 | 9236 |
| siRNA 3747 | 3747 | 9237 |
| siRNA 3748 | 3748 | 9238 |
| siRNA 3756 | 3756 | 9246 |
| siRNA 3792 | 3792 | 9282 |
| siRNA 3802 | 3802 | 9292 |
| siRNA 3803 | 3803 | 9293 |
| siRNA 3810 | 3810 | 9300 |
| siRNA 3838 | 3838 | 9328 |
| siRNA 3869 | 3869 | 9359 |
| siRNA 3883 | 3883 | 9373 |
| siRNA 3897 | 3897 | 9387 |
| siRNA 3922 | 3922 | 9412 |
| siRNA 3934 | 3934 | 9424 |
| siRNA 3936 | 3936 | 9426 |
| siRNA 3943 | 3943 | 9433 |
| siRNA 3945 | 3945 | 9435 |
| siRNA 3960 | 3960 | 9450 |
| siRNA 3970 | 3970 | 9460 |
| siRNA 3988 | 3988 | 9478 |
| siRNA 4010 | 4010 | 9500 |
| siRNA 4025 | 4025 | 9515 |
| siRNA 4036 | 4036 | 9526 |
| siRNA 4038 | 4038 | 9528 |
| siRNA 4040 | 4040 | 9530 |
| siRNA 4041 | 4041 | 9531 |
| siRNA 4043 | 4043 | 9533 |
| siRNA 4050 | 4050 | 9540 |
| siRNA 4051 | 4051 | 9541 |
| siRNA 4052 | 4052 | 9542 |
| siRNA 4053 | 4053 | 9543 |
| siRNA 4054 | 4054 | 9544 |
| siRNA 4056 | 4056 | 9546 |
| siRNA 4058 | 4058 | 9548 |
| siRNA 4070 | 4070 | 9560 |
| siRNA 4077 | 4077 | 9567 |
| siRNA 4112 | 4112 | 9602 |
| siRNA 4125 | 4125 | 9615 |
| siRNA 4190 | 4190 | 9680 |
| siRNA 4192 | 4192 | 9682 |
| siRNA 4199 | 4199 | 9689 |
| siRNA 4207 | 4207 | 9697 |
| siRNA 4209 | 4209 | 9699 |
| siRNA 4210 | 4210 | 9700 |
| siRNA 4211 | 4211 | 9701 |
| siRNA 4214 | 4214 | 9704 |
| siRNA 4216 | 4216 | 9706 |
| siRNA 4219 | 4219 | 9709 |
| siRNA 4222 | 4222 | 9712 |
| siRNA 4223 | 4223 | 9713 |
| siRNA 4224 | 4224 | 9714 |
| siRNA 4225 | 4225 | 9715 |
| siRNA 4226 | 4226 | 9716 |
| siRNA 4228 | 4228 | 9718 |
| siRNA 4235 | 4235 | 9725 |
| siRNA 4237 | 4237 | 9727 |
| siRNA 4238 | 4238 | 9728 |
| siRNA 4242 | 4242 | 9732 |
| siRNA 4243 | 4243 | 9733 |
| siRNA 4245 | 4245 | 9735 |
| siRNA 4254 | 4254 | 9744 |
| siRNA 4275 | 4275 | 9765 |
| siRNA 4276 | 4276 | 9766 |
| siRNA 4279 | 4279 | 9769 |
| siRNA 4282 | 4282 | 9772 |
| siRNA 4284 | 4284 | 9774 |
| siRNA 4290 | 4290 | 9780 |
| siRNA 4292 | 4292 | 9782 |
| siRNA 4293 | 4293 | 9783 |
| siRNA 4294 | 4294 | 9784 |
| siRNA 4295 | 4295 | 9785 |
| siRNA 4296 | 4296 | 9786 |
| siRNA 4297 | 4297 | 9787 |
| siRNA 4309 | 4309 | 9799 |
| siRNA 4313 | 4313 | 9803 |
| siRNA 4322 | 4322 | 9812 |
| siRNA 4356 | 4356 | 9846 |
| siRNA 4368 | 4368 | 9858 |
| siRNA 4382 | 4382 | 9872 |
| siRNA 4388 | 4388 | 9878 |
| siRNA 4409 | 4409 | 9899 |
| siRNA 4453 | 4453 | 9943 |
| siRNA 4455 | 4455 | 9945 |
| siRNA 4456 | 4456 | 9946 |
| siRNA 4459 | 4459 | 9949 |
| siRNA 4460 | 4460 | 9950 |
| siRNA 4468 | 4468 | 9958 |
| siRNA 4474 | 4474 | 9964 |
| siRNA 4475 | 4475 | 9965 |
| siRNA 4481 | 4481 | 9971 |
| siRNA 4482 | 4482 | 9972 |
| siRNA 4487 | 4487 | 9977 |
| siRNA 4488 | 4488 | 9978 |
| siRNA 4490 | 4490 | 9980 |
| siRNA 4491 | 4491 | 9981 |
| siRNA 4492 | 4492 | 9982 |
| siRNA 4518 | 4518 | 10008 |
| siRNA 4519 | 4519 | 10009 |
| siRNA 4521 | 4521 | 10011 |
| siRNA 4545 | 4545 | 10035 |
| siRNA 4547 | 4547 | 10037 |
| siRNA 4561 | 4561 | 10051 |
| siRNA 4565 | 4565 | 10055 |
| siRNA 4593 | 4593 | 10083 |
| siRNA 4594 | 4594 | 10084 |
| siRNA 4595 | 4595 | 10085 |
| siRNA 4596 | 4596 | 10086 |
| siRNA 4597 | 4597 | 10087 |
| siRNA 4598 | 4598 | 10088 |
| siRNA 4628 | 4628 | 10118 |
| siRNA 4629 | 4629 | 10119 |
| siRNA 4644 | 4644 | 10134 |
| siRNA 4675 | 4675 | 10165 |
| siRNA 4679 | 4679 | 10169 |
| siRNA 4680 | 4680 | 10170 |
| siRNA 4682 | 4682 | 10172 |
| siRNA 4705 | 4705 | 10195 |
| siRNA 4726 | 4726 | 10216 |
| siRNA 4739 | 4739 | 10229 |
| siRNA 4756 | 4756 | 10246 |
| siRNA 4765 | 4765 | 10255 |
| siRNA 4767 | 4767 | 10257 |
| siRNA 4770 | 4770 | 10260 |
| siRNA 4797 | 4797 | 10287 |
| siRNA 4800 | 4800 | 10290 |
| siRNA 4802 | 4802 | 10292 |
| siRNA 4805 | 4805 | 10295 |
| siRNA 4807 | 4807 | 10297 |
| siRNA 4852 | 4852 | 10342 |
| siRNA 4862 | 4862 | 10352 |
| siRNA 4863 | 4863 | 10353 |
| siRNA 4902 | 4902 | 10392 |
| siRNA 4903 | 4903 | 10393 |
| siRNA 4905 | 4905 | 10395 |
| siRNA 4906 | 4906 | 10396 |
| siRNA 4910 | 4910 | 10400 |
| siRNA 4932 | 4932 | 10422 |
| siRNA 4934 | 4934 | 10424 |
| siRNA 4936 | 4936 | 10426 |
| siRNA 4943 | 4943 | 10433 |
| siRNA 4975 | 4975 | 10465 |
| siRNA 4977 | 4977 | 10467 |
| siRNA 4981 | 4981 | 10471 |
| siRNA 4982 | 4982 | 10472 |
| siRNA 4983 | 4983 | 10473 |
| siRNA 4984 | 4984 | 10474 |
| siRNA 4989 | 4989 | 10479 |
| siRNA 4990 | 4990 | 10480 |
| siRNA 4993 | 4993 | 10483 |
| siRNA 4996 | 4996 | 10486 |
| siRNA 4997 | 4997 | 10487 |
| siRNA 4999 | 4999 | 10489 |
| siRNA 5000 | 5000 | 10490 |
| siRNA 5001 | 5001 | 10491 |
| siRNA 5004 | 5004 | 10494 |
| siRNA 5017 | 5017 | 10507 |
| siRNA 5020 | 5020 | 10510 |
| siRNA 5027 | 5027 | 10517 |
| siRNA 5071 | 5071 | 10561 |
| siRNA 5072 | 5072 | 10562 |
| siRNA 5073 | 5073 | 10563 |
| siRNA 5092 | 5092 | 10582 |
| siRNA 5100 | 5100 | 10590 |
| siRNA 5103 | 5103 | 10593 |
| siRNA 5106 | 5106 | 10596 |
| siRNA 5118 | 5118 | 10608 |
| siRNA 5119 | 5119 | 10609 |
| siRNA 5120 | 5120 | 10610 |
| siRNA 5124 | 5124 | 10614 |
| siRNA 5126 | 5126 | 10616 |
| siRNA 5153 | 5153 | 10643 |
| siRNA 5158 | 5158 | 10648 |
| siRNA 5164 | 5164 | 10654 |
| siRNA 5182 | 5182 | 10672 |
| siRNA 5184 | 5184 | 10674 |
| siRNA 5186 | 5186 | 10676 |
| siRNA 5190 | 5190 | 10680 |
| siRNA 5191 | 5191 | 10681 |
| siRNA 5194 | 5194 | 10684 |
| siRNA 5206 | 5206 | 10696 |
| siRNA 5207 | 5207 | 10697 |
| siRNA 5208 | 5208 | 10698 |
| siRNA 5210 | 5210 | 10700 |
| siRNA 5211 | 5211 | 10701 |
| siRNA 5214 | 5214 | 10704 |
| siRNA 5217 | 5217 | 10707 |
| siRNA 5280 | 5280 | 10770 |
| siRNA 5291 | 5291 | 10781 |
| siRNA 5297 | 5297 | 10787 |
| siRNA 5305 | 5305 | 10795 |
| siRNA 5306 | 5306 | 10796 |
| siRNA 5313 | 5313 | 10803 |
| siRNA 5334 | 5334 | 10824 |
| siRNA 5335 | 5335 | 10825 |
| siRNA 5337 | 5337 | 10827 |
| siRNA 5339 | 5339 | 10829 |
| siRNA 5340 | 5340 | 10830 |
| siRNA 5341 | 5341 | 10831 |
| siRNA 5343 | 5343 | 10833 |
| siRNA 5344 | 5344 | 10834 |
| siRNA 5346 | 5346 | 10836 |
| siRNA 5349 | 5349 | 10839 |
| siRNA 5352 | 5352 | 10842 |
| siRNA 5363 | 5363 | 10853 |
| siRNA 5374 | 5374 | 10864 |
| siRNA 5375 | 5375 | 10865 |
| siRNA 5385 | 5385 | 10875 |
| siRNA 5392 | 5392 | 10882 |
| siRNA 5393 | 5393 | 10883 |
| siRNA 5404 | 5404 | 10894 |
| siRNA 5408 | 5408 | 10898 |
| siRNA 5409 | 5409 | 10899 |
| siRNA 5423 | 5423 | 10913 |
| siRNA 5431 | 5431 | 10921 |
| siRNA 5435 | 5435 | 10925 |
| siRNA 5443 | 5443 | 10933 |
| siRNA 5455 | 5455 | 10945 |
| siRNA 5478 | 5478 | 10968 |
| siRNA 5484 | 5484 | 10974 |
| siRNA 5485 | 5485 | 10975 |

The siRNAs in subset C had the following characteristics: Cross-reactivity: With 19mer in human SOS2 mRNA; Specificity category: For human: AS2 or better, SS3 or better; miRNA seeds: AS+SS strand: seed region not conserved in human, mouse, and rat and not present in ≥4 species. AS strand: seed region not identical to seed region of known human miRNA; Off-target frequency: 730 human off-targets matched with 2 mismatches by antisense strand; and SNPs: siRNA target sites do not harbor SNPs with a MAF≥1% (pos. 2-18).

The siRNA sequences in subset C were also selected for absence of seed regions in the AS or S strands that are identical to a seed region of known human miRNA in addition to having an off-target frequency of ≤30 human off-targets matched with 2 mismatches by antisense strand to yield subset D. Subset D includes 489 siRNAs whose base sequences are shown in Table 18.

TABLE 18
Subset D

| | Sense | Antisense |
|---|---|---|
| siRNA Name | SEQ ID NO: | SEQ ID NO: |
| siRNA 72 | 72 | 5562 |
| siRNA 106 | 106 | 5596 |
| siRNA 164 | 164 | 5654 |
| siRNA 224 | 224 | 5714 |
| siRNA 236 | 236 | 5726 |
| siRNA 266 | 266 | 5756 |
| siRNA 281 | 281 | 5771 |
| siRNA 282 | 282 | 5772 |
| siRNA 305 | 305 | 5795 |
| siRNA 313 | 313 | 5803 |
| siRNA 317 | 317 | 5807 |
| siRNA 336 | 336 | 5826 |
| siRNA 338 | 338 | 5828 |
| siRNA 342 | 342 | 5832 |
| siRNA 356 | 356 | 5846 |
| siRNA 381 | 381 | 5871 |
| siRNA 394 | 394 | 5884 |
| siRNA 403 | 403 | 5893 |
| siRNA 415 | 415 | 5905 |
| siRNA 457 | 457 | 5947 |
| siRNA 463 | 463 | 5953 |
| siRNA 482 | 482 | 5972 |
| siRNA 509 | 509 | 5999 |
| siRNA 510 | 510 | 6000 |
| siRNA 514 | 514 | 6004 |
| siRNA 526 | 526 | 6016 |
| siRNA 528 | 528 | 6018 |
| siRNA 538 | 538 | 6028 |
| siRNA 541 | 541 | 6031 |
| siRNA 546 | 546 | 6036 |
| siRNA 562 | 562 | 6052 |
| siRNA 570 | 570 | 6060 |
| siRNA 573 | 573 | 6063 |
| siRNA 574 | 574 | 6064 |
| siRNA 575 | 575 | 6065 |
| siRNA 587 | 587 | 6077 |
| siRNA 589 | 589 | 6079 |
| siRNA 617 | 617 | 6107 |
| siRNA 633 | 633 | 6123 |
| siRNA 639 | 639 | 6129 |
| siRNA 643 | 643 | 6133 |
| siRNA 644 | 644 | 6134 |
| siRNA 647 | 647 | 6137 |
| siRNA 650 | 650 | 6140 |
| siRNA 651 | 651 | 6141 |
| siRNA 664 | 664 | 6154 |
| siRNA 671 | 671 | 6161 |
| siRNA 679 | 679 | 6169 |
| siRNA 680 | 680 | 6170 |
| siRNA 682 | 682 | 6172 |
| siRNA 685 | 685 | 6175 |
| siRNA 689 | 689 | 6179 |
| siRNA 690 | 690 | 6180 |
| siRNA 691 | 691 | 6181 |
| siRNA 692 | 692 | 6182 |
| siRNA 719 | 719 | 6209 |
| siRNA 736 | 736 | 6226 |
| siRNA 738 | 738 | 6228 |
| siRNA 739 | 739 | 6229 |
| siRNA 740 | 740 | 6230 |
| siRNA 743 | 743 | 6233 |
| siRNA 749 | 749 | 6239 |
| siRNA 750 | 750 | 6240 |
| siRNA 766 | 766 | 6256 |
| siRNA 783 | 783 | 6273 |
| siRNA 785 | 785 | 6275 |
| siRNA 786 | 786 | 6276 |
| siRNA 787 | 787 | 6277 |
| siRNA 788 | 788 | 6278 |
| siRNA 792 | 792 | 6282 |
| siRNA 796 | 796 | 6286 |
| siRNA 797 | 797 | 6287 |
| siRNA 813 | 813 | 6303 |
| siRNA 818 | 818 | 6308 |
| siRNA 819 | 819 | 6309 |
| siRNA 854 | 854 | 6344 |
| siRNA 877 | 877 | 6367 |
| siRNA 887 | 887 | 6377 |
| siRNA 888 | 888 | 6378 |
| siRNA 922 | 922 | 6412 |
| siRNA 930 | 930 | 6420 |
| siRNA 933 | 933 | 6423 |
| siRNA 934 | 934 | 6424 |
| siRNA 941 | 941 | 6431 |
| siRNA 942 | 942 | 6432 |
| siRNA 953 | 953 | 6443 |
| siRNA 954 | 954 | 6444 |
| siRNA 961 | 961 | 6451 |
| siRNA 967 | 967 | 6457 |
| siRNA 973 | 973 | 6463 |
| siRNA 999 | 999 | 6489 |
| siRNA 1000 | 1000 | 6490 |
| siRNA 1001 | 1001 | 6491 |
| siRNA 1007 | 1007 | 6497 |
| siRNA 1040 | 1040 | 6530 |
| siRNA 1053 | 1053 | 6543 |
| siRNA 1075 | 1075 | 6565 |
| siRNA 1084 | 1084 | 6574 |
| siRNA 1086 | 1086 | 6576 |
| siRNA 1087 | 1087 | 6577 |
| siRNA 1153 | 1153 | 6643 |
| siRNA 1156 | 1156 | 6646 |
| siRNA 1157 | 1157 | 6647 |
| siRNA 1159 | 1159 | 6649 |
| siRNA 1175 | 1175 | 6665 |
| siRNA 1196 | 1196 | 6686 |
| siRNA 1224 | 1224 | 6714 |
| siRNA 1255 | 1255 | 6745 |
| siRNA 1279 | 1279 | 6769 |
| siRNA 1280 | 1280 | 6770 |
| siRNA 1294 | 1294 | 6784 |
| siRNA 1296 | 1296 | 6786 |
| siRNA 1303 | 1303 | 6793 |
| siRNA 1343 | 1343 | 6833 |
| siRNA 1346 | 1346 | 6836 |
| siRNA 1347 | 1347 | 6837 |
| siRNA 1363 | 1363 | 6853 |
| siRNA 1398 | 1398 | 6888 |
| siRNA 1405 | 1405 | 6895 |
| siRNA 1406 | 1406 | 6896 |
| siRNA 1408 | 1408 | 6898 |
| siRNA 1430 | 1430 | 6920 |
| siRNA 1441 | 1441 | 6931 |
| siRNA 1442 | 1442 | 6932 |
| siRNA 1445 | 1445 | 6935 |
| siRNA 1449 | 1449 | 6939 |
| siRNA 1452 | 1452 | 6942 |
| siRNA 1453 | 1453 | 6943 |
| siRNA 1459 | 1459 | 6949 |
| siRNA 1460 | 1460 | 6950 |
| siRNA 1462 | 1462 | 6952 |
| siRNA 1466 | 1466 | 6956 |
| siRNA 1469 | 1469 | 6959 |
| siRNA 1470 | 1470 | 6960 |
| siRNA 1474 | 1474 | 6964 |
| siRNA 1498 | 1498 | 6988 |
| siRNA 1505 | 1505 | 6995 |
| siRNA 1510 | 1510 | 7000 |
| siRNA 1529 | 1529 | 7019 |
| siRNA 1533 | 1533 | 7023 |
| siRNA 1568 | 1568 | 7058 |
| siRNA 1569 | 1569 | 7059 |
| siRNA 1570 | 1570 | 7060 |
| siRNA 1572 | 1572 | 7062 |
| siRNA 1575 | 1575 | 7065 |
| siRNA 1587 | 1587 | 7077 |
| siRNA 1589 | 1589 | 7079 |
| siRNA 1596 | 1596 | 7086 |
| siRNA 1621 | 1621 | 7111 |
| siRNA 1622 | 1622 | 7112 |
| siRNA 1623 | 1623 | 7113 |
| siRNA 1633 | 1633 | 7123 |
| siRNA 1634 | 1634 | 7124 |
| siRNA 1635 | 1635 | 7125 |
| siRNA 1638 | 1638 | 7128 |
| siRNA 1651 | 1651 | 7141 |
| siRNA 1652 | 1652 | 7142 |
| siRNA 1654 | 1654 | 7144 |
| siRNA 1656 | 1656 | 7146 |
| siRNA 1683 | 1683 | 7173 |
| siRNA 1684 | 1684 | 7174 |
| siRNA 1699 | 1699 | 7189 |
| siRNA 1706 | 1706 | 7196 |
| siRNA 1708 | 1708 | 7198 |
| siRNA 1712 | 1712 | 7202 |
| siRNA 1720 | 1720 | 7210 |
| siRNA 1806 | 1806 | 7296 |
| siRNA 1814 | 1814 | 7304 |
| siRNA 1825 | 1825 | 7315 |
| siRNA 1826 | 1826 | 7316 |
| siRNA 1832 | 1832 | 7322 |
| siRNA 1846 | 1846 | 7336 |
| siRNA 1851 | 1851 | 7341 |
| siRNA 1861 | 1861 | 7351 |
| siRNA 1865 | 1865 | 7355 |
| siRNA 1909 | 1909 | 7399 |
| siRNA 1912 | 1912 | 7402 |
| siRNA 1917 | 1917 | 7407 |
| siRNA 1918 | 1918 | 7408 |
| siRNA 1922 | 1922 | 7412 |
| siRNA 1926 | 1926 | 7416 |
| siRNA 1927 | 1927 | 7417 |
| siRNA 1928 | 1928 | 7418 |
| siRNA 1929 | 1929 | 7419 |
| siRNA 1936 | 1936 | 7426 |
| siRNA 1937 | 1937 | 7427 |
| siRNA 1938 | 1938 | 7428 |
| siRNA 1940 | 1940 | 7430 |
| siRNA 1951 | 1951 | 7441 |
| siRNA 1980 | 1980 | 7470 |
| siRNA 1981 | 1981 | 7471 |
| siRNA 1986 | 1986 | 7476 |
| siRNA 1991 | 1991 | 7481 |
| siRNA 1992 | 1992 | 7482 |
| siRNA 1995 | 1995 | 7485 |
| siRNA 2000 | 2000 | 7490 |
| siRNA 2014 | 2014 | 7504 |
| siRNA 2020 | 2020 | 7510 |
| siRNA 2049 | 2049 | 7539 |
| siRNA 2051 | 2051 | 7541 |
| siRNA 2057 | 2057 | 7547 |
| siRNA 2062 | 2062 | 7552 |
| siRNA 2077 | 2077 | 7567 |
| siRNA 2112 | 2112 | 7602 |
| siRNA 2116 | 2116 | 7606 |
| siRNA 2148 | 2148 | 7638 |
| siRNA 2149 | 2149 | 7639 |
| siRNA 2150 | 2150 | 7640 |
| siRNA 2151 | 2151 | 7641 |
| siRNA 2154 | 2154 | 7644 |
| siRNA 2157 | 2157 | 7647 |
| siRNA 2158 | 2158 | 7648 |
| siRNA 2167 | 2167 | 7657 |
| siRNA 2168 | 2168 | 7658 |
| siRNA 2170 | 2170 | 7660 |
| siRNA 2172 | 2172 | 7662 |
| siRNA 2193 | 2193 | 7683 |
| siRNA 2202 | 2202 | 7692 |
| siRNA 2213 | 2213 | 7703 |
| siRNA 2244 | 2244 | 7734 |
| siRNA 2251 | 2251 | 7741 |
| siRNA 2252 | 2252 | 7742 |
| siRNA 2257 | 2257 | 7747 |
| siRNA 2286 | 2286 | 7776 |
| siRNA 2306 | 2306 | 7796 |
| siRNA 2307 | 2307 | 7797 |
| siRNA 2312 | 2312 | 7802 |
| siRNA 2313 | 2313 | 7803 |
| siRNA 2314 | 2314 | 7804 |
| siRNA 2318 | 2318 | 7808 |
| siRNA 2324 | 2324 | 7814 |
| siRNA 2328 | 2328 | 7818 |
| siRNA 2329 | 2329 | 7819 |
| siRNA 2332 | 2332 | 7822 |
| siRNA 2336 | 2336 | 7826 |
| siRNA 2339 | 2339 | 7829 |
| siRNA 2340 | 2340 | 7830 |
| siRNA 2356 | 2356 | 7846 |
| siRNA 2357 | 2357 | 7847 |
| siRNA 2360 | 2360 | 7850 |
| siRNA 2362 | 2362 | 7852 |
| siRNA 2364 | 2364 | 7854 |
| siRNA 2365 | 2365 | 7855 |
| siRNA 2366 | 2366 | 7856 |
| siRNA 2370 | 2370 | 7860 |
| siRNA 2403 | 2403 | 7893 |
| siRNA 2417 | 2417 | 7907 |
| siRNA 2418 | 2418 | 7908 |
| siRNA 2436 | 2436 | 7926 |
| siRNA 2472 | 2472 | 7962 |
| siRNA 2477 | 2477 | 7967 |
| siRNA 2480 | 2480 | 7970 |
| siRNA 2486 | 2486 | 7976 |
| siRNA 2528 | 2528 | 8018 |
| siRNA 2530 | 2530 | 8020 |
| siRNA 2531 | 2531 | 8021 |
| siRNA 2532 | 2532 | 8022 |
| siRNA 2568 | 2568 | 8058 |
| siRNA 2598 | 2598 | 8088 |
| siRNA 2620 | 2620 | 8110 |
| siRNA 2623 | 2623 | 8113 |
| siRNA 2631 | 2631 | 8121 |
| siRNA 2632 | 2632 | 8122 |
| siRNA 2633 | 2633 | 8123 |
| siRNA 2645 | 2645 | 8135 |
| siRNA 2646 | 2646 | 8136 |
| siRNA 2673 | 2673 | 8163 |
| siRNA 2679 | 2679 | 8169 |
| siRNA 2759 | 2759 | 8249 |
| siRNA 2760 | 2760 | 8250 |
| siRNA 2762 | 2762 | 8252 |
| siRNA 2764 | 2764 | 8254 |
| siRNA 2767 | 2767 | 8257 |
| siRNA 2815 | 2815 | 8305 |
| siRNA 2821 | 2821 | 8311 |
| siRNA 2828 | 2828 | 8318 |
| siRNA 2829 | 2829 | 8319 |
| siRNA 2830 | 2830 | 8320 |
| siRNA 2836 | 2836 | 8326 |
| siRNA 2841 | 2841 | 8331 |
| siRNA 2842 | 2842 | 8332 |
| siRNA 2847 | 2847 | 8337 |
| siRNA 2866 | 2866 | 8356 |
| siRNA 2883 | 2883 | 8373 |
| siRNA 2890 | 2890 | 8380 |
| siRNA 2898 | 2898 | 8388 |
| siRNA 2908 | 2908 | 8398 |
| siRNA 2928 | 2928 | 8418 |
| siRNA 2938 | 2938 | 8428 |
| siRNA 2939 | 2939 | 8429 |
| siRNA 2982 | 2982 | 8472 |
| siRNA 2986 | 2986 | 8476 |
| siRNA 2987 | 2987 | 8477 |
| siRNA 2998 | 2998 | 8488 |
| siRNA 3024 | 3024 | 8514 |
| siRNA 3025 | 3025 | 8515 |
| siRNA 3035 | 3035 | 8525 |
| siRNA 3038 | 3038 | 8528 |
| siRNA 3042 | 3042 | 8532 |
| siRNA 3053 | 3053 | 8543 |
| siRNA 3093 | 3093 | 8583 |
| siRNA 3094 | 3094 | 8584 |
| siRNA 3146 | 3146 | 8636 |
| siRNA 3164 | 3164 | 8654 |
| siRNA 3222 | 3222 | 8712 |
| siRNA 3231 | 3231 | 8721 |
| siRNA 3232 | 3232 | 8722 |
| siRNA 3238 | 3238 | 8728 |
| siRNA 3247 | 3247 | 8737 |
| siRNA 3259 | 3259 | 8749 |
| siRNA 3310 | 3310 | 8800 |
| siRNA 3333 | 3333 | 8823 |
| siRNA 3335 | 3335 | 8825 |
| siRNA 3339 | 3339 | 8829 |
| siRNA 3342 | 3342 | 8832 |
| siRNA 3350 | 3350 | 8840 |
| siRNA 3351 | 3351 | 8841 |
| siRNA 3352 | 3352 | 8842 |
| siRNA 3353 | 3353 | 8843 |
| siRNA 3357 | 3357 | 8847 |
| siRNA 3358 | 3358 | 8848 |
| siRNA 3364 | 3364 | 8854 |
| siRNA 3396 | 3396 | 8886 |
| siRNA 3405 | 3405 | 8895 |
| siRNA 3411 | 3411 | 8901 |
| siRNA 3412 | 3412 | 8902 |
| siRNA 3413 | 3413 | 8903 |
| siRNA 3417 | 3417 | 8907 |
| siRNA 3425 | 3425 | 8915 |
| siRNA 3436 | 3436 | 8926 |
| siRNA 3437 | 3437 | 8927 |
| siRNA 3444 | 3444 | 8934 |
| siRNA 3449 | 3449 | 8939 |
| siRNA 3450 | 3450 | 8940 |
| siRNA 3460 | 3460 | 8950 |
| siRNA 3461 | 3461 | 8951 |
| siRNA 3472 | 3472 | 8962 |
| siRNA 3487 | 3487 | 8977 |
| siRNA 3489 | 3489 | 8979 |
| siRNA 3490 | 3490 | 8980 |
| siRNA 3491 | 3491 | 8981 |
| siRNA 3500 | 3500 | 8990 |
| siRNA 3502 | 3502 | 8992 |
| siRNA 3540 | 3540 | 9030 |
| siRNA 3549 | 3549 | 9039 |
| siRNA 3560 | 3560 | 9050 |
| siRNA 3561 | 3561 | 9051 |
| siRNA 3564 | 3564 | 9054 |
| siRNA 3566 | 3566 | 9056 |
| siRNA 3586 | 3586 | 9076 |
| siRNA 3587 | 3587 | 9077 |
| siRNA 3602 | 3602 | 9092 |
| siRNA 3605 | 3605 | 9095 |
| siRNA 3639 | 3639 | 9129 |
| siRNA 3641 | 3641 | 9131 |
| siRNA 3677 | 3677 | 9167 |
| siRNA 3691 | 3691 | 9181 |
| siRNA 3703 | 3703 | 9193 |
| siRNA 3712 | 3712 | 9202 |
| siRNA 3746 | 3746 | 9236 |
| siRNA 3747 | 3747 | 9237 |
| siRNA 3748 | 3748 | 9238 |
| siRNA 3756 | 3756 | 9246 |
| siRNA 3792 | 3792 | 9282 |
| siRNA 3803 | 3803 | 9293 |
| siRNA 3810 | 3810 | 9300 |
| siRNA 3838 | 3838 | 9328 |
| siRNA 3869 | 3869 | 9359 |
| siRNA 3883 | 3883 | 9373 |
| siRNA 3922 | 3922 | 9412 |
| siRNA 3934 | 3934 | 9424 |
| siRNA 3943 | 3943 | 9433 |
| siRNA 3945 | 3945 | 9435 |
| siRNA 3970 | 3970 | 9460 |
| siRNA 3988 | 3988 | 9478 |
| siRNA 4040 | 4040 | 9530 |
| siRNA 4050 | 4050 | 9540 |
| siRNA 4051 | 4051 | 9541 |
| siRNA 4052 | 4052 | 9542 |
| siRNA 4053 | 4053 | 9543 |
| siRNA 4054 | 4054 | 9544 |
| siRNA 4058 | 4058 | 9548 |
| siRNA 4070 | 4070 | 9560 |
| siRNA 4112 | 4112 | 9602 |
| siRNA 4209 | 4209 | 9699 |
| siRNA 4210 | 4210 | 9700 |
| siRNA 4211 | 4211 | 9701 |
| siRNA 4214 | 4214 | 9704 |
| siRNA 4223 | 4223 | 9713 |
| siRNA 4224 | 4224 | 9714 |
| siRNA 4226 | 4226 | 9716 |
| siRNA 4235 | 4235 | 9725 |
| siRNA 4237 | 4237 | 9727 |
| siRNA 4238 | 4238 | 9728 |
| siRNA 4242 | 4242 | 9732 |
| siRNA 4245 | 4245 | 9735 |
| siRNA 4282 | 4282 | 9772 |
| siRNA 4290 | 4290 | 9780 |
| siRNA 4293 | 4293 | 9783 |
| siRNA 4294 | 4294 | 9784 |
| siRNA 4295 | 4295 | 9785 |
| siRNA 4296 | 4296 | 9786 |
| siRNA 4356 | 4356 | 9846 |
| siRNA 4368 | 4368 | 9858 |
| siRNA 4382 | 4382 | 9872 |
| siRNA 4388 | 4388 | 9878 |
| siRNA 4455 | 4455 | 9945 |
| siRNA 4460 | 4460 | 9950 |
| siRNA 4474 | 4474 | 9964 |
| siRNA 4475 | 4475 | 9965 |
| siRNA 4481 | 4481 | 9971 |
| siRNA 4482 | 4482 | 9972 |
| siRNA 4487 | 4487 | 9977 |
| siRNA 4488 | 4488 | 9978 |
| siRNA 4491 | 4491 | 9981 |
| siRNA 4521 | 4521 | 10011 |
| siRNA 4545 | 4545 | 10035 |
| siRNA 4547 | 4547 | 10037 |
| siRNA 4594 | 4594 | 10084 |
| siRNA 4595 | 4595 | 10085 |
| siRNA 4598 | 4598 | 10088 |
| siRNA 4629 | 4629 | 10119 |
| siRNA 4644 | 4644 | 10134 |
| siRNA 4705 | 4705 | 10195 |
| siRNA 4726 | 4726 | 10216 |
| siRNA 4739 | 4739 | 10229 |
| siRNA 4767 | 4767 | 10257 |
| siRNA 4800 | 4800 | 10290 |
| siRNA 4807 | 4807 | 10297 |
| siRNA 4852 | 4852 | 10342 |
| siRNA 4902 | 4902 | 10392 |
| siRNA 4903 | 4903 | 10393 |
| siRNA 4906 | 4906 | 10396 |
| siRNA 4910 | 4910 | 10400 |
| siRNA 4943 | 4943 | 10433 |
| siRNA 4975 | 4975 | 10465 |
| siRNA 4977 | 4977 | 10467 |
| siRNA 4981 | 4981 | 10471 |
| siRNA 4982 | 4982 | 10472 |
| siRNA 4983 | 4983 | 10473 |
| siRNA 4984 | 4984 | 10474 |
| siRNA 4989 | 4989 | 10479 |
| siRNA 4990 | 4990 | 10480 |
| siRNA 4993 | 4993 | 10483 |
| siRNA 4996 | 4996 | 10486 |
| siRNA 5000 | 5000 | 10490 |
| siRNA 5004 | 5004 | 10494 |
| siRNA 5017 | 5017 | 10507 |
| siRNA 5020 | 5020 | 10510 |
| siRNA 5027 | 5027 | 10517 |
| siRNA 5071 | 5071 | 10561 |
| siRNA 5072 | 5072 | 10562 |
| siRNA 5073 | 5073 | 10563 |
| siRNA 5100 | 5100 | 10590 |
| siRNA 5103 | 5103 | 10593 |
| siRNA 5118 | 5118 | 10608 |
| siRNA 5119 | 5119 | 10609 |
| siRNA 5120 | 5120 | 10610 |
| siRNA 5158 | 5158 | 10648 |
| siRNA 5164 | 5164 | 10654 |
| siRNA 5191 | 5191 | 10681 |
| siRNA 5194 | 5194 | 10684 |
| siRNA 5206 | 5206 | 10696 |
| siRNA 5207 | 5207 | 10697 |
| siRNA 5208 | 5208 | 10698 |
| siRNA 5210 | 5210 | 10700 |
| siRNA 5211 | 5211 | 10701 |
| siRNA 5214 | 5214 | 10704 |
| siRNA 5217 | 5217 | 10707 |
| siRNA 5280 | 5280 | 10770 |
| siRNA 5297 | 5297 | 10787 |
| siRNA 5334 | 5334 | 10824 |
| siRNA 5335 | 5335 | 10825 |
| siRNA 5340 | 5340 | 10830 |
| siRNA 5341 | 5341 | 10831 |
| siRNA 5343 | 5343 | 10833 |
| siRNA 5346 | 5346 | 10836 |
| siRNA 5349 | 5349 | 10839 |
| siRNA 5352 | 5352 | 10842 |
| siRNA 5374 | 5374 | 10864 |
| siRNA 5375 | 5375 | 10865 |
| siRNA 5392 | 5392 | 10882 |
| siRNA 5404 | 5404 | 10894 |
| siRNA 5408 | 5408 | 10898 |
| siRNA 5409 | 5409 | 10899 |
| siRNA 5443 | 5443 | 10933 |
| siRNA 5455 | 5455 | 10945 |
| siRNA 5478 | 5478 | 10968 |
| siRNA 5485 | 5485 | 10975 |

The siRNA sequences in subset D were also selected to have an off-target frequency of ≤20 human off-targets matched with 2 mismatches by antisense strand to yield subset E. Subset E includes 489 siRNAs whose base sequences are shown in Table 19.

TABLE 19
Subset E

| | Sense | Antisense |
|---|---|---|
| siRNA Name | SEQ ID NO: | SEQ ID NO: |
| siRNA 72 | 72 | 5562 |
| siRNA 106 | 106 | 5596 |
| siRNA 164 | 164 | 5654 |
| siRNA 224 | 224 | 5714 |
| siRNA 236 | 236 | 5726 |
| siRNA 266 | 266 | 5756 |
| siRNA 281 | 281 | 5771 |
| siRNA 282 | 282 | 5772 |
| siRNA 305 | 305 | 5795 |
| siRNA 313 | 313 | 5803 |
| siRNA 317 | 317 | 5807 |
| siRNA 336 | 336 | 5826 |
| siRNA 338 | 338 | 5828 |
| siRNA 342 | 342 | 5832 |
| siRNA 356 | 356 | 5846 |
| siRNA 381 | 381 | 5871 |
| siRNA 394 | 394 | 5884 |
| siRNA 403 | 403 | 5893 |
| siRNA 415 | 415 | 5905 |
| siRNA 457 | 457 | 5947 |
| siRNA 463 | 463 | 5953 |
| siRNA 482 | 482 | 5972 |
| siRNA 509 | 509 | 5999 |
| siRNA 510 | 510 | 6000 |
| siRNA 514 | 514 | 6004 |
| siRNA 526 | 526 | 6016 |
| siRNA 528 | 528 | 6018 |
| siRNA 538 | 538 | 6028 |
| siRNA 541 | 541 | 6031 |
| siRNA 546 | 546 | 6036 |
| siRNA 562 | 562 | 6052 |
| siRNA 570 | 570 | 6060 |
| siRNA 573 | 573 | 6063 |
| siRNA 574 | 574 | 6064 |
| siRNA 575 | 575 | 6065 |
| siRNA 587 | 587 | 6077 |
| siRNA 589 | 589 | 6079 |
| siRNA 617 | 617 | 6107 |
| siRNA 633 | 633 | 6123 |
| siRNA 639 | 639 | 6129 |
| siRNA 643 | 643 | 6133 |
| siRNA 644 | 644 | 6134 |
| siRNA 647 | 647 | 6137 |
| siRNA 650 | 650 | 6140 |
| siRNA 651 | 651 | 6141 |
| siRNA 664 | 664 | 6154 |
| siRNA 671 | 671 | 6161 |
| siRNA 679 | 679 | 6169 |
| siRNA 680 | 680 | 6170 |
| siRNA 682 | 682 | 6172 |
| siRNA 685 | 685 | 6175 |
| siRNA 689 | 689 | 6179 |
| siRNA 690 | 690 | 6180 |
| siRNA 691 | 691 | 6181 |
| siRNA 692 | 692 | 6182 |
| siRNA 719 | 719 | 6209 |
| siRNA 736 | 736 | 6226 |
| siRNA 738 | 738 | 6228 |
| siRNA 739 | 739 | 6229 |
| siRNA 740 | 740 | 6230 |
| siRNA 743 | 743 | 6233 |
| siRNA 749 | 749 | 6239 |
| siRNA 750 | 750 | 6240 |
| siRNA 766 | 766 | 6256 |
| siRNA 783 | 783 | 6273 |
| siRNA 785 | 785 | 6275 |
| siRNA 786 | 786 | 6276 |
| siRNA 787 | 787 | 6277 |
| siRNA 788 | 788 | 6278 |
| siRNA 792 | 792 | 6282 |
| siRNA 796 | 796 | 6286 |
| siRNA 797 | 797 | 6287 |
| siRNA 813 | 813 | 6303 |
| siRNA 818 | 818 | 6308 |
| siRNA 819 | 819 | 6309 |
| siRNA 854 | 854 | 6344 |
| siRNA 877 | 877 | 6367 |
| siRNA 887 | 887 | 6377 |
| siRNA 888 | 888 | 6378 |
| siRNA 922 | 922 | 6412 |
| siRNA 930 | 930 | 6420 |
| siRNA 933 | 933 | 6423 |
| siRNA 934 | 934 | 6424 |
| siRNA 941 | 941 | 6431 |
| siRNA 942 | 942 | 6432 |
| siRNA 953 | 953 | 6443 |
| siRNA 954 | 954 | 6444 |
| siRNA 961 | 961 | 6451 |
| siRNA 967 | 967 | 6457 |
| siRNA 973 | 973 | 6463 |
| siRNA 999 | 999 | 6489 |
| siRNA 1000 | 1000 | 6490 |
| siRNA 1001 | 1001 | 6491 |
| siRNA 1007 | 1007 | 6497 |
| siRNA 1040 | 1040 | 6530 |
| siRNA 1053 | 1053 | 6543 |
| siRNA 1075 | 1075 | 6565 |
| siRNA 1084 | 1084 | 6574 |
| siRNA 1086 | 1086 | 6576 |
| siRNA 1087 | 1087 | 6577 |
| siRNA 1153 | 1153 | 6643 |
| siRNA 1156 | 1156 | 6646 |
| siRNA 1157 | 1157 | 6647 |
| siRNA 1159 | 1159 | 6649 |
| siRNA 1175 | 1175 | 6665 |
| siRNA 1196 | 1196 | 6686 |
| siRNA 1224 | 1224 | 6714 |
| siRNA 1255 | 1255 | 6745 |
| siRNA 1279 | 1279 | 6769 |
| siRNA 1280 | 1280 | 6770 |
| siRNA 1294 | 1294 | 6784 |
| siRNA 1296 | 1296 | 6786 |
| siRNA 1303 | 1303 | 6793 |
| siRNA 1343 | 1343 | 6833 |
| siRNA 1346 | 1346 | 6836 |
| siRNA 1347 | 1347 | 6837 |
| siRNA 1363 | 1363 | 6853 |
| siRNA 1398 | 1398 | 6888 |
| siRNA 1405 | 1405 | 6895 |
| siRNA 1406 | 1406 | 6896 |
| siRNA 1408 | 1408 | 6898 |
| siRNA 1430 | 1430 | 6920 |
| siRNA 1441 | 1441 | 6931 |
| siRNA 1442 | 1442 | 6932 |
| siRNA 1445 | 1445 | 6935 |
| siRNA 1449 | 1449 | 6939 |
| siRNA 1452 | 1452 | 6942 |
| siRNA 1453 | 1453 | 6943 |
| siRNA 1459 | 1459 | 6949 |
| siRNA 1460 | 1460 | 6950 |
| siRNA 1462 | 1462 | 6952 |
| siRNA 1466 | 1466 | 6956 |
| siRNA 1469 | 1469 | 6959 |
| siRNA 1470 | 1470 | 6960 |
| siRNA 1474 | 1474 | 6964 |
| siRNA 1498 | 1498 | 6988 |
| siRNA 1505 | 1505 | 6995 |
| siRNA 1510 | 1510 | 7000 |
| siRNA 1529 | 1529 | 7019 |
| siRNA 1533 | 1533 | 7023 |
| siRNA 1568 | 1568 | 7058 |
| siRNA 1569 | 1569 | 7059 |
| siRNA 1570 | 1570 | 7060 |
| siRNA 1572 | 1572 | 7062 |
| siRNA 1575 | 1575 | 7065 |
| siRNA 1587 | 1587 | 7077 |
| siRNA 1589 | 1589 | 7079 |
| siRNA 1596 | 1596 | 7086 |
| siRNA 1621 | 1621 | 7111 |
| siRNA 1622 | 1622 | 7112 |
| siRNA 1623 | 1623 | 7113 |
| siRNA 1633 | 1633 | 7123 |
| siRNA 1634 | 1634 | 7124 |
| siRNA 1635 | 1635 | 7125 |
| siRNA 1638 | 1638 | 7128 |
| siRNA 1651 | 1651 | 7141 |
| siRNA 1652 | 1652 | 7142 |
| siRNA 1654 | 1654 | 7144 |
| siRNA 1656 | 1656 | 7146 |
| siRNA 1683 | 1683 | 7173 |
| siRNA 1684 | 1684 | 7174 |
| siRNA 1699 | 1699 | 7189 |
| siRNA 1706 | 1706 | 7196 |
| siRNA 1708 | 1708 | 7198 |
| siRNA 1712 | 1712 | 7202 |
| siRNA 1720 | 1720 | 7210 |
| siRNA 1806 | 1806 | 7296 |
| siRNA 1814 | 1814 | 7304 |
| siRNA 1825 | 1825 | 7315 |
| siRNA 1826 | 1826 | 7316 |
| siRNA 1832 | 1832 | 7322 |
| siRNA 1846 | 1846 | 7336 |
| siRNA 1851 | 1851 | 7341 |
| siRNA 1861 | 1861 | 7351 |
| siRNA 1865 | 1865 | 7355 |
| siRNA 1909 | 1909 | 7399 |
| siRNA 1912 | 1912 | 7402 |
| siRNA 1917 | 1917 | 7407 |
| siRNA 1918 | 1918 | 7408 |
| siRNA 1922 | 1922 | 7412 |
| siRNA 1926 | 1926 | 7416 |
| siRNA 1927 | 1927 | 7417 |
| siRNA 1928 | 1928 | 7418 |
| siRNA 1929 | 1929 | 7419 |
| siRNA 1936 | 1936 | 7426 |
| siRNA 1937 | 1937 | 7427 |
| siRNA 1938 | 1938 | 7428 |
| siRNA 1940 | 1940 | 7430 |
| siRNA 1951 | 1951 | 7441 |
| siRNA 1980 | 1980 | 7470 |
| siRNA 1981 | 1981 | 7471 |
| siRNA 1986 | 1986 | 7476 |
| siRNA 1991 | 1991 | 7481 |
| siRNA 1992 | 1992 | 7482 |
| siRNA 1995 | 1995 | 7485 |
| siRNA 2000 | 2000 | 7490 |
| siRNA 2014 | 2014 | 7504 |
| siRNA 2020 | 2020 | 7510 |
| siRNA 2049 | 2049 | 7539 |
| siRNA 2051 | 2051 | 7541 |
| siRNA 2057 | 2057 | 7547 |
| siRNA 2062 | 2062 | 7552 |
| siRNA 2077 | 2077 | 7567 |
| siRNA 2112 | 2112 | 7602 |
| siRNA 2116 | 2116 | 7606 |
| siRNA 2148 | 2148 | 7638 |
| siRNA 2149 | 2149 | 7639 |
| siRNA 2150 | 2150 | 7640 |
| siRNA 2151 | 2151 | 7641 |
| siRNA 2154 | 2154 | 7644 |
| siRNA 2157 | 2157 | 7647 |
| siRNA 2158 | 2158 | 7648 |
| siRNA 2167 | 2167 | 7657 |
| siRNA 2168 | 2168 | 7658 |
| siRNA 2170 | 2170 | 7660 |
| siRNA 2172 | 2172 | 7662 |
| siRNA 2193 | 2193 | 7683 |
| siRNA 2202 | 2202 | 7692 |
| siRNA 2213 | 2213 | 7703 |
| siRNA 2244 | 2244 | 7734 |
| siRNA 2251 | 2251 | 7741 |
| siRNA 2252 | 2252 | 7742 |
| siRNA 2257 | 2257 | 7747 |
| siRNA 2286 | 2286 | 7776 |
| siRNA 2306 | 2306 | 7796 |
| siRNA 2307 | 2307 | 7797 |
| siRNA 2312 | 2312 | 7802 |
| siRNA 2313 | 2313 | 7803 |
| siRNA 2314 | 2314 | 7804 |
| siRNA 2318 | 2318 | 7808 |
| siRNA 2324 | 2324 | 7814 |
| siRNA 2328 | 2328 | 7818 |
| siRNA 2329 | 2329 | 7819 |
| siRNA 2332 | 2332 | 7822 |
| siRNA 2336 | 2336 | 7826 |
| siRNA 2339 | 2339 | 7829 |
| siRNA 2340 | 2340 | 7830 |
| siRNA 2356 | 2356 | 7846 |
| siRNA 2357 | 2357 | 7847 |
| siRNA 2360 | 2360 | 7850 |
| siRNA 2362 | 2362 | 7852 |
| siRNA 2364 | 2364 | 7854 |
| siRNA 2365 | 2365 | 7855 |
| siRNA 2366 | 2366 | 7856 |
| siRNA 2370 | 2370 | 7860 |
| siRNA 2403 | 2403 | 7893 |
| siRNA 2417 | 2417 | 7907 |
| siRNA 2418 | 2418 | 7908 |
| siRNA 2436 | 2436 | 7926 |
| siRNA 2472 | 2472 | 7962 |
| siRNA 2477 | 2477 | 7967 |
| siRNA 2480 | 2480 | 7970 |
| siRNA 2486 | 2486 | 7976 |
| siRNA 2528 | 2528 | 8018 |
| siRNA 2530 | 2530 | 8020 |
| siRNA 2531 | 2531 | 8021 |
| siRNA 2532 | 2532 | 8022 |
| siRNA 2568 | 2568 | 8058 |
| siRNA 2598 | 2598 | 8088 |
| siRNA 2620 | 2620 | 8110 |
| siRNA 2623 | 2623 | 8113 |
| siRNA 2631 | 2631 | 8121 |
| siRNA 2632 | 2632 | 8122 |
| siRNA 2633 | 2633 | 8123 |
| siRNA 2645 | 2645 | 8135 |
| siRNA 2646 | 2646 | 8136 |
| siRNA 2673 | 2673 | 8163 |
| siRNA 2679 | 2679 | 8169 |
| siRNA 2759 | 2759 | 8249 |
| siRNA 2760 | 2760 | 8250 |
| siRNA 2762 | 2762 | 8252 |
| siRNA 2764 | 2764 | 8254 |
| siRNA 2767 | 2767 | 8257 |
| siRNA 2815 | 2815 | 8305 |
| siRNA 2821 | 2821 | 8311 |
| siRNA 2828 | 2828 | 8318 |
| siRNA 2829 | 2829 | 8319 |
| siRNA 2830 | 2830 | 8320 |
| siRNA 2836 | 2836 | 8326 |
| siRNA 2841 | 2841 | 8331 |
| siRNA 2842 | 2842 | 8332 |
| siRNA 2847 | 2847 | 8337 |
| siRNA 2866 | 2866 | 8356 |
| siRNA 2883 | 2883 | 8373 |
| siRNA 2890 | 2890 | 8380 |
| siRNA 2898 | 2898 | 8388 |
| siRNA 2908 | 2908 | 8398 |
| siRNA 2928 | 2928 | 8418 |
| siRNA 2938 | 2938 | 8428 |
| siRNA 2939 | 2939 | 8429 |
| siRNA 2982 | 2982 | 8472 |
| siRNA 2986 | 2986 | 8476 |
| siRNA 2987 | 2987 | 8477 |
| siRNA 2998 | 2998 | 8488 |
| siRNA 3024 | 3024 | 8514 |
| siRNA 3025 | 3025 | 8515 |
| siRNA 3035 | 3035 | 8525 |
| siRNA 3038 | 3038 | 8528 |
| siRNA 3042 | 3042 | 8532 |
| siRNA 3053 | 3053 | 8543 |
| siRNA 3093 | 3093 | 8583 |
| siRNA 3094 | 3094 | 8584 |
| siRNA 3146 | 3146 | 8636 |
| siRNA 3164 | 3164 | 8654 |
| siRNA 3222 | 3222 | 8712 |
| siRNA 3231 | 3231 | 8721 |
| siRNA 3232 | 3232 | 8722 |
| siRNA 3238 | 3238 | 8728 |
| siRNA 3247 | 3247 | 8737 |
| siRNA 3259 | 3259 | 8749 |
| siRNA 3310 | 3310 | 8800 |
| siRNA 3333 | 3333 | 8823 |
| siRNA 3335 | 3335 | 8825 |
| siRNA 3339 | 3339 | 8829 |
| siRNA 3342 | 3342 | 8832 |
| siRNA 3350 | 3350 | 8840 |
| siRNA 3351 | 3351 | 8841 |
| siRNA 3352 | 3352 | 8842 |
| siRNA 3353 | 3353 | 8843 |
| siRNA 3357 | 3357 | 8847 |
| siRNA 3358 | 3358 | 8848 |
| siRNA 3364 | 3364 | 8854 |
| siRNA 3396 | 3396 | 8886 |
| siRNA 3405 | 3405 | 8895 |
| siRNA 3411 | 3411 | 8901 |
| siRNA 3412 | 3412 | 8902 |
| siRNA 3413 | 3413 | 8903 |
| siRNA 3417 | 3417 | 8907 |
| siRNA 3425 | 3425 | 8915 |
| siRNA 3436 | 3436 | 8926 |
| siRNA 3437 | 3437 | 8927 |
| siRNA 3444 | 3444 | 8934 |
| siRNA 3449 | 3449 | 8939 |
| siRNA 3450 | 3450 | 8940 |
| siRNA 3460 | 3460 | 8950 |
| siRNA 3461 | 3461 | 8951 |
| siRNA 3472 | 3472 | 8962 |
| siRNA 3487 | 3487 | 8977 |
| siRNA 3489 | 3489 | 8979 |
| siRNA 3490 | 3490 | 8980 |
| siRNA 3491 | 3491 | 8981 |
| siRNA 3500 | 3500 | 8990 |
| siRNA 3502 | 3502 | 8992 |
| siRNA 3540 | 3540 | 9030 |
| siRNA 3549 | 3549 | 9039 |
| siRNA 3560 | 3560 | 9050 |
| siRNA 3561 | 3561 | 9051 |
| siRNA 3564 | 3564 | 9054 |
| siRNA 3566 | 3566 | 9056 |
| siRNA 3586 | 3586 | 9076 |
| siRNA 3587 | 3587 | 9077 |
| siRNA 3602 | 3602 | 9092 |
| siRNA 3605 | 3605 | 9095 |
| siRNA 3639 | 3639 | 9129 |
| siRNA 3641 | 3641 | 9131 |
| siRNA 3677 | 3677 | 9167 |
| siRNA 3691 | 3691 | 9181 |
| siRNA 3703 | 3703 | 9193 |
| siRNA 3712 | 3712 | 9202 |
| siRNA 3746 | 3746 | 9236 |
| siRNA 3747 | 3747 | 9237 |
| siRNA 3748 | 3748 | 9238 |
| siRNA 3756 | 3756 | 9246 |
| siRNA 3792 | 3792 | 9282 |
| siRNA 3803 | 3803 | 9293 |
| siRNA 3810 | 3810 | 9300 |
| siRNA 3838 | 3838 | 9328 |
| siRNA 3869 | 3869 | 9359 |
| siRNA 3883 | 3883 | 9373 |
| siRNA 3922 | 3922 | 9412 |
| siRNA 3934 | 3934 | 9424 |
| siRNA 3943 | 3943 | 9433 |
| siRNA 3945 | 3945 | 9435 |
| siRNA 3970 | 3970 | 9460 |
| siRNA 3988 | 3988 | 9478 |
| siRNA 4040 | 4040 | 9530 |
| siRNA 4050 | 4050 | 9540 |
| siRNA 4051 | 4051 | 9541 |
| siRNA 4052 | 4052 | 9542 |
| siRNA 4053 | 4053 | 9543 |
| siRNA 4054 | 4054 | 9544 |
| siRNA 4058 | 4058 | 9548 |
| siRNA 4070 | 4070 | 9560 |
| siRNA 4112 | 4112 | 9602 |
| siRNA 4209 | 4209 | 9699 |
| siRNA 4210 | 4210 | 9700 |
| siRNA 4211 | 4211 | 9701 |
| siRNA 4214 | 4214 | 9704 |
| siRNA 4223 | 4223 | 9713 |
| siRNA 4224 | 4224 | 9714 |
| siRNA 4226 | 4226 | 9716 |
| siRNA 4235 | 4235 | 9725 |
| siRNA 4237 | 4237 | 9727 |
| siRNA 4238 | 4238 | 9728 |
| siRNA 4242 | 4242 | 9732 |
| siRNA 4245 | 4245 | 9735 |
| siRNA 4282 | 4282 | 9772 |
| siRNA 4290 | 4290 | 9780 |
| siRNA 4293 | 4293 | 9783 |
| siRNA 4294 | 4294 | 9784 |
| siRNA 4295 | 4295 | 9785 |
| siRNA 4296 | 4296 | 9786 |
| siRNA 4356 | 4356 | 9846 |
| siRNA 4368 | 4368 | 9858 |
| siRNA 4382 | 4382 | 9872 |
| siRNA 4388 | 4388 | 9878 |
| siRNA 4455 | 4455 | 9945 |
| siRNA 4460 | 4460 | 9950 |
| siRNA 4474 | 4474 | 9964 |
| siRNA 4475 | 4475 | 9965 |
| siRNA 4481 | 4481 | 9971 |
| siRNA 4482 | 4482 | 9972 |
| siRNA 4487 | 4487 | 9977 |
| siRNA 4488 | 4488 | 9978 |
| siRNA 4491 | 4491 | 9981 |
| siRNA 4521 | 4521 | 10011 |
| siRNA 4545 | 4545 | 10035 |
| siRNA 4547 | 4547 | 10037 |
| siRNA 4594 | 4594 | 10084 |
| siRNA 4595 | 4595 | 10085 |
| siRNA 4598 | 4598 | 10088 |
| siRNA 4629 | 4629 | 10119 |
| siRNA 4644 | 4644 | 10134 |
| siRNA 4705 | 4705 | 10195 |
| siRNA 4726 | 4726 | 10216 |
| siRNA 4739 | 4739 | 10229 |
| siRNA 4767 | 4767 | 10257 |
| siRNA 4800 | 4800 | 10290 |
| siRNA 4807 | 4807 | 10297 |
| siRNA 4852 | 4852 | 10342 |
| siRNA 4902 | 4902 | 10392 |
| siRNA 4903 | 4903 | 10393 |
| siRNA 4906 | 4906 | 10396 |
| siRNA 4910 | 4910 | 10400 |
| siRNA 4943 | 4943 | 10433 |
| siRNA 4975 | 4975 | 10465 |
| siRNA 4977 | 4977 | 10467 |
| siRNA 4981 | 4981 | 10471 |
| siRNA 4982 | 4982 | 10472 |
| siRNA 4983 | 4983 | 10473 |
| siRNA 4984 | 4984 | 10474 |
| siRNA 4989 | 4989 | 10479 |
| siRNA 4990 | 4990 | 10480 |
| siRNA 4993 | 4993 | 10483 |
| siRNA 4996 | 4996 | 10486 |
| siRNA 5000 | 5000 | 10490 |
| siRNA 5004 | 5004 | 10494 |
| siRNA 5017 | 5017 | 10507 |
| siRNA 5020 | 5020 | 10510 |
| siRNA 5027 | 5027 | 10517 |
| siRNA 5071 | 5071 | 10561 |
| siRNA 5072 | 5072 | 10562 |
| siRNA 5073 | 5073 | 10563 |
| siRNA 5100 | 5100 | 10590 |
| siRNA 5103 | 5103 | 10593 |
| siRNA 5118 | 5118 | 10608 |
| siRNA 5119 | 5119 | 10609 |
| siRNA 5120 | 5120 | 10610 |
| siRNA 5158 | 5158 | 10648 |
| siRNA 5164 | 5164 | 10654 |
| siRNA 5191 | 5191 | 10681 |
| siRNA 5194 | 5194 | 10684 |
| siRNA 5206 | 5206 | 10696 |
| siRNA 5207 | 5207 | 10697 |
| siRNA 5208 | 5208 | 10698 |
| siRNA 5210 | 5210 | 10700 |
| siRNA 5211 | 5211 | 10701 |
| siRNA 5214 | 5214 | 10704 |
| siRNA 5217 | 5217 | 10707 |
| siRNA 5280 | 5280 | 10770 |
| siRNA 5297 | 5297 | 10787 |
| siRNA 5334 | 5334 | 10824 |
| siRNA 5335 | 5335 | 10825 |
| siRNA 5340 | 5340 | 10830 |
| siRNA 5341 | 5341 | 10831 |
| siRNA 5343 | 5343 | 10833 |
| siRNA 5346 | 5346 | 10836 |
| siRNA 5349 | 5349 | 10839 |
| siRNA 5352 | 5352 | 10842 |
| siRNA 5374 | 5374 | 10864 |
| siRNA 5375 | 5375 | 10865 |
| siRNA 5392 | 5392 | 10882 |
| siRNA 5404 | 5404 | 10894 |
| siRNA 5408 | 5408 | 10898 |
| siRNA 5409 | 5409 | 10899 |
| siRNA 5443 | 5443 | 10933 |
| siRNA 5455 | 5455 | 10945 |
| siRNA 5478 | 5478 | 10968 |
| siRNA 5485 | 5485 | 10975 |

Therapeutic siRNAs were designed to target human SOS2 as described above and, in some cases, the SOS2 sequences of two toxicology-relevant species, in this case, the non-human primate (NHP) cynomolgus monkey and the mouse. The siRNAs included in subset F had the following characteristics: Cross-reactivity with 19mer in human SOS2 mRNA, with 17mer/19mer in cynomolgus monkey SOS2, and with 17mer/19mer in mouse SOS2; Specificity: Subset A siRNAs for human, specificity category AS2 or better and SS3 or better for cynomolgus monkey, and specificity category AS2 or better and SS3 or better for mouse.

Subset F includes 20 siRNAs whose base sequences are shown in Table 20.

TABLE 20
Subset F

| | Sense | Antisense |
|---|---|---|
| siRNA Name | SEQ ID NO: | SEQ ID NO: |
| siRNA 743 | 743 | 6233 |
| siRNA 744 | 744 | 6234 |
| siRNA 1036 | 1036 | 6526 |
| siRNA 1056 | 1056 | 6546 |
| siRNA 1178 | 1178 | 6668 |
| siRNA 1521 | 1521 | 7011 |
| siRNA 2148 | 2148 | 7638 |
| siRNA 2151 | 2151 | 7641 |
| siRNA 2152 | 2152 | 7642 |
| siRNA 2158 | 2158 | 7648 |
| siRNA 2619 | 2619 | 8109 |
| siRNA 2882 | 2882 | 8372 |
| siRNA 2883 | 2883 | 8373 |
| siRNA 2884 | 2884 | 8374 |
| siRNA 3042 | 3042 | 8532 |
| siRNA 3305 | 3305 | 8795 |
| siRNA 3969 | 3969 | 9459 |
| siRNA 4216 | 4216 | 9706 |
| siRNA 4305 | 4305 | 9795 |
| siRNA 4309 | 4309 | 9799 |

In some cases, the sense strand of any of the siRNAs of subset F comprises siRNA with a particular modification pattern. In this modification pattern, position 9 counting from the 5′ end of the of the sense strand is has the 2′F modification. If position 9 of the sense strand is a pyrimidine, then all purines in the sense strand have the 2′OMe modification. If position 9 is the only pyrimidine between positions 5 and 11 of the sense stand, then position 9 is the only position with the 2′F modification in the sense strand. If position 9 and only one other base between positions 5 and 11 of the sense strand are pyrimidines, then both of these pyrimidines are the only two positions with the 2′F modification in the sense strand. If position 9 and only two other bases between positions 5 and 11 of the sense strand are pyrimidines, and those two other pyrimidines are in adjacent positions so that there would be not three 2′F modifications in a row, then any combination of 2′F modifications can be made that give three 2′F modifications in total. If there are >2 pyrimidines between positions 5 and 11 of the sense strand, then all combinations of pyrimidines having the 2′F modification are allowed that have three to five 2′F modifications in total, provided that the sense strand does not have three 2′F modifications in a row.

If position 9 of the sense strand is a purine, then all purines in the sense strand have the 2′OMe modification. If position 9 is the only purine between positions 5 and 11 of the sense stand, then position 9 is the only position with the 2′F modification in the sense strand. If position 9 and only one other base between positions 5 and 11 of the sense strand are purines, then both of these purines are the only two positions with the 2′F modification in the sense strand. If position 9 and only two other bases between positions 5 and 11 of the sense strand are purines, and those two other purines are in adjacent positions so that there would be not three 2′F modifications in a row, then any combination of 2′F modifications can be made that give three 2′F modifications in total. If there are >2 purines between positions 5 and 11 of the sense strand, then all combinations of purines having the 2′F modification are allowed that have three to five 2′F modifications in total, provided that the sense strand does not have three 2′F modifications in a row.

In some cases, position 9 of the sense strand can be a 2′deoxy. In these cases, 2′F and 2′OMe modifications may occur at the other positions of the sense strand.

In some cases, the sense strand of any of the siRNAs of subset F comprises a modification pattern which conforms to these sense strand rules (Table 21).

In some cases, the antisense strand of any of the siRNAs of subset F comprise a modification or modification pattern. Some such examples are included in Table 21. Table 22 includes some additional sense strand modifications of the siRNAs in Table 20. The siRNAs in subset F may comprise any other modification pattern(s).

TABLE 21
subset G

| SEQ | Sense strand sequence | SEQ | Antisense strand sequence |
|---|---|---|---|
| ID NO: | (5′-3′) | ID NO: | (5′-3′) |
| 10981 | uuuaaUfaUfCfcggcauuauasusu | 11001 | usAfsuAfaUfgCfcGfgAfuAfuUfaAfasusu |
| 10982 | uuaaUfaUfcCfggcauuaugasusu | 11002 | usCfsaUfaAfuGfcCfgGfaUfaUfuAfasusu |
| 10983 | uucagaUfaUfaCfaugaauuasusu | 11003 | usAfsaUfuCfaUfgUfaUfaUfcUfgAfasusu |
| 10984 | cugugaaaCfUfuuuagguuasusu | 11004 | usAfsaCfcUfaAfaAfgUfuUfcAfcAfgsusu |
| 10985 | acauuAfucAfcAfggacauuasusu | 11005 | usAfsaUfgUfcCfuGfuGfaUfaAfuGfususu |
| 10986 | aauuAfAfGfAfAfgcaaacaccasusu | 11006 | usGfsgUfgUfuUfgCfuUfcUfuAfaUfususu |
| 10987 | auccCfaaUfUfUfUfguucguaasusu | 11007 | usUfsaCfgAfaCfaAfaAfuUfgGfgAfususu |
| 10988 | ccaauuuuGfuucguacuuasusu | 11008 | usAfsaGfuAfcGfaAfcAfaAfaUfuGfgsusu |
| 10989 | caauUfUfugUfUfcguacuuuasusu | 11009 | usAfsaAfgUfaCfgAfaCfaAfaAfuUfgsusu |
| 10990 | uguuCfgUfaCfUfuuucuuacasusu | 11010 | usGfsuAfaGfaAfaAfgUfaCfgAfaCfasusu |
| 10991 | ugacaCfUfUfCfaUfccaauagasusu | 11011 | usCfsuAfuUfgGfaUfgAfaGfuGfuCfasusu |
| 10992 | uugaaUfaaUfUfucaauggcasusu | 11012 | usGfscCfaUfuGfaAfaUfuAfuUfcAfasusu |
| 10993 | ugaaUfaaUfUfUfCfaauggcgasusu | 11013 | usCfsgCfcAfuUfgAfaAfuUfaUfuCfasusu |
| 10994 | gaauaaUfUfUfCfaauggcguasusu | 11014 | usAfscGfcCfaUfuGfaAfaUfuAfuUfcsusu |
| 10995 | aacuUfaagUfCfaaucaaucasusu | 11015 | usGfsaUfuGfaUfuGfaCfuUfaAfgUfususu |
| 10996 | acagaUfUfaUfUfuguucaacasusu | 11016 | usGfsuUfgAfaCfaAfaUfaAfuCfuGfususu |
| 10997 | aacacuuuAfuAfaacugucasusu | 11017 | usGfsaCfaGfuUfuAfuAfaAfgUfgUfususu |
| 10998 | aaagAfcuuAfcAfaacgggaasusu | 11018 | usUfscCfcGfuUfuGfuAfaGfuCfuUfususu |
| 10999 | auguAfGfucAfuugacacugasusu | 11019 | usCfsaGfuGfuCfaAfuGfaCfuAfcAfususu |
| 11000 | agucAfuuGfAfcAfcuggaauasusu | 11020 | usAfsuUfcCfaGfuGfuCfaAfuGfaCfususu |

TABLE 22
Subset H

| SEQ | Sense strand sequence | SEQ | Antisense strand sequence |
|---|---|---|---|
| ID NO: | (5′-3′) | ID NO: | (5′-3′) |
| 11021 | uuuaaUfauCfCfggcauuauasusu | 11001 | usAfsuAfaUfgCfcGfgAfuAfuUfaAfasusu |
| 11022 | uuaaUfauCfCfggcauuaugasusu | 11002 | usCfsaUfaAfuGfcCfgGfaUfaUfuAfasusu |
| 11023 | cugugaaaCfuUfuuagguuasusu | 11004 | usAfsaCfcUfaAfaAfgUfuUfcAfcAfgsusu |
| 11024 | aauuaaGfaAfGfcaaacaccasusu | 11006 | usGfsgUfgUfuUfgCfuUfcUfuAfaUfususu |
| 11025 | aauuaaGfAfAfGfcaaacaccasusu | 11006 | usGfsgUfgUfuUfgCfuUfcUfuAfaUfususu |
| 11026 | aauuaAfgaAfGfcaaacaccasusu | 11006 | usGfsgUfgUfuUfgCfuUfcUfuAfaUfususu |
| 11027 | aauuaAfgAfAfgcaaacaccasusu | 11006 | usGfsgUfgUfuUfgCfuUfcUfuAfaUfususu |
| 11028 | aauuaAfGfaAfgcaaacaccasusu | 11006 | usGfsgUfgUfuUfgCfuUfcUfuAfaUfususu |
| 11029 | aauuaAfGfaAfGfcaaacaccasusu | 11006 | usGfsgUfgUfuUfgCfuUfcUfuAfaUfususu |
| 11030 | aauuaAfGfAfAfgcaaacaccasusu | 11006 | usGfsgUfgUfuUfgCfuUfcUfuAfaUfususu |
| 11031 | aauuaAfGfAfAfGfcaaacaccasusu | 11006 | usGfsgUfgUfuUfgCfuUfcUfuAfaUfususu |
| 11032 | aauuAfagaAfGfcaaacaccasusu | 11006 | usGfsgUfgUfuUfgCfuUfcUfuAfaUfususu |
| 11033 | aauuAfagAfAfgcaaacaccasusu | 11006 | usGfsgUfgUfuUfgCfuUfcUfuAfaUfususu |
| 11034 | aauuAfaGfaAfgcaaacaccasusu | 11006 | usGfsgUfgUfuUfgCfuUfcUfuAfaUfususu |
| 11035 | aauuAfaGfaAfGfcaaacaccasusu | 11006 | usGfsgUfgUfuUfgCfuUfcUfuAfaUfususu |
| 11036 | aauuAfaGfAfAfGfcaaacaccasusu | 11006 | usGfsgUfgUfuUfgCfuUfcUfuAfaUfususu |
| 11037 | aauuAfAfgaAfgcaaacaccasusu | 11006 | usGfsgUfgUfuUfgCfuUfcUfuAfaUfususu |
| 11038 | aauuAfAfgaAfGfcaaacaccasusu | 11006 | usGfsgUfgUfuUfgCfuUfcUfuAfaUfususu |
| 11039 | aauuAfAfgAfAfgcaaacaccasusu | 11006 | usGfsgUfgUfuUfgCfuUfcUfuAfaUfususu |
| 11040 | aucccaaUfUfuUfguucguaasusu | 11007 | usUfsaCfgAfaCfaAfaAfuUfgGfgAfususu |
| 11041 | aucccaaUfUfUfUfguucguaasusu | 11007 | usUfsaCfgAfaCfaAfaAfuUfgGfgAfususu |
| 11042 | auccCfaauUfuUfguucguaasusu | 11007 | usUfsaCfgAfaCfaAfaAfuUfgGfgAfususu |
| 11043 | auccCfaauUfUfuguucguaasusu | 11007 | usUfsaCfgAfaCfaAfaAfuUfgGfgAfususu |
| 11044 | auccCfaaUfUfuuguucguaasusu | 11007 | usUfsaCfgAfaCfaAfaAfuUfgGfgAfususu |
| 11045 | auccCfaaUfUfuUfguucguaasusu | 11007 | usUfsaCfgAfaCfaAfaAfuUfgGfgAfususu |
| 11046 | caauuuUfgUfuCfguacuuuasusu | 11009 | usAfsaAfgUfaCfgAfaCfaAfaAfuUfgsusu |
| 11047 | caauuuUfgUfUfcguacuuuasusu | 11009 | usAfsaAfgUfaCfgAfaCfaAfaAfuUfgsusu |
| 11048 | caauuUfugUfuCfguacuuuasusu | 11009 | usAfsaAfgUfaCfgAfaCfaAfaAfuUfgsusu |
| 11049 | caauuUfugUfUfcguacuuuasusu | 11009 | usAfsaAfgUfaCfgAfaCfaAfaAfuUfgsusu |
| 11050 | caauuUfUfgUfucguacuuuasusu | 11009 | usAfsaAfgUfaCfgAfaCfaAfaAfuUfgsusu |
| 11051 | caauuUfUfgUfuCfguacuuuasusu | 11009 | usAfsaAfgUfaCfgAfaCfaAfaAfuUfgsusu |
| 11052 | caauuUfUfgUfUfcguacuuuasusu | 11009 | usAfsaAfgUfaCfgAfaCfaAfaAfuUfgsusu |
| 11053 | caauUfuugUfuCfguacuuuasusu | 11009 | usAfsaAfgUfaCfgAfaCfaAfaAfuUfgsusu |
| 11054 | caauUfuugUfUfcguacuuuasusu | 11009 | usAfsaAfgUfaCfgAfaCfaAfaAfuUfgsusu |
| 11055 | caauUfuUfgUfucguacuuuasusu | 11009 | usAfsaAfgUfaCfgAfaCfaAfaAfuUfgsusu |
| 11056 | caauUfuUfgUfuCfguacuuuasusu | 11009 | usAfsaAfgUfaCfgAfaCfaAfaAfuUfgsusu |
| 11057 | caauUfuUfgUfUfcguacuuuasusu | 11009 | usAfsaAfgUfaCfgAfaCfaAfaAfuUfgsusu |
| 11058 | caauUfUfugUfucguacuuuasusu | 11009 | usAfsaAfgUfaCfgAfaCfaAfaAfuUfgsusu |
| 11059 | caauUfUfugUfuCfguacuuuasusu | 11009 | usAfsaAfgUfaCfgAfaCfaAfaAfuUfgsusu |
| 11060 | uguucgUfaCfuUfuucuuacasusu | 11010 | usGfsuAfaGfaAfaAfgUfaCfgAfaCfasusu |
| 11061 | uguucgUfaCfUfuuucuuacasusu | 11010 | usGfsuAfaGfaAfaAfgUfaCfgAfaCfasusu |
| 11062 | uguuCfguaCfuUfuucuuacasusu | 11010 | usGfsuAfaGfaAfaAfgUfaCfgAfaCfasusu |
| 11063 | uguuCfguaCfUfuuucuuacasusu | 11010 | usGfsuAfaGfaAfaAfgUfaCfgAfaCfasusu |
| 11064 | uguuCfgUfaCfuuuucuuacasusu | 11010 | usGfsuAfaGfaAfaAfgUfaCfgAfaCfasusu |
| 11065 | uguuCfgUfaCfuUfuucuuacasusu | 11010 | usGfsuAfaGfaAfaAfgUfaCfgAfaCfasusu |
| 11066 | ugacacuUfCfaUfccaauagasusu | 11011 | usCfsuAfuUfgGfaUfgAfaGfuGfuCfasusu |
| 11067 | ugacacUfuCfaUfccaauagasusu | 11011 | usCfsuAfuUfgGfaUfgAfaGfuGfuCfasusu |
| 11068 | ugacaCfuuCfaUfccaauagasusu | 11011 | usCfsuAfuUfgGfaUfgAfaGfuGfuCfasusu |
| 11069 | ugacaCfuUfCfauccaauagasusu | 11011 | usCfsuAfuUfgGfaUfgAfaGfuGfuCfasusu |
| 11070 | ugacaCfuUfCfaUfccaauagasusu | 11011 | usCfsuAfuUfgGfaUfgAfaGfuGfuCfasusu |
| 11071 | ugacaCfUfuCfauccaauagasusu | 11011 | usCfsuAfuUfgGfaUfgAfaGfuGfuCfasusu |
| 11072 | ugacaCfUfuCfaUfccaauagasusu | 11011 | usCfsuAfuUfgGfaUfgAfaGfuGfuCfasusu |
| 11073 | ugacaCfUfUfCfauccaauagasusu | 11011 | usCfsuAfuUfgGfaUfgAfaGfuGfuCfasusu |
| 11074 | uugaaUfaaUfuUfcaauggcasusu | 11012 | usGfscCfaUfuGfaAfaUfuAfuUfcAfasusu |
| 11075 | ugaauaaUfUfuCfaauggcgasusu | 11013 | usCfsgCfcAfuUfgAfaAfuUfaUfuCfasusu |
| 11076 | ugaauaaUfUfUfCfaauggcgasusu | 11013 | usCfsgCfcAfuUfgAfaAfuUfaUfuCfasusu |
| 11077 | ugaaUfaauUfuCfaauggcgasusu | 11013 | usCfsgCfcAfuUfgAfaAfuUfaUfuCfasusu |
| 11078 | ugaaUfaauUfUfcaauggcgasusu | 11013 | usCfsgCfcAfuUfgAfaAfuUfaUfuCfasusu |
| 11079 | ugaaUfaaUfUfucaauggcgasusu | 11013 | usCfsgCfcAfuUfgAfaAfuUfaUfuCfasusu |
| 11080 | ugaaUfaaUfUfuCfaauggcgasusu | 11013 | usCfsgCfcAfuUfgAfaAfuUfaUfuCfasusu |
| 11081 | gaauaaUfuUfCfaauggcguasusu | 11014 | usAfscGfcCfaUfuGfaAfaUfuAfuUfcsusu |
| 11082 | acagauUfaUfuUfguucaacasusu | 11016 | usGfsuUfgAfaCfaAfaUfaAfuCfuGfususu |
| 11083 | acagauUfaUfUfuguucaacasusu | 11016 | usGfsuUfgAfaCfaAfaUfaAfuCfuGfususu |
| 11084 | acagaUfuaUfuUfguucaacasusu | 11016 | usGfsuUfgAfaCfaAfaUfaAfuCfuGfususu |
| 11085 | acagaUfuaUfUfuguucaacasusu | 11016 | usGfsuUfgAfaCfaAfaUfaAfuCfuGfususu |
| 11086 | acagaUfUfaUfuuguucaacasusu | 11016 | usGfsuUfgAfaCfaAfaUfaAfuCfuGfususu |
| 11087 | acagaUfUfaUfuUfguucaacasusu | 11016 | usGfsuUfgAfaCfaAfaUfaAfuCfuGfususu |
| 11088 | agucauuGfAfcAfcuggaauasusu | 11020 | usAfsuUfcCfaGfuGfuCfaAfuGfaCfususu |
| 11089 | agucAfuugAfcAfcuggaauasusu | 11020 | usAfsuUfcCfaGfuGfuCfaAfuGfaCfususu |
| 11090 | agucAfuuGfAfcacuggaauasusu | 11020 | usAfsuUfcCfaGfuGfuCfaAfuGfaCfususu |

Therapeutic siRNAs were designed to target human SOS2 as described above and, in some cases, the SOS2 sequence of at least one toxicology-relevant species, in this case, the non-human primate (NHP) cynomolgus monkey. The siRNAs included in subset I had the following characteristics: Cross-reactivity with 19mer in human SOS2 mRNA and with 17mer/19mer in cynomolgus monkey SOS2; Specificity category: Subset E siRNAs for human with a specificity category of AS1 or better and SS2 or better, specificity category AS2 or better and SS3 or better for cynomolgus monkey, and specificity category AS2 or better and SS3 or better for mouse.

Subset I includes 25 siRNAs whose base sequences are shown in Table 23.

TABLE 23
Subset I

| Sense | Antisense |
|---|---|
| SEQ ID NO: | SEQ ID NO: |
| 888 | 6378 |
| 1683 | 7173 |
| 1684 | 7174 |
| 1912 | 7402 |
| 1922 | 7412 |
| 1927 | 7417 |
| 1938 | 7428 |
| 2014 | 7504 |
| 2167 | 7657 |
| 2213 | 7703 |
| 2362 | 7852 |
| 2364 | 7854 |
| 2365 | 7855 |
| 2598 | 8088 |
| 2631 | 8121 |
| 2759 | 8249 |
| 2890 | 8380 |
| 3231 | 8721 |
| 3259 | 8749 |
| 3333 | 8823 |
| 3487 | 8977 |
| 3491 | 8981 |
| 3586 | 9076 |
| 3587 | 9077 |
| 3756 | 9246 |

In some cases, position 9 of the sense strand can be a 2′deoxy. In these cases, 2′F and 2′OMe modifications may occur at the other positions of the sense strand.

In some cases, the sense strand of any of the siRNAs of subset I comprises a modification pattern which conforms to the sense strand rules as described for the sequences in Table 21 (Table 24).

In some cases, the antisense strand of any of the siRNAs of subset I comprise a modification or modification pattern. Some such examples are included in Table 24. Table 25 includes some additional sense strand modifications of the siRNAs in Table 23. The siRNAs in subset I may comprise any other modification pattern(s).

TABLE 24
Subset J

| SEQ | Sense strand sequence | SEQ | Antisense strand sequence |
|---|---|---|---|
| ID NO: | (5′-3′) | ID NO: | (5′-3′) |
| 11091 | acuacuAfuGfAfucuugucaasusu | 11116 | usUfsgAfcAfaGfaUfcAfuAfgUfaGfususu |
| 11092 | auggcuuAfAfuGfaucaguuasusu | 11117 | usAfsaCfuGfaUfcAfuUfaAfgCfcAfususu |
| 11093 | uggcUfUfaaUfgaucaguugasusu | 11118 | usCfsaAfcUfgAfuCfaUfuAfaGfcCfasusu |
| 11094 | uauuUfCfUfCfUfucauuaucgasusu | 11119 | usCfsgAfuAfaUfgAfaGfaGfaAfaUfasusu |
| 11095 | cauuaUfCfgUfaguacucuaasusu | 11120 | usUfsaGfaGfuAfcUfaCfgAfuAfaUfgsusu |
| 11096 | ucguagUfaCfUfcuagaucgasusu | 11121 | usCfsgAfuCfuAfgAfgUfaCfuAfcGfasusu |
| 11097 | uagaucGfaAfuGfuuagauuasusu | 11122 | usAfsaUfcUfaAfcAfuUfcGfaUfcUfasusu |
| 11098 | ucguUfUfugUfaguaaaagaasusu | 11123 | usUfscUfuUfuAfcUfaCfaAfaAfcGfasusu |
| 11099 | uuuuCfUfuaCfCfacauaucgasusu | 11124 | usCfsgAfuAfuGfuGfgUfaAfgAfaAfasusu |
| 11100 | agcuuAfcuGfAfuugaacggasusu | 11125 | usCfscGfuUfcAfaUfcAfgUfaAfgCfususu |
| 11101 | uguauuucGfGfcauuggguasusu | 11126 | usAfscCfcAfaUfgCfcGfaAfaUfaCfasusu |
| 11102 | uauuUfCfggCfaUfuggguugasusu | 11127 | usCfsaAfcCfcAfaUfgCfcGfaAfaUfasusu |
| 11103 | auuucGfGfcAfuuggguugaasusu | 11128 | usUfscAfaCfcCfaAfuGfcCfgAfaAfususu |
| 11104 | aguuuGfAfAfAfcAfuuugaucasusu | 11129 | usGfsaUfcAfaAfuGfuUfuCfaAfaCfususu |
| 11105 | caauAfGfAfAfAfuugcacgucasusu | 11130 | usGfsaCfgUfgCfaAfuUfuCfuAfuUfgsusu |
| 11106 | augaUfUfcgCfCfauaccacaasusu | 11131 | usUfsgUfgGfuAfuGfgCfgAfaUfcAfususu |
| 11107 | uuucAfAfuGfGfcGfuauuggaasusu | 11132 | usUfscCfaAfuAfcGfcCfaUfuGfaAfasusu |
| 11108 | uacgGfAfuAfGfaAfccagauaasusu | 11133 | usUfsaUfcUfgGfuUfcUfaUfcCfgUfasusu |
| 11109 | cuuuGfAfAfAfAfccuuaacccasusu | 11134 | usGfsgGfuUfaAfgGfuUfuUfcAfaAfgsusu |
| 11110 | aaauUfgaaCfCfucgaaacuasusu | 11135 | usAfsgUfuUfcGfaGfgUfuCfaAfuUfususu |
| 11111 | uaaaAfuAfaGfcuuuagucgasusu | 11136 | usCfsgAfcUfaAfaGfcUfuAfuUfuUfasusu |
| 11112 | auaagCfUfUfUfagucggauuasusu | 11137 | usAfsaUfcCfgAfcUfaAfaGfcUfuAfususu |
| 11113 | uucagaCfCfUfUfaguguauuasusu | 11138 | usAfsaUfaCfaCfuAfaGfgUfcUfgAfasusu |
| 11114 | ucagaCfCfUfUfaguguauuuasusu | 11139 | usAfsaAfuAfcAfcUfaAfgGfuCfuGfasusu |
| 11115 | aguuUfgaUfCfaUfgaugcuuasusu | 11140 | usAfsaGfcAfuCfaUfgAfuCfaAfaCfususu |

TABLE 25
Subset K

| SEQ | Sense strand sequence | SEQ ID | Antisense strand sequence |
|---|---|---|---|
| ID NO: | (5′-3′) | NO: | (5′-3′) |
| 11141 | uauuucuCfUfuCfauuaucgasusu | 11119 | usCfsgAfuAfaUfgAfaGfaGfaAfaUfasusu |
| 11142 | uauuucuCfUfUfCfauuaucgasusu | 11119 | usCfsgAfuAfaUfgAfaGfaGfaAfaUfasusu |
| 11143 | uauuucUfcUfuCfauuaucgasusu | 11119 | usCfsgAfuAfaUfgAfaGfaGfaAfaUfasusu |
| 11144 | uauuucUfcUfUfcauuaucgasusu | 11119 | usCfsgAfuAfaUfgAfaGfaGfaAfaUfasusu |
| 11145 | uauuucUfCfUfUfcauuaucgasusu | 11119 | usCfsgAfuAfaUfgAfaGfaGfaAfaUfasusu |
| 11146 | uauuucUfCfUfUfCfauuaucgasusu | 11119 | usCfsgAfuAfaUfgAfaGfaGfaAfaUfasusu |
| 11147 | uauuuCfucUfuCfauuaucgasusu | 11119 | usCfsgAfuAfaUfgAfaGfaGfaAfaUfasusu |
| 11148 | uauuuCfucUfUfcauuaucgasusu | 11119 | usCfsgAfuAfaUfgAfaGfaGfaAfaUfasusu |
| 11149 | uauuuCfuCfUfucauuaucgasusu | 11119 | usCfsgAfuAfaUfgAfaGfaGfaAfaUfasusu |
| 11150 | uauuuCfuCfUfuCfauuaucgasusu | 11119 | usCfsgAfuAfaUfgAfaGfaGfaAfaUfasusu |
| 11151 | uauuuCfuCfUfUfCfauuaucgasusu | 11119 | usCfsgAfuAfaUfgAfaGfaGfaAfaUfasusu |
| 11152 | uauuuCfUfcUfucauuaucgasusu | 11119 | usCfsgAfuAfaUfgAfaGfaGfaAfaUfasusu |
| 11153 | uauuuCfUfcUfuCfauuaucgasusu | 11119 | usCfsgAfuAfaUfgAfaGfaGfaAfaUfasusu |
| 11154 | uauuuCfUfcUfUfcauuaucgasusu | 11119 | usCfsgAfuAfaUfgAfaGfaGfaAfaUfasusu |
| 11155 | uauuuCfUfCfUfucauuaucgasusu | 11119 | usCfsgAfuAfaUfgAfaGfaGfaAfaUfasusu |
| 11156 | uauuuCfUfCfUfuCfauuaucgasusu | 11119 | usCfsgAfuAfaUfgAfaGfaGfaAfaUfasusu |
| 11157 | uauuuCfUfCfUfUfcauuaucgasusu | 11119 | usCfsgAfuAfaUfgAfaGfaGfaAfaUfasusu |
| 11158 | uauuUfcucUfuCfauuaucgasusu | 11119 | usCfsgAfuAfaUfgAfaGfaGfaAfaUfasusu |
| 11159 | uauuUfcucUfUfcauuaucgasusu | 11119 | usCfsgAfuAfaUfgAfaGfaGfaAfaUfasusu |
| 11160 | uauuUfcuCfUfucauuaucgasusu | 11119 | usCfsgAfuAfaUfgAfaGfaGfaAfaUfasusu |
| 11161 | uauuUfcuCfUfuCfauuaucgasusu | 11119 | usCfsgAfuAfaUfgAfaGfaGfaAfaUfasusu |
| 11162 | uauuUfcuCfUfUfCfauuaucgasusu | 11119 | usCfsgAfuAfaUfgAfaGfaGfaAfaUfasusu |
| 11163 | uauuUfcUfcUfucauuaucgasusu | 11119 | usCfsgAfuAfaUfgAfaGfaGfaAfaUfasusu |
| 11164 | uauuUfcUfcUfuCfauuaucgasusu | 11119 | usCfsgAfuAfaUfgAfaGfaGfaAfaUfasusu |
| 11165 | uauuUfcUfcUfUfcauuaucgasusu | 11119 | usCfsgAfuAfaUfgAfaGfaGfaAfaUfasusu |
| 11166 | uauuUfcUfCfUfUfcauuaucgasusu | 11119 | usCfsgAfuAfaUfgAfaGfaGfaAfaUfasusu |
| 11167 | uauuUfCfucUfucauuaucgasusu | 11119 | usCfsgAfuAfaUfgAfaGfaGfaAfaUfasusu |
| 11168 | uauuUfCfucUfuCfauuaucgasusu | 11119 | usCfsgAfuAfaUfgAfaGfaGfaAfaUfasusu |
| 11169 | uauuUfCfucUfUfcauuaucgasusu | 11119 | usCfsgAfuAfaUfgAfaGfaGfaAfaUfasusu |
| 11170 | uauuUfCfuCfUfucauuaucgasusu | 11119 | usCfsgAfuAfaUfgAfaGfaGfaAfaUfasusu |
| 11171 | uauuUfCfuCfUfuCfauuaucgasusu | 11119 | usCfsgAfuAfaUfgAfaGfaGfaAfaUfasusu |
| 11172 | ucguagUfaCfuCfuagaucgasusu | 11121 | usCfsgAfuCfuAfgAfgUfaCfuAfcGfasusu |
| 11173 | uagaucgAfAfuGfuuagauuasusu | 11122 | usAfsaUfcUfaAfcAfuUfcGfaUfcUfasusu |
| 11174 | ucguuUfUfgUfaguaaaagaasusu | 11123 | usUfscUfuUfuAfcUfaCfaAfaAfcGfasusu |
| 11175 | ucguUfuUfgUfaguaaaagaasusu | 11123 | usUfscUfuUfuAfcUfaCfaAfaAfcGfasusu |
| 11176 | uuuucuUfaCfCfacauaucgasusu | 11124 | usCfsgAfuAfuGfuGfgUfaAfgAfaAfasusu |
| 11177 | uuuucUfuaCfCfacauaucgasusu | 11124 | usCfsgAfuAfuGfuGfgUfaAfgAfaAfasusu |
| 11178 | uuuucUfUfaCfcacauaucgasusu | 11124 | usCfsgAfuAfuGfuGfgUfaAfgAfaAfasusu |
| 11179 | uuuucUfUfaCfCfacauaucgasusu | 11124 | usCfsgAfuAfuGfuGfgUfaAfgAfaAfasusu |
| 11180 | uuuuCfuuaCfCfacauaucgasusu | 11124 | usCfsgAfuAfuGfuGfgUfaAfgAfaAfasusu |
| 11181 | uuuuCfuUfaCfcacauaucgasusu | 11124 | usCfsgAfuAfuGfuGfgUfaAfgAfaAfasusu |
| 11182 | uuuuCfuUfaCfCfacauaucgasusu | 11124 | usCfsgAfuAfuGfuGfgUfaAfgAfaAfasusu |
| 11183 | uuuuCfUfuaCfcacauaucgasusu | 11124 | usCfsgAfuAfuGfuGfgUfaAfgAfaAfasusu |
| 11184 | uauuuCfggCfaUfuggguugasusu | 11127 | usCfsaAfcCfcAfaUfgCfcGfaAfaUfasusu |
| 11185 | uauuUfcggCfaUfuggguugasusu | 11127 | usCfsaAfcCfcAfaUfgCfcGfaAfaUfasusu |
| 11186 | uauuUfCfggCfauuggguugasusu | 11127 | usCfsaAfcCfcAfaUfgCfcGfaAfaUfasusu |
| 11187 | aguuugaAfAfcAfuuugaucasusu | 11129 | usGfsaUfcAfaAfuGfuUfuCfaAfaCfususu |
| 11188 | aguuugAfaAfcAfuuugaucasusu | 11129 | usGfsaUfcAfaAfuGfuUfuCfaAfaCfususu |
| 11189 | aguuuGfaaAfcAfuuugaucasusu | 11129 | usGfsaUfcAfaAfuGfuUfuCfaAfaCfususu |
| 11190 | aguuuGfaAfAfcauuugaucasusu | 11129 | usGfsaUfcAfaAfuGfuUfuCfaAfaCfususu |
| 11191 | aguuuGfaAfAfcAfuuugaucasusu | 11129 | usGfsaUfcAfaAfuGfuUfuCfaAfaCfususu |
| 11192 | aguuuGfAfaAfcauuugaucasusu | 11129 | usGfsaUfcAfaAfuGfuUfuCfaAfaCfususu |
| 11193 | aguuuGfAfaAfcAfuuugaucasusu | 11129 | usGfsaUfcAfaAfuGfuUfuCfaAfaCfususu |
| 11194 | aguuuGfAfAfAfcauuugaucasusu | 11129 | usGfsaUfcAfaAfuGfuUfuCfaAfaCfususu |
| 11195 | caauaGfaAfAfuugcacgucasusu | 11130 | usGfsaCfgUfgCfaAfuUfuCfuAfuUfgsusu |
| 11196 | caauaGfAfaAfuugcacgucasusu | 11130 | usGfsaCfgUfgCfaAfuUfuCfuAfuUfgsusu |
| 11197 | caauaGfAfAfAfuugcacgucasusu | 11130 | usGfsaCfgUfgCfaAfuUfuCfuAfuUfgsusu |
| 11198 | caauAfgaAfAfuugcacgucasusu | 11130 | usGfsaCfgUfgCfaAfuUfuCfuAfuUfgsusu |
| 11199 | caauAfgAfaAfuugcacgucasusu | 11130 | usGfsaCfgUfgCfaAfuUfuCfuAfuUfgsusu |
| 11200 | caauAfGfaaAfuugcacgucasusu | 11130 | usGfsaCfgUfgCfaAfuUfuCfuAfuUfgsusu |
| 11201 | caauAfGfaAfAfuugcacgucasusu | 11130 | usGfsaCfgUfgCfaAfuUfuCfuAfuUfgsusu |
| 11202 | augauuCfgCfCfauaccacaasusu | 11131 | usUfsgUfgGfuAfuGfgCfgAfaUfcAfususu |
| 11203 | augauUfcgCfCfauaccacaasusu | 11131 | usUfsgUfgGfuAfuGfgCfgAfaUfcAfususu |
| 11204 | augauUfCfgCfcauaccacaasusu | 11131 | usUfsgUfgGfuAfuGfgCfgAfaUfcAfususu |
| 11205 | augauUfCfgCfCfauaccacaasusu | 11131 | usUfsgUfgGfuAfuGfgCfgAfaUfcAfususu |
| 11206 | augaUfucgCfCfauaccacaasusu | 11131 | usUfsgUfgGfuAfuGfgCfgAfaUfcAfususu |
| 11207 | augaUfuCfgCfcauaccacaasusu | 11131 | usUfsgUfgGfuAfuGfgCfgAfaUfcAfususu |
| 11208 | augaUfuCfgCfCfauaccacaasusu | 11131 | usUfsgUfgGfuAfuGfgCfgAfaUfcAfususu |
| 11209 | augaUfUfcgCfcauaccacaasusu | 11131 | usUfsgUfgGfuAfuGfgCfgAfaUfcAfususu |
| 11210 | uuucaauGfGfcGfuauuggaasusu | 11132 | usUfscCfaAfuAfcGfcCfaUfuGfaAfasusu |
| 11211 | uuucaAfugGfcGfuauuggaasusu | 11132 | usUfscCfaAfuAfcGfcCfaUfuGfaAfasusu |
| 11212 | uuucaAfuGfGfcguauuggaasusu | 11132 | usUfscCfaAfuAfcGfcCfaUfuGfaAfasusu |
| 11213 | uuucaAfuGfGfcGfuauuggaasusu | 11132 | usUfscCfaAfuAfcGfcCfaUfuGfaAfasusu |
| 11214 | uuucAfaugGfcGfuauuggaasusu | 11132 | usUfscCfaAfuAfcGfcCfaUfuGfaAfasusu |
| 11215 | uuucAfauGfGfcguauuggaasusu | 11132 | usUfscCfaAfuAfcGfcCfaUfuGfaAfasusu |
| 11216 | uuucAfauGfGfcGfuauuggaasusu | 11132 | usUfscCfaAfuAfcGfcCfaUfuGfaAfasusu |
| 11217 | uuucAfAfugGfcguauuggaasusu | 11132 | usUfscCfaAfuAfcGfcCfaUfuGfaAfasusu |
| 11218 | uuucAfAfugGfcGfuauuggaasusu | 11132 | usUfscCfaAfuAfcGfcCfaUfuGfaAfasusu |
| 11219 | uuucAfAfuGfGfcguauuggaasusu | 11132 | usUfscCfaAfuAfcGfcCfaUfuGfaAfasusu |
| 11220 | uacggauAfGfaAfccagauaasusu | 11133 | usUfsaUfcUfgGfuUfcUfaUfcCfgUfasusu |
| 11221 | uacggauAfGfAfAfccagauaasusu | 11133 | usUfsaUfcUfgGfuUfcUfaUfcCfgUfasusu |
| 11222 | uacggAfuaGfaAfccagauaasusu | 11133 | usUfsaUfcUfgGfuUfcUfaUfcCfgUfasusu |
| 11223 | uacggAfuaGfAfaccagauaasusu | 11133 | usUfsaUfcUfgGfuUfcUfaUfcCfgUfasusu |
| 11224 | uacggAfuAfGfaaccagauaasusu | 11133 | usUfsaUfcUfgGfuUfcUfaUfcCfgUfasusu |
| 11225 | uacggAfuAfGfaAfccagauaasusu | 11133 | usUfsaUfcUfgGfuUfcUfaUfcCfgUfasusu |
| 11226 | uacggAfuAfGfAfAfccagauaasusu | 11133 | usUfsaUfcUfgGfuUfcUfaUfcCfgUfasusu |
| 11227 | uacgGfauaGfaAfccagauaasusu | 11133 | usUfsaUfcUfgGfuUfcUfaUfcCfgUfasusu |
| 11228 | uacgGfauaGfAfaccagauaasusu | 11133 | usUfsaUfcUfgGfuUfcUfaUfcCfgUfasusu |
| 11229 | uacgGfauAfGfaaccagauaasusu | 11133 | usUfsaUfcUfgGfuUfcUfaUfcCfgUfasusu |
| 11230 | uacgGfauAfGfaAfccagauaasusu | 11133 | usUfsaUfcUfgGfuUfcUfaUfcCfgUfasusu |
| 11231 | uacgGfauAfGfAfAfccagauaasusu | 11133 | usUfsaUfcUfgGfuUfcUfaUfcCfgUfasusu |
| 11232 | uacgGfAfuaGfaaccagauaasusu | 11133 | usUfsaUfcUfgGfuUfcUfaUfcCfgUfasusu |
| 11233 | uacgGfAfuaGfaAfccagauaasusu | 11133 | usUfsaUfcUfgGfuUfcUfaUfcCfgUfasusu |
| 11234 | uacgGfAfuaGfAfaccagauaasusu | 11133 | usUfsaUfcUfgGfuUfcUfaUfcCfgUfasusu |
| 11235 | uacgGfAfuAfGfaaccagauaasusu | 11133 | usUfsaUfcUfgGfuUfcUfaUfcCfgUfasusu |
| 11236 | cuuugAfaAfAfccuuaacccasusu | 11134 | usGfsgGfuUfaAfgGfuUfuUfcAfaAfgsusu |
| 11237 | cuuugAfAfaAfccuuaacccasusu | 11134 | usGfsgGfuUfaAfgGfuUfuUfcAfaAfgsusu |
| 11238 | cuuugAfAfAfAfccuuaacccasusu | 11134 | usGfsgGfuUfaAfgGfuUfuUfcAfaAfgsusu |
| 11239 | cuuuGfaaAfAfccuuaacccasusu | 11134 | usGfsgGfuUfaAfgGfuUfuUfcAfaAfgsusu |
| 11240 | cuuuGfaAfaAfccuuaacccasusu | 11134 | usGfsgGfuUfaAfgGfuUfuUfcAfaAfgsusu |
| 11241 | cuuuGfAfaaAfccuuaacccasusu | 11134 | usGfsgGfuUfaAfgGfuUfuUfcAfaAfgsusu |
| 11242 | cuuuGfAfaAfAfccuuaacccasusu | 11134 | usGfsgGfuUfaAfgGfuUfuUfcAfaAfgsusu |
| 11243 | aaauUfgaaCfcUfcgaaacuasusu | 11135 | usAfsgUfuUfcGfaGfgUfuCfaAfuUfususu |
| 11244 | uaaaAfuaAfGfcuuuagucgasusu | 11136 | usCfsgAfcUfaAfaGfcUfuAfuUfuUfasusu |
| 11245 | auaagCfuUfUfagucggauuasusu | 11137 | usAfsaUfcCfgAfcUfaAfaGfcUfuAfususu |
| 11246 | auaagCfUfuUfagucggauuasusu | 11137 | usAfsaUfcCfgAfcUfaAfaGfcUfuAfususu |
| 11247 | uucagaCfcUfUfaguguauuasusu | 11138 | usAfsaUfaCfaCfuAfaGfgUfcUfgAfasusu |
| 11248 | ucagaCfcUfUfaguguauuuasusu | 11139 | usAfsaAfuAfcAfcUfaAfgGfuCfuGfasusu |
| 11249 | ucagaCfCfuUfaguguauuuasusu | 11139 | usAfsaAfuAfcAfcUfaAfgGfuCfuGfasusu |
| 11250 | aguuugaUfCfaUfgaugcuuasusu | 11140 | usAfsaGfcAfuCfaUfgAfuCfaAfaCfususu |
| 11251 | aguuUfgauCfaUfgaugcuuasusu | 11140 | usAfsaGfcAfuCfaUfgAfuCfaAfaCfususu |
| 11252 | aguuUfgaUfCfaugaugcuuasusu | 11140 | usAfsaGfcAfuCfaUfgAfuCfaAfaCfususu |

### Example 11: Modification Motif 1

An example SOS2 siRNA includes a combination of the following modifications:
Position 9 (from 5′ to 3′) of the sense strand is 2′ F.
If position 9 is a pyrimidine then all purines in the Sense Strand are 2′OMe, and 1-5 pyrimidines between positions 5 and 11 are 2′ F provided that there are never three 2′F modifications in a row.
If position 9 is a purine then all pyrimidines in the Sense Strand are 2′OMe, and 1-5 purines between positions 5 and 11 are 2′ F provided that there are never three 2′F modifications in a row.
Antisense strand odd-numbered positions are 2′OMe and even-numbered positions are a mixture of 2′ F and 2′ deoxy.

### Example 12: Modification Motif 2

An example SOS2 siRNA includes a combination of the following modifications:
Position 9 (from 5′ to 3′) of the sense strand is 2′ deoxy.
Sense strand positions 5, 7 and 8 are 2′ F.
All pyrimidines in positions 10-21 are 2′ OMe, and purines are a mixture of 2′ OMe and 2′ F. Alternatively, all purines in positions 10-21 are 2′ OMe and all pyrimidines in positions 10-21 are a mixture of 2 OMe and 2 F.
Antisense strand odd-numbered positions are 2′OMe and even-numbered positions are a mixture of 2′ F and 2′ deoxy.

### Example 13: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence.

In solution phase conjugation, the oligonucleotide sequence—including a reactive conjugation site—is formed on the resin. The oligonucleotide is then removed from the resin and GalNAc is conjugated to the reactive site.

The carboxy GalNAc derivatives may be coupled to amino-modified oligonucleotides. The peptide coupling conditions are known to the skilled in the art using a carbodiimide coupling agent like DCC (N,N′-Dicyclohexylcarbodiimide), EDC (N-(3-dimethylaminopropyl)-N′-ethylcarbodiimide) or EDC·HCl (N-(3-dimethylaminopropyl)-N′-ethylcarbodiimide hydrochloride and an additive like HOBt (1-hydroxybenztriazole), HOSu (N-hydroxysuccinimide), TBTU (N,N,N′,N′-Tetramethyl-O-(benzotriazol-1-yl)uronium tetrafluoroborate, HBTU (2-(1H-benzotriazol-1-yl)-1,1,3,3-tetramethyluronium hexafluorophosphate) or HOAt (1-Hydroxy-7-azabenzotriazole and common combinations thereof such as TBTU/HOBt or HBTU/HOAt to form activated amine-reactive esters.

Amine groups may be incorporated into oligonucleotides using a number of known, commercially available reagents at the 5′ terminus, 3′ terminus or anywhere in between.

Non-limiting examples of reagents for oligonucleotide synthesis to incorporate an amino group include:
5′ attachment:
   6-(4-Monomethoxytritylamino)hexyl-(2-cyanoethyl)-(N,N-diisopropyl)-phosphoramidite CAS Number: 114616-27-2
   5′-Amino-Modifier TEG CE-Phosphoramidite
   10-(O-trifluoroacetamido-N-ethyl)-triethyleneglycol-1-[(2-cyanoethyl)-(N,N-diisopropyl)]-phosphoramidite
3′ attachment:
   3′-Amino-Modifier Serinol CPG
   3-Dimethoxytrityloxy-2-(3-(fluorenylmethoxycarbonylamino)propanamido)propyl-1-O-succinyl-long chain alkylamino-CPG (where CPG stands for controlled-pore glass and is the solid support)
   Amino-Modifier Serinol Phosphoramidite
   3-Dimethoxytrityloxy-2-(3-(fluorenylmethoxycarbonylamino)propanamido)propyl-1-O-(2-cyanoethyl)-(N,N-diisopropyl)-phosphoramidite
Internal (base modified):
   Amino-Modifier C6 dT
   5′-Dimethoxytrityl-5-[N-(trifluoroacetylaminohexyl)-3-acrylimido]-2′-deoxyUridine,3′-[(2-cyanoethyl)-(N,N-diisopropyl)]-phosphoramidite. CAS Number: 178925-21-8

Solution phase conjugations may occur after oligonucleotide synthesis via reactions between non-nucleosidic nucleophilic functional groups that are attached to the oligonucleotide and electrophilic GalNAc reagents. Examples of nucleophilic groups include amines and thiols, and examples of electrophilic reagents include activated esters (e.g. N-hydroxysuccinimide, pentafluorophenyl) and maleimides.

TABLE 26
GalNAc Conjugation Reagent

| Type of | |
|---|---|
| conjugation | Structure |
| Solid phase 5' attachment phosphoramidite | 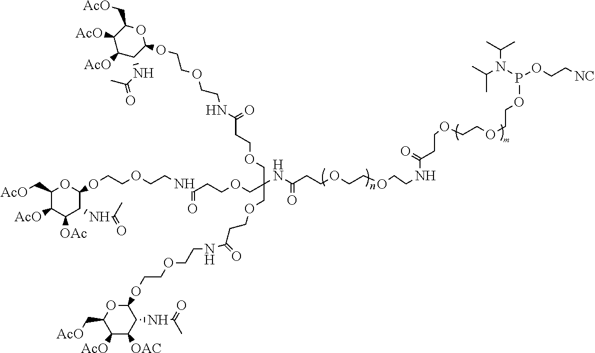 |

An example of a reagent for GalNAc conjugation to an oligonucleotide is shown in Table 26. For the phosphoramidite in Table 26, m or n may be any value from 1 to 10. For example, m may be 4, and n may be 2. The following is a non-limiting example of a scheme for synthesizing the phosphoramidite in Table 26.

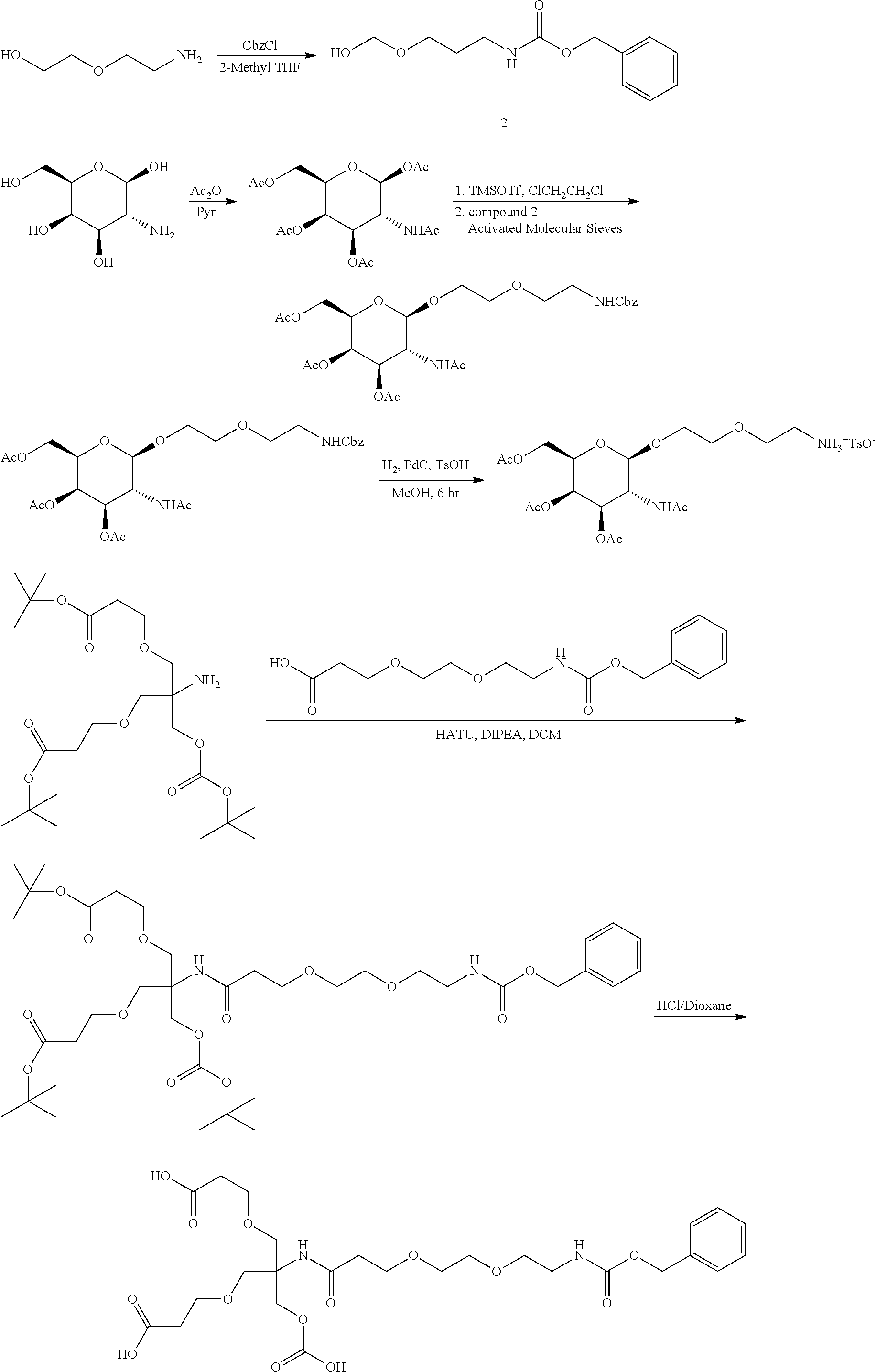

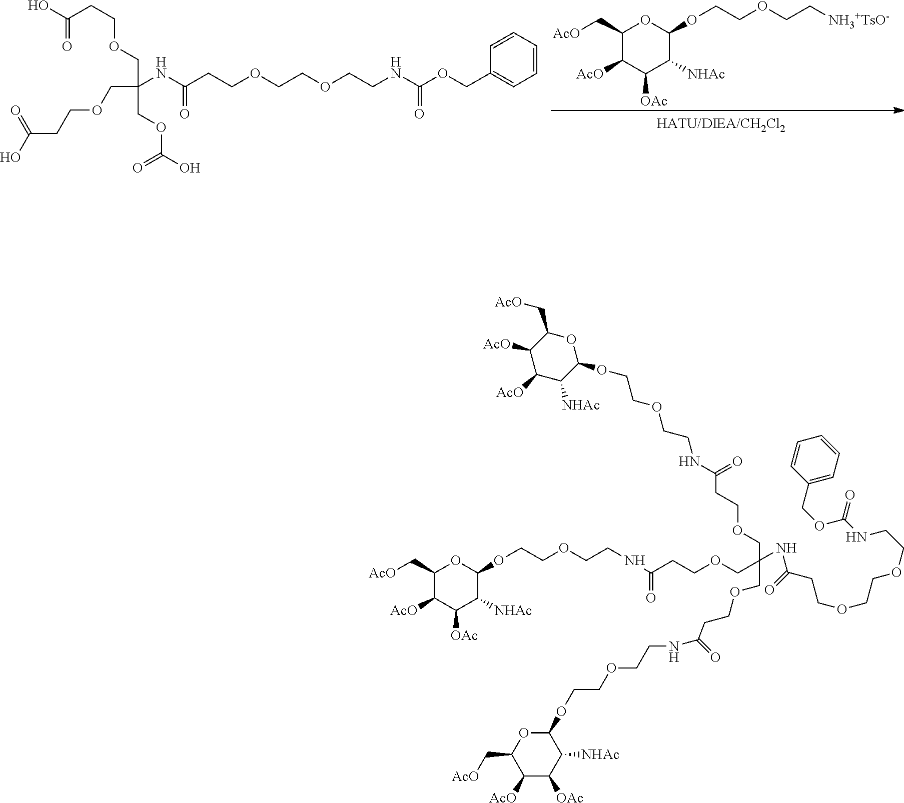

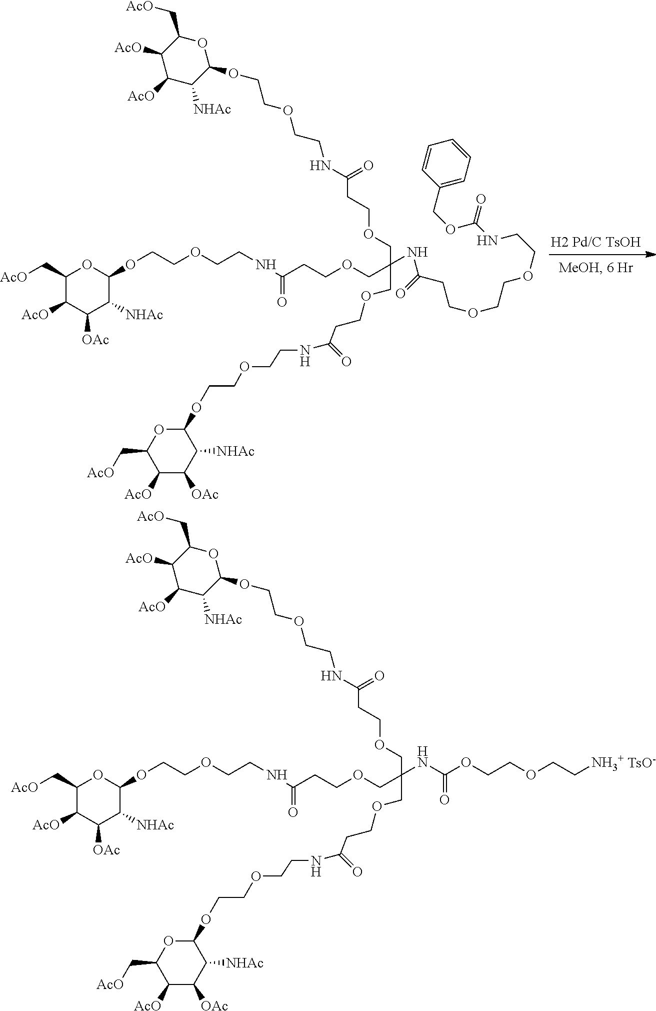

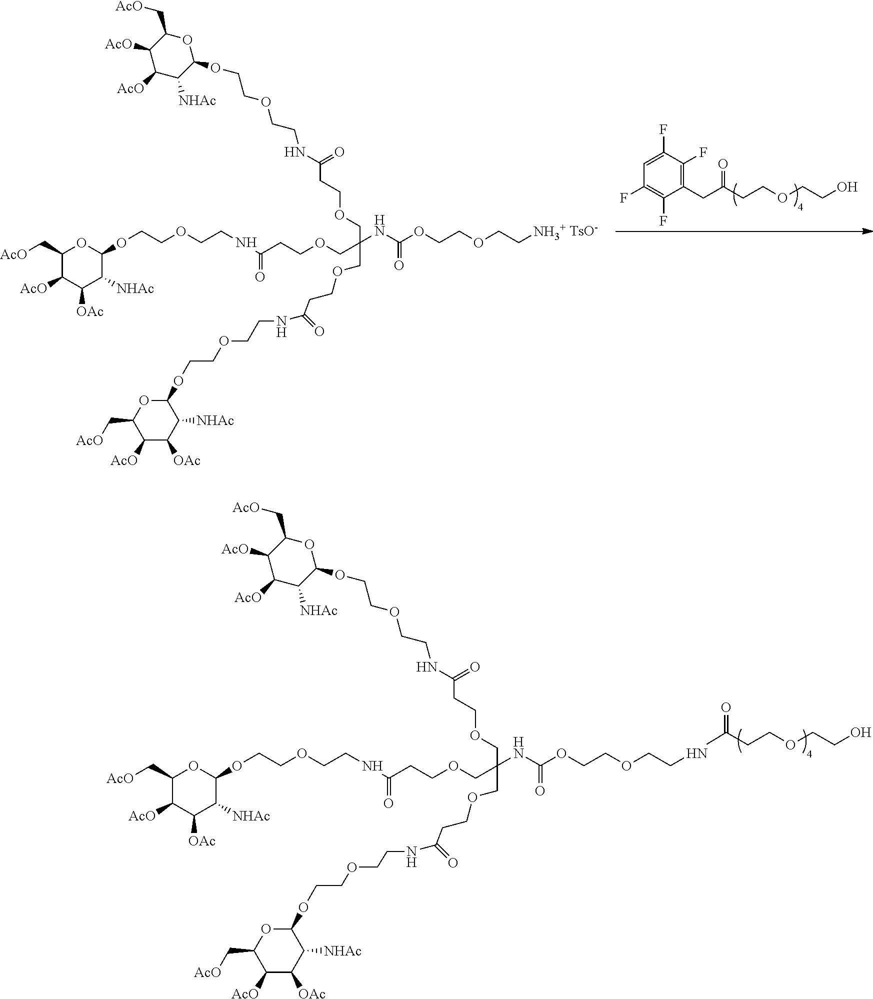

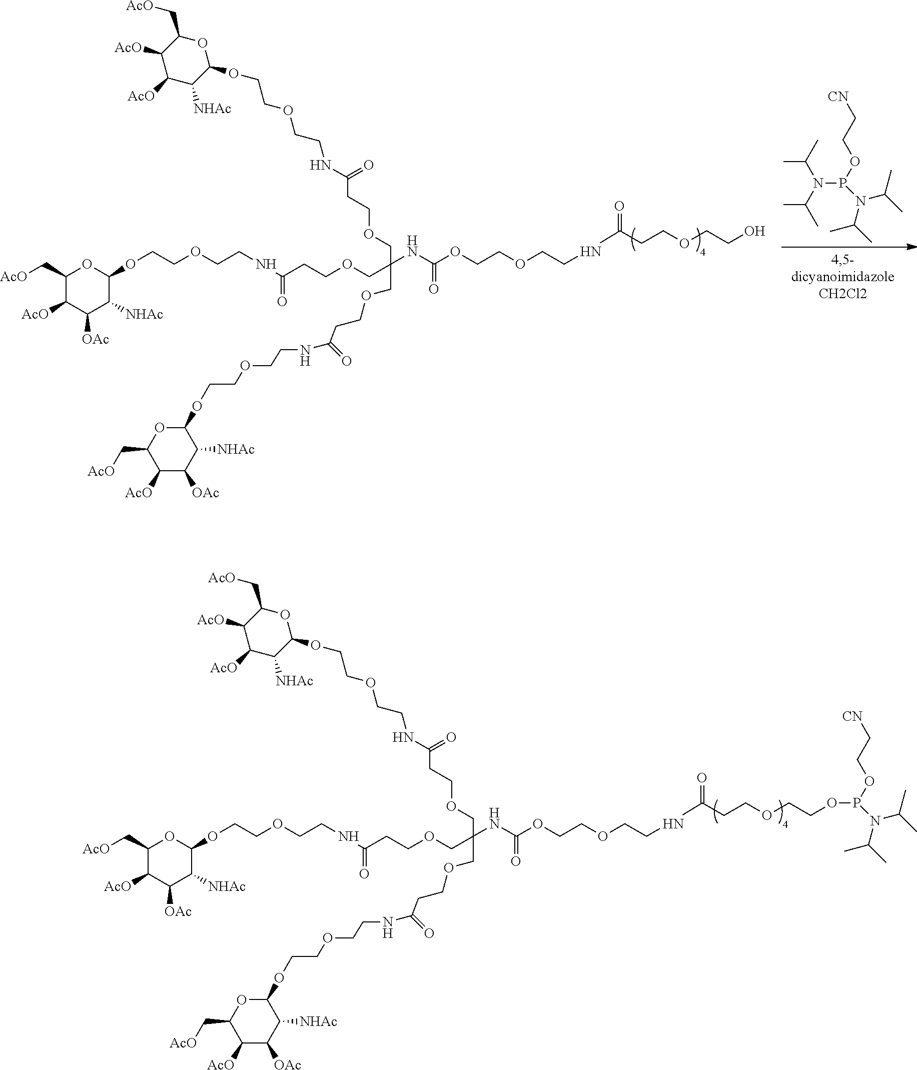

### Example 14: Screening of siRNAs Targeting Human SOS2 mRNA in Mice Transfected with AAV8-TBG-h-SOS2(5UTR+ORF-Nter)

Several siRNAs designed to be cross-reactive with human, cynomolgus monkey, rat and mouse SOS2 mRNA were tested for activity in mice following transfection with an adeno-associated viral vector. The siRNAs were attached to the GalNAc ligand ETL17. The siRNA sequences that were used are shown in Table 27, where Nf (e.g. Af, Cf, Gf, Tf, or Uf) is a 2′ fluoro-modified nucleoside, n (e.g. a, c, g, t, or u) is a 2′ O-methyl modified nucleoside, dN (e.g. dA, dC, dG, dT, or dU) is a deoxynucleoside, and “s” is a phosphorothioate linkage.

Six to eight week old female mice (C57Bl/6) were injected with 10 uL of a recombinant adeno-associated virus 8 (AAV8) vector (1.2×10E13 genome copies/mL) plus 20 ul of PBS for a total volume of 30 ul by the retroorbital route on Day −12. The recombinant AAV8 contains a portion of the 5′ UTR and a portion of the open reading frame of the human SOS2 sequence (ENST00000216373) under the control of the human thyroxine binding globulin promoter in an AAV2 backbone packaged in AAV8 capsid (AAV8-TBG-h-SOS2(5UTR+ORF-Nter)). On Day 0, infected mice (n=5) were given a subcutaneous injection of a single 100 ug dose of a GalNAc-conjugated siRNA or PBS as vehicle control.

Mice were euthanized on Day 10 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver SOS2 mRNA were assessed by RT-qPCR in triplicate on a QuantStudio™ 6 Pro Real-Time PCR System using TaqMan assays for human SOS2 (ThermoFisher, assay #Hs01127288_m1), or mouse SOS2 (ThermoFisher, assay #Mm01265231_m1) and the mouse housekeeping gene PPIA (ThermoFisher, assay #Mm02342430_g1) and PerfeCTa® qPCR FastMix®, Low ROX™ (VWR, Catalog #101419-222). Data were normalized to the mean SOS2 mRNA level in animals receiving PBS. Mice injected with ETD02117, ETD02118, ETD02121, ETD02122, ETD02123 and ETD02124 had substantial reductions in mean liver mouse SOS2 mRNA on Day 10 relative to mice receiving PBS. Results are shown in Table 29. Mice injected with ETD02117, ETD02118, ETD2121, ETD02122, ETD02123 and ETD02124 had substantial reductions in mean liver human SOS2 mRNA on Day 10 relative to mice receiving PBS. Results are shown in Table 30.

TABLE 27
Example siRNA Sequences

| | SS | | AS | |
|---|---|---|---|---|
| | SEQ | | SEQ | |
| siRNA | ID | Sense Strand (SS) Sequence | ID | Antisense Strand (AS) Sequence |
| Name | NO: | (5′-3′) with GalNAc moiety | NO: | (5′-3′) |
| ETD02115 | 11254 | [ETL17]suuuaaUfaUfCfcggcauuauasusu | 11264 | usAfsuAfaUfgCfcGfgAfuAfuUfaAfasusu |
| ETD02116 | 11255 | [ETL17]suuaaUfaUfcCfggcauuaugasusu | 11265 | usCfsaUfaAfuGfcCfgGfaUfaUfuAfasusu |
| ETD02117 | 11256 | [ETL17]suucagaUfaUfaCfaugaauuasusu | 11266 | usAfsaUfuCfaUfgUfaUfaUfcUfgAfasusu |
| ETD02118 | 11257 | [ETL17]scuguGfaAfAfdCUfuuuagguuasusu | 11267 | usAfsaCfcUfaAfaAfgUfuUfcAfcAfgsusu |
| ETD02119 | 11258 | [ETL17]sacauuAfucAfcAfggacauuasusu | 11268 | usAfsaUfgUfcCfuGfuGfaUfaAfuGfususu |
| ETD02120 | 11259 | [ETL17]saauuAfAfGfAfAfgcaaacaccasusu | 11269 | usGfsgUfgUfuUfgCfuUfcUfuAfaUfususu |
| ETD02121 | 11260 | [ETL17]sauccCfaaUfUfUfUfguucguaasusu | 11270 | usUfsaCfgAfaCfaAfaAfuUfgGfgAfususu |
| ETD02122 | 11261 | [ETL17]sccaaUfuUfUfdGUfucguacuuasusu | 11271 | usAfsaGfuAfcGfaAfcAfaAfaUfuGfgsusu |
| ETD02123 | 11262 | [ETL17]scaauUfUfugUfUfcguacuuuasusu | 11272 | usAfsaAfgUfaCfgAfaCfaAfaAfuUfgsusu |
| ETD02124 | 11263 | [ETL17]suguuCfgUfaCfUfuuucuuacasusu | 11273 | usGfsuAfaGfaAfaAfgUfaCfgAfaCfasusu |

TABLE 28
Example siRNA Base Sequences

| | SS | | AS | |
|---|---|---|---|---|
| | SEQ | | SEQ | |
| siRNA | ID | Sense Strand (SS) Base Sequence | ID | Antisense Strand (AS) Base |
| Name | NO: | (5′ to 3′) | NO: | Sequence (5′ to 3′) |
| ETD02115 | 11274 | UUUAAUAUCCGGCAUUAUAUU | 11304 | UAUAAUGCCGGAUAUUAAAUU |
| ETD02116 | 11275 | UUAAUAUCCGGCAUUAUGAUU | 11305 | UCAUAAUGCCGGAUAUUAAUU |
| ETD02117 | 11276 | UUCAGAUAUACAUGAAUUAUU | 11306 | UAAUUCAUGUAUAUCUGAAUU |
| ETD02118 | 11277 | CUGUGAAACUUUUAGGUUAUU | 11307 | UAACCUAAAAGUUUCACAGUU |
| ETD02119 | 11278 | ACAUUAUCACAGGACAUUAUU | 11308 | UAAUGUCCUGUGAUAAUGUUU |
| ETD02120 | 11279 | AAUUAAGAAGCAAACACCAUU | 11309 | UGGUGUUUGCUUCUUAAUUUU |
| ETD02121 | 11280 | AUCCCAAUUUUGUUCGUAAUU | 11310 | UUACGAACAAAAUUGGGAUUU |
| ETD02122 | 11281 | CCAAUUUUGUUCGUACUUAUU | 11311 | UAAGUACGAACAAAAUUGGUU |
| ETD02123 | 11282 | CAAUUUUGUUCGUACUUUAUU | 11312 | UAAAGUACGAACAAAAUUGUU |
| ETD02124 | 11283 | UGUUCGUACUUUUCUUACAUU | 11313 | UGUAAGAAAAGUACGAACAUU |
| | SS | | AS | |
| | SEQ | | SEQ | |
| SIRNA | ID | Sense Strand Base Sequence | ID | Antisense Strand Base Sequence |
| Name | NO: | (5′ to 3′), without 3′ overhang | NO: | (5′ to 3′), without 3′ overhang |
| ETD02115 | 11284 | UUUAAUAUCCGGCAUUAUA | 11314 | UAUAAUGCCGGAUAUUAAA |
| ETD02116 | 11285 | UUAAUAUCCGGCAUUAUGA | 11315 | UCAUAAUGCCGGAUAUUAA |
| ETD02117 | 11286 | UUCAGAUAUACAUGAAUUA | 11316 | UAAUUCAUGUAUAUCUGAA |
| ETD02118 | 11287 | CUGUGAAACUUUUAGGUUA | 11317 | UAACCUAAAAGUUUCACAG |
| ETD02119 | 11288 | ACAUUAUCACAGGACAUUA | 11318 | UAAUGUCCUGUGAUAAUGU |
| ETD02120 | 11289 | AAUUAAGAAGCAAACACCA | 11319 | UGGUGUUUGCUUCUUAAUU |
| ETD02121 | 11290 | AUCCCAAUUUUGUUCGUAA | 11320 | UUACGAACAAAAUUGGGAU |
| ETD02122 | 11291 | CCAAUUUUGUUCGUACUUA | 11321 | UAAGUACGAACAAAAUUGG |
| ETD02123 | 11292 | CAAUUUUGUUCGUACUUUA | 11322 | UAAAGUACGAACAAAAUUG |
| ETD02124 | 11293 | UGUUCGUACUUUUCUUACA | 11323 | UGUAAGAAAAGUACGAACA |
| | SS | | AS | |
| | SEQ | Sense Strand Base Sequence | SEQ | Antisense Strand Base Sequence |
| siRNA | ID | (5′ to 3′), | ID | (5′ to 3′), without 3′ |
| Name | NO: | without 3′ overhang and 3′ A | NO: | overhang and 5′U |
| ETD02115 | 11294 | UUUAAUAUCCGGCAUUAU | 11324 | AUAAUGCCGGAUAUUAAA |
| ETD02116 | 11295 | UUAAUAUCCGGCAUUAUG | 11325 | CAUAAUGCCGGAUAUUAA |
| ETD02117 | 11296 | UUCAGAUAUACAUGAAUU | 11326 | AAUUCAUGUAUAUCUGAA |
| ETD02118 | 11297 | CUGUGAAACUUUUAGGUU | 11327 | AACCUAAAAGUUUCACAG |
| ETD02119 | 11298 | ACAUUAUCACAGGACAUU | 11328 | AAUGUCCUGUGAUAAUGU |
| ETD02120 | 11299 | AAUUAAGAAGCAAACACC | 11329 | GGUGUUUGCUUCUUAAUU |
| ETD02121 | 11300 | AUCCCAAUUUUGUUCGUA | 11330 | UACGAACAAAAUUGGGAU |
| ETD02122 | 11301 | CCAAUUUUGUUCGUACUU | 11331 | AAGUACGAACAAAAUUGG |
| ETD02123 | 11302 | CAAUUUUGUUCGUACUUU | 11332 | AAAGUACGAACAAAAUUG |
| ETD02124 | 11303 | UGUUCGUACUUUUCUUAC | 11333 | GUAAGAAAAGUACGAACA |

TABLE 29
Relative mouse SOS2 mRNA Levels in Livers of Mice

| | | | | Mean mouse SOS2 mRNA |
|---|---|---|---|---|
| | | | Dose | (Normalized to Group 1, |
| Group | n | Treatment | (ug) | Day 10) |
| 1 | 5 | Control | 0 | 1.00 |
| 2 | 5 | Control + AAV8 | 0 | 1.05 |
| 3 | 5 | ETD02115 | 100 | 0.89 |
| 4 | 5 | ETD02116 | 100 | 0.72 |
| 5 | 5 | ETD02117 | 100 | 0.49 |
| 6 | 5 | ETD02118 | 100 | 0.31 |
| 7 | 5 | ETD02119 | 100 | 0.77 |
| 8 | 5 | ETD02120 | 100 | 0.80 |
| 9 | 5 | ETD02121 | 100 | 0.46 |
| 10 | 5 | ETD02122 | 100 | 0.49 |
| 11 | 5 | ETD02123 | 100 | 0.39 |
| 12 | 5 | ETD02124 | 100 | 0.20 |

TABLE 30
Relative human SOS2 mRNA Levels in Livers of Mice

| | | | | Mean human SOS2 mRNA |
|---|---|---|---|---|
| | | | Dose | (Normalized to Group 2, |
| Group | n | Treatment | (ug) | Day 10) |
| 1 | 5 | Control | 0 | N/A |
| 2 | 5 | Control + AAV8 | 0 | 1.00 |
| 3 | 5 | ETD02115 | 100 | 0.68 |
| 4 | 5 | ETD02116 | 100 | 0.46 |
| 5 | 5 | ETD02117 | 100 | 0.32 |
| 6 | 5 | ETD02118 | 100 | 0.25 |
| 7 | 5 | ETD02119 | 100 | 0.47 |
| 8 | 5 | ETD02120 | 100 | 0.23 |
| 9 | 5 | ETD02121 | 100 | 0.15 |
| 10 | 5 | ETD02122 | 100 | 0.15 |
| 11 | 5 | ETD02123 | 100 | 0.43 |
| 12 | 5 | ETD02124 | 100 | 0.23 |

### Example 15. Screening of siRNAs Targeting Human SOS2 mRNA in Mice Transfected with AAV8-TBG-h-SOS2(ORF-Cter+3′UTR)

Several siRNAs designed to be cross-reactive with human, cynomolgus monkey, rat and mouse SOS2 mRNA were tested for activity in mice following transfection with an adeno-associated viral vector. The siRNAs were attached to the GalNAc ligand ETL17. The siRNA sequences are shown in Table 31, where Nf (e.g. Af, Cf, Gf, Tf, or Uf) is a 2′ fluoro-modified nucleoside, n (e.g. a, c, g, t, or u) is a 2′ O-methyl modified nucleoside, dN (e.g. dA, dC, dG, dT, or dU) is a deoxynucleoside, and “s” is a phosphorothioate linkage.

Six to eight week old female mice (C57Bl/6) were injected with 10 uL of a recombinant adeno-associated virus 8 (AAV8) vector (1.34×10E13 genome copies/mL) plus 20 ul of PBS for a total volume of 30 ul by the retroorbital route on Day −12. The recombinant AAV8 contains a portion of the open reading frame and a portion of the 3′ UTR of the human SOS2 sequence (ENST00000216373) under the control of the human thyroxine binding globulin promoter in an AAV2 backbone packaged in AAV8 capsid (AAV8-TBG-h-SOS2(ORF-Cter+3′UTR)). On Day 0, infected mice (n=5) were given a subcutaneous injection of a single 100 ug dose of a GalNAc-conjugated siRNA or PBS as vehicle control.

Mice were euthanized on Day 10 after subcutaneous injection and a liver sample from each was collected and placed in RNAlater (ThermoFisher Catalog #AM7020) until processing. Total liver RNA was prepared by homogenizing the liver tissue in homogenization buffer (Maxwell RSC simplyRNA Tissue Kit) using a Percellys 24 tissue homogenizer (Bertin Instruments) set at 5000 rpm for two 10 second cycles. Total RNA from the lysate was purified on a Maxwell RSC 48 platform (Promega Corporation) according to the manufacturer's recommendations. Preparation of cDNA was performed using Quanta qScript cDNA SuperMix (VWR, Catalog #95048-500) according to the manufacturer's instructions. The relative levels of liver SOS2 mRNA were assessed by RT-qPCR in triplicate on a QuantStudio™ 6 Pro Real-Time PCR System using TaqMan assays for human SOS2 (ThermoFisher, assay #Hs01127273_m1), and the mouse housekeeping gene PPIA (ThermoFisher, assay #Mm02342430_g1) and PerfeCTa® qPCR FastMix®, Low ROX™ (VWR, Catalog #101419-222). Mice injected with ETD02125, ETD02126, ETD02127, ETD02128, ETD02129, and ETD02133 had substantial reductions in mean liver human SOS2 mRNA on Day 10 relative to mice receiving PBS. Results are shown in Table 33.

TABLE 31
Example siRNA Sequences

| | SS | | AS | |
|---|---|---|---|---|
| | SEQ | | SEQ | |
| siRNA | ID | Sense Strand Sequence (5′-3′) | ID | |
| Name | NO: | with GalNAc moiety | NO: | Antisense Strand Sequence (5′-3′) |
| ETD02125 | 11334 | [ETL17]sugacaCfUfUfCfaUfccaauagasusu | 11344 | usCfsuAfuUfgGfaUfgAfaGfuGfuCfasusu |
| ETD02126 | 11335 | [ETL17]suugaaUfaaUfUfucaauggcasusu | 11345 | usGfscCfaUfuGfaAfaUfuAfuUfcAfasusu |
| ETD02127 | 11336 | [ETL17]sugaaUfaaUfUfUfCfaauggcgasusu | 11346 | usCfsgCfcAfuUfgAfaAfuUfaUfuCfasusu |
| ETD02128 | 11337 | [ETL17]sgaauaaUfUfUfCfaauggcguasusu | 11347 | usAfscGfcCfaUfuGfaAfaUfuAfuUfcsusu |
| ETD02129 | 11338 | [ETL17]saacuUfaagUfCfaaucaaucasusu | 11348 | usGfsaUfuGfaUfuGfaCfuUfaAfgUfususu |
| ETD02130 | 11339 | [ETL17]sacagaUfUfaUfUfuguucaacasusu | 11349 | usGfsuUfgAfaCfaAfaUfaAfuCfuGfususu |
| ETD02131 | 11340 | [ETL17]saacaCfuUfUfdAuAfaacugucasusu | 11350 | usGfsaCfaGfuUfuAfuAfaAfgUfgUfususu |
| ETD02132 | 11341 | [ETL17]saaagAfcuuAfcAfaacgggaasusu | 11351 | usUfscCfcGfuUfuGfuAfaGfuCfuUfususu |
| ETD02133 | 11342 | [ETL17]sauguAfGfucAfuugacacugasusu | 11352 | usCfsaGfuGfuCfaAfuGfaCfuAfcAfususu |
| ETD02134 | 11343 | [ETL17]sagucAfuuGfAfcAfcuggaauasusu | 11353 | usAfsuUfcCfaGfuGfuCfaAfuGfaCfususu |

TABLE 32
Example siRNA Base Sequences

| | SS | | AS | |
|---|---|---|---|---|
| | SEQ | | SEQ | |
| siRNA | ID | Sense Strand Base Sequence | ID | Antisense Strand Base Sequence |
| Name | NO: | (5′ to 3′) | NO: | (5′ to 3′) |
| ETD02125 | 11354 | UGACACUUCAUCCAAUAGAUU | 11384 | UCUAUUGGAUGAAGUGUCAUU |
| ETD02126 | 11355 | UUGAAUAAUUUCAAUGGCAUU | 11385 | UGCCAUUGAAAUUAUUCAAUU |
| ETD02127 | 11356 | UGAAUAAUUUCAAUGGCGAUU | 11386 | UCGCCAUUGAAAUUAUUCAUU |
| ETD02128 | 11357 | GAAUAAUUUCAAUGGCGUAUU | 11387 | UACGCCAUUGAAAUUAUUCUU |
| ETD02129 | 11358 | AACUUAAGUCAAUCAAUCAUU | 11388 | UGAUUGAUUGACUUAAGUUUU |
| ETD02130 | 11359 | ACAGAUUAUUUGUUCAACAUU | 11389 | UGUUGAACAAAUAAUCUGUUU |
| ETD02131 | 11360 | AACACUUUAUAAACUGUCAUU | 11390 | UGACAGUUUAUAAAGUGUUUU |
| ETD02132 | 11361 | AAAGACUUACAAACGGGAAUU | 11391 | UUCCCGUUUGUAAGUCUUUUU |
| ETD02133 | 11362 | AUGUAGUCAUUGACACUGAUU | 11392 | UCAGUGUCAAUGACUACAUUU |
| ETD02134 | 11363 | AGUCAUUGACACUGGAAUAUU | 11393 | UAUUCCAGUGUCAAUGACUUU |
| | SS | | AS | |
| | SEQ | | SEQ | |
| siRNA | ID | Sense Strand Base Sequence | ID | Antisense Strand Base Sequence |
| Name | NO: | (5′ to 3′), without 3′ overhang | NO: | (5′ to 3′), without 3′ overhang |
| ETD02125 | 11364 | UGACACUUCAUCCAAUAGA | 11394 | UCUAUUGGAUGAAGUGUCA |
| ETD02126 | 11365 | UUGAAUAAUUUCAAUGGCA | 11395 | UGCCAUUGAAAUUAUUCAA |
| ETD02127 | 11366 | UGAAUAAUUUCAAUGGCGA | 11396 | UCGCCAUUGAAAUUAUUCA |
| ETD02128 | 11367 | GAAUAAUUUCAAUGGCGUA | 11397 | UACGCCAUUGAAAUUAUUC |
| ETD02129 | 11368 | AACUUAAGUCAAUCAAUCA | 11398 | UGAUUGAUUGACUUAAGUU |
| ETD02130 | 11369 | ACAGAUUAUUUGUUCAACA | 11399 | UGUUGAACAAAUAAUCUGU |
| ETD02131 | 11370 | AACACUUUAUAAACUGUCA | 11400 | UGACAGUUUAUAAAGUGUU |
| ETD02132 | 11371 | AAAGACUUACAAACGGGAA | 11401 | UUCCCGUUUGUAAGUCUUU |
| ETD02133 | 11372 | AUGUAGUCAUUGACACUGA | 11402 | UCAGUGUCAAUGACUACAU |
| ETD02134 | 11373 | AGUCAUUGACACUGGAAUA | 11403 | UAUUCCAGUGUCAAUGACU |
| | SS | | AS | |
| | SEQ | Sense Strand Base Sequence | SEQ | Antisense Strand Base Sequence |
| siRNA | ID | (5′ to 3′), | ID | (5′ to 3′), without 3′ |
| Name | NO: | without 3′ overhang and 3′ A | NO: | overhang and 5′U |
| ETD02125 | 11374 | UGACACUUCAUCCAAUAG | 11404 | CUAUUGGAUGAAGUGUCA |
| ETD02126 | 11375 | UUGAAUAAUUUCAAUGGC | 11405 | GCCAUUGAAAUUAUUCAA |
| ETD02127 | 11376 | UGAAUAAUUUCAAUGGCG | 11406 | CGCCAUUGAAAUUAUUCA |
| ETD02128 | 11377 | GAAUAAUUUCAAUGGCGU | 11407 | ACGCCAUUGAAAUUAUUC |
| ETD02129 | 11378 | AACUUAAGUCAAUCAAUC | 11408 | GAUUGAUUGACUUAAGUU |
| ETD02130 | 11379 | ACAGAUUAUUUGUUCAAC | 11409 | GUUGAACAAAUAAUCUGU |
| ETD02131 | 11380 | AACACUUUAUAAACUGUC | 11410 | GACAGUUUAUAAAGUGUU |
| ETD02132 | 11381 | AAAGACUUACAAACGGGA | 11411 | UCCCGUUUGUAAGUCUUU |
| ETD02133 | 11382 | AUGUAGUCAUUGACACUG | 11412 | CAGUGUCAAUGACUACAU |
| ETD02134 | 11383 | AGUCAUUGACACUGGAAU | 11413 | AUUCCAGUGUCAAUGACU |

TABLE 33
Relative human SOS2 mRNA Levels in Livers of Mice

| | | | | Mean human SOS2 mRNA |
|---|---|---|---|---|
| | | | Dose | (Normalized to Group 2, |
| Group | n | Treatment | (ug) | Day 10) |
| 1 | 5 | Control | 0 | N/A |
| 2 | 5 | Control + AAV8 | 0 | 1.00 |
| 3 | 5 | ETD02125 | 100 | 0.30 |
| 4 | 5 | ETD02126 | 100 | 0.39 |
| 5 | 5 | ETD02127 | 100 | 0.36 |
| 6 | 5 | ETD02128 | 100 | 0.29 |
| 7 | 5 | ETD02129 | 100 | 0.41 |
| 8 | 5 | ETD02130 | 100 | 0.82 |
| 9 | 5 | ETD02131 | 100 | 0.76 |
| 10 | 5 | ETD02132 | 100 | 0.76 |
| 11 | 5 | ETD02133 | 100 | 0.54 |
| 12 | 5 | ETD02134 | 100 | 0.76 |

### IV. SEQUENCE INFORMATION

Some embodiments include one or more nucleic
acid sequences in the following tables:

| SEQ ID NO: | Description of Examples |
|---|---|
| 1-5490 | Sense strand sequences of SOS2 siRNAs |
| 5491-10980 | Antisense strand sequences of SOS2 siRNAs |
| 10981-11000 | Modified sense strand sequences of SOS2 siRNAs |
| 11001-11020 | Modified antisense strand sequences of SOS2 siRNAs |
| 11021-11090 | Modified sense strand sequences of SOS2 siRNAs |
| 11091-1115 | Modified sense strand sequences of SOS2 siRNAs |
| 11116-11140 | Modified antisense strand sequences of SOS2 siRNAs |
| 11141-11252 | Modified sense strand sequences of SOS2 siRNAs |
| 11253 | Full-length human SOS2 mRNA sequence (Ensembl |
| | Acc.# ENST00000216373.10) |
| 11254-11413 | Example sequences of SOS2 siRNAs |

| | SEQ | | | |
|---|---|---|---|---|
| | ID | sense strand sequence | SEQ ID | antisense strand sequence |
| Name | NO: | (5′-3′) | NO: | (5′-3′) |
| siRNA 1 | 1 | AGAAGCGGGCCAGCGCCGC | 5491 | GCGGCGCUGGCCCGCUUCU |
| siRNA 2 | 2 | GAAGCGGGCCAGCGCCGCC | 5492 | GGCGGCGCUGGCCCGCUUC |
| siRNA 3 | 3 | AAGCGGGCCAGCGCCGCCG | 5493 | CGGCGGCGCUGGCCCGCUU |
| siRNA 4 | 4 | AGCGGGCCAGCGCCGCCGG | 5494 | CCGGCGGCGCUGGCCCGCU |
| siRNA 5 | 5 | GCGGGCCAGCGCCGCCGGG | 5495 | CCCGGCGGCGCUGGCCCGC |
| siRNA 6 | 6 | CGGGCCAGCGCCGCCGGGA | 5496 | UCCCGGCGGCGCUGGCCCG |
| siRNA 7 | 7 | GGGCCAGCGCCGCCGGGAA | 5497 | UUCCCGGCGGCGCUGGCCC |
| siRNA 8 | 8 | GGCCAGCGCCGCCGGGAAA | 5498 | UUUCCCGGCGGCGCUGGCC |
| siRNA 9 | 9 | GCCAGCGCCGCCGGGAAAG | 5499 | CUUUCCCGGCGGCGCUGGC |
| siRNA 10 | 10 | CCAGCGCCGCCGGGAAAGG | 5500 | CCUUUCCCGGCGGCGCUGG |
| siRNA 11 | 11 | CAGCGCCGCCGGGAAAGGA | 5501 | UCCUUUCCCGGCGGCGCUG |
| siRNA 12 | 12 | AGCGCCGCCGGGAAAGGAG | 5502 | CUCCUUUCCCGGCGGCGCU |
| siRNA 13 | 13 | GCGCCGCCGGGAAAGGAGG | 5503 | CCUCCUUUCCCGGCGGCGC |
| siRNA 14 | 14 | CGCCGCCGGGAAAGGAGGU | 5504 | ACCUCCUUUCCCGGCGGCG |
| siRNA 15 | 15 | GCCGCCGGGAAAGGAGGUC | 5505 | GACCUCCUUUCCCGGCGGC |
| siRNA 16 | 16 | CCGCCGGGAAAGGAGGUCG | 5506 | CGACCUCCUUUCCCGGCGG |
| siRNA 17 | 17 | CGCCGGGAAAGGAGGUCGC | 5507 | GCGACCUCCUUUCCCGGCG |
| siRNA 18 | 18 | GCCGGGAAAGGAGGUCGCC | 5508 | GGCGACCUCCUUUCCCGGC |
| siRNA 19 | 19 | CCGGGAAAGGAGGUCGCCG | 5509 | CGGCGACCUCCUUUCCCGG |
| siRNA 20 | 20 | CGGGAAAGGAGGUCGCCGC | 5510 | GCGGCGACCUCCUUUCCCG |
| siRNA 21 | 21 | GGGAAAGGAGGUCGCCGCC | 5511 | GGCGGCGACCUCCUUUCCC |
| siRNA 22 | 22 | GGAAAGGAGGUCGCCGCCC | 5512 | GGGCGGCGACCUCCUUUCC |
| siRNA 23 | 23 | GAAAGGAGGUCGCCGCCCG | 5513 | CGGGCGGCGACCUCCUUUC |
| siRNA 24 | 24 | AAAGGAGGUCGCCGCCCGG | 5514 | CCGGGCGGCGACCUCCUUU |
| siRNA 25 | 25 | AAGGAGGUCGCCGCCCGGG | 5515 | CCCGGGCGGCGACCUCCUU |
| siRNA 26 | 26 | AGGAGGUCGCCGCCCGGGG | 5516 | CCCCGGGCGGCGACCUCCU |
| siRNA 27 | 27 | GGAGGUCGCCGCCCGGGGU | 5517 | ACCCCGGGCGGCGACCUCC |
| siRNA 28 | 28 | GAGGUCGCCGCCCGGGGUC | 5518 | GACCCCGGGCGGCGACCUC |
| siRNA 29 | 29 | AGGUCGCCGCCCGGGGUCG | 5519 | CGACCCCGGGCGGCGACCU |
| siRNA 30 | 30 | GGUCGCCGCCCGGGGUCGC | 5520 | GCGACCCCGGGCGGCGACC |
| siRNA 31 | 31 | GUCGCCGCCCGGGGUCGCC | 5521 | GGCGACCCCGGGCGGCGAC |
| siRNA 32 | 32 | UCGCCGCCCGGGGUCGCCC | 5522 | GGGCGACCCCGGGCGGCGA |
| siRNA 33 | 33 | CGCCGCCCGGGGUCGCCCG | 5523 | CGGGCGACCCCGGGCGGCG |
| siRNA 34 | 34 | GCCGCCCGGGGUCGCCCGG | 5524 | CCGGGCGACCCCGGGCGGC |
| siRNA 35 | 35 | CCGCCCGGGGUCGCCCGGG | 5525 | CCCGGGCGACCCCGGGCGG |
| siRNA 36 | 36 | CGCCCGGGGUCGCCCGGGC | 5526 | GCCCGGGCGACCCCGGGCG |
| siRNA 37 | 37 | GCCCGGGGUCGCCCGGGCU | 5527 | AGCCCGGGCGACCCCGGGC |
| siRNA 38 | 38 | CCCGGGGUCGCCCGGGCUU | 5528 | AAGCCCGGGCGACCCCGGG |
| siRNA 39 | 39 | CCGGGGUCGCCCGGGCUUG | 5529 | CAAGCCCGGGCGACCCCGG |
| siRNA 40 | 40 | CGGGGUCGCCCGGGCUUGC | 5530 | GCAAGCCCGGGCGACCCCG |
| siRNA 41 | 41 | GGGGUCGCCCGGGCUUGCG | 5531 | CGCAAGCCCGGGCGACCCC |
| siRNA 42 | 42 | GGGUCGCCCGGGCUUGCGG | 5532 | CCGCAAGCCCGGGCGACCC |
| siRNA 43 | 43 | GGUCGCCCGGGCUUGCGGG | 5533 | CCCGCAAGCCCGGGCGACC |
| siRNA 44 | 44 | GUCGCCCGGGCUUGCGGGU | 5534 | ACCCGCAAGCCCGGGCGAC |
| siRNA 45 | 45 | UCGCCCGGGCUUGCGGGUC | 5535 | GACCCGCAAGCCCGGGCGA |
| siRNA 46 | 46 | CGCCCGGGCUUGCGGGUCG | 5536 | CGACCCGCAAGCCCGGGCG |
| siRNA 47 | 47 | GCCCGGGCUUGCGGGUCGC | 5537 | GCGACCCGCAAGCCCGGGC |
| siRNA 48 | 48 | CCCGGGCUUGCGGGUCGCC | 5538 | GGCGACCCGCAAGCCCGGG |
| siRNA 49 | 49 | CCGGGCUUGCGGGUCGCCG | 5539 | CGGCGACCCGCAAGCCCGG |
| siRNA 50 | 50 | CGGGCUUGCGGGUCGCCGC | 5540 | GCGGCGACCCGCAAGCCCG |
| siRNA 51 | 51 | GGGCUUGCGGGUCGCCGCG | 5541 | CGCGGCGACCCGCAAGCCC |
| siRNA 52 | 52 | GGCUUGCGGGUCGCCGCGC | 5542 | GCGCGGCGACCCGCAAGCC |
| siRNA 53 | 53 | GCUUGCGGGUCGCCGCGCU | 5543 | AGCGCGGCGACCCGCAAGC |
| siRNA 54 | 54 | CUUGCGGGUCGCCGCGCUU | 5544 | AAGCGCGGCGACCCGCAAG |
| siRNA 55 | 55 | UUGCGGGUCGCCGCGCUUC | 5545 | GAAGCGCGGCGACCCGCAA |
| siRNA 56 | 56 | UGCGGGUCGCCGCGCUUCC | 5546 | GGAAGCGCGGCGACCCGCA |
| siRNA 57 | 57 | GCGGGUCGCCGCGCUUCCU | 5547 | AGGAAGCGCGGCGACCCGC |
| siRNA 58 | 58 | CGGGUCGCCGCGCUUCCUC | 5548 | GAGGAAGCGCGGCGACCCG |
| siRNA 59 | 59 | GGGUCGCCGCGCUUCCUCU | 5549 | AGAGGAAGCGCGGCGACCC |
| siRNA 60 | 60 | GGUCGCCGCGCUUCCUCUG | 5550 | CAGAGGAAGCGCGGCGACC |
| siRNA 61 | 61 | GUCGCCGCGCUUCCUCUGC | 5551 | GCAGAGGAAGCGCGGCGAC |
| siRNA 62 | 62 | UCGCCGCGCUUCCUCUGCG | 5552 | CGCAGAGGAAGCGCGGCGA |
| siRNA 63 | 63 | CGCCGCGCUUCCUCUGCGG | 5553 | CCGCAGAGGAAGCGCGGCG |
| siRNA 64 | 64 | GCCGCGCUUCCUCUGCGGG | 5554 | CCCGCAGAGGAAGCGCGGC |
| siRNA 65 | 65 | CCGCGCUUCCUCUGCGGGC | 5555 | GCCCGCAGAGGAAGCGCGG |
| siRNA 66 | 66 | CGCGCUUCCUCUGCGGGCG | 5556 | CGCCCGCAGAGGAAGCGCG |
| siRNA 67 | 67 | GCGCUUCCUCUGCGGGCGC | 5557 | GCGCCCGCAGAGGAAGCGC |
| siRNA 68 | 68 | CGCUUCCUCUGCGGGCGCU | 5558 | AGCGCCCGCAGAGGAAGCG |
| siRNA 69 | 69 | GCUUCCUCUGCGGGCGCUG | 5559 | CAGCGCCCGCAGAGGAAGC |
| siRNA 70 | 70 | CUUCCUCUGCGGGCGCUGC | 5560 | GCAGCGCCCGCAGAGGAAG |
| siRNA 71 | 71 | UUCCUCUGCGGGCGCUGCG | 5561 | CGCAGCGCCCGCAGAGGAA |
| siRNA 72 | 72 | UCCUCUGCGGGCGCUGCGG | 5562 | CCGCAGCGCCCGCAGAGGA |
| siRNA 73 | 73 | CCUCUGCGGGCGCUGCGGG | 5563 | CCCGCAGCGCCCGCAGAGG |
| siRNA 74 | 74 | CUCUGCGGGCGCUGCGGGA | 5564 | UCCCGCAGCGCCCGCAGAG |
| siRNA 75 | 75 | UCUGCGGGCGCUGCGGGAG | 5565 | CUCCCGCAGCGCCCGCAGA |
| siRNA 76 | 76 | CUGCGGGCGCUGCGGGAGG | 5566 | CCUCCCGCAGCGCCCGCAG |
| siRNA 77 | 77 | UGCGGGCGCUGCGGGAGGC | 5567 | GCCUCCCGCAGCGCCCGCA |
| siRNA 78 | 78 | GCGGGCGCUGCGGGAGGCG | 5568 | CGCCUCCCGCAGCGCCCGC |
| siRNA 79 | 79 | CGGGCGCUGCGGGAGGCGA | 5569 | UCGCCUCCCGCAGCGCCCG |
| siRNA 80 | 80 | GGGCGCUGCGGGAGGCGAG | 5570 | CUCGCCUCCCGCAGCGCCC |
| siRNA 81 | 81 | GGCGCUGCGGGAGGCGAGG | 5571 | CCUCGCCUCCCGCAGCGCC |
| siRNA 82 | 82 | GCGCUGCGGGAGGCGAGGG | 5572 | CCCUCGCCUCCCGCAGCGC |
| siRNA 83 | 83 | CGCUGCGGGAGGCGAGGGC | 5573 | GCCCUCGCCUCCCGCAGCG |
| siRNA 84 | 84 | GCUGCGGGAGGCGAGGGCC | 5574 | GGCCCUCGCCUCCCGCAGC |
| siRNA 85 | 85 | CUGCGGGAGGCGAGGGCCG | 5575 | CGGCCCUCGCCUCCCGCAG |
| siRNA 86 | 86 | UGCGGGAGGCGAGGGCCGC | 5576 | GCGGCCCUCGCCUCCCGCA |
| siRNA 87 | 87 | GCGGGAGGCGAGGGCCGCC | 5577 | GGCGGCCCUCGCCUCCCGC |
| siRNA 88 | 88 | CGGGAGGCGAGGGCCGCCU | 5578 | AGGCGGCCCUCGCCUCCCG |
| siRNA 89 | 89 | GGGAGGCGAGGGCCGCCUC | 5579 | GAGGCGGCCCUCGCCUCCC |
| siRNA 90 | 90 | GGAGGCGAGGGCCGCCUCG | 5580 | CGAGGCGGCCCUCGCCUCC |
| siRNA 91 | 91 | GAGGCGAGGGCCGCCUCGG | 5581 | CCGAGGCGGCCCUCGCCUC |
| siRNA 92 | 92 | AGGCGAGGGCCGCCUCGGC | 5582 | GCCGAGGCGGCCCUCGCCU |
| siRNA 93 | 93 | GGCGAGGGCCGCCUCGGCG | 5583 | CGCCGAGGCGGCCCUCGCC |
| siRNA 94 | 94 | GCGAGGGCCGCCUCGGCGG | 5584 | CCGCCGAGGCGGCCCUCGC |
| siRNA 95 | 95 | CGAGGGCCGCCUCGGCGGA | 5585 | UCCGCCGAGGCGGCCCUCG |
| siRNA 96 | 96 | GAGGGCCGCCUCGGCGGAG | 5586 | CUCCGCCGAGGCGGCCCUC |
| siRNA 97 | 97 | AGGGCCGCCUCGGCGGAGG | 5587 | CCUCCGCCGAGGCGGCCCU |
| siRNA 98 | 98 | GGGCCGCCUCGGCGGAGGA | 5588 | UCCUCCGCCGAGGCGGCCC |
| siRNA 99 | 99 | GGCCGCCUCGGCGGAGGAA | 5589 | UUCCUCCGCCGAGGCGGCC |
| siRNA 100 | 100 | GCCGCCUCGGCGGAGGAAG | 5590 | CUUCCUCCGCCGAGGCGGC |
| siRNA 101 | 101 | CCGCCUCGGCGGAGGAAGC | 5591 | GCUUCCUCCGCCGAGGCGG |
| siRNA 102 | 102 | CGCCUCGGCGGAGGAAGCC | 5592 | GGCUUCCUCCGCCGAGGCG |
| siRNA 103 | 103 | GCCUCGGCGGAGGAAGCCG | 5593 | CGGCUUCCUCCGCCGAGGC |
| siRNA 104 | 104 | CCUCGGCGGAGGAAGCCGA | 5594 | UCGGCUUCCUCCGCCGAGG |
| siRNA 105 | 105 | CUCGGCGGAGGAAGCCGAC | 5595 | GUCGGCUUCCUCCGCCGAG |
| siRNA 106 | 106 | UCGGCGGAGGAAGCCGACC | 5596 | GGUCGGCUUCCUCCGCCGA |
| siRNA 107 | 107 | CGGCGGAGGAAGCCGACCC | 5597 | GGGUCGGCUUCCUCCGCCG |
| siRNA 108 | 108 | GGCGGAGGAAGCCGACCCC | 5598 | GGGGUCGGCUUCCUCCGCC |
| siRNA 109 | 109 | GCGGAGGAAGCCGACCCCG | 5599 | CGGGGUCGGCUUCCUCCGC |
| siRNA 110 | 110 | CGGAGGAAGCCGACCCCGG | 5600 | CCGGGGUCGGCUUCCUCCG |
| siRNA 111 | 111 | GGAGGAAGCCGACCCCGGG | 5601 | CCCGGGGUCGGCUUCCUCC |
| siRNA 112 | 112 | GAGGAAGCCGACCCCGGGG | 5602 | CCCCGGGGUCGGCUUCCUC |
| siRNA 113 | 113 | AGGAAGCCGACCCCGGGGU | 5603 | ACCCCGGGGUCGGCUUCCU |
| siRNA 114 | 114 | GGAAGCCGACCCCGGGGUC | 5604 | GACCCCGGGGUCGGCUUCC |
| siRNA 115 | 115 | GAAGCCGACCCCGGGGUCU | 5605 | AGACCCCGGGGUCGGCUUC |
| siRNA 116 | 116 | AAGCCGACCCCGGGGUCUC | 5606 | GAGACCCCGGGGUCGGCUU |
| siRNA 117 | 117 | AGCCGACCCCGGGGUCUCC | 5607 | GGAGACCCCGGGGUCGGCU |
| siRNA 118 | 118 | GCCGACCCCGGGGUCUCCG | 5608 | CGGAGACCCCGGGGUCGGC |
| siRNA 119 | 119 | CCGACCCCGGGGUCUCCGC | 5609 | GCGGAGACCCCGGGGUCGG |
| siRNA 120 | 120 | CGACCCCGGGGUCUCCGCC | 5610 | GGCGGAGACCCCGGGGUCG |
| siRNA 121 | 121 | GACCCCGGGGUCUCCGCCA | 5611 | UGGCGGAGACCCCGGGGUC |
| siRNA 122 | 122 | ACCCCGGGGUCUCCGCCAG | 5612 | CUGGCGGAGACCCCGGGGU |
| siRNA 123 | 123 | CCCCGGGGUCUCCGCCAGC | 5613 | GCUGGCGGAGACCCCGGGG |
| siRNA 124 | 124 | CCCGGGGUCUCCGCCAGCC | 5614 | GGCUGGCGGAGACCCCGGG |
| siRNA 125 | 125 | CCGGGGUCUCCGCCAGCCC | 5615 | GGGCUGGCGGAGACCCCGG |
| siRNA 126 | 126 | CGGGGUCUCCGCCAGCCCG | 5616 | CGGGCUGGCGGAGACCCCG |
| siRNA 127 | 127 | GGGGUCUCCGCCAGCCCGC | 5617 | GCGGGCUGGCGGAGACCCC |
| siRNA 128 | 128 | GGGUCUCCGCCAGCCCGCC | 5618 | GGCGGGCUGGCGGAGACCC |
| siRNA 129 | 129 | GGUCUCCGCCAGCCCGCCG | 5619 | CGGCGGGCUGGCGGAGACC |
| siRNA 130 | 130 | GUCUCCGCCAGCCCGCCGG | 5620 | CCGGCGGGCUGGCGGAGAC |
| siRNA 131 | 131 | UCUCCGCCAGCCCGCCGGC | 5621 | GCCGGCGGGCUGGCGGAGA |
| siRNA 132 | 132 | CUCCGCCAGCCCGCCGGCG | 5622 | CGCCGGCGGGCUGGCGGAG |
| siRNA 133 | 133 | UCCGCCAGCCCGCCGGCGC | 5623 | GCGCCGGCGGGCUGGCGGA |
| siRNA 134 | 134 | CCGCCAGCCCGCCGGCGCC | 5624 | GGCGCCGGCGGGCUGGCGG |
| siRNA 135 | 135 | CGCCAGCCCGCCGGCGCCG | 5625 | CGGCGCCGGCGGGCUGGCG |
| siRNA 136 | 136 | GCCAGCCCGCCGGCGCCGC | 5626 | GCGGCGCCGGCGGGCUGGC |
| siRNA 137 | 137 | CCAGCCCGCCGGCGCCGCC | 5627 | GGCGGCGCCGGCGGGCUGG |
| siRNA 138 | 138 | CAGCCCGCCGGCGCCGCCU | 5628 | AGGCGGCGCCGGCGGGCUG |
| siRNA 139 | 139 | AGCCCGCCGGCGCCGCCUC | 5629 | GAGGCGGCGCCGGCGGGCU |
| siRNA 140 | 140 | GCCCGCCGGCGCCGCCUCC | 5630 | GGAGGCGGCGCCGGCGGGC |
| siRNA 141 | 141 | CCCGCCGGCGCCGCCUCCG | 5631 | CGGAGGCGGCGCCGGCGGG |
| siRNA 142 | 142 | CCGCCGGCGCCGCCUCCGA | 5632 | UCGGAGGCGGCGCCGGCGG |
| siRNA 143 | 143 | CGCCGGCGCCGCCUCCGAG | 5633 | CUCGGAGGCGGCGCCGGCG |
| siRNA 144 | 144 | GCCGGCGCCGCCUCCGAGC | 5634 | GCUCGGAGGCGGCGCCGGC |
| siRNA 145 | 145 | CCGGCGCCGCCUCCGAGCC | 5635 | GGCUCGGAGGCGGCGCCGG |
| siRNA 146 | 146 | CGGCGCCGCCUCCGAGCCG | 5636 | CGGCUCGGAGGCGGCGCCG |
| siRNA 147 | 147 | GGCGCCGCCUCCGAGCCGC | 5637 | GCGGCUCGGAGGCGGCGCC |
| siRNA 148 | 148 | GCGCCGCCUCCGAGCCGCC | 5638 | GGCGGCUCGGAGGCGGCGC |
| siRNA 149 | 149 | CGCCGCCUCCGAGCCGCCU | 5639 | AGGCGGCUCGGAGGCGGCG |
| siRNA 150 | 150 | GCCGCCUCCGAGCCGCCUC | 5640 | GAGGCGGCUCGGAGGCGGC |
| siRNA 151 | 151 | CCGCCUCCGAGCCGCCUCG | 5641 | CGAGGCGGCUCGGAGGCGG |
| siRNA 152 | 152 | CGCCUCCGAGCCGCCUCGG | 5642 | CCGAGGCGGCUCGGAGGCG |
| siRNA 153 | 153 | GCCUCCGAGCCGCCUCGGC | 5643 | GCCGAGGCGGCUCGGAGGC |
| siRNA 154 | 154 | CCUCCGAGCCGCCUCGGCC | 5644 | GGCCGAGGCGGCUCGGAGG |
| siRNA 155 | 155 | CUCCGAGCCGCCUCGGCCG | 5645 | CGGCCGAGGCGGCUCGGAG |
| siRNA 156 | 156 | UCCGAGCCGCCUCGGCCGU | 5646 | ACGGCCGAGGCGGCUCGGA |
| siRNA 157 | 157 | CCGAGCCGCCUCGGCCGUA | 5647 | UACGGCCGAGGCGGCUCGG |
| siRNA 158 | 158 | CGAGCCGCCUCGGCCGUAG | 5648 | CUACGGCCGAGGCGGCUCG |
| siRNA 159 | 159 | GAGCCGCCUCGGCCGUAGC | 5649 | GCUACGGCCGAGGCGGCUC |
| siRNA 160 | 160 | AGCCGCCUCGGCCGUAGCC | 5650 | GGCUACGGCCGAGGCGGCU |
| siRNA 161 | 161 | GCCGCCUCGGCCGUAGCCU | 5651 | AGGCUACGGCCGAGGCGGC |
| siRNA 162 | 162 | CCGCCUCGGCCGUAGCCUC | 5652 | GAGGCUACGGCCGAGGCGG |
| siRNA 163 | 163 | CGCCUCGGCCGUAGCCUCG | 5653 | CGAGGCUACGGCCGAGGCG |
| siRNA 164 | 164 | GCCUCGGCCGUAGCCUCGG | 5654 | CCGAGGCUACGGCCGAGGC |
| siRNA 165 | 165 | CCUCGGCCGUAGCCUCGGC | 5655 | GCCGAGGCUACGGCCGAGG |
| siRNA 166 | 166 | CUCGGCCGUAGCCUCGGCC | 5656 | GGCCGAGGCUACGGCCGAG |
| siRNA 167 | 167 | UCGGCCGUAGCCUCGGCCC | 5657 | GGGCCGAGGCUACGGCCGA |
| siRNA 168 | 168 | CGGCCGUAGCCUCGGCCCG | 5658 | CGGGCCGAGGCUACGGCCG |
| siRNA 169 | 169 | GGCCGUAGCCUCGGCCCGG | 5659 | CCGGGCCGAGGCUACGGCC |
| siRNA 170 | 170 | GCCGUAGCCUCGGCCCGGG | 5660 | CCCGGGCCGAGGCUACGGC |
| siRNA 171 | 171 | CCGUAGCCUCGGCCCGGGC | 5661 | GCCCGGGCCGAGGCUACGG |
| siRNA 172 | 172 | CGUAGCCUCGGCCCGGGCG | 5662 | CGCCCGGGCCGAGGCUACG |
| siRNA 173 | 173 | GUAGCCUCGGCCCGGGCGG | 5663 | CCGCCCGGGCCGAGGCUAC |
| siRNA 174 | 174 | UAGCCUCGGCCCGGGGGG | 5664 | CCCGCCCGGGCCGAGGCUA |
| siRNA 175 | 175 | AGCCUCGGCCCGGGCGGGA | 5665 | UCCCGCCCGGGCCGAGGCU |
| siRNA 176 | 176 | GCCUCGGCCCGGGCGGGAG | 5666 | CUCCCGCCCGGGCCGAGGC |
| siRNA 177 | 177 | CCUCGGCCCGGGCGGGAGG | 5667 | CCUCCCGCCCGGGCCGAGG |
| siRNA 178 | 178 | CUCGGCCCGGGCGGGAGGC | 5668 | GCCUCCCGCCCGGGCCGAG |
| siRNA 179 | 179 | UCGGCCCGGGCGGGAGGCG | 5669 | CGCCUCCCGCCCGGGCCGA |
| siRNA 180 | 180 | CGGCCCGGGCGGGAGGCGG | 5670 | CCGCCUCCCGCCCGGGCCG |
| siRNA 181 | 181 | GGCCCGGGCGGGAGGCGGC | 5671 | GCCGCCUCCCGCCCGGGCC |
| siRNA 182 | 182 | GCCCGGGCGGGAGGCGGCG | 5672 | CGCCGCCUCCCGCCCGGGC |
| siRNA 183 | 183 | CCCGGGCGGGAGGCGGCGG | 5673 | CCGCCGCCUCCCGCCCGGG |
| siRNA 184 | 184 | CCGGGCGGGAGGCGGCGGC | 5674 | GCCGCCGCCUCCCGCCCGG |
| siRNA 185 | 185 | CGGGCGGGAGGCGGCGGCC | 5675 | GGCCGCCGCCUCCCGCCCG |
| siRNA 186 | 186 | GGGCGGGAGGCGGCGGCCG | 5676 | CGGCCGCCGCCUCCCGCCC |
| siRNA 187 | 187 | GGCGGGAGGCGGCGGCCGC | 5677 | GCGGCCGCCGCCUCCCGCC |
| siRNA 188 | 188 | GCGGGAGGCGGCGGCCGCC | 5678 | GGCGGCCGCCGCCUCCCGC |
| siRNA 189 | 189 | CGGGAGGCGGCGGCCGCCG | 5679 | CGGCGGCCGCCGCCUCCCG |
| siRNA 190 | 190 | GGGAGGCGGCGGCCGCCGG | 5680 | CCGGCGGCCGCCGCCUCCC |
| siRNA 191 | 191 | GGAGGCGGCGGCCGCCGGC | 5681 | GCCGGCGGCCGCCGCCUCC |
| siRNA 192 | 192 | GAGGCGGCGGCCGCCGGCG | 5682 | CGCCGGCGGCCGCCGCCUC |
| siRNA 193 | 193 | AGGCGGCGGCCGCCGGCGA | 5683 | UCGCCGGCGGCCGCCGCCU |
| siRNA 194 | 194 | GGCGGCGGCCGCCGGCGAG | 5684 | CUCGCCGGCGGCCGCCGCC |
| siRNA 195 | 195 | GCGGCGGCCGCCGGCGAGG | 5685 | CCUCGCCGGCGGCCGCCGC |
| siRNA 196 | 196 | CGGCGGCCGCCGGCGAGGC | 5686 | GCCUCGCCGGCGGCCGCCG |
| siRNA 197 | 197 | GGCGGCCGCCGGCGAGGCG | 5687 | CGCCUCGCCGGCGGCCGCC |
| siRNA 198 | 198 | GCGGCCGCCGGCGAGGCGA | 5688 | UCGCCUCGCCGGCGGCCGC |
| siRNA 199 | 199 | CGGCCGCCGGCGAGGCGAG | 5689 | CUCGCCUCGCCGGCGGCCG |
| siRNA 200 | 200 | GGCCGCCGGCGAGGCGAGG | 5690 | CCUCGCCUCGCCGGCGGCC |
| siRNA 201 | 201 | GCCGCCGGCGAGGCGAGGC | 5691 | GCCUCGCCUCGCCGGCGGC |
| siRNA 202 | 202 | CCGCCGGCGAGGCGAGGCG | 5692 | CGCCUCGCCUCGCCGGCGG |
| siRNA 203 | 203 | CGCCGGCGAGGCGAGGCGG | 5693 | CCGCCUCGCCUCGCCGGCG |
| siRNA 204 | 204 | GCCGGCGAGGCGAGGCGGC | 5694 | GCCGCCUCGCCUCGCCGGC |
| siRNA 205 | 205 | CCGGCGAGGCGAGGCGGCC | 5695 | GGCCGCCUCGCCUCGCCGG |
| siRNA 206 | 206 | CGGCGAGGCGAGGCGGCCC | 5696 | GGGCCGCCUCGCCUCGCCG |
| siRNA 207 | 207 | GGCGAGGCGAGGCGGCCCG | 5697 | CGGGCCGCCUCGCCUCGCC |
| siRNA 208 | 208 | GCGAGGCGAGGCGGCCCGC | 5698 | GCGGGCCGCCUCGCCUCGC |
| siRNA 209 | 209 | CGAGGCGAGGCGGCCCGCG | 5699 | CGCGGGCCGCCUCGCCUCG |
| siRNA 210 | 210 | GAGGCGAGGCGGCCCGCGC | 5700 | GCGCGGGCCGCCUCGCCUC |
| siRNA 211 | 211 | AGGCGAGGCGGCCCGCGCC | 5701 | GGCGCGGGCCGCCUCGCCU |
| siRNA 212 | 212 | GGCGAGGCGGCCCGCGCCC | 5702 | GGGCGCGGGCCGCCUCGCC |
| siRNA 213 | 213 | GCGAGGCGGCCCGCGCCCU | 5703 | AGGGCGCGGGCCGCCUCGC |
| siRNA 214 | 214 | CGAGGCGGCCCGCGCCCUG | 5704 | CAGGGCGCGGGCCGCCUCG |
| siRNA 215 | 215 | GAGGCGGCCCGCGCCCUGC | 5705 | GCAGGGCGCGGGCCGCCUC |
| siRNA 216 | 216 | AGGCGGCCCGCGCCCUGCC | 5706 | GGCAGGGCGCGGGCCGCCU |
| siRNA 217 | 217 | GGCGGCCCGCGCCCUGCCU | 5707 | AGGCAGGGCGCGGGCCGCC |
| siRNA 218 | 218 | GCGGCCCGCGCCCUGCCUG | 5708 | CAGGCAGGGCGCGGGCCGC |
| siRNA 219 | 219 | CGGCCCGCGCCCUGCCUGU | 5709 | ACAGGCAGGGCGCGGGCCG |
| siRNA 220 | 220 | GGCCCGCGCCCUGCCUGUC | 5710 | GACAGGCAGGGCGCGGGCC |
| siRNA 221 | 221 | GCCCGCGCCCUGCCUGUCA | 5711 | UGACAGGCAGGGCGCGGGC |
| siRNA 222 | 222 | CCCGCGCCCUGCCUGUCAG | 5712 | CUGACAGGCAGGGCGCGGG |
| siRNA 223 | 223 | CCGCGCCCUGCCUGUCAGG | 5713 | CCUGACAGGCAGGGCGCGG |
| siRNA 224 | 224 | CGCGCCCUGCCUGUCAGGC | 5714 | GCCUGACAGGCAGGGCGCG |
| siRNA 225 | 225 | GCGCCCUGCCUGUCAGGCC | 5715 | GGCCUGACAGGCAGGGCGC |
| siRNA 226 | 226 | CGCCCUGCCUGUCAGGCCA | 5716 | UGGCCUGACAGGCAGGGCG |
| siRNA 227 | 227 | GCCCUGCCUGUCAGGCCAC | 5717 | GUGGCCUGACAGGCAGGGC |
| siRNA 228 | 228 | CCCUGCCUGUCAGGCCACC | 5718 | GGUGGCCUGACAGGCAGGG |
| siRNA 229 | 229 | CCUGCCUGUCAGGCCACCG | 5719 | CGGUGGCCUGACAGGCAGG |
| siRNA 230 | 230 | CUGCCUGUCAGGCCACCGG | 5720 | CCGGUGGCCUGACAGGCAG |
| siRNA 231 | 231 | UGCCUGUCAGGCCACCGGC | 5721 | GCCGGUGGCCUGACAGGCA |
| siRNA 232 | 232 | GCCUGUCAGGCCACCGGCC | 5722 | GGCCGGUGGCCUGACAGGC |
| siRNA 233 | 233 | CCUGUCAGGCCACCGGCCC | 5723 | GGGCCGGUGGCCUGACAGG |
| siRNA 234 | 234 | CUGUCAGGCCACCGGCCCG | 5724 | CGGGCCGGUGGCCUGACAG |
| siRNA 235 | 235 | UGUCAGGCCACCGGCCCGG | 5725 | CCGGGCCGGUGGCCUGACA |
| siRNA 236 | 236 | GUCAGGCCACCGGCCCGGC | 5726 | GCCGGGCCGGUGGCCUGAC |
| siRNA 237 | 237 | UCAGGCCACCGGCCCGGCC | 5727 | GGCCGGGCCGGUGGCCUGA |
| siRNA 238 | 238 | CAGGCCACCGGCCCGGCCC | 5728 | GGGCCGGGCCGGUGGCCUG |
| siRNA 239 | 239 | AGGCCACCGGCCCGGCCCG | 5729 | CGGGCCGGGCCGGUGGCCU |
| siRNA 240 | 240 | GGCCACCGGCCCGGCCCGC | 5730 | GCGGGCCGGGCCGGUGGCC |
| siRNA 241 | 241 | GCCACCGGCCCGGCCCGCG | 5731 | CGCGGGCCGGGCCGGUGGC |
| siRNA 242 | 242 | CCACCGGCCCGGCCCGCGG | 5732 | CCGCGGGCCGGGCCGGUGG |
| siRNA 243 | 243 | CACCGGCCCGGCCCGCGGG | 5733 | CCCGCGGGCCGGGCCGGUG |
| siRNA 244 | 244 | ACCGGCCCGGCCCGCGGGC | 5734 | GCCCGCGGGCCGGGCCGGU |
| siRNA 245 | 245 | CCGGCCCGGCCCGCGGGCU | 5735 | AGCCCGCGGGCCGGGCCGG |
| siRNA 246 | 246 | CGGCCCGGCCCGCGGGCUG | 5736 | CAGCCCGCGGGCCGGGCCG |
| siRNA 247 | 247 | GGCCCGGCCCGCGGGCUGC | 5737 | GCAGCCCGCGGGCCGGGCC |
| siRNA 248 | 248 | GCCCGGCCCGCGGGCUGCC | 5738 | GGCAGCCCGCGGGCCGGGC |
| siRNA 249 | 249 | CCCGGCCCGCGGGCUGCCC | 5739 | GGGCAGCCCGCGGGCCGGG |
| siRNA 250 | 250 | CCGGCCCGCGGGCUGCCCC | 5740 | GGGGCAGCCCGCGGGCCGG |
| siRNA 251 | 251 | CGGCCCGCGGGCUGCCCCC | 5741 | GGGGGCAGCCCGCGGGCCG |
| siRNA 252 | 252 | GGCCCGCGGGCUGCCCCCG | 5742 | CGGGGGCAGCCCGCGGGCC |
| siRNA 253 | 253 | GCCCGCGGGCUGCCCCCGA | 5743 | UCGGGGGCAGCCCGCGGGC |
| siRNA 254 | 254 | CCCGCGGGCUGCCCCCGAU | 5744 | AUCGGGGGCAGCCCGCGGG |
| siRNA 255 | 255 | CCGCGGGCUGCCCCCGAUG | 5745 | CAUCGGGGGCAGCCCGCGG |
| siRNA 256 | 256 | CGCGGGCUGCCCCCGAUGC | 5746 | GCAUCGGGGGCAGCCCGCG |
| siRNA 257 | 257 | GCGGGCUGCCCCCGAUGCG | 5747 | CGCAUCGGGGGCAGCCCGC |
| siRNA 258 | 258 | CGGGCUGCCCCCGAUGCGG | 5748 | CCGCAUCGGGGGCAGCCCG |
| siRNA 259 | 259 | GGGCUGCCCCCGAUGCGGA | 5749 | UCCGCAUCGGGGGCAGCCC |
| siRNA 260 | 260 | GGCUGCCCCCGAUGCGGAG | 5750 | CUCCGCAUCGGGGGCAGCC |
| siRNA 261 | 261 | GCUGCCCCCGAUGCGGAGG | 5751 | CCUCCGCAUCGGGGGCAGC |
| siRNA 262 | 262 | CUGCCCCCGAUGCGGAGGC | 5752 | GCCUCCGCAUCGGGGGCAG |
| siRNA 263 | 263 | UGCCCCCGAUGCGGAGGCG | 5753 | CGCCUCCGCAUCGGGGGCA |
| siRNA 264 | 264 | GCCCCCGAUGCGGAGGCGC | 5754 | GCGCCUCCGCAUCGGGGGC |
| siRNA 265 | 265 | CCCCCGAUGCGGAGGCGCU | 5755 | AGCGCCUCCGCAUCGGGGG |
| siRNA 266 | 266 | CCCCGAUGCGGAGGCGCUG | 5756 | CAGCGCCUCCGCAUCGGGG |
| siRNA 267 | 267 | CCCGAUGCGGAGGCGCUGU | 5757 | ACAGCGCCUCCGCAUCGGG |
| siRNA 268 | 268 | CCGAUGCGGAGGCGCUGUC | 5758 | GACAGCGCCUCCGCAUCGG |
| siRNA 269 | 269 | CGAUGCGGAGGCGCUGUCG | 5759 | CGACAGCGCCUCCGCAUCG |
| siRNA 270 | 270 | GAUGCGGAGGCGCUGUCGC | 5760 | GCGACAGCGCCUCCGCAUC |
| siRNA 271 | 271 | AUGCGGAGGCGCUGUCGCC | 5761 | GGCGACAGCGCCUCCGCAU |
| siRNA 272 | 272 | UGCGGAGGCGCUGUCGCCG | 5762 | CGGCGACAGCGCCUCCGCA |
| siRNA 273 | 273 | GCGGAGGCGCUGUCGCCGG | 5763 | CCGGCGACAGCGCCUCCGC |
| siRNA 274 | 274 | CGGAGGCGCUGUCGCCGGG | 5764 | CCCGGCGACAGCGCCUCCG |
| siRNA 275 | 275 | GGAGGCGCUGUCGCCGGGG | 5765 | CCCCGGCGACAGCGCCUCC |
| siRNA 276 | 276 | GAGGCGCUGUCGCCGGGGC | 5766 | GCCCCGGCGACAGCGCCUC |
| siRNA 277 | 277 | AGGCGCUGUCGCCGGGGCC | 5767 | GGCCCCGGCGACAGCGCCU |
| siRNA 278 | 278 | GGCGCUGUCGCCGGGGCCA | 5768 | UGGCCCCGGCGACAGCGCC |
| siRNA 279 | 279 | GCGCUGUCGCCGGGGCCAU | 5769 | AUGGCCCCGGCGACAGCGC |
| siRNA 280 | 280 | CGCUGUCGCCGGGGCCAUG | 5770 | CAUGGCCCCGGCGACAGCG |
| siRNA 281 | 281 | GCUGUCGCCGGGGCCAUGC | 5771 | GCAUGGCCCCGGCGACAGC |
| siRNA 282 | 282 | CUGUCGCCGGGGCCAUGCA | 5772 | UGCAUGGCCCCGGCGACAG |
| siRNA 283 | 283 | UGUCGCCGGGGCCAUGCAG | 5773 | CUGCAUGGCCCCGGCGACA |
| siRNA 284 | 284 | GUCGCCGGGGCCAUGCAGC | 5774 | GCUGCAUGGCCCCGGCGAC |
| siRNA 285 | 285 | UCGCCGGGGCCAUGCAGCA | 5775 | UGCUGCAUGGCCCCGGCGA |
| siRNA 286 | 286 | CGCCGGGGCCAUGCAGCAG | 5776 | CUGCUGCAUGGCCCCGGCG |
| siRNA 287 | 287 | GCCGGGGCCAUGCAGCAGG | 5777 | CCUGCUGCAUGGCCCCGGC |
| siRNA 288 | 288 | CCGGGGCCAUGCAGCAGGC | 5778 | GCCUGCUGCAUGGCCCCGG |
| siRNA 289 | 289 | CGGGGCCAUGCAGCAGGCG | 5779 | CGCCUGCUGCAUGGCCCCG |
| siRNA 290 | 290 | GGGGCCAUGCAGCAGGCGC | 5780 | GCGCCUGCUGCAUGGCCCC |
| siRNA 291 | 291 | GGGCCAUGCAGCAGGCGCC | 5781 | GGCGCCUGCUGCAUGGCCC |
| siRNA 292 | 292 | GGCCAUGCAGCAGGCGCCG | 5782 | CGGCGCCUGCUGCAUGGCC |
| siRNA 293 | 293 | GCCAUGCAGCAGGCGCCGC | 5783 | GCGGCGCCUGCUGCAUGGC |
| siRNA 294 | 294 | CCAUGCAGCAGGCGCCGCA | 5784 | UGCGGCGCCUGCUGCAUGG |
| siRNA 295 | 295 | CAUGCAGCAGGCGCCGCAG | 5785 | CUGCGGCGCCUGCUGCAUG |
| siRNA 296 | 296 | AUGCAGCAGGCGCCGCAGC | 5786 | GCUGCGGCGCCUGCUGCAU |
| siRNA 297 | 297 | UGCAGCAGGCGCCGCAGCC | 5787 | GGCUGCGGCGCCUGCUGCA |
| siRNA 298 | 298 | GCAGCAGGCGCCGCAGCCU | 5788 | AGGCUGCGGCGCCUGCUGC |
| siRNA 299 | 299 | CAGCAGGCGCCGCAGCCUU | 5789 | AAGGCUGCGGCGCCUGCUG |
| siRNA 300 | 300 | AGCAGGCGCCGCAGCCUUA | 5790 | UAAGGCUGCGGCGCCUGCU |
| siRNA 301 | 301 | GCAGGCGCCGCAGCCUUAC | 5791 | GUAAGGCUGCGGCGCCUGC |
| siRNA 302 | 302 | CAGGCGCCGCAGCCUUACG | 5792 | CGUAAGGCUGCGGCGCCUG |
| siRNA 303 | 303 | AGGCGCCGCAGCCUUACGA | 5793 | UCGUAAGGCUGCGGCGCCU |
| siRNA 304 | 304 | GGCGCCGCAGCCUUACGAG | 5794 | CUCGUAAGGCUGCGGCGCC |
| siRNA 305 | 305 | GCGCCGCAGCCUUACGAGU | 5795 | ACUCGUAAGGCUGCGGCGC |
| siRNA 306 | 306 | CGCCGCAGCCUUACGAGUU | 5796 | AACUCGUAAGGCUGCGGCG |
| siRNA 307 | 307 | GCCGCAGCCUUACGAGUUC | 5797 | GAACUCGUAAGGCUGCGGC |
| siRNA 308 | 308 | CCGCAGCCUUACGAGUUCU | 5798 | AGAACUCGUAAGGCUGCGG |
| siRNA 309 | 309 | CGCAGCCUUACGAGUUCUU | 5799 | AAGAACUCGUAAGGCUGCG |
| siRNA 310 | 310 | GCAGCCUUACGAGUUCUUC | 5800 | GAAGAACUCGUAAGGCUGC |
| siRNA 311 | 311 | CAGCCUUACGAGUUCUUCA | 5801 | UGAAGAACUCGUAAGGCUG |
| siRNA 312 | 312 | AGCCUUACGAGUUCUUCAG | 5802 | CUGAAGAACUCGUAAGGCU |
| siRNA 313 | 313 | GCCUUACGAGUUCUUCAGC | 5803 | GCUGAAGAACUCGUAAGGC |
| siRNA 314 | 314 | CCUUACGAGUUCUUCAGCG | 5804 | CGCUGAAGAACUCGUAAGG |
| siRNA 315 | 315 | CUUACGAGUUCUUCAGCGA | 5805 | UCGCUGAAGAACUCGUAAG |
| siRNA 316 | 316 | UUACGAGUUCUUCAGCGAG | 5806 | CUCGCUGAAGAACUCGUAA |
| siRNA 317 | 317 | UACGAGUUCUUCAGCGAGG | 5807 | CCUCGCUGAAGAACUCGUA |
| siRNA 318 | 318 | ACGAGUUCUUCAGCGAGGA | 5808 | UCCUCGCUGAAGAACUCGU |
| siRNA 319 | 319 | CGAGUUCUUCAGCGAGGAG | 5809 | CUCCUCGCUGAAGAACUCG |
| siRNA 320 | 320 | GAGUUCUUCAGCGAGGAGA | 5810 | UCUCCUCGCUGAAGAACUC |
| siRNA 321 | 321 | AGUUCUUCAGCGAGGAGAA | 5811 | UUCUCCUCGCUGAAGAACU |
| siRNA 322 | 322 | GUUCUUCAGCGAGGAGAAC | 5812 | GUUCUCCUCGCUGAAGAAC |
| siRNA 323 | 323 | UUCUUCAGCGAGGAGAACA | 5813 | UGUUCUCCUCGCUGAAGAA |
| siRNA 324 | 324 | UCUUCAGCGAGGAGAACAG | 5814 | CUGUUCUCCUCGCUGAAGA |
| siRNA 325 | 325 | CUUCAGCGAGGAGAACAGU | 5815 | ACUGUUCUCCUCGCUGAAG |
| siRNA 326 | 326 | UUCAGCGAGGAGAACAGUC | 5816 | GACUGUUCUCCUCGCUGAA |
| siRNA 327 | 327 | UCAGCGAGGAGAACAGUCC | 5817 | GGACUGUUCUCCUCGCUGA |
| siRNA 328 | 328 | CAGCGAGGAGAACAGUCCG | 5818 | CGGACUGUUCUCCUCGCUG |
| siRNA 329 | 329 | AGCGAGGAGAACAGUCCGA | 5819 | UCGGACUGUUCUCCUCGCU |
| siRNA 330 | 330 | GCGAGGAGAACAGUCCGAA | 5820 | UUCGGACUGUUCUCCUCGC |
| siRNA 331 | 331 | CGAGGAGAACAGUCCGAAA | 5821 | UUUCGGACUGUUCUCCUCG |
| siRNA 332 | 332 | GAGGAGAACAGUCCGAAAU | 5822 | AUUUCGGACUGUUCUCCUC |
| siRNA 333 | 333 | AGGAGAACAGUCCGAAAUG | 5823 | CAUUUCGGACUGUUCUCCU |
| siRNA 334 | 334 | GGAGAACAGUCCGAAAUGG | 5824 | CCAUUUCGGACUGUUCUCC |
| siRNA 335 | 335 | GAGAACAGUCCGAAAUGGC | 5825 | GCCAUUUCGGACUGUUCUC |
| siRNA 336 | 336 | AGAACAGUCCGAAAUGGCG | 5826 | CGCCAUUUCGGACUGUUCU |
| siRNA 337 | 337 | GAACAGUCCGAAAUGGCGG | 5827 | CCGCCAUUUCGGACUGUUC |
| siRNA 338 | 338 | AACAGUCCGAAAUGGCGGG | 5828 | CCCGCCAUUUCGGACUGUU |
| siRNA 339 | 339 | ACAGUCCGAAAUGGCGGGG | 5829 | CCCCGCCAUUUCGGACUGU |
| siRNA 340 | 340 | CAGUCCGAAAUGGCGGGGA | 5830 | UCCCCGCCAUUUCGGACUG |
| siRNA 341 | 341 | AGUCCGAAAUGGCGGGGAC | 5831 | GUCCCCGCCAUUUCGGACU |
| siRNA 342 | 342 | GUCCGAAAUGGCGGGGACU | 5832 | AGUCCCCGCCAUUUCGGAC |
| siRNA 343 | 343 | UCCGAAAUGGCGGGGACUG | 5833 | CAGUCCCCGCCAUUUCGGA |
| siRNA 344 | 344 | CCGAAAUGGCGGGGACUGU | 5834 | ACAGUCCCCGCCAUUUCGG |
| siRNA 345 | 345 | CGAAAUGGCGGGGACUGUU | 5835 | AACAGUCCCCGCCAUUUCG |
| siRNA 346 | 346 | GAAAUGGCGGGGACUGUUG | 5836 | CAACAGUCCCCGCCAUUUC |
| siRNA 347 | 347 | AAAUGGCGGGGACUGUUGG | 5837 | CCAACAGUCCCCGCCAUUU |
| siRNA 348 | 348 | AAUGGCGGGGACUGUUGGU | 5838 | ACCAACAGUCCCCGCCAUU |
| siRNA 349 | 349 | AUGGCGGGGACUGUUGGUC | 5839 | GACCAACAGUCCCCGCCAU |
| siRNA 350 | 350 | UGGCGGGGACUGUUGGUCU | 5840 | AGACCAACAGUCCCCGCCA |
| siRNA 351 | 351 | GGCGGGGACUGUUGGUCUC | 5841 | GAGACCAACAGUCCCCGCC |
| siRNA 352 | 352 | GCGGGGACUGUUGGUCUCG | 5842 | CGAGACCAACAGUCCCCGC |
| siRNA 353 | 353 | CGGGGACUGUUGGUCUCGG | 5843 | CCGAGACCAACAGUCCCCG |
| siRNA 354 | 354 | GGGGACUGUUGGUCUCGGC | 5844 | GCCGAGACCAACAGUCCCC |
| siRNA 355 | 355 | GGGACUGUUGGUCUCGGCC | 5845 | GGCCGAGACCAACAGUCCC |
| siRNA 356 | 356 | GGACUGUUGGUCUCGGCCC | 5846 | GGGCCGAGACCAACAGUCC |
| siRNA 357 | 357 | GACUGUUGGUCUCGGCCCU | 5847 | AGGGCCGAGACCAACAGUC |
| siRNA 358 | 358 | ACUGUUGGUCUCGGCCCUG | 5848 | CAGGGCCGAGACCAACAGU |
| siRNA 359 | 359 | CUGUUGGUCUCGGCCCUGC | 5849 | GCAGGGCCGAGACCAACAG |
| siRNA 360 | 360 | UGUUGGUCUCGGCCCUGCG | 5850 | CGCAGGGCCGAGACCAACA |
| siRNA 361 | 361 | GUUGGUCUCGGCCCUGCGG | 5851 | CCGCAGGGCCGAGACCAAC |
| siRNA 362 | 362 | UUGGUCUCGGCCCUGCGGA | 5852 | UCCGCAGGGCCGAGACCAA |
| siRNA 363 | 363 | UGGUCUCGGCCCUGCGGAA | 5853 | UUCCGCAGGGCCGAGACCA |
| siRNA 364 | 364 | GGUCUCGGCCCUGCGGAAG | 5854 | CUUCCGCAGGGCCGAGACC |
| siRNA 365 | 365 | GUCUCGGCCCUGCGGAAGG | 5855 | CCUUCCGCAGGGCCGAGAC |
| siRNA 366 | 366 | UCUCGGCCCUGCGGAAGGU | 5856 | ACCUUCCGCAGGGCCGAGA |
| siRNA 367 | 367 | CUCGGCCCUGCGGAAGGUU | 5857 | AACCUUCCGCAGGGCCGAG |
| siRNA 368 | 368 | UCGGCCCUGCGGAAGGUUC | 5858 | GAACCUUCCGCAGGGCCGA |
| siRNA 369 | 369 | CGGCCCUGCGGAAGGUUCA | 5859 | UGAACCUUCCGCAGGGCCG |
| siRNA 370 | 370 | GGCCCUGCGGAAGGUUCAG | 5860 | CUGAACCUUCCGCAGGGCC |
| siRNA 371 | 371 | GCCCUGCGGAAGGUUCAGG | 5861 | CCUGAACCUUCCGCAGGGC |
| siRNA 372 | 372 | CCCUGCGGAAGGUUCAGGA | 5862 | UCCUGAACCUUCCGCAGGG |
| siRNA 373 | 373 | CCUGCGGAAGGUUCAGGAA | 5863 | UUCCUGAACCUUCCGCAGG |
| siRNA 374 | 374 | CUGCGGAAGGUUCAGGAAC | 5864 | GUUCCUGAACCUUCCGCAG |
| siRNA 375 | 375 | UGCGGAAGGUUCAGGAACA | 5865 | UGUUCCUGAACCUUCCGCA |
| siRNA 376 | 376 | GCGGAAGGUUCAGGAACAA | 5866 | UUGUUCCUGAACCUUCCGC |
| siRNA 377 | 377 | CGGAAGGUUCAGGAACAAG | 5867 | CUUGUUCCUGAACCUUCCG |
| siRNA 378 | 378 | GGAAGGUUCAGGAACAAGU | 5868 | ACUUGUUCCUGAACCUUCC |
| siRNA 379 | 379 | GAAGGUUCAGGAACAAGUG | 5869 | CACUUGUUCCUGAACCUUC |
| siRNA 380 | 380 | AAGGUUCAGGAACAAGUGC | 5870 | GCACUUGUUCCUGAACCUU |
| siRNA 381 | 381 | AGGUUCAGGAACAAGUGCA | 5871 | UGCACUUGUUCCUGAACCU |
| siRNA 382 | 382 | GGUUCAGGAACAAGUGCAU | 5872 | AUGCACUUGUUCCUGAACC |
| siRNA 383 | 383 | GUUCAGGAACAAGUGCAUC | 5873 | GAUGCACUUGUUCCUGAAC |
| siRNA 384 | 384 | UUCAGGAACAAGUGCAUCC | 5874 | GGAUGCACUUGUUCCUGAA |
| siRNA 385 | 385 | UCAGGAACAAGUGCAUCCC | 5875 | GGGAUGCACUUGUUCCUGA |
| siRNA 386 | 386 | CAGGAACAAGUGCAUCCCA | 5876 | UGGGAUGCACUUGUUCCUG |
| siRNA 387 | 387 | AGGAACAAGUGCAUCCCAC | 5877 | GUGGGAUGCACUUGUUCCU |
| siRNA 388 | 388 | GGAACAAGUGCAUCCCACU | 5878 | AGUGGGAUGCACUUGUUCC |
| siRNA 389 | 389 | GAACAAGUGCAUCCCACUC | 5879 | GAGUGGGAUGCACUUGUUC |
| siRNA 390 | 390 | AACAAGUGCAUCCCACUCU | 5880 | AGAGUGGGAUGCACUUGUU |
| siRNA 391 | 391 | ACAAGUGCAUCCCACUCUC | 5881 | GAGAGUGGGAUGCACUUGU |
| siRNA 392 | 392 | CAAGUGCAUCCCACUCUCU | 5882 | AGAGAGUGGGAUGCACUUG |
| siRNA 393 | 393 | AAGUGCAUCCCACUCUCUC | 5883 | GAGAGAGUGGGAUGCACUU |
| siRNA 394 | 394 | AGUGCAUCCCACUCUCUCA | 5884 | UGAGAGAGUGGGAUGCACU |
| siRNA 395 | 395 | GUGCAUCCCACUCUCUCAG | 5885 | CUGAGAGAGUGGGAUGCAC |
| siRNA 396 | 396 | UGCAUCCCACUCUCUCAGC | 5886 | GCUGAGAGAGUGGGAUGCA |
| siRNA 397 | 397 | GCAUCCCACUCUCUCAGCU | 5887 | AGCUGAGAGAGUGGGAUGC |
| siRNA 398 | 398 | CAUCCCACUCUCUCAGCUA | 5888 | UAGCUGAGAGAGUGGGAUG |
| siRNA 399 | 399 | AUCCCACUCUCUCAGCUAA | 5889 | UUAGCUGAGAGAGUGGGAU |
| siRNA 400 | 400 | UCCCACUCUCUCAGCUAAU | 5890 | AUUAGCUGAGAGAGUGGGA |
| siRNA 401 | 401 | CCCACUCUCUCAGCUAAUG | 5891 | CAUUAGCUGAGAGAGUGGG |
| siRNA 402 | 402 | CCACUCUCUCAGCUAAUGA | 5892 | UCAUUAGCUGAGAGAGUGG |
| siRNA 403 | 403 | CACUCUCUCAGCUAAUGAA | 5893 | UUCAUUAGCUGAGAGAGUG |
| siRNA 404 | 404 | ACUCUCUCAGCUAAUGAAG | 5894 | CUUCAUUAGCUGAGAGAGU |
| siRNA 405 | 405 | CUCUCUCAGCUAAUGAAGA | 5895 | UCUUCAUUAGCUGAGAGAG |
| siRNA 406 | 406 | UCUCUCAGCUAAUGAAGAG | 5896 | CUCUUCAUUAGCUGAGAGA |
| siRNA 407 | 407 | CUCUCAGCUAAUGAAGAGU | 5897 | ACUCUUCAUUAGCUGAGAG |
| siRNA 408 | 408 | UCUCAGCUAAUGAAGAGUC | 5898 | GACUCUUCAUUAGCUGAGA |
| siRNA 409 | 409 | CUCAGCUAAUGAAGAGUCU | 5899 | AGACUCUUCAUUAGCUGAG |
| siRNA 410 | 410 | UCAGCUAAUGAAGAGUCUC | 5900 | GAGACUCUUCAUUAGCUGA |
| siRNA 411 | 411 | CAGCUAAUGAAGAGUCUCU | 5901 | AGAGACUCUUCAUUAGCUG |
| siRNA 412 | 412 | AGCUAAUGAAGAGUCUCUC | 5902 | GAGAGACUCUUCAUUAGCU |
| siRNA 413 | 413 | GCUAAUGAAGAGUCUCUCU | 5903 | AGAGAGACUCUUCAUUAGC |
| siRNA 414 | 414 | CUAAUGAAGAGUCUCUCUA | 5904 | UAGAGAGACUCUUCAUUAG |
| siRNA 415 | 415 | UAAUGAAGAGUCUCUCUAU | 5905 | AUAGAGAGACUCUUCAUUA |
| siRNA 416 | 416 | AAUGAAGAGUCUCUCUAUU | 5906 | AAUAGAGAGACUCUUCAUU |
| siRNA 417 | 417 | AUGAAGAGUCUCUCUAUUA | 5907 | UAAUAGAGAGACUCUUCAU |
| siRNA 418 | 418 | UGAAGAGUCUCUCUAUUAU | 5908 | AUAAUAGAGAGACUCUUCA |
| siRNA 419 | 419 | GAAGAGUCUCUCUAUUAUA | 5909 | UAUAAUAGAGAGACUCUUC |
| siRNA 420 | 420 | AAGAGUCUCUCUAUUAUAU | 5910 | AUAUAAUAGAGAGACUCUU |
| siRNA 421 | 421 | AGAGUCUCUCUAUUAUAUU | 5911 | AAUAUAAUAGAGAGACUCU |
| siRNA 422 | 422 | GAGUCUCUCUAUUAUAUUG | 5912 | CAAUAUAAUAGAGAGACUC |
| siRNA 423 | 423 | AGUCUCUCUAUUAUAUUGA | 5913 | UCAAUAUAAUAGAGAGACU |
| siRNA 424 | 424 | GUCUCUCUAUUAUAUUGAA | 5914 | UUCAAUAUAAUAGAGAGAC |
| siRNA 425 | 425 | UCUCUCUAUUAUAUUGAAG | 5915 | CUUCAAUAUAAUAGAGAGA |
| siRNA 426 | 426 | CUCUCUAUUAUAUUGAAGA | 5916 | UCUUCAAUAUAAUAGAGAG |
| siRNA 427 | 427 | UCUCUAUUAUAUUGAAGAG | 5917 | CUCUUCAAUAUAAUAGAGA |
| siRNA 428 | 428 | CUCUAUUAUAUUGAAGAGC | 5918 | GCUCUUCAAUAUAAUAGAG |
| siRNA 429 | 429 | UCUAUUAUAUUGAAGAGCU | 5919 | AGCUCUUCAAUAUAAUAGA |
| siRNA 430 | 430 | CUAUUAUAUUGAAGAGCUG | 5920 | CAGCUCUUCAAUAUAAUAG |
| siRNA 431 | 431 | UAUUAUAUUGAAGAGCUGA | 5921 | UCAGCUCUUCAAUAUAAUA |
| siRNA 432 | 432 | AUUAUAUUGAAGAGCUGAU | 5922 | AUCAGCUCUUCAAUAUAAU |
| siRNA 433 | 433 | UUAUAUUGAAGAGCUGAUU | 5923 | AAUCAGCUCUUCAAUAUAA |
| siRNA 434 | 434 | UAUAUUGAAGAGCUGAUUU | 5924 | AAAUCAGCUCUUCAAUAUA |
| siRNA 435 | 435 | AUAUUGAAGAGCUGAUUUU | 5925 | AAAAUCAGCUCUUCAAUAU |
| siRNA 436 | 436 | UAUUGAAGAGCUGAUUUUU | 5926 | AAAAAUCAGCUCUUCAAUA |
| siRNA 437 | 437 | AUUGAAGAGCUGAUUUUUC | 5927 | GAAAAAUCAGCUCUUCAAU |
| siRNA 438 | 438 | UUGAAGAGCUGAUUUUUCA | 5928 | UGAAAAAUCAGCUCUUCAA |
| siRNA 439 | 439 | UGAAGAGCUGAUUUUUCAG | 5929 | CUGAAAAAUCAGCUCUUCA |
| siRNA 440 | 440 | GAAGAGCUGAUUUUUCAGC | 5930 | GCUGAAAAAUCAGCUCUUC |
| siRNA 441 | 441 | AAGAGCUGAUUUUUCAGCU | 5931 | AGCUGAAAAAUCAGCUCUU |
| siRNA 442 | 442 | AGAGCUGAUUUUUCAGCUG | 5932 | CAGCUGAAAAAUCAGCUCU |
| siRNA 443 | 443 | GAGCUGAUUUUUCAGCUGC | 5933 | GCAGCUGAAAAAUCAGCUC |
| siRNA 444 | 444 | AGCUGAUUUUUCAGCUGCU | 5934 | AGCAGCUGAAAAAUCAGCU |
| siRNA 445 | 445 | GCUGAUUUUUCAGCUGCUU | 5935 | AAGCAGCUGAAAAAUCAGC |
| siRNA 446 | 446 | CUGAUUUUUCAGCUGCUUA | 5936 | UAAGCAGCUGAAAAAUCAG |
| siRNA 447 | 447 | UGAUUUUUCAGCUGCUUAA | 5937 | UUAAGCAGCUGAAAAAUCA |
| siRNA 448 | 448 | GAUUUUUCAGCUGCUUAAU | 5938 | AUUAAGCAGCUGAAAAAUC |
| siRNA 449 | 449 | AUUUUUCAGCUGCUUAAUA | 5939 | UAUUAAGCAGCUGAAAAAU |
| siRNA 450 | 450 | UUUUUCAGCUGCUUAAUAA | 5940 | UUAUUAAGCAGCUGAAAAA |
| siRNA 451 | 451 | UUUUCAGCUGCUUAAUAAA | 5941 | UUUAUUAAGCAGCUGAAAA |
| siRNA 452 | 452 | UUUCAGCUGCUUAAUAAAU | 5942 | AUUUAUUAAGCAGCUGAAA |
| siRNA 453 | 453 | UUCAGCUGCUUAAUAAAUU | 5943 | AAUUUAUUAAGCAGCUGAA |
| siRNA 454 | 454 | UCAGCUGCUUAAUAAAUUA | 5944 | UAAUUUAUUAAGCAGCUGA |
| siRNA 455 | 455 | CAGCUGCUUAAUAAAUUAU | 5945 | AUAAUUUAUUAAGCAGCUG |
| siRNA 456 | 456 | AGCUGCUUAAUAAAUUAUG | 5946 | CAUAAUUUAUUAAGCAGCU |
| siRNA 457 | 457 | GCUGCUUAAUAAAUUAUGC | 5947 | GCAUAAUUUAUUAAGCAGC |
| siRNA 458 | 458 | CUGCUUAAUAAAUUAUGCA | 5948 | UGCAUAAUUUAUUAAGCAG |
| siRNA 459 | 459 | UGCUUAAUAAAUUAUGCAU | 5949 | AUGCAUAAUUUAUUAAGCA |
| siRNA 460 | 460 | GCUUAAUAAAUUAUGCAUG | 5950 | CAUGCAUAAUUUAUUAAGC |
| siRNA 461 | 461 | CUUAAUAAAUUAUGCAUGG | 5951 | CCAUGCAUAAUUUAUUAAG |
| siRNA 462 | 462 | UUAAUAAAUUAUGCAUGGC | 5952 | GCCAUGCAUAAUUUAUUAA |
| siRNA 463 | 463 | UAAUAAAUUAUGCAUGGCC | 5953 | GGCCAUGCAUAAUUUAUUA |
| siRNA 464 | 464 | AAUAAAUUAUGCAUGGCCC | 5954 | GGGCCAUGCAUAAUUUAUU |
| siRNA 465 | 465 | AUAAAUUAUGCAUGGCCCA | 5955 | UGGGCCAUGCAUAAUUUAU |
| siRNA 466 | 466 | UAAAUUAUGCAUGGCCCAG | 5956 | CUGGGCCAUGCAUAAUUUA |
| siRNA 467 | 467 | AAAUUAUGCAUGGCCCAGC | 5957 | GCUGGGCCAUGCAUAAUUU |
| siRNA 468 | 468 | AAUUAUGCAUGGCCCAGCC | 5958 | GGCUGGGCCAUGCAUAAUU |
| siRNA 469 | 469 | AUUAUGCAUGGCCCAGCCA | 5959 | UGGCUGGGCCAUGCAUAAU |
| siRNA 470 | 470 | UUAUGCAUGGCCCAGCCAA | 5960 | UUGGCUGGGCCAUGCAUAA |
| siRNA 471 | 471 | UAUGCAUGGCCCAGCCAAG | 5961 | CUUGGCUGGGCCAUGCAUA |
| siRNA 472 | 472 | AUGCAUGGCCCAGCCAAGG | 5962 | CCUUGGCUGGGCCAUGCAU |
| siRNA 473 | 473 | UGCAUGGCCCAGCCAAGGA | 5963 | UCCUUGGCUGGGCCAUGCA |
| siRNA 474 | 474 | GCAUGGCCCAGCCAAGGAC | 5964 | GUCCUUGGCUGGGCCAUGC |
| siRNA 475 | 475 | CAUGGCCCAGCCAAGGACU | 5965 | AGUCCUUGGCUGGGCCAUG |
| siRNA 476 | 476 | AUGGCCCAGCCAAGGACUG | 5966 | CAGUCCUUGGCUGGGCCAU |
| siRNA 477 | 477 | UGGCCCAGCCAAGGACUGU | 5967 | ACAGUCCUUGGCUGGGCCA |
| siRNA 478 | 478 | GGCCCAGCCAAGGACUGUU | 5968 | AACAGUCCUUGGCUGGGCC |
| siRNA 479 | 479 | GCCCAGCCAAGGACUGUUC | 5969 | GAACAGUCCUUGGCUGGGC |
| siRNA 480 | 480 | CCCAGCCAAGGACUGUUCA | 5970 | UGAACAGUCCUUGGCUGGG |
| siRNA 481 | 481 | CCAGCCAAGGACUGUUCAA | 5971 | UUGAACAGUCCUUGGCUGG |
| siRNA 482 | 482 | CAGCCAAGGACUGUUCAAG | 5972 | CUUGAACAGUCCUUGGCUG |
| siRNA 483 | 483 | AGCCAAGGACUGUUCAAGA | 5973 | UCUUGAACAGUCCUUGGCU |
| siRNA 484 | 484 | GCCAAGGACUGUUCAAGAU | 5974 | AUCUUGAACAGUCCUUGGC |
| siRNA 485 | 485 | CCAAGGACUGUUCAAGAUG | 5975 | CAUCUUGAACAGUCCUUGG |
| siRNA 486 | 486 | CAAGGACUGUUCAAGAUGU | 5976 | ACAUCUUGAACAGUCCUUG |
| siRNA 487 | 487 | AAGGACUGUUCAAGAUGUA | 5977 | UACAUCUUGAACAGUCCUU |
| siRNA 488 | 488 | AGGACUGUUCAAGAUGUAG | 5978 | CUACAUCUUGAACAGUCCU |
| siRNA 489 | 489 | GGACUGUUCAAGAUGUAGA | 5979 | UCUACAUCUUGAACAGUCC |
| siRNA 490 | 490 | GACUGUUCAAGAUGUAGAG | 5980 | CUCUACAUCUUGAACAGUC |
| siRNA 491 | 491 | ACUGUUCAAGAUGUAGAGG | 5981 | CCUCUACAUCUUGAACAGU |
| siRNA 492 | 492 | CUGUUCAAGAUGUAGAGGA | 5982 | UCCUCUACAUCUUGAACAG |
| siRNA 493 | 493 | UGUUCAAGAUGUAGAGGAG | 5983 | CUCCUCUACAUCUUGAACA |
| siRNA 494 | 494 | GUUCAAGAUGUAGAGGAGC | 5984 | GCUCCUCUACAUCUUGAAC |
| siRNA 495 | 495 | UUCAAGAUGUAGAGGAGCG | 5985 | CGCUCCUCUACAUCUUGAA |
| siRNA 496 | 496 | UCAAGAUGUAGAGGAGCGA | 5986 | UCGCUCCUCUACAUCUUGA |
| siRNA 497 | 497 | CAAGAUGUAGAGGAGCGAG | 5987 | CUCGCUCCUCUACAUCUUG |
| siRNA 498 | 498 | AAGAUGUAGAGGAGCGAGU | 5988 | ACUCGCUCCUCUACAUCUU |
| siRNA 499 | 499 | AGAUGUAGAGGAGCGAGUU | 5989 | AACUCGCUCCUCUACAUCU |
| siRNA 500 | 500 | GAUGUAGAGGAGCGAGUUC | 5990 | GAACUCGCUCCUCUACAUC |
| siRNA 501 | 501 | AUGUAGAGGAGCGAGUUCA | 5991 | UGAACUCGCUCCUCUACAU |
| siRNA 502 | 502 | UGUAGAGGAGCGAGUUCAG | 5992 | CUGAACUCGCUCCUCUACA |
| siRNA 503 | 503 | GUAGAGGAGCGAGUUCAGA | 5993 | UCUGAACUCGCUCCUCUAC |
| siRNA 504 | 504 | UAGAGGAGCGAGUUCAGAA | 5994 | UUCUGAACUCGCUCCUCUA |
| siRNA 505 | 505 | AGAGGAGCGAGUUCAGAAG | 5995 | CUUCUGAACUCGCUCCUCU |
| siRNA 506 | 506 | GAGGAGCGAGUUCAGAAGA | 5996 | UCUUCUGAACUCGCUCCUC |
| siRNA 507 | 507 | AGGAGCGAGUUCAGAAGAC | 5997 | GUCUUCUGAACUCGCUCCU |
| siRNA 508 | 508 | GGAGCGAGUUCAGAAGACC | 5998 | GGUCUUCUGAACUCGCUCC |
| siRNA 509 | 509 | GAGCGAGUUCAGAAGACCU | 5999 | AGGUCUUCUGAACUCGCUC |
| siRNA 510 | 510 | AGCGAGUUCAGAAGACCUU | 6000 | AAGGUCUUCUGAACUCGCU |
| siRNA 511 | 511 | GCGAGUUCAGAAGACCUUU | 6001 | AAAGGUCUUCUGAACUCGC |
| siRNA 512 | 512 | CGAGUUCAGAAGACCUUUC | 6002 | GAAAGGUCUUCUGAACUCG |
| siRNA 513 | 513 | GAGUUCAGAAGACCUUUCC | 6003 | GGAAAGGUCUUCUGAACUC |
| siRNA 514 | 514 | AGUUCAGAAGACCUUUCCU | 6004 | AGGAAAGGUCUUCUGAACU |
| siRNA 515 | 515 | GUUCAGAAGACCUUUCCUC | 6005 | GAGGAAAGGUCUUCUGAAC |
| siRNA 516 | 516 | UUCAGAAGACCUUUCCUCA | 6006 | UGAGGAAAGGUCUUCUGAA |
| siRNA 517 | 517 | UCAGAAGACCUUUCCUCAC | 6007 | GUGAGGAAAGGUCUUCUGA |
| siRNA 518 | 518 | CAGAAGACCUUUCCUCACC | 6008 | GGUGAGGAAAGGUCUUCUG |
| siRNA 519 | 519 | AGAAGACCUUUCCUCACCC | 6009 | GGGUGAGGAAAGGUCUUCU |
| siRNA 520 | 520 | GAAGACCUUUCCUCACCCA | 6010 | UGGGUGAGGAAAGGUCUUC |
| siRNA 521 | 521 | AAGACCUUUCCUCACCCAA | 6011 | UUGGGUGAGGAAAGGUCUU |
| siRNA 522 | 522 | AGACCUUUCCUCACCCAAU | 6012 | AUUGGGUGAGGAAAGGUCU |
| siRNA 523 | 523 | GACCUUUCCUCACCCAAUU | 6013 | AAUUGGGUGAGGAAAGGUC |
| siRNA 524 | 524 | ACCUUUCCUCACCCAAUUG | 6014 | CAAUUGGGUGAGGAAAGGU |
| siRNA 525 | 525 | CCUUUCCUCACCCAAUUGA | 6015 | UCAAUUGGGUGAGGAAAGG |
| siRNA 526 | 526 | CUUUCCUCACCCAAUUGAU | 6016 | AUCAAUUGGGUGAGGAAAG |
| siRNA 527 | 527 | UUUCCUCACCCAAUUGAUA | 6017 | UAUCAAUUGGGUGAGGAAA |
| siRNA 528 | 528 | UUCCUCACCCAAUUGAUAA | 6018 | UUAUCAAUUGGGUGAGGAA |
| siRNA 529 | 529 | UCCUCACCCAAUUGAUAAA | 6019 | UUUAUCAAUUGGGUGAGGA |
| siRNA 530 | 530 | CCUCACCCAAUUGAUAAAU | 6020 | AUUUAUCAAUUGGGUGAGG |
| siRNA 531 | 531 | CUCACCCAAUUGAUAAAUG | 6021 | CAUUUAUCAAUUGGGUGAG |
| siRNA 532 | 532 | UCACCCAAUUGAUAAAUGG | 6022 | CCAUUUAUCAAUUGGGUGA |
| siRNA 533 | 533 | CACCCAAUUGAUAAAUGGG | 6023 | CCCAUUUAUCAAUUGGGUG |
| siRNA 534 | 534 | ACCCAAUUGAUAAAUGGGC | 6024 | GCCCAUUUAUCAAUUGGGU |
| siRNA 535 | 535 | CCCAAUUGAUAAAUGGGCC | 6025 | GGCCCAUUUAUCAAUUGGG |
| siRNA 536 | 536 | CCAAUUGAUAAAUGGGCCA | 6026 | UGGCCCAUUUAUCAAUUGG |
| siRNA 537 | 537 | CAAUUGAUAAAUGGGCCAU | 6027 | AUGGCCCAUUUAUCAAUUG |
| siRNA 538 | 538 | AAUUGAUAAAUGGGCCAUU | 6028 | AAUGGCCCAUUUAUCAAUU |
| siRNA 539 | 539 | AUUGAUAAAUGGGCCAUUG | 6029 | CAAUGGCCCAUUUAUCAAU |
| siRNA 540 | 540 | UUGAUAAAUGGGCCAUUGC | 6030 | GCAAUGGCCCAUUUAUCAA |
| siRNA 541 | 541 | UGAUAAAUGGGCCAUUGCU | 6031 | AGCAAUGGCCCAUUUAUCA |
| siRNA 542 | 542 | GAUAAAUGGGCCAUUGCUG | 6032 | CAGCAAUGGCCCAUUUAUC |
| siRNA 543 | 543 | AUAAAUGGGCCAUUGCUGA | 6033 | UCAGCAAUGGCCCAUUUAU |
| siRNA 544 | 544 | UAAAUGGGCCAUUGCUGAU | 6034 | AUCAGCAAUGGCCCAUUUA |
| siRNA 545 | 545 | AAAUGGGCCAUUGCUGAUG | 6035 | CAUCAGCAAUGGCCCAUUU |
| siRNA 546 | 546 | AAUGGGCCAUUGCUGAUGC | 6036 | GCAUCAGCAAUGGCCCAUU |
| siRNA 547 | 547 | AUGGGCCAUUGCUGAUGCA | 6037 | UGCAUCAGCAAUGGCCCAU |
| siRNA 548 | 548 | UGGGCCAUUGCUGAUGCAC | 6038 | GUGCAUCAGCAAUGGCCCA |
| siRNA 549 | 549 | GGGCCAUUGCUGAUGCACA | 6039 | UGUGCAUCAGCAAUGGCCC |
| siRNA 550 | 550 | GGCCAUUGCUGAUGCACAA | 6040 | UUGUGCAUCAGCAAUGGCC |
| siRNA 551 | 551 | GCCAUUGCUGAUGCACAAU | 6041 | AUUGUGCAUCAGCAAUGGC |
| siRNA 552 | 552 | CCAUUGCUGAUGCACAAUC | 6042 | GAUUGUGCAUCAGCAAUGG |
| siRNA 553 | 553 | CAUUGCUGAUGCACAAUCU | 6043 | AGAUUGUGCAUCAGCAAUG |
| siRNA 554 | 554 | AUUGCUGAUGCACAAUCUG | 6044 | CAGAUUGUGCAUCAGCAAU |
| siRNA 555 | 555 | UUGCUGAUGCACAAUCUGC | 6045 | GCAGAUUGUGCAUCAGCAA |
| siRNA 556 | 556 | UGCUGAUGCACAAUCUGCU | 6046 | AGCAGAUUGUGCAUCAGCA |
| siRNA 557 | 557 | GCUGAUGCACAAUCUGCUA | 6047 | UAGCAGAUUGUGCAUCAGC |
| siRNA 558 | 558 | CUGAUGCACAAUCUGCUAU | 6048 | AUAGCAGAUUGUGCAUCAG |
| siRNA 559 | 559 | UGAUGCACAAUCUGCUAUA | 6049 | UAUAGCAGAUUGUGCAUCA |
| siRNA 560 | 560 | GAUGCACAAUCUGCUAUAG | 6050 | CUAUAGCAGAUUGUGCAUC |
| siRNA 561 | 561 | AUGCACAAUCUGCUAUAGA | 6051 | UCUAUAGCAGAUUGUGCAU |
| siRNA 562 | 562 | UGCACAAUCUGCUAUAGAA | 6052 | UUCUAUAGCAGAUUGUGCA |
| siRNA 563 | 563 | GCACAAUCUGCUAUAGAAA | 6053 | UUUCUAUAGCAGAUUGUGC |
| siRNA 564 | 564 | CACAAUCUGCUAUAGAAAA | 6054 | UUUUCUAUAGCAGAUUGUG |
| siRNA 565 | 565 | ACAAUCUGCUAUAGAAAAA | 6055 | UUUUUCUAUAGCAGAUUGU |
| siRNA 566 | 566 | CAAUCUGCUAUAGAAAAAC | 6056 | GUUUUUCUAUAGCAGAUUG |
| siRNA 567 | 567 | AAUCUGCUAUAGAAAAACG | 6057 | CGUUUUUCUAUAGCAGAUU |
| siRNA 568 | 568 | AUCUGCUAUAGAAAAACGA | 6058 | UCGUUUUUCUAUAGCAGAU |
| siRNA 569 | 569 | UCUGCUAUAGAAAAACGAA | 6059 | UUCGUUUUUCUAUAGCAGA |
| siRNA 570 | 570 | CUGCUAUAGAAAAACGAAA | 6060 | UUUCGUUUUUCUAUAGCAG |
| siRNA 571 | 571 | UGCUAUAGAAAAACGAAAA | 6061 | UUUUCGUUUUUCUAUAGCA |
| siRNA 572 | 572 | GCUAUAGAAAAACGAAAAC | 6062 | GUUUUCGUUUUUCUAUAGC |
| siRNA 573 | 573 | CUAUAGAAAAACGAAAACG | 6063 | CGUUUUCGUUUUUCUAUAG |
| siRNA 574 | 574 | UAUAGAAAAACGAAAACGA | 6064 | UCGUUUUCGUUUUUCUAUA |
| siRNA 575 | 575 | AUAGAAAAACGAAAACGAA | 6065 | UUCGUUUUCGUUUUUCUAU |
| siRNA 576 | 576 | UAGAAAAACGAAAACGAAG | 6066 | CUUCGUUUUCGUUUUUCUA |
| siRNA 577 | 577 | AGAAAAACGAAAACGAAGA | 6067 | UCUUCGUUUUCGUUUUUCU |
| siRNA 578 | 578 | GAAAAACGAAAACGAAGAA | 6068 | UUCUUCGUUUUCGUUUUUC |
| siRNA 579 | 579 | AAAAACGAAAACGAAGAAA | 6069 | UUUCUUCGUUUUCGUUUUU |
| siRNA 580 | 580 | AAAACGAAAACGAAGAAAU | 6070 | AUUUCUUCGUUUUCGUUUU |
| siRNA 581 | 581 | AAACGAAAACGAAGAAAUC | 6071 | GAUUUCUUCGUUUUCGUUU |
| siRNA 582 | 582 | AACGAAAACGAAGAAAUCC | 6072 | GGAUUUCUUCGUUUUCGUU |
| siRNA 583 | 583 | ACGAAAACGAAGAAAUCCU | 5073 | AGGAUUUCUUCGUUUUCGU |
| siRNA 584 | 584 | CGAAAACGAAGAAAUCCUC | 6074 | GAGGAUUUCUUCGUUUUCG |
| siRNA 585 | 585 | GAAAACGAAGAAAUCCUCU | 6075 | AGAGGAUUUCUUCGUUUUC |
| siRNA 586 | 586 | AAAACGAAGAAAUCCUCUU | 6076 | AAGAGGAUUUCUUCGUUUU |
| siRNA 587 | 587 | AAACGAAGAAAUCCUCUUU | 6077 | AAAGAGGAUUUCUUCGUUU |
| siRNA 588 | 588 | AACGAAGAAAUCCUCUUUU | 6078 | AAAAGAGGAUUUCUUCGUU |
| siRNA 589 | 589 | ACGAAGAAAUCCUCUUUUA | 6079 | UAAAAGAGGAUUUCUUCGU |
| siRNA 590 | 590 | CGAAGAAAUCCUCUUUUAC | 6080 | GUAAAAGAGGAUUUCUUCG |
| siRNA 591 | 591 | GAAGAAAUCCUCUUUUACU | 6081 | AGUAAAAGAGGAUUUCUUC |
| siRNA 592 | 592 | AAGAAAUCCUCUUUUACUG | 6082 | CAGUAAAAGAGGAUUUCUU |
| siRNA 593 | 593 | AGAAAUCCUCUUUUACUGC | 6083 | GCAGUAAAAGAGGAUUUCU |
| siRNA 594 | 594 | GAAAUCCUCUUUUACUGCC | 6084 | GGCAGUAAAAGAGGAUUUC |
| siRNA 595 | 595 | AAAUCCUCUUUUACUGCCU | 6085 | AGGCAGUAAAAGAGGAUUU |
| siRNA 596 | 596 | AAUCCUCUUUUACUGCCUG | 6086 | CAGGCAGUAAAAGAGGAUU |
| siRNA 597 | 597 | AUCCUCUUUUACUGCCUGU | 6087 | ACAGGCAGUAAAAGAGGAU |
| siRNA 598 | 598 | UCCUCUUUUACUGCCUGUG | 6088 | CACAGGCAGUAAAAGAGGA |
| siRNA 599 | 599 | CCUCUUUUACUGCCUGUGG | 6089 | CCACAGGCAGUAAAAGAGG |
| siRNA 600 | 600 | CUCUUUUACUGCCUGUGGA | 6090 | UCCACAGGCAGUAAAAGAG |
| siRNA 601 | 601 | UCUUUUACUGCCUGUGGAC | 6091 | GUCCACAGGCAGUAAAAGA |
| siRNA 602 | 602 | CUUUUACUGCCUGUGGACA | 6092 | UGUCCACAGGCAGUAAAAG |
| siRNA 603 | 603 | UUUUACUGCCUGUGGACAA | 6093 | UUGUCCACAGGCAGUAAAA |
| siRNA 604 | 604 | UUUACUGCCUGUGGACAAA | 6094 | UUUGUCCACAGGCAGUAAA |
| siRNA 605 | 605 | UUACUGCCUGUGGACAAAA | 6095 | UUUUGUCCACAGGCAGUAA |
| siRNA 606 | 606 | UACUGCCUGUGGACAAAAU | 6096 | AUUUUGUCCACAGGCAGUA |
| siRNA 607 | 607 | ACUGCCUGUGGACAAAAUC | 6097 | GAUUUUGUCCACAGGCAGU |
| siRNA 608 | 608 | CUGCCUGUGGACAAAAUCC | 6098 | GGAUUUUGUCCACAGGCAG |
| siRNA 609 | 609 | UGCCUGUGGACAAAAUCCA | 6099 | UGGAUUUUGUCCACAGGCA |
| siRNA 610 | 610 | GCCUGUGGACAAAAUCCAU | 6100 | AUGGAUUUUGUCCACAGGC |
| siRNA 611 | 611 | CCUGUGGACAAAAUCCAUC | 6101 | GAUGGAUUUUGUCCACAGG |
| siRNA 612 | 612 | CUGUGGACAAAAUCCAUCC | 6102 | GGAUGGAUUUUGUCCACAG |
| siRNA 613 | 613 | UGUGGACAAAAUCCAUCCU | 6103 | AGGAUGGAUUUUGUCCACA |
| siRNA 614 | 614 | GUGGACAAAAUCCAUCCUU | 6104 | AAGGAUGGAUUUUGUCCAC |
| siRNA 615 | 615 | UGGACAAAAUCCAUCCUUC | 6105 | GAAGGAUGGAUUUUGUCCA |
| siRNA 616 | 616 | GGACAAAAUCCAUCCUUCG | 6106 | CGAAGGAUGGAUUUUGUCC |
| siRNA 617 | 617 | GACAAAAUCCAUCCUUCGU | 6107 | ACGAAGGAUGGAUUUUGUC |
| siRNA 618 | 618 | ACAAAAUCCAUCCUUCGUU | 6108 | AACGAAGGAUGGAUUUUGU |
| siRNA 619 | 619 | CAAAAUCCAUCCUUCGUUG | 6109 | CAACGAAGGAUGGAUUUUG |
| siRNA 620 | 620 | AAAAUCCAUCCUUCGUUGA | 6110 | UCAACGAAGGAUGGAUUUU |
| siRNA 621 | 621 | AAAUCCAUCCUUCGUUGAA | 6111 | UUCAACGAAGGAUGGAUUU |
| siRNA 622 | 622 | AAUCCAUCCUUCGUUGAAG | 6112 | CUUCAACGAAGGAUGGAUU |
| siRNA 623 | 623 | AUCCAUCCUUCGUUGAAGG | 6113 | CCUUCAACGAAGGAUGGAU |
| siRNA 624 | 624 | UCCAUCCUUCGUUGAAGGA | 6114 | UCCUUCAACGAAGGAUGGA |
| siRNA 625 | 625 | CCAUCCUUCGUUGAAGGAA | 6115 | UUCCUUCAACGAAGGAUGG |
| siRNA 626 | 626 | CAUCCUUCGUUGAAGGAAG | 6116 | CUUCCUUCAACGAAGGAUG |
| siRNA 627 | 627 | AUCCUUCGUUGAAGGAAGU | 6117 | ACUUCCUUCAACGAAGGAU |
| siRNA 628 | 628 | UCCUUCGUUGAAGGAAGUA | 6118 | UACUUCCUUCAACGAAGGA |
| siRNA 629 | 629 | CCUUCGUUGAAGGAAGUAU | 6119 | AUACUUCCUUCAACGAAGG |
| siRNA 630 | 630 | CUUCGUUGAAGGAAGUAUU | 6120 | AAUACUUCCUUCAACGAAG |
| siRNA 631 | 631 | UUCGUUGAAGGAAGUAUUA | 6121 | UAAUACUUCCUUCAACGAA |
| siRNA 632 | 632 | UCGUUGAAGGAAGUAUUAG | 6122 | CUAAUACUUCCUUCAACGA |
| siRNA 633 | 633 | CGUUGAAGGAAGUAUUAGG | 6123 | CCUAAUACUUCCUUCAACG |
| siRNA 634 | 634 | GUUGAAGGAAGUAUUAGGG | 6124 | CCCUAAUACUUCCUUCAAC |
| siRNA 635 | 635 | UUGAAGGAAGUAUUAGGGU | 6125 | ACCCUAAUACUUCCUUCAA |
| siRNA 636 | 636 | UGAAGGAAGUAUUAGGGUA | 6126 | UACCCUAAUACUUCCUUCA |
| siRNA 637 | 637 | GAAGGAAGUAUUAGGGUAC | 6127 | GUACCCUAAUACUUCCUUC |
| siRNA 638 | 638 | AAGGAAGUAUUAGGGUACA | 6128 | UGUACCCUAAUACUUCCUU |
| siRNA 639 | 639 | AGGAAGUAUUAGGGUACAA | 6129 | UUGUACCCUAAUACUUCCU |
| siRNA 640 | 640 | GGAAGUAUUAGGGUACAAA | 6130 | UUUGUACCCUAAUACUUCC |
| siRNA 641 | 641 | GAAGUAUUAGGGUACAAAG | 6131 | CUUUGUACCCUAAUACUUC |
| siRNA 642 | 642 | AAGUAUUAGGGUACAAAGU | 6132 | ACUUUGUACCCUAAUACUU |
| siRNA 643 | 643 | AGUAUUAGGGUACAAAGUG | 6133 | CACUUUGUACCCUAAUACU |
| siRNA 644 | 644 | GUAUUAGGGUACAAAGUGG | 6134 | CCACUUUGUACCCUAAUAC |
| siRNA 645 | 645 | UAUUAGGGUACAAAGUGGA | 6135 | UCCACUUUGUACCCUAAUA |
| siRNA 646 | 646 | AUUAGGGUACAAAGUGGAC | 6136 | GUCCACUUUGUACCCUAAU |
| siRNA 647 | 647 | UUAGGGUACAAAGUGGACU | 6137 | AGUCCACUUUGUACCCUAA |
| siRNA 648 | 648 | UAGGGUACAAAGUGGACUA | 6138 | UAGUCCACUUUGUACCCUA |
| siRNA 649 | 649 | AGGGUACAAAGUGGACUAC | 6139 | GUAGUCCACUUUGUACCCU |
| siRNA 650 | 650 | GGGUACAAAGUGGACUACC | 6140 | GGUAGUCCACUUUGUACCC |
| siRNA 651 | 651 | GGUACAAAGUGGACUACCA | 6141 | UGGUAGUCCACUUUGUACC |
| siRNA 652 | 652 | GUACAAAGUGGACUACCAU | 6142 | AUGGUAGUCCACUUUGUAC |
| siRNA 653 | 653 | UACAAAGUGGACUACCAUG | 6143 | CAUGGUAGUCCACUUUGUA |
| siRNA 654 | 654 | ACAAAGUGGACUACCAUGU | 6144 | ACAUGGUAGUCCACUUUGU |
| siRNA 655 | 655 | CAAAGUGGACUACCAUGUA | 6145 | UACAUGGUAGUCCACUUUG |
| siRNA 656 | 656 | AAAGUGGACUACCAUGUAU | 6146 | AUACAUGGUAGUCCACUUU |
| siRNA 657 | 657 | AAGUGGACUACCAUGUAUC | 6147 | GAUACAUGGUAGUCCACUU |
| siRNA 658 | 658 | AGUGGACUACCAUGUAUCC | 6148 | GGAUACAUGGUAGUCCACU |
| siRNA 659 | 659 | GUGGACUACCAUGUAUCCC | 6149 | GGGAUACAUGGUAGUCCAC |
| siRNA 660 | 660 | UGGACUACCAUGUAUCCCU | 6150 | AGGGAUACAUGGUAGUCCA |
| siRNA 661 | 661 | GGACUACCAUGUAUCCCUA | 6151 | UAGGGAUACAUGGUAGUCC |
| siRNA 662 | 662 | GACUACCAUGUAUCCCUAU | 6152 | AUAGGGAUACAUGGUAGUC |
| siRNA 663 | 663 | ACUACCAUGUAUCCCUAUA | 6153 | UAUAGGGAUACAUGGUAGU |
| siRNA 664 | 664 | CUACCAUGUAUCCCUAUAU | 6154 | AUAUAGGGAUACAUGGUAG |
| siRNA 665 | 665 | UACCAUGUAUCCCUAUAUA | 6155 | UAUAUAGGGAUACAUGGUA |
| siRNA 666 | 666 | ACCAUGUAUCCCUAUAUAU | 6156 | AUAUAUAGGGAUACAUGGU |
| siRNA 667 | 667 | CCAUGUAUCCCUAUAUAUU | 6157 | AAUAUAUAGGGAUACAUGG |
| siRNA 668 | 668 | CAUGUAUCCCUAUAUAUUG | 6158 | CAAUAUAUAGGGAUACAUG |
| siRNA 669 | 669 | AUGUAUCCCUAUAUAUUGU | 6159 | ACAAUAUAUAGGGAUACAU |
| siRNA 670 | 670 | UGUAUCCCUAUAUAUUGUG | 6160 | CACAAUAUAUAGGGAUACA |
| siRNA 671 | 671 | GUAUCCCUAUAUAUUGUGG | 6161 | CCACAAUAUAUAGGGAUAC |
| siRNA 672 | 672 | UAUCCCUAUAUAUUGUGGC | 6162 | GCCACAAUAUAUAGGGAUA |
| siRNA 673 | 673 | AUCCCUAUAUAUUGUGGCU | 6163 | AGCCACAAUAUAUAGGGAU |
| siRNA 674 | 674 | UCCCUAUAUAUUGUGGCUG | 6164 | CAGCCACAAUAUAUAGGGA |
| siRNA 675 | 675 | CCCUAUAUAUUGUGGCUGU | 6165 | ACAGCCACAAUAUAUAGGG |
| siRNA 676 | 676 | CCUAUAUAUUGUGGCUGUA | 6166 | UACAGCCACAAUAUAUAGG |
| siRNA 677 | 677 | CUAUAUAUUGUGGCUGUAC | 6167 | GUACAGCCACAAUAUAUAG |
| siRNA 678 | 678 | UAUAUAUUGUGGCUGUACU | 6168 | AGUACAGCCACAAUAUAUA |
| siRNA 679 | 679 | AUAUAUUGUGGCUGUACUA | 6169 | UAGUACAGCCACAAUAUAU |
| siRNA 680 | 680 | UAUAUUGUGGCUGUACUAG | 6170 | CUAGUACAGCCACAAUAUA |
| siRNA 681 | 681 | AUAUUGUGGCUGUACUAGA | 6171 | UCUAGUACAGCCACAAUAU |
| siRNA 682 | 682 | UAUUGUGGCUGUACUAGAG | 6172 | CUCUAGUACAGCCACAAUA |
| siRNA 683 | 683 | AUUGUGGCUGUACUAGAGU | 6173 | ACUCUAGUACAGCCACAAU |
| siRNA 684 | 684 | UUGUGGCUGUACUAGAGUA | 6174 | UACUCUAGUACAGCCACAA |
| siRNA 685 | 685 | UGUGGCUGUACUAGAGUAU | 6175 | AUACUCUAGUACAGCCACA |
| siRNA 686 | 686 | GUGGCUGUACUAGAGUAUA | 6176 | UAUACUCUAGUACAGCCAC |
| siRNA 687 | 687 | UGGCUGUACUAGAGUAUAU | 6177 | AUAUACUCUAGUACAGCCA |
| siRNA 688 | 688 | GGCUGUACUAGAGUAUAUC | 6178 | GAUAUACUCUAGUACAGCC |
| siRNA 689 | 689 | GCUGUACUAGAGUAUAUCU | 6179 | AGAUAUACUCUAGUACAGC |
| siRNA 690 | 690 | CUGUACUAGAGUAUAUCUC | 6180 | GAGAUAUACUCUAGUACAG |
| siRNA 691 | 691 | UGUACUAGAGUAUAUCUCA | 6181 | UGAGAUAUACUCUAGUACA |
| siRNA 692 | 692 | GUACUAGAGUAUAUCUCAG | 6182 | CUGAGAUAUACUCUAGUAC |
| siRNA 693 | 693 | UACUAGAGUAUAUCUCAGC | 6183 | GCUGAGAUAUACUCUAGUA |
| siRNA 694 | 694 | ACUAGAGUAUAUCUCAGCU | 6184 | AGCUGAGAUAUACUCUAGU |
| siRNA 695 | 695 | CUAGAGUAUAUCUCAGCUG | 6185 | CAGCUGAGAUAUACUCUAG |
| siRNA 696 | 696 | UAGAGUAUAUCUCAGCUGA | 6186 | UCAGCUGAGAUAUACUCUA |
| siRNA 697 | 697 | AGAGUAUAUCUCAGCUGAU | 6187 | AUCAGCUGAGAUAUACUCU |
| siRNA 698 | 698 | GAGUAUAUCUCAGCUGAUA | 6188 | UAUCAGCUGAGAUAUACUC |
| siRNA 699 | 699 | AGUAUAUCUCAGCUGAUAU | 6189 | AUAUCAGCUGAGAUAUACU |
| siRNA 700 | 700 | GUAUAUCUCAGCUGAUAUU | 6190 | AAUAUCAGCUGAGAUAUAC |
| siRNA 701 | 701 | UAUAUCUCAGCUGAUAUUU | 6191 | AAAUAUCAGCUGAGAUAUA |
| siRNA 702 | 702 | AUAUCUCAGCUGAUAUUUU | 6192 | AAAAUAUCAGCUGAGAUAU |
| siRNA 703 | 703 | UAUCUCAGCUGAUAUUUUA | 6193 | UAAAAUAUCAGCUGAGAUA |
| siRNA 704 | 704 | AUCUCAGCUGAUAUUUUAA | 6194 | UUAAAAUAUCAGCUGAGAU |
| siRNA 705 | 705 | UCUCAGCUGAUAUUUUAAA | 6195 | UUUAAAAUAUCAGCUGAGA |
| siRNA 706 | 706 | CUCAGCUGAUAUUUUAAAA | 6196 | UUUUAAAAUAUCAGCUGAG |
| siRNA 707 | 707 | UCAGCUGAUAUUUUAAAAU | 6197 | AUUUUAAAAUAUCAGCUGA |
| siRNA 708 | 708 | CAGCUGAUAUUUUAAAAUU | 6198 | AAUUUUAAAAUAUCAGCUG |
| siRNA 709 | 709 | AGCUGAUAUUUUAAAAUUG | 6199 | CAAUUUUAAAAUAUCAGCU |
| siRNA 710 | 710 | GCUGAUAUUUUAAAAUUGG | 6200 | CCAAUUUUAAAAUAUCAGC |
| siRNA 711 | 711 | CUGAUAUUUUAAAAUUGGC | 6201 | GCCAAUUUUAAAAUAUCAG |
| siRNA 712 | 712 | UGAUAUUUUAAAAUUGGCU | 6202 | AGCCAAUUUUAAAAUAUCA |
| siRNA 713 | 713 | GAUAUUUUAAAAUUGGCUG | 6203 | CAGCCAAUUUUAAAAUAUC |
| siRNA 714 | 714 | AUAUUUUAAAAUUGGCUGG | 6204 | CCAGCCAAUUUUAAAAUAU |
| siRNA 715 | 715 | UAUUUUAAAAUUGGCUGGU | 6205 | ACCAGCCAAUUUUAAAAUA |
| siRNA 716 | 716 | AUUUUAAAAUUGGCUGGUA | 6206 | UACCAGCCAAUUUUAAAAU |
| siRNA 717 | 717 | UUUUAAAAUUGGCUGGUAA | 6207 | UUACCAGCCAAUUUUAAAA |
| siRNA 718 | 718 | UUUAAAAUUGGCUGGUAAU | 6208 | AUUACCAGCCAAUUUUAAA |
| siRNA 719 | 719 | UUAAAAUUGGCUGGUAAUU | 6209 | AAUUACCAGCCAAUUUUAA |
| siRNA 720 | 720 | UAAAAUUGGCUGGUAAUUA | 6210 | UAAUUACCAGCCAAUUUUA |
| siRNA 721 | 721 | AAAAUUGGCUGGUAAUUAU | 6211 | AUAAUUACCAGCCAAUUUU |
| siRNA 722 | 722 | AAAUUGGCUGGUAAUUAUG | 6212 | CAUAAUUACCAGCCAAUUU |
| siRNA 723 | 723 | AAUUGGCUGGUAAUUAUGU | 6213 | ACAUAAUUACCAGCCAAUU |
| siRNA 724 | 724 | AUUGGCUGGUAAUUAUGUU | 6214 | AACAUAAUUACCAGCCAAU |
| siRNA 725 | 725 | UUGGCUGGUAAUUAUGUUU | 6215 | AAACAUAAUUACCAGCCAA |
| siRNA 726 | 726 | UGGCUGGUAAUUAUGUUUU | 6216 | AAAACAUAAUUACCAGCCA |
| siRNA 727 | 727 | GGCUGGUAAUUAUGUUUUU | 6217 | AAAAACAUAAUUACCAGCC |
| siRNA 728 | 728 | GCUGGUAAUUAUGUUUUUA | 6218 | UAAAAACAUAAUUACCAGC |
| siRNA 729 | 729 | CUGGUAAUUAUGUUUUUAA | 6219 | UUAAAAACAUAAUUACCAG |
| siRNA 730 | 730 | UGGUAAUUAUGUUUUUAAU | 6220 | AUUAAAAACAUAAUUACCA |
| siRNA 731 | 731 | GGUAAUUAUGUUUUUAAUA | 6221 | UAUUAAAAACAUAAUUACC |
| siRNA 732 | 732 | GUAAUUAUGUUUUUAAUAU | 6222 | AUAUUAAAAACAUAAUUAC |
| siRNA 733 | 733 | UAAUUAUGUUUUUAAUAUC | 6223 | GAUAUUAAAAACAUAAUUA |
| siRNA 734 | 734 | AAUUAUGUUUUUAAUAUCC | 6224 | GGAUAUUAAAAACAUAAUU |
| siRNA 735 | 735 | AUUAUGUUUUUAAUAUCCG | 6225 | CGGAUAUUAAAAACAUAAU |
| siRNA 736 | 736 | UUAUGUUUUUAAUAUCCGG | 6226 | CCGGAUAUUAAAAACAUAA |
| siRNA 737 | 737 | UAUGUUUUUAAUAUCCGGC | 6227 | GCCGGAUAUUAAAAACAUA |
| siRNA 738 | 738 | AUGUUUUUAAUAUCCGGCA | 6228 | UGCCGGAUAUUAAAAACAU |
| siRNA 739 | 739 | UGUUUUUAAUAUCCGGCAU | 6229 | AUGCCGGAUAUUAAAAACA |
| siRNA 740 | 740 | GUUUUUAAUAUCCGGCAUU | 6230 | AAUGCCGGAUAUUAAAAAC |
| siRNA 741 | 741 | UUUUUAAUAUCCGGCAUUA | 6231 | UAAUGCCGGAUAUUAAAAA |
| siRNA 742 | 742 | UUUUAAUAUCCGGCAUUAU | 6232 | AUAAUGCCGGAUAUUAAAA |
| siRNA 743 | 743 | UUUAAUAUCCGGCAUUAUG | 6233 | CAUAAUGCCGGAUAUUAAA |
| siRNA 744 | 744 | UUAAUAUCCGGCAUUAUGA | 6234 | UCAUAAUGCCGGAUAUUAA |
| siRNA 745 | 745 | UAAUAUCCGGCAUUAUGAA | 6235 | UUCAUAAUGCCGGAUAUUA |
| siRNA 746 | 746 | AAUAUCCGGCAUUAUGAAA | 6236 | UUUCAUAAUGCCGGAUAUU |
| siRNA 747 | 747 | AUAUCCGGCAUUAUGAAAU | 6237 | AUUUCAUAAUGCCGGAUAU |
| siRNA 748 | 748 | UAUCCGGCAUUAUGAAAUA | 6238 | UAUUUCAUAAUGCCGGAUA |
| siRNA 749 | 749 | AUCCGGCAUUAUGAAAUAU | 6239 | AUAUUUCAUAAUGCCGGAU |
| siRNA 750 | 750 | UCCGGCAUUAUGAAAUAUC | 6240 | GAUAUUUCAUAAUGCCGGA |
| siRNA 751 | 751 | CCGGCAUUAUGAAAUAUCU | 6241 | AGAUAUUUCAUAAUGCCGG |
| siRNA 752 | 752 | CGGCAUUAUGAAAUAUCUC | 6242 | GAGAUAUUUCAUAAUGCCG |
| siRNA 753 | 753 | GGCAUUAUGAAAUAUCUCA | 6243 | UGAGAUAUUUCAUAAUGCC |
| siRNA 754 | 754 | GCAUUAUGAAAUAUCUCAG | 6244 | CUGAGAUAUUUCAUAAUGC |
| siRNA 755 | 755 | CAUUAUGAAAUAUCUCAGC | 6245 | GCUGAGAUAUUUCAUAAUG |
| siRNA 756 | 756 | AUUAUGAAAUAUCUCAGCA | 6246 | UGCUGAGAUAUUUCAUAAU |
| siRNA 757 | 757 | UUAUGAAAUAUCUCAGCAG | 6247 | CUGCUGAGAUAUUUCAUAA |
| siRNA 758 | 758 | UAUGAAAUAUCUCAGCAGG | 6248 | CCUGCUGAGAUAUUUCAUA |
| siRNA 759 | 759 | AUGAAAUAUCUCAGCAGGA | 6249 | UCCUGCUGAGAUAUUUCAU |
| siRNA 760 | 760 | UGAAAUAUCUCAGCAGGAC | 6250 | GUCCUGCUGAGAUAUUUCA |
| siRNA 761 | 761 | GAAAUAUCUCAGCAGGACA | 6251 | UGUCCUGCUGAGAUAUUUC |
| siRNA 762 | 762 | AAAUAUCUCAGCAGGACAU | 6252 | AUGUCCUGCUGAGAUAUUU |
| siRNA 763 | 763 | AAUAUCUCAGCAGGACAUU | 6253 | AAUGUCCUGCUGAGAUAUU |
| siRNA 764 | 764 | AUAUCUCAGCAGGACAUUA | 6254 | UAAUGUCCUGCUGAGAUAU |
| siRNA 765 | 765 | UAUCUCAGCAGGACAUUAA | 6255 | UUAAUGUCCUGCUGAGAUA |
| siRNA 766 | 766 | AUCUCAGCAGGACAUUAAA | 6256 | UUUAAUGUCCUGCUGAGAU |
| siRNA 767 | 767 | UCUCAGCAGGACAUUAAAG | 6257 | CUUUAAUGUCCUGCUGAGA |
| siRNA 768 | 768 | CUCAGCAGGACAUUAAAGU | 6258 | ACUUUAAUGUCCUGCUGAG |
| siRNA 769 | 769 | UCAGCAGGACAUUAAAGUG | 6259 | CACUUUAAUGUCCUGCUGA |
| siRNA 770 | 770 | CAGCAGGACAUUAAAGUGU | 6260 | ACACUUUAAUGUCCUGCUG |
| siRNA 771 | 771 | AGCAGGACAUUAAAGUGUC | 6261 | GACACUUUAAUGUCCUGCU |
| siRNA 772 | 772 | GCAGGACAUUAAAGUGUCA | 6262 | UGACACUUUAAUGUCCUGC |
| siRNA 773 | 773 | CAGGACAUUAAAGUGUCAA | 6263 | UUGACACUUUAAUGUCCUG |
| siRNA 774 | 774 | AGGACAUUAAAGUGUCAAU | 6264 | AUUGACACUUUAAUGUCCU |
| siRNA 775 | 775 | GGACAUUAAAGUGUCAAUG | 6265 | CAUUGACACUUUAAUGUCC |
| siRNA 776 | 776 | GACAUUAAAGUGUCAAUGU | 6266 | ACAUUGACACUUUAAUGUC |
| siRNA 777 | 777 | ACAUUAAAGUGUCAAUGUG | 6267 | CACAUUGACACUUUAAUGU |
| siRNA 778 | 778 | CAUUAAAGUGUCAAUGUGU | 6268 | ACACAUUGACACUUUAAUG |
| siRNA 779 | 779 | AUUAAAGUGUCAAUGUGUG | 6269 | CACACAUUGACACUUUAAU |
| siRNA 780 | 780 | UUAAAGUGUCAAUGUGUGC | 6270 | GCACACAUUGACACUUUAA |
| siRNA 781 | 781 | UAAAGUGUCAAUGUGUGCG | 6271 | CGCACACAUUGACACUUUA |
| siRNA 782 | 782 | AAAGUGUCAAUGUGUGCGG | 6272 | CCGCACACAUUGACACUUU |
| siRNA 783 | 783 | AAGUGUCAAUGUGUGCGGA | 6273 | UCCGCACACAUUGACACUU |
| siRNA 784 | 784 | AGUGUCAAUGUGUGCGGAU | 6274 | AUCCGCACACAUUGACACU |
| siRNA 785 | 785 | GUGUCAAUGUGUGCGGAUA | 6275 | UAUCCGCACACAUUGACAC |
| siRNA 786 | 786 | UGUCAAUGUGUGCGGAUAA | 6276 | UUAUCCGCACACAUUGACA |
| siRNA 787 | 787 | GUCAAUGUGUGCGGAUAAG | 6277 | CUUAUCCGCACACAUUGAC |
| siRNA 788 | 788 | UCAAUGUGUGCGGAUAAGG | 6278 | CCUUAUCCGCACACAUUGA |
| siRNA 789 | 789 | CAAUGUGUGCGGAUAAGGU | 6279 | ACCUUAUCCGCACACAUUG |
| siRNA 790 | 790 | AAUGUGUGCGGAUAAGGUU | 6280 | AACCUUAUCCGCACACAUU |
| siRNA 791 | 791 | AUGUGUGCGGAUAAGGUUU | 6281 | AAACCUUAUCCGCACACAU |
| siRNA 792 | 792 | UGUGUGCGGAUAAGGUUUU | 6282 | AAAACCUUAUCCGCACACA |
| siRNA 793 | 793 | GUGUGCGGAUAAGGUUUUG | 6283 | CAAAACCUUAUCCGCACAC |
| siRNA 794 | 794 | UGUGCGGAUAAGGUUUUGA | 6284 | UCAAAACCUUAUCCGCACA |
| siRNA 795 | 795 | GUGCGGAUAAGGUUUUGAU | 6285 | AUCAAAACCUUAUCCGCAC |
| siRNA 796 | 796 | UGCGGAUAAGGUUUUGAUG | 6286 | CAUCAAAACCUUAUCCGCA |
| siRNA 797 | 797 | GCGGAUAAGGUUUUGAUGG | 6287 | CCAUCAAAACCUUAUCCGC |
| siRNA 798 | 798 | CGGAUAAGGUUUUGAUGGA | 6288 | UCCAUCAAAACCUUAUCCG |
| siRNA 799 | 799 | GGAUAAGGUUUUGAUGGAC | 6289 | GUCCAUCAAAACCUUAUCC |
| siRNA 800 | 800 | GAUAAGGUUUUGAUGGACA | 6290 | UGUCCAUCAAAACCUUAUC |
| siRNA 801 | 801 | AUAAGGUUUUGAUGGACAU | 6291 | AUGUCCAUCAAAACCUUAU |
| siRNA 802 | 802 | UAAGGUUUUGAUGGACAUG | 6292 | CAUGUCCAUCAAAACCUUA |
| siRNA 803 | 803 | AAGGUUUUGAUGGACAUGU | 6293 | ACAUGUCCAUCAAAACCUU |
| siRNA 804 | 804 | AGGUUUUGAUGGACAUGUU | 6294 | AACAUGUCCAUCAAAACCU |
| siRNA 805 | 805 | GGUUUUGAUGGACAUGUUU | 6295 | AAACAUGUCCAUCAAAACC |
| siRNA 806 | 806 | GUUUUGAUGGACAUGUUUG | 6296 | CAAACAUGUCCAUCAAAAC |
| siRNA 807 | 807 | UUUUGAUGGACAUGUUUGA | 6297 | UCAAACAUGUCCAUCAAAA |
| siRNA 808 | 808 | UUUGAUGGACAUGUUUGAU | 6298 | AUCAAACAUGUCCAUCAAA |
| siRNA 809 | 809 | UUGAUGGACAUGUUUGAUC | 6299 | GAUCAAACAUGUCCAUCAA |
| siRNA 810 | 810 | UGAUGGACAUGUUUGAUCA | 6300 | UGAUCAAACAUGUCCAUCA |
| siRNA 811 | 811 | GAUGGACAUGUUUGAUCAG | 6301 | CUGAUCAAACAUGUCCAUC |
| siRNA 812 | 812 | AUGGACAUGUUUGAUCAGG | 6302 | CCUGAUCAAACAUGUCCAU |
| siRNA 813 | 813 | UGGACAUGUUUGAUCAGGA | 6303 | UCCUGAUCAAACAUGUCCA |
| siRNA 814 | 814 | GGACAUGUUUGAUCAGGAU | 6304 | AUCCUGAUCAAACAUGUCC |
| siRNA 815 | 815 | GACAUGUUUGAUCAGGAUG | 6305 | CAUCCUGAUCAAACAUGUC |
| siRNA 816 | 816 | ACAUGUUUGAUCAGGAUGA | 6306 | UCAUCCUGAUCAAACAUGU |
| siRNA 817 | 817 | CAUGUUUGAUCAGGAUGAC | 6307 | GUCAUCCUGAUCAAACAUG |
| siRNA 818 | 818 | AUGUUUGAUCAGGAUGACA | 6308 | UGUCAUCCUGAUCAAACAU |
| siRNA 819 | 819 | UGUUUGAUCAGGAUGACAU | 6309 | AUGUCAUCCUGAUCAAACA |
| siRNA 820 | 820 | GUUUGAUCAGGAUGACAUA | 6310 | UAUGUCAUCCUGAUCAAAC |
| siRNA 821 | 821 | UUUGAUCAGGAUGACAUAG | 6311 | CUAUGUCAUCCUGAUCAAA |
| siRNA 822 | 822 | UUGAUCAGGAUGACAUAGG | 6312 | CCUAUGUCAUCCUGAUCAA |
| siRNA 823 | 823 | UGAUCAGGAUGACAUAGGU | 6313 | ACCUAUGUCAUCCUGAUCA |
| siRNA 824 | 824 | GAUCAGGAUGACAUAGGUU | 6314 | AACCUAUGUCAUCCUGAUC |
| siRNA 825 | 825 | AUCAGGAUGACAUAGGUUU | 6315 | AAACCUAUGUCAUCCUGAU |
| siRNA 826 | 826 | UCAGGAUGACAUAGGUUUG | 6316 | CAAACCUAUGUCAUCCUGA |
| siRNA 827 | 827 | CAGGAUGACAUAGGUUUGG | 6317 | CCAAACCUAUGUCAUCCUG |
| siRNA 828 | 828 | AGGAUGACAUAGGUUUGGU | 6318 | ACCAAACCUAUGUCAUCCU |
| siRNA 829 | 829 | GGAUGACAUAGGUUUGGUU | 6319 | AACCAAACCUAUGUCAUCC |
| siRNA 830 | 830 | GAUGACAUAGGUUUGGUUU | 6320 | AAACCAAACCUAUGUCAUC |
| siRNA 831 | 831 | AUGACAUAGGUUUGGUUUC | 6321 | GAAACCAAACCUAUGUCAU |
| siRNA 832 | 832 | UGACAUAGGUUUGGUUUCU | 6322 | AGAAACCAAACCUAUGUCA |
| siRNA 833 | 833 | GACAUAGGUUUGGUUUCUC | 6323 | GAGAAACCAAACCUAUGUC |
| siRNA 834 | 834 | ACAUAGGUUUGGUUUCUCU | 6324 | AGAGAAACCAAACCUAUGU |
| siRNA 835 | 835 | CAUAGGUUUGGUUUCUCUC | 6325 | GAGAGAAACCAAACCUAUG |
| siRNA 836 | 836 | AUAGGUUUGGUUUCUCUCU | 6326 | AGAGAGAAACCAAACCUAU |
| siRNA 837 | 837 | UAGGUUUGGUUUCUCUCUG | 6327 | CAGAGAGAAACCAAACCUA |
| siRNA 838 | 838 | AGGUUUGGUUUCUCUCUGU | 6328 | ACAGAGAGAAACCAAACCU |
| siRNA 839 | 839 | GGUUUGGUUUCUCUCUGUG | 6329 | CACAGAGAGAAACCAAACC |
| siRNA 840 | 840 | GUUUGGUUUCUCUCUGUGA | 6330 | UCACAGAGAGAAACCAAAC |
| siRNA 841 | 841 | UUUGGUUUCUCUCUGUGAA | 6331 | UUCACAGAGAGAAACCAAA |
| siRNA 842 | 842 | UUGGUUUCUCUCUGUGAAG | 6332 | CUUCACAGAGAGAAACCAA |
| siRNA 843 | 843 | UGGUUUCUCUCUGUGAAGA | 6333 | UCUUCACAGAGAGAAACCA |
| siRNA 844 | 844 | GGUUUCUCUCUGUGAAGAU | 6334 | AUCUUCACAGAGAGAAACC |
| siRNA 845 | 845 | GUUUCUCUCUGUGAAGAUG | 6335 | CAUCUUCACAGAGAGAAAC |
| siRNA 846 | 846 | UUUCUCUCUGUGAAGAUGA | 6336 | UCAUCUUCACAGAGAGAAA |
| siRNA 847 | 847 | UUCUCUCUGUGAAGAUGAA | 6337 | UUCAUCUUCACAGAGAGAA |
| siRNA 848 | 848 | UCUCUCUGUGAAGAUGAAC | 6338 | GUUCAUCUUCACAGAGAGA |
| siRNA 849 | 849 | CUCUCUGUGAAGAUGAACC | 6339 | GGUUCAUCUUCACAGAGAG |
| siRNA 850 | 850 | UCUCUGUGAAGAUGAACCU | 6340 | AGGUUCAUCUUCACAGAGA |
| siRNA 851 | 851 | CUCUGUGAAGAUGAACCUA | 6341 | UAGGUUCAUCUUCACAGAG |
| siRNA 852 | 852 | UCUGUGAAGAUGAACCUAG | 6342 | CUAGGUUCAUCUUCACAGA |
| siRNA 853 | 853 | CUGUGAAGAUGAACCUAGU | 6343 | ACUAGGUUCAUCUUCACAG |
| siRNA 854 | 854 | UGUGAAGAUGAACCUAGUU | 6344 | AACUAGGUUCAUCUUCACA |
| siRNA 855 | 855 | GUGAAGAUGAACCUAGUUC | 6345 | GAACUAGGUUCAUCUUCAC |
| siRNA 856 | 856 | UGAAGAUGAACCUAGUUCU | 6346 | AGAACUAGGUUCAUCUUCA |
| siRNA 857 | 857 | GAAGAUGAACCUAGUUCUU | 6347 | AAGAACUAGGUUCAUCUUC |
| siRNA 858 | 858 | AAGAUGAACCUAGUUCUUC | 6348 | GAAGAACUAGGUUCAUCUU |
| siRNA 859 | 859 | AGAUGAACCUAGUUCUUCU | 6349 | AGAAGAACUAGGUUCAUCU |
| siRNA 860 | 860 | GAUGAACCUAGUUCUUCUG | 6350 | CAGAAGAACUAGGUUCAUC |
| siRNA 861 | 861 | AUGAACCUAGUUCUUCUGG | 6351 | CCAGAAGAACUAGGUUCAU |
| siRNA 862 | 862 | UGAACCUAGUUCUUCUGGU | 6352 | ACCAGAAGAACUAGGUUCA |
| siRNA 863 | 863 | GAACCUAGUUCUUCUGGUG | 6353 | CACCAGAAGAACUAGGUUC |
| siRNA 864 | 864 | AACCUAGUUCUUCUGGUGA | 6354 | UCACCAGAAGAACUAGGUU |
| siRNA 865 | 865 | ACCUAGUUCUUCUGGUGAA | 6355 | UUCACCAGAAGAACUAGGU |
| siRNA 866 | 866 | CCUAGUUCUUCUGGUGAAU | 6356 | AUUCACCAGAAGAACUAGG |
| siRNA 867 | 867 | CUAGUUCUUCUGGUGAAUU | 6357 | AAUUCACCAGAAGAACUAG |
| siRNA 868 | 868 | UAGUUCUUCUGGUGAAUUA | 6358 | UAAUUCACCAGAAGAACUA |
| siRNA 869 | 869 | AGUUCUUCUGGUGAAUUAA | 6359 | UUAAUUCACCAGAAGAACU |
| siRNA 870 | 870 | GUUCUUCUGGUGAAUUAAA | 6360 | UUUAAUUCACCAGAAGAAC |
| siRNA 871 | 871 | UUCUUCUGGUGAAUUAAAC | 6361 | GUUUAAUUCACCAGAAGAA |
| siRNA 872 | 872 | UCUUCUGGUGAAUUAAACU | 6362 | AGUUUAAUUCACCAGAAGA |
| siRNA 873 | 873 | CUUCUGGUGAAUUAAACUA | 6363 | UAGUUUAAUUCACCAGAAG |
| siRNA 874 | 874 | UUCUGGUGAAUUAAACUAC | 6364 | GUAGUUUAAUUCACCAGAA |
| siRNA 875 | 875 | UCUGGUGAAUUAAACUACU | 6365 | AGUAGUUUAAUUCACCAGA |
| siRNA 876 | 876 | CUGGUGAAUUAAACUACUA | 6366 | UAGUAGUUUAAUUCACCAG |
| siRNA 877 | 877 | UGGUGAAUUAAACUACUAU | 6367 | AUAGUAGUUUAAUUCACCA |
| siRNA 878 | 878 | GGUGAAUUAAACUACUAUG | 6368 | CAUAGUAGUUUAAUUCACC |
| siRNA 879 | 879 | GUGAAUUAAACUACUAUGA | 6369 | UCAUAGUAGUUUAAUUCAC |
| siRNA 880 | 880 | UGAAUUAAACUACUAUGAU | 6370 | AUCAUAGUAGUUUAAUUCA |
| siRNA 881 | 881 | GAAUUAAACUACUAUGAUC | 6371 | GAUCAUAGUAGUUUAAUUC |
| siRNA 882 | 882 | AAUUAAACUACUAUGAUCU | 6372 | AGAUCAUAGUAGUUUAAUU |
| siRNA 883 | 883 | AUUAAACUACUAUGAUCUU | 6373 | AAGAUCAUAGUAGUUUAAU |
| siRNA 884 | 884 | UUAAACUACUAUGAUCUUG | 6374 | CAAGAUCAUAGUAGUUUAA |
| siRNA 885 | 885 | UAAACUACUAUGAUCUUGU | 6375 | ACAAGAUCAUAGUAGUUUA |
| siRNA 886 | 886 | AAACUACUAUGAUCUUGUC | 6376 | GACAAGAUCAUAGUAGUUU |
| siRNA 887 | 887 | AACUACUAUGAUCUUGUCA | 6377 | UGACAAGAUCAUAGUAGUU |
| siRNA 888 | 888 | ACUACUAUGAUCUUGUCAG | 6378 | CUGACAAGAUCAUAGUAGU |
| siRNA 889 | 889 | CUACUAUGAUCUUGUCAGA | 6379 | UCUGACAAGAUCAUAGUAG |
| siRNA 890 | 890 | UACUAUGAUCUUGUCAGAA | 6380 | UUCUGACAAGAUCAUAGUA |
| siRNA 891 | 891 | ACUAUGAUCUUGUCAGAAC | 6381 | GUUCUGACAAGAUCAUAGU |
| siRNA 892 | 892 | CUAUGAUCUUGUCAGAACU | 6382 | AGUUCUGACAAGAUCAUAG |
| siRNA 893 | 893 | UAUGAUCUUGUCAGAACUG | 6383 | CAGUUCUGACAAGAUCAUA |
| siRNA 894 | 894 | AUGAUCUUGUCAGAACUGA | 6384 | UCAGUUCUGACAAGAUCAU |
| siRNA 895 | 895 | UGAUCUUGUCAGAACUGAA | 6385 | UUCAGUUCUGACAAGAUCA |
| siRNA 896 | 896 | GAUCUUGUCAGAACUGAAA | 6386 | UUUCAGUUCUGACAAGAUC |
| siRNA 897 | 897 | AUCUUGUCAGAACUGAAAU | 6387 | AUUUCAGUUCUGACAAGAU |
| siRNA 898 | 898 | UCUUGUCAGAACUGAAAUC | 6388 | GAUUUCAGUUCUGACAAGA |
| siRNA 899 | 899 | CUUGUCAGAACUGAAAUCG | 6389 | CGAUUUCAGUUCUGACAAG |
| siRNA 900 | 900 | UUGUCAGAACUGAAAUCGC | 6390 | GCGAUUUCAGUUCUGACAA |
| siRNA 901 | 901 | UGUCAGAACUGAAAUCGCA | 6391 | UGCGAUUUCAGUUCUGACA |
| siRNA 902 | 902 | GUCAGAACUGAAAUCGCAG | 6392 | CUGCGAUUUCAGUUCUGAC |
| siRNA 903 | 903 | UCAGAACUGAAAUCGCAGA | 6393 | UCUGCGAUUUCAGUUCUGA |
| siRNA 904 | 904 | CAGAACUGAAAUCGCAGAA | 6394 | UUCUGCGAUUUCAGUUCUG |
| siRNA 905 | 905 | AGAACUGAAAUCGCAGAAG | 6395 | CUUCUGCGAUUUCAGUUCU |
| siRNA 906 | 906 | GAACUGAAAUCGCAGAAGA | 6396 | UCUUCUGCGAUUUCAGUUC |
| siRNA 907 | 907 | AACUGAAAUCGCAGAAGAA | 6397 | UUCUUCUGCGAUUUCAGUU |
| siRNA 908 | 908 | ACUGAAAUCGCAGAAGAAA | 6398 | UUUCUUCUGCGAUUUCAGU |
| siRNA 909 | 909 | CUGAAAUCGCAGAAGAAAG | 6399 | CUUUCUUCUGCGAUUUCAG |
| siRNA 910 | 910 | UGAAAUCGCAGAAGAAAGA | 6400 | UCUUUCUUCUGCGAUUUCA |
| siRNA 911 | 911 | GAAAUCGCAGAAGAAAGAC | 6401 | GUCUUUCUUCUGCGAUUUC |
| siRNA 912 | 912 | AAAUCGCAGAAGAAAGACA | 6402 | UGUCUUUCUUCUGCGAUUU |
| siRNA 913 | 913 | AAUCGCAGAAGAAAGACAG | 6403 | CUGUCUUUCUUCUGCGAUU |
| siRNA 914 | 914 | AUCGCAGAAGAAAGACAGU | 6404 | ACUGUCUUUCUUCUGCGAU |
| siRNA 915 | 915 | UCGCAGAAGAAAGACAGUA | 6405 | UACUGUCUUUCUUCUGCGA |
| siRNA 916 | 916 | CGCAGAAGAAAGACAGUAU | 6406 | AUACUGUCUUUCUUCUGCG |
| siRNA 917 | 917 | GCAGAAGAAAGACAGUAUC | 6407 | GAUACUGUCUUUCUUCUGC |
| siRNA 918 | 918 | CAGAAGAAAGACAGUAUCU | 6408 | AGAUACUGUCUUUCUUCUG |
| siRNA 919 | 919 | AGAAGAAAGACAGUAUCUA | 6409 | UAGAUACUGUCUUUCUUCU |
| siRNA 920 | 920 | GAAGAAAGACAGUAUCUAC | 6410 | GUAGAUACUGUCUUUCUUC |
| siRNA 921 | 921 | AAGAAAGACAGUAUCUACG | 6411 | CGUAGAUACUGUCUUUCUU |
| siRNA 922 | 922 | AGAAAGACAGUAUCUACGG | 6412 | CCGUAGAUACUGUCUUUCU |
| siRNA 923 | 923 | GAAAGACAGUAUCUACGGG | 6413 | CCCGUAGAUACUGUCUUUC |
| siRNA 924 | 924 | AAAGACAGUAUCUACGGGA | 6414 | UCCCGUAGAUACUGUCUUU |
| siRNA 925 | 925 | AAGACAGUAUCUACGGGAA | 6415 | UUCCCGUAGAUACUGUCUU |
| siRNA 926 | 926 | AGACAGUAUCUACGGGAAU | 6416 | AUUCCCGUAGAUACUGUCU |
| siRNA 927 | 927 | GACAGUAUCUACGGGAAUU | 6417 | AAUUCCCGUAGAUACUGUC |
| siRNA 928 | 928 | ACAGUAUCUACGGGAAUUA | 6418 | UAAUUCCCGUAGAUACUGU |
| siRNA 929 | 929 | CAGUAUCUACGGGAAUUAA | 6419 | UUAAUUCCCGUAGAUACUG |
| siRNA 930 | 930 | AGUAUCUACGGGAAUUAAA | 6420 | UUUAAUUCCCGUAGAUACU |
| siRNA 931 | 931 | GUAUCUACGGGAAUUAAAU | 6421 | AUUUAAUUCCCGUAGAUAC |
| siRNA 932 | 932 | UAUCUACGGGAAUUAAAUA | 6422 | UAUUUAAUUCCCGUAGAUA |
| siRNA 933 | 933 | AUCUACGGGAAUUAAAUAU | 6423 | AUAUUUAAUUCCCGUAGAU |
| siRNA 934 | 934 | UCUACGGGAAUUAAAUAUG | 6424 | CAUAUUUAAUUCCCGUAGA |
| siRNA 935 | 935 | CUACGGGAAUUAAAUAUGA | 6425 | UCAUAUUUAAUUCCCGUAG |
| siRNA 936 | 936 | UACGGGAAUUAAAUAUGAU | 6426 | AUCAUAUUUAAUUCCCGUA |
| siRNA 937 | 937 | ACGGGAAUUAAAUAUGAUC | 6427 | GAUCAUAUUUAAUUCCCGU |
| siRNA 938 | 938 | CGGGAAUUAAAUAUGAUCA | 6428 | UGAUCAUAUUUAAUUCCCG |
| siRNA 939 | 939 | GGGAAUUAAAUAUGAUCAU | 6429 | AUGAUCAUAUUUAAUUCCC |
| siRNA 940 | 940 | GGAAUUAAAUAUGAUCAUA | 6430 | UAUGAUCAUAUUUAAUUCC |
| siRNA 941 | 941 | GAAUUAAAUAUGAUCAUAA | 6431 | UUAUGAUCAUAUUUAAUUC |
| siRNA 942 | 942 | AAUUAAAUAUGAUCAUAAA | 6432 | UUUAUGAUCAUAUUUAAUU |
| siRNA 943 | 943 | AUUAAAUAUGAUCAUAAAA | 6433 | UUUUAUGAUCAUAUUUAAU |
| siRNA 944 | 944 | UUAAAUAUGAUCAUAAAAG | 6434 | CUUUUAUGAUCAUAUUUAA |
| siRNA 945 | 945 | UAAAUAUGAUCAUAAAAGU | 6435 | ACUUUUAUGAUCAUAUUUA |
| siRNA 946 | 946 | AAAUAUGAUCAUAAAAGUG | 6436 | CACUUUUAUGAUCAUAUUU |
| siRNA 947 | 947 | AAUAUGAUCAUAAAAGUGU | 6437 | ACACUUUUAUGAUCAUAUU |
| siRNA 948 | 948 | AUAUGAUCAUAAAAGUGUU | 6438 | AACACUUUUAUGAUCAUAU |
| siRNA 949 | 949 | UAUGAUCAUAAAAGUGUUU | 6439 | AAACACUUUUAUGAUCAUA |
| siRNA 950 | 950 | AUGAUCAUAAAAGUGUUUC | 6440 | GAAACACUUUUAUGAUCAU |
| siRNA 951 | 951 | UGAUCAUAAAAGUGUUUCG | 6441 | CGAAACACUUUUAUGAUCA |
| siRNA 952 | 952 | GAUCAUAAAAGUGUUUCGA | 6442 | UCGAAACACUUUUAUGAUC |
| siRNA 953 | 953 | AUCAUAAAAGUGUUUCGAG | 6443 | CUCGAAACACUUUUAUGAU |
| siRNA 954 | 954 | UCAUAAAAGUGUUUCGAGA | 6444 | UCUCGAAACACUUUUAUGA |
| siRNA 955 | 955 | CAUAAAAGUGUUUCGAGAA | 6445 | UUCUCGAAACACUUUUAUG |
| siRNA 956 | 956 | AUAAAAGUGUUUCGAGAAG | 6446 | CUUCUCGAAACACUUUUAU |
| siRNA 957 | 957 | UAAAAGUGUUUCGAGAAGC | 6447 | GCUUCUCGAAACACUUUUA |
| siRNA 958 | 958 | AAAAGUGUUUCGAGAAGCC | 6448 | GGCUUCUCGAAACACUUUU |
| siRNA 959 | 959 | AAAGUGUUUCGAGAAGCCU | 6449 | AGGCUUCUCGAAACACUUU |
| siRNA 960 | 960 | AAGUGUUUCGAGAAGCCUU | 6450 | AAGGCUUCUCGAAACACUU |
| siRNA 961 | 961 | AGUGUUUCGAGAAGCCUUU | 6451 | AAAGGCUUCUCGAAACACU |
| siRNA 962 | 962 | GUGUUUCGAGAAGCCUUUC | 6452 | GAAAGGCUUCUCGAAACAC |
| siRNA 963 | 963 | UGUUUCGAGAAGCCUUUCU | 6453 | AGAAAGGCUUCUCGAAACA |
| siRNA 964 | 964 | GUUUCGAGAAGCCUUUCUU | 6454 | AAGAAAGGCUUCUCGAAAC |
| siRNA 965 | 965 | UUUCGAGAAGCCUUUCUUU | 6455 | AAAGAAAGGCUUCUCGAAA |
| siRNA 966 | 966 | UUCGAGAAGCCUUUCUUUC | 6456 | GAAAGAAAGGCUUCUCGAA |
| siRNA 967 | 967 | UCGAGAAGCCUUUCUUUCU | 6457 | AGAAAGAAAGGCUUCUCGA |
| siRNA 968 | 968 | CGAGAAGCCUUUCUUUCUG | 6458 | CAGAAAGAAAGGCUUCUCG |
| siRNA 969 | 969 | GAGAAGCCUUUCUUUCUGA | 6459 | UCAGAAAGAAAGGCUUCUC |
| siRNA 970 | 970 | AGAAGCCUUUCUUUCUGAU | 6460 | AUCAGAAAGAAAGGCUUCU |
| siRNA 971 | 971 | GAAGCCUUUCUUUCUGAUA | 6461 | UAUCAGAAAGAAAGGCUUC |
| siRNA 972 | 972 | AAGCCUUUCUUUCUGAUAG | 6462 | CUAUCAGAAAGAAAGGCUU |
| siRNA 973 | 973 | AGCCUUUCUUUCUGAUAGA | 6463 | UCUAUCAGAAAGAAAGGCU |
| siRNA 974 | 974 | GCCUUUCUUUCUGAUAGAA | 6464 | UUCUAUCAGAAAGAAAGGC |
| siRNA 975 | 975 | CCUUUCUUUCUGAUAGAAA | 6465 | UUUCUAUCAGAAAGAAAGG |
| siRNA 976 | 976 | CUUUCUUUCUGAUAGAAAG | 6466 | CUUUCUAUCAGAAAGAAAG |
| siRNA 977 | 977 | UUUCUUUCUGAUAGAAAGC | 6467 | GCUUUCUAUCAGAAAGAAA |
| siRNA 978 | 978 | UUCUUUCUGAUAGAAAGCU | 6468 | AGCUUUCUAUCAGAAAGAA |
| siRNA 979 | 979 | UCUUUCUGAUAGAAAGCUG | 6469 | CAGCUUUCUAUCAGAAAGA |
| siRNA 980 | 980 | CUUUCUGAUAGAAAGCUGU | 6470 | ACAGCUUUCUAUCAGAAAG |
| siRNA 981 | 981 | UUUCUGAUAGAAAGCUGUU | 6471 | AACAGCUUUCUAUCAGAAA |
| siRNA 982 | 982 | UUCUGAUAGAAAGCUGUUU | 6472 | AAACAGCUUUCUAUCAGAA |
| siRNA 983 | 983 | UCUGAUAGAAAGCUGUUUA | 6473 | UAAACAGCUUUCUAUCAGA |
| siRNA 984 | 984 | CUGAUAGAAAGCUGUUUAA | 6474 | UUAAACAGCUUUCUAUCAG |
| siRNA 985 | 985 | UGAUAGAAAGCUGUUUAAA | 6475 | UUUAAACAGCUUUCUAUCA |
| siRNA 986 | 986 | GAUAGAAAGCUGUUUAAAC | 6476 | GUUUAAACAGCUUUCUAUC |
| siRNA 987 | 987 | AUAGAAAGCUGUUUAAACC | 6477 | GGUUUAAACAGCUUUCUAU |
| siRNA 988 | 988 | UAGAAAGCUGUUUAAACCU | 6478 | AGGUUUAAACAGCUUUCUA |
| siRNA 989 | 989 | AGAAAGCUGUUUAAACCUU | 6479 | AAGGUUUAAACAGCUUUCU |
| siRNA 990 | 990 | GAAAGCUGUUUAAACCUUC | 6480 | GAAGGUUUAAACAGCUUUC |
| siRNA 991 | 991 | AAAGCUGUUUAAACCUUCU | 6481 | AGAAGGUUUAAACAGCUUU |
| siRNA 992 | 992 | AAGCUGUUUAAACCUUCUG | 6482 | CAGAAGGUUUAAACAGCUU |
| siRNA 993 | 993 | AGCUGUUUAAACCUUCUGA | 6483 | UCAGAAGGUUUAAACAGCU |
| siRNA 994 | 994 | GCUGUUUAAACCUUCUGAU | 6484 | AUCAGAAGGUUUAAACAGC |
| siRNA 995 | 995 | CUGUUUAAACCUUCUGAUA | 6485 | UAUCAGAAGGUUUAAACAG |
| siRNA 996 | 996 | UGUUUAAACCUUCUGAUAU | 6486 | AUAUCAGAAGGUUUAAACA |
| siRNA 997 | 997 | GUUUAAACCUUCUGAUAUC | 6487 | GAUAUCAGAAGGUUUAAAC |
| siRNA 998 | 998 | UUUAAACCUUCUGAUAUCG | 6488 | CGAUAUCAGAAGGUUUAAA |
| siRNA 999 | 999 | UUAAACCUUCUGAUAUCGA | 6489 | UCGAUAUCAGAAGGUUUAA |
| siRNA 1000 | 1000 | UAAACCUUCUGAUAUCGAA | 6490 | UUCGAUAUCAGAAGGUUUA |
| siRNA 1001 | 1001 | AAACCUUCUGAUAUCGAAA | 6491 | UUUCGAUAUCAGAAGGUUU |
| siRNA 1002 | 1002 | AACCUUCUGAUAUCGAAAA | 6492 | UUUUCGAUAUCAGAAGGUU |
| siRNA 1003 | 1003 | ACCUUCUGAUAUCGAAAAG | 6493 | CUUUUCGAUAUCAGAAGGU |
| siRNA 1004 | 1004 | CCUUCUGAUAUCGAAAAGA | 6494 | UCUUUUCGAUAUCAGAAGG |
| siRNA 1005 | 1005 | CUUCUGAUAUCGAAAAGAU | 6495 | AUCUUUUCGAUAUCAGAAG |
| siRNA 1006 | 1006 | UUCUGAUAUCGAAAAGAUU | 6496 | AAUCUUUUCGAUAUCAGAA |
| siRNA 1007 | 1007 | UCUGAUAUCGAAAAGAUUU | 6497 | AAAUCUUUUCGAUAUCAGA |
| siRNA 1008 | 1008 | CUGAUAUCGAAAAGAUUUU | 6498 | AAAAUCUUUUCGAUAUCAG |
| siRNA 1009 | 1009 | UGAUAUCGAAAAGAUUUUU | 6499 | AAAAAUCUUUUCGAUAUCA |
| siRNA 1010 | 1010 | GAUAUCGAAAAGAUUUUUA | 6500 | UAAAAAUCUUUUCGAUAUC |
| siRNA 1011 | 1011 | AUAUCGAAAAGAUUUUUAG | 6501 | CUAAAAAUCUUUUCGAUAU |
| siRNA 1012 | 1012 | UAUCGAAAAGAUUUUUAGU | 6502 | ACUAAAAAUCUUUUCGAUA |
| siRNA 1013 | 1013 | AUCGAAAAGAUUUUUAGUA | 6503 | UACUAAAAAUCUUUUCGAU |
| siRNA 1014 | 1014 | UCGAAAAGAUUUUUAGUAA | 6504 | UUACUAAAAAUCUUUUCGA |
| siRNA 1015 | 1015 | CGAAAAGAUUUUUAGUAAC | 6505 | GUUACUAAAAAUCUUUUCG |
| siRNA 1016 | 1016 | GAAAAGAUUUUUAGUAACA | 6506 | UGUUACUAAAAAUCUUUUC |
| siRNA 1017 | 1017 | AAAAGAUUUUUAGUAACAU | 6507 | AUGUUACUAAAAAUCUUUU |
| siRNA 1018 | 1018 | AAAGAUUUUUAGUAACAUU | 6508 | AAUGUUACUAAAAAUCUUU |
| siRNA 1019 | 1019 | AAGAUUUUUAGUAACAUUU | 6509 | AAAUGUUACUAAAAAUCUU |
| siRNA 1020 | 1020 | AGAUUUUUAGUAACAUUUC | 6510 | GAAAUGUUACUAAAAAUCU |
| siRNA 1021 | 1021 | GAUUUUUAGUAACAUUUCA | 6511 | UGAAAUGUUACUAAAAAUC |
| siRNA 1022 | 1022 | AUUUUUAGUAACAUUUCAG | 6512 | CUGAAAUGUUACUAAAAAU |
| siRNA 1023 | 1023 | UUUUUAGUAACAUUUCAGA | 6513 | UCUGAAAUGUUACUAAAAA |
| siRNA 1024 | 1024 | UUUUAGUAACAUUUCAGAU | 6514 | AUCUGAAAUGUUACUAAAA |
| siRNA 1025 | 1025 | UUUAGUAACAUUUCAGAUA | 6515 | UAUCUGAAAUGUUACUAAA |
| siRNA 1026 | 1026 | UUAGUAACAUUUCAGAUAU | 6516 | AUAUCUGAAAUGUUACUAA |
| siRNA 1027 | 1027 | UAGUAACAUUUCAGAUAUA | 6517 | UAUAUCUGAAAUGUUACUA |
| siRNA 1028 | 1028 | AGUAACAUUUCAGAUAUAC | 6518 | GUAUAUCUGAAAUGUUACU |
| siRNA 1029 | 1029 | GUAACAUUUCAGAUAUACA | 6519 | UGUAUAUCUGAAAUGUUAC |
| siRNA 1030 | 1030 | UAACAUUUCAGAUAUACAU | 6520 | AUGUAUAUCUGAAAUGUUA |
| siRNA 1031 | 1031 | AACAUUUCAGAUAUACAUG | 6521 | CAUGUAUAUCUGAAAUGUU |
| siRNA 1032 | 1032 | ACAUUUCAGAUAUACAUGA | 6522 | UCAUGUAUAUCUGAAAUGU |
| siRNA 1033 | 1033 | CAUUUCAGAUAUACAUGAA | 6523 | UUCAUGUAUAUCUGAAAUG |
| siRNA 1034 | 1034 | AUUUCAGAUAUACAUGAAU | 6524 | AUUCAUGUAUAUCUGAAAU |
| siRNA 1035 | 1035 | UUUCAGAUAUACAUGAAUU | 6525 | AAUUCAUGUAUAUCUGAAA |
| siRNA 1036 | 1036 | UUCAGAUAUACAUGAAUUG | 6526 | CAAUUCAUGUAUAUCUGAA |
| siRNA 1037 | 1037 | UCAGAUAUACAUGAAUUGA | 6527 | UCAAUUCAUGUAUAUCUGA |
| siRNA 1038 | 1038 | CAGAUAUACAUGAAUUGAC | 6528 | GUCAAUUCAUGUAUAUCUG |
| siRNA 1039 | 1039 | AGAUAUACAUGAAUUGACU | 6529 | AGUCAAUUCAUGUAUAUCU |
| siRNA 1040 | 1040 | GAUAUACAUGAAUUGACUG | 6530 | CAGUCAAUUCAUGUAUAUC |
| siRNA 1041 | 1041 | AUAUACAUGAAUUGACUGU | 6531 | ACAGUCAAUUCAUGUAUAU |
| siRNA 1042 | 1042 | UAUACAUGAAUUGACUGUG | 6532 | CACAGUCAAUUCAUGUAUA |
| siRNA 1043 | 1043 | AUACAUGAAUUGACUGUGA | 6533 | UCACAGUCAAUUCAUGUAU |
| siRNA 1044 | 1044 | UACAUGAAUUGACUGUGAA | 6534 | UUCACAGUCAAUUCAUGUA |
| siRNA 1045 | 1045 | ACAUGAAUUGACUGUGAAA | 6535 | UUUCACAGUCAAUUCAUGU |
| siRNA 1046 | 1046 | CAUGAAUUGACUGUGAAAC | 6536 | GUUUCACAGUCAAUUCAUG |
| siRNA 1047 | 1047 | AUGAAUUGACUGUGAAACU | 6537 | AGUUUCACAGUCAAUUCAU |
| siRNA 1048 | 1048 | UGAAUUGACUGUGAAACUU | 6538 | AAGUUUCACAGUCAAUUCA |
| siRNA 1049 | 1049 | GAAUUGACUGUGAAACUUU | 6539 | AAAGUUUCACAGUCAAUUC |
| siRNA 1050 | 1050 | AAUUGACUGUGAAACUUUU | 6540 | AAAAGUUUCACAGUCAAUU |
| siRNA 1051 | 1051 | AUUGACUGUGAAACUUUUA | 6541 | UAAAAGUUUCACAGUCAAU |
| siRNA 1052 | 1052 | UUGACUGUGAAACUUUUAG | 6542 | CUAAAAGUUUCACAGUCAA |
| siRNA 1053 | 1053 | UGACUGUGAAACUUUUAGG | 6543 | CCUAAAAGUUUCACAGUCA |
| siRNA 1054 | 1054 | GACUGUGAAACUUUUAGGU | 6544 | ACCUAAAAGUUUCACAGUC |
| siRNA 1055 | 1055 | ACUGUGAAACUUUUAGGUU | 6545 | AACCUAAAAGUUUCACAGU |
| siRNA 1056 | 1056 | CUGUGAAACUUUUAGGUUU | 6546 | AAACCUAAAAGUUUCACAG |
| siRNA 1057 | 1057 | UGUGAAACUUUUAGGUUUG | 6547 | CAAACCUAAAAGUUUCACA |
| siRNA 1058 | 1058 | GUGAAACUUUUAGGUUUGA | 6548 | UCAAACCUAAAAGUUUCAC |
| siRNA 1059 | 1059 | UGAAACUUUUAGGUUUGAU | 6549 | AUCAAACCUAAAAGUUUCA |
| siRNA 1060 | 1060 | GAAACUUUUAGGUUUGAUU | 6550 | AAUCAAACCUAAAAGUUUC |
| siRNA 1061 | 1061 | AAACUUUUAGGUUUGAUUG | 6551 | CAAUCAAACCUAAAAGUUU |
| siRNA 1062 | 1062 | AACUUUUAGGUUUGAUUGA | 6552 | UCAAUCAAACCUAAAAGUU |
| siRNA 1063 | 1063 | ACUUUUAGGUUUGAUUGAA | 6553 | UUCAAUCAAACCUAAAAGU |
| siRNA 1064 | 1064 | CUUUUAGGUUUGAUUGAAG | 6554 | CUUCAAUCAAACCUAAAAG |
| siRNA 1065 | 1065 | UUUUAGGUUUGAUUGAAGA | 6555 | UCUUCAAUCAAACCUAAAA |
| siRNA 1066 | 1066 | UUUAGGUUUGAUUGAAGAC | 6556 | GUCUUCAAUCAAACCUAAA |
| siRNA 1067 | 1067 | UUAGGUUUGAUUGAAGACA | 6557 | UGUCUUCAAUCAAACCUAA |
| siRNA 1068 | 1068 | UAGGUUUGAUUGAAGACAC | 6558 | GUGUCUUCAAUCAAACCUA |
| siRNA 1069 | 1069 | AGGUUUGAUUGAAGACACA | 6559 | UGUGUCUUCAAUCAAACCU |
| siRNA 1070 | 1070 | GGUUUGAUUGAAGACACAG | 6560 | CUGUGUCUUCAAUCAAACC |
| siRNA 1071 | 1071 | GUUUGAUUGAAGACACAGU | 6561 | ACUGUGUCUUCAAUCAAAC |
| siRNA 1072 | 1072 | UUUGAUUGAAGACACAGUU | 6562 | AACUGUGUCUUCAAUCAAA |
| siRNA 1073 | 1073 | UUGAUUGAAGACACAGUUG | 6563 | CAACUGUGUCUUCAAUCAA |
| siRNA 1074 | 1074 | UGAUUGAAGACACAGUUGA | 6564 | UCAACUGUGUCUUCAAUCA |
| siRNA 1075 | 1075 | GAUUGAAGACACAGUUGAA | 6565 | UUCAACUGUGUCUUCAAUC |
| siRNA 1076 | 1076 | AUUGAAGACACAGUUGAAA | 6566 | UUUCAACUGUGUCUUCAAU |
| siRNA 1077 | 1077 | UUGAAGACACAGUUGAAAU | 6567 | AUUUCAACUGUGUCUUCAA |
| siRNA 1078 | 1078 | UGAAGACACAGUUGAAAUG | 6568 | CAUUUCAACUGUGUCUUCA |
| siRNA 1079 | 1079 | GAAGACACAGUUGAAAUGA | 6569 | UCAUUUCAACUGUGUCUUC |
| siRNA 1080 | 1080 | AAGACACAGUUGAAAUGAC | 6570 | GUCAUUUCAACUGUGUCUU |
| siRNA 1081 | 1081 | AGACACAGUUGAAAUGACU | 6571 | AGUCAUUUCAACUGUGUCU |
| siRNA 1082 | 1082 | GACACAGUUGAAAUGACUG | 6572 | CAGUCAUUUCAACUGUGUC |
| siRNA 1083 | 1083 | ACACAGUUGAAAUGACUGA | 6573 | UCAGUCAUUUCAACUGUGU |
| siRNA 1084 | 1084 | CACAGUUGAAAUGACUGAU | 6574 | AUCAGUCAUUUCAACUGUG |
| siRNA 1085 | 1085 | ACAGUUGAAAUGACUGAUG | 6575 | CAUCAGUCAUUUCAACUGU |
| siRNA 1086 | 1086 | CAGUUGAAAUGACUGAUGA | 6576 | UCAUCAGUCAUUUCAACUG |
| siRNA 1087 | 1087 | AGUUGAAAUGACUGAUGAA | 6577 | UUCAUCAGUCAUUUCAACU |
| siRNA 1088 | 1088 | GUUGAAAUGACUGAUGAAA | 6578 | UUUCAUCAGUCAUUUCAAC |
| siRNA 1089 | 1089 | UUGAAAUGACUGAUGAAAG | 6579 | CUUUCAUCAGUCAUUUCAA |
| siRNA 1090 | 1090 | UGAAAUGACUGAUGAAAGC | 6580 | GCUUUCAUCAGUCAUUUCA |
| siRNA 1091 | 1091 | GAAAUGACUGAUGAAAGCA | 6581 | UGCUUUCAUCAGUCAUUUC |
| siRNA 1092 | 1092 | AAAUGACUGAUGAAAGCAG | 6582 | CUGCUUUCAUCAGUCAUUU |
| siRNA 1093 | 1093 | AAUGACUGAUGAAAGCAGU | 6583 | ACUGCUUUCAUCAGUCAUU |
| siRNA 1094 | 1094 | AUGACUGAUGAAAGCAGUC | 6584 | GACUGCUUUCAUCAGUCAU |
| siRNA 1095 | 1095 | UGACUGAUGAAAGCAGUCC | 6585 | GGACUGCUUUCAUCAGUCA |
| siRNA 1096 | 1096 | GACUGAUGAAAGCAGUCCU | 6586 | AGGACUGCUUUCAUCAGUC |
| siRNA 1097 | 1097 | ACUGAUGAAAGCAGUCCUC | 6587 | GAGGACUGCUUUCAUCAGU |
| siRNA 1098 | 1098 | CUGAUGAAAGCAGUCCUCA | 6588 | UGAGGACUGCUUUCAUCAG |
| siRNA 1099 | 1099 | UGAUGAAAGCAGUCCUCAU | 6589 | AUGAGGACUGCUUUCAUCA |
| siRNA 1100 | 1100 | GAUGAAAGCAGUCCUCAUC | 6590 | GAUGAGGACUGCUUUCAUC |
| siRNA 1101 | 1101 | AUGAAAGCAGUCCUCAUCC | 6591 | GGAUGAGGACUGCUUUCAU |
| siRNA 1102 | 1102 | UGAAAGCAGUCCUCAUCCC | 6592 | GGGAUGAGGACUGCUUUCA |
| siRNA 1103 | 1103 | GAAAGCAGUCCUCAUCCCU | 6593 | AGGGAUGAGGACUGCUUUC |
| siRNA 1104 | 1104 | AAAGCAGUCCUCAUCCCUU | 6594 | AAGGGAUGAGGACUGCUUU |
| siRNA 1105 | 1105 | AAGCAGUCCUCAUCCCUUA | 6595 | UAAGGGAUGAGGACUGCUU |
| siRNA 1106 | 1106 | AGCAGUCCUCAUCCCUUAG | 6596 | CUAAGGGAUGAGGACUGCU |
| siRNA 1107 | 1107 | GCAGUCCUCAUCCCUUAGC | 6597 | GCUAAGGGAUGAGGACUGC |
| siRNA 1108 | 1108 | CAGUCCUCAUCCCUUAGCU | 6598 | AGCUAAGGGAUGAGGACUG |
| siRNA 1109 | 1109 | AGUCCUCAUCCCUUAGCUG | 6599 | CAGCUAAGGGAUGAGGACU |
| siRNA 1110 | 1110 | GUCCUCAUCCCUUAGCUGG | 6600 | CCAGCUAAGGGAUGAGGAC |
| siRNA 1111 | 1111 | UCCUCAUCCCUUAGCUGGC | 6601 | GCCAGCUAAGGGAUGAGGA |
| siRNA 1112 | 1112 | CCUCAUCCCUUAGCUGGCA | 6602 | UGCCAGCUAAGGGAUGAGG |
| siRNA 1113 | 1113 | CUCAUCCCUUAGCUGGCAG | 6603 | CUGCCAGCUAAGGGAUGAG |
| siRNA 1114 | 1114 | UCAUCCCUUAGCUGGCAGC | 6604 | GCUGCCAGCUAAGGGAUGA |
| siRNA 1115 | 1115 | CAUCCCUUAGCUGGCAGCU | 6605 | AGCUGCCAGCUAAGGGAUG |
| siRNA 1116 | 1116 | AUCCCUUAGCUGGCAGCUG | 6606 | CAGCUGCCAGCUAAGGGAU |
| siRNA 1117 | 1117 | UCCCUUAGCUGGCAGCUGU | 6607 | ACAGCUGCCAGCUAAGGGA |
| siRNA 1118 | 1118 | CCCUUAGCUGGCAGCUGUU | 6608 | AACAGCUGCCAGCUAAGGG |
| siRNA 1119 | 1119 | CCUUAGCUGGCAGCUGUUU | 6609 | AAACAGCUGCCAGCUAAGG |
| siRNA 1120 | 1120 | CUUAGCUGGCAGCUGUUUU | 6610 | AAAACAGCUGCCAGCUAAG |
| siRNA 1121 | 1121 | UUAGCUGGCAGCUGUUUUG | 6611 | CAAAACAGCUGCCAGCUAA |
| siRNA 1122 | 1122 | UAGCUGGCAGCUGUUUUGA | 6612 | UCAAAACAGCUGCCAGCUA |
| siRNA 1123 | 1123 | AGCUGGCAGCUGUUUUGAA | 6613 | UUCAAAACAGCUGCCAGCU |
| siRNA 1124 | 1124 | GCUGGCAGCUGUUUUGAAG | 6614 | CUUCAAAACAGCUGCCAGC |
| siRNA 1125 | 1125 | CUGGCAGCUGUUUUGAAGA | 6615 | UCUUCAAAACAGCUGCCAG |
| siRNA 1126 | 1126 | UGGCAGCUGUUUUGAAGAU | 6616 | AUCUUCAAAACAGCUGCCA |
| siRNA 1127 | 1127 | GGCAGCUGUUUUGAAGAUU | 6617 | AAUCUUCAAAACAGCUGCC |
| siRNA 1128 | 1128 | GCAGCUGUUUUGAAGAUUU | 6618 | AAAUCUUCAAAACAGCUGC |
| siRNA 1129 | 1129 | CAGCUGUUUUGAAGAUUUG | 6619 | CAAAUCUUCAAAACAGCUG |
| siRNA 1130 | 1130 | AGCUGUUUUGAAGAUUUGG | 6620 | CCAAAUCUUCAAAACAGCU |
| siRNA 1131 | 1131 | GCUGUUUUGAAGAUUUGGC | 6621 | GCCAAAUCUUCAAAACAGC |
| siRNA 1132 | 1132 | CUGUUUUGAAGAUUUGGCA | 6622 | UGCCAAAUCUUCAAAACAG |
| siRNA 1133 | 1133 | UGUUUUGAAGAUUUGGCAG | 6623 | CUGCCAAAUCUUCAAAACA |
| siRNA 1134 | 1134 | GUUUUGAAGAUUUGGCAGA | 6624 | UCUGCCAAAUCUUCAAAAC |
| siRNA 1135 | 1135 | UUUUGAAGAUUUGGCAGAA | 6625 | UUCUGCCAAAUCUUCAAAA |
| siRNA 1136 | 1136 | UUUGAAGAUUUGGCAGAAG | 6626 | CUUCUGCCAAAUCUUCAAA |
| siRNA 1137 | 1137 | UUGAAGAUUUGGCAGAAGA | 6627 | UCUUCUGCCAAAUCUUCAA |
| siRNA 1138 | 1138 | UGAAGAUUUGGCAGAAGAG | 6628 | CUCUUCUGCCAAAUCUUCA |
| siRNA 1139 | 1139 | GAAGAUUUGGCAGAAGAGC | 6629 | GCUCUUCUGCCAAAUCUUC |
| siRNA 1140 | 1140 | AAGAUUUGGCAGAAGAGCA | 6630 | UGCUCUUCUGCCAAAUCUU |
| siRNA 1141 | 1141 | AGAUUUGGCAGAAGAGCAA | 6631 | UUGCUCUUCUGCCAAAUCU |
| siRNA 1142 | 1142 | GAUUUGGCAGAAGAGCAAG | 6632 | CUUGCUCUUCUGCCAAAUC |
| siRNA 1143 | 1143 | AUUUGGCAGAAGAGCAAGC | 6633 | GCUUGCUCUUCUGCCAAAU |
| siRNA 1144 | 1144 | UUUGGCAGAAGAGCAAGCA | 6634 | UGCUUGCUCUUCUGCCAAA |
| siRNA 1145 | 1145 | UUGGCAGAAGAGCAAGCAU | 6635 | AUGCUUGCUCUUCUGCCAA |
| siRNA 1146 | 1146 | UGGCAGAAGAGCAAGCAUU | 6636 | AAUGCUUGCUCUUCUGCCA |
| siRNA 1147 | 1147 | GGCAGAAGAGCAAGCAUUU | 6637 | AAAUGCUUGCUCUUCUGCC |
| siRNA 1148 | 1148 | GCAGAAGAGCAAGCAUUUG | 6638 | CAAAUGCUUGCUCUUCUGC |
| siRNA 1149 | 1149 | CAGAAGAGCAAGCAUUUGA | 6639 | UCAAAUGCUUGCUCUUCUG |
| siRNA 1150 | 1150 | AGAAGAGCAAGCAUUUGAU | 6640 | AUCAAAUGCUUGCUCUUCU |
| siRNA 1151 | 1151 | GAAGAGCAAGCAUUUGAUC | 6641 | GAUCAAAUGCUUGCUCUUC |
| siRNA 1152 | 1152 | AAGAGCAAGCAUUUGAUCC | 6642 | GGAUCAAAUGCUUGCUCUU |
| siRNA 1153 | 1153 | AGAGCAAGCAUUUGAUCCU | 6643 | AGGAUCAAAUGCUUGCUCU |
| siRNA 1154 | 1154 | GAGCAAGCAUUUGAUCCUU | 6644 | AAGGAUCAAAUGCUUGCUC |
| siRNA 1155 | 1155 | AGCAAGCAUUUGAUCCUUA | 6645 | UAAGGAUCAAAUGCUUGCU |
| siRNA 1156 | 1156 | GCAAGCAUUUGAUCCUUAU | 6646 | AUAAGGAUCAAAUGCUUGC |
| siRNA 1157 | 1157 | CAAGCAUUUGAUCCUUAUG | 6647 | CAUAAGGAUCAAAUGCUUG |
| siRNA 1158 | 1158 | AAGCAUUUGAUCCUUAUGA | 6648 | UCAUAAGGAUCAAAUGCUU |
| siRNA 1159 | 1159 | AGCAUUUGAUCCUUAUGAA | 6649 | UUCAUAAGGAUCAAAUGCU |
| siRNA 1160 | 1160 | GCAUUUGAUCCUUAUGAAA | 6650 | UUUCAUAAGGAUCAAAUGC |
| siRNA 1161 | 1161 | CAUUUGAUCCUUAUGAAAC | 6651 | GUUUCAUAAGGAUCAAAUG |
| siRNA 1162 | 1162 | AUUUGAUCCUUAUGAAACA | 6652 | UGUUUCAUAAGGAUCAAAU |
| siRNA 1163 | 1163 | UUUGAUCCUUAUGAAACAU | 6653 | AUGUUUCAUAAGGAUCAAA |
| siRNA 1164 | 1164 | UUGAUCCUUAUGAAACAUU | 6654 | AAUGUUUCAUAAGGAUCAA |
| siRNA 1165 | 1165 | UGAUCCUUAUGAAACAUUA | 6655 | UAAUGUUUCAUAAGGAUCA |
| siRNA 1166 | 1166 | GAUCCUUAUGAAACAUUAU | 6656 | AUAAUGUUUCAUAAGGAUC |
| siRNA 1167 | 1167 | AUCCUUAUGAAACAUUAUC | 6657 | GAUAAUGUUUCAUAAGGAU |
| siRNA 1168 | 1168 | UCCUUAUGAAACAUUAUCA | 6658 | UGAUAAUGUUUCAUAAGGA |
| siRNA 1169 | 1169 | CCUUAUGAAACAUUAUCAC | 6659 | GUGAUAAUGUUUCAUAAGG |
| siRNA 1170 | 1170 | CUUAUGAAACAUUAUCACA | 6660 | UGUGAUAAUGUUUCAUAAG |
| siRNA 1171 | 1171 | UUAUGAAACAUUAUCACAG | 6661 | CUGUGAUAAUGUUUCAUAA |
| siRNA 1172 | 1172 | UAUGAAACAUUAUCACAGG | 6662 | CCUGUGAUAAUGUUUCAUA |
| siRNA 1173 | 1173 | AUGAAACAUUAUCACAGGA | 6663 | UCCUGUGAUAAUGUUUCAU |
| siRNA 1174 | 1174 | UGAAACAUUAUCACAGGAC | 6664 | GUCCUGUGAUAAUGUUUCA |
| siRNA 1175 | 1175 | GAAACAUUAUCACAGGACA | 6665 | UGUCCUGUGAUAAUGUUUC |
| siRNA 1176 | 1176 | AAACAUUAUCACAGGACAU | 6666 | AUGUCCUGUGAUAAUGUUU |
| siRNA 1177 | 1177 | AACAUUAUCACAGGACAUU | 6667 | AAUGUCCUGUGAUAAUGUU |
| siRNA 1178 | 1178 | ACAUUAUCACAGGACAUUC | 6668 | GAAUGUCCUGUGAUAAUGU |
| siRNA 1179 | 1179 | CAUUAUCACAGGACAUUCU | 6669 | AGAAUGUCCUGUGAUAAUG |
| siRNA 1180 | 1180 | AUUAUCACAGGACAUUCUU | 6670 | AAGAAUGUCCUGUGAUAAU |
| siRNA 1181 | 1181 | UUAUCACAGGACAUUCUUU | 6671 | AAAGAAUGUCCUGUGAUAA |
| siRNA 1182 | 1182 | UAUCACAGGACAUUCUUUC | 6672 | GAAAGAAUGUCCUGUGAUA |
| siRNA 1183 | 1183 | AUCACAGGACAUUCUUUCA | 6673 | UGAAAGAAUGUCCUGUGAU |
| siRNA 1184 | 1184 | UCACAGGACAUUCUUUCAC | 6674 | GUGAAAGAAUGUCCUGUGA |
| siRNA 1185 | 1185 | CACAGGACAUUCUUUCACC | 6675 | GGUGAAAGAAUGUCCUGUG |
| siRNA 1186 | 1186 | ACAGGACAUUCUUUCACCA | 6676 | UGGUGAAAGAAUGUCCUGU |
| siRNA 1187 | 1187 | CAGGACAUUCUUUCACCAG | 6677 | CUGGUGAAAGAAUGUCCUG |
| siRNA 1188 | 1188 | AGGACAUUCUUUCACCAGA | 6678 | UCUGGUGAAAGAAUGUCCU |
| siRNA 1189 | 1189 | GGACAUUCUUUCACCAGAG | 6679 | CUCUGGUGAAAGAAUGUCC |
| siRNA 1190 | 1190 | GACAUUCUUUCACCAGAGU | 6680 | ACUCUGGUGAAAGAAUGUC |
| siRNA 1191 | 1191 | ACAUUCUUUCACCAGAGUU | 6681 | AACUCUGGUGAAAGAAUGU |
| siRNA 1192 | 1192 | CAUUCUUUCACCAGAGUUU | 6682 | AAACUCUGGUGAAAGAAUG |
| siRNA 1193 | 1193 | AUUCUUUCACCAGAGUUUC | 6683 | GAAACUCUGGUGAAAGAAU |
| siRNA 1194 | 1194 | UUCUUUCACCAGAGUUUCA | 6684 | UGAAACUCUGGUGAAAGAA |
| siRNA 1195 | 1195 | UCUUUCACCAGAGUUUCAU | 6685 | AUGAAACUCUGGUGAAAGA |
| siRNA 1196 | 1196 | CUUUCACCAGAGUUUCAUG | 6686 | CAUGAAACUCUGGUGAAAG |
| siRNA 1197 | 1197 | UUUCACCAGAGUUUCAUGA | 6687 | UCAUGAAACUCUGGUGAAA |
| siRNA 1198 | 1198 | UUCACCAGAGUUUCAUGAA | 6688 | UUCAUGAAACUCUGGUGAA |
| siRNA 1199 | 1199 | UCACCAGAGUUUCAUGAAC | 6689 | GUUCAUGAAACUCUGGUGA |
| siRNA 1200 | 1200 | CACCAGAGUUUCAUGAACA | 6690 | UGUUCAUGAAACUCUGGUG |
| siRNA 1201 | 1201 | ACCAGAGUUUCAUGAACAU | 6691 | AUGUUCAUGAAACUCUGGU |
| siRNA 1202 | 1202 | CCAGAGUUUCAUGAACAUU | 6692 | AAUGUUCAUGAAACUCUGG |
| siRNA 1203 | 1203 | CAGAGUUUCAUGAACAUUU | 6693 | AAAUGUUCAUGAAACUCUG |
| siRNA 1204 | 1204 | AGAGUUUCAUGAACAUUUC | 6694 | GAAAUGUUCAUGAAACUCU |
| siRNA 1205 | 1205 | GAGUUUCAUGAACAUUUCA | 6695 | UGAAAUGUUCAUGAAACUC |
| siRNA 1206 | 1206 | AGUUUCAUGAACAUUUCAA | 6696 | UUGAAAUGUUCAUGAAACU |
| siRNA 1207 | 1207 | GUUUCAUGAACAUUUCAAU | 6697 | AUUGAAAUGUUCAUGAAAC |
| siRNA 1208 | 1208 | UUUCAUGAACAUUUCAAUA | 6698 | UAUUGAAAUGUUCAUGAAA |
| siRNA 1209 | 1209 | UUCAUGAACAUUUCAAUAA | 6699 | UUAUUGAAAUGUUCAUGAA |
| siRNA 1210 | 1210 | UCAUGAACAUUUCAAUAAA | 6700 | UUUAUUGAAAUGUUCAUGA |
| siRNA 1211 | 1211 | CAUGAACAUUUCAAUAAAU | 6701 | AUUUAUUGAAAUGUUCAUG |
| siRNA 1212 | 1212 | AUGAACAUUUCAAUAAAUU | 6702 | AAUUUAUUGAAAUGUUCAU |
| siRNA 1213 | 1213 | UGAACAUUUCAAUAAAUUG | 6703 | CAAUUUAUUGAAAUGUUCA |
| siRNA 1214 | 1214 | GAACAUUUCAAUAAAUUGA | 6704 | UCAAUUUAUUGAAAUGUUC |
| siRNA 1215 | 1215 | AACAUUUCAAUAAAUUGAU | 6705 | AUCAAUUUAUUGAAAUGUU |
| siRNA 1216 | 1216 | ACAUUUCAAUAAAUUGAUG | 6706 | CAUCAAUUUAUUGAAAUGU |
| siRNA 1217 | 1217 | CAUUUCAAUAAAUUGAUGG | 6707 | CCAUCAAUUUAUUGAAAUG |
| siRNA 1218 | 1218 | AUUUCAAUAAAUUGAUGGC | 6708 | GCCAUCAAUUUAUUGAAAU |
| siRNA 1219 | 1219 | UUUCAAUAAAUUGAUGGCC | 6709 | GGCCAUCAAUUUAUUGAAA |
| siRNA 1220 | 1220 | UUCAAUAAAUUGAUGGCCA | 6710 | UGGCCAUCAAUUUAUUGAA |
| siRNA 1221 | 1221 | UCAAUAAAUUGAUGGCCAG | 6711 | CUGGCCAUCAAUUUAUUGA |
| siRNA 1222 | 1222 | CAAUAAAUUGAUGGCCAGA | 6712 | UCUGGCCAUCAAUUUAUUG |
| siRNA 1223 | 1223 | AAUAAAUUGAUGGCCAGAC | 6713 | GUCUGGCCAUCAAUUUAUU |
| siRNA 1224 | 1224 | AUAAAUUGAUGGCCAGACC | 6714 | GGUCUGGCCAUCAAUUUAU |
| siRNA 1225 | 1225 | UAAAUUGAUGGCCAGACCU | 6715 | AGGUCUGGCCAUCAAUUUA |
| siRNA 1226 | 1226 | AAAUUGAUGGCCAGACCUG | 6716 | CAGGUCUGGCCAUCAAUUU |
| siRNA 1227 | 1227 | AAUUGAUGGCCAGACCUGC | 6717 | GCAGGUCUGGCCAUCAAUU |
| siRNA 1228 | 1228 | AUUGAUGGCCAGACCUGCA | 6718 | UGCAGGUCUGGCCAUCAAU |
| siRNA 1229 | 1229 | UUGAUGGCCAGACCUGCAG | 6719 | CUGCAGGUCUGGCCAUCAA |
| siRNA 1230 | 1230 | UGAUGGCCAGACCUGCAGU | 6720 | ACUGCAGGUCUGGCCAUCA |
| siRNA 1231 | 1231 | GAUGGCCAGACCUGCAGUU | 6721 | AACUGCAGGUCUGGCCAUC |
| siRNA 1232 | 1232 | AUGGCCAGACCUGCAGUUG | 6722 | CAACUGCAGGUCUGGCCAU |
| siRNA 1233 | 1233 | UGGCCAGACCUGCAGUUGC | 6723 | GCAACUGCAGGUCUGGCCA |
| siRNA 1234 | 1234 | GGCCAGACCUGCAGUUGCU | 6724 | AGCAACUGCAGGUCUGGCC |
| siRNA 1235 | 1235 | GCCAGACCUGCAGUUGCUC | 6725 | GAGCAACUGCAGGUCUGGC |
| siRNA 1236 | 1236 | CCAGACCUGCAGUUGCUCU | 6726 | AGAGCAACUGCAGGUCUGG |
| siRNA 1237 | 1237 | CAGACCUGCAGUUGCUCUA | 6727 | UAGAGCAACUGCAGGUCUG |
| siRNA 1238 | 1238 | AGACCUGCAGUUGCUCUAC | 6728 | GUAGAGCAACUGCAGGUCU |
| siRNA 1239 | 1239 | GACCUGCAGUUGCUCUACA | 6729 | UGUAGAGCAACUGCAGGUC |
| siRNA 1240 | 1240 | ACCUGCAGUUGCUCUACAC | 6730 | GUGUAGAGCAACUGCAGGU |
| siRNA 1241 | 1241 | CCUGCAGUUGCUCUACACU | 6731 | AGUGUAGAGCAACUGCAGG |
| siRNA 1242 | 1242 | CUGCAGUUGCUCUACACUU | 6732 | AAGUGUAGAGCAACUGCAG |
| siRNA 1243 | 1243 | UGCAGUUGCUCUACACUUU | 6733 | AAAGUGUAGAGCAACUGCA |
| siRNA 1244 | 1244 | GCAGUUGCUCUACACUUUC | 6734 | GAAAGUGUAGAGCAACUGC |
| siRNA 1245 | 1245 | CAGUUGCUCUACACUUUCA | 6735 | UGAAAGUGUAGAGCAACUG |
| siRNA 1246 | 1246 | AGUUGCUCUACACUUUCAG | 6736 | CUGAAAGUGUAGAGCAACU |
| siRNA 1247 | 1247 | GUUGCUCUACACUUUCAGU | 6737 | ACUGAAAGUGUAGAGCAAC |
| siRNA 1248 | 1248 | UUGCUCUACACUUUCAGUC | 6738 | GACUGAAAGUGUAGAGCAA |
| siRNA 1249 | 1249 | UGCUCUACACUUUCAGUCC | 6739 | GGACUGAAAGUGUAGAGCA |
| siRNA 1250 | 1250 | GCUCUACACUUUCAGUCCA | 6740 | UGGACUGAAAGUGUAGAGC |
| siRNA 1251 | 1251 | CUCUACACUUUCAGUCCAU | 6741 | AUGGACUGAAAGUGUAGAG |
| siRNA 1252 | 1252 | UCUACACUUUCAGUCCAUU | 6742 | AAUGGACUGAAAGUGUAGA |
| siRNA 1253 | 1253 | CUACACUUUCAGUCCAUUG | 6743 | CAAUGGACUGAAAGUGUAG |
| siRNA 1254 | 1254 | UACACUUUCAGUCCAUUGC | 6744 | GCAAUGGACUGAAAGUGUA |
| siRNA 1255 | 1255 | ACACUUUCAGUCCAUUGCU | 6745 | AGCAAUGGACUGAAAGUGU |
| siRNA 1256 | 1256 | CACUUUCAGUCCAUUGCUG | 6746 | CAGCAAUGGACUGAAAGUG |
| siRNA 1257 | 1257 | ACUUUCAGUCCAUUGCUGA | 6747 | UCAGCAAUGGACUGAAAGU |
| siRNA 1258 | 1258 | CUUUCAGUCCAUUGCUGAU | 6748 | AUCAGCAAUGGACUGAAAG |
| siRNA 1259 | 1259 | UUUCAGUCCAUUGCUGAUG | 6749 | CAUCAGCAAUGGACUGAAA |
| siRNA 1260 | 1260 | UUCAGUCCAUUGCUGAUGG | 6750 | CCAUCAGCAAUGGACUGAA |
| siRNA 1261 | 1261 | UCAGUCCAUUGCUGAUGGU | 6751 | ACCAUCAGCAAUGGACUGA |
| siRNA 1262 | 1262 | CAGUCCAUUGCUGAUGGUU | 6752 | AACCAUCAGCAAUGGACUG |
| siRNA 1263 | 1263 | AGUCCAUUGCUGAUGGUUU | 6753 | AAACCAUCAGCAAUGGACU |
| siRNA 1264 | 1264 | GUCCAUUGCUGAUGGUUUU | 6754 | AAAACCAUCAGCAAUGGAC |
| siRNA 1265 | 1265 | UCCAUUGCUGAUGGUUUUA | 6755 | UAAAACCAUCAGCAAUGGA |
| siRNA 1266 | 1266 | CCAUUGCUGAUGGUUUUAA | 6756 | UUAAAACCAUCAGCAAUGG |
| siRNA 1267 | 1267 | CAUUGCUGAUGGUUUUAAA | 6757 | UUUAAAACCAUCAGCAAUG |
| siRNA 1268 | 1268 | AUUGCUGAUGGUUUUAAAG | 6758 | CUUUAAAACCAUCAGCAAU |
| siRNA 1269 | 1269 | UUGCUGAUGGUUUUAAAGA | 6759 | UCUUUAAAACCAUCAGCAA |
| siRNA 1270 | 1270 | UGCUGAUGGUUUUAAAGAG | 6760 | CUCUUUAAAACCAUCAGCA |
| siRNA 1271 | 1271 | GCUGAUGGUUUUAAAGAGG | 6761 | CCUCUUUAAAACCAUCAGC |
| siRNA 1272 | 1272 | CUGAUGGUUUUAAAGAGGC | 6762 | GCCUCUUUAAAACCAUCAG |
| siRNA 1273 | 1273 | UGAUGGUUUUAAAGAGGCA | 6763 | UGCCUCUUUAAAACCAUCA |
| siRNA 1274 | 1274 | GAUGGUUUUAAAGAGGCAG | 6764 | CUGCCUCUUUAAAACCAUC |
| siRNA 1275 | 1275 | AUGGUUUUAAAGAGGCAGU | 6765 | ACUGCCUCUUUAAAACCAU |
| siRNA 1276 | 1276 | UGGUUUUAAAGAGGCAGUU | 6766 | AACUGCCUCUUUAAAACCA |
| siRNA 1277 | 1277 | GGUUUUAAAGAGGCAGUUC | 6767 | GAACUGCCUCUUUAAAACC |
| siRNA 1278 | 1278 | GUUUUAAAGAGGCAGUUCG | 6768 | CGAACUGCCUCUUUAAAAC |
| siRNA 1279 | 1279 | UUUUAAAGAGGCAGUUCGU | 6769 | ACGAACUGCCUCUUUAAAA |
| siRNA 1280 | 1280 | UUUAAAGAGGCAGUUCGUU | 6770 | AACGAACUGCCUCUUUAAA |
| siRNA 1281 | 1281 | UUAAAGAGGCAGUUCGUUA | 6771 | UAACGAACUGCCUCUUUAA |
| siRNA 1282 | 1282 | UAAAGAGGCAGUUCGUUAU | 6772 | AUAACGAACUGCCUCUUUA |
| siRNA 1283 | 1283 | AAAGAGGCAGUUCGUUAUG | 6773 | CAUAACGAACUGCCUCUUU |
| siRNA 1284 | 1284 | AAGAGGCAGUUCGUUAUGU | 6774 | ACAUAACGAACUGCCUCUU |
| siRNA 1285 | 1285 | AGAGGCAGUUCGUUAUGUC | 6775 | GACAUAACGAACUGCCUCU |
| siRNA 1286 | 1286 | GAGGCAGUUCGUUAUGUCC | 6776 | GGACAUAACGAACUGCCUC |
| siRNA 1287 | 1287 | AGGCAGUUCGUUAUGUCCU | 6777 | AGGACAUAACGAACUGCCU |
| siRNA 1288 | 1288 | GGCAGUUCGUUAUGUCCUU | 6778 | AAGGACAUAACGAACUGCC |
| siRNA 1289 | 1289 | GCAGUUCGUUAUGUCCUUC | 6779 | GAAGGACAUAACGAACUGC |
| siRNA 1290 | 1290 | CAGUUCGUUAUGUCCUUCC | 6780 | GGAAGGACAUAACGAACUG |
| siRNA 1291 | 1291 | AGUUCGUUAUGUCCUUCCA | 6781 | UGGAAGGACAUAACGAACU |
| siRNA 1292 | 1292 | GUUCGUUAUGUCCUUCCAC | 6782 | GUGGAAGGACAUAACGAAC |
| siRNA 1293 | 1293 | UUCGUUAUGUCCUUCCACG | 6783 | CGUGGAAGGACAUAACGAA |
| siRNA 1294 | 1294 | UCGUUAUGUCCUUCCACGU | 6784 | ACGUGGAAGGACAUAACGA |
| siRNA 1295 | 1295 | CGUUAUGUCCUUCCACGUC | 6785 | GACGUGGAAGGACAUAACG |
| siRNA 1296 | 1296 | GUUAUGUCCUUCCACGUCU | 6786 | AGACGUGGAAGGACAUAAC |
| siRNA 1297 | 1297 | UUAUGUCCUUCCACGUCUU | 6787 | AAGACGUGGAAGGACAUAA |
| siRNA 1298 | 1298 | UAUGUCCUUCCACGUCUUA | 6788 | UAAGACGUGGAAGGACAUA |
| siRNA 1299 | 1299 | AUGUCCUUCCACGUCUUAU | 6789 | AUAAGACGUGGAAGGACAU |
| siRNA 1300 | 1300 | UGUCCUUCCACGUCUUAUG | 6790 | CAUAAGACGUGGAAGGACA |
| siRNA 1301 | 1301 | GUCCUUCCACGUCUUAUGC | 6791 | GCAUAAGACGUGGAAGGAC |
| siRNA 1302 | 1302 | UCCUUCCACGUCUUAUGCU | 6792 | AGCAUAAGACGUGGAAGGA |
| siRNA 1303 | 1303 | CCUUCCACGUCUUAUGCUG | 6793 | CAGCAUAAGACGUGGAAGG |
| siRNA 1304 | 1304 | CUUCCACGUCUUAUGCUGG | 6794 | CCAGCAUAAGACGUGGAAG |
| siRNA 1305 | 1305 | UUCCACGUCUUAUGCUGGU | 6795 | ACCAGCAUAAGACGUGGAA |
| siRNA 1306 | 1306 | UCCACGUCUUAUGCUGGUG | 6796 | CACCAGCAUAAGACGUGGA |
| siRNA 1307 | 1307 | CCACGUCUUAUGCUGGUGC | 6797 | GCACCAGCAUAAGACGUGG |
| siRNA 1308 | 1308 | CACGUCUUAUGCUGGUGCC | 6798 | GGCACCAGCAUAAGACGUG |
| siRNA 1309 | 1309 | ACGUCUUAUGCUGGUGCCA | 6799 | UGGCACCAGCAUAAGACGU |
| siRNA 1310 | 1310 | CGUCUUAUGCUGGUGCCAG | 6800 | CUGGCACCAGCAUAAGACG |
| siRNA 1311 | 1311 | GUCUUAUGCUGGUGCCAGU | 6801 | ACUGGCACCAGCAUAAGAC |
| siRNA 1312 | 1312 | UCUUAUGCUGGUGCCAGUG | 6802 | CACUGGCACCAGCAUAAGA |
| siRNA 1313 | 1313 | CUUAUGCUGGUGCCAGUGU | 6803 | ACACUGGCACCAGCAUAAG |
| siRNA 1314 | 1314 | UUAUGCUGGUGCCAGUGUA | 6804 | UACACUGGCACCAGCAUAA |
| siRNA 1315 | 1315 | UAUGCUGGUGCCAGUGUAU | 6805 | AUACACUGGCACCAGCAUA |
| siRNA 1316 | 1316 | AUGCUGGUGCCAGUGUAUC | 6806 | GAUACACUGGCACCAGCAU |
| siRNA 1317 | 1317 | UGCUGGUGCCAGUGUAUCA | 6807 | UGAUACACUGGCACCAGCA |
| siRNA 1318 | 1318 | GCUGGUGCCAGUGUAUCAC | 6808 | GUGAUACACUGGCACCAGC |
| siRNA 1319 | 1319 | CUGGUGCCAGUGUAUCACU | 6809 | AGUGAUACACUGGCACCAG |
| siRNA 1320 | 1320 | UGGUGCCAGUGUAUCACUG | 6810 | CAGUGAUACACUGGCACCA |
| siRNA 1321 | 1321 | GGUGCCAGUGUAUCACUGU | 6811 | ACAGUGAUACACUGGCACC |
| siRNA 1322 | 1322 | GUGCCAGUGUAUCACUGUU | 6812 | AACAGUGAUACACUGGCAC |
| siRNA 1323 | 1323 | UGCCAGUGUAUCACUGUUG | 6813 | CAACAGUGAUACACUGGCA |
| siRNA 1324 | 1324 | GCCAGUGUAUCACUGUUGG | 6814 | CCAACAGUGAUACACUGGC |
| siRNA 1325 | 1325 | CCAGUGUAUCACUGUUGGC | 6815 | GCCAACAGUGAUACACUGG |
| siRNA 1326 | 1326 | CAGUGUAUCACUGUUGGCA | 6816 | UGCCAACAGUGAUACACUG |
| siRNA 1327 | 1327 | AGUGUAUCACUGUUGGCAC | 6817 | GUGCCAACAGUGAUACACU |
| siRNA 1328 | 1328 | GUGUAUCACUGUUGGCACU | 6818 | AGUGCCAACAGUGAUACAC |
| siRNA 1329 | 1329 | UGUAUCACUGUUGGCACUA | 6819 | UAGUGCCAACAGUGAUACA |
| siRNA 1330 | 1330 | GUAUCACUGUUGGCACUAC | 6820 | GUAGUGCCAACAGUGAUAC |
| siRNA 1331 | 1331 | UAUCACUGUUGGCACUACU | 6821 | AGUAGUGCCAACAGUGAUA |
| siRNA 1332 | 1332 | AUCACUGUUGGCACUACUU | 6822 | AAGUAGUGCCAACAGUGAU |
| siRNA 1333 | 1333 | UCACUGUUGGCACUACUUU | 6823 | AAAGUAGUGCCAACAGUGA |
| siRNA 1334 | 1334 | CACUGUUGGCACUACUUUG | 6824 | CAAAGUAGUGCCAACAGUG |
| siRNA 1335 | 1335 | ACUGUUGGCACUACUUUGA | 6825 | UCAAAGUAGUGCCAACAGU |
| siRNA 1336 | 1336 | CUGUUGGCACUACUUUGAG | 6826 | CUCAAAGUAGUGCCAACAG |
| siRNA 1337 | 1337 | UGUUGGCACUACUUUGAGU | 6827 | ACUCAAAGUAGUGCCAACA |
| siRNA 1338 | 1338 | GUUGGCACUACUUUGAGUU | 6828 | AACUCAAAGUAGUGCCAAC |
| siRNA 1339 | 1339 | UUGGCACUACUUUGAGUUA | 6829 | UAACUCAAAGUAGUGCCAA |
| siRNA 1340 | 1340 | UGGCACUACUUUGAGUUAC | 6830 | GUAACUCAAAGUAGUGCCA |
| siRNA 1341 | 1341 | GGCACUACUUUGAGUUACU | 6831 | AGUAACUCAAAGUAGUGCC |
| siRNA 1342 | 1342 | GCACUACUUUGAGUUACUA | 6832 | UAGUAACUCAAAGUAGUGC |
| siRNA 1343 | 1343 | CACUACUUUGAGUUACUAA | 6833 | UUAGUAACUCAAAGUAGUG |
| siRNA 1344 | 1344 | ACUACUUUGAGUUACUAAA | 6834 | UUUAGUAACUCAAAGUAGU |
| siRNA 1345 | 1345 | CUACUUUGAGUUACUAAAG | 6835 | CUUUAGUAACUCAAAGUAG |
| siRNA 1346 | 1346 | UACUUUGAGUUACUAAAGC | 6836 | GCUUUAGUAACUCAAAGUA |
| siRNA 1347 | 1347 | ACUUUGAGUUACUAAAGCA | 6837 | UGCUUUAGUAACUCAAAGU |
| siRNA 1348 | 1348 | CUUUGAGUUACUAAAGCAA | 6838 | UUGCUUUAGUAACUCAAAG |
| siRNA 1349 | 1349 | UUUGAGUUACUAAAGCAAU | 6839 | AUUGCUUUAGUAACUCAAA |
| siRNA 1350 | 1350 | UUGAGUUACUAAAGCAAUU | 6840 | AAUUGCUUUAGUAACUCAA |
| siRNA 1351 | 1351 | UGAGUUACUAAAGCAAUUG | 6841 | CAAUUGCUUUAGUAACUCA |
| siRNA 1352 | 1352 | GAGUUACUAAAGCAAUUGA | 6842 | UCAAUUGCUUUAGUAACUC |
| siRNA 1353 | 1353 | AGUUACUAAAGCAAUUGAA | 6843 | UUCAAUUGCUUUAGUAACU |
| siRNA 1354 | 1354 | GUUACUAAAGCAAUUGAAA | 6844 | UUUCAAUUGCUUUAGUAAC |
| siRNA 1355 | 1355 | UUACUAAAGCAAUUGAAAG | 6845 | CUUUCAAUUGCUUUAGUAA |
| siRNA 1356 | 1356 | UACUAAAGCAAUUGAAAGC | 6846 | GCUUUCAAUUGCUUUAGUA |
| siRNA 1357 | 1357 | ACUAAAGCAAUUGAAAGCA | 6847 | UGCUUUCAAUUGCUUUAGU |
| siRNA 1358 | 1358 | CUAAAGCAAUUGAAAGCAU | 6848 | AUGCUUUCAAUUGCUUUAG |
| siRNA 1359 | 1359 | UAAAGCAAUUGAAAGCAUG | 6849 | CAUGCUUUCAAUUGCUUUA |
| siRNA 1360 | 1360 | AAAGCAAUUGAAAGCAUGU | 6850 | ACAUGCUUUCAAUUGCUUU |
| siRNA 1361 | 1361 | AAGCAAUUGAAAGCAUGUA | 6851 | UACAUGCUUUCAAUUGCUU |
| siRNA 1362 | 1362 | AGCAAUUGAAAGCAUGUAG | 6852 | CUACAUGCUUUCAAUUGCU |
| siRNA 1363 | 1363 | GCAAUUGAAAGCAUGUAGU | 6853 | ACUACAUGCUUUCAAUUGC |
| siRNA 1364 | 1364 | CAAUUGAAAGCAUGUAGUG | 6854 | CACUACAUGCUUUCAAUUG |
| siRNA 1365 | 1365 | AAUUGAAAGCAUGUAGUGA | 6855 | UCACUACAUGCUUUCAAUU |
| siRNA 1366 | 1366 | AUUGAAAGCAUGUAGUGAA | 6856 | UUCACUACAUGCUUUCAAU |
| siRNA 1367 | 1367 | UUGAAAGCAUGUAGUGAAG | 6857 | CUUCACUACAUGCUUUCAA |
| siRNA 1368 | 1368 | UGAAAGCAUGUAGUGAAGA | 6858 | UCUUCACUACAUGCUUUCA |
| siRNA 1369 | 1369 | GAAAGCAUGUAGUGAAGAA | 6859 | UUCUUCACUACAUGCUUUC |
| siRNA 1370 | 1370 | AAAGCAUGUAGUGAAGAAC | 6860 | GUUCUUCACUACAUGCUUU |
| siRNA 1371 | 1371 | AAGCAUGUAGUGAAGAACA | 6861 | UGUUCUUCACUACAUGCUU |
| siRNA 1372 | 1372 | AGCAUGUAGUGAAGAACAA | 6862 | UUGUUCUUCACUACAUGCU |
| siRNA 1373 | 1373 | GCAUGUAGUGAAGAACAAG | 6863 | CUUGUUCUUCACUACAUGC |
| siRNA 1374 | 1374 | CAUGUAGUGAAGAACAAGA | 6864 | UCUUGUUCUUCACUACAUG |
| siRNA 1375 | 1375 | AUGUAGUGAAGAACAAGAA | 6865 | UUCUUGUUCUUCACUACAU |
| siRNA 1376 | 1376 | UGUAGUGAAGAACAAGAAG | 6866 | CUUCUUGUUCUUCACUACA |
| siRNA 1377 | 1377 | GUAGUGAAGAACAAGAAGA | 6867 | UCUUCUUGUUCUUCACUAC |
| siRNA 1378 | 1378 | UAGUGAAGAACAAGAAGAC | 6868 | GUCUUCUUGUUCUUCACUA |
| siRNA 1379 | 1379 | AGUGAAGAACAAGAAGACA | 6869 | UGUCUUCUUGUUCUUCACU |
| siRNA 1380 | 1380 | GUGAAGAACAAGAAGACAG | 6870 | CUGUCUUCUUGUUCUUCAC |
| siRNA 1381 | 1381 | UGAAGAACAAGAAGACAGA | 6871 | UCUGUCUUCUUGUUCUUCA |
| siRNA 1382 | 1382 | GAAGAACAAGAAGACAGAG | 6872 | CUCUGUCUUCUUGUUCUUC |
| siRNA 1383 | 1383 | AAGAACAAGAAGACAGAGA | 6873 | UCUCUGUCUUCUUGUUCUU |
| siRNA 1384 | 1384 | AGAACAAGAAGACAGAGAA | 6874 | UUCUCUGUCUUCUUGUUCU |
| siRNA 1385 | 1385 | GAACAAGAAGACAGAGAAU | 6875 | AUUCUCUGUCUUCUUGUUC |
| siRNA 1386 | 1386 | AACAAGAAGACAGAGAAUG | 6876 | CAUUCUCUGUCUUCUUGUU |
| siRNA 1387 | 1387 | ACAAGAAGACAGAGAAUGU | 6877 | ACAUUCUCUGUCUUCUUGU |
| siRNA 1388 | 1388 | CAAGAAGACAGAGAAUGUU | 6878 | AACAUUCUCUGUCUUCUUG |
| siRNA 1389 | 1389 | AAGAAGACAGAGAAUGUUU | 6879 | AAACAUUCUCUGUCUUCUU |
| siRNA 1390 | 1390 | AGAAGACAGAGAAUGUUUG | 6880 | CAAACAUUCUCUGUCUUCU |
| siRNA 1391 | 1391 | GAAGACAGAGAAUGUUUGA | 6881 | UCAAACAUUCUCUGUCUUC |
| siRNA 1392 | 1392 | AAGACAGAGAAUGUUUGAA | 6882 | UUCAAACAUUCUCUGUCUU |
| siRNA 1393 | 1393 | AGACAGAGAAUGUUUGAAC | 6883 | GUUCAAACAUUCUCUGUCU |
| siRNA 1394 | 1394 | GACAGAGAAUGUUUGAACC | 6884 | GGUUCAAACAUUCUCUGUC |
| siRNA 1395 | 1395 | ACAGAGAAUGUUUGAACCA | 6885 | UGGUUCAAACAUUCUCUGU |
| siRNA 1396 | 1396 | CAGAGAAUGUUUGAACCAA | 6886 | UUGGUUCAAACAUUCUCUG |
| siRNA 1397 | 1397 | AGAGAAUGUUUGAACCAAG | 6887 | CUUGGUUCAAACAUUCUCU |
| siRNA 1398 | 1398 | GAGAAUGUUUGAACCAAGC | 6888 | GCUUGGUUCAAACAUUCUC |
| siRNA 1399 | 1399 | AGAAUGUUUGAACCAAGCU | 6889 | AGCUUGGUUCAAACAUUCU |
| siRNA 1400 | 1400 | GAAUGUUUGAACCAAGCUA | 6890 | UAGCUUGGUUCAAACAUUC |
| siRNA 1401 | 1401 | AAUGUUUGAACCAAGCUAU | 6891 | AUAGCUUGGUUCAAACAUU |
| siRNA 1402 | 1402 | AUGUUUGAACCAAGCUAUU | 6892 | AAUAGCUUGGUUCAAACAU |
| siRNA 1403 | 1403 | UGUUUGAACCAAGCUAUUA | 6893 | UAAUAGCUUGGUUCAAACA |
| siRNA 1404 | 1404 | GUUUGAACCAAGCUAUUAC | 6894 | GUAAUAGCUUGGUUCAAAC |
| siRNA 1405 | 1405 | UUUGAACCAAGCUAUUACU | 6895 | AGUAAUAGCUUGGUUCAAA |
| siRNA 1406 | 1406 | UUGAACCAAGCUAUUACUG | 6896 | CAGUAAUAGCUUGGUUCAA |
| siRNA 1407 | 1407 | UGAACCAAGCUAUUACUGC | 6897 | GCAGUAAUAGCUUGGUUCA |
| siRNA 1408 | 1408 | GAACCAAGCUAUUACUGCU | 6898 | AGCAGUAAUAGCUUGGUUC |
| siRNA 1409 | 1409 | AACCAAGCUAUUACUGCUC | 6899 | GAGCAGUAAUAGCUUGGUU |
| siRNA 1410 | 1410 | ACCAAGCUAUUACUGCUCU | 6900 | AGAGCAGUAAUAGCUUGGU |
| siRNA 1411 | 1411 | CCAAGCUAUUACUGCUCUC | 6901 | GAGAGCAGUAAUAGCUUGG |
| siRNA 1412 | 1412 | CAAGCUAUUACUGCUCUCA | 6902 | UGAGAGCAGUAAUAGCUUG |
| siRNA 1413 | 1413 | AAGCUAUUACUGCUCUCAU | 6903 | AUGAGAGCAGUAAUAGCUU |
| siRNA 1414 | 1414 | AGCUAUUACUGCUCUCAUG | 6904 | CAUGAGAGCAGUAAUAGCU |
| siRNA 1415 | 1415 | GCUAUUACUGCUCUCAUGA | 6905 | UCAUGAGAGCAGUAAUAGC |
| siRNA 1416 | 1416 | CUAUUACUGCUCUCAUGAA | 6906 | UUCAUGAGAGCAGUAAUAG |
| siRNA 1417 | 1417 | UAUUACUGCUCUCAUGAAU | 6907 | AUUCAUGAGAGCAGUAAUA |
| siRNA 1418 | 1418 | AUUACUGCUCUCAUGAAUC | 6908 | GAUUCAUGAGAGCAGUAAU |
| siRNA 1419 | 1419 | UUACUGCUCUCAUGAAUCU | 6909 | AGAUUCAUGAGAGCAGUAA |
| siRNA 1420 | 1420 | UACUGCUCUCAUGAAUCUC | 6910 | GAGAUUCAUGAGAGCAGUA |
| siRNA 1421 | 1421 | ACUGCUCUCAUGAAUCUCC | 6911 | GGAGAUUCAUGAGAGCAGU |
| siRNA 1422 | 1422 | CUGCUCUCAUGAAUCUCCA | 6912 | UGGAGAUUCAUGAGAGCAG |
| siRNA 1423 | 1423 | UGCUCUCAUGAAUCUCCAA | 6913 | UUGGAGAUUCAUGAGAGCA |
| siRNA 1424 | 1424 | GCUCUCAUGAAUCUCCAAG | 6914 | CUUGGAGAUUCAUGAGAGC |
| siRNA 1425 | 1425 | CUCUCAUGAAUCUCCAAGG | 6915 | CCUUGGAGAUUCAUGAGAG |
| siRNA 1426 | 1426 | UCUCAUGAAUCUCCAAGGU | 6916 | ACCUUGGAGAUUCAUGAGA |
| siRNA 1427 | 1427 | CUCAUGAAUCUCCAAGGUA | 6917 | UACCUUGGAGAUUCAUGAG |
| siRNA 1428 | 1428 | UCAUGAAUCUCCAAGGUAG | 6918 | CUACCUUGGAGAUUCAUGA |
| siRNA 1429 | 1429 | CAUGAAUCUCCAAGGUAGC | 6919 | GCUACCUUGGAGAUUCAUG |
| siRNA 1430 | 1430 | AUGAAUCUCCAAGGUAGCA | 6920 | UGCUACCUUGGAGAUUCAU |
| siRNA 1431 | 1431 | UGAAUCUCCAAGGUAGCAU | 6921 | AUGCUACCUUGGAGAUUCA |
| siRNA 1432 | 1432 | GAAUCUCCAAGGUAGCAUG | 6922 | CAUGCUACCUUGGAGAUUC |
| siRNA 1433 | 1433 | AAUCUCCAAGGUAGCAUGG | 6923 | CCAUGCUACCUUGGAGAUU |
| siRNA 1434 | 1434 | AUCUCCAAGGUAGCAUGGA | 6924 | UCCAUGCUACCUUGGAGAU |
| siRNA 1435 | 1435 | UCUCCAAGGUAGCAUGGAC | 6925 | GUCCAUGCUACCUUGGAGA |
| siRNA 1436 | 1436 | CUCCAAGGUAGCAUGGACC | 6926 | GGUCCAUGCUACCUUGGAG |
| siRNA 1437 | 1437 | UCCAAGGUAGCAUGGACCG | 6927 | CGGUCCAUGCUACCUUGGA |
| siRNA 1438 | 1438 | CCAAGGUAGCAUGGACCGA | 6928 | UCGGUCCAUGCUACCUUGG |
| siRNA 1439 | 1439 | CAAGGUAGCAUGGACCGAA | 6929 | UUCGGUCCAUGCUACCUUG |
| siRNA 1440 | 1440 | AAGGUAGCAUGGACCGAAU | 6930 | AUUCGGUCCAUGCUACCUU |
| siRNA 1441 | 1441 | AGGUAGCAUGGACCGAAUU | 6931 | AAUUCGGUCCAUGCUACCU |
| siRNA 1442 | 1442 | GGUAGCAUGGACCGAAUUU | 6932 | AAAUUCGGUCCAUGCUACC |
| siRNA 1443 | 1443 | GUAGCAUGGACCGAAUUUA | 6933 | UAAAUUCGGUCCAUGCUAC |
| siRNA 1444 | 1444 | UAGCAUGGACCGAAUUUAC | 6934 | GUAAAUUCGGUCCAUGCUA |
| siRNA 1445 | 1445 | AGCAUGGACCGAAUUUACA | 6935 | UGUAAAUUCGGUCCAUGCU |
| siRNA 1446 | 1446 | GCAUGGACCGAAUUUACAA | 6936 | UUGUAAAUUCGGUCCAUGC |
| siRNA 1447 | 1447 | CAUGGACCGAAUUUACAAG | 6937 | CUUGUAAAUUCGGUCCAUG |
| siRNA 1448 | 1448 | AUGGACCGAAUUUACAAGC | 6938 | GCUUGUAAAUUCGGUCCAU |
| siRNA 1449 | 1449 | UGGACCGAAUUUACAAGCA | 6939 | UGCUUGUAAAUUCGGUCCA |
| siRNA 1450 | 1450 | GGACCGAAUUUACAAGCAG | 6940 | CUGCUUGUAAAUUCGGUCC |
| siRNA 1451 | 1451 | GACCGAAUUUACAAGCAGU | 6941 | ACUGCUUGUAAAUUCGGUC |
| siRNA 1452 | 1452 | ACCGAAUUUACAAGCAGUA | 6942 | UACUGCUUGUAAAUUCGGU |
| siRNA 1453 | 1453 | CCGAAUUUACAAGCAGUAU | 6943 | AUACUGCUUGUAAAUUCGG |
| siRNA 1454 | 1454 | CGAAUUUACAAGCAGUAUU | 6944 | AAUACUGCUUGUAAAUUCG |
| siRNA 1455 | 1455 | GAAUUUACAAGCAGUAUUC | 6945 | GAAUACUGCUUGUAAAUUC |
| siRNA 1456 | 1456 | AAUUUACAAGCAGUAUUCA | 6946 | UGAAUACUGCUUGUAAAUU |
| siRNA 1457 | 1457 | AUUUACAAGCAGUAUUCAC | 6947 | GUGAAUACUGCUUGUAAAU |
| siRNA 1458 | 1458 | UUUACAAGCAGUAUUCACC | 6948 | GGUGAAUACUGCUUGUAAA |
| siRNA 1459 | 1459 | UUACAAGCAGUAUUCACCU | 6949 | AGGUGAAUACUGCUUGUAA |
| siRNA 1460 | 1460 | UACAAGCAGUAUUCACCUA | 6950 | UAGGUGAAUACUGCUUGUA |
| siRNA 1461 | 1461 | ACAAGCAGUAUUCACCUAG | 6951 | CUAGGUGAAUACUGCUUGU |
| siRNA 1462 | 1462 | CAAGCAGUAUUCACCUAGA | 6952 | UCUAGGUGAAUACUGCUUG |
| siRNA 1463 | 1463 | AAGCAGUAUUCACCUAGAC | 6953 | GUCUAGGUGAAUACUGCUU |
| siRNA 1464 | 1464 | AGCAGUAUUCACCUAGACG | 6954 | CGUCUAGGUGAAUACUGCU |
| siRNA 1465 | 1465 | GCAGUAUUCACCUAGACGU | 6955 | ACGUCUAGGUGAAUACUGC |
| siRNA 1466 | 1466 | CAGUAUUCACCUAGACGUC | 6956 | GACGUCUAGGUGAAUACUG |
| siRNA 1467 | 1467 | AGUAUUCACCUAGACGUCG | 6957 | CGACGUCUAGGUGAAUACU |
| siRNA 1468 | 1468 | GUAUUCACCUAGACGUCGA | 6958 | UCGACGUCUAGGUGAAUAC |
| siRNA 1469 | 1469 | UAUUCACCUAGACGUCGAC | 6959 | GUCGACGUCUAGGUGAAUA |
| siRNA 1470 | 1470 | AUUCACCUAGACGUCGACC | 6960 | GGUCGACGUCUAGGUGAAU |
| siRNA 1471 | 1471 | UUCACCUAGACGUCGACCU | 6961 | AGGUCGACGUCUAGGUGAA |
| siRNA 1472 | 1472 | UCACCUAGACGUCGACCUG | 6962 | CAGGUCGACGUCUAGGUGA |
| siRNA 1473 | 1473 | CACCUAGACGUCGACCUGG | 6963 | CCAGGUCGACGUCUAGGUG |
| siRNA 1474 | 1474 | ACCUAGACGUCGACCUGGA | 6964 | UCCAGGUCGACGUCUAGGU |
| siRNA 1475 | 1475 | CCUAGACGUCGACCUGGAG | 6965 | CUCCAGGUCGACGUCUAGG |
| siRNA 1476 | 1476 | CUAGACGUCGACCUGGAGA | 6966 | UCUCCAGGUCGACGUCUAG |
| siRNA 1477 | 1477 | UAGACGUCGACCUGGAGAU | 6967 | AUCUCCAGGUCGACGUCUA |
| siRNA 1478 | 1478 | AGACGUCGACCUGGAGAUC | 6968 | GAUCUCCAGGUCGACGUCU |
| siRNA 1479 | 1479 | GACGUCGACCUGGAGAUCC | 6969 | GGAUCUCCAGGUCGACGUC |
| siRNA 1480 | 1480 | ACGUCGACCUGGAGAUCCU | 6970 | AGGAUCUCCAGGUCGACGU |
| siRNA 1481 | 1481 | CGUCGACCUGGAGAUCCUG | 6971 | CAGGAUCUCCAGGUCGACG |
| siRNA 1482 | 1482 | GUCGACCUGGAGAUCCUGU | 6972 | ACAGGAUCUCCAGGUCGAC |
| siRNA 1483 | 1483 | UCGACCUGGAGAUCCUGUU | 6973 | AACAGGAUCUCCAGGUCGA |
| siRNA 1484 | 1484 | CGACCUGGAGAUCCUGUUU | 6974 | AAACAGGAUCUCCAGGUCG |
| siRNA 1485 | 1485 | GACCUGGAGAUCCUGUUUG | 6975 | CAAACAGGAUCUCCAGGUC |
| siRNA 1486 | 1486 | ACCUGGAGAUCCUGUUUGC | 6976 | GCAAACAGGAUCUCCAGGU |
| siRNA 1487 | 1487 | CCUGGAGAUCCUGUUUGCC | 6977 | GGCAAACAGGAUCUCCAGG |
| siRNA 1488 | 1488 | CUGGAGAUCCUGUUUGCCC | 6978 | GGGCAAACAGGAUCUCCAG |
| siRNA 1489 | 1489 | UGGAGAUCCUGUUUGCCCU | 6979 | AGGGCAAACAGGAUCUCCA |
| siRNA 1490 | 1490 | GGAGAUCCUGUUUGCCCUU | 6980 | AAGGGCAAACAGGAUCUCC |
| siRNA 1491 | 1491 | GAGAUCCUGUUUGCCCUUU | 6981 | AAAGGGCAAACAGGAUCUC |
| siRNA 1492 | 1492 | AGAUCCUGUUUGCCCUUUU | 6982 | AAAAGGGCAAACAGGAUCU |
| siRNA 1493 | 1493 | GAUCCUGUUUGCCCUUUUU | 6983 | AAAAAGGGCAAACAGGAUC |
| siRNA 1494 | 1494 | AUCCUGUUUGCCCUUUUUA | 6984 | UAAAAAGGGCAAACAGGAU |
| siRNA 1495 | 1495 | UCCUGUUUGCCCUUUUUAU | 6985 | AUAAAAAGGGCAAACAGGA |
| siRNA 1496 | 1496 | CCUGUUUGCCCUUUUUAUA | 6986 | UAUAAAAAGGGCAAACAGG |
| siRNA 1497 | 1497 | CUGUUUGCCCUUUUUAUAG | 6987 | CUAUAAAAAGGGCAAACAG |
| siRNA 1498 | 1498 | UGUUUGCCCUUUUUAUAGU | 6988 | ACUAUAAAAAGGGCAAACA |
| siRNA 1499 | 1499 | GUUUGCCCUUUUUAUAGUC | 6989 | GACUAUAAAAAGGGCAAAC |
| siRNA 1500 | 1500 | UUUGCCCUUUUUAUAGUCA | 6990 | UGACUAUAAAAAGGGCAAA |
| siRNA 1501 | 1501 | UUGCCCUUUUUAUAGUCAC | 6991 | GUGACUAUAAAAAGGGCAA |
| siRNA 1502 | 1502 | UGCCCUUUUUAUAGUCACC | 6992 | GGUGACUAUAAAAAGGGCA |
| siRNA 1503 | 1503 | GCCCUUUUUAUAGUCACCA | 6993 | UGGUGACUAUAAAAAGGGC |
| siRNA 1504 | 1504 | CCCUUUUUAUAGUCACCAA | 6994 | UUGGUGACUAUAAAAAGGG |
| siRNA 1505 | 1505 | CCUUUUUAUAGUCACCAAU | 6995 | AUUGGUGACUAUAAAAAGG |
| siRNA 1506 | 1506 | CUUUUUAUAGUCACCAAUU | 6996 | AAUUGGUGACUAUAAAAAG |
| siRNA 1507 | 1507 | UUUUUAUAGUCACCAAUUA | 6997 | UAAUUGGUGACUAUAAAAA |
| siRNA 1508 | 1508 | UUUUAUAGUCACCAAUUAA | 6998 | UUAAUUGGUGACUAUAAAA |
| siRNA 1509 | 1509 | UUUAUAGUCACCAAUUAAG | 6999 | CUUAAUUGGUGACUAUAAA |
| siRNA 1510 | 1510 | UUAUAGUCACCAAUUAAGA | 7000 | UCUUAAUUGGUGACUAUAA |
| siRNA 1511 | 1511 | UAUAGUCACCAAUUAAGAA | 7001 | UUCUUAAUUGGUGACUAUA |
| siRNA 1512 | 1512 | AUAGUCACCAAUUAAGAAG | 7002 | CUUCUUAAUUGGUGACUAU |
| siRNA 1513 | 1513 | UAGUCACCAAUUAAGAAGC | 7003 | GCUUCUUAAUUGGUGACUA |
| siRNA 1514 | 1514 | AGUCACCAAUUAAGAAGCA | 7004 | UGCUUCUUAAUUGGUGACU |
| siRNA 1515 | 1515 | GUCACCAAUUAAGAAGCAA | 7005 | UUGCUUCUUAAUUGGUGAC |
| siRNA 1516 | 1516 | UCACCAAUUAAGAAGCAAA | 7006 | UUUGCUUCUUAAUUGGUGA |
| siRNA 1517 | 1517 | CACCAAUUAAGAAGCAAAC | 7007 | GUUUGCUUCUUAAUUGGUG |
| siRNA 1518 | 1518 | ACCAAUUAAGAAGCAAACA | 7008 | UGUUUGCUUCUUAAUUGGU |
| siRNA 1519 | 1519 | CCAAUUAAGAAGCAAACAC | 7009 | GUGUUUGCUUCUUAAUUGG |
| siRNA 1520 | 1520 | CAAUUAAGAAGCAAACACC | 7010 | GGUGUUUGCUUCUUAAUUG |
| siRNA 1521 | 1521 | AAUUAAGAAGCAAACACCU | 7011 | AGGUGUUUGCUUCUUAAUU |
| siRNA 1522 | 1522 | AUUAAGAAGCAAACACCUG | 7012 | CAGGUGUUUGCUUCUUAAU |
| siRNA 1523 | 1523 | UUAAGAAGCAAACACCUGG | 7013 | CCAGGUGUUUGCUUCUUAA |
| siRNA 1524 | 1524 | UAAGAAGCAAACACCUGGC | 7014 | GCCAGGUGUUUGCUUCUUA |
| siRNA 1525 | 1525 | AAGAAGCAAACACCUGGCU | 7015 | AGCCAGGUGUUUGCUUCUU |
| siRNA 1526 | 1526 | AGAAGCAAACACCUGGCUA | 7016 | UAGCCAGGUGUUUGCUUCU |
| siRNA 1527 | 1527 | GAAGCAAACACCUGGCUAU | 7017 | AUAGCCAGGUGUUUGCUUC |
| siRNA 1528 | 1528 | AAGCAAACACCUGGCUAUC | 7018 | GAUAGCCAGGUGUUUGCUU |
| siRNA 1529 | 1529 | AGCAAACACCUGGCUAUCA | 7019 | UGAUAGCCAGGUGUUUGCU |
| siRNA 1530 | 1530 | GCAAACACCUGGCUAUCAA | 7020 | UUGAUAGCCAGGUGUUUGC |
| siRNA 1531 | 1531 | CAAACACCUGGCUAUCAAA | 7021 | UUUGAUAGCCAGGUGUUUG |
| siRNA 1532 | 1532 | AAACACCUGGCUAUCAAAA | 7022 | UUUUGAUAGCCAGGUGUUU |
| siRNA 1533 | 1533 | AACACCUGGCUAUCAAAAA | 7023 | UUUUUGAUAGCCAGGUGUU |
| siRNA 1534 | 1534 | ACACCUGGCUAUCAAAAAA | 7024 | UUUUUUGAUAGCCAGGUGU |
| siRNA 1535 | 1535 | CACCUGGCUAUCAAAAAAA | 7025 | UUUUUUUGAUAGCCAGGUG |
| siRNA 1536 | 1536 | ACCUGGCUAUCAAAAAAAU | 7026 | AUUUUUUUGAUAGCCAGGU |
| siRNA 1537 | 1537 | CCUGGCUAUCAAAAAAAUG | 7027 | CAUUUUUUUGAUAGCCAGG |
| siRNA 1538 | 1538 | CUGGCUAUCAAAAAAAUGA | 7028 | UCAUUUUUUUGAUAGCCAG |
| siRNA 1539 | 1539 | UGGCUAUCAAAAAAAUGAA | 7029 | UUCAUUUUUUUGAUAGCCA |
| siRNA 1540 | 1540 | GGCUAUCAAAAAAAUGAAU | 7030 | AUUCAUUUUUUUGAUAGCC |
| siRNA 1541 | 1541 | GCUAUCAAAAAAAUGAAUG | 7031 | CAUUCAUUUUUUUGAUAGC |
| siRNA 1542 | 1542 | CUAUCAAAAAAAUGAAUGA | 7032 | UCAUUCAUUUUUUUGAUAG |
| siRNA 1543 | 1543 | UAUCAAAAAAAUGAAUGAA | 7033 | UUCAUUCAUUUUUUUGAUA |
| siRNA 1544 | 1544 | AUCAAAAAAAUGAAUGAAA | 7034 | UUUCAUUCAUUUUUUUGAU |
| siRNA 1545 | 1545 | UCAAAAAAAUGAAUGAAAU | 7035 | AUUUCAUUCAUUUUUUUGA |
| siRNA 1546 | 1546 | CAAAAAAAUGAAUGAAAUU | 7036 | AAUUUCAUUCAUUUUUUUG |
| siRNA 1547 | 1547 | AAAAAAAUGAAUGAAAUUC | 7037 | GAAUUUCAUUCAUUUUUUU |
| siRNA 1548 | 1548 | AAAAAAUGAAUGAAAUUCA | 7038 | UGAAUUUCAUUCAUUUUUU |
| siRNA 1549 | 1549 | AAAAAUGAAUGAAAUUCAG | 7039 | CUGAAUUUCAUUCAUUUUU |
| siRNA 1550 | 1550 | AAAAUGAAUGAAAUUCAGA | 7040 | UCUGAAUUUCAUUCAUUUU |
| siRNA 1551 | 1551 | AAAUGAAUGAAAUUCAGAA | 7041 | UUCUGAAUUUCAUUCAUUU |
| siRNA 1552 | 1552 | AAUGAAUGAAAUUCAGAAA | 7042 | UUUCUGAAUUUCAUUCAUU |
| siRNA 1553 | 1553 | AUGAAUGAAAUUCAGAAAA | 7043 | UUUUCUGAAUUUCAUUCAU |
| siRNA 1554 | 1554 | UGAAUGAAAUUCAGAAAAA | 7044 | UUUUUCUGAAUUUCAUUCA |
| siRNA 1555 | 1555 | GAAUGAAAUUCAGAAAAAU | 7045 | AUUUUUCUGAAUUUCAUUC |
| siRNA 1556 | 1556 | AAUGAAAUUCAGAAAAAUA | 7046 | UAUUUUUCUGAAUUUCAUU |
| siRNA 1557 | 1557 | AUGAAAUUCAGAAAAAUAU | 7047 | AUAUUUUUCUGAAUUUCAU |
| siRNA 1558 | 1558 | UGAAAUUCAGAAAAAUAUC | 7048 | GAUAUUUUUCUGAAUUUCA |
| siRNA 1559 | 1559 | GAAAUUCAGAAAAAUAUCG | 7049 | CGAUAUUUUUCUGAAUUUC |
| siRNA 1560 | 1560 | AAAUUCAGAAAAAUAUCGA | 7050 | UCGAUAUUUUUCUGAAUUU |
| siRNA 1561 | 1561 | AAUUCAGAAAAAUAUCGAU | 7051 | AUCGAUAUUUUUCUGAAUU |
| siRNA 1562 | 1562 | AUUCAGAAAAAUAUCGAUG | 7052 | CAUCGAUAUUUUUCUGAAU |
| siRNA 1563 | 1563 | UUCAGAAAAAUAUCGAUGG | 7053 | CCAUCGAUAUUUUUCUGAA |
| siRNA 1564 | 1564 | UCAGAAAAAUAUCGAUGGA | 7054 | UCCAUCGAUAUUUUUCUGA |
| siRNA 1565 | 1565 | CAGAAAAAUAUCGAUGGAU | 7055 | AUCCAUCGAUAUUUUUCUG |
| siRNA 1566 | 1566 | AGAAAAAUAUCGAUGGAUG | 7056 | CAUCCAUCGAUAUUUUUCU |
| siRNA 1567 | 1567 | GAAAAAUAUCGAUGGAUGG | 7057 | CCAUCCAUCGAUAUUUUUC |
| siRNA 1568 | 1568 | AAAAAUAUCGAUGGAUGGG | 7058 | CCCAUCCAUCGAUAUUUUU |
| siRNA 1569 | 1569 | AAAAUAUCGAUGGAUGGGA | 7059 | UCCCAUCCAUCGAUAUUUU |
| siRNA 1570 | 1570 | AAAUAUCGAUGGAUGGGAA | 7060 | UUCCCAUCCAUCGAUAUUU |
| siRNA 1571 | 1571 | AAUAUCGAUGGAUGGGAAG | 7061 | CUUCCCAUCCAUCGAUAUU |
| siRNA 1572 | 1572 | AUAUCGAUGGAUGGGAAGG | 7062 | CCUUCCCAUCCAUCGAUAU |
| siRNA 1573 | 1573 | UAUCGAUGGAUGGGAAGGC | 7063 | GCCUUCCCAUCCAUCGAUA |
| siRNA 1574 | 1574 | AUCGAUGGAUGGGAAGGCA | 7064 | UGCCUUCCCAUCCAUCGAU |
| siRNA 1575 | 1575 | UCGAUGGAUGGGAAGGCAA | 7065 | UUGCCUUCCCAUCCAUCGA |
| siRNA 1576 | 1576 | CGAUGGAUGGGAAGGCAAA | 7066 | UUUGCCUUCCCAUCCAUCG |
| siRNA 1577 | 1577 | GAUGGAUGGGAAGGCAAAG | 7067 | CUUUGCCUUCCCAUCCAUC |
| siRNA 1578 | 1578 | AUGGAUGGGAAGGCAAAGA | 7068 | UCUUUGCCUUCCCAUCCAU |
| siRNA 1579 | 1579 | UGGAUGGGAAGGCAAAGAU | 7069 | AUCUUUGCCUUCCCAUCCA |
| siRNA 1580 | 1580 | GGAUGGGAAGGCAAAGAUA | 7070 | UAUCUUUGCCUUCCCAUCC |
| siRNA 1581 | 1581 | GAUGGGAAGGCAAAGAUAU | 7071 | AUAUCUUUGCCUUCCCAUC |
| siRNA 1582 | 1582 | AUGGGAAGGCAAAGAUAUU | 7072 | AAUAUCUUUGCCUUCCCAU |
| siRNA 1583 | 1583 | UGGGAAGGCAAAGAUAUUG | 7073 | CAAUAUCUUUGCCUUCCCA |
| siRNA 1584 | 1584 | GGGAAGGCAAAGAUAUUGG | 7074 | CCAAUAUCUUUGCCUUCCC |
| siRNA 1585 | 1585 | GGAAGGCAAAGAUAUUGGA | 7075 | UCCAAUAUCUUUGCCUUCC |
| siRNA 1586 | 1586 | GAAGGCAAAGAUAUUGGAC | 7076 | GUCCAAUAUCUUUGCCUUC |
| siRNA 1587 | 1587 | AAGGCAAAGAUAUUGGACA | 7077 | UGUCCAAUAUCUUUGCCUU |
| siRNA 1588 | 1588 | AGGCAAAGAUAUUGGACAG | 7078 | CUGUCCAAUAUCUUUGCCU |
| siRNA 1589 | 1589 | GGCAAAGAUAUUGGACAGU | 7079 | ACUGUCCAAUAUCUUUGCC |
| siRNA 1590 | 1590 | GCAAAGAUAUUGGACAGUG | 7080 | CACUGUCCAAUAUCUUUGC |
| siRNA 1591 | 1591 | CAAAGAUAUUGGACAGUGU | 7081 | ACACUGUCCAAUAUCUUUG |
| siRNA 1592 | 1592 | AAAGAUAUUGGACAGUGUU | 7082 | AACACUGUCCAAUAUCUUU |
| siRNA 1593 | 1593 | AAGAUAUUGGACAGUGUUG | 7083 | CAACACUGUCCAAUAUCUU |
| siRNA 1594 | 1594 | AGAUAUUGGACAGUGUUGU | 7084 | ACAACACUGUCCAAUAUCU |
| siRNA 1595 | 1595 | GAUAUUGGACAGUGUUGUA | 7085 | UACAACACUGUCCAAUAUC |
| siRNA 1596 | 1596 | AUAUUGGACAGUGUUGUAA | 7086 | UUACAACACUGUCCAAUAU |
| siRNA 1597 | 1597 | UAUUGGACAGUGUUGUAAU | 7087 | AUUACAACACUGUCCAAUA |
| siRNA 1598 | 1598 | AUUGGACAGUGUUGUAAUG | 7088 | CAUUACAACACUGUCCAAU |
| siRNA 1599 | 1599 | UUGGACAGUGUUGUAAUGA | 7089 | UCAUUACAACACUGUCCAA |
| siRNA 1600 | 1600 | UGGACAGUGUUGUAAUGAA | 7090 | UUCAUUACAACACUGUCCA |
| siRNA 1601 | 1601 | GGACAGUGUUGUAAUGAAU | 7091 | AUUCAUUACAACACUGUCC |
| siRNA 1602 | 1602 | GACAGUGUUGUAAUGAAUU | 7092 | AAUUCAUUACAACACUGUC |
| siRNA 1603 | 1603 | ACAGUGUUGUAAUGAAUUC | 7093 | GAAUUCAUUACAACACUGU |
| siRNA 1604 | 1604 | CAGUGUUGUAAUGAAUUCA | 7094 | UGAAUUCAUUACAACACUG |
| siRNA 1605 | 1605 | AGUGUUGUAAUGAAUUCAU | 7095 | AUGAAUUCAUUACAACACU |
| siRNA 1606 | 1606 | GUGUUGUAAUGAAUUCAUU | 7096 | AAUGAAUUCAUUACAACAC |
| siRNA 1607 | 1607 | UGUUGUAAUGAAUUCAUUA | 7097 | UAAUGAAUUCAUUACAACA |
| siRNA 1608 | 1608 | GUUGUAAUGAAUUCAUUAU | 7098 | AUAAUGAAUUCAUUACAAC |
| siRNA 1609 | 1609 | UUGUAAUGAAUUCAUUAUG | 7099 | CAUAAUGAAUUCAUUACAA |
| siRNA 1610 | 1610 | UGUAAUGAAUUCAUUAUGG | 7100 | CCAUAAUGAAUUCAUUACA |
| siRNA 1611 | 1611 | GUAAUGAAUUCAUUAUGGA | 7101 | UCCAUAAUGAAUUCAUUAC |
| siRNA 1612 | 1612 | UAAUGAAUUCAUUAUGGAG | 7102 | CUCCAUAAUGAAUUCAUUA |
| siRNA 1613 | 1613 | AAUGAAUUCAUUAUGGAGG | 7103 | CCUCCAUAAUGAAUUCAUU |
| siRNA 1614 | 1614 | AUGAAUUCAUUAUGGAGGG | 7104 | CCCUCCAUAAUGAAUUCAU |
| siRNA 1615 | 1615 | UGAAUUCAUUAUGGAGGGA | 7105 | UCCCUCCAUAAUGAAUUCA |
| siRNA 1616 | 1616 | GAAUUCAUUAUGGAGGGAC | 7106 | GUCCCUCCAUAAUGAAUUC |
| siRNA 1617 | 1617 | AAUUCAUUAUGGAGGGACC | 7107 | GGUCCCUCCAUAAUGAAUU |
| siRNA 1618 | 1618 | AUUCAUUAUGGAGGGACCA | 7108 | UGGUCCCUCCAUAAUGAAU |
| siRNA 1619 | 1619 | UUCAUUAUGGAGGGACCAU | 7109 | AUGGUCCCUCCAUAAUGAA |
| siRNA 1620 | 1620 | UCAUUAUGGAGGGACCAUU | 7110 | AAUGGUCCCUCCAUAAUGA |
| siRNA 1621 | 1621 | CAUUAUGGAGGGACCAUUG | 7111 | CAAUGGUCCCUCCAUAAUG |
| siRNA 1622 | 1622 | AUUAUGGAGGGACCAUUGA | 7112 | UCAAUGGUCCCUCCAUAAU |
| siRNA 1623 | 1623 | UUAUGGAGGGACCAUUGAC | 7113 | GUCAAUGGUCCCUCCAUAA |
| siRNA 1624 | 1624 | UAUGGAGGGACCAUUGACA | 7114 | UGUCAAUGGUCCCUCCAUA |
| siRNA 1625 | 1625 | AUGGAGGGACCAUUGACAA | 7115 | UUGUCAAUGGUCCCUCCAU |
| siRNA 1626 | 1626 | UGGAGGGACCAUUGACAAG | 7116 | CUUGUCAAUGGUCCCUCCA |
| siRNA 1627 | 1627 | GGAGGGACCAUUGACAAGA | 7117 | UCUUGUCAAUGGUCCCUCC |
| siRNA 1628 | 1628 | GAGGGACCAUUGACAAGAA | 7118 | UUCUUGUCAAUGGUCCCUC |
| siRNA 1629 | 1629 | AGGGACCAUUGACAAGAAU | 7119 | AUUCUUGUCAAUGGUCCCU |
| siRNA 1630 | 1630 | GGGACCAUUGACAAGAAUC | 7120 | GAUUCUUGUCAAUGGUCCC |
| siRNA 1631 | 1631 | GGACCAUUGACAAGAAUCG | 7121 | CGAUUCUUGUCAAUGGUCC |
| siRNA 1632 | 1632 | GACCAUUGACAAGAAUCGG | 7122 | CCGAUUCUUGUCAAUGGUC |
| siRNA 1633 | 1633 | ACCAUUGACAAGAAUCGGU | 7123 | ACCGAUUCUUGUCAAUGGU |
| siRNA 1634 | 1634 | CCAUUGACAAGAAUCGGUG | 7124 | CACCGAUUCUUGUCAAUGG |
| siRNA 1635 | 1635 | CAUUGACAAGAAUCGGUGC | 7125 | GCACCGAUUCUUGUCAAUG |
| siRNA 1636 | 1636 | AUUGACAAGAAUCGGUGCC | 7126 | GGCACCGAUUCUUGUCAAU |
| siRNA 1637 | 1637 | UUGACAAGAAUCGGUGCCA | 7127 | UGGCACCGAUUCUUGUCAA |
| siRNA 1638 | 1638 | UGACAAGAAUCGGUGCCAA | 7128 | UUGGCACCGAUUCUUGUCA |
| siRNA 1639 | 1639 | GACAAGAAUCGGUGCCAAA | 7129 | UUUGGCACCGAUUCUUGUC |
| siRNA 1640 | 1640 | ACAAGAAUCGGUGCCAAAC | 7130 | GUUUGGCACCGAUUCUUGU |
| siRNA 1641 | 1641 | CAAGAAUCGGUGCCAAACA | 7131 | UGUUUGGCACCGAUUCUUG |
| siRNA 1642 | 1642 | AAGAAUCGGUGCCAAACAU | 7132 | AUGUUUGGCACCGAUUCUU |
| siRNA 1643 | 1643 | AGAAUCGGUGCCAAACAUG | 7133 | CAUGUUUGGCACCGAUUCU |
| siRNA 1644 | 1644 | GAAUCGGUGCCAAACAUGA | 7134 | UCAUGUUUGGCACCGAUUC |
| siRNA 1645 | 1645 | AAUCGGUGCCAAACAUGAA | 7135 | UUCAUGUUUGGCACCGAUU |
| siRNA 1646 | 1646 | AUCGGUGCCAAACAUGAAC | 7136 | GUUCAUGUUUGGCACCGAU |
| siRNA 1647 | 1647 | UCGGUGCCAAACAUGAACG | 7137 | CGUUCAUGUUUGGCACCGA |
| siRNA 1648 | 1648 | CGGUGCCAAACAUGAACGG | 7138 | CCGUUCAUGUUUGGCACCG |
| siRNA 1649 | 1649 | GGUGCCAAACAUGAACGGC | 7139 | GCCGUUCAUGUUUGGCACC |
| siRNA 1650 | 1650 | GUGCCAAACAUGAACGGCA | 7140 | UGCCGUUCAUGUUUGGCAC |
| siRNA 1651 | 1651 | UGCCAAACAUGAACGGCAU | 7141 | AUGCCGUUCAUGUUUGGCA |
| siRNA 1652 | 1652 | GCCAAACAUGAACGGCAUA | 7142 | UAUGCCGUUCAUGUUUGGC |
| siRNA 1653 | 1653 | CCAAACAUGAACGGCAUAU | 7143 | AUAUGCCGUUCAUGUUUGG |
| siRNA 1654 | 1654 | CAAACAUGAACGGCAUAUU | 7144 | AAUAUGCCGUUCAUGUUUG |
| siRNA 1655 | 1655 | AAACAUGAACGGCAUAUUU | 7145 | AAAUAUGCCGUUCAUGUUU |
| siRNA 1656 | 1656 | AACAUGAACGGCAUAUUUU | 7146 | AAAAUAUGCCGUUCAUGUU |
| siRNA 1657 | 1657 | ACAUGAACGGCAUAUUUUU | 7147 | AAAAAUAUGCCGUUCAUGU |
| siRNA 1658 | 1658 | CAUGAACGGCAUAUUUUUC | 7148 | GAAAAAUAUGCCGUUCAUG |
| siRNA 1659 | 1659 | AUGAACGGCAUAUUUUUCU | 7149 | AGAAAAAUAUGCCGUUCAU |
| siRNA 1660 | 1660 | UGAACGGCAUAUUUUUCUG | 7150 | CAGAAAAAUAUGCCGUUCA |
| siRNA 1661 | 1661 | GAACGGCAUAUUUUUCUGU | 7151 | ACAGAAAAAUAUGCCGUUC |
| siRNA 1662 | 1662 | AACGGCAUAUUUUUCUGUU | 7152 | AACAGAAAAAUAUGCCGUU |
| siRNA 1663 | 1663 | ACGGCAUAUUUUUCUGUUU | 7153 | AAACAGAAAAAUAUGCCGU |
| siRNA 1664 | 1664 | CGGCAUAUUUUUCUGUUUG | 7154 | CAAACAGAAAAAUAUGCCG |
| siRNA 1665 | 1665 | GGCAUAUUUUUCUGUUUGA | 7155 | UCAAACAGAAAAAUAUGCC |
| siRNA 1666 | 1666 | GCAUAUUUUUCUGUUUGAU | 7156 | AUCAAACAGAAAAAUAUGC |
| siRNA 1667 | 1667 | CAUAUUUUUCUGUUUGAUG | 7157 | CAUCAAACAGAAAAAUAUG |
| siRNA 1668 | 1668 | AUAUUUUUCUGUUUGAUGG | 7158 | CCAUCAAACAGAAAAAUAU |
| siRNA 1669 | 1669 | UAUUUUUCUGUUUGAUGGC | 7159 | GCCAUCAAACAGAAAAAUA |
| siRNA 1670 | 1670 | AUUUUUCUGUUUGAUGGCU | 7160 | AGCCAUCAAACAGAAAAAU |
| siRNA 1671 | 1671 | UUUUUCUGUUUGAUGGCUU | 7161 | AAGCCAUCAAACAGAAAAA |
| siRNA 1672 | 1672 | UUUUCUGUUUGAUGGCUUA | 7162 | UAAGCCAUCAAACAGAAAA |
| siRNA 1673 | 1673 | UUUCUGUUUGAUGGCUUAA | 7163 | UUAAGCCAUCAAACAGAAA |
| siRNA 1674 | 1674 | UUCUGUUUGAUGGCUUAAU | 7164 | AUUAAGCCAUCAAACAGAA |
| siRNA 1675 | 1675 | UCUGUUUGAUGGCUUAAUG | 7165 | CAUUAAGCCAUCAAACAGA |
| siRNA 1676 | 1676 | CUGUUUGAUGGCUUAAUGA | 7166 | UCAUUAAGCCAUCAAACAG |
| siRNA 1677 | 1677 | UGUUUGAUGGCUUAAUGAU | 7167 | AUCAUUAAGCCAUCAAACA |
| siRNA 1678 | 1678 | GUUUGAUGGCUUAAUGAUC | 7168 | GAUCAUUAAGCCAUCAAAC |
| siRNA 1679 | 1679 | UUUGAUGGCUUAAUGAUCA | 7169 | UGAUCAUUAAGCCAUCAAA |
| siRNA 1680 | 1680 | UUGAUGGCUUAAUGAUCAG | 7170 | CUGAUCAUUAAGCCAUCAA |
| siRNA 1681 | 1681 | UGAUGGCUUAAUGAUCAGU | 7171 | ACUGAUCAUUAAGCCAUCA |
| siRNA 1682 | 1682 | GAUGGCUUAAUGAUCAGUU | 7172 | AACUGAUCAUUAAGCCAUC |
| siRNA 1683 | 1683 | AUGGCUUAAUGAUCAGUUG | 7173 | CAACUGAUCAUUAAGCCAU |
| siRNA 1684 | 1684 | UGGCUUAAUGAUCAGUUGU | 7174 | ACAACUGAUCAUUAAGCCA |
| siRNA 1685 | 1685 | GGCUUAAUGAUCAGUUGUA | 7175 | UACAACUGAUCAUUAAGCC |
| siRNA 1686 | 1686 | GCUUAAUGAUCAGUUGUAA | 7176 | UUACAACUGAUCAUUAAGC |
| siRNA 1687 | 1687 | CUUAAUGAUCAGUUGUAAA | 7177 | UUUACAACUGAUCAUUAAG |
| siRNA 1688 | 1688 | UUAAUGAUCAGUUGUAAAC | 7178 | GUUUACAACUGAUCAUUAA |
| siRNA 1689 | 1689 | UAAUGAUCAGUUGUAAACC | 7179 | GGUUUACAACUGAUCAUUA |
| siRNA 1690 | 1690 | AAUGAUCAGUUGUAAACCU | 7180 | AGGUUUACAACUGAUCAUU |
| siRNA 1691 | 1691 | AUGAUCAGUUGUAAACCUA | 7181 | UAGGUUUACAACUGAUCAU |
| siRNA 1692 | 1692 | UGAUCAGUUGUAAACCUAA | 7182 | UUAGGUUUACAACUGAUCA |
| siRNA 1693 | 1693 | GAUCAGUUGUAAACCUAAU | 7183 | AUUAGGUUUACAACUGAUC |
| siRNA 1694 | 1694 | AUCAGUUGUAAACCUAAUC | 7184 | GAUUAGGUUUACAACUGAU |
| siRNA 1695 | 1695 | UCAGUUGUAAACCUAAUCA | 7185 | UGAUUAGGUUUACAACUGA |
| siRNA 1696 | 1696 | CAGUUGUAAACCUAAUCAU | 7186 | AUGAUUAGGUUUACAACUG |
| siRNA 1697 | 1697 | AGUUGUAAACCUAAUCAUG | 7187 | CAUGAUUAGGUUUACAACU |
| siRNA 1698 | 1698 | GUUGUAAACCUAAUCAUGG | 7188 | CCAUGAUUAGGUUUACAAC |
| siRNA 1699 | 1699 | UUGUAAACCUAAUCAUGGC | 7189 | GCCAUGAUUAGGUUUACAA |
| siRNA 1700 | 1700 | UGUAAACCUAAUCAUGGCC | 7190 | GGCCAUGAUUAGGUUUACA |
| siRNA 1701 | 1701 | GUAAACCUAAUCAUGGCCA | 7191 | UGGCCAUGAUUAGGUUUAC |
| siRNA 1702 | 1702 | UAAACCUAAUCAUGGCCAG | 7192 | CUGGCCAUGAUUAGGUUUA |
| siRNA 1703 | 1703 | AAACCUAAUCAUGGCCAGA | 7193 | UCUGGCCAUGAUUAGGUUU |
| siRNA 1704 | 1704 | AACCUAAUCAUGGCCAGAC | 7194 | GUCUGGCCAUGAUUAGGUU |
| siRNA 1705 | 1705 | ACCUAAUCAUGGCCAGACU | 7195 | AGUCUGGCCAUGAUUAGGU |
| siRNA 1706 | 1706 | CCUAAUCAUGGCCAGACUC | 7196 | GAGUCUGGCCAUGAUUAGG |
| siRNA 1707 | 1707 | CUAAUCAUGGCCAGACUCG | 7197 | CGAGUCUGGCCAUGAUUAG |
| siRNA 1708 | 1708 | UAAUCAUGGCCAGACUCGG | 7198 | CCGAGUCUGGCCAUGAUUA |
| siRNA 1709 | 1709 | AAUCAUGGCCAGACUCGGC | 7199 | GCCGAGUCUGGCCAUGAUU |
| siRNA 1710 | 1710 | AUCAUGGCCAGACUCGGCU | 7200 | AGCCGAGUCUGGCCAUGAU |
| siRNA 1711 | 1711 | UCAUGGCCAGACUCGGCUU | 7201 | AAGCCGAGUCUGGCCAUGA |
| siRNA 1712 | 1712 | CAUGGCCAGACUCGGCUUC | 7202 | GAAGCCGAGUCUGGCCAUG |
| siRNA 1713 | 1713 | AUGGCCAGACUCGGCUUCC | 7203 | GGAAGCCGAGUCUGGCCAU |
| siRNA 1714 | 1714 | UGGCCAGACUCGGCUUCCA | 7204 | UGGAAGCCGAGUCUGGCCA |
| siRNA 1715 | 1715 | GGCCAGACUCGGCUUCCAG | 7205 | CUGGAAGCCGAGUCUGGCC |
| siRNA 1716 | 1716 | GCCAGACUCGGCUUCCAGG | 7206 | CCUGGAAGCCGAGUCUGGC |
| siRNA 1717 | 1717 | CCAGACUCGGCUUCCAGGU | 7207 | ACCUGGAAGCCGAGUCUGG |
| siRNA 1718 | 1718 | CAGACUCGGCUUCCAGGUU | 7208 | AACCUGGAAGCCGAGUCUG |
| siRNA 1719 | 1719 | AGACUCGGCUUCCAGGUUA | 7209 | UAACCUGGAAGCCGAGUCU |
| siRNA 1720 | 1720 | GACUCGGCUUCCAGGUUAC | 7210 | GUAACCUGGAAGCCGAGUC |
| siRNA 1721 | 1721 | ACUCGGCUUCCAGGUUACA | 7211 | UGUAACCUGGAAGCCGAGU |
| siRNA 1722 | 1722 | CUCGGCUUCCAGGUUACAG | 7212 | CUGUAACCUGGAAGCCGAG |
| siRNA 1723 | 1723 | UCGGCUUCCAGGUUACAGU | 7213 | ACUGUAACCUGGAAGCCGA |
| siRNA 1724 | 1724 | CGGCUUCCAGGUUACAGUA | 7214 | UACUGUAACCUGGAAGCCG |
| siRNA 1725 | 1725 | GGCUUCCAGGUUACAGUAG | 7215 | CUACUGUAACCUGGAAGCC |
| siRNA 1726 | 1726 | GCUUCCAGGUUACAGUAGU | 7216 | ACUACUGUAACCUGGAAGC |
| siRNA 1727 | 1727 | CUUCCAGGUUACAGUAGUG | 7217 | CACUACUGUAACCUGGAAG |
| siRNA 1728 | 1728 | UUCCAGGUUACAGUAGUGC | 7218 | GCACUACUGUAACCUGGAA |
| siRNA 1729 | 1729 | UCCAGGUUACAGUAGUGCA | 7219 | UGCACUACUGUAACCUGGA |
| siRNA 1730 | 1730 | CCAGGUUACAGUAGUGCAG | 7220 | CUGCACUACUGUAACCUGG |
| siRNA 1731 | 1731 | CAGGUUACAGUAGUGCAGA | 7221 | UCUGCACUACUGUAACCUG |
| siRNA 1732 | 1732 | AGGUUACAGUAGUGCAGAA | 7222 | UUCUGCACUACUGUAACCU |
| siRNA 1733 | 1733 | GGUUACAGUAGUGCAGAAU | 7223 | AUUCUGCACUACUGUAACC |
| siRNA 1734 | 1734 | GUUACAGUAGUGCAGAAUA | 7224 | UAUUCUGCACUACUGUAAC |
| siRNA 1735 | 1735 | UUACAGUAGUGCAGAAUAC | 7225 | GUAUUCUGCACUACUGUAA |
| siRNA 1736 | 1736 | UACAGUAGUGCAGAAUACA | 7226 | UGUAUUCUGCACUACUGUA |
| siRNA 1737 | 1737 | ACAGUAGUGCAGAAUACAG | 7227 | CUGUAUUCUGCACUACUGU |
| siRNA 1738 | 1738 | CAGUAGUGCAGAAUACAGG | 7228 | CCUGUAUUCUGCACUACUG |
| siRNA 1739 | 1739 | AGUAGUGCAGAAUACAGGU | 7229 | ACCUGUAUUCUGCACUACU |
| siRNA 1740 | 1740 | GUAGUGCAGAAUACAGGUU | 7230 | AACCUGUAUUCUGCACUAC |
| siRNA 1741 | 1741 | UAGUGCAGAAUACAGGUUA | 7231 | UAACCUGUAUUCUGCACUA |
| siRNA 1742 | 1742 | AGUGCAGAAUACAGGUUAA | 7232 | UUAACCUGUAUUCUGCACU |
| siRNA 1743 | 1743 | GUGCAGAAUACAGGUUAAA | 7233 | UUUAACCUGUAUUCUGCAC |
| siRNA 1744 | 1744 | UGCAGAAUACAGGUUAAAA | 7234 | UUUUAACCUGUAUUCUGCA |
| siRNA 1745 | 1745 | GCAGAAUACAGGUUAAAAG | 7235 | CUUUUAACCUGUAUUCUGC |
| siRNA 1746 | 1746 | CAGAAUACAGGUUAAAAGA | 7236 | UCUUUUAACCUGUAUUCUG |
| siRNA 1747 | 1747 | AGAAUACAGGUUAAAAGAA | 7237 | UUCUUUUAACCUGUAUUCU |
| siRNA 1748 | 1748 | GAAUACAGGUUAAAAGAAA | 7238 | UUUCUUUUAACCUGUAUUC |
| siRNA 1749 | 1749 | AAUACAGGUUAAAAGAAAA | 7239 | UUUUCUUUUAACCUGUAUU |
| siRNA 1750 | 1750 | AUACAGGUUAAAAGAAAAA | 7240 | UUUUUCUUUUAACCUGUAU |
| siRNA 1751 | 1751 | UACAGGUUAAAAGAAAAAU | 7241 | AUUUUUCUUUUAACCUGUA |
| siRNA 1752 | 1752 | ACAGGUUAAAAGAAAAAUU | 7242 | AAUUUUUCUUUUAACCUGU |
| siRNA 1753 | 1753 | CAGGUUAAAAGAAAAAUUU | 7243 | AAAUUUUUCUUUUAACCUG |
| siRNA 1754 | 1754 | AGGUUAAAAGAAAAAUUUG | 7244 | CAAAUUUUUCUUUUAACCU |
| siRNA 1755 | 1755 | GGUUAAAAGAAAAAUUUGU | 7245 | ACAAAUUUUUCUUUUAACC |
| siRNA 1756 | 1756 | GUUAAAAGAAAAAUUUGUC | 7246 | GACAAAUUUUUCUUUUAAC |
| siRNA 1757 | 1757 | UUAAAAGAAAAAUUUGUCA | 7247 | UGACAAAUUUUUCUUUUAA |
| siRNA 1758 | 1758 | UAAAAGAAAAAUUUGUCAU | 7248 | AUGACAAAUUUUUCUUUUA |
| siRNA 1759 | 1759 | AAAAGAAAAAUUUGUCAUG | 7249 | CAUGACAAAUUUUUCUUUU |
| siRNA 1760 | 1760 | AAAGAAAAAUUUGUCAUGA | 7250 | UCAUGACAAAUUUUUCUUU |
| siRNA 1761 | 1761 | AAGAAAAAUUUGUCAUGAG | 7251 | CUCAUGACAAAUUUUUCUU |
| siRNA 1762 | 1762 | AGAAAAAUUUGUCAUGAGG | 7252 | CCUCAUGACAAAUUUUUCU |
| siRNA 1763 | 1763 | GAAAAAUUUGUCAUGAGGA | 7253 | UCCUCAUGACAAAUUUUUC |
| siRNA 1764 | 1764 | AAAAAUUUGUCAUGAGGAA | 7254 | UUCCUCAUGACAAAUUUUU |
| siRNA 1765 | 1765 | AAAAUUUGUCAUGAGGAAA | 7255 | UUUCCUCAUGACAAAUUUU |
| siRNA 1766 | 1766 | AAAUUUGUCAUGAGGAAAA | 7256 | UUUUCCUCAUGACAAAUUU |
| siRNA 1767 | 1767 | AAUUUGUCAUGAGGAAAAU | 7257 | AUUUUCCUCAUGACAAAUU |
| siRNA 1768 | 1768 | AUUUGUCAUGAGGAAAAUA | 7258 | UAUUUUCCUCAUGACAAAU |
| siRNA 1769 | 1769 | UUUGUCAUGAGGAAAAUAC | 7259 | GUAUUUUCCUCAUGACAAA |
| siRNA 1770 | 1770 | UUGUCAUGAGGAAAAUACA | 7260 | UGUAUUUUCCUCAUGACAA |
| siRNA 1771 | 1771 | UGUCAUGAGGAAAAUACAA | 7261 | UUGUAUUUUCCUCAUGACA |
| siRNA 1772 | 1772 | GUCAUGAGGAAAAUACAAA | 7262 | UUUGUAUUUUCCUCAUGAC |
| siRNA 1773 | 1773 | UCAUGAGGAAAAUACAAAU | 7263 | AUUUGUAUUUUCCUCAUGA |
| siRNA 1774 | 1774 | CAUGAGGAAAAUACAAAUU | 7264 | AAUUUGUAUUUUCCUCAUG |
| siRNA 1775 | 1775 | AUGAGGAAAAUACAAAUUU | 7265 | AAAUUUGUAUUUUCCUCAU |
| siRNA 1776 | 1776 | UGAGGAAAAUACAAAUUUG | 7266 | CAAAUUUGUAUUUUCCUCA |
| siRNA 1777 | 1777 | GAGGAAAAUACAAAUUUGU | 7267 | ACAAAUUUGUAUUUUCCUC |
| siRNA 1778 | 1778 | AGGAAAAUACAAAUUUGUG | 7268 | CACAAAUUUGUAUUUUCCU |
| siRNA 1779 | 1779 | GGAAAAUACAAAUUUGUGA | 7269 | UCACAAAUUUGUAUUUUCC |
| siRNA 1780 | 1780 | GAAAAUACAAAUUUGUGAU | 7270 | AUCACAAAUUUGUAUUUUC |
| siRNA 1781 | 1781 | AAAAUACAAAUUUGUGAUA | 7271 | UAUCACAAAUUUGUAUUUU |
| siRNA 1782 | 1782 | AAAUACAAAUUUGUGAUAA | 7272 | UUAUCACAAAUUUGUAUUU |
| siRNA 1783 | 1783 | AAUACAAAUUUGUGAUAAA | 7273 | UUUAUCACAAAUUUGUAUU |
| siRNA 1784 | 1784 | AUACAAAUUUGUGAUAAAG | 7274 | CUUUAUCACAAAUUUGUAU |
| siRNA 1785 | 1785 | UACAAAUUUGUGAUAAAGA | 7275 | UCUUUAUCACAAAUUUGUA |
| siRNA 1786 | 1786 | ACAAAUUUGUGAUAAAGAA | 7276 | UUCUUUAUCACAAAUUUGU |
| siRNA 1787 | 1787 | CAAAUUUGUGAUAAAGAAG | 7277 | CUUCUUUAUCACAAAUUUG |
| siRNA 1788 | 1788 | AAAUUUGUGAUAAAGAAGA | 7278 | UCUUCUUUAUCACAAAUUU |
| siRNA 1789 | 1789 | AAUUUGUGAUAAAGAAGAU | 7279 | AUCUUCUUUAUCACAAAUU |
| siRNA 1790 | 1790 | AUUUGUGAUAAAGAAGAUA | 7280 | UAUCUUCUUUAUCACAAAU |
| siRNA 1791 | 1791 | UUUGUGAUAAAGAAGAUAC | 7281 | GUAUCUUCUUUAUCACAAA |
| siRNA 1792 | 1792 | UUGUGAUAAAGAAGAUACU | 7282 | AGUAUCUUCUUUAUCACAA |
| siRNA 1793 | 1793 | UGUGAUAAAGAAGAUACUU | 7283 | AAGUAUCUUCUUUAUCACA |
| siRNA 1794 | 1794 | GUGAUAAAGAAGAUACUUG | 7284 | CAAGUAUCUUCUUUAUCAC |
| siRNA 1795 | 1795 | UGAUAAAGAAGAUACUUGU | 7285 | ACAAGUAUCUUCUUUAUCA |
| siRNA 1796 | 1796 | GAUAAAGAAGAUACUUGUG | 7286 | CACAAGUAUCUUCUUUAUC |
| siRNA 1797 | 1797 | AUAAAGAAGAUACUUGUGA | 7287 | UCACAAGUAUCUUCUUUAU |
| siRNA 1798 | 1798 | UAAAGAAGAUACUUGUGAG | 7288 | CUCACAAGUAUCUUCUUUA |
| siRNA 1799 | 1799 | AAAGAAGAUACUUGUGAGC | 7289 | GCUCACAAGUAUCUUCUUU |
| siRNA 1800 | 1800 | AAGAAGAUACUUGUGAGCA | 7290 | UGCUCACAAGUAUCUUCUU |
| siRNA 1801 | 1801 | AGAAGAUACUUGUGAGCAC | 7291 | GUGCUCACAAGUAUCUUCU |
| siRNA 1802 | 1802 | GAAGAUACUUGUGAGCACA | 7292 | UGUGCUCACAAGUAUCUUC |
| siRNA 1803 | 1803 | AAGAUACUUGUGAGCACAA | 7293 | UUGUGCUCACAAGUAUCUU |
| siRNA 1804 | 1804 | AGAUACUUGUGAGCACAAG | 7294 | CUUGUGCUCACAAGUAUCU |
| siRNA 1805 | 1805 | GAUACUUGUGAGCACAAGC | 7295 | GCUUGUGCUCACAAGUAUC |
| siRNA 1806 | 1806 | AUACUUGUGAGCACAAGCA | 7296 | UGCUUGUGCUCACAAGUAU |
| siRNA 1807 | 1807 | UACUUGUGAGCACAAGCAU | 7297 | AUGCUUGUGCUCACAAGUA |
| siRNA 1808 | 1808 | ACUUGUGAGCACAAGCAUG | 7298 | CAUGCUUGUGCUCACAAGU |
| siRNA 1809 | 1809 | CUUGUGAGCACAAGCAUGC | 7299 | GCAUGCUUGUGCUCACAAG |
| siRNA 1810 | 1810 | UUGUGAGCACAAGCAUGCA | 7300 | UGCAUGCUUGUGCUCACAA |
| siRNA 1811 | 1811 | UGUGAGCACAAGCAUGCAU | 7301 | AUGCAUGCUUGUGCUCACA |
| siRNA 1812 | 1812 | GUGAGCACAAGCAUGCAUU | 7302 | AAUGCAUGCUUGUGCUCAC |
| siRNA 1813 | 1813 | UGAGCACAAGCAUGCAUUU | 7303 | AAAUGCAUGCUUGUGCUCA |
| siRNA 1814 | 1814 | GAGCACAAGCAUGCAUUUG | 7304 | CAAAUGCAUGCUUGUGCUC |
| siRNA 1815 | 1815 | AGCACAAGCAUGCAUUUGA | 7305 | UCAAAUGCAUGCUUGUGCU |
| siRNA 1816 | 1816 | GCACAAGCAUGCAUUUGAA | 7306 | UUCAAAUGCAUGCUUGUGC |
| siRNA 1817 | 1817 | CACAAGCAUGCAUUUGAAU | 7307 | AUUCAAAUGCAUGCUUGUG |
| siRNA 1818 | 1818 | ACAAGCAUGCAUUUGAAUU | 7308 | AAUUCAAAUGCAUGCUUGU |
| siRNA 1819 | 1819 | CAAGCAUGCAUUUGAAUUA | 7309 | UAAUUCAAAUGCAUGCUUG |
| siRNA 1820 | 1820 | AAGCAUGCAUUUGAAUUAG | 7310 | CUAAUUCAAAUGCAUGCUU |
| siRNA 1821 | 1821 | AGCAUGCAUUUGAAUUAGU | 7311 | ACUAAUUCAAAUGCAUGCU |
| siRNA 1822 | 1822 | GCAUGCAUUUGAAUUAGUA | 7312 | UACUAAUUCAAAUGCAUGC |
| siRNA 1823 | 1823 | CAUGCAUUUGAAUUAGUAU | 7313 | AUACUAAUUCAAAUGCAUG |
| siRNA 1824 | 1824 | AUGCAUUUGAAUUAGUAUC | 7314 | GAUACUAAUUCAAAUGCAU |
| siRNA 1825 | 1825 | UGCAUUUGAAUUAGUAUCC | 7315 | GGAUACUAAUUCAAAUGCA |
| siRNA 1826 | 1826 | GCAUUUGAAUUAGUAUCCA | 7316 | UGGAUACUAAUUCAAAUGC |
| siRNA 1827 | 1827 | CAUUUGAAUUAGUAUCCAA | 7317 | UUGGAUACUAAUUCAAAUG |
| siRNA 1828 | 1828 | AUUUGAAUUAGUAUCCAAA | 7318 | UUUGGAUACUAAUUCAAAU |
| siRNA 1829 | 1829 | UUUGAAUUAGUAUCCAAAG | 7319 | CUUUGGAUACUAAUUCAAA |
| siRNA 1830 | 1830 | UUGAAUUAGUAUCCAAAGA | 7320 | UCUUUGGAUACUAAUUCAA |
| siRNA 1831 | 1831 | UGAAUUAGUAUCCAAAGAU | 7321 | AUCUUUGGAUACUAAUUCA |
| siRNA 1832 | 1832 | GAAUUAGUAUCCAAAGAUG | 7322 | CAUCUUUGGAUACUAAUUC |
| siRNA 1833 | 1833 | AAUUAGUAUCCAAAGAUGA | 7323 | UCAUCUUUGGAUACUAAUU |
| siRNA 1834 | 1834 | AUUAGUAUCCAAAGAUGAG | 7324 | CUCAUCUUUGGAUACUAAU |
| siRNA 1835 | 1835 | UUAGUAUCCAAAGAUGAGA | 7325 | UCUCAUCUUUGGAUACUAA |
| siRNA 1836 | 1836 | UAGUAUCCAAAGAUGAGAA | 7326 | UUCUCAUCUUUGGAUACUA |
| siRNA 1837 | 1837 | AGUAUCCAAAGAUGAGAAC | 7327 | GUUCUCAUCUUUGGAUACU |
| siRNA 1838 | 1838 | GUAUCCAAAGAUGAGAACA | 7328 | UGUUCUCAUCUUUGGAUAC |
| siRNA 1839 | 1839 | UAUCCAAAGAUGAGAACAG | 7329 | CUGUUCUCAUCUUUGGAUA |
| siRNA 1840 | 1840 | AUCCAAAGAUGAGAACAGC | 7330 | GCUGUUCUCAUCUUUGGAU |
| siRNA 1841 | 1841 | UCCAAAGAUGAGAACAGCA | 7331 | UGCUGUUCUCAUCUUUGGA |
| siRNA 1842 | 1842 | CCAAAGAUGAGAACAGCAU | 7332 | AUGCUGUUCUCAUCUUUGG |
| siRNA 1843 | 1843 | CAAAGAUGAGAACAGCAUA | 7333 | UAUGCUGUUCUCAUCUUUG |
| siRNA 1844 | 1844 | AAAGAUGAGAACAGCAUAA | 7334 | UUAUGCUGUUCUCAUCUUU |
| siRNA 1845 | 1845 | AAGAUGAGAACAGCAUAAU | 7335 | AUUAUGCUGUUCUCAUCUU |
| siRNA 1846 | 1846 | AGAUGAGAACAGCAUAAUA | 7336 | UAUUAUGCUGUUCUCAUCU |
| siRNA 1847 | 1847 | GAUGAGAACAGCAUAAUAU | 7337 | AUAUUAUGCUGUUCUCAUC |
| siRNA 1848 | 1848 | AUGAGAACAGCAUAAUAUU | 7338 | AAUAUUAUGCUGUUCUCAU |
| siRNA 1849 | 1849 | UGAGAACAGCAUAAUAUUU | 7339 | AAAUAUUAUGCUGUUCUCA |
| siRNA 1850 | 1850 | GAGAACAGCAUAAUAUUUG | 7340 | CAAAUAUUAUGCUGUUCUC |
| siRNA 1851 | 1851 | AGAACAGCAUAAUAUUUGC | 7341 | GCAAAUAUUAUGCUGUUCU |
| siRNA 1852 | 1852 | GAACAGCAUAAUAUUUGCU | 7342 | AGCAAAUAUUAUGCUGUUC |
| siRNA 1853 | 1853 | AACAGCAUAAUAUUUGCUG | 7343 | CAGCAAAUAUUAUGCUGUU |
| siRNA 1854 | 1854 | ACAGCAUAAUAUUUGCUGC | 7344 | GCAGCAAAUAUUAUGCUGU |
| siRNA 1855 | 1855 | CAGCAUAAUAUUUGCUGCU | 7345 | AGCAGCAAAUAUUAUGCUG |
| siRNA 1856 | 1856 | AGCAUAAUAUUUGCUGCUA | 7346 | UAGCAGCAAAUAUUAUGCU |
| siRNA 1857 | 1857 | GCAUAAUAUUUGCUGCUAA | 7347 | UUAGCAGCAAAUAUUAUGC |
| siRNA 1858 | 1858 | CAUAAUAUUUGCUGCUAAG | 7348 | CUUAGCAGCAAAUAUUAUG |
| siRNA 1859 | 1859 | AUAAUAUUUGCUGCUAAGU | 7349 | ACUUAGCAGCAAAUAUUAU |
| siRNA 1860 | 1860 | UAAUAUUUGCUGCUAAGUC | 7350 | GACUUAGCAGCAAAUAUUA |
| siRNA 1861 | 1861 | AAUAUUUGCUGCUAAGUCU | 7351 | AGACUUAGCAGCAAAUAUU |
| siRNA 1862 | 1862 | AUAUUUGCUGCUAAGUCUG | 7352 | CAGACUUAGCAGCAAAUAU |
| siRNA 1863 | 1863 | UAUUUGCUGCUAAGUCUGC | 7353 | GCAGACUUAGCAGCAAAUA |
| siRNA 1864 | 1864 | AUUUGCUGCUAAGUCUGCU | 7354 | AGCAGACUUAGCAGCAAAU |
| siRNA 1865 | 1865 | UUUGCUGCUAAGUCUGCUG | 7355 | CAGCAGACUUAGCAGCAAA |
| siRNA 1866 | 1866 | UUGCUGCUAAGUCUGCUGA | 7356 | UCAGCAGACUUAGCAGCAA |
| siRNA 1867 | 1867 | UGCUGCUAAGUCUGCUGAA | 7357 | UUCAGCAGACUUAGCAGCA |
| siRNA 1868 | 1868 | GCUGCUAAGUCUGCUGAAG | 7358 | CUUCAGCAGACUUAGCAGC |
| siRNA 1869 | 1869 | CUGCUAAGUCUGCUGAAGA | 7359 | UCUUCAGCAGACUUAGCAG |
| siRNA 1870 | 1870 | UGCUAAGUCUGCUGAAGAA | 7360 | UUCUUCAGCAGACUUAGCA |
| siRNA 1871 | 1871 | GCUAAGUCUGCUGAAGAAA | 7361 | UUUCUUCAGCAGACUUAGC |
| siRNA 1872 | 1872 | CUAAGUCUGCUGAAGAAAA | 7362 | UUUUCUUCAGCAGACUUAG |
| siRNA 1873 | 1873 | UAAGUCUGCUGAAGAAAAA | 7363 | UUUUUCUUCAGCAGACUUA |
| siRNA 1874 | 1874 | AAGUCUGCUGAAGAAAAAA | 7364 | UUUUUUCUUCAGCAGACUU |
| siRNA 1875 | 1875 | AGUCUGCUGAAGAAAAAAA | 7365 | UUUUUUUCUUCAGCAGACU |
| siRNA 1876 | 1876 | GUCUGCUGAAGAAAAAAAC | 7366 | GUUUUUUUCUUCAGCAGAC |
| siRNA 1877 | 1877 | UCUGCUGAAGAAAAAAACA | 7367 | UGUUUUUUUCUUCAGCAGA |
| siRNA 1878 | 1878 | CUGCUGAAGAAAAAAACAA | 7368 | UUGUUUUUUUCUUCAGCAG |
| siRNA 1879 | 1879 | UGCUGAAGAAAAAAACAAC | 7369 | GUUGUUUUUUUCUUCAGCA |
| siRNA 1880 | 1880 | GCUGAAGAAAAAAACAACU | 7370 | AGUUGUUUUUUUCUUCAGC |
| siRNA 1881 | 1881 | CUGAAGAAAAAAACAACUG | 7371 | CAGUUGUUUUUUUCUUCAG |
| siRNA 1882 | 1882 | UGAAGAAAAAAACAACUGG | 7372 | CCAGUUGUUUUUUUCUUCA |
| siRNA 1883 | 1883 | GAAGAAAAAAACAACUGGA | 7373 | UCCAGUUGUUUUUUUCUUC |
| siRNA 1884 | 1884 | AAGAAAAAAACAACUGGAU | 7374 | AUCCAGUUGUUUUUUUCUU |
| siRNA 1885 | 1885 | AGAAAAAAACAACUGGAUG | 7375 | CAUCCAGUUGUUUUUUUCU |
| siRNA 1886 | 1886 | GAAAAAAACAACUGGAUGG | 7376 | CCAUCCAGUUGUUUUUUUC |
| siRNA 1887 | 1887 | AAAAAAACAACUGGAUGGC | 7377 | GCCAUCCAGUUGUUUUUUU |
| siRNA 1888 | 1888 | AAAAAACAACUGGAUGGCA | 7378 | UGCCAUCCAGUUGUUUUUU |
| siRNA 1889 | 1889 | AAAAACAACUGGAUGGCAG | 7379 | CUGCCAUCCAGUUGUUUUU |
| siRNA 1890 | 1890 | AAAACAACUGGAUGGCAGC | 7380 | GCUGCCAUCCAGUUGUUUU |
| siRNA 1891 | 1891 | AAACAACUGGAUGGCAGCC | 7381 | GGCUGCCAUCCAGUUGUUU |
| siRNA 1892 | 1892 | AACAACUGGAUGGCAGCCC | 7382 | GGGCUGCCAUCCAGUUGUU |
| siRNA 1893 | 1893 | ACAACUGGAUGGCAGCCCU | 7383 | AGGGCUGCCAUCCAGUUGU |
| siRNA 1894 | 1894 | CAACUGGAUGGCAGCCCUU | 7384 | AAGGGCUGCCAUCCAGUUG |
| siRNA 1895 | 1895 | AACUGGAUGGCAGCCCUUA | 7385 | UAAGGGCUGCCAUCCAGUU |
| siRNA 1896 | 1896 | ACUGGAUGGCAGCCCUUAU | 7386 | AUAAGGGCUGCCAUCCAGU |
| siRNA 1897 | 1897 | CUGGAUGGCAGCCCUUAUU | 7387 | AAUAAGGGCUGCCAUCCAG |
| siRNA 1898 | 1898 | UGGAUGGCAGCCCUUAUUU | 7388 | AAAUAAGGGCUGCCAUCCA |
| siRNA 1899 | 1899 | GGAUGGCAGCCCUUAUUUC | 7389 | GAAAUAAGGGCUGCCAUCC |
| siRNA 1900 | 1900 | GAUGGCAGCCCUUAUUUCU | 7390 | AGAAAUAAGGGCUGCCAUC |
| siRNA 1901 | 1901 | AUGGCAGCCCUUAUUUCUC | 7391 | GAGAAAUAAGGGCUGCCAU |
| siRNA 1902 | 1902 | UGGCAGCCCUUAUUUCUCU | 7392 | AGAGAAAUAAGGGCUGCCA |
| siRNA 1903 | 1903 | GGCAGCCCUUAUUUCUCUU | 7393 | AAGAGAAAUAAGGGCUGCC |
| siRNA 1904 | 1904 | GCAGCCCUUAUUUCUCUUC | 7394 | GAAGAGAAAUAAGGGCUGC |
| siRNA 1905 | 1905 | CAGCCCUUAUUUCUCUUCA | 7395 | UGAAGAGAAAUAAGGGCUG |
| siRNA 1906 | 1906 | AGCCCUUAUUUCUCUUCAU | 7396 | AUGAAGAGAAAUAAGGGCU |
| siRNA 1907 | 1907 | GCCCUUAUUUCUCUUCAUU | 7397 | AAUGAAGAGAAAUAAGGGC |
| siRNA 1908 | 1908 | CCCUUAUUUCUCUUCAUUA | 7398 | UAAUGAAGAGAAAUAAGGG |
| siRNA 1909 | 1909 | CCUUAUUUCUCUUCAUUAU | 7399 | AUAAUGAAGAGAAAUAAGG |
| siRNA 1910 | 1910 | CUUAUUUCUCUUCAUUAUC | 7400 | GAUAAUGAAGAGAAAUAAG |
| siRNA 1911 | 1911 | UUAUUUCUCUUCAUUAUCG | 7401 | CGAUAAUGAAGAGAAAUAA |
| siRNA 1912 | 1912 | UAUUUCUCUUCAUUAUCGU | 7402 | ACGAUAAUGAAGAGAAAUA |
| siRNA 1913 | 1913 | AUUUCUCUUCAUUAUCGUA | 7403 | UACGAUAAUGAAGAGAAAU |
| siRNA 1914 | 1914 | UUUCUCUUCAUUAUCGUAG | 7404 | CUACGAUAAUGAAGAGAAA |
| siRNA 1915 | 1915 | UUCUCUUCAUUAUCGUAGU | 7405 | ACUACGAUAAUGAAGAGAA |
| siRNA 1916 | 1916 | UCUCUUCAUUAUCGUAGUA | 7406 | UACUACGAUAAUGAAGAGA |
| siRNA 1917 | 1917 | CUCUUCAUUAUCGUAGUAC | 7407 | GUACUACGAUAAUGAAGAG |
| siRNA 1918 | 1918 | UCUUCAUUAUCGUAGUACU | 7408 | AGUACUACGAUAAUGAAGA |
| siRNA 1919 | 1919 | CUUCAUUAUCGUAGUACUC | 7409 | GAGUACUACGAUAAUGAAG |
| siRNA 1920 | 1920 | UUCAUUAUCGUAGUACUCU | 7410 | AGAGUACUACGAUAAUGAA |
| siRNA 1921 | 1921 | UCAUUAUCGUAGUACUCUA | 7411 | UAGAGUACUACGAUAAUGA |
| siRNA 1922 | 1922 | CAUUAUCGUAGUACUCUAG | 7412 | CUAGAGUACUACGAUAAUG |
| siRNA 1923 | 1923 | AUUAUCGUAGUACUCUAGA | 7413 | UCUAGAGUACUACGAUAAU |
| siRNA 1924 | 1924 | UUAUCGUAGUACUCUAGAU | 7414 | AUCUAGAGUACUACGAUAA |
| siRNA 1925 | 1925 | UAUCGUAGUACUCUAGAUC | 7415 | GAUCUAGAGUACUACGAUA |
| siRNA 1926 | 1926 | AUCGUAGUACUCUAGAUCG | 7416 | CGAUCUAGAGUACUACGAU |
| siRNA 1927 | 1927 | UCGUAGUACUCUAGAUCGA | 7417 | UCGAUCUAGAGUACUACGA |
| siRNA 1928 | 1928 | CGUAGUACUCUAGAUCGAA | 7418 | UUCGAUCUAGAGUACUACG |
| siRNA 1929 | 1929 | GUAGUACUCUAGAUCGAAU | 7419 | AUUCGAUCUAGAGUACUAC |
| siRNA 1930 | 1930 | UAGUACUCUAGAUCGAAUG | 7420 | CAUUCGAUCUAGAGUACUA |
| siRNA 1931 | 1931 | AGUACUCUAGAUCGAAUGU | 7421 | ACAUUCGAUCUAGAGUACU |
| siRNA 1932 | 1932 | GUACUCUAGAUCGAAUGUU | 7422 | AACAUUCGAUCUAGAGUAC |
| siRNA 1933 | 1933 | UACUCUAGAUCGAAUGUUA | 7423 | UAACAUUCGAUCUAGAGUA |
| siRNA 1934 | 1934 | ACUCUAGAUCGAAUGUUAG | 7424 | CUAACAUUCGAUCUAGAGU |
| siRNA 1935 | 1935 | CUCUAGAUCGAAUGUUAGA | 7425 | UCUAACAUUCGAUCUAGAG |
| siRNA 1936 | 1936 | UCUAGAUCGAAUGUUAGAU | 7426 | AUCUAACAUUCGAUCUAGA |
| siRNA 1937 | 1937 | CUAGAUCGAAUGUUAGAUU | 7427 | AAUCUAACAUUCGAUCUAG |
| siRNA 1938 | 1938 | UAGAUCGAAUGUUAGAUUC | 7428 | GAAUCUAACAUUCGAUCUA |
| siRNA 1939 | 1939 | AGAUCGAAUGUUAGAUUCA | 7429 | UGAAUCUAACAUUCGAUCU |
| siRNA 1940 | 1940 | GAUCGAAUGUUAGAUUCAG | 7430 | CUGAAUCUAACAUUCGAUC |
| siRNA 1941 | 1941 | AUCGAAUGUUAGAUUCAGU | 7431 | ACUGAAUCUAACAUUCGAU |
| siRNA 1942 | 1942 | UCGAAUGUUAGAUUCAGUA | 7432 | UACUGAAUCUAACAUUCGA |
| siRNA 1943 | 1943 | CGAAUGUUAGAUUCAGUAU | 7433 | AUACUGAAUCUAACAUUCG |
| siRNA 1944 | 1944 | GAAUGUUAGAUUCAGUAUU | 7434 | AAUACUGAAUCUAACAUUC |
| siRNA 1945 | 1945 | AAUGUUAGAUUCAGUAUUA | 7435 | UAAUACUGAAUCUAACAUU |
| siRNA 1946 | 1946 | AUGUUAGAUUCAGUAUUAU | 7436 | AUAAUACUGAAUCUAACAU |
| siRNA 1947 | 1947 | UGUUAGAUUCAGUAUUAUU | 7437 | AAUAAUACUGAAUCUAACA |
| siRNA 1948 | 1948 | GUUAGAUUCAGUAUUAUUG | 7438 | CAAUAAUACUGAAUCUAAC |
| siRNA 1949 | 1949 | UUAGAUUCAGUAUUAUUGA | 7439 | UCAAUAAUACUGAAUCUAA |
| siRNA 1950 | 1950 | UAGAUUCAGUAUUAUUGAA | 7440 | UUCAAUAAUACUGAAUCUA |
| siRNA 1951 | 1951 | AGAUUCAGUAUUAUUGAAA | 7441 | UUUCAAUAAUACUGAAUCU |
| siRNA 1952 | 1952 | GAUUCAGUAUUAUUGAAAG | 7442 | CUUUCAAUAAUACUGAAUC |
| siRNA 1953 | 1953 | AUUCAGUAUUAUUGAAAGA | 7443 | UCUUUCAAUAAUACUGAAU |
| siRNA 1954 | 1954 | UUCAGUAUUAUUGAAAGAA | 7444 | UUCUUUCAAUAAUACUGAA |
| siRNA 1955 | 1955 | UCAGUAUUAUUGAAAGAAG | 7445 | CUUCUUUCAAUAAUACUGA |
| siRNA 1956 | 1956 | CAGUAUUAUUGAAAGAAGA | 7446 | UCUUCUUUCAAUAAUACUG |
| siRNA 1957 | 1957 | AGUAUUAUUGAAAGAAGAA | 7447 | UUCUUCUUUCAAUAAUACU |
| siRNA 1958 | 1958 | GUAUUAUUGAAAGAAGAAA | 7448 | UUUCUUCUUUCAAUAAUAC |
| siRNA 1959 | 1959 | UAUUAUUGAAAGAAGAAAA | 7449 | UUUUCUUCUUUCAAUAAUA |
| siRNA 1960 | 1960 | AUUAUUGAAAGAAGAAAAU | 7450 | AUUUUCUUCUUUCAAUAAU |
| siRNA 1961 | 1961 | UUAUUGAAAGAAGAAAAUG | 7451 | CAUUUUCUUCUUUCAAUAA |
| siRNA 1962 | 1962 | UAUUGAAAGAAGAAAAUGA | 7452 | UCAUUUUCUUCUUUCAAUA |
| siRNA 1963 | 1963 | AUUGAAAGAAGAAAAUGAG | 7453 | CUCAUUUUCUUCUUUCAAU |
| siRNA 1964 | 1964 | UUGAAAGAAGAAAAUGAGC | 7454 | GCUCAUUUUCUUCUUUCAA |
| siRNA 1965 | 1965 | UGAAAGAAGAAAAUGAGCA | 7455 | UGCUCAUUUUCUUCUUUCA |
| siRNA 1966 | 1966 | GAAAGAAGAAAAUGAGCAA | 7456 | UUGCUCAUUUUCUUCUUUC |
| siRNA 1967 | 1967 | AAAGAAGAAAAUGAGCAAC | 7457 | GUUGCUCAUUUUCUUCUUU |
| siRNA 1968 | 1968 | AAGAAGAAAAUGAGCAACC | 7458 | GGUUGCUCAUUUUCUUCUU |
| siRNA 1969 | 1969 | AGAAGAAAAUGAGCAACCA | 7459 | UGGUUGCUCAUUUUCUUCU |
| siRNA 1970 | 1970 | GAAGAAAAUGAGCAACCAC | 7460 | GUGGUUGCUCAUUUUCUUC |
| siRNA 1971 | 1971 | AAGAAAAUGAGCAACCACU | 7461 | AGUGGUUGCUCAUUUUCUU |
| siRNA 1972 | 1972 | AGAAAAUGAGCAACCACUG | 7462 | CAGUGGUUGCUCAUUUUCU |
| siRNA 1973 | 1973 | GAAAAUGAGCAACCACUGA | 7463 | UCAGUGGUUGCUCAUUUUC |
| siRNA 1974 | 1974 | AAAAUGAGCAACCACUGAG | 7464 | CUCAGUGGUUGCUCAUUUU |
| siRNA 1975 | 1975 | AAAUGAGCAACCACUGAGA | 7465 | UCUCAGUGGUUGCUCAUUU |
| siRNA 1976 | 1976 | AAUGAGCAACCACUGAGAU | 7466 | AUCUCAGUGGUUGCUCAUU |
| siRNA 1977 | 1977 | AUGAGCAACCACUGAGAUU | 7467 | AAUCUCAGUGGUUGCUCAU |
| siRNA 1978 | 1978 | UGAGCAACCACUGAGAUUA | 7468 | UAAUCUCAGUGGUUGCUCA |
| siRNA 1979 | 1979 | GAGCAACCACUGAGAUUAC | 7469 | GUAAUCUCAGUGGUUGCUC |
| siRNA 1980 | 1980 | AGCAACCACUGAGAUUACC | 7470 | GGUAAUCUCAGUGGUUGCU |
| siRNA 1981 | 1981 | GCAACCACUGAGAUUACCA | 7471 | UGGUAAUCUCAGUGGUUGC |
| siRNA 1982 | 1982 | CAACCACUGAGAUUACCAA | 7472 | UUGGUAAUCUCAGUGGUUG |
| siRNA 1983 | 1983 | AACCACUGAGAUUACCAAG | 7473 | CUUGGUAAUCUCAGUGGUU |
| siRNA 1984 | 1984 | ACCACUGAGAUUACCAAGU | 7474 | ACUUGGUAAUCUCAGUGGU |
| siRNA 1985 | 1985 | CCACUGAGAUUACCAAGUC | 7475 | GACUUGGUAAUCUCAGUGG |
| siRNA 1986 | 1986 | CACUGAGAUUACCAAGUCC | 7476 | GGACUUGGUAAUCUCAGUG |
| siRNA 1987 | 1987 | ACUGAGAUUACCAAGUCCU | 7477 | AGGACUUGGUAAUCUCAGU |
| siRNA 1988 | 1988 | CUGAGAUUACCAAGUCCUG | 7478 | CAGGACUUGGUAAUCUCAG |
| siRNA 1989 | 1989 | UGAGAUUACCAAGUCCUGA | 7479 | UCAGGACUUGGUAAUCUCA |
| siRNA 1990 | 1990 | GAGAUUACCAAGUCCUGAA | 7480 | UUCAGGACUUGGUAAUCUC |
| siRNA 1991 | 1991 | AGAUUACCAAGUCCUGAAG | 7481 | CUUCAGGACUUGGUAAUCU |
| siRNA 1992 | 1992 | GAUUACCAAGUCCUGAAGU | 7482 | ACUUCAGGACUUGGUAAUC |
| siRNA 1993 | 1993 | AUUACCAAGUCCUGAAGUA | 7483 | UACUUCAGGACUUGGUAAU |
| siRNA 1994 | 1994 | UUACCAAGUCCUGAAGUAU | 7484 | AUACUUCAGGACUUGGUAA |
| siRNA 1995 | 1995 | UACCAAGUCCUGAAGUAUA | 7485 | UAUACUUCAGGACUUGGUA |
| siRNA 1996 | 1996 | ACCAAGUCCUGAAGUAUAU | 7486 | AUAUACUUCAGGACUUGGU |
| siRNA 1997 | 1997 | CCAAGUCCUGAAGUAUAUC | 7487 | GAUAUACUUCAGGACUUGG |
| siRNA 1998 | 1998 | CAAGUCCUGAAGUAUAUCG | 7488 | CGAUAUACUUCAGGACUUG |
| siRNA 1999 | 1999 | AAGUCCUGAAGUAUAUCGU | 7489 | ACGAUAUACUUCAGGACUU |
| siRNA 2000 | 2000 | AGUCCUGAAGUAUAUCGUU | 7490 | AACGAUAUACUUCAGGACU |
| siRNA 2001 | 2001 | GUCCUGAAGUAUAUCGUUU | 7491 | AAACGAUAUACUUCAGGAC |
| siRNA 2002 | 2002 | UCCUGAAGUAUAUCGUUUU | 7492 | AAAACGAUAUACUUCAGGA |
| siRNA 2003 | 2003 | CCUGAAGUAUAUCGUUUUG | 7493 | CAAAACGAUAUACUUCAGG |
| siRNA 2004 | 2004 | CUGAAGUAUAUCGUUUUGU | 7494 | ACAAAACGAUAUACUUCAG |
| siRNA 2005 | 2005 | UGAAGUAUAUCGUUUUGUA | 7495 | UACAAAACGAUAUACUUCA |
| siRNA 2006 | 2006 | GAAGUAUAUCGUUUUGUAG | 7496 | CUACAAAACGAUAUACUUC |
| siRNA 2007 | 2007 | AAGUAUAUCGUUUUGUAGU | 7497 | ACUACAAAACGAUAUACUU |
| siRNA 2008 | 2008 | AGUAUAUCGUUUUGUAGUA | 7498 | UACUACAAAACGAUAUACU |
| siRNA 2009 | 2009 | GUAUAUCGUUUUGUAGUAA | 7499 | UUACUACAAAACGAUAUAC |
| siRNA 2010 | 2010 | UAUAUCGUUUUGUAGUAAA | 7500 | UUUACUACAAAACGAUAUA |
| siRNA 2011 | 2011 | AUAUCGUUUUGUAGUAAAA | 7501 | UUUUACUACAAAACGAUAU |
| siRNA 2012 | 2012 | UAUCGUUUUGUAGUAAAAG | 7502 | CUUUUACUACAAAACGAUA |
| siRNA 2013 | 2013 | AUCGUUUUGUAGUAAAAGA | 7503 | UCUUUUACUACAAAACGAU |
| siRNA 2014 | 2014 | UCGUUUUGUAGUAAAAGAC | 7504 | GUCUUUUACUACAAAACGA |
| siRNA 2015 | 2015 | CGUUUUGUAGUAAAAGACU | 7505 | AGUCUUUUACUACAAAACG |
| siRNA 2016 | 2016 | GUUUUGUAGUAAAAGACUC | 7506 | GAGUCUUUUACUACAAAAC |
| siRNA 2017 | 2017 | UUUUGUAGUAAAAGACUCU | 7507 | AGAGUCUUUUACUACAAAA |
| siRNA 2018 | 2018 | UUUGUAGUAAAAGACUCUG | 7508 | CAGAGUCUUUUACUACAAA |
| siRNA 2019 | 2019 | UUGUAGUAAAAGACUCUGA | 7509 | UCAGAGUCUUUUACUACAA |
| siRNA 2020 | 2020 | UGUAGUAAAAGACUCUGAG | 7510 | CUCAGAGUCUUUUACUACA |
| siRNA 2021 | 2021 | GUAGUAAAAGACUCUGAGG | 7511 | CCUCAGAGUCUUUUACUAC |
| siRNA 2022 | 2022 | UAGUAAAAGACUCUGAGGA | 7512 | UCCUCAGAGUCUUUUACUA |
| siRNA 2023 | 2023 | AGUAAAAGACUCUGAGGAA | 7513 | UUCCUCAGAGUCUUUUACU |
| siRNA 2024 | 2024 | GUAAAAGACUCUGAGGAAA | 7514 | UUUCCUCAGAGUCUUUUAC |
| siRNA 2025 | 2025 | UAAAAGACUCUGAGGAAAA | 7515 | UUUUCCUCAGAGUCUUUUA |
| siRNA 2026 | 2026 | AAAAGACUCUGAGGAAAAC | 7516 | GUUUUCCUCAGAGUCUUUU |
| siRNA 2027 | 2027 | AAAGACUCUGAGGAAAACA | 7517 | UGUUUUCCUCAGAGUCUUU |
| siRNA 2028 | 2028 | AAGACUCUGAGGAAAACAU | 7518 | AUGUUUUCCUCAGAGUCUU |
| siRNA 2029 | 2029 | AGACUCUGAGGAAAACAUU | 7519 | AAUGUUUUCCUCAGAGUCU |
| siRNA 2030 | 2030 | GACUCUGAGGAAAACAUUG | 7520 | CAAUGUUUUCCUCAGAGUC |
| siRNA 2031 | 2031 | ACUCUGAGGAAAACAUUGU | 7521 | ACAAUGUUUUCCUCAGAGU |
| siRNA 2032 | 2032 | CUCUGAGGAAAACAUUGUU | 7522 | AACAAUGUUUUCCUCAGAG |
| siRNA 2033 | 2033 | UCUGAGGAAAACAUUGUUU | 7523 | AAACAAUGUUUUCCUCAGA |
| siRNA 2034 | 2034 | CUGAGGAAAACAUUGUUUU | 7524 | AAAACAAUGUUUUCCUCAG |
| siRNA 2035 | 2035 | UGAGGAAAACAUUGUUUUU | 7525 | AAAAACAAUGUUUUCCUCA |
| siRNA 2036 | 2036 | GAGGAAAACAUUGUUUUUG | 7526 | CAAAAACAAUGUUUUCCUC |
| siRNA 2037 | 2037 | AGGAAAACAUUGUUUUUGA | 7527 | UCAAAAACAAUGUUUUCCU |
| siRNA 2038 | 2038 | GGAAAACAUUGUUUUUGAA | 7528 | UUCAAAAACAAUGUUUUCC |
| siRNA 2039 | 2039 | GAAAACAUUGUUUUUGAAG | 7529 | CUUCAAAAACAAUGUUUUC |
| siRNA 2040 | 2040 | AAAACAUUGUUUUUGAAGA | 7530 | UCUUCAAAAACAAUGUUUU |
| siRNA 2041 | 2041 | AAACAUUGUUUUUGAAGAC | 7531 | GUCUUCAAAAACAAUGUUU |
| siRNA 2042 | 2042 | AACAUUGUUUUUGAAGACA | 7532 | UGUCUUCAAAAACAAUGUU |
| siRNA 2043 | 2043 | ACAUUGUUUUUGAAGACAA | 7533 | UUGUCUUCAAAAACAAUGU |
| siRNA 2044 | 2044 | CAUUGUUUUUGAAGACAAC | 7534 | GUUGUCUUCAAAAACAAUG |
| siRNA 2045 | 2045 | AUUGUUUUUGAAGACAACU | 7535 | AGUUGUCUUCAAAAACAAU |
| siRNA 2046 | 2046 | UUGUUUUUGAAGACAACUU | 7536 | AAGUUGUCUUCAAAAACAA |
| siRNA 2047 | 2047 | UGUUUUUGAAGACAACUUG | 7537 | CAAGUUGUCUUCAAAAACA |
| siRNA 2048 | 2048 | GUUUUUGAAGACAACUUGC | 7538 | GCAAGUUGUCUUCAAAAAC |
| siRNA 2049 | 2049 | UUUUUGAAGACAACUUGCA | 7539 | UGCAAGUUGUCUUCAAAAA |
| siRNA 2050 | 2050 | UUUUGAAGACAACUUGCAA | 7540 | UUGCAAGUUGUCUUCAAAA |
| siRNA 2051 | 2051 | UUUGAAGACAACUUGCAAA | 7541 | UUUGCAAGUUGUCUUCAAA |
| siRNA 2052 | 2052 | UUGAAGACAACUUGCAAAG | 7542 | CUUUGCAAGUUGUCUUCAA |
| siRNA 2053 | 2053 | UGAAGACAACUUGCAAAGU | 7543 | ACUUUGCAAGUUGUCUUCA |
| siRNA 2054 | 2054 | GAAGACAACUUGCAAAGUA | 7544 | UACUUUGCAAGUUGUCUUC |
| siRNA 2055 | 2055 | AAGACAACUUGCAAAGUAG | 7545 | CUACUUUGCAAGUUGUCUU |
| siRNA 2056 | 2056 | AGACAACUUGCAAAGUAGA | 7546 | UCUACUUUGCAAGUUGUCU |
| siRNA 2057 | 2057 | GACAACUUGCAAAGUAGAA | 7547 | UUCUACUUUGCAAGUUGUC |
| siRNA 2058 | 2058 | ACAACUUGCAAAGUAGAAG | 7548 | CUUCUACUUUGCAAGUUGU |
| siRNA 2059 | 2059 | CAACUUGCAAAGUAGAAGU | 7549 | ACUUCUACUUUGCAAGUUG |
| siRNA 2060 | 2060 | AACUUGCAAAGUAGAAGUG | 7550 | CACUUCUACUUUGCAAGUU |
| siRNA 2061 | 2061 | ACUUGCAAAGUAGAAGUGG | 7551 | CCACUUCUACUUUGCAAGU |
| siRNA 2062 | 2062 | CUUGCAAAGUAGAAGUGGC | 7552 | GCCACUUCUACUUUGCAAG |
| siRNA 2063 | 2063 | UUGCAAAGUAGAAGUGGCA | 7553 | UGCCACUUCUACUUUGCAA |
| siRNA 2064 | 2064 | UGCAAAGUAGAAGUGGCAU | 7554 | AUGCCACUUCUACUUUGCA |
| siRNA 2065 | 2065 | GCAAAGUAGAAGUGGCAUC | 7555 | GAUGCCACUUCUACUUUGC |
| siRNA 2066 | 2066 | CAAAGUAGAAGUGGCAUCC | 7556 | GGAUGCCACUUCUACUUUG |
| siRNA 2067 | 2067 | AAAGUAGAAGUGGCAUCCC | 7557 | GGGAUGCCACUUCUACUUU |
| siRNA 2068 | 2068 | AAGUAGAAGUGGCAUCCCC | 7558 | GGGGAUGCCACUUCUACUU |
| siRNA 2069 | 2069 | AGUAGAAGUGGCAUCCCCA | 7559 | UGGGGAUGCCACUUCUACU |
| siRNA 2070 | 2070 | GUAGAAGUGGCAUCCCCAU | 7560 | AUGGGGAUGCCACUUCUAC |
| siRNA 2071 | 2071 | UAGAAGUGGCAUCCCCAUU | 7561 | AAUGGGGAUGCCACUUCUA |
| siRNA 2072 | 2072 | AGAAGUGGCAUCCCCAUUA | 7562 | UAAUGGGGAUGCCACUUCU |
| siRNA 2073 | 2073 | GAAGUGGCAUCCCCAUUAU | 7563 | AUAAUGGGGAUGCCACUUC |
| siRNA 2074 | 2074 | AAGUGGCAUCCCCAUUAUU | 7564 | AAUAAUGGGGAUGCCACUU |
| siRNA 2075 | 2075 | AGUGGCAUCCCCAUUAUUA | 7565 | UAAUAAUGGGGAUGCCACU |
| siRNA 2076 | 2076 | GUGGCAUCCCCAUUAUUAA | 7566 | UUAAUAAUGGGGAUGCCAC |
| siRNA 2077 | 2077 | UGGCAUCCCCAUUAUUAAA | 7567 | UUUAAUAAUGGGGAUGCCA |
| siRNA 2078 | 2078 | GGCAUCCCCAUUAUUAAAG | 7568 | CUUUAAUAAUGGGGAUGCC |
| siRNA 2079 | 2079 | GCAUCCCCAUUAUUAAAGG | 7569 | CCUUUAAUAAUGGGGAUGC |
| siRNA 2080 | 2080 | CAUCCCCAUUAUUAAAGGA | 7570 | UCCUUUAAUAAUGGGGAUG |
| siRNA 2081 | 2081 | AUCCCCAUUAUUAAAGGAG | 7571 | CUCCUUUAAUAAUGGGGAU |
| siRNA 2082 | 2082 | UCCCCAUUAUUAAAGGAGG | 7572 | CCUCCUUUAAUAAUGGGGA |
| siRNA 2083 | 2083 | CCCCAUUAUUAAAGGAGGA | 7573 | UCCUCCUUUAAUAAUGGGG |
| siRNA 2084 | 2084 | CCCAUUAUUAAAGGAGGAA | 7574 | UUCCUCCUUUAAUAAUGGG |
| siRNA 2085 | 2085 | CCAUUAUUAAAGGAGGAAC | 7575 | GUUCCUCCUUUAAUAAUGG |
| siRNA 2086 | 2086 | CAUUAUUAAAGGAGGAACU | 7576 | AGUUCCUCCUUUAAUAAUG |
| siRNA 2087 | 2087 | AUUAUUAAAGGAGGAACUG | 7577 | CAGUUCCUCCUUUAAUAAU |
| siRNA 2088 | 2088 | UUAUUAAAGGAGGAACUGU | 7578 | ACAGUUCCUCCUUUAAUAA |
| siRNA 2089 | 2089 | UAUUAAAGGAGGAACUGUA | 7579 | UACAGUUCCUCCUUUAAUA |
| siRNA 2090 | 2090 | AUUAAAGGAGGAACUGUAG | 7580 | CUACAGUUCCUCCUUUAAU |
| siRNA 2091 | 2091 | UUAAAGGAGGAACUGUAGU | 7581 | ACUACAGUUCCUCCUUUAA |
| siRNA 2092 | 2092 | UAAAGGAGGAACUGUAGUG | 7582 | CACUACAGUUCCUCCUUUA |
| siRNA 2093 | 2093 | AAAGGAGGAACUGUAGUGA | 7583 | UCACUACAGUUCCUCCUUU |
| siRNA 2094 | 2094 | AAGGAGGAACUGUAGUGAA | 7584 | UUCACUACAGUUCCUCCUU |
| siRNA 2095 | 2095 | AGGAGGAACUGUAGUGAAA | 7585 | UUUCACUACAGUUCCUCCU |
| siRNA 2096 | 2096 | GGAGGAACUGUAGUGAAAU | 7586 | AUUUCACUACAGUUCCUCC |
| siRNA 2097 | 2097 | GAGGAACUGUAGUGAAAUU | 7587 | AAUUUCACUACAGUUCCUC |
| siRNA 2098 | 2098 | AGGAACUGUAGUGAAAUUA | 7588 | UAAUUUCACUACAGUUCCU |
| siRNA 2099 | 2099 | GGAACUGUAGUGAAAUUAA | 7589 | UUAAUUUCACUACAGUUCC |
| siRNA 2100 | 2100 | GAACUGUAGUGAAAUUAAU | 7590 | AUUAAUUUCACUACAGUUC |
| siRNA 2101 | 2101 | AACUGUAGUGAAAUUAAUU | 7591 | AAUUAAUUUCACUACAGUU |
| siRNA 2102 | 2102 | ACUGUAGUGAAAUUAAUUG | 7592 | CAAUUAAUUUCACUACAGU |
| siRNA 2103 | 2103 | CUGUAGUGAAAUUAAUUGA | 7593 | UCAAUUAAUUUCACUACAG |
| siRNA 2104 | 2104 | UGUAGUGAAAUUAAUUGAA | 7594 | UUCAAUUAAUUUCACUACA |
| siRNA 2105 | 2105 | GUAGUGAAAUUAAUUGAAA | 7595 | UUUCAAUUAAUUUCACUAC |
| siRNA 2106 | 2106 | UAGUGAAAUUAAUUGAAAG | 7596 | CUUUCAAUUAAUUUCACUA |
| siRNA 2107 | 2107 | AGUGAAAUUAAUUGAAAGG | 7597 | CCUUUCAAUUAAUUUCACU |
| siRNA 2108 | 2108 | GUGAAAUUAAUUGAAAGGU | 7598 | ACCUUUCAAUUAAUUUCAC |
| siRNA 2109 | 2109 | UGAAAUUAAUUGAAAGGUU | 7599 | AACCUUUCAAUUAAUUUCA |
| siRNA 2110 | 2110 | GAAAUUAAUUGAAAGGUUA | 7600 | UAACCUUUCAAUUAAUUUC |
| siRNA 2111 | 2111 | AAAUUAAUUGAAAGGUUAA | 7601 | UUAACCUUUCAAUUAAUUU |
| siRNA 2112 | 2112 | AAUUAAUUGAAAGGUUAAC | 7602 | GUUAACCUUUCAAUUAAUU |
| siRNA 2113 | 2113 | AUUAAUUGAAAGGUUAACA | 7603 | UGUUAACCUUUCAAUUAAU |
| siRNA 2114 | 2114 | UUAAUUGAAAGGUUAACAU | 7604 | AUGUUAACCUUUCAAUUAA |
| siRNA 2115 | 2115 | UAAUUGAAAGGUUAACAUA | 7605 | UAUGUUAACCUUUCAAUUA |
| siRNA 2116 | 2116 | AAUUGAAAGGUUAACAUAU | 7606 | AUAUGUUAACCUUUCAAUU |
| siRNA 2117 | 2117 | AUUGAAAGGUUAACAUAUC | 7607 | GAUAUGUUAACCUUUCAAU |
| siRNA 2118 | 2118 | UUGAAAGGUUAACAUAUCA | 7608 | UGAUAUGUUAACCUUUCAA |
| siRNA 2119 | 2119 | UGAAAGGUUAACAUAUCAU | 7609 | AUGAUAUGUUAACCUUUCA |
| siRNA 2120 | 2120 | GAAAGGUUAACAUAUCAUA | 7610 | UAUGAUAUGUUAACCUUUC |
| siRNA 2121 | 2121 | AAAGGUUAACAUAUCAUAU | 7611 | AUAUGAUAUGUUAACCUUU |
| siRNA 2122 | 2122 | AAGGUUAACAUAUCAUAUG | 7612 | CAUAUGAUAUGUUAACCUU |
| siRNA 2123 | 2123 | AGGUUAACAUAUCAUAUGU | 7613 | ACAUAUGAUAUGUUAACCU |
| siRNA 2124 | 2124 | GGUUAACAUAUCAUAUGUA | 7614 | UACAUAUGAUAUGUUAACC |
| siRNA 2125 | 2125 | GUUAACAUAUCAUAUGUAU | 7615 | AUACAUAUGAUAUGUUAAC |
| siRNA 2126 | 2126 | UUAACAUAUCAUAUGUAUG | 7616 | CAUACAUAUGAUAUGUUAA |
| siRNA 2127 | 2127 | UAACAUAUCAUAUGUAUGC | 7617 | GCAUACAUAUGAUAUGUUA |
| siRNA 2128 | 2128 | AACAUAUCAUAUGUAUGCA | 7618 | UGCAUACAUAUGAUAUGUU |
| siRNA 2129 | 2129 | ACAUAUCAUAUGUAUGCAG | 7619 | CUGCAUACAUAUGAUAUGU |
| siRNA 2130 | 2130 | CAUAUCAUAUGUAUGCAGA | 7620 | UCUGCAUACAUAUGAUAUG |
| siRNA 2131 | 2131 | AUAUCAUAUGUAUGCAGAU | 7621 | AUCUGCAUACAUAUGAUAU |
| siRNA 2132 | 2132 | UAUCAUAUGUAUGCAGAUC | 7622 | GAUCUGCAUACAUAUGAUA |
| siRNA 2133 | 2133 | AUCAUAUGUAUGCAGAUCC | 7623 | GGAUCUGCAUACAUAUGAU |
| siRNA 2134 | 2134 | UCAUAUGUAUGCAGAUCCC | 7624 | GGGAUCUGCAUACAUAUGA |
| siRNA 2135 | 2135 | CAUAUGUAUGCAGAUCCCA | 7625 | UGGGAUCUGCAUACAUAUG |
| siRNA 2136 | 2136 | AUAUGUAUGCAGAUCCCAA | 7626 | UUGGGAUCUGCAUACAUAU |
| siRNA 2137 | 2137 | UAUGUAUGCAGAUCCCAAU | 7627 | AUUGGGAUCUGCAUACAUA |
| siRNA 2138 | 2138 | AUGUAUGCAGAUCCCAAUU | 7628 | AAUUGGGAUCUGCAUACAU |
| siRNA 2139 | 2139 | UGUAUGCAGAUCCCAAUUU | 7629 | AAAUUGGGAUCUGCAUACA |
| siRNA 2140 | 2140 | GUAUGCAGAUCCCAAUUUU | 7630 | AAAAUUGGGAUCUGCAUAC |
| siRNA 2141 | 2141 | UAUGCAGAUCCCAAUUUUG | 7631 | CAAAAUUGGGAUCUGCAUA |
| siRNA 2142 | 2142 | AUGCAGAUCCCAAUUUUGU | 7632 | ACAAAAUUGGGAUCUGCAU |
| siRNA 2143 | 2143 | UGCAGAUCCCAAUUUUGUU | 7633 | AACAAAAUUGGGAUCUGCA |
| siRNA 2144 | 2144 | GCAGAUCCCAAUUUUGUUC | 7634 | GAACAAAAUUGGGAUCUGC |
| siRNA 2145 | 2145 | CAGAUCCCAAUUUUGUUCG | 7635 | CGAACAAAAUUGGGAUCUG |
| siRNA 2146 | 2146 | AGAUCCCAAUUUUGUUCGU | 7636 | ACGAACAAAAUUGGGAUCU |
| siRNA 2147 | 2147 | GAUCCCAAUUUUGUUCGUA | 7637 | UACGAACAAAAUUGGGAUC |
| siRNA 2148 | 2148 | AUCCCAAUUUUGUUCGUAC | 7638 | GUACGAACAAAAUUGGGAU |
| siRNA 2149 | 2149 | UCCCAAUUUUGUUCGUACU | 7639 | AGUACGAACAAAAUUGGGA |
| siRNA 2150 | 2150 | CCCAAUUUUGUUCGUACUU | 7640 | AAGUACGAACAAAAUUGGG |
| siRNA 2151 | 2151 | CCAAUUUUGUUCGUACUUU | 7641 | AAAGUACGAACAAAAUUGG |
| siRNA 2152 | 2152 | CAAUUUUGUUCGUACUUUU | 7642 | AAAAGUACGAACAAAAUUG |
| siRNA 2153 | 2153 | AAUUUUGUUCGUACUUUUC | 7643 | GAAAAGUACGAACAAAAUU |
| siRNA 2154 | 2154 | AUUUUGUUCGUACUUUUCU | 7644 | AGAAAAGUACGAACAAAAU |
| siRNA 2155 | 2155 | UUUUGUUCGUACUUUUCUU | 7645 | AAGAAAAGUACGAACAAAA |
| siRNA 2156 | 2156 | UUUGUUCGUACUUUUCUUA | 7646 | UAAGAAAAGUACGAACAAA |
| siRNA 2157 | 2157 | UUGUUCGUACUUUUCUUAC | 7647 | GUAAGAAAAGUACGAACAA |
| siRNA 2158 | 2158 | UGUUCGUACUUUUCUUACC | 7648 | GGUAAGAAAAGUACGAACA |
| siRNA 2159 | 2159 | GUUCGUACUUUUCUUACCA | 7649 | UGGUAAGAAAAGUACGAAC |
| siRNA 2160 | 2160 | UUCGUACUUUUCUUACCAC | 7650 | GUGGUAAGAAAAGUACGAA |
| siRNA 2161 | 2161 | UCGUACUUUUCUUACCACA | 7651 | UGUGGUAAGAAAAGUACGA |
| siRNA 2162 | 2162 | CGUACUUUUCUUACCACAU | 7652 | AUGUGGUAAGAAAAGUACG |
| siRNA 2163 | 2163 | GUACUUUUCUUACCACAUA | 7653 | UAUGUGGUAAGAAAAGUAC |
| siRNA 2164 | 2164 | UACUUUUCUUACCACAUAU | 7654 | AUAUGUGGUAAGAAAAGUA |
| siRNA 2165 | 2165 | ACUUUUCUUACCACAUAUC | 7655 | GAUAUGUGGUAAGAAAAGU |
| siRNA 2166 | 2166 | CUUUUCUUACCACAUAUCG | 7656 | CGAUAUGUGGUAAGAAAAG |
| siRNA 2167 | 2167 | UUUUCUUACCACAUAUCGU | 7657 | ACGAUAUGUGGUAAGAAAA |
| siRNA 2168 | 2168 | UUUCUUACCACAUAUCGUU | 7658 | AACGAUAUGUGGUAAGAAA |
| siRNA 2169 | 2169 | UUCUUACCACAUAUCGUUC | 7659 | GAACGAUAUGUGGUAAGAA |
| siRNA 2170 | 2170 | UCUUACCACAUAUCGUUCA | 7660 | UGAACGAUAUGUGGUAAGA |
| siRNA 2171 | 2171 | CUUACCACAUAUCGUUCAU | 7661 | AUGAACGAUAUGUGGUAAG |
| siRNA 2172 | 2172 | UUACCACAUAUCGUUCAUU | 7662 | AAUGAACGAUAUGUGGUAA |
| siRNA 2173 | 2173 | UACCACAUAUCGUUCAUUU | 7663 | AAAUGAACGAUAUGUGGUA |
| siRNA 2174 | 2174 | ACCACAUAUCGUUCAUUUU | 7664 | AAAAUGAACGAUAUGUGGU |
| siRNA 2175 | 2175 | CCACAUAUCGUUCAUUUUG | 7665 | CAAAAUGAACGAUAUGUGG |
| siRNA 2176 | 2176 | CACAUAUCGUUCAUUUUGU | 7666 | ACAAAAUGAACGAUAUGUG |
| siRNA 2177 | 2177 | ACAUAUCGUUCAUUUUGUA | 7667 | UACAAAAUGAACGAUAUGU |
| siRNA 2178 | 2178 | CAUAUCGUUCAUUUUGUAA | 7668 | UUACAAAAUGAACGAUAUG |
| siRNA 2179 | 2179 | AUAUCGUUCAUUUUGUAAA | 7669 | UUUACAAAAUGAACGAUAU |
| siRNA 2180 | 2180 | UAUCGUUCAUUUUGUAAAC | 7670 | GUUUACAAAAUGAACGAUA |
| siRNA 2181 | 2181 | AUCGUUCAUUUUGUAAACC | 7671 | GGUUUACAAAAUGAACGAU |
| siRNA 2182 | 2182 | UCGUUCAUUUUGUAAACCA | 7672 | UGGUUUACAAAAUGAACGA |
| siRNA 2183 | 2183 | CGUUCAUUUUGUAAACCAC | 7673 | GUGGUUUACAAAAUGAACG |
| siRNA 2184 | 2184 | GUUCAUUUUGUAAACCACA | 7674 | UGUGGUUUACAAAAUGAAC |
| siRNA 2185 | 2185 | UUCAUUUUGUAAACCACAG | 7675 | CUGUGGUUUACAAAAUGAA |
| siRNA 2186 | 2186 | UCAUUUUGUAAACCACAGG | 7676 | CCUGUGGUUUACAAAAUGA |
| siRNA 2187 | 2187 | CAUUUUGUAAACCACAGGA | 7677 | UCCUGUGGUUUACAAAAUG |
| siRNA 2188 | 2188 | AUUUUGUAAACCACAGGAA | 7678 | UUCCUGUGGUUUACAAAAU |
| siRNA 2189 | 2189 | UUUUGUAAACCACAGGAAU | 7679 | AUUCCUGUGGUUUACAAAA |
| siRNA 2190 | 2190 | UUUGUAAACCACAGGAAUU | 7680 | AAUUCCUGUGGUUUACAAA |
| siRNA 2191 | 2191 | UUGUAAACCACAGGAAUUG | 7681 | CAAUUCCUGUGGUUUACAA |
| siRNA 2192 | 2192 | UGUAAACCACAGGAAUUGC | 7682 | GCAAUUCCUGUGGUUUACA |
| siRNA 2193 | 2193 | GUAAACCACAGGAAUUGCU | 7683 | AGCAAUUCCUGUGGUUUAC |
| siRNA 2194 | 2194 | UAAACCACAGGAAUUGCUG | 7684 | CAGCAAUUCCUGUGGUUUA |
| siRNA 2195 | 2195 | AAACCACAGGAAUUGCUGA | 7685 | UCAGCAAUUCCUGUGGUUU |
| siRNA 2196 | 2196 | AACCACAGGAAUUGCUGAG | 7686 | CUCAGCAAUUCCUGUGGUU |
| siRNA 2197 | 2197 | ACCACAGGAAUUGCUGAGC | 7687 | GCUCAGCAAUUCCUGUGGU |
| siRNA 2198 | 2198 | CCACAGGAAUUGCUGAGCU | 7688 | AGCUCAGCAAUUCCUGUGG |
| siRNA 2199 | 2199 | CACAGGAAUUGCUGAGCUU | 7689 | AAGCUCAGCAAUUCCUGUG |
| siRNA 2200 | 2200 | ACAGGAAUUGCUGAGCUUA | 7690 | UAAGCUCAGCAAUUCCUGU |
| siRNA 2201 | 2201 | CAGGAAUUGCUGAGCUUAC | 7691 | GUAAGCUCAGCAAUUCCUG |
| siRNA 2202 | 2202 | AGGAAUUGCUGAGCUUACU | 7692 | AGUAAGCUCAGCAAUUCCU |
| siRNA 2203 | 2203 | GGAAUUGCUGAGCUUACUG | 7693 | CAGUAAGCUCAGCAAUUCC |
| siRNA 2204 | 2204 | GAAUUGCUGAGCUUACUGA | 7694 | UCAGUAAGCUCAGCAAUUC |
| siRNA 2205 | 2205 | AAUUGCUGAGCUUACUGAU | 7695 | AUCAGUAAGCUCAGCAAUU |
| siRNA 2206 | 2206 | AUUGCUGAGCUUACUGAUU | 7696 | AAUCAGUAAGCUCAGCAAU |
| siRNA 2207 | 2207 | UUGCUGAGCUUACUGAUUG | 7697 | CAAUCAGUAAGCUCAGCAA |
| siRNA 2208 | 2208 | UGCUGAGCUUACUGAUUGA | 7698 | UCAAUCAGUAAGCUCAGCA |
| siRNA 2209 | 2209 | GCUGAGCUUACUGAUUGAA | 7699 | UUCAAUCAGUAAGCUCAGC |
| siRNA 2210 | 2210 | CUGAGCUUACUGAUUGAAC | 7700 | GUUCAAUCAGUAAGCUCAG |
| siRNA 2211 | 2211 | UGAGCUUACUGAUUGAACG | 7701 | CGUUCAAUCAGUAAGCUCA |
| siRNA 2212 | 2212 | GAGCUUACUGAUUGAACGG | 7702 | CCGUUCAAUCAGUAAGCUC |
| siRNA 2213 | 2213 | AGCUUACUGAUUGAACGGU | 7703 | ACCGUUCAAUCAGUAAGCU |
| siRNA 2214 | 2214 | GCUUACUGAUUGAACGGUU | 7704 | AACCGUUCAAUCAGUAAGC |
| siRNA 2215 | 2215 | CUUACUGAUUGAACGGUUU | 7705 | AAACCGUUCAAUCAGUAAG |
| siRNA 2216 | 2216 | UUACUGAUUGAACGGUUUG | 7706 | CAAACCGUUCAAUCAGUAA |
| siRNA 2217 | 2217 | UACUGAUUGAACGGUUUGA | 7707 | UCAAACCGUUCAAUCAGUA |
| siRNA 2218 | 2218 | ACUGAUUGAACGGUUUGAA | 7708 | UUCAAACCGUUCAAUCAGU |
| siRNA 2219 | 2219 | CUGAUUGAACGGUUUGAAA | 7709 | UUUCAAACCGUUCAAUCAG |
| siRNA 2220 | 2220 | UGAUUGAACGGUUUGAAAU | 7710 | AUUUCAAACCGUUCAAUCA |
| siRNA 2221 | 2221 | GAUUGAACGGUUUGAAAUU | 7711 | AAUUUCAAACCGUUCAAUC |
| siRNA 2222 | 2222 | AUUGAACGGUUUGAAAUUC | 7712 | GAAUUUCAAACCGUUCAAU |
| siRNA 2223 | 2223 | UUGAACGGUUUGAAAUUCC | 7713 | GGAAUUUCAAACCGUUCAA |
| siRNA 2224 | 2224 | UGAACGGUUUGAAAUUCCA | 7714 | UGGAAUUUCAAACCGUUCA |
| siRNA 2225 | 2225 | GAACGGUUUGAAAUUCCAG | 7715 | CUGGAAUUUCAAACCGUUC |
| siRNA 2226 | 2226 | AACGGUUUGAAAUUCCAGA | 7716 | UCUGGAAUUUCAAACCGUU |
| siRNA 2227 | 2227 | ACGGUUUGAAAUUCCAGAG | 7717 | CUCUGGAAUUUCAAACCGU |
| siRNA 2228 | 2228 | CGGUUUGAAAUUCCAGAGC | 7718 | GCUCUGGAAUUUCAAACCG |
| siRNA 2229 | 2229 | GGUUUGAAAUUCCAGAGCC | 7719 | GGCUCUGGAAUUUCAAACC |
| siRNA 2230 | 2230 | GUUUGAAAUUCCAGAGCCA | 7720 | UGGCUCUGGAAUUUCAAAC |
| siRNA 2231 | 2231 | UUUGAAAUUCCAGAGCCAG | 7721 | CUGGCUCUGGAAUUUCAAA |
| siRNA 2232 | 2232 | UUGAAAUUCCAGAGCCAGA | 7722 | UCUGGCUCUGGAAUUUCAA |
| siRNA 2233 | 2233 | UGAAAUUCCAGAGCCAGAA | 7723 | UUCUGGCUCUGGAAUUUCA |
| siRNA 2234 | 2234 | GAAAUUCCAGAGCCAGAAC | 7724 | GUUCUGGCUCUGGAAUUUC |
| siRNA 2235 | 2235 | AAAUUCCAGAGCCAGAACC | 7725 | GGUUCUGGCUCUGGAAUUU |
| siRNA 2236 | 2236 | AAUUCCAGAGCCAGAACCU | 7726 | AGGUUCUGGCUCUGGAAUU |
| siRNA 2237 | 2237 | AUUCCAGAGCCAGAACCUA | 7727 | UAGGUUCUGGCUCUGGAAU |
| siRNA 2238 | 2238 | UUCCAGAGCCAGAACCUAC | 7728 | GUAGGUUCUGGCUCUGGAA |
| siRNA 2239 | 2239 | UCCAGAGCCAGAACCUACU | 7729 | AGUAGGUUCUGGCUCUGGA |
| siRNA 2240 | 2240 | CCAGAGCCAGAACCUACUG | 7730 | CAGUAGGUUCUGGCUCUGG |
| siRNA 2241 | 2241 | CAGAGCCAGAACCUACUGA | 7731 | UCAGUAGGUUCUGGCUCUG |
| siRNA 2242 | 2242 | AGAGCCAGAACCUACUGAC | 7732 | GUCAGUAGGUUCUGGCUCU |
| siRNA 2243 | 2243 | GAGCCAGAACCUACUGACG | 7733 | CGUCAGUAGGUUCUGGCUC |
| siRNA 2244 | 2244 | AGCCAGAACCUACUGACGC | 7734 | GCGUCAGUAGGUUCUGGCU |
| siRNA 2245 | 2245 | GCCAGAACCUACUGACGCA | 7735 | UGCGUCAGUAGGUUCUGGC |
| siRNA 2246 | 2246 | CCAGAACCUACUGACGCAG | 7736 | CUGCGUCAGUAGGUUCUGG |
| siRNA 2247 | 2247 | CAGAACCUACUGACGCAGA | 7737 | UCUGCGUCAGUAGGUUCUG |
| siRNA 2248 | 2248 | AGAACCUACUGACGCAGAC | 7738 | GUCUGCGUCAGUAGGUUCU |
| siRNA 2249 | 2249 | GAACCUACUGACGCAGACA | 7739 | UGUCUGCGUCAGUAGGUUC |
| siRNA 2250 | 2250 | AACCUACUGACGCAGACAA | 7740 | UUGUCUGCGUCAGUAGGUU |
| siRNA 2251 | 2251 | ACCUACUGACGCAGACAAA | 7741 | UUUGUCUGCGUCAGUAGGU |
| siRNA 2252 | 2252 | CCUACUGACGCAGACAAAU | 7742 | AUUUGUCUGCGUCAGUAGG |
| siRNA 2253 | 2253 | CUACUGACGCAGACAAAUU | 7743 | AAUUUGUCUGCGUCAGUAG |
| siRNA 2254 | 2254 | UACUGACGCAGACAAAUUG | 7744 | CAAUUUGUCUGCGUCAGUA |
| siRNA 2255 | 2255 | ACUGACGCAGACAAAUUGG | 7745 | CCAAUUUGUCUGCGUCAGU |
| siRNA 2256 | 2256 | CUGACGCAGACAAAUUGGC | 7746 | GCCAAUUUGUCUGCGUCAG |
| siRNA 2257 | 2257 | UGACGCAGACAAAUUGGCA | 7747 | UGCCAAUUUGUCUGCGUCA |
| siRNA 2258 | 2258 | GACGCAGACAAAUUGGCAA | 7748 | UUGCCAAUUUGUCUGCGUC |
| siRNA 2259 | 2259 | ACGCAGACAAAUUGGCAAU | 7749 | AUUGCCAAUUUGUCUGCGU |
| siRNA 2260 | 2260 | CGCAGACAAAUUGGCAAUA | 7750 | UAUUGCCAAUUUGUCUGCG |
| siRNA 2261 | 2261 | GCAGACAAAUUGGCAAUAG | 7751 | CUAUUGCCAAUUUGUCUGC |
| siRNA 2262 | 2262 | CAGACAAAUUGGCAAUAGA | 7752 | UCUAUUGCCAAUUUGUCUG |
| siRNA 2263 | 2263 | AGACAAAUUGGCAAUAGAG | 7753 | CUCUAUUGCCAAUUUGUCU |
| siRNA 2264 | 2264 | GACAAAUUGGCAAUAGAGA | 7754 | UCUCUAUUGCCAAUUUGUC |
| siRNA 2265 | 2265 | ACAAAUUGGCAAUAGAGAA | 7755 | UUCUCUAUUGCCAAUUUGU |
| siRNA 2266 | 2266 | CAAAUUGGCAAUAGAGAAA | 7756 | UUUCUCUAUUGCCAAUUUG |
| siRNA 2267 | 2267 | AAAUUGGCAAUAGAGAAAG | 7757 | CUUUCUCUAUUGCCAAUUU |
| siRNA 2268 | 2268 | AAUUGGCAAUAGAGAAAGG | 7758 | CCUUUCUCUAUUGCCAAUU |
| siRNA 2269 | 2269 | AUUGGCAAUAGAGAAAGGC | 7759 | GCCUUUCUCUAUUGCCAAU |
| siRNA 2270 | 2270 | UUGGCAAUAGAGAAAGGCG | 7760 | CGCCUUUCUCUAUUGCCAA |
| siRNA 2271 | 2271 | UGGCAAUAGAGAAAGGCGA | 7761 | UCGCCUUUCUCUAUUGCCA |
| siRNA 2272 | 2272 | GGCAAUAGAGAAAGGCGAG | 7762 | CUCGCCUUUCUCUAUUGCC |
| siRNA 2273 | 2273 | GCAAUAGAGAAAGGCGAGC | 7763 | GCUCGCCUUUCUCUAUUGC |
| siRNA 2274 | 2274 | CAAUAGAGAAAGGCGAGCA | 7764 | UGCUCGCCUUUCUCUAUUG |
| siRNA 2275 | 2275 | AAUAGAGAAAGGCGAGCAG | 7765 | CUGCUCGCCUUUCUCUAUU |
| siRNA 2276 | 2276 | AUAGAGAAAGGCGAGCAGC | 7766 | GCUGCUCGCCUUUCUCUAU |
| siRNA 2277 | 2277 | UAGAGAAAGGCGAGCAGCC | 7767 | GGCUGCUCGCCUUUCUCUA |
| siRNA 2278 | 2278 | AGAGAAAGGCGAGCAGCCA | 7768 | UGGCUGCUCGCCUUUCUCU |
| siRNA 2279 | 2279 | GAGAAAGGCGAGCAGCCAA | 7769 | UUGGCUGCUCGCCUUUCUC |
| siRNA 2280 | 2280 | AGAAAGGCGAGCAGCCAAU | 7770 | AUUGGCUGCUCGCCUUUCU |
| siRNA 2281 | 2281 | GAAAGGCGAGCAGCCAAUC | 7771 | GAUUGGCUGCUCGCCUUUC |
| siRNA 2282 | 2282 | AAAGGCGAGCAGCCAAUCA | 7772 | UGAUUGGCUGCUCGCCUUU |
| siRNA 2283 | 2283 | AAGGCGAGCAGCCAAUCAG | 7773 | CUGAUUGGCUGCUCGCCUU |
| siRNA 2284 | 2284 | AGGCGAGCAGCCAAUCAGU | 7774 | ACUGAUUGGCUGCUCGCCU |
| siRNA 2285 | 2285 | GGCGAGCAGCCAAUCAGUG | 7775 | CACUGAUUGGCUGCUCGCC |
| siRNA 2286 | 2286 | GCGAGCAGCCAAUCAGUGC | 7776 | GCACUGAUUGGCUGCUCGC |
| siRNA 2287 | 2287 | CGAGCAGCCAAUCAGUGCA | 7777 | UGCACUGAUUGGCUGCUCG |
| siRNA 2288 | 2288 | GAGCAGCCAAUCAGUGCAG | 7778 | CUGCACUGAUUGGCUGCUC |
| siRNA 2289 | 2289 | AGCAGCCAAUCAGUGCAGA | 7779 | UCUGCACUGAUUGGCUGCU |
| siRNA 2290 | 2290 | GCAGCCAAUCAGUGCAGAC | 7780 | GUCUGCACUGAUUGGCUGC |
| siRNA 2291 | 2291 | CAGCCAAUCAGUGCAGACC | 7781 | GGUCUGCACUGAUUGGCUG |
| siRNA 2292 | 2292 | AGCCAAUCAGUGCAGACCU | 7782 | AGGUCUGCACUGAUUGGCU |
| siRNA 2293 | 2293 | GCCAAUCAGUGCAGACCUU | 7783 | AAGGUCUGCACUGAUUGGC |
| siRNA 2294 | 2294 | CCAAUCAGUGCAGACCUUA | 7784 | UAAGGUCUGCACUGAUUGG |
| siRNA 2295 | 2295 | CAAUCAGUGCAGACCUUAA | 7785 | UUAAGGUCUGCACUGAUUG |
| siRNA 2296 | 2296 | AAUCAGUGCAGACCUUAAA | 7786 | UUUAAGGUCUGCACUGAUU |
| siRNA 2297 | 2297 | AUCAGUGCAGACCUUAAAA | 7787 | UUUUAAGGUCUGCACUGAU |
| siRNA 2298 | 2298 | UCAGUGCAGACCUUAAAAG | 7788 | CUUUUAAGGUCUGCACUGA |
| siRNA 2299 | 2299 | CAGUGCAGACCUUAAAAGA | 7789 | UCUUUUAAGGUCUGCACUG |
| siRNA 2300 | 2300 | AGUGCAGACCUUAAAAGAU | 7790 | AUCUUUUAAGGUCUGCACU |
| siRNA 2301 | 2301 | GUGCAGACCUUAAAAGAUU | 7791 | AAUCUUUUAAGGUCUGCAC |
| siRNA 2302 | 2302 | UGCAGACCUUAAAAGAUUU | 7792 | AAAUCUUUUAAGGUCUGCA |
| siRNA 2303 | 2303 | GCAGACCUUAAAAGAUUUC | 7793 | GAAAUCUUUUAAGGUCUGC |
| siRNA 2304 | 2304 | CAGACCUUAAAAGAUUUCG | 7794 | CGAAAUCUUUUAAGGUCUG |
| siRNA 2305 | 2305 | AGACCUUAAAAGAUUUCGC | 7795 | GCGAAAUCUUUUAAGGUCU |
| siRNA 2306 | 2306 | GACCUUAAAAGAUUUCGCA | 7796 | UGCGAAAUCUUUUAAGGUC |
| siRNA 2307 | 2307 | ACCUUAAAAGAUUUCGCAA | 7797 | UUGCGAAAUCUUUUAAGGU |
| siRNA 2308 | 2308 | CCUUAAAAGAUUUCGCAAG | 7798 | CUUGCGAAAUCUUUUAAGG |
| siRNA 2309 | 2309 | CUUAAAAGAUUUCGCAAGG | 7799 | CCUUGCGAAAUCUUUUAAG |
| siRNA 2310 | 2310 | UUAAAAGAUUUCGCAAGGA | 7800 | UCCUUGCGAAAUCUUUUAA |
| siRNA 2311 | 2311 | UAAAAGAUUUCGCAAGGAA | 7801 | UUCCUUGCGAAAUCUUUUA |
| siRNA 2312 | 2312 | AAAAGAUUUCGCAAGGAAU | 7802 | AUUCCUUGCGAAAUCUUUU |
| siRNA 2313 | 2313 | AAAGAUUUCGCAAGGAAUA | 7803 | UAUUCCUUGCGAAAUCUUU |
| siRNA 2314 | 2314 | AAGAUUUCGCAAGGAAUAU | 7804 | AUAUUCCUUGCGAAAUCUU |
| siRNA 2315 | 2315 | AGAUUUCGCAAGGAAUAUG | 7805 | CAUAUUCCUUGCGAAAUCU |
| siRNA 2316 | 2316 | GAUUUCGCAAGGAAUAUGU | 7806 | ACAUAUUCCUUGCGAAAUC |
| siRNA 2317 | 2317 | AUUUCGCAAGGAAUAUGUC | 7807 | GACAUAUUCCUUGCGAAAU |
| siRNA 2318 | 2318 | UUUCGCAAGGAAUAUGUCC | 7808 | GGACAUAUUCCUUGCGAAA |
| siRNA 2319 | 2319 | UUCGCAAGGAAUAUGUCCA | 7809 | UGGACAUAUUCCUUGCGAA |
| siRNA 2320 | 2320 | UCGCAAGGAAUAUGUCCAA | 7810 | UUGGACAUAUUCCUUGCGA |
| siRNA 2321 | 2321 | CGCAAGGAAUAUGUCCAAC | 7811 | GUUGGACAUAUUCCUUGCG |
| siRNA 2322 | 2322 | GCAAGGAAUAUGUCCAACC | 7812 | GGUUGGACAUAUUCCUUGC |
| siRNA 2323 | 2323 | CAAGGAAUAUGUCCAACCA | 7813 | UGGUUGGACAUAUUCCUUG |
| siRNA 2324 | 2324 | AAGGAAUAUGUCCAACCAG | 7814 | CUGGUUGGACAUAUUCCUU |
| siRNA 2325 | 2325 | AGGAAUAUGUCCAACCAGU | 7815 | ACUGGUUGGACAUAUUCCU |
| siRNA 2326 | 2326 | GGAAUAUGUCCAACCAGUA | 7816 | UACUGGUUGGACAUAUUCC |
| siRNA 2327 | 2327 | GAAUAUGUCCAACCAGUAC | 7817 | GUACUGGUUGGACAUAUUC |
| siRNA 2328 | 2328 | AAUAUGUCCAACCAGUACA | 7818 | UGUACUGGUUGGACAUAUU |
| siRNA 2329 | 2329 | AUAUGUCCAACCAGUACAA | 7819 | UUGUACUGGUUGGACAUAU |
| siRNA 2330 | 2330 | UAUGUCCAACCAGUACAAC | 7820 | GUUGUACUGGUUGGACAUA |
| siRNA 2331 | 2331 | AUGUCCAACCAGUACAACU | 7821 | AGUUGUACUGGUUGGACAU |
| siRNA 2332 | 2332 | UGUCCAACCAGUACAACUU | 7822 | AAGUUGUACUGGUUGGACA |
| siRNA 2333 | 2333 | GUCCAACCAGUACAACUUA | 7823 | UAAGUUGUACUGGUUGGAC |
| siRNA 2334 | 2334 | UCCAACCAGUACAACUUAG | 7824 | CUAAGUUGUACUGGUUGGA |
| siRNA 2335 | 2335 | CCAACCAGUACAACUUAGG | 7825 | CCUAAGUUGUACUGGUUGG |
| siRNA 2336 | 2336 | CAACCAGUACAACUUAGGA | 7826 | UCCUAAGUUGUACUGGUUG |
| siRNA 2337 | 2337 | AACCAGUACAACUUAGGAU | 7827 | AUCCUAAGUUGUACUGGUU |
| siRNA 2338 | 2338 | ACCAGUACAACUUAGGAUC | 7828 | GAUCCUAAGUUGUACUGGU |
| siRNA 2339 | 2339 | CCAGUACAACUUAGGAUCU | 7829 | AGAUCCUAAGUUGUACUGG |
| siRNA 2340 | 2340 | CAGUACAACUUAGGAUCUU | 7830 | AAGAUCCUAAGUUGUACUG |
| siRNA 2341 | 2341 | AGUACAACUUAGGAUCUUA | 7831 | UAAGAUCCUAAGUUGUACU |
| siRNA 2342 | 2342 | GUACAACUUAGGAUCUUAA | 7832 | UUAAGAUCCUAAGUUGUAC |
| siRNA 2343 | 2343 | UACAACUUAGGAUCUUAAA | 7833 | UUUAAGAUCCUAAGUUGUA |
| siRNA 2344 | 2344 | ACAACUUAGGAUCUUAAAU | 7834 | AUUUAAGAUCCUAAGUUGU |
| siRNA 2345 | 2345 | CAACUUAGGAUCUUAAAUG | 7835 | CAUUUAAGAUCCUAAGUUG |
| siRNA 2346 | 2346 | AACUUAGGAUCUUAAAUGU | 7836 | ACAUUUAAGAUCCUAAGUU |
| siRNA 2347 | 2347 | ACUUAGGAUCUUAAAUGUA | 7837 | UACAUUUAAGAUCCUAAGU |
| siRNA 2348 | 2348 | CUUAGGAUCUUAAAUGUAU | 7838 | AUACAUUUAAGAUCCUAAG |
| siRNA 2349 | 2349 | UUAGGAUCUUAAAUGUAUU | 7839 | AAUACAUUUAAGAUCCUAA |
| siRNA 2350 | 2350 | UAGGAUCUUAAAUGUAUUU | 7840 | AAAUACAUUUAAGAUCCUA |
| siRNA 2351 | 2351 | AGGAUCUUAAAUGUAUUUC | 7841 | GAAAUACAUUUAAGAUCCU |
| siRNA 2352 | 2352 | GGAUCUUAAAUGUAUUUCG | 7842 | CGAAAUACAUUUAAGAUCC |
| siRNA 2353 | 2353 | GAUCUUAAAUGUAUUUCGG | 7843 | CCGAAAUACAUUUAAGAUC |
| siRNA 2354 | 2354 | AUCUUAAAUGUAUUUCGGC | 7844 | GCCGAAAUACAUUUAAGAU |
| siRNA 2355 | 2355 | UCUUAAAUGUAUUUCGGCA | 7845 | UGCCGAAAUACAUUUAAGA |
| siRNA 2356 | 2356 | CUUAAAUGUAUUUCGGCAU | 7846 | AUGCCGAAAUACAUUUAAG |
| siRNA 2357 | 2357 | UUAAAUGUAUUUCGGCAUU | 7847 | AAUGCCGAAAUACAUUUAA |
| siRNA 2358 | 2358 | UAAAUGUAUUUCGGCAUUG | 7848 | CAAUGCCGAAAUACAUUUA |
| siRNA 2359 | 2359 | AAAUGUAUUUCGGCAUUGG | 7849 | CCAAUGCCGAAAUACAUUU |
| siRNA 2360 | 2360 | AAUGUAUUUCGGCAUUGGG | 7850 | CCCAAUGCCGAAAUACAUU |
| siRNA 2361 | 2361 | AUGUAUUUCGGCAUUGGGU | 7851 | ACCCAAUGCCGAAAUACAU |
| siRNA 2362 | 2362 | UGUAUUUCGGCAUUGGGUU | 7852 | AACCCAAUGCCGAAAUACA |
| siRNA 2363 | 2363 | GUAUUUCGGCAUUGGGUUG | 7853 | CAACCCAAUGCCGAAAUAC |
| siRNA 2364 | 2364 | UAUUUCGGCAUUGGGUUGA | 7854 | UCAACCCAAUGCCGAAAUA |
| siRNA 2365 | 2365 | AUUUCGGCAUUGGGUUGAA | 7855 | UUCAACCCAAUGCCGAAAU |
| siRNA 2366 | 2366 | UUUCGGCAUUGGGUUGAAC | 7856 | GUUCAACCCAAUGCCGAAA |
| siRNA 2367 | 2367 | UUCGGCAUUGGGUUGAACA | 7857 | UGUUCAACCCAAUGCCGAA |
| siRNA 2368 | 2368 | UCGGCAUUGGGUUGAACAU | 7858 | AUGUUCAACCCAAUGCCGA |
| siRNA 2369 | 2369 | CGGCAUUGGGUUGAACAUC | 7859 | GAUGUUCAACCCAAUGCCG |
| siRNA 2370 | 2370 | GGCAUUGGGUUGAACAUCA | 7860 | UGAUGUUCAACCCAAUGCC |
| siRNA 2371 | 2371 | GCAUUGGGUUGAACAUCAU | 7861 | AUGAUGUUCAACCCAAUGC |
| siRNA 2372 | 2372 | CAUUGGGUUGAACAUCAUU | 7862 | AAUGAUGUUCAACCCAAUG |
| siRNA 2373 | 2373 | AUUGGGUUGAACAUCAUUU | 7863 | AAAUGAUGUUCAACCCAAU |
| siRNA 2374 | 2374 | UUGGGUUGAACAUCAUUUU | 7864 | AAAAUGAUGUUCAACCCAA |
| siRNA 2375 | 2375 | UGGGUUGAACAUCAUUUUU | 7865 | AAAAAUGAUGUUCAACCCA |
| siRNA 2376 | 2376 | GGGUUGAACAUCAUUUUUA | 7866 | UAAAAAUGAUGUUCAACCC |
| siRNA 2377 | 2377 | GGUUGAACAUCAUUUUUAU | 7867 | AUAAAAAUGAUGUUCAACC |
| siRNA 2378 | 2378 | GUUGAACAUCAUUUUUAUG | 7868 | CAUAAAAAUGAUGUUCAAC |
| siRNA 2379 | 2379 | UUGAACAUCAUUUUUAUGA | 7869 | UCAUAAAAAUGAUGUUCAA |
| siRNA 2380 | 2380 | UGAACAUCAUUUUUAUGAC | 7870 | GUCAUAAAAAUGAUGUUCA |
| siRNA 2381 | 2381 | GAACAUCAUUUUUAUGACU | 7871 | AGUCAUAAAAAUGAUGUUC |
| siRNA 2382 | 2382 | AACAUCAUUUUUAUGACUU | 7872 | AAGUCAUAAAAAUGAUGUU |
| siRNA 2383 | 2383 | ACAUCAUUUUUAUGACUUU | 7873 | AAAGUCAUAAAAAUGAUGU |
| siRNA 2384 | 2384 | CAUCAUUUUUAUGACUUUG | 7874 | CAAAGUCAUAAAAAUGAUG |
| siRNA 2385 | 2385 | AUCAUUUUUAUGACUUUGA | 7875 | UCAAAGUCAUAAAAAUGAU |
| siRNA 2386 | 2386 | UCAUUUUUAUGACUUUGAA | 7876 | UUCAAAGUCAUAAAAAUGA |
| siRNA 2387 | 2387 | CAUUUUUAUGACUUUGAAA | 7877 | UUUCAAAGUCAUAAAAAUG |
| siRNA 2388 | 2388 | AUUUUUAUGACUUUGAAAG | 7878 | CUUUCAAAGUCAUAAAAAU |
| siRNA 2389 | 2389 | UUUUUAUGACUUUGAAAGA | 7879 | UCUUUCAAAGUCAUAAAAA |
| siRNA 2390 | 2390 | UUUUAUGACUUUGAAAGAG | 7880 | CUCUUUCAAAGUCAUAAAA |
| siRNA 2391 | 2391 | UUUAUGACUUUGAAAGAGA | 7881 | UCUCUUUCAAAGUCAUAAA |
| siRNA 2392 | 2392 | UUAUGACUUUGAAAGAGAC | 7882 | GUCUCUUUCAAAGUCAUAA |
| siRNA 2393 | 2393 | UAUGACUUUGAAAGAGACU | 7883 | AGUCUCUUUCAAAGUCAUA |
| siRNA 2394 | 2394 | AUGACUUUGAAAGAGACUU | 7884 | AAGUCUCUUUCAAAGUCAU |
| siRNA 2395 | 2395 | UGACUUUGAAAGAGACUUG | 7885 | CAAGUCUCUUUCAAAGUCA |
| siRNA 2396 | 2396 | GACUUUGAAAGAGACUUGG | 7886 | CCAAGUCUCUUUCAAAGUC |
| siRNA 2397 | 2397 | ACUUUGAAAGAGACUUGGA | 7887 | UCCAAGUCUCUUUCAAAGU |
| siRNA 2398 | 2398 | CUUUGAAAGAGACUUGGAA | 7888 | UUCCAAGUCUCUUUCAAAG |
| siRNA 2399 | 2399 | UUUGAAAGAGACUUGGAAU | 7889 | AUUCCAAGUCUCUUUCAAA |
| siRNA 2400 | 2400 | UUGAAAGAGACUUGGAAUU | 7890 | AAUUCCAAGUCUCUUUCAA |
| siRNA 2401 | 2401 | UGAAAGAGACUUGGAAUUG | 7891 | CAAUUCCAAGUCUCUUUCA |
| siRNA 2402 | 2402 | GAAAGAGACUUGGAAUUGC | 7892 | GCAAUUCCAAGUCUCUUUC |
| siRNA 2403 | 2403 | AAAGAGACUUGGAAUUGCU | 7893 | AGCAAUUCCAAGUCUCUUU |
| siRNA 2404 | 2404 | AAGAGACUUGGAAUUGCUU | 7894 | AAGCAAUUCCAAGUCUCUU |
| siRNA 2405 | 2405 | AGAGACUUGGAAUUGCUUG | 7895 | CAAGCAAUUCCAAGUCUCU |
| siRNA 2406 | 2406 | GAGACUUGGAAUUGCUUGA | 7896 | UCAAGCAAUUCCAAGUCUC |
| siRNA 2407 | 2407 | AGACUUGGAAUUGCUUGAA | 7897 | UUCAAGCAAUUCCAAGUCU |
| siRNA 2408 | 2408 | GACUUGGAAUUGCUUGAAA | 7898 | UUUCAAGCAAUUCCAAGUC |
| siRNA 2409 | 2409 | ACUUGGAAUUGCUUGAAAG | 7899 | CUUUCAAGCAAUUCCAAGU |
| siRNA 2410 | 2410 | CUUGGAAUUGCUUGAAAGA | 7900 | UCUUUCAAGCAAUUCCAAG |
| siRNA 2411 | 2411 | UUGGAAUUGCUUGAAAGAC | 7901 | GUCUUUCAAGCAAUUCCAA |
| siRNA 2412 | 2412 | UGGAAUUGCUUGAAAGACU | 7902 | AGUCUUUCAAGCAAUUCCA |
| siRNA 2413 | 2413 | GGAAUUGCUUGAAAGACUA | 7903 | UAGUCUUUCAAGCAAUUCC |
| siRNA 2414 | 2414 | GAAUUGCUUGAAAGACUAG | 7904 | CUAGUCUUUCAAGCAAUUC |
| siRNA 2415 | 2415 | AAUUGCUUGAAAGACUAGA | 7905 | UCUAGUCUUUCAAGCAAUU |
| siRNA 2416 | 2416 | AUUGCUUGAAAGACUAGAA | 7906 | UUCUAGUCUUUCAAGCAAU |
| siRNA 2417 | 2417 | UUGCUUGAAAGACUAGAAU | 7907 | AUUCUAGUCUUUCAAGCAA |
| siRNA 2418 | 2418 | UGCUUGAAAGACUAGAAUC | 7908 | GAUUCUAGUCUUUCAAGCA |
| siRNA 2419 | 2419 | GCUUGAAAGACUAGAAUCC | 7909 | GGAUUCUAGUCUUUCAAGC |
| siRNA 2420 | 2420 | CUUGAAAGACUAGAAUCCU | 7910 | AGGAUUCUAGUCUUUCAAG |
| siRNA 2421 | 2421 | UUGAAAGACUAGAAUCCUU | 7911 | AAGGAUUCUAGUCUUUCAA |
| siRNA 2422 | 2422 | UGAAAGACUAGAAUCCUUC | 7912 | GAAGGAUUCUAGUCUUUCA |
| siRNA 2423 | 2423 | GAAAGACUAGAAUCCUUCA | 7913 | UGAAGGAUUCUAGUCUUUC |
| siRNA 2424 | 2424 | AAAGACUAGAAUCCUUCAU | 7914 | AUGAAGGAUUCUAGUCUUU |
| siRNA 2425 | 2425 | AAGACUAGAAUCCUUCAUU | 7915 | AAUGAAGGAUUCUAGUCUU |
| siRNA 2426 | 2426 | AGACUAGAAUCCUUCAUUU | 7916 | AAAUGAAGGAUUCUAGUCU |
| siRNA 2427 | 2427 | GACUAGAAUCCUUCAUUUC | 7917 | GAAAUGAAGGAUUCUAGUC |
| siRNA 2428 | 2428 | ACUAGAAUCCUUCAUUUCA | 7918 | UGAAAUGAAGGAUUCUAGU |
| siRNA 2429 | 2429 | CUAGAAUCCUUCAUUUCAA | 7919 | UUGAAAUGAAGGAUUCUAG |
| siRNA 2430 | 2430 | UAGAAUCCUUCAUUUCAAG | 7920 | CUUGAAAUGAAGGAUUCUA |
| siRNA 2431 | 2431 | AGAAUCCUUCAUUUCAAGU | 7921 | ACUUGAAAUGAAGGAUUCU |
| siRNA 2432 | 2432 | GAAUCCUUCAUUUCAAGUG | 7922 | CACUUGAAAUGAAGGAUUC |
| siRNA 2433 | 2433 | AAUCCUUCAUUUCAAGUGU | 7923 | ACACUUGAAAUGAAGGAUU |
| siRNA 2434 | 2434 | AUCCUUCAUUUCAAGUGUA | 7924 | UACACUUGAAAUGAAGGAU |
| siRNA 2435 | 2435 | UCCUUCAUUUCAAGUGUAA | 7925 | UUACACUUGAAAUGAAGGA |
| siRNA 2436 | 2436 | CCUUCAUUUCAAGUGUAAG | 7926 | CUUACACUUGAAAUGAAGG |
| siRNA 2437 | 2437 | CUUCAUUUCAAGUGUAAGA | 7927 | UCUUACACUUGAAAUGAAG |
| siRNA 2438 | 2438 | UUCAUUUCAAGUGUAAGAG | 7928 | CUCUUACACUUGAAAUGAA |
| siRNA 2439 | 2439 | UCAUUUCAAGUGUAAGAGG | 7929 | CCUCUUACACUUGAAAUGA |
| siRNA 2440 | 2440 | CAUUUCAAGUGUAAGAGGG | 7930 | CCCUCUUACACUUGAAAUG |
| siRNA 2441 | 2441 | AUUUCAAGUGUAAGAGGGA | 7931 | UCCCUCUUACACUUGAAAU |
| siRNA 2442 | 2442 | UUUCAAGUGUAAGAGGGAA | 7932 | UUCCCUCUUACACUUGAAA |
| siRNA 2443 | 2443 | UUCAAGUGUAAGAGGGAAA | 7933 | UUUCCCUCUUACACUUGAA |
| siRNA 2444 | 2444 | UCAAGUGUAAGAGGGAAAG | 7934 | CUUUCCCUCUUACACUUGA |
| siRNA 2445 | 2445 | CAAGUGUAAGAGGGAAAGC | 7935 | GCUUUCCCUCUUACACUUG |
| siRNA 2446 | 2446 | AAGUGUAAGAGGGAAAGCU | 7936 | AGCUUUCCCUCUUACACUU |
| siRNA 2447 | 2447 | AGUGUAAGAGGGAAAGCUA | 7937 | UAGCUUUCCCUCUUACACU |
| siRNA 2448 | 2448 | GUGUAAGAGGGAAAGCUAU | 7938 | AUAGCUUUCCCUCUUACAC |
| siRNA 2449 | 2449 | UGUAAGAGGGAAAGCUAUG | 7939 | CAUAGCUUUCCCUCUUACA |
| siRNA 2450 | 2450 | GUAAGAGGGAAAGCUAUGA | 7940 | UCAUAGCUUUCCCUCUUAC |
| siRNA 2451 | 2451 | UAAGAGGGAAAGCUAUGAA | 7941 | UUCAUAGCUUUCCCUCUUA |
| siRNA 2452 | 2452 | AAGAGGGAAAGCUAUGAAA | 7942 | UUUCAUAGCUUUCCCUCUU |
| siRNA 2453 | 2453 | AGAGGGAAAGCUAUGAAAA | 7943 | UUUUCAUAGCUUUCCCUCU |
| siRNA 2454 | 2454 | GAGGGAAAGCUAUGAAAAA | 7944 | UUUUUCAUAGCUUUCCCUC |
| siRNA 2455 | 2455 | AGGGAAAGCUAUGAAAAAA | 7945 | UUUUUUCAUAGCUUUCCCU |
| siRNA 2456 | 2456 | GGGAAAGCUAUGAAAAAAU | 7946 | AUUUUUUCAUAGCUUUCCC |
| siRNA 2457 | 2457 | GGAAAGCUAUGAAAAAAUG | 7947 | CAUUUUUUCAUAGCUUUCC |
| siRNA 2458 | 2458 | GAAAGCUAUGAAAAAAUGG | 7948 | CCAUUUUUUCAUAGCUUUC |
| siRNA 2459 | 2459 | AAAGCUAUGAAAAAAUGGG | 7949 | CCCAUUUUUUCAUAGCUUU |
| siRNA 2460 | 2460 | AAGCUAUGAAAAAAUGGGU | 7950 | ACCCAUUUUUUCAUAGCUU |
| siRNA 2461 | 2461 | AGCUAUGAAAAAAUGGGUA | 7951 | UACCCAUUUUUUCAUAGCU |
| siRNA 2462 | 2462 | GCUAUGAAAAAAUGGGUAG | 7952 | CUACCCAUUUUUUCAUAGC |
| siRNA 2463 | 2463 | CUAUGAAAAAAUGGGUAGA | 7953 | UCUACCCAUUUUUUCAUAG |
| siRNA 2464 | 2464 | UAUGAAAAAAUGGGUAGAG | 7954 | CUCUACCCAUUUUUUCAUA |
| siRNA 2465 | 2465 | AUGAAAAAAUGGGUAGAGU | 7955 | ACUCUACCCAUUUUUUCAU |
| siRNA 2466 | 2466 | UGAAAAAAUGGGUAGAGUC | 7956 | GACUCUACCCAUUUUUUCA |
| siRNA 2467 | 2467 | GAAAAAAUGGGUAGAGUCA | 7957 | UGACUCUACCCAUUUUUUC |
| siRNA 2468 | 2468 | AAAAAAUGGGUAGAGUCAA | 7958 | UUGACUCUACCCAUUUUUU |
| siRNA 2469 | 2469 | AAAAAUGGGUAGAGUCAAU | 7959 | AUUGACUCUACCCAUUUUU |
| siRNA 2470 | 2470 | AAAAUGGGUAGAGUCAAUU | 7960 | AAUUGACUCUACCCAUUUU |
| siRNA 2471 | 2471 | AAAUGGGUAGAGUCAAUUG | 7961 | CAAUUGACUCUACCCAUUU |
| siRNA 2472 | 2472 | AAUGGGUAGAGUCAAUUGC | 7962 | GCAAUUGACUCUACCCAUU |
| siRNA 2473 | 2473 | AUGGGUAGAGUCAAUUGCU | 7963 | AGCAAUUGACUCUACCCAU |
| siRNA 2474 | 2474 | UGGGUAGAGUCAAUUGCUA | 7964 | UAGCAAUUGACUCUACCCA |
| siRNA 2475 | 2475 | GGGUAGAGUCAAUUGCUAA | 7965 | UUAGCAAUUGACUCUACCC |
| siRNA 2476 | 2476 | GGUAGAGUCAAUUGCUAAG | 7966 | CUUAGCAAUUGACUCUACC |
| siRNA 2477 | 2477 | GUAGAGUCAAUUGCUAAGA | 7967 | UCUUAGCAAUUGACUCUAC |
| siRNA 2478 | 2478 | UAGAGUCAAUUGCUAAGAU | 7968 | AUCUUAGCAAUUGACUCUA |
| siRNA 2479 | 2479 | AGAGUCAAUUGCUAAGAUC | 7969 | GAUCUUAGCAAUUGACUCU |
| siRNA 2480 | 2480 | GAGUCAAUUGCUAAGAUCA | 7970 | UGAUCUUAGCAAUUGACUC |
| siRNA 2481 | 2481 | AGUCAAUUGCUAAGAUCAU | 7971 | AUGAUCUUAGCAAUUGACU |
| siRNA 2482 | 2482 | GUCAAUUGCUAAGAUCAUC | 7972 | GAUGAUCUUAGCAAUUGAC |
| siRNA 2483 | 2483 | UCAAUUGCUAAGAUCAUCA | 7973 | UGAUGAUCUUAGCAAUUGA |
| siRNA 2484 | 2484 | CAAUUGCUAAGAUCAUCAG | 7974 | CUGAUGAUCUUAGCAAUUG |
| siRNA 2485 | 2485 | AAUUGCUAAGAUCAUCAGG | 7975 | CCUGAUGAUCUUAGCAAUU |
| siRNA 2486 | 2486 | AUUGCUAAGAUCAUCAGGA | 7976 | UCCUGAUGAUCUUAGCAAU |
| siRNA 2487 | 2487 | UUGCUAAGAUCAUCAGGAG | 7977 | CUCCUGAUGAUCUUAGCAA |
| siRNA 2488 | 2488 | UGCUAAGAUCAUCAGGAGG | 7978 | CCUCCUGAUGAUCUUAGCA |
| siRNA 2489 | 2489 | GCUAAGAUCAUCAGGAGGA | 7979 | UCCUCCUGAUGAUCUUAGC |
| siRNA 2490 | 2490 | CUAAGAUCAUCAGGAGGAA | 7980 | UUCCUCCUGAUGAUCUUAG |
| siRNA 2491 | 2491 | UAAGAUCAUCAGGAGGAAG | 7981 | CUUCCUCCUGAUGAUCUUA |
| siRNA 2492 | 2492 | AAGAUCAUCAGGAGGAAGA | 7982 | UCUUCCUCCUGAUGAUCUU |
| siRNA 2493 | 2493 | AGAUCAUCAGGAGGAAGAA | 7983 | UUCUUCCUCCUGAUGAUCU |
| siRNA 2494 | 2494 | GAUCAUCAGGAGGAAGAAG | 7984 | CUUCUUCCUCCUGAUGAUC |
| siRNA 2495 | 2495 | AUCAUCAGGAGGAAGAAGC | 7985 | GCUUCUUCCUCCUGAUGAU |
| siRNA 2496 | 2496 | UCAUCAGGAGGAAGAAGCA | 7986 | UGCUUCUUCCUCCUGAUGA |
| siRNA 2497 | 2497 | CAUCAGGAGGAAGAAGCAA | 7987 | UUGCUUCUUCCUCCUGAUG |
| siRNA 2498 | 2498 | AUCAGGAGGAAGAAGCAAG | 7988 | CUUGCUUCUUCCUCCUGAU |
| siRNA 2499 | 2499 | UCAGGAGGAAGAAGCAAGC | 7989 | GCUUGCUUCUUCCUCCUGA |
| siRNA 2500 | 2500 | CAGGAGGAAGAAGCAAGCU | 7990 | AGCUUGCUUCUUCCUCCUG |
| siRNA 2501 | 2501 | AGGAGGAAGAAGCAAGCUC | 7991 | GAGCUUGCUUCUUCCUCCU |
| siRNA 2502 | 2502 | GGAGGAAGAAGCAAGCUCA | 7992 | UGAGCUUGCUUCUUCCUCC |
| siRNA 2503 | 2503 | GAGGAAGAAGCAAGCUCAG | 7993 | CUGAGCUUGCUUCUUCCUC |
| siRNA 2504 | 2504 | AGGAAGAAGCAAGCUCAGG | 7994 | CCUGAGCUUGCUUCUUCCU |
| siRNA 2505 | 2505 | GGAAGAAGCAAGCUCAGGC | 7995 | GCCUGAGCUUGCUUCUUCC |
| siRNA 2506 | 2506 | GAAGAAGCAAGCUCAGGCA | 7996 | UGCCUGAGCUUGCUUCUUC |
| siRNA 2507 | 2507 | AAGAAGCAAGCUCAGGCAA | 7997 | UUGCCUGAGCUUGCUUCUU |
| siRNA 2508 | 2508 | AGAAGCAAGCUCAGGCAAA | 7998 | UUUGCCUGAGCUUGCUUCU |
| siRNA 2509 | 2509 | GAAGCAAGCUCAGGCAAAC | 7999 | GUUUGCCUGAGCUUGCUUC |
| siRNA 2510 | 2510 | AAGCAAGCUCAGGCAAACG | 8000 | CGUUUGCCUGAGCUUGCUU |
| siRNA 2511 | 2511 | AGCAAGCUCAGGCAAACGG | 8001 | CCGUUUGCCUGAGCUUGCU |
| siRNA 2512 | 2512 | GCAAGCUCAGGCAAACGGA | 8002 | UCCGUUUGCCUGAGCUUGC |
| siRNA 2513 | 2513 | CAAGCUCAGGCAAACGGAG | 8003 | CUCCGUUUGCCUGAGCUUG |
| siRNA 2514 | 2514 | AAGCUCAGGCAAACGGAGU | 8004 | ACUCCGUUUGCCUGAGCUU |
| siRNA 2515 | 2515 | AGCUCAGGCAAACGGAGUA | 8005 | UACUCCGUUUGCCUGAGCU |
| siRNA 2516 | 2516 | GCUCAGGCAAACGGAGUAA | 8006 | UUACUCCGUUUGCCUGAGC |
| siRNA 2517 | 2517 | CUCAGGCAAACGGAGUAAG | 8007 | CUUACUCCGUUUGCCUGAG |
| siRNA 2518 | 2518 | UCAGGCAAACGGAGUAAGC | 8008 | GCUUACUCCGUUUGCCUGA |
| siRNA 2519 | 2519 | CAGGCAAACGGAGUAAGCC | 8009 | GGCUUACUCCGUUUGCCUG |
| siRNA 2520 | 2520 | AGGCAAACGGAGUAAGCCA | 8010 | UGGCUUACUCCGUUUGCCU |
| siRNA 2521 | 2521 | GGCAAACGGAGUAAGCCAU | 8011 | AUGGCUUACUCCGUUUGCC |
| siRNA 2522 | 2522 | GCAAACGGAGUAAGCCAUA | 8012 | UAUGGCUUACUCCGUUUGC |
| siRNA 2523 | 2523 | CAAACGGAGUAAGCCAUAA | 8013 | UUAUGGCUUACUCCGUUUG |
| siRNA 2524 | 2524 | AAACGGAGUAAGCCAUAAU | 8014 | AUUAUGGCUUACUCCGUUU |
| siRNA 2525 | 2525 | AACGGAGUAAGCCAUAAUA | 8015 | UAUUAUGGCUUACUCCGUU |
| siRNA 2526 | 2526 | ACGGAGUAAGCCAUAAUAU | 8016 | AUAUUAUGGCUUACUCCGU |
| siRNA 2527 | 2527 | CGGAGUAAGCCAUAAUAUU | 8017 | AAUAUUAUGGCUUACUCCG |
| siRNA 2528 | 2528 | GGAGUAAGCCAUAAUAUUA | 8018 | UAAUAUUAUGGCUUACUCC |
| siRNA 2529 | 2529 | GAGUAAGCCAUAAUAUUAC | 8019 | GUAAUAUUAUGGCUUACUC |
| siRNA 2530 | 2530 | AGUAAGCCAUAAUAUUACC | 8020 | GGUAAUAUUAUGGCUUACU |
| siRNA 2531 | 2531 | GUAAGCCAUAAUAUUACCU | 8021 | AGGUAAUAUUAUGGCUUAC |
| siRNA 2532 | 2532 | UAAGCCAUAAUAUUACCUU | 8022 | AAGGUAAUAUUAUGGCUUA |
| siRNA 2533 | 2533 | AAGCCAUAAUAUUACCUUU | 8023 | AAAGGUAAUAUUAUGGCUU |
| siRNA 2534 | 2534 | AGCCAUAAUAUUACCUUUG | 8024 | CAAAGGUAAUAUUAUGGCU |
| siRNA 2535 | 2535 | GCCAUAAUAUUACCUUUGA | 8025 | UCAAAGGUAAUAUUAUGGC |
| siRNA 2536 | 2536 | CCAUAAUAUUACCUUUGAA | 8026 | UUCAAAGGUAAUAUUAUGG |
| siRNA 2537 | 2537 | CAUAAUAUUACCUUUGAAA | 8027 | UUUCAAAGGUAAUAUUAUG |
| siRNA 2538 | 2538 | AUAAUAUUACCUUUGAAAG | 8028 | CUUUCAAAGGUAAUAUUAU |
| siRNA 2539 | 2539 | UAAUAUUACCUUUGAAAGU | 8029 | ACUUUCAAAGGUAAUAUUA |
| siRNA 2540 | 2540 | AAUAUUACCUUUGAAAGUC | 8030 | GACUUUCAAAGGUAAUAUU |
| siRNA 2541 | 2541 | AUAUUACCUUUGAAAGUCC | 8031 | GGACUUUCAAAGGUAAUAU |
| siRNA 2542 | 2542 | UAUUACCUUUGAAAGUCCA | 8032 | UGGACUUUCAAAGGUAAUA |
| siRNA 2543 | 2543 | AUUACCUUUGAAAGUCCAC | 8033 | GUGGACUUUCAAAGGUAAU |
| siRNA 2544 | 2544 | UUACCUUUGAAAGUCCACC | 8034 | GGUGGACUUUCAAAGGUAA |
| siRNA 2545 | 2545 | UACCUUUGAAAGUCCACCU | 8035 | AGGUGGACUUUCAAAGGUA |
| siRNA 2546 | 2546 | ACCUUUGAAAGUCCACCUC | 8036 | GAGGUGGACUUUCAAAGGU |
| siRNA 2547 | 2547 | CCUUUGAAAGUCCACCUCC | 8037 | GGAGGUGGACUUUCAAAGG |
| siRNA 2548 | 2548 | CUUUGAAAGUCCACCUCCA | 8038 | UGGAGGUGGACUUUCAAAG |
| siRNA 2549 | 2549 | UUUGAAAGUCCACCUCCAC | 8039 | GUGGAGGUGGACUUUCAAA |
| siRNA 2550 | 2550 | UUGAAAGUCCACCUCCACC | 8040 | GGUGGAGGUGGACUUUCAA |
| siRNA 2551 | 2551 | UGAAAGUCCACCUCCACCA | 8041 | UGGUGGAGGUGGACUUUCA |
| siRNA 2552 | 2552 | GAAAGUCCACCUCCACCAA | 8042 | UUGGUGGAGGUGGACUUUC |
| siRNA 2553 | 2553 | AAAGUCCACCUCCACCAAU | 8043 | AUUGGUGGAGGUGGACUUU |
| siRNA 2554 | 2554 | AAGUCCACCUCCACCAAUU | 8044 | AAUUGGUGGAGGUGGACUU |
| siRNA 2555 | 2555 | AGUCCACCUCCACCAAUUG | 8045 | CAAUUGGUGGAGGUGGACU |
| siRNA 2556 | 2556 | GUCCACCUCCACCAAUUGA | 8046 | UCAAUUGGUGGAGGUGGAC |
| siRNA 2557 | 2557 | UCCACCUCCACCAAUUGAA | 8047 | UUCAAUUGGUGGAGGUGGA |
| siRNA 2558 | 2558 | CCACCUCCACCAAUUGAAU | 8048 | AUUCAAUUGGUGGAGGUGG |
| siRNA 2559 | 2559 | CACCUCCACCAAUUGAAUG | 8049 | CAUUCAAUUGGUGGAGGUG |
| siRNA 2560 | 2560 | ACCUCCACCAAUUGAAUGG | 8050 | CCAUUCAAUUGGUGGAGGU |
| siRNA 2561 | 2561 | CCUCCACCAAUUGAAUGGC | 8051 | GCCAUUCAAUUGGUGGAGG |
| siRNA 2562 | 2562 | CUCCACCAAUUGAAUGGCA | 8052 | UGCCAUUCAAUUGGUGGAG |
| siRNA 2563 | 2563 | UCCACCAAUUGAAUGGCAU | 8053 | AUGCCAUUCAAUUGGUGGA |
| siRNA 2564 | 2564 | CCACCAAUUGAAUGGCAUA | 8054 | UAUGCCAUUCAAUUGGUGG |
| siRNA 2565 | 2565 | CACCAAUUGAAUGGCAUAU | 8055 | AUAUGCCAUUCAAUUGGUG |
| siRNA 2566 | 2566 | ACCAAUUGAAUGGCAUAUC | 8056 | GAUAUGCCAUUCAAUUGGU |
| siRNA 2567 | 2567 | CCAAUUGAAUGGCAUAUCA | 8057 | UGAUAUGCCAUUCAAUUGG |
| siRNA 2568 | 2568 | CAAUUGAAUGGCAUAUCAG | 8058 | CUGAUAUGCCAUUCAAUUG |
| siRNA 2569 | 2569 | AAUUGAAUGGCAUAUCAGC | 8059 | GCUGAUAUGCCAUUCAAUU |
| siRNA 2570 | 2570 | AUUGAAUGGCAUAUCAGCA | 8060 | UGCUGAUAUGCCAUUCAAU |
| siRNA 2571 | 2571 | UUGAAUGGCAUAUCAGCAA | 8061 | UUGCUGAUAUGCCAUUCAA |
| siRNA 2572 | 2572 | UGAAUGGCAUAUCAGCAAA | 8062 | UUUGCUGAUAUGCCAUUCA |
| siRNA 2573 | 2573 | GAAUGGCAUAUCAGCAAAC | 8063 | GUUUGCUGAUAUGCCAUUC |
| siRNA 2574 | 2574 | AAUGGCAUAUCAGCAAACC | 8064 | GGUUUGCUGAUAUGCCAUU |
| siRNA 2575 | 2575 | AUGGCAUAUCAGCAAACCA | 8065 | UGGUUUGCUGAUAUGCCAU |
| siRNA 2576 | 2576 | UGGCAUAUCAGCAAACCAG | 8066 | CUGGUUUGCUGAUAUGCCA |
| siRNA 2577 | 2577 | GGCAUAUCAGCAAACCAGG | 8067 | CCUGGUUUGCUGAUAUGCC |
| siRNA 2578 | 2578 | GCAUAUCAGCAAACCAGGA | 8068 | UCCUGGUUUGCUGAUAUGC |
| siRNA 2579 | 2579 | CAUAUCAGCAAACCAGGAC | 8069 | GUCCUGGUUUGCUGAUAUG |
| siRNA 2580 | 2580 | AUAUCAGCAAACCAGGACA | 8070 | UGUCCUGGUUUGCUGAUAU |
| siRNA 2581 | 2581 | UAUCAGCAAACCAGGACAG | 8071 | CUGUCCUGGUUUGCUGAUA |
| siRNA 2582 | 2582 | AUCAGCAAACCAGGACAGU | 8072 | ACUGUCCUGGUUUGCUGAU |
| siRNA 2583 | 2583 | UCAGCAAACCAGGACAGUU | 8073 | AACUGUCCUGGUUUGCUGA |
| siRNA 2584 | 2584 | CAGCAAACCAGGACAGUUU | 8074 | AAACUGUCCUGGUUUGCUG |
| siRNA 2585 | 2585 | AGCAAACCAGGACAGUUUG | 8075 | CAAACUGUCCUGGUUUGCU |
| siRNA 2586 | 2586 | GCAAACCAGGACAGUUUGA | 8076 | UCAAACUGUCCUGGUUUGC |
| siRNA 2587 | 2587 | CAAACCAGGACAGUUUGAA | 8077 | UUCAAACUGUCCUGGUUUG |
| siRNA 2588 | 2588 | AAACCAGGACAGUUUGAAA | 8078 | UUUCAAACUGUCCUGGUUU |
| siRNA 2589 | 2589 | AACCAGGACAGUUUGAAAC | 8079 | GUUUCAAACUGUCCUGGUU |
| siRNA 2590 | 2590 | ACCAGGACAGUUUGAAACA | 8080 | UGUUUCAAACUGUCCUGGU |
| siRNA 2591 | 2591 | CCAGGACAGUUUGAAACAU | 8081 | AUGUUUCAAACUGUCCUGG |
| siRNA 2592 | 2592 | CAGGACAGUUUGAAACAUU | 8082 | AAUGUUUCAAACUGUCCUG |
| siRNA 2593 | 2593 | AGGACAGUUUGAAACAUUU | 8083 | AAAUGUUUCAAACUGUCCU |
| siRNA 2594 | 2594 | GGACAGUUUGAAACAUUUG | 8084 | CAAAUGUUUCAAACUGUCC |
| siRNA 2595 | 2595 | GACAGUUUGAAACAUUUGA | 8085 | UCAAAUGUUUCAAACUGUC |
| siRNA 2596 | 2596 | ACAGUUUGAAACAUUUGAU | 8086 | AUCAAAUGUUUCAAACUGU |
| siRNA 2597 | 2597 | CAGUUUGAAACAUUUGAUC | 8087 | GAUCAAAUGUUUCAAACUG |
| siRNA 2598 | 2598 | AGUUUGAAACAUUUGAUCU | 8088 | AGAUCAAAUGUUUCAAACU |
| siRNA 2599 | 2599 | GUUUGAAACAUUUGAUCUC | 8089 | GAGAUCAAAUGUUUCAAAC |
| siRNA 2600 | 2600 | UUUGAAACAUUUGAUCUCA | 8090 | UGAGAUCAAAUGUUUCAAA |
| siRNA 2601 | 2601 | UUGAAACAUUUGAUCUCAU | 8091 | AUGAGAUCAAAUGUUUCAA |
| siRNA 2602 | 2602 | UGAAACAUUUGAUCUCAUG | 8092 | CAUGAGAUCAAAUGUUUCA |
| siRNA 2603 | 2603 | GAAACAUUUGAUCUCAUGA | 8093 | UCAUGAGAUCAAAUGUUUC |
| siRNA 2604 | 2604 | AAACAUUUGAUCUCAUGAC | 8094 | GUCAUGAGAUCAAAUGUUU |
| siRNA 2605 | 2605 | AACAUUUGAUCUCAUGACA | 8095 | UGUCAUGAGAUCAAAUGUU |
| siRNA 2606 | 2606 | ACAUUUGAUCUCAUGACAC | 8096 | GUGUCAUGAGAUCAAAUGU |
| siRNA 2607 | 2607 | CAUUUGAUCUCAUGACACU | 8097 | AGUGUCAUGAGAUCAAAUG |
| siRNA 2608 | 2608 | AUUUGAUCUCAUGACACUU | 8098 | AAGUGUCAUGAGAUCAAAU |
| siRNA 2609 | 2609 | UUUGAUCUCAUGACACUUC | 8099 | GAAGUGUCAUGAGAUCAAA |
| siRNA 2610 | 2610 | UUGAUCUCAUGACACUUCA | 8100 | UGAAGUGUCAUGAGAUCAA |
| siRNA 2611 | 2611 | UGAUCUCAUGACACUUCAU | 8101 | AUGAAGUGUCAUGAGAUCA |
| siRNA 2612 | 2612 | GAUCUCAUGACACUUCAUC | 8102 | GAUGAAGUGUCAUGAGAUC |
| siRNA 2613 | 2613 | AUCUCAUGACACUUCAUCC | 8103 | GGAUGAAGUGUCAUGAGAU |
| siRNA 2614 | 2614 | UCUCAUGACACUUCAUCCA | 8104 | UGGAUGAAGUGUCAUGAGA |
| siRNA 2615 | 2615 | CUCAUGACACUUCAUCCAA | 8105 | UUGGAUGAAGUGUCAUGAG |
| siRNA 2616 | 2616 | UCAUGACACUUCAUCCAAU | 8106 | AUUGGAUGAAGUGUCAUGA |
| siRNA 2617 | 2617 | CAUGACACUUCAUCCAAUA | 8107 | UAUUGGAUGAAGUGUCAUG |
| siRNA 2618 | 2618 | AUGACACUUCAUCCAAUAG | 8108 | CUAUUGGAUGAAGUGUCAU |
| siRNA 2619 | 2619 | UGACACUUCAUCCAAUAGA | 8109 | UCUAUUGGAUGAAGUGUCA |
| siRNA 2620 | 2620 | GACACUUCAUCCAAUAGAA | 8110 | UUCUAUUGGAUGAAGUGUC |
| siRNA 2621 | 2621 | ACACUUCAUCCAAUAGAAA | 8111 | UUUCUAUUGGAUGAAGUGU |
| siRNA 2622 | 2622 | CACUUCAUCCAAUAGAAAU | 8112 | AUUUCUAUUGGAUGAAGUG |
| siRNA 2623 | 2623 | ACUUCAUCCAAUAGAAAUU | 8113 | AAUUUCUAUUGGAUGAAGU |
| siRNA 2624 | 2624 | CUUCAUCCAAUAGAAAUUG | 8114 | CAAUUUCUAUUGGAUGAAG |
| siRNA 2625 | 2625 | UUCAUCCAAUAGAAAUUGC | 8115 | GCAAUUUCUAUUGGAUGAA |
| siRNA 2626 | 2626 | UCAUCCAAUAGAAAUUGCA | 8116 | UGCAAUUUCUAUUGGAUGA |
| siRNA 2627 | 2627 | CAUCCAAUAGAAAUUGCAC | 8117 | GUGCAAUUUCUAUUGGAUG |
| siRNA 2628 | 2628 | AUCCAAUAGAAAUUGCACG | 8118 | CGUGCAAUUUCUAUUGGAU |
| siRNA 2629 | 2629 | UCCAAUAGAAAUUGCACGU | 8119 | ACGUGCAAUUUCUAUUGGA |
| siRNA 2630 | 2630 | CCAAUAGAAAUUGCACGUC | 8120 | GACGUGCAAUUUCUAUUGG |
| siRNA 2631 | 2631 | CAAUAGAAAUUGCACGUCA | 8121 | UGACGUGCAAUUUCUAUUG |
| siRNA 2632 | 2632 | AAUAGAAAUUGCACGUCAG | 8122 | CUGACGUGCAAUUUCUAUU |
| siRNA 2633 | 2633 | AUAGAAAUUGCACGUCAGC | 8123 | GCUGACGUGCAAUUUCUAU |
| siRNA 2634 | 2634 | UAGAAAUUGCACGUCAGCU | 8124 | AGCUGACGUGCAAUUUCUA |
| siRNA 2635 | 2635 | AGAAAUUGCACGUCAGCUG | 8125 | CAGCUGACGUGCAAUUUCU |
| siRNA 2636 | 2636 | GAAAUUGCACGUCAGCUGA | 8126 | UCAGCUGACGUGCAAUUUC |
| siRNA 2637 | 2637 | AAAUUGCACGUCAGCUGAC | 8127 | GUCAGCUGACGUGCAAUUU |
| siRNA 2638 | 2638 | AAUUGCACGUCAGCUGACA | 8128 | UGUCAGCUGACGUGCAAUU |
| siRNA 2639 | 2639 | AUUGCACGUCAGCUGACAC | 8129 | GUGUCAGCUGACGUGCAAU |
| siRNA 2640 | 2640 | UUGCACGUCAGCUGACACU | 8130 | AGUGUCAGCUGACGUGCAA |
| siRNA 2641 | 2641 | UGCACGUCAGCUGACACUU | 8131 | AAGUGUCAGCUGACGUGCA |
| siRNA 2642 | 2642 | GCACGUCAGCUGACACUUU | 8132 | AAAGUGUCAGCUGACGUGC |
| siRNA 2643 | 2643 | CACGUCAGCUGACACUUUU | 8133 | AAAAGUGUCAGCUGACGUG |
| siRNA 2644 | 2644 | ACGUCAGCUGACACUUUUG | 8134 | CAAAAGUGUCAGCUGACGU |
| siRNA 2645 | 2645 | CGUCAGCUGACACUUUUGG | 8135 | CCAAAAGUGUCAGCUGACG |
| siRNA 2646 | 2646 | GUCAGCUGACACUUUUGGA | 8136 | UCCAAAAGUGUCAGCUGAC |
| siRNA 2647 | 2647 | UCAGCUGACACUUUUGGAG | 8137 | CUCCAAAAGUGUCAGCUGA |
| siRNA 2648 | 2648 | CAGCUGACACUUUUGGAGU | 8138 | ACUCCAAAAGUGUCAGCUG |
| siRNA 2649 | 2649 | AGCUGACACUUUUGGAGUC | 8139 | GACUCCAAAAGUGUCAGCU |
| siRNA 2650 | 2650 | GCUGACACUUUUGGAGUCU | 8140 | AGACUCCAAAAGUGUCAGC |
| siRNA 2651 | 2651 | CUGACACUUUUGGAGUCUG | 8141 | CAGACUCCAAAAGUGUCAG |
| siRNA 2652 | 2652 | UGACACUUUUGGAGUCUGA | 8142 | UCAGACUCCAAAAGUGUCA |
| siRNA 2653 | 2653 | GACACUUUUGGAGUCUGAU | 8143 | AUCAGACUCCAAAAGUGUC |
| siRNA 2654 | 2654 | ACACUUUUGGAGUCUGAUC | 8144 | GAUCAGACUCCAAAAGUGU |
| siRNA 2655 | 2655 | CACUUUUGGAGUCUGAUCU | 8145 | AGAUCAGACUCCAAAAGUG |
| siRNA 2656 | 2656 | ACUUUUGGAGUCUGAUCUU | 8146 | AAGAUCAGACUCCAAAAGU |
| siRNA 2657 | 2657 | CUUUUGGAGUCUGAUCUUU | 8147 | AAAGAUCAGACUCCAAAAG |
| siRNA 2658 | 2658 | UUUUGGAGUCUGAUCUUUA | 8148 | UAAAGAUCAGACUCCAAAA |
| siRNA 2659 | 2659 | UUUGGAGUCUGAUCUUUAC | 8149 | GUAAAGAUCAGACUCCAAA |
| siRNA 2660 | 2660 | UUGGAGUCUGAUCUUUACA | 8150 | UGUAAAGAUCAGACUCCAA |
| siRNA 2661 | 2661 | UGGAGUCUGAUCUUUACAG | 8151 | CUGUAAAGAUCAGACUCCA |
| siRNA 2662 | 2662 | GGAGUCUGAUCUUUACAGG | 8152 | CCUGUAAAGAUCAGACUCC |
| siRNA 2663 | 2663 | GAGUCUGAUCUUUACAGGA | 8153 | UCCUGUAAAGAUCAGACUC |
| siRNA 2664 | 2664 | AGUCUGAUCUUUACAGGAA | 8154 | UUCCUGUAAAGAUCAGACU |
| siRNA 2665 | 2665 | GUCUGAUCUUUACAGGAAA | 8155 | UUUCCUGUAAAGAUCAGAC |
| siRNA 2666 | 2666 | UCUGAUCUUUACAGGAAAG | 8156 | CUUUCCUGUAAAGAUCAGA |
| siRNA 2667 | 2667 | CUGAUCUUUACAGGAAAGU | 8157 | ACUUUCCUGUAAAGAUCAG |
| siRNA 2668 | 2668 | UGAUCUUUACAGGAAAGUU | 8158 | AACUUUCCUGUAAAGAUCA |
| siRNA 2669 | 2669 | GAUCUUUACAGGAAAGUUC | 8159 | GAACUUUCCUGUAAAGAUC |
| siRNA 2670 | 2670 | AUCUUUACAGGAAAGUUCA | 8160 | UGAACUUUCCUGUAAAGAU |
| siRNA 2671 | 2671 | UCUUUACAGGAAAGUUCAA | 8161 | UUGAACUUUCCUGUAAAGA |
| siRNA 2672 | 2672 | CUUUACAGGAAAGUUCAAC | 8162 | GUUGAACUUUCCUGUAAAG |
| siRNA 2673 | 2673 | UUUACAGGAAAGUUCAACC | 8163 | GGUUGAACUUUCCUGUAAA |
| siRNA 2674 | 2674 | UUACAGGAAAGUUCAACCG | 8164 | CGGUUGAACUUUCCUGUAA |
| siRNA 2675 | 2675 | UACAGGAAAGUUCAACCGU | 8165 | ACGGUUGAACUUUCCUGUA |
| siRNA 2676 | 2676 | ACAGGAAAGUUCAACCGUC | 8166 | GACGGUUGAACUUUCCUGU |
| siRNA 2677 | 2677 | CAGGAAAGUUCAACCGUCU | 8167 | AGACGGUUGAACUUUCCUG |
| siRNA 2678 | 2678 | AGGAAAGUUCAACCGUCUG | 8168 | CAGACGGUUGAACUUUCCU |
| siRNA 2679 | 2679 | GGAAAGUUCAACCGUCUGA | 8169 | UCAGACGGUUGAACUUUCC |
| siRNA 2680 | 2680 | GAAAGUUCAACCGUCUGAA | 8170 | UUCAGACGGUUGAACUUUC |
| siRNA 2681 | 2681 | AAAGUUCAACCGUCUGAAC | 8171 | GUUCAGACGGUUGAACUUU |
| siRNA 2682 | 2682 | AAGUUCAACCGUCUGAACU | 8172 | AGUUCAGACGGUUGAACUU |
| siRNA 2683 | 2683 | AGUUCAACCGUCUGAACUU | 8173 | AAGUUCAGACGGUUGAACU |
| siRNA 2684 | 2684 | GUUCAACCGUCUGAACUUG | 8174 | CAAGUUCAGACGGUUGAAC |
| siRNA 2685 | 2685 | UUCAACCGUCUGAACUUGU | 8175 | ACAAGUUCAGACGGUUGAA |
| siRNA 2686 | 2686 | UCAACCGUCUGAACUUGUA | 8176 | UACAAGUUCAGACGGUUGA |
| siRNA 2687 | 2687 | CAACCGUCUGAACUUGUAG | 8177 | CUACAAGUUCAGACGGUUG |
| siRNA 2688 | 2688 | AACCGUCUGAACUUGUAGG | 8178 | CCUACAAGUUCAGACGGUU |
| siRNA 2689 | 2689 | ACCGUCUGAACUUGUAGGG | 8179 | CCCUACAAGUUCAGACGGU |
| siRNA 2690 | 2690 | CCGUCUGAACUUGUAGGGA | 8180 | UCCCUACAAGUUCAGACGG |
| siRNA 2691 | 2691 | CGUCUGAACUUGUAGGGAG | 8181 | CUCCCUACAAGUUCAGACG |
| siRNA 2692 | 2692 | GUCUGAACUUGUAGGGAGU | 8182 | ACUCCCUACAAGUUCAGAC |
| siRNA 2693 | 2693 | UCUGAACUUGUAGGGAGUG | 8183 | CACUCCCUACAAGUUCAGA |
| siRNA 2694 | 2694 | CUGAACUUGUAGGGAGUGU | 8184 | ACACUCCCUACAAGUUCAG |
| siRNA 2695 | 2695 | UGAACUUGUAGGGAGUGUG | 8185 | CACACUCCCUACAAGUUCA |
| siRNA 2696 | 2696 | GAACUUGUAGGGAGUGUGU | 8186 | ACACACUCCCUACAAGUUC |
| siRNA 2697 | 2697 | AACUUGUAGGGAGUGUGUG | 8187 | CACACACUCCCUACAAGUU |
| siRNA 2698 | 2698 | ACUUGUAGGGAGUGUGUGG | 8188 | CCACACACUCCCUACAAGU |
| siRNA 2699 | 2699 | CUUGUAGGGAGUGUGUGGA | 8189 | UCCACACACUCCCUACAAG |
| siRNA 2700 | 2700 | UUGUAGGGAGUGUGUGGAC | 8190 | GUCCACACACUCCCUACAA |
| siRNA 2701 | 2701 | UGUAGGGAGUGUGUGGACC | 8191 | GGUCCACACACUCCCUACA |
| siRNA 2702 | 2702 | GUAGGGAGUGUGUGGACCA | 8192 | UGGUCCACACACUCCCUAC |
| siRNA 2703 | 2703 | UAGGGAGUGUGUGGACCAA | 8193 | UUGGUCCACACACUCCCUA |
| siRNA 2704 | 2704 | AGGGAGUGUGUGGACCAAA | 8194 | UUUGGUCCACACACUCCCU |
| siRNA 2705 | 2705 | GGGAGUGUGUGGACCAAAG | 8195 | CUUUGGUCCACACACUCCC |
| siRNA 2706 | 2706 | GGAGUGUGUGGACCAAAGA | 8196 | UCUUUGGUCCACACACUCC |
| siRNA 2707 | 2707 | GAGUGUGUGGACCAAAGAA | 8197 | UUCUUUGGUCCACACACUC |
| siRNA 2708 | 2708 | AGUGUGUGGACCAAAGAAG | 8198 | CUUCUUUGGUCCACACACU |
| siRNA 2709 | 2709 | GUGUGUGGACCAAAGAAGA | 8199 | UCUUCUUUGGUCCACACAC |
| siRNA 2710 | 2710 | UGUGUGGACCAAAGAAGAU | 8200 | AUCUUCUUUGGUCCACACA |
| siRNA 2711 | 2711 | GUGUGGACCAAAGAAGAUA | 8201 | UAUCUUCUUUGGUCCACAC |
| siRNA 2712 | 2712 | UGUGGACCAAAGAAGAUAA | 8202 | UUAUCUUCUUUGGUCCACA |
| siRNA 2713 | 2713 | GUGGACCAAAGAAGAUAAA | 8203 | UUUAUCUUCUUUGGUCCAC |
| siRNA 2714 | 2714 | UGGACCAAAGAAGAUAAAG | 8204 | CUUUAUCUUCUUUGGUCCA |
| siRNA 2715 | 2715 | GGACCAAAGAAGAUAAAGA | 8205 | UCUUUAUCUUCUUUGGUCC |
| siRNA 2716 | 2716 | GACCAAAGAAGAUAAAGAA | 8206 | UUCUUUAUCUUCUUUGGUC |
| siRNA 2717 | 2717 | ACCAAAGAAGAUAAAGAAA | 8207 | UUUCUUUAUCUUCUUUGGU |
| siRNA 2718 | 2718 | CCAAAGAAGAUAAAGAAAU | 8208 | AUUUCUUUAUCUUCUUUGG |
| siRNA 2719 | 2719 | CAAAGAAGAUAAAGAAAUA | 8209 | UAUUUCUUUAUCUUCUUUG |
| siRNA 2720 | 2720 | AAAGAAGAUAAAGAAAUAA | 8210 | UUAUUUCUUUAUCUUCUUU |
| siRNA 2721 | 2721 | AAGAAGAUAAAGAAAUAAA | 8211 | UUUAUUUCUUUAUCUUCUU |
| siRNA 2722 | 2722 | AGAAGAUAAAGAAAUAAAU | 8212 | AUUUAUUUCUUUAUCUUCU |
| siRNA 2723 | 2723 | GAAGAUAAAGAAAUAAAUU | 8213 | AAUUUAUUUCUUUAUCUUC |
| siRNA 2724 | 2724 | AAGAUAAAGAAAUAAAUUC | 8214 | GAAUUUAUUUCUUUAUCUU |
| siRNA 2725 | 2725 | AGAUAAAGAAAUAAAUUCU | 8215 | AGAAUUUAUUUCUUUAUCU |
| siRNA 2726 | 2726 | GAUAAAGAAAUAAAUUCUC | 8216 | GAGAAUUUAUUUCUUUAUC |
| siRNA 2727 | 2727 | AUAAAGAAAUAAAUUCUCC | 8217 | GGAGAAUUUAUUUCUUUAU |
| siRNA 2728 | 2728 | UAAAGAAAUAAAUUCUCCA | 8218 | UGGAGAAUUUAUUUCUUUA |
| siRNA 2729 | 2729 | AAAGAAAUAAAUUCUCCAA | 8219 | UUGGAGAAUUUAUUUCUUU |
| siRNA 2730 | 2730 | AAGAAAUAAAUUCUCCAAA | 8220 | UUUGGAGAAUUUAUUUCUU |
| siRNA 2731 | 2731 | AGAAAUAAAUUCUCCAAAU | 8221 | AUUUGGAGAAUUUAUUUCU |
| siRNA 2732 | 2732 | GAAAUAAAUUCUCCAAAUU | 8222 | AAUUUGGAGAAUUUAUUUC |
| siRNA 2733 | 2733 | AAAUAAAUUCUCCAAAUUU | 8223 | AAAUUUGGAGAAUUUAUUU |
| siRNA 2734 | 2734 | AAUAAAUUCUCCAAAUUUA | 8224 | UAAAUUUGGAGAAUUUAUU |
| siRNA 2735 | 2735 | AUAAAUUCUCCAAAUUUAU | 8225 | AUAAAUUUGGAGAAUUUAU |
| siRNA 2736 | 2736 | UAAAUUCUCCAAAUUUAUU | 8226 | AAUAAAUUUGGAGAAUUUA |
| siRNA 2737 | 2737 | AAAUUCUCCAAAUUUAUUA | 8227 | UAAUAAAUUUGGAGAAUUU |
| siRNA 2738 | 2738 | AAUUCUCCAAAUUUAUUAA | 8228 | UUAAUAAAUUUGGAGAAUU |
| siRNA 2739 | 2739 | AUUCUCCAAAUUUAUUAAA | 8229 | UUUAAUAAAUUUGGAGAAU |
| siRNA 2740 | 2740 | UUCUCCAAAUUUAUUAAAA | 8230 | UUUUAAUAAAUUUGGAGAA |
| siRNA 2741 | 2741 | UCUCCAAAUUUAUUAAAAA | 8231 | UUUUUAAUAAAUUUGGAGA |
| siRNA 2742 | 2742 | CUCCAAAUUUAUUAAAAAU | 8232 | AUUUUUAAUAAAUUUGGAG |
| siRNA 2743 | 2743 | UCCAAAUUUAUUAAAAAUG | 8233 | CAUUUUUAAUAAAUUUGGA |
| siRNA 2744 | 2744 | CCAAAUUUAUUAAAAAUGA | 8234 | UCAUUUUUAAUAAAUUUGG |
| siRNA 2745 | 2745 | CAAAUUUAUUAAAAAUGAU | 8235 | AUCAUUUUUAAUAAAUUUG |
| siRNA 2746 | 2746 | AAAUUUAUUAAAAAUGAUU | 8236 | AAUCAUUUUUAAUAAAUUU |
| siRNA 2747 | 2747 | AAUUUAUUAAAAAUGAUUC | 8237 | GAAUCAUUUUUAAUAAAUU |
| siRNA 2748 | 2748 | AUUUAUUAAAAAUGAUUCG | 8238 | CGAAUCAUUUUUAAUAAAU |
| siRNA 2749 | 2749 | UUUAUUAAAAAUGAUUCGC | 8239 | GCGAAUCAUUUUUAAUAAA |
| siRNA 2750 | 2750 | UUAUUAAAAAUGAUUCGCC | 8240 | GGCGAAUCAUUUUUAAUAA |
| siRNA 2751 | 2751 | UAUUAAAAAUGAUUCGCCA | 8241 | UGGCGAAUCAUUUUUAAUA |
| siRNA 2752 | 2752 | AUUAAAAAUGAUUCGCCAU | 8242 | AUGGCGAAUCAUUUUUAAU |
| siRNA 2753 | 2753 | UUAAAAAUGAUUCGCCAUA | 8243 | UAUGGCGAAUCAUUUUUAA |
| siRNA 2754 | 2754 | UAAAAAUGAUUCGCCAUAC | 8244 | GUAUGGCGAAUCAUUUUUA |
| siRNA 2755 | 2755 | AAAAAUGAUUCGCCAUACC | 8245 | GGUAUGGCGAAUCAUUUUU |
| siRNA 2756 | 2756 | AAAAUGAUUCGCCAUACCA | 8246 | UGGUAUGGCGAAUCAUUUU |
| siRNA 2757 | 2757 | AAAUGAUUCGCCAUACCAC | 8247 | GUGGUAUGGCGAAUCAUUU |
| siRNA 2758 | 2758 | AAUGAUUCGCCAUACCACA | 8248 | UGUGGUAUGGCGAAUCAUU |
| siRNA 2759 | 2759 | AUGAUUCGCCAUACCACAA | 8249 | UUGUGGUAUGGCGAAUCAU |
| siRNA 2760 | 2760 | UGAUUCGCCAUACCACAAA | 8250 | UUUGUGGUAUGGCGAAUCA |
| siRNA 2761 | 2761 | GAUUCGCCAUACCACAAAU | 8251 | AUUUGUGGUAUGGCGAAUC |
| siRNA 2762 | 2762 | AUUCGCCAUACCACAAAUC | 8252 | GAUUUGUGGUAUGGCGAAU |
| siRNA 2763 | 2763 | UUCGCCAUACCACAAAUCU | 8253 | AGAUUUGUGGUAUGGCGAA |
| siRNA 2764 | 2764 | UCGCCAUACCACAAAUCUC | 8254 | GAGAUUUGUGGUAUGGCGA |
| siRNA 2765 | 2765 | CGCCAUACCACAAAUCUCA | 8255 | UGAGAUUUGUGGUAUGGCG |
| siRNA 2766 | 2766 | GCCAUACCACAAAUCUCAC | 8256 | GUGAGAUUUGUGGUAUGGC |
| siRNA 2767 | 2767 | CCAUACCACAAAUCUCACC | 8257 | GGUGAGAUUUGUGGUAUGG |
| siRNA 2768 | 2768 | CAUACCACAAAUCUCACCC | 8258 | GGGUGAGAUUUGUGGUAUG |
| siRNA 2769 | 2769 | AUACCACAAAUCUCACCCU | 8259 | AGGGUGAGAUUUGUGGUAU |
| siRNA 2770 | 2770 | UACCACAAAUCUCACCCUC | 8260 | GAGGGUGAGAUUUGUGGUA |
| siRNA 2771 | 2771 | ACCACAAAUCUCACCCUCU | 8261 | AGAGGGUGAGAUUUGUGGU |
| siRNA 2772 | 2772 | CCACAAAUCUCACCCUCUG | 8262 | CAGAGGGUGAGAUUUGUGG |
| siRNA 2773 | 2773 | CACAAAUCUCACCCUCUGG | 8263 | CCAGAGGGUGAGAUUUGUG |
| siRNA 2774 | 2774 | ACAAAUCUCACCCUCUGGU | 8264 | ACCAGAGGGUGAGAUUUGU |
| siRNA 2775 | 2775 | CAAAUCUCACCCUCUGGUU | 8265 | AACCAGAGGGUGAGAUUUG |
| siRNA 2776 | 2776 | AAAUCUCACCCUCUGGUUU | 8266 | AAACCAGAGGGUGAGAUUU |
| siRNA 2777 | 2777 | AAUCUCACCCUCUGGUUUG | 8267 | CAAACCAGAGGGUGAGAUU |
| siRNA 2778 | 2778 | AUCUCACCCUCUGGUUUGA | 8268 | UCAAACCAGAGGGUGAGAU |
| siRNA 2779 | 2779 | UCUCACCCUCUGGUUUGAA | 8269 | UUCAAACCAGAGGGUGAGA |
| siRNA 2780 | 2780 | CUCACCCUCUGGUUUGAAA | 8270 | UUUCAAACCAGAGGGUGAG |
| siRNA 2781 | 2781 | UCACCCUCUGGUUUGAAAA | 8271 | UUUUCAAACCAGAGGGUGA |
| siRNA 2782 | 2782 | CACCCUCUGGUUUGAAAAA | 8272 | UUUUUCAAACCAGAGGGUG |
| siRNA 2783 | 2783 | ACCCUCUGGUUUGAAAAAU | 8273 | AUUUUUCAAACCAGAGGGU |
| siRNA 2784 | 2784 | CCCUCUGGUUUGAAAAAUG | 8274 | CAUUUUUCAAACCAGAGGG |
| siRNA 2785 | 2785 | CCUCUGGUUUGAAAAAUGC | 8275 | GCAUUUUUCAAACCAGAGG |
| siRNA 2786 | 2786 | CUCUGGUUUGAAAAAUGCA | 8276 | UGCAUUUUUCAAACCAGAG |
| siRNA 2787 | 2787 | UCUGGUUUGAAAAAUGCAU | 8277 | AUGCAUUUUUCAAACCAGA |
| siRNA 2788 | 2788 | CUGGUUUGAAAAAUGCAUU | 8278 | AAUGCAUUUUUCAAACCAG |
| siRNA 2789 | 2789 | UGGUUUGAAAAAUGCAUUG | 8279 | CAAUGCAUUUUUCAAACCA |
| siRNA 2790 | 2790 | GGUUUGAAAAAUGCAUUGU | 8280 | ACAAUGCAUUUUUCAAACC |
| siRNA 2791 | 2791 | GUUUGAAAAAUGCAUUGUG | 8281 | CACAAUGCAUUUUUCAAAC |
| siRNA 2792 | 2792 | UUUGAAAAAUGCAUUGUGG | 8282 | CCACAAUGCAUUUUUCAAA |
| siRNA 2793 | 2793 | UUGAAAAAUGCAUUGUGGA | 8283 | UCCACAAUGCAUUUUUCAA |
| siRNA 2794 | 2794 | UGAAAAAUGCAUUGUGGAA | 8284 | UUCCACAAUGCAUUUUUCA |
| siRNA 2795 | 2795 | GAAAAAUGCAUUGUGGAAG | 8285 | CUUCCACAAUGCAUUUUUC |
| siRNA 2796 | 2796 | AAAAAUGCAUUGUGGAAGC | 8286 | GCUUCCACAAUGCAUUUUU |
| siRNA 2797 | 2797 | AAAAUGCAUUGUGGAAGCA | 8287 | UGCUUCCACAAUGCAUUUU |
| siRNA 2798 | 2798 | AAAUGCAUUGUGGAAGCAG | 8288 | CUGCUUCCACAAUGCAUUU |
| siRNA 2799 | 2799 | AAUGCAUUGUGGAAGCAGA | 8289 | UCUGCUUCCACAAUGCAUU |
| siRNA 2800 | 2800 | AUGCAUUGUGGAAGCAGAA | 8290 | UUCUGCUUCCACAAUGCAU |
| siRNA 2801 | 2801 | UGCAUUGUGGAAGCAGAAA | 8291 | UUUCUGCUUCCACAAUGCA |
| siRNA 2802 | 2802 | GCAUUGUGGAAGCAGAAAA | 8292 | UUUUCUGCUUCCACAAUGC |
| siRNA 2803 | 2803 | CAUUGUGGAAGCAGAAAAU | 8293 | AUUUUCUGCUUCCACAAUG |
| siRNA 2804 | 2804 | AUUGUGGAAGCAGAAAAUU | 8294 | AAUUUUCUGCUUCCACAAU |
| siRNA 2805 | 2805 | UUGUGGAAGCAGAAAAUUU | 8295 | AAAUUUUCUGCUUCCACAA |
| siRNA 2806 | 2806 | UGUGGAAGCAGAAAAUUUU | 8296 | AAAAUUUUCUGCUUCCACA |
| siRNA 2807 | 2807 | GUGGAAGCAGAAAAUUUUG | 8297 | CAAAAUUUUCUGCUUCCAC |
| siRNA 2808 | 2808 | UGGAAGCAGAAAAUUUUGA | 8298 | UCAAAAUUUUCUGCUUCCA |
| siRNA 2809 | 2809 | GGAAGCAGAAAAUUUUGAA | 8299 | UUCAAAAUUUUCUGCUUCC |
| siRNA 2810 | 2810 | GAAGCAGAAAAUUUUGAAG | 8300 | CUUCAAAAUUUUCUGCUUC |
| siRNA 2811 | 2811 | AAGCAGAAAAUUUUGAAGA | 8301 | UCUUCAAAAUUUUCUGCUU |
| siRNA 2812 | 2812 | AGCAGAAAAUUUUGAAGAA | 8302 | UUCUUCAAAAUUUUCUGCU |
| siRNA 2813 | 2813 | GCAGAAAAUUUUGAAGAAC | 8303 | GUUCUUCAAAAUUUUCUGC |
| siRNA 2814 | 2814 | CAGAAAAUUUUGAAGAACG | 8304 | CGUUCUUCAAAAUUUUCUG |
| siRNA 2815 | 2815 | AGAAAAUUUUGAAGAACGG | 8305 | CCGUUCUUCAAAAUUUUCU |
| siRNA 2816 | 2816 | GAAAAUUUUGAAGAACGGG | 8306 | CCCGUUCUUCAAAAUUUUC |
| siRNA 2817 | 2817 | AAAAUUUUGAAGAACGGGU | 8307 | ACCCGUUCUUCAAAAUUUU |
| siRNA 2818 | 2818 | AAAUUUUGAAGAACGGGUG | 8308 | CACCCGUUCUUCAAAAUUU |
| siRNA 2819 | 2819 | AAUUUUGAAGAACGGGUGG | 8309 | CCACCCGUUCUUCAAAAUU |
| siRNA 2820 | 2820 | AUUUUGAAGAACGGGUGGC | 8310 | GCCACCCGUUCUUCAAAAU |
| siRNA 2821 | 2821 | UUUUGAAGAACGGGUGGCA | 8311 | UGCCACCCGUUCUUCAAAA |
| siRNA 2822 | 2822 | UUUGAAGAACGGGUGGCAG | 8312 | CUGCCACCCGUUCUUCAAA |
| siRNA 2823 | 2823 | UUGAAGAACGGGUGGCAGU | 8313 | ACUGCCACCCGUUCUUCAA |
| siRNA 2824 | 2824 | UGAAGAACGGGUGGCAGUA | 8314 | UACUGCCACCCGUUCUUCA |
| siRNA 2825 | 2825 | GAAGAACGGGUGGCAGUAC | 8315 | GUACUGCCACCCGUUCUUC |
| siRNA 2826 | 2826 | AAGAACGGGUGGCAGUACU | 8316 | AGUACUGCCACCCGUUCUU |
| siRNA 2827 | 2827 | AGAACGGGUGGCAGUACUA | 8317 | UAGUACUGCCACCCGUUCU |
| siRNA 2828 | 2828 | GAACGGGUGGCAGUACUAA | 8318 | UUAGUACUGCCACCCGUUC |
| siRNA 2829 | 2829 | AACGGGUGGCAGUACUAAG | 8319 | CUUAGUACUGCCACCCGUU |
| siRNA 2830 | 2830 | ACGGGUGGCAGUACUAAGU | 8320 | ACUUAGUACUGCCACCCGU |
| siRNA 2831 | 2831 | CGGGUGGCAGUACUAAGUA | 8321 | UACUUAGUACUGCCACCCG |
| siRNA 2832 | 2832 | GGGUGGCAGUACUAAGUAG | 8322 | CUACUUAGUACUGCCACCC |
| siRNA 2833 | 2833 | GGUGGCAGUACUAAGUAGA | 8323 | UCUACUUAGUACUGCCACC |
| siRNA 2834 | 2834 | GUGGCAGUACUAAGUAGAA | 8324 | UUCUACUUAGUACUGCCAC |
| siRNA 2835 | 2835 | UGGCAGUACUAAGUAGAAU | 8325 | AUUCUACUUAGUACUGCCA |
| siRNA 2836 | 2836 | GGCAGUACUAAGUAGAAUU | 8326 | AAUUCUACUUAGUACUGCC |
| siRNA 2837 | 2837 | GCAGUACUAAGUAGAAUUA | 8327 | UAAUUCUACUUAGUACUGC |
| siRNA 2838 | 2838 | CAGUACUAAGUAGAAUUAU | 8328 | AUAAUUCUACUUAGUACUG |
| siRNA 2839 | 2839 | AGUACUAAGUAGAAUUAUA | 8329 | UAUAAUUCUACUUAGUACU |
| siRNA 2840 | 2840 | GUACUAAGUAGAAUUAUAG | 8330 | CUAUAAUUCUACUUAGUAC |
| siRNA 2841 | 2841 | UACUAAGUAGAAUUAUAGA | 8331 | UCUAUAAUUCUACUUAGUA |
| siRNA 2842 | 2842 | ACUAAGUAGAAUUAUAGAA | 8332 | UUCUAUAAUUCUACUUAGU |
| siRNA 2843 | 2843 | CUAAGUAGAAUUAUAGAAA | 8333 | UUUCUAUAAUUCUACUUAG |
| siRNA 2844 | 2844 | UAAGUAGAAUUAUAGAAAU | 8334 | AUUUCUAUAAUUCUACUUA |
| siRNA 2845 | 2845 | AAGUAGAAUUAUAGAAAUU | 8335 | AAUUUCUAUAAUUCUACUU |
| siRNA 2846 | 2846 | AGUAGAAUUAUAGAAAUUC | 8336 | GAAUUUCUAUAAUUCUACU |
| siRNA 2847 | 2847 | GUAGAAUUAUAGAAAUUCU | 8337 | AGAAUUUCUAUAAUUCUAC |
| siRNA 2848 | 2848 | UAGAAUUAUAGAAAUUCUG | 8338 | CAGAAUUUCUAUAAUUCUA |
| siRNA 2849 | 2849 | AGAAUUAUAGAAAUUCUGC | 8339 | GCAGAAUUUCUAUAAUUCU |
| siRNA 2850 | 2850 | GAAUUAUAGAAAUUCUGCA | 8340 | UGCAGAAUUUCUAUAAUUC |
| siRNA 2851 | 2851 | AAUUAUAGAAAUUCUGCAA | 8341 | UUGCAGAAUUUCUAUAAUU |
| siRNA 2852 | 2852 | AUUAUAGAAAUUCUGCAAG | 8342 | CUUGCAGAAUUUCUAUAAU |
| siRNA 2853 | 2853 | UUAUAGAAAUUCUGCAAGU | 8343 | ACUUGCAGAAUUUCUAUAA |
| siRNA 2854 | 2854 | UAUAGAAAUUCUGCAAGUU | 8344 | AACUUGCAGAAUUUCUAUA |
| siRNA 2855 | 2855 | AUAGAAAUUCUGCAAGUUU | 8345 | AAACUUGCAGAAUUUCUAU |
| siRNA 2856 | 2856 | UAGAAAUUCUGCAAGUUUU | 8346 | AAAACUUGCAGAAUUUCUA |
| siRNA 2857 | 2857 | AGAAAUUCUGCAAGUUUUU | 8347 | AAAAACUUGCAGAAUUUCU |
| siRNA 2858 | 2858 | GAAAUUCUGCAAGUUUUUC | 8348 | GAAAAACUUGCAGAAUUUC |
| siRNA 2859 | 2859 | AAAUUCUGCAAGUUUUUCA | 8349 | UGAAAAACUUGCAGAAUUU |
| siRNA 2860 | 2860 | AAUUCUGCAAGUUUUUCAA | 8350 | UUGAAAAACUUGCAGAAUU |
| siRNA 2861 | 2861 | AUUCUGCAAGUUUUUCAAG | 8351 | CUUGAAAAACUUGCAGAAU |
| siRNA 2862 | 2862 | UUCUGCAAGUUUUUCAAGA | 8352 | UCUUGAAAAACUUGCAGAA |
| siRNA 2863 | 2863 | UCUGCAAGUUUUUCAAGAU | 8353 | AUCUUGAAAAACUUGCAGA |
| siRNA 2864 | 2864 | CUGCAAGUUUUUCAAGAUU | 8354 | AAUCUUGAAAAACUUGCAG |
| siRNA 2865 | 2865 | UGCAAGUUUUUCAAGAUUU | 8355 | AAAUCUUGAAAAACUUGCA |
| siRNA 2866 | 2866 | GCAAGUUUUUCAAGAUUUG | 8356 | CAAAUCUUGAAAAACUUGC |
| siRNA 2867 | 2867 | CAAGUUUUUCAAGAUUUGA | 8357 | UCAAAUCUUGAAAAACUUG |
| siRNA 2868 | 2868 | AAGUUUUUCAAGAUUUGAA | 8358 | UUCAAAUCUUGAAAAACUU |
| siRNA 2869 | 2869 | AGUUUUUCAAGAUUUGAAU | 8359 | AUUCAAAUCUUGAAAAACU |
| siRNA 2870 | 2870 | GUUUUUCAAGAUUUGAAUA | 8360 | UAUUCAAAUCUUGAAAAAC |
| siRNA 2871 | 2871 | UUUUUCAAGAUUUGAAUAA | 8361 | UUAUUCAAAUCUUGAAAAA |
| siRNA 2872 | 2872 | UUUUCAAGAUUUGAAUAAU | 8362 | AUUAUUCAAAUCUUGAAAA |
| siRNA 2873 | 2873 | UUUCAAGAUUUGAAUAAUU | 8363 | AAUUAUUCAAAUCUUGAAA |
| siRNA 2874 | 2874 | UUCAAGAUUUGAAUAAUUU | 8364 | AAAUUAUUCAAAUCUUGAA |
| siRNA 2875 | 2875 | UCAAGAUUUGAAUAAUUUC | 8365 | GAAAUUAUUCAAAUCUUGA |
| siRNA 2876 | 2876 | CAAGAUUUGAAUAAUUUCA | 8366 | UGAAAUUAUUCAAAUCUUG |
| siRNA 2877 | 2877 | AAGAUUUGAAUAAUUUCAA | 8367 | UUGAAAUUAUUCAAAUCUU |
| siRNA 2878 | 2878 | AGAUUUGAAUAAUUUCAAU | 8368 | AUUGAAAUUAUUCAAAUCU |
| siRNA 2879 | 2879 | GAUUUGAAUAAUUUCAAUG | 8369 | CAUUGAAAUUAUUCAAAUC |
| siRNA 2880 | 2880 | AUUUGAAUAAUUUCAAUGG | 8370 | CCAUUGAAAUUAUUCAAAU |
| siRNA 2881 | 2881 | UUUGAAUAAUUUCAAUGGC | 8371 | GCCAUUGAAAUUAUUCAAA |
| siRNA 2882 | 2882 | UUGAAUAAUUUCAAUGGCG | 8372 | CGCCAUUGAAAUUAUUCAA |
| siRNA 2883 | 2883 | UGAAUAAUUUCAAUGGCGU | 8373 | ACGCCAUUGAAAUUAUUCA |
| siRNA 2884 | 2884 | GAAUAAUUUCAAUGGCGUA | 8374 | UACGCCAUUGAAAUUAUUC |
| siRNA 2885 | 2885 | AAUAAUUUCAAUGGCGUAU | 8375 | AUACGCCAUUGAAAUUAUU |
| siRNA 2886 | 2886 | AUAAUUUCAAUGGCGUAUU | 8376 | AAUACGCCAUUGAAAUUAU |
| siRNA 2887 | 2887 | UAAUUUCAAUGGCGUAUUG | 8377 | CAAUACGCCAUUGAAAUUA |
| siRNA 2888 | 2888 | AAUUUCAAUGGCGUAUUGG | 8378 | CCAAUACGCCAUUGAAAUU |
| siRNA 2889 | 2889 | AUUUCAAUGGCGUAUUGGA | 8379 | UCCAAUACGCCAUUGAAAU |
| siRNA 2890 | 2890 | UUUCAAUGGCGUAUUGGAG | 8380 | CUCCAAUACGCCAUUGAAA |
| siRNA 2891 | 2891 | UUCAAUGGCGUAUUGGAGA | 8381 | UCUCCAAUACGCCAUUGAA |
| siRNA 2892 | 2892 | UCAAUGGCGUAUUGGAGAU | 8382 | AUCUCCAAUACGCCAUUGA |
| siRNA 2893 | 2893 | CAAUGGCGUAUUGGAGAUA | 8383 | UAUCUCCAAUACGCCAUUG |
| siRNA 2894 | 2894 | AAUGGCGUAUUGGAGAUAG | 8384 | CUAUCUCCAAUACGCCAUU |
| siRNA 2895 | 2895 | AUGGCGUAUUGGAGAUAGU | 8385 | ACUAUCUCCAAUACGCCAU |
| siRNA 2896 | 2896 | UGGCGUAUUGGAGAUAGUC | 8386 | GACUAUCUCCAAUACGCCA |
| siRNA 2897 | 2897 | GGCGUAUUGGAGAUAGUCA | 8387 | UGACUAUCUCCAAUACGCC |
| siRNA 2898 | 2898 | GCGUAUUGGAGAUAGUCAG | 8388 | CUGACUAUCUCCAAUACGC |
| siRNA 2899 | 2899 | CGUAUUGGAGAUAGUCAGU | 8389 | ACUGACUAUCUCCAAUACG |
| siRNA 2900 | 2900 | GUAUUGGAGAUAGUCAGUG | 8390 | CACUGACUAUCUCCAAUAC |
| siRNA 2901 | 2901 | UAUUGGAGAUAGUCAGUGC | 8391 | GCACUGACUAUCUCCAAUA |
| siRNA 2902 | 2902 | AUUGGAGAUAGUCAGUGCA | 8392 | UGCACUGACUAUCUCCAAU |
| siRNA 2903 | 2903 | UUGGAGAUAGUCAGUGCAG | 8393 | CUGCACUGACUAUCUCCAA |
| siRNA 2904 | 2904 | UGGAGAUAGUCAGUGCAGU | 8394 | ACUGCACUGACUAUCUCCA |
| siRNA 2905 | 2905 | GGAGAUAGUCAGUGCAGUA | 8395 | UACUGCACUGACUAUCUCC |
| siRNA 2906 | 2906 | GAGAUAGUCAGUGCAGUAA | 8396 | UUACUGCACUGACUAUCUC |
| siRNA 2907 | 2907 | AGAUAGUCAGUGCAGUAAA | 8397 | UUUACUGCACUGACUAUCU |
| siRNA 2908 | 2908 | GAUAGUCAGUGCAGUAAAU | 8398 | AUUUACUGCACUGACUAUC |
| siRNA 2909 | 2909 | AUAGUCAGUGCAGUAAAUU | 8399 | AAUUUACUGCACUGACUAU |
| siRNA 2910 | 2910 | UAGUCAGUGCAGUAAAUUC | 8400 | GAAUUUACUGCACUGACUA |
| siRNA 2911 | 2911 | AGUCAGUGCAGUAAAUUCA | 8401 | UGAAUUUACUGCACUGACU |
| siRNA 2912 | 2912 | GUCAGUGCAGUAAAUUCAG | 8402 | CUGAAUUUACUGCACUGAC |
| siRNA 2913 | 2913 | UCAGUGCAGUAAAUUCAGU | 8403 | ACUGAAUUUACUGCACUGA |
| siRNA 2914 | 2914 | CAGUGCAGUAAAUUCAGUG | 8404 | CACUGAAUUUACUGCACUG |
| siRNA 2915 | 2915 | AGUGCAGUAAAUUCAGUGU | 8405 | ACACUGAAUUUACUGCACU |
| siRNA 2916 | 2916 | GUGCAGUAAAUUCAGUGUC | 8406 | GACACUGAAUUUACUGCAC |
| siRNA 2917 | 2917 | UGCAGUAAAUUCAGUGUCA | 8407 | UGACACUGAAUUUACUGCA |
| siRNA 2918 | 2918 | GCAGUAAAUUCAGUGUCAG | 8408 | CUGACACUGAAUUUACUGC |
| siRNA 2919 | 2919 | CAGUAAAUUCAGUGUCAGU | 8409 | ACUGACACUGAAUUUACUG |
| siRNA 2920 | 2920 | AGUAAAUUCAGUGUCAGUA | 8410 | UACUGACACUGAAUUUACU |
| siRNA 2921 | 2921 | GUAAAUUCAGUGUCAGUAU | 8411 | AUACUGACACUGAAUUUAC |
| siRNA 2922 | 2922 | UAAAUUCAGUGUCAGUAUA | 8412 | UAUACUGACACUGAAUUUA |
| siRNA 2923 | 2923 | AAAUUCAGUGUCAGUAUAC | 8413 | GUAUACUGACACUGAAUUU |
| siRNA 2924 | 2924 | AAUUCAGUGUCAGUAUACA | 8414 | UGUAUACUGACACUGAAUU |
| siRNA 2925 | 2925 | AUUCAGUGUCAGUAUACAG | 8415 | CUGUAUACUGACACUGAAU |
| siRNA 2926 | 2926 | UUCAGUGUCAGUAUACAGA | 8416 | UCUGUAUACUGACACUGAA |
| siRNA 2927 | 2927 | UCAGUGUCAGUAUACAGAC | 8417 | GUCUGUAUACUGACACUGA |
| siRNA 2928 | 2928 | CAGUGUCAGUAUACAGACU | 8418 | AGUCUGUAUACUGACACUG |
| siRNA 2929 | 2929 | AGUGUCAGUAUACAGACUA | 8419 | UAGUCUGUAUACUGACACU |
| siRNA 2930 | 2930 | GUGUCAGUAUACAGACUAG | 8420 | CUAGUCUGUAUACUGACAC |
| siRNA 2931 | 2931 | UGUCAGUAUACAGACUAGA | 8421 | UCUAGUCUGUAUACUGACA |
| siRNA 2932 | 2932 | GUCAGUAUACAGACUAGAC | 8422 | GUCUAGUCUGUAUACUGAC |
| siRNA 2933 | 2933 | UCAGUAUACAGACUAGACC | 8423 | GGUCUAGUCUGUAUACUGA |
| siRNA 2934 | 2934 | CAGUAUACAGACUAGACCA | 8424 | UGGUCUAGUCUGUAUACUG |
| siRNA 2935 | 2935 | AGUAUACAGACUAGACCAU | 8425 | AUGGUCUAGUCUGUAUACU |
| siRNA 2936 | 2936 | GUAUACAGACUAGACCAUA | 8426 | UAUGGUCUAGUCUGUAUAC |
| siRNA 2937 | 2937 | UAUACAGACUAGACCAUAC | 8427 | GUAUGGUCUAGUCUGUAUA |
| siRNA 2938 | 2938 | AUACAGACUAGACCAUACC | 8428 | GGUAUGGUCUAGUCUGUAU |
| siRNA 2939 | 2939 | UACAGACUAGACCAUACCU | 8429 | AGGUAUGGUCUAGUCUGUA |
| siRNA 2940 | 2940 | ACAGACUAGACCAUACCUU | 8430 | AAGGUAUGGUCUAGUCUGU |
| siRNA 2941 | 2941 | CAGACUAGACCAUACCUUU | 8431 | AAAGGUAUGGUCUAGUCUG |
| siRNA 2942 | 2942 | AGACUAGACCAUACCUUUG | 8432 | CAAAGGUAUGGUCUAGUCU |
| siRNA 2943 | 2943 | GACUAGACCAUACCUUUGA | 8433 | UCAAAGGUAUGGUCUAGUC |
| siRNA 2944 | 2944 | ACUAGACCAUACCUUUGAG | 8434 | CUCAAAGGUAUGGUCUAGU |
| siRNA 2945 | 2945 | CUAGACCAUACCUUUGAGG | 8435 | CCUCAAAGGUAUGGUCUAG |
| siRNA 2946 | 2946 | UAGACCAUACCUUUGAGGC | 8436 | GCCUCAAAGGUAUGGUCUA |
| siRNA 2947 | 2947 | AGACCAUACCUUUGAGGCA | 8437 | UGCCUCAAAGGUAUGGUCU |
| siRNA 2948 | 2948 | GACCAUACCUUUGAGGCAC | 8438 | GUGCCUCAAAGGUAUGGUC |
| siRNA 2949 | 2949 | ACCAUACCUUUGAGGCACU | 8439 | AGUGCCUCAAAGGUAUGGU |
| siRNA 2950 | 2950 | CCAUACCUUUGAGGCACUG | 8440 | CAGUGCCUCAAAGGUAUGG |
| siRNA 2951 | 2951 | CAUACCUUUGAGGCACUGC | 8441 | GCAGUGCCUCAAAGGUAUG |
| siRNA 2952 | 2952 | AUACCUUUGAGGCACUGCA | 8442 | UGCAGUGCCUCAAAGGUAU |
| siRNA 2953 | 2953 | UACCUUUGAGGCACUGCAG | 8443 | CUGCAGUGCCUCAAAGGUA |
| siRNA 2954 | 2954 | ACCUUUGAGGCACUGCAGG | 8444 | CCUGCAGUGCCUCAAAGGU |
| siRNA 2955 | 2955 | CCUUUGAGGCACUGCAGGA | 8445 | UCCUGCAGUGCCUCAAAGG |
| siRNA 2956 | 2956 | CUUUGAGGCACUGCAGGAA | 8446 | UUCCUGCAGUGCCUCAAAG |
| siRNA 2957 | 2957 | UUUGAGGCACUGCAGGAAA | 8447 | UUUCCUGCAGUGCCUCAAA |
| siRNA 2958 | 2958 | UUGAGGCACUGCAGGAAAG | 8448 | CUUUCCUGCAGUGCCUCAA |
| siRNA 2959 | 2959 | UGAGGCACUGCAGGAAAGG | 8449 | CCUUUCCUGCAGUGCCUCA |
| siRNA 2960 | 2960 | GAGGCACUGCAGGAAAGGA | 8450 | UCCUUUCCUGCAGUGCCUC |
| siRNA 2961 | 2961 | AGGCACUGCAGGAAAGGAA | 8451 | UUCCUUUCCUGCAGUGCCU |
| siRNA 2962 | 2962 | GGCACUGCAGGAAAGGAAA | 8452 | UUUCCUUUCCUGCAGUGCC |
| siRNA 2963 | 2963 | GCACUGCAGGAAAGGAAAA | 8453 | UUUUCCUUUCCUGCAGUGC |
| siRNA 2964 | 2964 | CACUGCAGGAAAGGAAAAG | 8454 | CUUUUCCUUUCCUGCAGUG |
| siRNA 2965 | 2965 | ACUGCAGGAAAGGAAAAGG | 8455 | CCUUUUCCUUUCCUGCAGU |
| siRNA 2966 | 2966 | CUGCAGGAAAGGAAAAGGA | 8456 | UCCUUUUCCUUUCCUGCAG |
| siRNA 2967 | 2967 | UGCAGGAAAGGAAAAGGAA | 8457 | UUCCUUUUCCUUUCCUGCA |
| siRNA 2968 | 2968 | GCAGGAAAGGAAAAGGAAA | 8458 | UUUCCUUUUCCUUUCCUGC |
| siRNA 2969 | 2969 | CAGGAAAGGAAAAGGAAAA | 8459 | UUUUCCUUUUCCUUUCCUG |
| siRNA 2970 | 2970 | AGGAAAGGAAAAGGAAAAU | 8460 | AUUUUCCUUUUCCUUUCCU |
| siRNA 2971 | 2971 | GGAAAGGAAAAGGAAAAUU | 8461 | AAUUUUCCUUUUCCUUUCC |
| siRNA 2972 | 2972 | GAAAGGAAAAGGAAAAUUU | 8462 | AAAUUUUCCUUUUCCUUUC |
| siRNA 2973 | 2973 | AAAGGAAAAGGAAAAUUUU | 8463 | AAAAUUUUCCUUUUCCUUU |
| siRNA 2974 | 2974 | AAGGAAAAGGAAAAUUUUG | 8464 | CAAAAUUUUCCUUUUCCUU |
| siRNA 2975 | 2975 | AGGAAAAGGAAAAUUUUGG | 8465 | CCAAAAUUUUCCUUUUCCU |
| siRNA 2976 | 2976 | GGAAAAGGAAAAUUUUGGA | 8466 | UCCAAAAUUUUCCUUUUCC |
| siRNA 2977 | 2977 | GAAAAGGAAAAUUUUGGAC | 8467 | GUCCAAAAUUUUCCUUUUC |
| siRNA 2978 | 2978 | AAAAGGAAAAUUUUGGACG | 8468 | CGUCCAAAAUUUUCCUUUU |
| siRNA 2979 | 2979 | AAAGGAAAAUUUUGGACGA | 8469 | UCGUCCAAAAUUUUCCUUU |
| siRNA 2980 | 2980 | AAGGAAAAUUUUGGACGAA | 8470 | UUCGUCCAAAAUUUUCCUU |
| siRNA 2981 | 2981 | AGGAAAAUUUUGGACGAAG | 8471 | CUUCGUCCAAAAUUUUCCU |
| siRNA 2982 | 2982 | GGAAAAUUUUGGACGAAGC | 8472 | GCUUCGUCCAAAAUUUUCC |
| siRNA 2983 | 2983 | GAAAAUUUUGGACGAAGCU | 8473 | AGCUUCGUCCAAAAUUUUC |
| siRNA 2984 | 2984 | AAAAUUUUGGACGAAGCUG | 8474 | CAGCUUCGUCCAAAAUUUU |
| siRNA 2985 | 2985 | AAAUUUUGGACGAAGCUGU | 8475 | ACAGCUUCGUCCAAAAUUU |
| siRNA 2986 | 2986 | AAUUUUGGACGAAGCUGUG | 8476 | CACAGCUUCGUCCAAAAUU |
| siRNA 2987 | 2987 | AUUUUGGACGAAGCUGUGG | 8477 | CCACAGCUUCGUCCAAAAU |
| siRNA 2988 | 2988 | UUUUGGACGAAGCUGUGGA | 8478 | UCCACAGCUUCGUCCAAAA |
| siRNA 2989 | 2989 | UUUGGACGAAGCUGUGGAA | 8479 | UUCCACAGCUUCGUCCAAA |
| siRNA 2990 | 2990 | UUGGACGAAGCUGUGGAAU | 8480 | AUUCCACAGCUUCGUCCAA |
| siRNA 2991 | 2991 | UGGACGAAGCUGUGGAAUU | 8481 | AAUUCCACAGCUUCGUCCA |
| siRNA 2992 | 2992 | GGACGAAGCUGUGGAAUUA | 8482 | UAAUUCCACAGCUUCGUCC |
| siRNA 2993 | 2993 | GACGAAGCUGUGGAAUUAA | 8483 | UUAAUUCCACAGCUUCGUC |
| siRNA 2994 | 2994 | ACGAAGCUGUGGAAUUAAG | 8484 | CUUAAUUCCACAGCUUCGU |
| siRNA 2995 | 2995 | CGAAGCUGUGGAAUUAAGU | 8485 | ACUUAAUUCCACAGCUUCG |
| siRNA 2996 | 2996 | GAAGCUGUGGAAUUAAGUC | 8486 | GACUUAAUUCCACAGCUUC |
| siRNA 2997 | 2997 | AAGCUGUGGAAUUAAGUCA | 8487 | UGACUUAAUUCCACAGCUU |
| siRNA 2998 | 2998 | AGCUGUGGAAUUAAGUCAA | 8488 | UUGACUUAAUUCCACAGCU |
| siRNA 2999 | 2999 | GCUGUGGAAUUAAGUCAAG | 8489 | CUUGACUUAAUUCCACAGC |
| siRNA 3000 | 3000 | CUGUGGAAUUAAGUCAAGA | 8490 | UCUUGACUUAAUUCCACAG |
| siRNA 3001 | 3001 | UGUGGAAUUAAGUCAAGAU | 8491 | AUCUUGACUUAAUUCCACA |
| siRNA 3002 | 3002 | GUGGAAUUAAGUCAAGAUC | 8492 | GAUCUUGACUUAAUUCCAC |
| siRNA 3003 | 3003 | UGGAAUUAAGUCAAGAUCA | 8493 | UGAUCUUGACUUAAUUCCA |
| siRNA 3004 | 3004 | GGAAUUAAGUCAAGAUCAC | 8494 | GUGAUCUUGACUUAAUUCC |
| siRNA 3005 | 3005 | GAAUUAAGUCAAGAUCACU | 8495 | AGUGAUCUUGACUUAAUUC |
| siRNA 3006 | 3006 | AAUUAAGUCAAGAUCACUU | 8496 | AAGUGAUCUUGACUUAAUU |
| siRNA 3007 | 3007 | AUUAAGUCAAGAUCACUUU | 8497 | AAAGUGAUCUUGACUUAAU |
| siRNA 3008 | 3008 | UUAAGUCAAGAUCACUUUA | 8498 | UAAAGUGAUCUUGACUUAA |
| siRNA 3009 | 3009 | UAAGUCAAGAUCACUUUAA | 8499 | UUAAAGUGAUCUUGACUUA |
| siRNA 3010 | 3010 | AAGUCAAGAUCACUUUAAA | 8500 | UUUAAAGUGAUCUUGACUU |
| siRNA 3011 | 3011 | AGUCAAGAUCACUUUAAAA | 8501 | UUUUAAAGUGAUCUUGACU |
| siRNA 3012 | 3012 | GUCAAGAUCACUUUAAAAA | 8502 | UUUUUAAAGUGAUCUUGAC |
| siRNA 3013 | 3013 | UCAAGAUCACUUUAAAAAA | 8503 | UUUUUUAAAGUGAUCUUGA |
| siRNA 3014 | 3014 | CAAGAUCACUUUAAAAAAU | 8504 | AUUUUUUAAAGUGAUCUUG |
| siRNA 3015 | 3015 | AAGAUCACUUUAAAAAAUA | 8505 | UAUUUUUUAAAGUGAUCUU |
| siRNA 3016 | 3016 | AGAUCACUUUAAAAAAUAC | 8506 | GUAUUUUUUAAAGUGAUCU |
| siRNA 3017 | 3017 | GAUCACUUUAAAAAAUACC | 8507 | GGUAUUUUUUAAAGUGAUC |
| siRNA 3018 | 3018 | AUCACUUUAAAAAAUACCU | 8508 | AGGUAUUUUUUAAAGUGAU |
| siRNA 3019 | 3019 | UCACUUUAAAAAAUACCUA | 8509 | UAGGUAUUUUUUAAAGUGA |
| siRNA 3020 | 3020 | CACUUUAAAAAAUACCUAG | 8510 | CUAGGUAUUUUUUAAAGUG |
| siRNA 3021 | 3021 | ACUUUAAAAAAUACCUAGU | 8511 | ACUAGGUAUUUUUUAAAGU |
| siRNA 3022 | 3022 | CUUUAAAAAAUACCUAGUA | 8512 | UACUAGGUAUUUUUUAAAG |
| siRNA 3023 | 3023 | UUUAAAAAAUACCUAGUAA | 8513 | UUACUAGGUAUUUUUUAAA |
| siRNA 3024 | 3024 | UUAAAAAAUACCUAGUAAA | 8514 | UUUACUAGGUAUUUUUUAA |
| siRNA 3025 | 3025 | UAAAAAAUACCUAGUAAAA | 8515 | UUUUACUAGGUAUUUUUUA |
| siRNA 3026 | 3026 | AAAAAAUACCUAGUAAAAC | 8516 | GUUUUACUAGGUAUUUUUU |
| siRNA 3027 | 3027 | AAAAAUACCUAGUAAAACU | 8517 | AGUUUUACUAGGUAUUUUU |
| siRNA 3028 | 3028 | AAAAUACCUAGUAAAACUU | 8518 | AAGUUUUACUAGGUAUUUU |
| siRNA 3029 | 3029 | AAAUACCUAGUAAAACUUA | 8519 | UAAGUUUUACUAGGUAUUU |
| siRNA 3030 | 3030 | AAUACCUAGUAAAACUUAA | 8520 | UUAAGUUUUACUAGGUAUU |
| siRNA 3031 | 3031 | AUACCUAGUAAAACUUAAG | 8521 | CUUAAGUUUUACUAGGUAU |
| siRNA 3032 | 3032 | UACCUAGUAAAACUUAAGU | 8522 | ACUUAAGUUUUACUAGGUA |
| siRNA 3033 | 3033 | ACCUAGUAAAACUUAAGUC | 8523 | GACUUAAGUUUUACUAGGU |
| siRNA 3034 | 3034 | CCUAGUAAAACUUAAGUCA | 8524 | UGACUUAAGUUUUACUAGG |
| siRNA 3035 | 3035 | CUAGUAAAACUUAAGUCAA | 8525 | UUGACUUAAGUUUUACUAG |
| siRNA 3036 | 3036 | UAGUAAAACUUAAGUCAAU | 8526 | AUUGACUUAAGUUUUACUA |
| siRNA 3037 | 3037 | AGUAAAACUUAAGUCAAUC | 8527 | GAUUGACUUAAGUUUUACU |
| siRNA 3038 | 3038 | GUAAAACUUAAGUCAAUCA | 8528 | UGAUUGACUUAAGUUUUAC |
| siRNA 3039 | 3039 | UAAAACUUAAGUCAAUCAA | 8529 | UUGAUUGACUUAAGUUUUA |
| siRNA 3040 | 3040 | AAAACUUAAGUCAAUCAAU | 8530 | AUUGAUUGACUUAAGUUUU |
| siRNA 3041 | 3041 | AAACUUAAGUCAAUCAAUC | 8531 | GAUUGAUUGACUUAAGUUU |
| siRNA 3042 | 3042 | AACUUAAGUCAAUCAAUCC | 8532 | GGAUUGAUUGACUUAAGUU |
| siRNA 3043 | 3043 | ACUUAAGUCAAUCAAUCCA | 8533 | UGGAUUGAUUGACUUAAGU |
| siRNA 3044 | 3044 | CUUAAGUCAAUCAAUCCAC | 8534 | GUGGAUUGAUUGACUUAAG |
| siRNA 3045 | 3045 | UUAAGUCAAUCAAUCCACC | 8535 | GGUGGAUUGAUUGACUUAA |
| siRNA 3046 | 3046 | UAAGUCAAUCAAUCCACCU | 8536 | AGGUGGAUUGAUUGACUUA |
| siRNA 3047 | 3047 | AAGUCAAUCAAUCCACCUU | 8537 | AAGGUGGAUUGAUUGACUU |
| siRNA 3048 | 3048 | AGUCAAUCAAUCCACCUUG | 8538 | CAAGGUGGAUUGAUUGACU |
| siRNA 3049 | 3049 | GUCAAUCAAUCCACCUUGU | 8539 | ACAAGGUGGAUUGAUUGAC |
| siRNA 3050 | 3050 | UCAAUCAAUCCACCUUGUG | 8540 | CACAAGGUGGAUUGAUUGA |
| siRNA 3051 | 3051 | CAAUCAAUCCACCUUGUGU | 8541 | ACACAAGGUGGAUUGAUUG |
| siRNA 3052 | 3052 | AAUCAAUCCACCUUGUGUG | 8542 | CACACAAGGUGGAUUGAUU |
| siRNA 3053 | 3053 | AUCAAUCCACCUUGUGUGC | 8543 | GCACACAAGGUGGAUUGAU |
| siRNA 3054 | 3054 | UCAAUCCACCUUGUGUGCC | 8544 | GGCACACAAGGUGGAUUGA |
| siRNA 3055 | 3055 | CAAUCCACCUUGUGUGCCU | 8545 | AGGCACACAAGGUGGAUUG |
| siRNA 3056 | 3056 | AAUCCACCUUGUGUGCCUU | 8546 | AAGGCACACAAGGUGGAUU |
| siRNA 3057 | 3057 | AUCCACCUUGUGUGCCUUU | 8547 | AAAGGCACACAAGGUGGAU |
| siRNA 3058 | 3058 | UCCACCUUGUGUGCCUUUU | 8548 | AAAAGGCACACAAGGUGGA |
| siRNA 3059 | 3059 | CCACCUUGUGUGCCUUUUU | 8549 | AAAAAGGCACACAAGGUGG |
| siRNA 3060 | 3060 | CACCUUGUGUGCCUUUUUU | 8550 | AAAAAAGGCACACAAGGUG |
| siRNA 3061 | 3061 | ACCUUGUGUGCCUUUUUUU | 8551 | AAAAAAAGGCACACAAGGU |
| siRNA 3062 | 3062 | CCUUGUGUGCCUUUUUUUG | 8552 | CAAAAAAAGGCACACAAGG |
| siRNA 3063 | 3063 | CUUGUGUGCCUUUUUUUGG | 8553 | CCAAAAAAAGGCACACAAG |
| siRNA 3064 | 3064 | UUGUGUGCCUUUUUUUGGA | 8554 | UCCAAAAAAAGGCACACAA |
| siRNA 3065 | 3065 | UGUGUGCCUUUUUUUGGAA | 8555 | UUCCAAAAAAAGGCACACA |
| siRNA 3066 | 3066 | GUGUGCCUUUUUUUGGAAU | 8556 | AUUCCAAAAAAAGGCACAC |
| siRNA 3067 | 3067 | UGUGCCUUUUUUUGGAAUA | 8557 | UAUUCCAAAAAAAGGCACA |
| siRNA 3068 | 3068 | GUGCCUUUUUUUGGAAUAU | 8558 | AUAUUCCAAAAAAAGGCAC |
| siRNA 3069 | 3069 | UGCCUUUUUUUGGAAUAUA | 8559 | UAUAUUCCAAAAAAAGGCA |
| siRNA 3070 | 3070 | GCCUUUUUUUGGAAUAUAU | 8560 | AUAUAUUCCAAAAAAAGGC |
| siRNA 3071 | 3071 | CCUUUUUUUGGAAUAUAUU | 8561 | AAUAUAUUCCAAAAAAAGG |
| siRNA 3072 | 3072 | CUUUUUUUGGAAUAUAUUU | 8562 | AAAUAUAUUCCAAAAAAAG |
| siRNA 3073 | 3073 | UUUUUUUGGAAUAUAUUUA | 8563 | UAAAUAUAUUCCAAAAAAA |
| siRNA 3074 | 3074 | UUUUUUGGAAUAUAUUUAA | 8564 | UUAAAUAUAUUCCAAAAAA |
| siRNA 3075 | 3075 | UUUUUGGAAUAUAUUUAAC | 8565 | GUUAAAUAUAUUCCAAAAA |
| siRNA 3076 | 3076 | UUUUGGAAUAUAUUUAACA | 8566 | UGUUAAAUAUAUUCCAAAA |
| siRNA 3077 | 3077 | UUUGGAAUAUAUUUAACAA | 8567 | UUGUUAAAUAUAUUCCAAA |
| siRNA 3078 | 3078 | UUGGAAUAUAUUUAACAAA | 8568 | UUUGUUAAAUAUAUUCCAA |
| siRNA 3079 | 3079 | UGGAAUAUAUUUAACAAAU | 8569 | AUUUGUUAAAUAUAUUCCA |
| siRNA 3080 | 3080 | GGAAUAUAUUUAACAAAUA | 8570 | UAUUUGUUAAAUAUAUUCC |
| siRNA 3081 | 3081 | GAAUAUAUUUAACAAAUAU | 8571 | AUAUUUGUUAAAUAUAUUC |
| siRNA 3082 | 3082 | AAUAUAUUUAACAAAUAUU | 8572 | AAUAUUUGUUAAAUAUAUU |
| siRNA 3083 | 3083 | AUAUAUUUAACAAAUAUUC | 8573 | GAAUAUUUGUUAAAUAUAU |
| siRNA 3084 | 3084 | UAUAUUUAACAAAUAUUCU | 8574 | AGAAUAUUUGUUAAAUAUA |
| siRNA 3085 | 3085 | AUAUUUAACAAAUAUUCUG | 8575 | CAGAAUAUUUGUUAAAUAU |
| siRNA 3086 | 3086 | UAUUUAACAAAUAUUCUGA | 8576 | UCAGAAUAUUUGUUAAAUA |
| siRNA 3087 | 3087 | AUUUAACAAAUAUUCUGAA | 8577 | UUCAGAAUAUUUGUUAAAU |
| siRNA 3088 | 3088 | UUUAACAAAUAUUCUGAAG | 8578 | CUUCAGAAUAUUUGUUAAA |
| siRNA 3089 | 3089 | UUAACAAAUAUUCUGAAGA | 8579 | UCUUCAGAAUAUUUGUUAA |
| siRNA 3090 | 3090 | UAACAAAUAUUCUGAAGAC | 8580 | GUCUUCAGAAUAUUUGUUA |
| siRNA 3091 | 3091 | AACAAAUAUUCUGAAGACC | 8581 | GGUCUUCAGAAUAUUUGUU |
| siRNA 3092 | 3092 | ACAAAUAUUCUGAAGACCG | 8582 | CGGUCUUCAGAAUAUUUGU |
| siRNA 3093 | 3093 | CAAAUAUUCUGAAGACCGA | 8583 | UCGGUCUUCAGAAUAUUUG |
| siRNA 3094 | 3094 | AAAUAUUCUGAAGACCGAA | 8584 | UUCGGUCUUCAGAAUAUUU |
| siRNA 3095 | 3095 | AAUAUUCUGAAGACCGAAG | 8585 | CUUCGGUCUUCAGAAUAUU |
| siRNA 3096 | 3096 | AUAUUCUGAAGACCGAAGA | 8586 | UCUUCGGUCUUCAGAAUAU |
| siRNA 3097 | 3097 | UAUUCUGAAGACCGAAGAA | 8587 | UUCUUCGGUCUUCAGAAUA |
| siRNA 3098 | 3098 | AUUCUGAAGACCGAAGAAG | 8588 | CUUCUUCGGUCUUCAGAAU |
| siRNA 3099 | 3099 | UUCUGAAGACCGAAGAAGG | 8589 | CCUUCUUCGGUCUUCAGAA |
| siRNA 3100 | 3100 | UCUGAAGACCGAAGAAGGG | 8590 | CCCUUCUUCGGUCUUCAGA |
| siRNA 3101 | 3101 | CUGAAGACCGAAGAAGGGA | 8591 | UCCCUUCUUCGGUCUUCAG |
| siRNA 3102 | 3102 | UGAAGACCGAAGAAGGGAA | 8592 | UUCCCUUCUUCGGUCUUCA |
| siRNA 3103 | 3103 | GAAGACCGAAGAAGGGAAU | 8593 | AUUCCCUUCUUCGGUCUUC |
| siRNA 3104 | 3104 | AAGACCGAAGAAGGGAAUA | 8594 | UAUUCCCUUCUUCGGUCUU |
| siRNA 3105 | 3105 | AGACCGAAGAAGGGAAUAA | 8595 | UUAUUCCCUUCUUCGGUCU |
| siRNA 3106 | 3106 | GACCGAAGAAGGGAAUAAU | 8596 | AUUAUUCCCUUCUUCGGUC |
| siRNA 3107 | 3107 | ACCGAAGAAGGGAAUAAUG | 8597 | CAUUAUUCCCUUCUUCGGU |
| siRNA 3108 | 3108 | CCGAAGAAGGGAAUAAUGA | 8598 | UCAUUAUUCCCUUCUUCGG |
| siRNA 3109 | 3109 | CGAAGAAGGGAAUAAUGAU | 8599 | AUCAUUAUUCCCUUCUUCG |
| siRNA 3110 | 3110 | GAAGAAGGGAAUAAUGAUU | 8600 | AAUCAUUAUUCCCUUCUUC |
| siRNA 3111 | 3111 | AAGAAGGGAAUAAUGAUUU | 8601 | AAAUCAUUAUUCCCUUCUU |
| siRNA 3112 | 3112 | AGAAGGGAAUAAUGAUUUU | 8602 | AAAAUCAUUAUUCCCUUCU |
| siRNA 3113 | 3113 | GAAGGGAAUAAUGAUUUUU | 8603 | AAAAAUCAUUAUUCCCUUC |
| siRNA 3114 | 3114 | AAGGGAAUAAUGAUUUUUU | 8604 | AAAAAAUCAUUAUUCCCUU |
| siRNA 3115 | 3115 | AGGGAAUAAUGAUUUUUUA | 8605 | UAAAAAAUCAUUAUUCCCU |
| siRNA 3116 | 3116 | GGGAAUAAUGAUUUUUUAA | 8606 | UUAAAAAAUCAUUAUUCCC |
| siRNA 3117 | 3117 | GGAAUAAUGAUUUUUUAAA | 8607 | UUUAAAAAAUCAUUAUUCC |
| siRNA 3118 | 3118 | GAAUAAUGAUUUUUUAAAA | 8608 | UUUUAAAAAAUCAUUAUUC |
| siRNA 3119 | 3119 | AAUAAUGAUUUUUUAAAAA | 8609 | UUUUUAAAAAAUCAUUAUU |
| siRNA 3120 | 3120 | AUAAUGAUUUUUUAAAAAA | 8610 | UUUUUUAAAAAAUCAUUAU |
| siRNA 3121 | 3121 | UAAUGAUUUUUUAAAAAAG | 8611 | CUUUUUUAAAAAAUCAUUA |
| siRNA 3122 | 3122 | AAUGAUUUUUUAAAAAAGA | 8612 | UCUUUUUUAAAAAAUCAUU |
| siRNA 3123 | 3123 | AUGAUUUUUUAAAAAAGAA | 8613 | UUCUUUUUUAAAAAAUCAU |
| siRNA 3124 | 3124 | UGAUUUUUUAAAAAAGAAA | 8614 | UUUCUUUUUUAAAAAAUCA |
| siRNA 3125 | 3125 | GAUUUUUUAAAAAAGAAAG | 8615 | CUUUCUUUUUUAAAAAAUC |
| siRNA 3126 | 3126 | AUUUUUUAAAAAAGAAAGG | 8616 | CCUUUCUUUUUUAAAAAAU |
| siRNA 3127 | 3127 | UUUUUUAAAAAAGAAAGGG | 8617 | CCCUUUCUUUUUUAAAAAA |
| siRNA 3128 | 3128 | UUUUUAAAAAAGAAAGGGA | 8618 | UCCCUUUCUUUUUUAAAAA |
| siRNA 3129 | 3129 | UUUUAAAAAAGAAAGGGAA | 8619 | UUCCCUUUCUUUUUUAAAA |
| siRNA 3130 | 3130 | UUUAAAAAAGAAAGGGAAA | 8620 | UUUCCCUUUCUUUUUUAAA |
| siRNA 3131 | 3131 | UUAAAAAAGAAAGGGAAAG | 8621 | CUUUCCCUUUCUUUUUUAA |
| siRNA 3132 | 3132 | UAAAAAAGAAAGGGAAAGA | 8622 | UCUUUCCCUUUUUUUUUA |
| siRNA 3133 | 3133 | AAAAAAGAAAGGGAAAGAU | 8623 | AUCUUUCCCUUUCUUUUUU |
| siRNA 3134 | 3134 | AAAAAGAAAGGGAAAGAUU | 8624 | AAUCUUUCCCUUUCUUUUU |
| siRNA 3135 | 3135 | AAAAGAAAGGGAAAGAUUU | 8625 | AAAUCUUUCCCUUUCUUUU |
| siRNA 3136 | 3136 | AAAGAAAGGGAAAGAUUUA | 8626 | UAAAUCUUUCCCUUUCUUU |
| siRNA 3137 | 3137 | AAGAAAGGGAAAGAUUUAA | 8627 | UUAAAUCUUUCCCUUUCUU |
| siRNA 3138 | 3138 | AGAAAGGGAAAGAUUUAAU | 8628 | AUUAAAUCUUUCCCUUUCU |
| siRNA 3139 | 3139 | GAAAGGGAAAGAUUUAAUC | 8629 | GAUUAAAUCUUUCCCUUUC |
| siRNA 3140 | 3140 | AAAGGGAAAGAUUUAAUCA | 8630 | UGAUUAAAUCUUUCCCUUU |
| siRNA 3141 | 3141 | AAGGGAAAGAUUUAAUCAA | 8631 | UUGAUUAAAUCUUUCCCUU |
| siRNA 3142 | 3142 | AGGGAAAGAUUUAAUCAAU | 8632 | AUUGAUUAAAUCUUUCCCU |
| siRNA 3143 | 3143 | GGGAAAGAUUUAAUCAAUU | 8633 | AAUUGAUUAAAUCUUUCCC |
| siRNA 3144 | 3144 | GGAAAGAUUUAAUCAAUUU | 8634 | AAAUUGAUUAAAUCUUUCC |
| siRNA 3145 | 3145 | GAAAGAUUUAAUCAAUUUC | 8635 | GAAAUUGAUUAAAUCUUUC |
| siRNA 3146 | 3146 | AAAGAUUUAAUCAAUUUCA | 8636 | UGAAAUUGAUUAAAUCUUU |
| siRNA 3147 | 3147 | AAGAUUUAAUCAAUUUCAG | 8637 | CUGAAAUUGAUUAAAUCUU |
| siRNA 3148 | 3148 | AGAUUUAAUCAAUUUCAGU | 8638 | ACUGAAAUUGAUUAAAUCU |
| siRNA 3149 | 3149 | GAUUUAAUCAAUUUCAGUA | 8639 | UACUGAAAUUGAUUAAAUC |
| siRNA 3150 | 3150 | AUUUAAUCAAUUUCAGUAA | 8640 | UUACUGAAAUUGAUUAAAU |
| siRNA 3151 | 3151 | UUUAAUCAAUUUCAGUAAG | 8641 | CUUACUGAAAUUGAUUAAA |
| siRNA 3152 | 3152 | UUAAUCAAUUUCAGUAAGA | 8642 | UCUUACUGAAAUUGAUUAA |
| siRNA 3153 | 3153 | UAAUCAAUUUCAGUAAGAG | 8643 | CUCUUACUGAAAUUGAUUA |
| siRNA 3154 | 3154 | AAUCAAUUUCAGUAAGAGG | 8644 | CCUCUUACUGAAAUUGAUU |
| siRNA 3155 | 3155 | AUCAAUUUCAGUAAGAGGA | 8645 | UCCUCUUACUGAAAUUGAU |
| siRNA 3156 | 3156 | UCAAUUUCAGUAAGAGGAG | 8646 | CUCCUCUUACUGAAAUUGA |
| siRNA 3157 | 3157 | CAAUUUCAGUAAGAGGAGG | 8647 | CCUCCUCUUACUGAAAUUG |
| siRNA 3158 | 3158 | AAUUUCAGUAAGAGGAGGA | 8648 | UCCUCCUCUUACUGAAAUU |
| siRNA 3159 | 3159 | AUUUCAGUAAGAGGAGGAA | 8649 | UUCCUCCUCUUACUGAAAU |
| siRNA 3160 | 3160 | UUUCAGUAAGAGGAGGAAA | 8650 | UUUCCUCCUCUUACUGAAA |
| siRNA 3161 | 3161 | UUCAGUAAGAGGAGGAAAG | 8651 | CUUUCCUCCUCUUACUGAA |
| siRNA 3162 | 3162 | UCAGUAAGAGGAGGAAAGU | 8652 | ACUUUCCUCCUCUUACUGA |
| siRNA 3163 | 3163 | CAGUAAGAGGAGGAAAGUA | 8653 | UACUUUCCUCCUCUUACUG |
| siRNA 3164 | 3164 | AGUAAGAGGAGGAAAGUAG | 8654 | CUACUUUCCUCCUCUUACU |
| siRNA 3165 | 3165 | GUAAGAGGAGGAAAGUAGC | 8655 | GCUACUUUCCUCCUCUUAC |
| siRNA 3166 | 3166 | UAAGAGGAGGAAAGUAGCU | 8656 | AGCUACUUUCCUCCUCUUA |
| siRNA 3167 | 3167 | AAGAGGAGGAAAGUAGCUG | 8657 | CAGCUACUUUCCUCCUCUU |
| siRNA 3168 | 3168 | AGAGGAGGAAAGUAGCUGA | 8658 | UCAGCUACUUUCCUCCUCU |
| siRNA 3169 | 3169 | GAGGAGGAAAGUAGCUGAA | 8659 | UUCAGCUACUUUCCUCCUC |
| siRNA 3170 | 3170 | AGGAGGAAAGUAGCUGAAA | 8660 | UUUCAGCUACUUUCCUCCU |
| siRNA 3171 | 3171 | GGAGGAAAGUAGCUGAAAU | 8661 | AUUUCAGCUACUUUCCUCC |
| siRNA 3172 | 3172 | GAGGAAAGUAGCUGAAAUU | 8662 | AAUUUCAGCUACUUUCCUC |
| siRNA 3173 | 3173 | AGGAAAGUAGCUGAAAUUA | 8663 | UAAUUUCAGCUACUUUCCU |
| siRNA 3174 | 3174 | GGAAAGUAGCUGAAAUUAC | 8664 | GUAAUUUCAGCUACUUUCC |
| siRNA 3175 | 3175 | GAAAGUAGCUGAAAUUACU | 8665 | AGUAAUUUCAGCUACUUUC |
| siRNA 3176 | 3176 | AAAGUAGCUGAAAUUACUG | 8666 | CAGUAAUUUCAGCUACUUU |
| siRNA 3177 | 3177 | AAGUAGCUGAAAUUACUGG | 8667 | CCAGUAAUUUCAGCUACUU |
| siRNA 3178 | 3178 | AGUAGCUGAAAUUACUGGA | 8668 | UCCAGUAAUUUCAGCUACU |
| siRNA 3179 | 3179 | GUAGCUGAAAUUACUGGAG | 8669 | CUCCAGUAAUUUCAGCUAC |
| siRNA 3180 | 3180 | UAGCUGAAAUUACUGGAGA | 8670 | UCUCCAGUAAUUUCAGCUA |
| siRNA 3181 | 3181 | AGCUGAAAUUACUGGAGAA | 8671 | UUCUCCAGUAAUUUCAGCU |
| siRNA 3182 | 3182 | GCUGAAAUUACUGGAGAAA | 8672 | UUUCUCCAGUAAUUUCAGC |
| siRNA 3183 | 3183 | CUGAAAUUACUGGAGAAAU | 8673 | AUUUCUCCAGUAAUUUCAG |
| siRNA 3184 | 3184 | UGAAAUUACUGGAGAAAUU | 8674 | AAUUUCUCCAGUAAUUUCA |
| siRNA 3185 | 3185 | GAAAUUACUGGAGAAAUUC | 8675 | GAAUUUCUCCAGUAAUUUC |
| siRNA 3186 | 3186 | AAAUUACUGGAGAAAUUCA | 8676 | UGAAUUUCUCCAGUAAUUU |
| siRNA 3187 | 3187 | AAUUACUGGAGAAAUUCAG | 8677 | CUGAAUUUCUCCAGUAAUU |
| siRNA 3188 | 3188 | AUUACUGGAGAAAUUCAGC | 8678 | GCUGAAUUUCUCCAGUAAU |
| siRNA 3189 | 3189 | UUACUGGAGAAAUUCAGCA | 8679 | UGCUGAAUUUCUCCAGUAA |
| siRNA 3190 | 3190 | UACUGGAGAAAUUCAGCAG | 8680 | CUGCUGAAUUUCUCCAGUA |
| siRNA 3191 | 3191 | ACUGGAGAAAUUCAGCAGU | 8681 | ACUGCUGAAUUUCUCCAGU |
| siRNA 3192 | 3192 | CUGGAGAAAUUCAGCAGUA | 8682 | UACUGCUGAAUUUCUCCAG |
| siRNA 3193 | 3193 | UGGAGAAAUUCAGCAGUAU | 8683 | AUACUGCUGAAUUUCUCCA |
| siRNA 3194 | 3194 | GGAGAAAUUCAGCAGUAUC | 8684 | GAUACUGCUGAAUUUCUCC |
| siRNA 3195 | 3195 | GAGAAAUUCAGCAGUAUCA | 8685 | UGAUACUGCUGAAUUUCUC |
| siRNA 3196 | 3196 | AGAAAUUCAGCAGUAUCAG | 8686 | CUGAUACUGCUGAAUUUCU |
| siRNA 3197 | 3197 | GAAAUUCAGCAGUAUCAGA | 8687 | UCUGAUACUGCUGAAUUUC |
| siRNA 3198 | 3198 | AAAUUCAGCAGUAUCAGAA | 8688 | UUCUGAUACUGCUGAAUUU |
| siRNA 3199 | 3199 | AAUUCAGCAGUAUCAGAAU | 8689 | AUUCUGAUACUGCUGAAUU |
| siRNA 3200 | 3200 | AUUCAGCAGUAUCAGAAUC | 8690 | GAUUCUGAUACUGCUGAAU |
| siRNA 3201 | 3201 | UUCAGCAGUAUCAGAAUCA | 8691 | UGAUUCUGAUACUGCUGAA |
| siRNA 3202 | 3202 | UCAGCAGUAUCAGAAUCAG | 8692 | CUGAUUCUGAUACUGCUGA |
| siRNA 3203 | 3203 | CAGCAGUAUCAGAAUCAGC | 8693 | GCUGAUUCUGAUACUGCUG |
| siRNA 3204 | 3204 | AGCAGUAUCAGAAUCAGCC | 8694 | GGCUGAUUCUGAUACUGCU |
| siRNA 3205 | 3205 | GCAGUAUCAGAAUCAGCCU | 8695 | AGGCUGAUUCUGAUACUGC |
| siRNA 3206 | 3206 | CAGUAUCAGAAUCAGCCUU | 8696 | AAGGCUGAUUCUGAUACUG |
| siRNA 3207 | 3207 | AGUAUCAGAAUCAGCCUUA | 8697 | UAAGGCUGAUUCUGAUACU |
| siRNA 3208 | 3208 | GUAUCAGAAUCAGCCUUAC | 8698 | GUAAGGCUGAUUCUGAUAC |
| siRNA 3209 | 3209 | UAUCAGAAUCAGCCUUACU | 8699 | AGUAAGGCUGAUUCUGAUA |
| siRNA 3210 | 3210 | AUCAGAAUCAGCCUUACUG | 8700 | CAGUAAGGCUGAUUCUGAU |
| siRNA 3211 | 3211 | UCAGAAUCAGCCUUACUGU | 8701 | ACAGUAAGGCUGAUUCUGA |
| siRNA 3212 | 3212 | CAGAAUCAGCCUUACUGUU | 8702 | AACAGUAAGGCUGAUUCUG |
| siRNA 3213 | 3213 | AGAAUCAGCCUUACUGUUU | 8703 | AAACAGUAAGGCUGAUUCU |
| siRNA 3214 | 3214 | GAAUCAGCCUUACUGUUUA | 8704 | UAAACAGUAAGGCUGAUUC |
| siRNA 3215 | 3215 | AAUCAGCCUUACUGUUUAC | 8705 | GUAAACAGUAAGGCUGAUU |
| siRNA 3216 | 3216 | AUCAGCCUUACUGUUUACG | 8706 | CGUAAACAGUAAGGCUGAU |
| siRNA 3217 | 3217 | UCAGCCUUACUGUUUACGG | 8707 | CCGUAAACAGUAAGGCUGA |
| siRNA 3218 | 3218 | CAGCCUUACUGUUUACGGA | 8708 | UCCGUAAACAGUAAGGCUG |
| siRNA 3219 | 3219 | AGCCUUACUGUUUACGGAU | 8709 | AUCCGUAAACAGUAAGGCU |
| siRNA 3220 | 3220 | GCCUUACUGUUUACGGAUA | 8710 | UAUCCGUAAACAGUAAGGC |
| siRNA 3221 | 3221 | CCUUACUGUUUACGGAUAG | 8711 | CUAUCCGUAAACAGUAAGG |
| siRNA 3222 | 3222 | CUUACUGUUUACGGAUAGA | 8712 | UCUAUCCGUAAACAGUAAG |
| siRNA 3223 | 3223 | UUACUGUUUACGGAUAGAA | 8713 | UUCUAUCCGUAAACAGUAA |
| siRNA 3224 | 3224 | UACUGUUUACGGAUAGAAC | 8714 | GUUCUAUCCGUAAACAGUA |
| siRNA 3225 | 3225 | ACUGUUUACGGAUAGAACC | 8715 | GGUUCUAUCCGUAAACAGU |
| siRNA 3226 | 3226 | CUGUUUACGGAUAGAACCA | 8716 | UGGUUCUAUCCGUAAACAG |
| siRNA 3227 | 3227 | UGUUUACGGAUAGAACCAG | 8717 | CUGGUUCUAUCCGUAAACA |
| siRNA 3228 | 3228 | GUUUACGGAUAGAACCAGA | 8718 | UCUGGUUCUAUCCGUAAAC |
| siRNA 3229 | 3229 | UUUACGGAUAGAACCAGAU | 8719 | AUCUGGUUCUAUCCGUAAA |
| siRNA 3230 | 3230 | UUACGGAUAGAACCAGAUA | 8720 | UAUCUGGUUCUAUCCGUAA |
| siRNA 3231 | 3231 | UACGGAUAGAACCAGAUAU | 8721 | AUAUCUGGUUCUAUCCGUA |
| siRNA 3232 | 3232 | ACGGAUAGAACCAGAUAUG | 8722 | CAUAUCUGGUUCUAUCCGU |
| siRNA 3233 | 3233 | CGGAUAGAACCAGAUAUGA | 8723 | UCAUAUCUGGUUCUAUCCG |
| siRNA 3234 | 3234 | GGAUAGAACCAGAUAUGAG | 8724 | CUCAUAUCUGGUUCUAUCC |
| siRNA 3235 | 3235 | GAUAGAACCAGAUAUGAGG | 8725 | CCUCAUAUCUGGUUCUAUC |
| siRNA 3236 | 3236 | AUAGAACCAGAUAUGAGGA | 8726 | UCCUCAUAUCUGGUUCUAU |
| siRNA 3237 | 3237 | UAGAACCAGAUAUGAGGAG | 8727 | CUCCUCAUAUCUGGUUCUA |
| siRNA 3238 | 3238 | AGAACCAGAUAUGAGGAGA | 8728 | UCUCCUCAUAUCUGGUUCU |
| siRNA 3239 | 3239 | GAACCAGAUAUGAGGAGAU | 8729 | AUCUCCUCAUAUCUGGUUC |
| siRNA 3240 | 3240 | AACCAGAUAUGAGGAGAUU | 8730 | AAUCUCCUCAUAUCUGGUU |
| siRNA 3241 | 3241 | ACCAGAUAUGAGGAGAUUC | 8731 | GAAUCUCCUCAUAUCUGGU |
| siRNA 3242 | 3242 | CCAGAUAUGAGGAGAUUCU | 8732 | AGAAUCUCCUCAUAUCUGG |
| siRNA 3243 | 3243 | CAGAUAUGAGGAGAUUCUU | 8733 | AAGAAUCUCCUCAUAUCUG |
| siRNA 3244 | 3244 | AGAUAUGAGGAGAUUCUUU | 8734 | AAAGAAUCUCCUCAUAUCU |
| siRNA 3245 | 3245 | GAUAUGAGGAGAUUCUUUG | 8735 | CAAAGAAUCUCCUCAUAUC |
| siRNA 3246 | 3246 | AUAUGAGGAGAUUCUUUGA | 8736 | UCAAAGAAUCUCCUCAUAU |
| siRNA 3247 | 3247 | UAUGAGGAGAUUCUUUGAA | 8737 | UUCAAAGAAUCUCCUCAUA |
| siRNA 3248 | 3248 | AUGAGGAGAUUCUUUGAAA | 8738 | UUUCAAAGAAUCUCCUCAU |
| siRNA 3249 | 3249 | UGAGGAGAUUCUUUGAAAA | 8739 | UUUUCAAAGAAUCUCCUCA |
| siRNA 3250 | 3250 | GAGGAGAUUCUUUGAAAAC | 8740 | GUUUUCAAAGAAUCUCCUC |
| siRNA 3251 | 3251 | AGGAGAUUCUUUGAAAACC | 8741 | GGUUUUCAAAGAAUCUCCU |
| siRNA 3252 | 3252 | GGAGAUUCUUUGAAAACCU | 8742 | AGGUUUUCAAAGAAUCUCC |
| siRNA 3253 | 3253 | GAGAUUCUUUGAAAACCUU | 8743 | AAGGUUUUCAAAGAAUCUC |
| siRNA 3254 | 3254 | AGAUUCUUUGAAAACCUUA | 8744 | UAAGGUUUUCAAAGAAUCU |
| siRNA 3255 | 3255 | GAUUCUUUGAAAACCUUAA | 8745 | UUAAGGUUUUCAAAGAAUC |
| siRNA 3256 | 3256 | AUUCUUUGAAAACCUUAAC | 8746 | GUUAAGGUUUUCAAAGAAU |
| siRNA 3257 | 3257 | UUCUUUGAAAACCUUAACC | 8747 | GGUUAAGGUUUUCAAAGAA |
| siRNA 3258 | 3258 | UCUUUGAAAACCUUAACCC | 8748 | GGGUUAAGGUUUUCAAAGA |
| siRNA 3259 | 3259 | CUUUGAAAACCUUAACCCC | 8749 | GGGGUUAAGGUUUUCAAAG |
| siRNA 3260 | 3260 | UUUGAAAACCUUAACCCCA | 8750 | UGGGGUUAAGGUUUUCAAA |
| siRNA 3261 | 3261 | UUGAAAACCUUAACCCCAU | 8751 | AUGGGGUUAAGGUUUUCAA |
| siRNA 3262 | 3262 | UGAAAACCUUAACCCCAUG | 8752 | CAUGGGGUUAAGGUUUUCA |
| siRNA 3263 | 3263 | GAAAACCUUAACCCCAUGG | 8753 | CCAUGGGGUUAAGGUUUUC |
| siRNA 3264 | 3264 | AAAACCUUAACCCCAUGGG | 8754 | CCCAUGGGGUUAAGGUUUU |
| siRNA 3265 | 3265 | AAACCUUAACCCCAUGGGA | 8755 | UCCCAUGGGGUUAAGGUUU |
| siRNA 3266 | 3266 | AACCUUAACCCCAUGGGAA | 8756 | UUCCCAUGGGGUUAAGGUU |
| siRNA 3267 | 3267 | ACCUUAACCCCAUGGGAAG | 8757 | CUUCCCAUGGGGUUAAGGU |
| siRNA 3268 | 3268 | CCUUAACCCCAUGGGAAGU | 8758 | ACUUCCCAUGGGGUUAAGG |
| siRNA 3269 | 3269 | CUUAACCCCAUGGGAAGUG | 8759 | CACUUCCCAUGGGGUUAAG |
| siRNA 3270 | 3270 | UUAACCCCAUGGGAAGUGC | 8760 | GCACUUCCCAUGGGGUUAA |
| siRNA 3271 | 3271 | UAACCCCAUGGGAAGUGCA | 8761 | UGCACUUCCCAUGGGGUUA |
| siRNA 3272 | 3272 | AACCCCAUGGGAAGUGCAU | 8762 | AUGCACUUCCCAUGGGGUU |
| siRNA 3273 | 3273 | ACCCCAUGGGAAGUGCAUC | 8763 | GAUGCACUUCCCAUGGGGU |
| siRNA 3274 | 3274 | CCCCAUGGGAAGUGCAUCU | 8764 | AGAUGCACUUCCCAUGGGG |
| siRNA 3275 | 3275 | CCCAUGGGAAGUGCAUCUG | 8765 | CAGAUGCACUUCCCAUGGG |
| siRNA 3276 | 3276 | CCAUGGGAAGUGCAUCUGA | 8766 | UCAGAUGCACUUCCCAUGG |
| siRNA 3277 | 3277 | CAUGGGAAGUGCAUCUGAA | 8767 | UUCAGAUGCACUUCCCAUG |
| siRNA 3278 | 3278 | AUGGGAAGUGCAUCUGAAA | 8768 | UUUCAGAUGCACUUCCCAU |
| siRNA 3279 | 3279 | UGGGAAGUGCAUCUGAAAA | 8769 | UUUUCAGAUGCACUUCCCA |
| siRNA 3280 | 3280 | GGGAAGUGCAUCUGAAAAA | 8770 | UUUUUCAGAUGCACUUCCC |
| siRNA 3281 | 3281 | GGAAGUGCAUCUGAAAAAG | 8771 | CUUUUUCAGAUGCACUUCC |
| siRNA 3282 | 3282 | GAAGUGCAUCUGAAAAAGA | 8772 | UCUUUUUCAGAUGCACUUC |
| siRNA 3283 | 3283 | AAGUGCAUCUGAAAAAGAG | 8773 | CUCUUUUUCAGAUGCACUU |
| siRNA 3284 | 3284 | AGUGCAUCUGAAAAAGAGU | 8774 | ACUCUUUUUCAGAUGCACU |
| siRNA 3285 | 3285 | GUGCAUCUGAAAAAGAGUU | 8775 | AACUCUUUUUCAGAUGCAC |
| siRNA 3286 | 3286 | UGCAUCUGAAAAAGAGUUU | 8776 | AAACUCUUUUUCAGAUGCA |
| siRNA 3287 | 3287 | GCAUCUGAAAAAGAGUUUA | 8777 | UAAACUCUUUUUCAGAUGC |
| siRNA 3288 | 3288 | CAUCUGAAAAAGAGUUUAC | 8778 | GUAAACUCUUUUUCAGAUG |
| siRNA 3289 | 3289 | AUCUGAAAAAGAGUUUACA | 8779 | UGUAAACUCUUUUUCAGAU |
| siRNA 3290 | 3290 | UCUGAAAAAGAGUUUACAG | 8780 | CUGUAAACUCUUUUUCAGA |
| siRNA 3291 | 3291 | CUGAAAAAGAGUUUACAGA | 8781 | UCUGUAAACUCUUUUUCAG |
| siRNA 3292 | 3292 | UGAAAAAGAGUUUACAGAU | 8782 | AUCUGUAAACUCUUUUUCA |
| siRNA 3293 | 3293 | GAAAAAGAGUUUACAGAUU | 8783 | AAUCUGUAAACUCUUUUUC |
| siRNA 3294 | 3294 | AAAAAGAGUUUACAGAUUA | 8784 | UAAUCUGUAAACUCUUUUU |
| siRNA 3295 | 3295 | AAAAGAGUUUACAGAUUAU | 8785 | AUAAUCUGUAAACUCUUUU |
| siRNA 3296 | 3296 | AAAGAGUUUACAGAUUAUU | 8786 | AAUAAUCUGUAAACUCUUU |
| siRNA 3297 | 3297 | AAGAGUUUACAGAUUAUUU | 8787 | AAAUAAUCUGUAAACUCUU |
| siRNA 3298 | 3298 | AGAGUUUACAGAUUAUUUG | 8788 | CAAAUAAUCUGUAAACUCU |
| siRNA 3299 | 3299 | GAGUUUACAGAUUAUUUGU | 8789 | ACAAAUAAUCUGUAAACUC |
| siRNA 3300 | 3300 | AGUUUACAGAUUAUUUGUU | 8790 | AACAAAUAAUCUGUAAACU |
| siRNA 3301 | 3301 | GUUUACAGAUUAUUUGUUC | 8791 | GAACAAAUAAUCUGUAAAC |
| siRNA 3302 | 3302 | UUUACAGAUUAUUUGUUCA | 8792 | UGAACAAAUAAUCUGUAAA |
| siRNA 3303 | 3303 | UUACAGAUUAUUUGUUCAA | 8793 | UUGAACAAAUAAUCUGUAA |
| siRNA 3304 | 3304 | UACAGAUUAUUUGUUCAAC | 8794 | GUUGAACAAAUAAUCUGUA |
| siRNA 3305 | 3305 | ACAGAUUAUUUGUUCAACA | 8795 | UGUUGAACAAAUAAUCUGU |
| siRNA 3306 | 3306 | CAGAUUAUUUGUUCAACAA | 8796 | UUGUUGAACAAAUAAUCUG |
| siRNA 3307 | 3307 | AGAUUAUUUGUUCAACAAG | 8797 | CUUGUUGAACAAAUAAUCU |
| siRNA 3308 | 3308 | GAUUAUUUGUUCAACAAGU | 8798 | ACUUGUUGAACAAAUAAUC |
| siRNA 3309 | 3309 | AUUAUUUGUUCAACAAGUC | 8799 | GACUUGUUGAACAAAUAAU |
| siRNA 3310 | 3310 | UUAUUUGUUCAACAAGUCA | 8800 | UGACUUGUUGAACAAAUAA |
| siRNA 3311 | 3311 | UAUUUGUUCAACAAGUCAC | 8801 | GUGACUUGUUGAACAAAUA |
| siRNA 3312 | 3312 | AUUUGUUCAACAAGUCACU | 8802 | AGUGACUUGUUGAACAAAU |
| siRNA 3313 | 3313 | UUUGUUCAACAAGUCACUA | 8803 | UAGUGACUUGUUGAACAAA |
| siRNA 3314 | 3314 | UUGUUCAACAAGUCACUAG | 8804 | CUAGUGACUUGUUGAACAA |
| siRNA 3315 | 3315 | UGUUCAACAAGUCACUAGA | 8805 | UCUAGUGACUUGUUGAACA |
| siRNA 3316 | 3316 | GUUCAACAAGUCACUAGAA | 8806 | UUCUAGUGACUUGUUGAAC |
| siRNA 3317 | 3317 | UUCAACAAGUCACUAGAAA | 8807 | UUUCUAGUGACUUGUUGAA |
| siRNA 3318 | 3318 | UCAACAAGUCACUAGAAAU | 8808 | AUUUCUAGUGACUUGUUGA |
| siRNA 3319 | 3319 | CAACAAGUCACUAGAAAUU | 8809 | AAUUUCUAGUGACUUGUUG |
| siRNA 3320 | 3320 | AACAAGUCACUAGAAAUUG | 8810 | CAAUUUCUAGUGACUUGUU |
| siRNA 3321 | 3321 | ACAAGUCACUAGAAAUUGA | 8811 | UCAAUUUCUAGUGACUUGU |
| siRNA 3322 | 3322 | CAAGUCACUAGAAAUUGAA | 8812 | UUCAAUUUCUAGUGACUUG |
| siRNA 3323 | 3323 | AAGUCACUAGAAAUUGAAC | 8813 | GUUCAAUUUCUAGUGACUU |
| siRNA 3324 | 3324 | AGUCACUAGAAAUUGAACC | 8814 | GGUUCAAUUUCUAGUGACU |
| siRNA 3325 | 3325 | GUCACUAGAAAUUGAACCU | 8815 | AGGUUCAAUUUCUAGUGAC |
| siRNA 3326 | 3326 | UCACUAGAAAUUGAACCUC | 8816 | GAGGUUCAAUUUCUAGUGA |
| siRNA 3327 | 3327 | CACUAGAAAUUGAACCUCG | 8817 | CGAGGUUCAAUUUCUAGUG |
| siRNA 3328 | 3328 | ACUAGAAAUUGAACCUCGA | 8818 | UCGAGGUUCAAUUUCUAGU |
| siRNA 3329 | 3329 | CUAGAAAUUGAACCUCGAA | 8819 | UUCGAGGUUCAAUUUCUAG |
| siRNA 3330 | 3330 | UAGAAAUUGAACCUCGAAA | 8820 | UUUCGAGGUUCAAUUUCUA |
| siRNA 3331 | 3331 | AGAAAUUGAACCUCGAAAC | 8821 | GUUUCGAGGUUCAAUUUCU |
| siRNA 3332 | 3332 | GAAAUUGAACCUCGAAACU | 8822 | AGUUUCGAGGUUCAAUUUC |
| siRNA 3333 | 3333 | AAAUUGAACCUCGAAACUG | 8823 | CAGUUUCGAGGUUCAAUUU |
| siRNA 3334 | 3334 | AAUUGAACCUCGAAACUGC | 8824 | GCAGUUUCGAGGUUCAAUU |
| siRNA 3335 | 3335 | AUUGAACCUCGAAACUGCA | 8825 | UGCAGUUUCGAGGUUCAAU |
| siRNA 3336 | 3336 | UUGAACCUCGAAACUGCAA | 8826 | UUGCAGUUUCGAGGUUCAA |
| siRNA 3337 | 3337 | UGAACCUCGAAACUGCAAA | 8827 | UUUGCAGUUUCGAGGUUCA |
| siRNA 3338 | 3338 | GAACCUCGAAACUGCAAAC | 8828 | GUUUGCAGUUUCGAGGUUC |
| siRNA 3339 | 3339 | AACCUCGAAACUGCAAACA | 8829 | UGUUUGCAGUUUCGAGGUU |
| siRNA 3340 | 3340 | ACCUCGAAACUGCAAACAG | 8830 | CUGUUUGCAGUUUCGAGGU |
| siRNA 3341 | 3341 | CCUCGAAACUGCAAACAGC | 8831 | GCUGUUUGCAGUUUCGAGG |
| siRNA 3342 | 3342 | CUCGAAACUGCAAACAGCC | 8832 | GGCUGUUUGCAGUUUCGAG |
| siRNA 3343 | 3343 | UCGAAACUGCAAACAGCCA | 8833 | UGGCUGUUUGCAGUUUCGA |
| siRNA 3344 | 3344 | CGAAACUGCAAACAGCCAC | 8834 | GUGGCUGUUUGCAGUUUCG |
| siRNA 3345 | 3345 | GAAACUGCAAACAGCCACC | 8835 | GGUGGCUGUUUGCAGUUUC |
| siRNA 3346 | 3346 | AAACUGCAAACAGCCACCU | 8836 | AGGUGGCUGUUUGCAGUUU |
| siRNA 3347 | 3347 | AACUGCAAACAGCCACCUC | 8837 | GAGGUGGCUGUUUGCAGUU |
| siRNA 3348 | 3348 | ACUGCAAACAGCCACCUCG | 8838 | CGAGGUGGCUGUUUGCAGU |
| siRNA 3349 | 3349 | CUGCAAACAGCCACCUCGA | 8839 | UCGAGGUGGCUGUUUGCAG |
| siRNA 3350 | 3350 | UGCAAACAGCCACCUCGAU | 8840 | AUCGAGGUGGCUGUUUGCA |
| siRNA 3351 | 3351 | GCAAACAGCCACCUCGAUU | 8841 | AAUCGAGGUGGCUGUUUGC |
| siRNA 3352 | 3352 | CAAACAGCCACCUCGAUUU | 8842 | AAAUCGAGGUGGCUGUUUG |
| siRNA 3353 | 3353 | AAACAGCCACCUCGAUUUC | 8843 | GAAAUCGAGGUGGCUGUUU |
| siRNA 3354 | 3354 | AACAGCCACCUCGAUUUCC | 8844 | GGAAAUCGAGGUGGCUGUU |
| siRNA 3355 | 3355 | ACAGCCACCUCGAUUUCCU | 8845 | AGGAAAUCGAGGUGGCUGU |
| siRNA 3356 | 3356 | CAGCCACCUCGAUUUCCUA | 8846 | UAGGAAAUCGAGGUGGCUG |
| siRNA 3357 | 3357 | AGCCACCUCGAUUUCCUAG | 8847 | CUAGGAAAUCGAGGUGGCU |
| siRNA 3358 | 3358 | GCCACCUCGAUUUCCUAGG | 8848 | CCUAGGAAAUCGAGGUGGC |
| siRNA 3359 | 3359 | CCACCUCGAUUUCCUAGGA | 8849 | UCCUAGGAAAUCGAGGUGG |
| siRNA 3360 | 3360 | CACCUCGAUUUCCUAGGAA | 8850 | UUCCUAGGAAAUCGAGGUG |
| siRNA 3361 | 3361 | ACCUCGAUUUCCUAGGAAA | 8851 | UUUCCUAGGAAAUCGAGGU |
| siRNA 3362 | 3362 | CCUCGAUUUCCUAGGAAAU | 8852 | AUUUCCUAGGAAAUCGAGG |
| siRNA 3363 | 3363 | CUCGAUUUCCUAGGAAAUC | 8853 | GAUUUCCUAGGAAAUCGAG |
| siRNA 3364 | 3364 | UCGAUUUCCUAGGAAAUCA | 8854 | UGAUUUCCUAGGAAAUCGA |
| siRNA 3365 | 3365 | CGAUUUCCUAGGAAAUCAA | 8855 | UUGAUUUCCUAGGAAAUCG |
| siRNA 3366 | 3366 | GAUUUCCUAGGAAAUCAAC | 8856 | GUUGAUUUCCUAGGAAAUC |
| siRNA 3367 | 3367 | AUUUCCUAGGAAAUCAACU | 8857 | AGUUGAUUUCCUAGGAAAU |
| siRNA 3368 | 3368 | UUUCCUAGGAAAUCAACUU | 8858 | AAGUUGAUUUCCUAGGAAA |
| siRNA 3369 | 3369 | UUCCUAGGAAAUCAACUUU | 8859 | AAAGUUGAUUUCCUAGGAA |
| siRNA 3370 | 3370 | UCCUAGGAAAUCAACUUUU | 8860 | AAAAGUUGAUUUCCUAGGA |
| siRNA 3371 | 3371 | CCUAGGAAAUCAACUUUUU | 8861 | AAAAAGUUGAUUUCCUAGG |
| siRNA 3372 | 3372 | CUAGGAAAUCAACUUUUUC | 8862 | GAAAAAGUUGAUUUCCUAG |
| siRNA 3373 | 3373 | UAGGAAAUCAACUUUUUCC | 8863 | GGAAAAAGUUGAUUUCCUA |
| siRNA 3374 | 3374 | AGGAAAUCAACUUUUUCCU | 8864 | AGGAAAAAGUUGAUUUCCU |
| siRNA 3375 | 3375 | GGAAAUCAACUUUUUCCUU | 8865 | AAGGAAAAAGUUGAUUUCC |
| siRNA 3376 | 3376 | GAAAUCAACUUUUUCCUUA | 8866 | UAAGGAAAAAGUUGAUUUC |
| siRNA 3377 | 3377 | AAAUCAACUUUUUCCUUAA | 8867 | UUAAGGAAAAAGUUGAUUU |
| siRNA 3378 | 3378 | AAUCAACUUUUUCCUUAAA | 8868 | UUUAAGGAAAAAGUUGAUU |
| siRNA 3379 | 3379 | AUCAACUUUUUCCUUAAAA | 8869 | UUUUAAGGAAAAAGUUGAU |
| siRNA 3380 | 3380 | UCAACUUUUUCCUUAAAAU | 8870 | AUUUUAAGGAAAAAGUUGA |
| siRNA 3381 | 3381 | CAACUUUUUCCUUAAAAUC | 8871 | GAUUUUAAGGAAAAAGUUG |
| siRNA 3382 | 3382 | AACUUUUUCCUUAAAAUCU | 8872 | AGAUUUUAAGGAAAAAGUU |
| siRNA 3383 | 3383 | ACUUUUUCCUUAAAAUCUC | 8873 | GAGAUUUUAAGGAAAAAGU |
| siRNA 3384 | 3384 | CUUUUUCCUUAAAAUCUCC | 8874 | GGAGAUUUUAAGGAAAAAG |
| siRNA 3385 | 3385 | UUUUUCCUUAAAAUCUCCU | 8875 | AGGAGAUUUUAAGGAAAAA |
| siRNA 3386 | 3386 | UUUUCCUUAAAAUCUCCUG | 8876 | CAGGAGAUUUUAAGGAAAA |
| siRNA 3387 | 3387 | UUUCCUUAAAAUCUCCUGG | 8877 | CCAGGAGAUUUUAAGGAAA |
| siRNA 3388 | 3388 | UUCCUUAAAAUCUCCUGGA | 8878 | UCCAGGAGAUUUUAAGGAA |
| siRNA 3389 | 3389 | UCCUUAAAAUCUCCUGGAA | 8879 | UUCCAGGAGAUUUUAAGGA |
| siRNA 3390 | 3390 | CCUUAAAAUCUCCUGGAAU | 8880 | AUUCCAGGAGAUUUUAAGG |
| siRNA 3391 | 3391 | CUUAAAAUCUCCUGGAAUA | 8881 | UAUUCCAGGAGAUUUUAAG |
| siRNA 3392 | 3392 | UUAAAAUCUCCUGGAAUAA | 8882 | UUAUUCCAGGAGAUUUUAA |
| siRNA 3393 | 3393 | UAAAAUCUCCUGGAAUAAG | 8883 | CUUAUUCCAGGAGAUUUUA |
| siRNA 3394 | 3394 | AAAAUCUCCUGGAAUAAGG | 8884 | CCUUAUUCCAGGAGAUUUU |
| siRNA 3395 | 3395 | AAAUCUCCUGGAAUAAGGC | 8885 | GCCUUAUUCCAGGAGAUUU |
| siRNA 3396 | 3396 | AAUCUCCUGGAAUAAGGCC | 8886 | GGCCUUAUUCCAGGAGAUU |
| siRNA 3397 | 3397 | AUCUCCUGGAAUAAGGCCU | 8887 | AGGCCUUAUUCCAGGAGAU |
| siRNA 3398 | 3398 | UCUCCUGGAAUAAGGCCUA | 8888 | UAGGCCUUAUUCCAGGAGA |
| siRNA 3399 | 3399 | CUCCUGGAAUAAGGCCUAA | 8889 | UUAGGCCUUAUUCCAGGAG |
| siRNA 3400 | 3400 | UCCUGGAAUAAGGCCUAAC | 8890 | GUUAGGCCUUAUUCCAGGA |
| siRNA 3401 | 3401 | CCUGGAAUAAGGCCUAACA | 8891 | UGUUAGGCCUUAUUCCAGG |
| siRNA 3402 | 3402 | CUGGAAUAAGGCCUAACAC | 8892 | GUGUUAGGCCUUAUUCCAG |
| siRNA 3403 | 3403 | UGGAAUAAGGCCUAACACA | 8893 | UGUGUUAGGCCUUAUUCCA |
| siRNA 3404 | 3404 | GGAAUAAGGCCUAACACAG | 8894 | CUGUGUUAGGCCUUAUUCC |
| siRNA 3405 | 3405 | GAAUAAGGCCUAACACAGG | 8895 | CCUGUGUUAGGCCUUAUUC |
| siRNA 3406 | 3406 | AAUAAGGCCUAACACAGGC | 8896 | GCCUGUGUUAGGCCUUAUU |
| siRNA 3407 | 3407 | AUAAGGCCUAACACAGGCC | 8897 | GGCCUGUGUUAGGCCUUAU |
| siRNA 3408 | 3408 | UAAGGCCUAACACAGGCCG | 8898 | CGGCCUGUGUUAGGCCUUA |
| siRNA 3409 | 3409 | AAGGCCUAACACAGGCCGA | 8899 | UCGGCCUGUGUUAGGCCUU |
| siRNA 3410 | 3410 | AGGCCUAACACAGGCCGAC | 8900 | GUCGGCCUGUGUUAGGCCU |
| siRNA 3411 | 3411 | GGCCUAACACAGGCCGACA | 8901 | UGUCGGCCUGUGUUAGGCC |
| siRNA 3412 | 3412 | GCCUAACACAGGCCGACAU | 8902 | AUGUCGGCCUGUGUUAGGC |
| siRNA 3413 | 3413 | CCUAACACAGGCCGACAUG | 8903 | CAUGUCGGCCUGUGUUAGG |
| siRNA 3414 | 3414 | CUAACACAGGCCGACAUGG | 8904 | CCAUGUCGGCCUGUGUUAG |
| siRNA 3415 | 3415 | UAACACAGGCCGACAUGGC | 8905 | GCCAUGUCGGCCUGUGUUA |
| siRNA 3416 | 3416 | AACACAGGCCGACAUGGCU | 8906 | AGCCAUGUCGGCCUGUGUU |
| siRNA 3417 | 3417 | ACACAGGCCGACAUGGCUC | 8907 | GAGCCAUGUCGGCCUGUGU |
| siRNA 3418 | 3418 | CACAGGCCGACAUGGCUCU | 8908 | AGAGCCAUGUCGGCCUGUG |
| siRNA 3419 | 3419 | ACAGGCCGACAUGGCUCUA | 8909 | UAGAGCCAUGUCGGCCUGU |
| siRNA 3420 | 3420 | CAGGCCGACAUGGCUCUAC | 8910 | GUAGAGCCAUGUCGGCCUG |
| siRNA 3421 | 3421 | AGGCCGACAUGGCUCUACC | 8911 | GGUAGAGCCAUGUCGGCCU |
| siRNA 3422 | 3422 | GGCCGACAUGGCUCUACCU | 8912 | AGGUAGAGCCAUGUCGGCC |
| siRNA 3423 | 3423 | GCCGACAUGGCUCUACCUC | 8913 | GAGGUAGAGCCAUGUCGGC |
| siRNA 3424 | 3424 | CCGACAUGGCUCUACCUCA | 8914 | UGAGGUAGAGCCAUGUCGG |
| siRNA 3425 | 3425 | CGACAUGGCUCUACCUCAG | 8915 | CUGAGGUAGAGCCAUGUCG |
| siRNA 3426 | 3426 | GACAUGGCUCUACCUCAGG | 8916 | CCUGAGGUAGAGCCAUGUC |
| siRNA 3427 | 3427 | ACAUGGCUCUACCUCAGGU | 8917 | ACCUGAGGUAGAGCCAUGU |
| siRNA 3428 | 3428 | CAUGGCUCUACCUCAGGUA | 8918 | UACCUGAGGUAGAGCCAUG |
| siRNA 3429 | 3429 | AUGGCUCUACCUCAGGUAC | 8919 | GUACCUGAGGUAGAGCCAU |
| siRNA 3430 | 3430 | UGGCUCUACCUCAGGUACU | 8920 | AGUACCUGAGGUAGAGCCA |
| siRNA 3431 | 3431 | GGCUCUACCUCAGGUACUU | 8921 | AAGUACCUGAGGUAGAGCC |
| siRNA 3432 | 3432 | GCUCUACCUCAGGUACUUU | 8922 | AAAGUACCUGAGGUAGAGC |
| siRNA 3433 | 3433 | CUCUACCUCAGGUACUUUA | 8923 | UAAAGUACCUGAGGUAGAG |
| siRNA 3434 | 3434 | UCUACCUCAGGUACUUUAC | 8924 | GUAAAGUACCUGAGGUAGA |
| siRNA 3435 | 3435 | CUACCUCAGGUACUUUACG | 8925 | CGUAAAGUACCUGAGGUAG |
| siRNA 3436 | 3436 | UACCUCAGGUACUUUACGA | 8926 | UCGUAAAGUACCUGAGGUA |
| siRNA 3437 | 3437 | ACCUCAGGUACUUUACGAG | 8927 | CUCGUAAAGUACCUGAGGU |
| siRNA 3438 | 3438 | CCUCAGGUACUUUACGAGG | 8928 | CCUCGUAAAGUACCUGAGG |
| siRNA 3439 | 3439 | CUCAGGUACUUUACGAGGU | 8929 | ACCUCGUAAAGUACCUGAG |
| siRNA 3440 | 3440 | UCAGGUACUUUACGAGGUC | 8930 | GACCUCGUAAAGUACCUGA |
| siRNA 3441 | 3441 | CAGGUACUUUACGAGGUCA | 8931 | UGACCUCGUAAAGUACCUG |
| siRNA 3442 | 3442 | AGGUACUUUACGAGGUCAC | 8932 | GUGACCUCGUAAAGUACCU |
| siRNA 3443 | 3443 | GGUACUUUACGAGGUCACC | 8933 | GGUGACCUCGUAAAGUACC |
| siRNA 3444 | 3444 | GUACUUUACGAGGUCACCC | 8934 | GGGUGACCUCGUAAAGUAC |
| siRNA 3445 | 3445 | UACUUUACGAGGUCACCCA | 8935 | UGGGUGACCUCGUAAAGUA |
| siRNA 3446 | 3446 | ACUUUACGAGGUCACCCAA | 8936 | UUGGGUGACCUCGUAAAGU |
| siRNA 3447 | 3447 | CUUUACGAGGUCACCCAAC | 8937 | GUUGGGUGACCUCGUAAAG |
| siRNA 3448 | 3448 | UUUACGAGGUCACCCAACA | 8938 | UGUUGGGUGACCUCGUAAA |
| siRNA 3449 | 3449 | UUACGAGGUCACCCAACAC | 8939 | GUGUUGGGUGACCUCGUAA |
| siRNA 3450 | 3450 | UACGAGGUCACCCAACACC | 8940 | GGUGUUGGGUGACCUCGUA |
| siRNA 3451 | 3451 | ACGAGGUCACCCAACACCA | 8941 | UGGUGUUGGGUGACCUCGU |
| siRNA 3452 | 3452 | CGAGGUCACCCAACACCAU | 8942 | AUGGUGUUGGGUGACCUCG |
| siRNA 3453 | 3453 | GAGGUCACCCAACACCAUU | 8943 | AAUGGUGUUGGGUGACCUC |
| siRNA 3454 | 3454 | AGGUCACCCAACACCAUUA | 8944 | UAAUGGUGUUGGGUGACCU |
| siRNA 3455 | 3455 | GGUCACCCAACACCAUUAG | 8945 | CUAAUGGUGUUGGGUGACC |
| siRNA 3456 | 3456 | GUCACCCAACACCAUUAGA | 8946 | UCUAAUGGUGUUGGGUGAC |
| siRNA 3457 | 3457 | UCACCCAACACCAUUAGAA | 8947 | UUCUAAUGGUGUUGGGUGA |
| siRNA 3458 | 3458 | CACCCAACACCAUUAGAAA | 8948 | UUUCUAAUGGUGUUGGGUG |
| siRNA 3459 | 3459 | ACCCAACACCAUUAGAAAG | 8949 | CUUUCUAAUGGUGUUGGGU |
| siRNA 3460 | 3460 | CCCAACACCAUUAGAAAGA | 8950 | UCUUUCUAAUGGUGUUGGG |
| siRNA 3461 | 3461 | CCAACACCAUUAGAAAGAG | 8951 | CUCUUUCUAAUGGUGUUGG |
| siRNA 3462 | 3462 | CAACACCAUUAGAAAGAGA | 8952 | UCUCUUUCUAAUGGUGUUG |
| siRNA 3463 | 3463 | AACACCAUUAGAAAGAGAA | 8953 | UUCUCUUUCUAAUGGUGUU |
| siRNA 3464 | 3464 | ACACCAUUAGAAAGAGAAC | 8954 | GUUCUCUUUCUAAUGGUGU |
| siRNA 3465 | 3465 | CACCAUUAGAAAGAGAACC | 8955 | GGUUCUCUUUCUAAUGGUG |
| siRNA 3466 | 3466 | ACCAUUAGAAAGAGAACCA | 8956 | UGGUUCUCUUUCUAAUGGU |
| siRNA 3467 | 3467 | CCAUUAGAAAGAGAACCAU | 8957 | AUGGUUCUCUUUCUAAUGG |
| siRNA 3468 | 3468 | CAUUAGAAAGAGAACCAUG | 8958 | CAUGGUUCUCUUUCUAAUG |
| siRNA 3469 | 3469 | AUUAGAAAGAGAACCAUGU | 8959 | ACAUGGUUCUCUUUCUAAU |
| siRNA 3470 | 3470 | UUAGAAAGAGAACCAUGUA | 8960 | UACAUGGUUCUCUUUCUAA |
| siRNA 3471 | 3471 | UAGAAAGAGAACCAUGUAA | 8961 | UUACAUGGUUCUCUUUCUA |
| siRNA 3472 | 3472 | AGAAAGAGAACCAUGUAAA | 8962 | UUUACAUGGUUCUCUUUCU |
| siRNA 3473 | 3473 | GAAAGAGAACCAUGUAAAA | 8963 | UUUUACAUGGUUCUCUUUC |
| siRNA 3474 | 3474 | AAAGAGAACCAUGUAAAAU | 8964 | AUUUUACAUGGUUCUCUUU |
| siRNA 3475 | 3475 | AAGAGAACCAUGUAAAAUA | 8965 | UAUUUUACAUGGUUCUCUU |
| siRNA 3476 | 3476 | AGAGAACCAUGUAAAAUAA | 8966 | UUAUUUUACAUGGUUCUCU |
| siRNA 3477 | 3477 | GAGAACCAUGUAAAAUAAG | 8967 | CUUAUUUUACAUGGUUCUC |
| siRNA 3478 | 3478 | AGAACCAUGUAAAAUAAGC | 8968 | GCUUAUUUUACAUGGUUCU |
| siRNA 3479 | 3479 | GAACCAUGUAAAAUAAGCU | 8969 | AGCUUAUUUUACAUGGUUC |
| siRNA 3480 | 3480 | AACCAUGUAAAAUAAGCUU | 8970 | AAGCUUAUUUUACAUGGUU |
| siRNA 3481 | 3481 | ACCAUGUAAAAUAAGCUUU | 8971 | AAAGCUUAUUUUACAUGGU |
| siRNA 3482 | 3482 | CCAUGUAAAAUAAGCUUUA | 8972 | UAAAGCUUAUUUUACAUGG |
| siRNA 3483 | 3483 | CAUGUAAAAUAAGCUUUAG | 8973 | CUAAAGCUUAUUUUACAUG |
| siRNA 3484 | 3484 | AUGUAAAAUAAGCUUUAGU | 8974 | ACUAAAGCUUAUUUUACAU |
| siRNA 3485 | 3485 | UGUAAAAUAAGCUUUAGUC | 8975 | GACUAAAGCUUAUUUUACA |
| siRNA 3486 | 3486 | GUAAAAUAAGCUUUAGUCG | 8976 | CGACUAAAGCUUAUUUUAC |
| siRNA 3487 | 3487 | UAAAAUAAGCUUUAGUCGG | 8977 | CCGACUAAAGCUUAUUUUA |
| siRNA 3488 | 3488 | AAAAUAAGCUUUAGUCGGA | 8978 | UCCGACUAAAGCUUAUUUU |
| siRNA 3489 | 3489 | AAAUAAGCUUUAGUCGGAU | 8979 | AUCCGACUAAAGCUUAUUU |
| siRNA 3490 | 3490 | AAUAAGCUUUAGUCGGAUU | 8980 | AAUCCGACUAAAGCUUAUU |
| siRNA 3491 | 3491 | AUAAGCUUUAGUCGGAUUG | 8981 | CAAUCCGACUAAAGCUUAU |
| siRNA 3492 | 3492 | UAAGCUUUAGUCGGAUUGC | 8982 | GCAAUCCGACUAAAGCUUA |
| siRNA 3493 | 3493 | AAGCUUUAGUCGGAUUGCU | 8983 | AGCAAUCCGACUAAAGCUU |
| siRNA 3494 | 3494 | AGCUUUAGUCGGAUUGCUG | 8984 | CAGCAAUCCGACUAAAGCU |
| siRNA 3495 | 3495 | GCUUUAGUCGGAUUGCUGA | 8985 | UCAGCAAUCCGACUAAAGC |
| siRNA 3496 | 3496 | CUUUAGUCGGAUUGCUGAA | 8986 | UUCAGCAAUCCGACUAAAG |
| siRNA 3497 | 3497 | UUUAGUCGGAUUGCUGAAA | 8987 | UUUCAGCAAUCCGACUAAA |
| siRNA 3498 | 3498 | UUAGUCGGAUUGCUGAAAC | 8988 | GUUUCAGCAAUCCGACUAA |
| siRNA 3499 | 3499 | UAGUCGGAUUGCUGAAACU | 8989 | AGUUUCAGCAAUCCGACUA |
| siRNA 3500 | 3500 | AGUCGGAUUGCUGAAACUG | 8990 | CAGUUUCAGCAAUCCGACU |
| siRNA 3501 | 3501 | GUCGGAUUGCUGAAACUGA | 8991 | UCAGUUUCAGCAAUCCGAC |
| siRNA 3502 | 3502 | UCGGAUUGCUGAAACUGAG | 8992 | CUCAGUUUCAGCAAUCCGA |
| siRNA 3503 | 3503 | CGGAUUGCUGAAACUGAGC | 8993 | GCUCAGUUUCAGCAAUCCG |
| siRNA 3504 | 3504 | GGAUUGCUGAAACUGAGCU | 8994 | AGCUCAGUUUCAGCAAUCC |
| siRNA 3505 | 3505 | GAUUGCUGAAACUGAGCUG | 8995 | CAGCUCAGUUUCAGCAAUC |
| siRNA 3506 | 3506 | AUUGCUGAAACUGAGCUGG | 8996 | CCAGCUCAGUUUCAGCAAU |
| siRNA 3507 | 3507 | UUGCUGAAACUGAGCUGGA | 8997 | UCCAGCUCAGUUUCAGCAA |
| siRNA 3508 | 3508 | UGCUGAAACUGAGCUGGAA | 8998 | UUCCAGCUCAGUUUCAGCA |
| siRNA 3509 | 3509 | GCUGAAACUGAGCUGGAAU | 8999 | AUUCCAGCUCAGUUUCAGC |
| siRNA 3510 | 3510 | CUGAAACUGAGCUGGAAUC | 9000 | GAUUCCAGCUCAGUUUCAG |
| siRNA 3511 | 3511 | UGAAACUGAGCUGGAAUCA | 9001 | UGAUUCCAGCUCAGUUUCA |
| siRNA 3512 | 3512 | GAAACUGAGCUGGAAUCAA | 9002 | UUGAUUCCAGCUCAGUUUC |
| siRNA 3513 | 3513 | AAACUGAGCUGGAAUCAAC | 9003 | GUUGAUUCCAGCUCAGUUU |
| siRNA 3514 | 3514 | AACUGAGCUGGAAUCAACA | 9004 | UGUUGAUUCCAGCUCAGUU |
| siRNA 3515 | 3515 | ACUGAGCUGGAAUCAACAG | 9005 | CUGUUGAUUCCAGCUCAGU |
| siRNA 3516 | 3516 | CUGAGCUGGAAUCAACAGU | 9006 | ACUGUUGAUUCCAGCUCAG |
| siRNA 3517 | 3517 | UGAGCUGGAAUCAACAGUG | 9007 | CACUGUUGAUUCCAGCUCA |
| siRNA 3518 | 3518 | GAGCUGGAAUCAACAGUGU | 9008 | ACACUGUUGAUUCCAGCUC |
| siRNA 3519 | 3519 | AGCUGGAAUCAACAGUGUC | 9009 | GACACUGUUGAUUCCAGCU |
| siRNA 3520 | 3520 | GCUGGAAUCAACAGUGUCA | 9010 | UGACACUGUUGAUUCCAGC |
| siRNA 3521 | 3521 | CUGGAAUCAACAGUGUCAG | 9011 | CUGACACUGUUGAUUCCAG |
| siRNA 3522 | 3522 | UGGAAUCAACAGUGUCAGC | 9012 | GCUGACACUGUUGAUUCCA |
| siRNA 3523 | 3523 | GGAAUCAACAGUGUCAGCA | 9013 | UGCUGACACUGUUGAUUCC |
| siRNA 3524 | 3524 | GAAUCAACAGUGUCAGCAC | 9014 | GUGCUGACACUGUUGAUUC |
| siRNA 3525 | 3525 | AAUCAACAGUGUCAGCACC | 9015 | GGUGCUGACACUGUUGAUU |
| siRNA 3526 | 3526 | AUCAACAGUGUCAGCACCA | 9016 | UGGUGCUGACACUGUUGAU |
| siRNA 3527 | 3527 | UCAACAGUGUCAGCACCAA | 9017 | UUGGUGCUGACACUGUUGA |
| siRNA 3528 | 3528 | CAACAGUGUCAGCACCAAC | 9018 | GUUGGUGCUGACACUGUUG |
| siRNA 3529 | 3529 | AACAGUGUCAGCACCAACC | 9019 | GGUUGGUGCUGACACUGUU |
| siRNA 3530 | 3530 | ACAGUGUCAGCACCAACCU | 9020 | AGGUUGGUGCUGACACUGU |
| siRNA 3531 | 3531 | CAGUGUCAGCACCAACCUC | 9021 | GAGGUUGGUGCUGACACUG |
| siRNA 3532 | 3532 | AGUGUCAGCACCAACCUCU | 9022 | AGAGGUUGGUGCUGACACU |
| siRNA 3533 | 3533 | GUGUCAGCACCAACCUCUC | 9023 | GAGAGGUUGGUGCUGACAC |
| siRNA 3534 | 3534 | UGUCAGCACCAACCUCUCC | 9024 | GGAGAGGUUGGUGCUGACA |
| siRNA 3535 | 3535 | GUCAGCACCAACCUCUCCA | 9025 | UGGAGAGGUUGGUGCUGAC |
| siRNA 3536 | 3536 | UCAGCACCAACCUCUCCAA | 9026 | UUGGAGAGGUUGGUGCUGA |
| siRNA 3537 | 3537 | CAGCACCAACCUCUCCAAA | 9027 | UUUGGAGAGGUUGGUGCUG |
| siRNA 3538 | 3538 | AGCACCAACCUCUCCAAAU | 9028 | AUUUGGAGAGGUUGGUGCU |
| siRNA 3539 | 3539 | GCACCAACCUCUCCAAAUA | 9029 | UAUUUGGAGAGGUUGGUGC |
| siRNA 3540 | 3540 | CACCAACCUCUCCAAAUAC | 9030 | GUAUUUGGAGAGGUUGGUG |
| siRNA 3541 | 3541 | ACCAACCUCUCCAAAUACA | 9031 | UGUAUUUGGAGAGGUUGGU |
| siRNA 3542 | 3542 | CCAACCUCUCCAAAUACAC | 9032 | GUGUAUUUGGAGAGGUUGG |
| siRNA 3543 | 3543 | CAACCUCUCCAAAUACACC | 9033 | GGUGUAUUUGGAGAGGUUG |
| siRNA 3544 | 3544 | AACCUCUCCAAAUACACCA | 9034 | UGGUGUAUUUGGAGAGGUU |
| siRNA 3545 | 3545 | ACCUCUCCAAAUACACCAU | 9035 | AUGGUGUAUUUGGAGAGGU |
| siRNA 3546 | 3546 | CCUCUCCAAAUACACCAUC | 9036 | GAUGGUGUAUUUGGAGAGG |
| siRNA 3547 | 3547 | CUCUCCAAAUACACCAUCU | 9037 | AGAUGGUGUAUUUGGAGAG |
| siRNA 3548 | 3548 | UCUCCAAAUACACCAUCUA | 9038 | UAGAUGGUGUAUUUGGAGA |
| siRNA 3549 | 3549 | CUCCAAAUACACCAUCUAC | 9039 | GUAGAUGGUGUAUUUGGAG |
| siRNA 3550 | 3550 | UCCAAAUACACCAUCUACU | 9040 | AGUAGAUGGUGUAUUUGGA |
| siRNA 3551 | 3551 | CCAAAUACACCAUCUACUC | 9041 | GAGUAGAUGGUGUAUUUGG |
| siRNA 3552 | 3552 | CAAAUACACCAUCUACUCC | 9042 | GGAGUAGAUGGUGUAUUUG |
| siRNA 3553 | 3553 | AAAUACACCAUCUACUCCA | 9043 | UGGAGUAGAUGGUGUAUUU |
| siRNA 3554 | 3554 | AAUACACCAUCUACUCCAC | 9044 | GUGGAGUAGAUGGUGUAUU |
| siRNA 3555 | 3555 | AUACACCAUCUACUCCACC | 9045 | GGUGGAGUAGAUGGUGUAU |
| siRNA 3556 | 3556 | UACACCAUCUACUCCACCA | 9046 | UGGUGGAGUAGAUGGUGUA |
| siRNA 3557 | 3557 | ACACCAUCUACUCCACCAG | 9047 | CUGGUGGAGUAGAUGGUGU |
| siRNA 3558 | 3558 | CACCAUCUACUCCACCAGU | 9048 | ACUGGUGGAGUAGAUGGUG |
| siRNA 3559 | 3559 | ACCAUCUACUCCACCAGUA | 9049 | UACUGGUGGAGUAGAUGGU |
| siRNA 3560 | 3560 | CCAUCUACUCCACCAGUAU | 9050 | AUACUGGUGGAGUAGAUGG |
| siRNA 3561 | 3561 | CAUCUACUCCACCAGUAUC | 9051 | GAUACUGGUGGAGUAGAUG |
| siRNA 3562 | 3562 | AUCUACUCCACCAGUAUCU | 9052 | AGAUACUGGUGGAGUAGAU |
| siRNA 3563 | 3563 | UCUACUCCACCAGUAUCUG | 9053 | CAGAUACUGGUGGAGUAGA |
| siRNA 3564 | 3564 | CUACUCCACCAGUAUCUGC | 9054 | GCAGAUACUGGUGGAGUAG |
| siRNA 3565 | 3565 | UACUCCACCAGUAUCUGCU | 9055 | AGCAGAUACUGGUGGAGUA |
| siRNA 3566 | 3566 | ACUCCACCAGUAUCUGCUU | 9056 | AAGCAGAUACUGGUGGAGU |
| siRNA 3567 | 3567 | CUCCACCAGUAUCUGCUUC | 9057 | GAAGCAGAUACUGGUGGAG |
| siRNA 3568 | 3568 | UCCACCAGUAUCUGCUUCU | 9058 | AGAAGCAGAUACUGGUGGA |
| siRNA 3569 | 3569 | CCACCAGUAUCUGCUUCUU | 9059 | AAGAAGCAGAUACUGGUGG |
| siRNA 3570 | 3570 | CACCAGUAUCUGCUUCUUC | 9060 | GAAGAAGCAGAUACUGGUG |
| siRNA 3571 | 3571 | ACCAGUAUCUGCUUCUUCA | 9061 | UGAAGAAGCAGAUACUGGU |
| siRNA 3572 | 3572 | CCAGUAUCUGCUUCUUCAG | 9062 | CUGAAGAAGCAGAUACUGG |
| siRNA 3573 | 3573 | CAGUAUCUGCUUCUUCAGA | 9063 | UCUGAAGAAGCAGAUACUG |
| siRNA 3574 | 3574 | AGUAUCUGCUUCUUCAGAC | 9064 | GUCUGAAGAAGCAGAUACU |
| siRNA 3575 | 3575 | GUAUCUGCUUCUUCAGACC | 9065 | GGUCUGAAGAAGCAGAUAC |
| siRNA 3576 | 3576 | UAUCUGCUUCUUCAGACCU | 9066 | AGGUCUGAAGAAGCAGAUA |
| siRNA 3577 | 3577 | AUCUGCUUCUUCAGACCUU | 9067 | AAGGUCUGAAGAAGCAGAU |
| siRNA 3578 | 3578 | UCUGCUUCUUCAGACCUUA | 9068 | UAAGGUCUGAAGAAGCAGA |
| siRNA 3579 | 3579 | CUGCUUCUUCAGACCUUAG | 9069 | CUAAGGUCUGAAGAAGCAG |
| siRNA 3580 | 3580 | UGCUUCUUCAGACCUUAGU | 9070 | ACUAAGGUCUGAAGAAGCA |
| siRNA 3581 | 3581 | GCUUCUUCAGACCUUAGUG | 9071 | CACUAAGGUCUGAAGAAGC |
| siRNA 3582 | 3582 | CUUCUUCAGACCUUAGUGU | 9072 | ACACUAAGGUCUGAAGAAG |
| siRNA 3583 | 3583 | UUCUUCAGACCUUAGUGUA | 9073 | UACACUAAGGUCUGAAGAA |
| siRNA 3584 | 3584 | UCUUCAGACCUUAGUGUAU | 9074 | AUACACUAAGGUCUGAAGA |
| siRNA 3585 | 3585 | CUUCAGACCUUAGUGUAUU | 9075 | AAUACACUAAGGUCUGAAG |
| siRNA 3586 | 3586 | UUCAGACCUUAGUGUAUUU | 9076 | AAAUACACUAAGGUCUGAA |
| siRNA 3587 | 3587 | UCAGACCUUAGUGUAUUUU | 9077 | AAAAUACACUAAGGUCUGA |
| siRNA 3588 | 3588 | CAGACCUUAGUGUAUUUUU | 9078 | AAAAAUACACUAAGGUCUG |
| siRNA 3589 | 3589 | AGACCUUAGUGUAUUUUUA | 9079 | UAAAAAUACACUAAGGUCU |
| siRNA 3590 | 3590 | GACCUUAGUGUAUUUUUAG | 9080 | CUAAAAAUACACUAAGGUC |
| siRNA 3591 | 3591 | ACCUUAGUGUAUUUUUAGA | 9081 | UCUAAAAAUACACUAAGGU |
| siRNA 3592 | 3592 | CCUUAGUGUAUUUUUAGAU | 9082 | AUCUAAAAAUACACUAAGG |
| siRNA 3593 | 3593 | CUUAGUGUAUUUUUAGAUG | 9083 | CAUCUAAAAAUACACUAAG |
| siRNA 3594 | 3594 | UUAGUGUAUUUUUAGAUGU | 9084 | ACAUCUAAAAAUACACUAA |
| siRNA 3595 | 3595 | UAGUGUAUUUUUAGAUGUG | 9085 | CACAUCUAAAAAUACACUA |
| siRNA 3596 | 3596 | AGUGUAUUUUUAGAUGUGG | 9086 | CCACAUCUAAAAAUACACU |
| siRNA 3597 | 3597 | GUGUAUUUUUAGAUGUGGA | 9087 | UCCACAUCUAAAAAUACAC |
| siRNA 3598 | 3598 | UGUAUUUUUAGAUGUGGAU | 9088 | AUCCACAUCUAAAAAUACA |
| siRNA 3599 | 3599 | GUAUUUUUAGAUGUGGAUC | 9089 | GAUCCACAUCUAAAAAUAC |
| siRNA 3600 | 3600 | UAUUUUUAGAUGUGGAUCU | 9090 | AGAUCCACAUCUAAAAAUA |
| siRNA 3601 | 3601 | AUUUUUAGAUGUGGAUCUC | 9091 | GAGAUCCACAUCUAAAAAU |
| siRNA 3602 | 3602 | UUUUUAGAUGUGGAUCUCA | 9092 | UGAGAUCCACAUCUAAAAA |
| siRNA 3603 | 3603 | UUUUAGAUGUGGAUCUCAA | 9093 | UUGAGAUCCACAUCUAAAA |
| siRNA 3604 | 3604 | UUUAGAUGUGGAUCUCAAC | 9094 | GUUGAGAUCCACAUCUAAA |
| siRNA 3605 | 3605 | UUAGAUGUGGAUCUCAACA | 9095 | UGUUGAGAUCCACAUCUAA |
| siRNA 3606 | 3606 | UAGAUGUGGAUCUCAACAG | 9096 | CUGUUGAGAUCCACAUCUA |
| siRNA 3607 | 3607 | AGAUGUGGAUCUCAACAGC | 9097 | GCUGUUGAGAUCCACAUCU |
| siRNA 3608 | 3608 | GAUGUGGAUCUCAACAGCU | 9098 | AGCUGUUGAGAUCCACAUC |
| siRNA 3609 | 3609 | AUGUGGAUCUCAACAGCUC | 9099 | GAGCUGUUGAGAUCCACAU |
| siRNA 3610 | 3610 | UGUGGAUCUCAACAGCUCC | 9100 | GGAGCUGUUGAGAUCCACA |
| siRNA 3611 | 3611 | GUGGAUCUCAACAGCUCCU | 9101 | AGGAGCUGUUGAGAUCCAC |
| siRNA 3612 | 3612 | UGGAUCUCAACAGCUCCUG | 9102 | CAGGAGCUGUUGAGAUCCA |
| siRNA 3613 | 3613 | GGAUCUCAACAGCUCCUGU | 9103 | ACAGGAGCUGUUGAGAUCC |
| siRNA 3614 | 3614 | GAUCUCAACAGCUCCUGUG | 9104 | CACAGGAGCUGUUGAGAUC |
| siRNA 3615 | 3615 | AUCUCAACAGCUCCUGUGG | 9105 | CCACAGGAGCUGUUGAGAU |
| siRNA 3616 | 3616 | UCUCAACAGCUCCUGUGGC | 9106 | GCCACAGGAGCUGUUGAGA |
| siRNA 3617 | 3617 | CUCAACAGCUCCUGUGGCA | 9107 | UGCCACAGGAGCUGUUGAG |
| siRNA 3618 | 3618 | UCAACAGCUCCUGUGGCAG | 9108 | CUGCCACAGGAGCUGUUGA |
| siRNA 3619 | 3619 | CAACAGCUCCUGUGGCAGC | 9109 | GCUGCCACAGGAGCUGUUG |
| siRNA 3620 | 3620 | AACAGCUCCUGUGGCAGCA | 9110 | UGCUGCCACAGGAGCUGUU |
| siRNA 3621 | 3621 | ACAGCUCCUGUGGCAGCAA | 9111 | UUGCUGCCACAGGAGCUGU |
| siRNA 3622 | 3622 | CAGCUCCUGUGGCAGCAAU | 9112 | AUUGCUGCCACAGGAGCUG |
| siRNA 3623 | 3623 | AGCUCCUGUGGCAGCAAUA | 9113 | UAUUGCUGCCACAGGAGCU |
| siRNA 3624 | 3624 | GCUCCUGUGGCAGCAAUAG | 9114 | CUAUUGCUGCCACAGGAGC |
| siRNA 3625 | 3625 | CUCCUGUGGCAGCAAUAGC | 9115 | GCUAUUGCUGCCACAGGAG |
| siRNA 3626 | 3626 | UCCUGUGGCAGCAAUAGCA | 9116 | UGCUAUUGCUGCCACAGGA |
| siRNA 3627 | 3627 | CCUGUGGCAGCAAUAGCAU | 9117 | AUGCUAUUGCUGCCACAGG |
| siRNA 3628 | 3628 | CUGUGGCAGCAAUAGCAUC | 9118 | GAUGCUAUUGCUGCCACAG |
| siRNA 3629 | 3629 | UGUGGCAGCAAUAGCAUCU | 9119 | AGAUGCUAUUGCUGCCACA |
| siRNA 3630 | 3630 | GUGGCAGCAAUAGCAUCUU | 9120 | AAGAUGCUAUUGCUGCCAC |
| siRNA 3631 | 3631 | UGGCAGCAAUAGCAUCUUU | 9121 | AAAGAUGCUAUUGCUGCCA |
| siRNA 3632 | 3632 | GGCAGCAAUAGCAUCUUUG | 9122 | CAAAGAUGCUAUUGCUGCC |
| siRNA 3633 | 3633 | GCAGCAAUAGCAUCUUUGC | 9123 | GCAAAGAUGCUAUUGCUGC |
| siRNA 3634 | 3634 | CAGCAAUAGCAUCUUUGCU | 9124 | AGCAAAGAUGCUAUUGCUG |
| siRNA 3635 | 3635 | AGCAAUAGCAUCUUUGCUC | 9125 | GAGCAAAGAUGCUAUUGCU |
| siRNA 3636 | 3636 | GCAAUAGCAUCUUUGCUCC | 9126 | GGAGCAAAGAUGCUAUUGC |
| siRNA 3637 | 3637 | CAAUAGCAUCUUUGCUCCA | 9127 | UGGAGCAAAGAUGCUAUUG |
| siRNA 3638 | 3638 | AAUAGCAUCUUUGCUCCAG | 9128 | CUGGAGCAAAGAUGCUAUU |
| siRNA 3639 | 3639 | AUAGCAUCUUUGCUCCAGU | 9129 | ACUGGAGCAAAGAUGCUAU |
| siRNA 3640 | 3640 | UAGCAUCUUUGCUCCAGUG | 9130 | CACUGGAGCAAAGAUGCUA |
| siRNA 3641 | 3641 | AGCAUCUUUGCUCCAGUGC | 9131 | GCACUGGAGCAAAGAUGCU |
| siRNA 3642 | 3642 | GCAUCUUUGCUCCAGUGCU | 9132 | AGCACUGGAGCAAAGAUGC |
| siRNA 3643 | 3643 | CAUCUUUGCUCCAGUGCUU | 9133 | AAGCACUGGAGCAAAGAUG |
| siRNA 3644 | 3644 | AUCUUUGCUCCAGUGCUUU | 9134 | AAAGCACUGGAGCAAAGAU |
| siRNA 3645 | 3645 | UCUUUGCUCCAGUGCUUUU | 9135 | AAAAGCACUGGAGCAAAGA |
| siRNA 3646 | 3646 | CUUUGCUCCAGUGCUUUUG | 9136 | CAAAAGCACUGGAGCAAAG |
| siRNA 3647 | 3647 | UUUGCUCCAGUGCUUUUGC | 9137 | GCAAAAGCACUGGAGCAAA |
| siRNA 3648 | 3648 | UUGCUCCAGUGCUUUUGCC | 9138 | GGCAAAAGCACUGGAGCAA |
| siRNA 3649 | 3649 | UGCUCCAGUGCUUUUGCCA | 9139 | UGGCAAAAGCACUGGAGCA |
| siRNA 3650 | 3650 | GCUCCAGUGCUUUUGCCAC | 9140 | GUGGCAAAAGCACUGGAGC |
| siRNA 3651 | 3651 | CUCCAGUGCUUUUGCCACA | 9141 | UGUGGCAAAAGCACUGGAG |
| siRNA 3652 | 3652 | UCCAGUGCUUUUGCCACAU | 9142 | AUGUGGCAAAAGCACUGGA |
| siRNA 3653 | 3653 | CCAGUGCUUUUGCCACAUU | 9143 | AAUGUGGCAAAAGCACUGG |
| siRNA 3654 | 3654 | CAGUGCUUUUGCCACAUUC | 9144 | GAAUGUGGCAAAAGCACUG |
| siRNA 3655 | 3655 | AGUGCUUUUGCCACAUUCA | 9145 | UGAAUGUGGCAAAAGCACU |
| siRNA 3656 | 3656 | GUGCUUUUGCCACAUUCAA | 9146 | UUGAAUGUGGCAAAAGCAC |
| siRNA 3657 | 3657 | UGCUUUUGCCACAUUCAAA | 9147 | UUUGAAUGUGGCAAAAGCA |
| siRNA 3658 | 3658 | GCUUUUGCCACAUUCAAAG | 9148 | CUUUGAAUGUGGCAAAAGC |
| siRNA 3659 | 3659 | CUUUUGCCACAUUCAAAGU | 9149 | ACUUUGAAUGUGGCAAAAG |
| siRNA 3660 | 3660 | UUUUGCCACAUUCAAAGUC | 9150 | GACUUUGAAUGUGGCAAAA |
| siRNA 3661 | 3661 | UUUGCCACAUUCAAAGUCU | 9151 | AGACUUUGAAUGUGGCAAA |
| siRNA 3662 | 3662 | UUGCCACAUUCAAAGUCUU | 9152 | AAGACUUUGAAUGUGGCAA |
| siRNA 3663 | 3663 | UGCCACAUUCAAAGUCUUU | 9153 | AAAGACUUUGAAUGUGGCA |
| siRNA 3664 | 3664 | GCCACAUUCAAAGUCUUUC | 9154 | GAAAGACUUUGAAUGUGGC |
| siRNA 3665 | 3665 | CCACAUUCAAAGUCUUUCU | 9155 | AGAAAGACUUUGAAUGUGG |
| siRNA 3666 | 3666 | CACAUUCAAAGUCUUUCUU | 9156 | AAGAAAGACUUUGAAUGUG |
| siRNA 3667 | 3667 | ACAUUCAAAGUCUUUCUUU | 9157 | AAAGAAAGACUUUGAAUGU |
| siRNA 3668 | 3668 | CAUUCAAAGUCUUUCUUUA | 9158 | UAAAGAAAGACUUUGAAUG |
| siRNA 3669 | 3669 | AUUCAAAGUCUUUCUUUAG | 9159 | CUAAAGAAAGACUUUGAAU |
| siRNA 3670 | 3670 | UUCAAAGUCUUUCUUUAGU | 9160 | ACUAAAGAAAGACUUUGAA |
| siRNA 3671 | 3671 | UCAAAGUCUUUCUUUAGUU | 9161 | AACUAAAGAAAGACUUUGA |
| siRNA 3672 | 3672 | CAAAGUCUUUCUUUAGUUC | 9162 | GAACUAAAGAAAGACUUUG |
| siRNA 3673 | 3673 | AAAGUCUUUCUUUAGUUCA | 9163 | UGAACUAAAGAAAGACUUU |
| siRNA 3674 | 3674 | AAGUCUUUCUUUAGUUCAU | 9164 | AUGAACUAAAGAAAGACUU |
| siRNA 3675 | 3675 | AGUCUUUCUUUAGUUCAUG | 9165 | CAUGAACUAAAGAAAGACU |
| siRNA 3676 | 3676 | GUCUUUCUUUAGUUCAUGU | 9166 | ACAUGAACUAAAGAAAGAC |
| siRNA 3677 | 3677 | UCUUUCUUUAGUUCAUGUG | 9167 | CACAUGAACUAAAGAAAGA |
| siRNA 3678 | 3678 | CUUUCUUUAGUUCAUGUGG | 9168 | CCACAUGAACUAAAGAAAG |
| siRNA 3679 | 3679 | UUUCUUUAGUUCAUGUGGU | 9169 | ACCACAUGAACUAAAGAAA |
| siRNA 3680 | 3680 | UUCUUUAGUUCAUGUGGUA | 9170 | UACCACAUGAACUAAAGAA |
| siRNA 3681 | 3681 | UCUUUAGUUCAUGUGGUAG | 9171 | CUACCACAUGAACUAAAGA |
| siRNA 3682 | 3682 | CUUUAGUUCAUGUGGUAGU | 9172 | ACUACCACAUGAACUAAAG |
| siRNA 3683 | 3683 | UUUAGUUCAUGUGGUAGUU | 9173 | AACUACCACAUGAACUAAA |
| siRNA 3684 | 3684 | UUAGUUCAUGUGGUAGUUU | 9174 | AAACUACCACAUGAACUAA |
| siRNA 3685 | 3685 | UAGUUCAUGUGGUAGUUUA | 9175 | UAAACUACCACAUGAACUA |
| siRNA 3686 | 3686 | AGUUCAUGUGGUAGUUUAC | 9176 | GUAAACUACCACAUGAACU |
| siRNA 3687 | 3687 | GUUCAUGUGGUAGUUUACA | 9177 | UGUAAACUACCACAUGAAC |
| siRNA 3688 | 3688 | UUCAUGUGGUAGUUUACAU | 9178 | AUGUAAACUACCACAUGAA |
| siRNA 3689 | 3689 | UCAUGUGGUAGUUUACAUA | 9179 | UAUGUAAACUACCACAUGA |
| siRNA 3690 | 3690 | CAUGUGGUAGUUUACAUAA | 9180 | UUAUGUAAACUACCACAUG |
| siRNA 3691 | 3691 | AUGUGGUAGUUUACAUAAA | 9181 | UUUAUGUAAACUACCACAU |
| siRNA 3692 | 3692 | UGUGGUAGUUUACAUAAAC | 9182 | GUUUAUGUAAACUACCACA |
| siRNA 3693 | 3693 | GUGGUAGUUUACAUAAACU | 9183 | AGUUUAUGUAAACUACCAC |
| siRNA 3694 | 3694 | UGGUAGUUUACAUAAACUA | 9184 | UAGUUUAUGUAAACUACCA |
| siRNA 3695 | 3695 | GGUAGUUUACAUAAACUAA | 9185 | UUAGUUUAUGUAAACUACC |
| siRNA 3696 | 3696 | GUAGUUUACAUAAACUAAG | 9186 | CUUAGUUUAUGUAAACUAC |
| siRNA 3697 | 3697 | UAGUUUACAUAAACUAAGU | 9187 | ACUUAGUUUAUGUAAACUA |
| siRNA 3698 | 3698 | AGUUUACAUAAACUAAGUG | 9188 | CACUUAGUUUAUGUAAACU |
| siRNA 3699 | 3699 | GUUUACAUAAACUAAGUGA | 9189 | UCACUUAGUUUAUGUAAAC |
| siRNA 3700 | 3700 | UUUACAUAAACUAAGUGAA | 9190 | UUCACUUAGUUUAUGUAAA |
| siRNA 3701 | 3701 | UUACAUAAACUAAGUGAAG | 9191 | CUUCACUUAGUUUAUGUAA |
| siRNA 3702 | 3702 | UACAUAAACUAAGUGAAGA | 9192 | UCUUCACUUAGUUUAUGUA |
| siRNA 3703 | 3703 | ACAUAAACUAAGUGAAGAG | 9193 | CUCUUCACUUAGUUUAUGU |
| siRNA 3704 | 3704 | CAUAAACUAAGUGAAGAGC | 9194 | GCUCUUCACUUAGUUUAUG |
| siRNA 3705 | 3705 | AUAAACUAAGUGAAGAGCC | 9195 | GGCUCUUCACUUAGUUUAU |
| siRNA 3706 | 3706 | UAAACUAAGUGAAGAGCCC | 9196 | GGGCUCUUCACUUAGUUUA |
| siRNA 3707 | 3707 | AAACUAAGUGAAGAGCCCC | 9197 | GGGGCUCUUCACUUAGUUU |
| siRNA 3708 | 3708 | AACUAAGUGAAGAGCCCCU | 9198 | AGGGGCUCUUCACUUAGUU |
| siRNA 3709 | 3709 | ACUAAGUGAAGAGCCCCUG | 9199 | CAGGGGCUCUUCACUUAGU |
| siRNA 3710 | 3710 | CUAAGUGAAGAGCCCCUGA | 9200 | UCAGGGGCUCUUCACUUAG |
| siRNA 3711 | 3711 | UAAGUGAAGAGCCCCUGAU | 9201 | AUCAGGGGCUCUUCACUUA |
| siRNA 3712 | 3712 | AAGUGAAGAGCCCCUGAUU | 9202 | AAUCAGGGGCUCUUCACUU |
| siRNA 3713 | 3713 | AGUGAAGAGCCCCUGAUUC | 9203 | GAAUCAGGGGCUCUUCACU |
| siRNA 3714 | 3714 | GUGAAGAGCCCCUGAUUCC | 9204 | GGAAUCAGGGGCUCUUCAC |
| siRNA 3715 | 3715 | UGAAGAGCCCCUGAUUCCU | 9205 | AGGAAUCAGGGGCUCUUCA |
| siRNA 3716 | 3716 | GAAGAGCCCCUGAUUCCUC | 9206 | GAGGAAUCAGGGGCUCUUC |
| siRNA 3717 | 3717 | AAGAGCCCCUGAUUCCUCC | 9207 | GGAGGAAUCAGGGGCUCUU |
| siRNA 3718 | 3718 | AGAGCCCCUGAUUCCUCCU | 9208 | AGGAGGAAUCAGGGGCUCU |
| siRNA 3719 | 3719 | GAGCCCCUGAUUCCUCCUC | 9209 | GAGGAGGAAUCAGGGGCUC |
| siRNA 3720 | 3720 | AGCCCCUGAUUCCUCCUCC | 9210 | GGAGGAGGAAUCAGGGGCU |
| siRNA 3721 | 3721 | GCCCCUGAUUCCUCCUCCU | 9211 | AGGAGGAGGAAUCAGGGGC |
| siRNA 3722 | 3722 | CCCCUGAUUCCUCCUCCUC | 9212 | GAGGAGGAGGAAUCAGGGG |
| siRNA 3723 | 3723 | CCCUGAUUCCUCCUCCUCU | 9213 | AGAGGAGGAGGAAUCAGGG |
| siRNA 3724 | 3724 | CCUGAUUCCUCCUCCUCUU | 9214 | AAGAGGAGGAGGAAUCAGG |
| siRNA 3725 | 3725 | CUGAUUCCUCCUCCUCUUC | 9215 | GAAGAGGAGGAGGAAUCAG |
| siRNA 3726 | 3726 | UGAUUCCUCCUCCUCUUCC | 9216 | GGAAGAGGAGGAGGAAUCA |
| siRNA 3727 | 3727 | GAUUCCUCCUCCUCUUCCU | 9217 | AGGAAGAGGAGGAGGAAUC |
| siRNA 3728 | 3728 | AUUCCUCCUCCUCUUCCUC | 9218 | GAGGAAGAGGAGGAGGAAU |
| siRNA 3729 | 3729 | UUCCUCCUCCUCUUCCUCC | 9219 | GGAGGAAGAGGAGGAGGAA |
| siRNA 3730 | 3730 | UCCUCCUCCUCUUCCUCCU | 9220 | AGGAGGAAGAGGAGGAGGA |
| siRNA 3731 | 3731 | CCUCCUCCUCUUCCUCCUC | 9221 | GAGGAGGAAGAGGAGGAGG |
| siRNA 3732 | 3732 | CUCCUCCUCUUCCUCCUCG | 9222 | CGAGGAGGAAGAGGAGGAG |
| siRNA 3733 | 3733 | UCCUCCUCUUCCUCCUCGA | 9223 | UCGAGGAGGAAGAGGAGGA |
| siRNA 3734 | 3734 | CCUCCUCUUCCUCCUCGAA | 9224 | UUCGAGGAGGAAGAGGAGG |
| siRNA 3735 | 3735 | CUCCUCUUCCUCCUCGAAA | 9225 | UUUCGAGGAGGAAGAGGAG |
| siRNA 3736 | 3736 | UCCUCUUCCUCCUCGAAAA | 9226 | UUUUCGAGGAGGAAGAGGA |
| siRNA 3737 | 3737 | CCUCUUCCUCCUCGAAAAA | 9227 | UUUUUCGAGGAGGAAGAGG |
| siRNA 3738 | 3738 | CUCUUCCUCCUCGAAAAAA | 9228 | UUUUUUCGAGGAGGAAGAG |
| siRNA 3739 | 3739 | UCUUCCUCCUCGAAAAAAG | 9229 | CUUUUUUCGAGGAGGAAGA |
| siRNA 3740 | 3740 | CUUCCUCCUCGAAAAAAGU | 9230 | ACUUUUUUCGAGGAGGAAG |
| siRNA 3741 | 3741 | UUCCUCCUCGAAAAAAGUU | 9231 | AACUUUUUUCGAGGAGGAA |
| siRNA 3742 | 3742 | UCCUCCUCGAAAAAAGUUU | 9232 | AAACUUUUUUCGAGGAGGA |
| siRNA 3743 | 3743 | CCUCCUCGAAAAAAGUUUG | 9233 | CAAACUUUUUUCGAGGAGG |
| siRNA 3744 | 3744 | CUCCUCGAAAAAAGUUUGA | 9234 | UCAAACUUUUUUCGAGGAG |
| siRNA 3745 | 3745 | UCCUCGAAAAAAGUUUGAU | 9235 | AUCAAACUUUUUUCGAGGA |
| siRNA 3746 | 3746 | CCUCGAAAAAAGUUUGAUC | 9236 | GAUCAAACUUUUUUCGAGG |
| siRNA 3747 | 3747 | CUCGAAAAAAGUUUGAUCA | 9237 | UGAUCAAACUUUUUUCGAG |
| siRNA 3748 | 3748 | UCGAAAAAAGUUUGAUCAU | 9238 | AUGAUCAAACUUUUUUCGA |
| siRNA 3749 | 3749 | CGAAAAAAGUUUGAUCAUG | 9239 | CAUGAUCAAACUUUUUUCG |
| siRNA 3750 | 3750 | GAAAAAAGUUUGAUCAUGA | 9240 | UCAUGAUCAAACUUUUUUC |
| siRNA 3751 | 3751 | AAAAAAGUUUGAUCAUGAU | 9241 | AUCAUGAUCAAACUUUUUU |
| siRNA 3752 | 3752 | AAAAAGUUUGAUCAUGAUG | 9242 | CAUCAUGAUCAAACUUUUU |
| siRNA 3753 | 3753 | AAAAGUUUGAUCAUGAUGC | 9243 | GCAUCAUGAUCAAACUUUU |
| siRNA 3754 | 3754 | AAAGUUUGAUCAUGAUGCU | 9244 | AGCAUCAUGAUCAAACUUU |
| siRNA 3755 | 3755 | AAGUUUGAUCAUGAUGCUU | 9245 | AAGCAUCAUGAUCAAACUU |
| siRNA 3756 | 3756 | AGUUUGAUCAUGAUGCUUC | 9246 | GAAGCAUCAUGAUCAAACU |
| siRNA 3757 | 3757 | GUUUGAUCAUGAUGCUUCA | 9247 | UGAAGCAUCAUGAUCAAAC |
| siRNA 3758 | 3758 | UUUGAUCAUGAUGCUUCAA | 9248 | UUGAAGCAUCAUGAUCAAA |
| siRNA 3759 | 3759 | UUGAUCAUGAUGCUUCAAA | 9249 | UUUGAAGCAUCAUGAUCAA |
| siRNA 3760 | 3760 | UGAUCAUGAUGCUUCAAAU | 9250 | AUUUGAAGCAUCAUGAUCA |
| siRNA 3761 | 3761 | GAUCAUGAUGCUUCAAAUU | 9251 | AAUUUGAAGCAUCAUGAUC |
| siRNA 3762 | 3762 | AUCAUGAUGCUUCAAAUUC | 9252 | GAAUUUGAAGCAUCAUGAU |
| siRNA 3763 | 3763 | UCAUGAUGCUUCAAAUUCC | 9253 | GGAAUUUGAAGCAUCAUGA |
| siRNA 3764 | 3764 | CAUGAUGCUUCAAAUUCCA | 9254 | UGGAAUUUGAAGCAUCAUG |
| siRNA 3765 | 3765 | AUGAUGCUUCAAAUUCCAA | 9255 | UUGGAAUUUGAAGCAUCAU |
| siRNA 3766 | 3766 | UGAUGCUUCAAAUUCCAAG | 9256 | CUUGGAAUUUGAAGCAUCA |
| siRNA 3767 | 3767 | GAUGCUUCAAAUUCCAAGG | 9257 | CCUUGGAAUUUGAAGCAUC |
| siRNA 3768 | 3768 | AUGCUUCAAAUUCCAAGGG | 9258 | CCCUUGGAAUUUGAAGCAU |
| siRNA 3769 | 3769 | UGCUUCAAAUUCCAAGGGA | 9259 | UCCCUUGGAAUUUGAAGCA |
| siRNA 3770 | 3770 | GCUUCAAAUUCCAAGGGAA | 9260 | UUCCCUUGGAAUUUGAAGC |
| siRNA 3771 | 3771 | CUUCAAAUUCCAAGGGAAA | 9261 | UUUCCCUUGGAAUUUGAAG |
| siRNA 3772 | 3772 | UUCAAAUUCCAAGGGAAAU | 9262 | AUUUCCCUUGGAAUUUGAA |
| siRNA 3773 | 3773 | UCAAAUUCCAAGGGAAAUA | 9263 | UAUUUCCCUUGGAAUUUGA |
| siRNA 3774 | 3774 | CAAAUUCCAAGGGAAAUAU | 9264 | AUAUUUCCCUUGGAAUUUG |
| siRNA 3775 | 3775 | AAAUUCCAAGGGAAAUAUG | 9265 | CAUAUUUCCCUUGGAAUUU |
| siRNA 3776 | 3776 | AAUUCCAAGGGAAAUAUGA | 9266 | UCAUAUUUCCCUUGGAAUU |
| siRNA 3777 | 3777 | AUUCCAAGGGAAAUAUGAA | 9267 | UUCAUAUUUCCCUUGGAAU |
| siRNA 3778 | 3778 | UUCCAAGGGAAAUAUGAAA | 9268 | UUUCAUAUUUCCCUUGGAA |
| siRNA 3779 | 3779 | UCCAAGGGAAAUAUGAAAU | 9269 | AUUUCAUAUUUCCCUUGGA |
| siRNA 3780 | 3780 | CCAAGGGAAAUAUGAAAUC | 9270 | GAUUUCAUAUUUCCCUUGG |
| siRNA 3781 | 3781 | CAAGGGAAAUAUGAAAUCU | 9271 | AGAUUUCAUAUUUCCCUUG |
| siRNA 3782 | 3782 | AAGGGAAAUAUGAAAUCUG | 9272 | CAGAUUUCAUAUUUCCCUU |
| siRNA 3783 | 3783 | AGGGAAAUAUGAAAUCUGA | 9273 | UCAGAUUUCAUAUUUCCCU |
| siRNA 3784 | 3784 | GGGAAAUAUGAAAUCUGAU | 9274 | AUCAGAUUUCAUAUUUCCC |
| siRNA 3785 | 3785 | GGAAAUAUGAAAUCUGAUG | 9275 | CAUCAGAUUUCAUAUUUCC |
| siRNA 3786 | 3786 | GAAAUAUGAAAUCUGAUGA | 9276 | UCAUCAGAUUUCAUAUUUC |
| siRNA 3787 | 3787 | AAAUAUGAAAUCUGAUGAU | 9277 | AUCAUCAGAUUUCAUAUUU |
| siRNA 3788 | 3788 | AAUAUGAAAUCUGAUGAUG | 9278 | CAUCAUCAGAUUUCAUAUU |
| siRNA 3789 | 3789 | AUAUGAAAUCUGAUGAUGA | 9279 | UCAUCAUCAGAUUUCAUAU |
| siRNA 3790 | 3790 | UAUGAAAUCUGAUGAUGAU | 9280 | AUCAUCAUCAGAUUUCAUA |
| siRNA 3791 | 3791 | AUGAAAUCUGAUGAUGAUC | 9281 | GAUCAUCAUCAGAUUUCAU |
| siRNA 3792 | 3792 | UGAAAUCUGAUGAUGAUCC | 9282 | GGAUCAUCAUCAGAUUUCA |
| siRNA 3793 | 3793 | GAAAUCUGAUGAUGAUCCU | 9283 | AGGAUCAUCAUCAGAUUUC |
| siRNA 3794 | 3794 | AAAUCUGAUGAUGAUCCUC | 9284 | GAGGAUCAUCAUCAGAUUU |
| siRNA 3795 | 3795 | AAUCUGAUGAUGAUCCUCC | 9285 | GGAGGAUCAUCAUCAGAUU |
| siRNA 3796 | 3796 | AUCUGAUGAUGAUCCUCCU | 9286 | AGGAGGAUCAUCAUCAGAU |
| siRNA 3797 | 3797 | UCUGAUGAUGAUCCUCCUG | 9287 | CAGGAGGAUCAUCAUCAGA |
| siRNA 3798 | 3798 | CUGAUGAUGAUCCUCCUGC | 9288 | GCAGGAGGAUCAUCAUCAG |
| siRNA 3799 | 3799 | UGAUGAUGAUCCUCCUGCU | 9289 | AGCAGGAGGAUCAUCAUCA |
| siRNA 3800 | 3800 | GAUGAUGAUCCUCCUGCUA | 9290 | UAGCAGGAGGAUCAUCAUC |
| siRNA 3801 | 3801 | AUGAUGAUCCUCCUGCUAU | 9291 | AUAGCAGGAGGAUCAUCAU |
| siRNA 3802 | 3802 | UGAUGAUCCUCCUGCUAUU | 9292 | AAUAGCAGGAGGAUCAUCA |
| siRNA 3803 | 3803 | GAUGAUCCUCCUGCUAUUC | 9293 | GAAUAGCAGGAGGAUCAUC |
| siRNA 3804 | 3804 | AUGAUCCUCCUGCUAUUCC | 9294 | GGAAUAGCAGGAGGAUCAU |
| siRNA 3805 | 3805 | UGAUCCUCCUGCUAUUCCA | 9295 | UGGAAUAGCAGGAGGAUCA |
| siRNA 3806 | 3806 | GAUCCUCCUGCUAUUCCAC | 9296 | GUGGAAUAGCAGGAGGAUC |
| siRNA 3807 | 3807 | AUCCUCCUGCUAUUCCACC | 9297 | GGUGGAAUAGCAGGAGGAU |
| siRNA 3808 | 3808 | UCCUCCUGCUAUUCCACCG | 9298 | CGGUGGAAUAGCAGGAGGA |
| siRNA 3809 | 3809 | CCUCCUGCUAUUCCACCGA | 9299 | UCGGUGGAAUAGCAGGAGG |
| siRNA 3810 | 3810 | CUCCUGCUAUUCCACCGAG | 9300 | CUCGGUGGAAUAGCAGGAG |
| siRNA 3811 | 3811 | UCCUGCUAUUCCACCGAGA | 9301 | UCUCGGUGGAAUAGCAGGA |
| siRNA 3812 | 3812 | CCUGCUAUUCCACCGAGAC | 9302 | GUCUCGGUGGAAUAGCAGG |
| siRNA 3813 | 3813 | CUGCUAUUCCACCGAGACA | 9303 | UGUCUCGGUGGAAUAGCAG |
| siRNA 3814 | 3814 | UGCUAUUCCACCGAGACAG | 9304 | CUGUCUCGGUGGAAUAGCA |
| siRNA 3815 | 3815 | GCUAUUCCACCGAGACAGC | 9305 | GCUGUCUCGGUGGAAUAGC |
| siRNA 3816 | 3816 | CUAUUCCACCGAGACAGCC | 9306 | GGCUGUCUCGGUGGAAUAG |
| siRNA 3817 | 3817 | UAUUCCACCGAGACAGCCU | 9307 | AGGCUGUCUCGGUGGAAUA |
| siRNA 3818 | 3818 | AUUCCACCGAGACAGCCUC | 9308 | GAGGCUGUCUCGGUGGAAU |
| siRNA 3819 | 3819 | UUCCACCGAGACAGCCUCC | 9309 | GGAGGCUGUCUCGGUGGAA |
| siRNA 3820 | 3820 | UCCACCGAGACAGCCUCCU | 9310 | AGGAGGCUGUCUCGGUGGA |
| siRNA 3821 | 3821 | CCACCGAGACAGCCUCCUC | 9311 | GAGGAGGCUGUCUCGGUGG |
| siRNA 3822 | 3822 | CACCGAGACAGCCUCCUCC | 9312 | GGAGGAGGCUGUCUCGGUG |
| siRNA 3823 | 3823 | ACCGAGACAGCCUCCUCCU | 9313 | AGGAGGAGGCUGUCUCGGU |
| siRNA 3824 | 3824 | CCGAGACAGCCUCCUCCUC | 9314 | GAGGAGGAGGCUGUCUCGG |
| siRNA 3825 | 3825 | CGAGACAGCCUCCUCCUCC | 9315 | GGAGGAGGAGGCUGUCUCG |
| siRNA 3826 | 3826 | GAGACAGCCUCCUCCUCCA | 9316 | UGGAGGAGGAGGCUGUCUC |
| siRNA 3827 | 3827 | AGACAGCCUCCUCCUCCAA | 9317 | UUGGAGGAGGAGGCUGUCU |
| siRNA 3828 | 3828 | GACAGCCUCCUCCUCCAAA | 9318 | UUUGGAGGAGGAGGCUGUC |
| siRNA 3829 | 3829 | ACAGCCUCCUCCUCCAAAG | 9319 | CUUUGGAGGAGGAGGCUGU |
| siRNA 3830 | 3830 | CAGCCUCCUCCUCCAAAGG | 9320 | CCUUUGGAGGAGGAGGCUG |
| siRNA 3831 | 3831 | AGCCUCCUCCUCCAAAGGU | 9321 | ACCUUUGGAGGAGGAGGCU |
| siRNA 3832 | 3832 | GCCUCCUCCUCCAAAGGUA | 9322 | UACCUUUGGAGGAGGAGGC |
| siRNA 3833 | 3833 | CCUCCUCCUCCAAAGGUAA | 9323 | UUACCUUUGGAGGAGGAGG |
| siRNA 3834 | 3834 | CUCCUCCUCCAAAGGUAAA | 9324 | UUUACCUUUGGAGGAGGAG |
| siRNA 3835 | 3835 | UCCUCCUCCAAAGGUAAAA | 9325 | UUUUACCUUUGGAGGAGGA |
| siRNA 3836 | 3836 | CCUCCUCCAAAGGUAAAAC | 9326 | GUUUUACCUUUGGAGGAGG |
| siRNA 3837 | 3837 | CUCCUCCAAAGGUAAAACC | 9327 | GGUUUUACCUUUGGAGGAG |
| siRNA 3838 | 3838 | UCCUCCAAAGGUAAAACCC | 9328 | GGGUUUUACCUUUGGAGGA |
| siRNA 3839 | 3839 | CCUCCAAAGGUAAAACCCA | 9329 | UGGGUUUUACCUUUGGAGG |
| siRNA 3840 | 3840 | CUCCAAAGGUAAAACCCAG | 9330 | CUGGGUUUUACCUUUGGAG |
| siRNA 3841 | 3841 | UCCAAAGGUAAAACCCAGA | 9331 | UCUGGGUUUUACCUUUGGA |
| siRNA 3842 | 3842 | CCAAAGGUAAAACCCAGAG | 9332 | CUCUGGGUUUUACCUUUGG |
| siRNA 3843 | 3843 | CAAAGGUAAAACCCAGAGU | 9333 | ACUCUGGGUUUUACCUUUG |
| siRNA 3844 | 3844 | AAAGGUAAAACCCAGAGUU | 9334 | AACUCUGGGUUUUACCUUU |
| siRNA 3845 | 3845 | AAGGUAAAACCCAGAGUUC | 9335 | GAACUCUGGGUUUUACCUU |
| siRNA 3846 | 3846 | AGGUAAAACCCAGAGUUCC | 9336 | GGAACUCUGGGUUUUACCU |
| siRNA 3847 | 3847 | GGUAAAACCCAGAGUUCCU | 9337 | AGGAACUCUGGGUUUUACC |
| siRNA 3848 | 3848 | GUAAAACCCAGAGUUCCUG | 9338 | CAGGAACUCUGGGUUUUAC |
| siRNA 3849 | 3849 | UAAAACCCAGAGUUCCUGU | 9339 | ACAGGAACUCUGGGUUUUA |
| siRNA 3850 | 3850 | AAAACCCAGAGUUCCUGUU | 9340 | AACAGGAACUCUGGGUUUU |
| siRNA 3851 | 3851 | AAACCCAGAGUUCCUGUUC | 9341 | GAACAGGAACUCUGGGUUU |
| siRNA 3852 | 3852 | AACCCAGAGUUCCUGUUCC | 9342 | GGAACAGGAACUCUGGGUU |
| siRNA 3853 | 3853 | ACCCAGAGUUCCUGUUCCU | 9343 | AGGAACAGGAACUCUGGGU |
| siRNA 3854 | 3854 | CCCAGAGUUCCUGUUCCUA | 9344 | UAGGAACAGGAACUCUGGG |
| siRNA 3855 | 3855 | CCAGAGUUCCUGUUCCUAC | 9345 | GUAGGAACAGGAACUCUGG |
| siRNA 3856 | 3856 | CAGAGUUCCUGUUCCUACU | 9346 | AGUAGGAACAGGAACUCUG |
| siRNA 3857 | 3857 | AGAGUUCCUGUUCCUACUG | 9347 | CAGUAGGAACAGGAACUCU |
| siRNA 3858 | 3858 | GAGUUCCUGUUCCUACUGG | 9348 | CCAGUAGGAACAGGAACUC |
| siRNA 3859 | 3859 | AGUUCCUGUUCCUACUGGU | 9349 | ACCAGUAGGAACAGGAACU |
| siRNA 3860 | 3860 | GUUCCUGUUCCUACUGGUG | 9350 | CACCAGUAGGAACAGGAAC |
| siRNA 3861 | 3861 | UUCCUGUUCCUACUGGUGC | 9351 | GCACCAGUAGGAACAGGAA |
| siRNA 3862 | 3862 | UCCUGUUCCUACUGGUGCA | 9352 | UGCACCAGUAGGAACAGGA |
| siRNA 3863 | 3863 | CCUGUUCCUACUGGUGCAU | 9353 | AUGCACCAGUAGGAACAGG |
| siRNA 3864 | 3864 | CUGUUCCUACUGGUGCAUU | 9354 | AAUGCACCAGUAGGAACAG |
| siRNA 3865 | 3865 | UGUUCCUACUGGUGCAUUU | 9355 | AAAUGCACCAGUAGGAACA |
| siRNA 3866 | 3866 | GUUCCUACUGGUGCAUUUG | 9356 | CAAAUGCACCAGUAGGAAC |
| siRNA 3867 | 3867 | UUCCUACUGGUGCAUUUGA | 9357 | UCAAAUGCACCAGUAGGAA |
| siRNA 3868 | 3868 | UCCUACUGGUGCAUUUGAU | 9358 | AUCAAAUGCACCAGUAGGA |
| siRNA 3869 | 3869 | CCUACUGGUGCAUUUGAUG | 9359 | CAUCAAAUGCACCAGUAGG |
| siRNA 3870 | 3870 | CUACUGGUGCAUUUGAUGG | 9360 | CCAUCAAAUGCACCAGUAG |
| siRNA 3871 | 3871 | UACUGGUGCAUUUGAUGGG | 9361 | CCCAUCAAAUGCACCAGUA |
| siRNA 3872 | 3872 | ACUGGUGCAUUUGAUGGGC | 9362 | GCCCAUCAAAUGCACCAGU |
| siRNA 3873 | 3873 | CUGGUGCAUUUGAUGGGCC | 9363 | GGCCCAUCAAAUGCACCAG |
| siRNA 3874 | 3874 | UGGUGCAUUUGAUGGGCCU | 9364 | AGGCCCAUCAAAUGCACCA |
| siRNA 3875 | 3875 | GGUGCAUUUGAUGGGCCUC | 9365 | GAGGCCCAUCAAAUGCACC |
| siRNA 3876 | 3876 | GUGCAUUUGAUGGGCCUCU | 9366 | AGAGGCCCAUCAAAUGCAC |
| siRNA 3877 | 3877 | UGCAUUUGAUGGGCCUCUG | 9367 | CAGAGGCCCAUCAAAUGCA |
| siRNA 3878 | 3878 | GCAUUUGAUGGGCCUCUGC | 9368 | GCAGAGGCCCAUCAAAUGC |
| siRNA 3879 | 3879 | CAUUUGAUGGGCCUCUGCA | 9369 | UGCAGAGGCCCAUCAAAUG |
| siRNA 3880 | 3880 | AUUUGAUGGGCCUCUGCAU | 9370 | AUGCAGAGGCCCAUCAAAU |
| siRNA 3881 | 388 | UUUGAUGGGCCUCUGCAUA | 9371 | UAUGCAGAGGCCCAUCAAA |
| siRNA 3882 | 3882 | UUGAUGGGCCUCUGCAUAG | 9372 | CUAUGCAGAGGCCCAUCAA |
| siRNA 3883 | 3883 | UGAUGGGCCUCUGCAUAGU | 9373 | ACUAUGCAGAGGCCCAUCA |
| siRNA 3884 | 3884 | GAUGGGCCUCUGCAUAGUC | 9374 | GACUAUGCAGAGGCCCAUC |
| siRNA 3885 | 3885 | AUGGGCCUCUGCAUAGUCC | 9375 | GGACUAUGCAGAGGCCCAU |
| siRNA 3886 | 3886 | UGGGCCUCUGCAUAGUCCA | 9376 | UGGACUAUGCAGAGGCCCA |
| siRNA 3887 | 3887 | GGGCCUCUGCAUAGUCCAC | 9377 | GUGGACUAUGCAGAGGCCC |
| siRNA 3888 | 3888 | GGCCUCUGCAUAGUCCACC | 9378 | GGUGGACUAUGCAGAGGCC |
| siRNA 3889 | 3889 | GCCUCUGCAUAGUCCACCU | 9379 | AGGUGGACUAUGCAGAGGC |
| siRNA 3890 | 3890 | CCUCUGCAUAGUCCACCUC | 9380 | GAGGUGGACUAUGCAGAGG |
| siRNA 3891 | 3891 | CUCUGCAUAGUCCACCUCC | 9381 | GGAGGUGGACUAUGCAGAG |
| siRNA 3892 | 3892 | UCUGCAUAGUCCACCUCCG | 9382 | CGGAGGUGGACUAUGCAGA |
| siRNA 3893 | 3893 | CUGCAUAGUCCACCUCCGC | 9383 | GCGGAGGUGGACUAUGCAG |
| siRNA 3894 | 3894 | UGCAUAGUCCACCUCCGCC | 9384 | GGCGGAGGUGGACUAUGCA |
| siRNA 3895 | 3895 | GCAUAGUCCACCUCCGCCA | 9385 | UGGCGGAGGUGGACUAUGC |
| siRNA 3896 | 3896 | CAUAGUCCACCUCCGCCAC | 9386 | GUGGCGGAGGUGGACUAUG |
| siRNA 3897 | 3897 | AUAGUCCACCUCCGCCACC | 9387 | GGUGGCGGAGGUGGACUAU |
| siRNA 3898 | 3898 | UAGUCCACCUCCGCCACCA | 9388 | UGGUGGCGGAGGUGGACUA |
| siRNA 3899 | 3899 | AGUCCACCUCCGCCACCAC | 9389 | GUGGUGGCGGAGGUGGACU |
| siRNA 3900 | 3900 | GUCCACCUCCGCCACCACC | 9390 | GGUGGUGGCGGAGGUGGAC |
| siRNA 3901 | 3901 | UCCACCUCCGCCACCACCA | 9391 | UGGUGGUGGCGGAGGUGGA |
| siRNA 3902 | 3902 | CCACCUCCGCCACCACCAA | 9392 | UUGGUGGUGGCGGAGGUGG |
| siRNA 3903 | 3903 | CACCUCCGCCACCACCAAG | 9393 | CUUGGUGGUGGCGGAGGUG |
| siRNA 3904 | 3904 | ACCUCCGCCACCACCAAGA | 9394 | UCUUGGUGGUGGCGGAGGU |
| siRNA 3905 | 3905 | CCUCCGCCACCACCAAGAG | 9395 | CUCUUGGUGGUGGCGGAGG |
| siRNA 3906 | 3906 | CUCCGCCACCACCAAGAGA | 9396 | UCUCUUGGUGGUGGCGGAG |
| siRNA 3907 | 3907 | UCCGCCACCACCAAGAGAU | 9397 | AUCUCUUGGUGGUGGCGGA |
| siRNA 3908 | 3908 | CCGCCACCACCAAGAGAUC | 9398 | GAUCUCUUGGUGGUGGCGG |
| siRNA 3909 | 3909 | CGCCACCACCAAGAGAUCC | 9399 | GGAUCUCUUGGUGGUGGCG |
| siRNA 3910 | 3910 | GCCACCACCAAGAGAUCCU | 9400 | AGGAUCUCUUGGUGGUGGC |
| siRNA 3911 | 3911 | CCACCACCAAGAGAUCCUC | 9401 | GAGGAUCUCUUGGUGGUGG |
| siRNA 3912 | 3912 | CACCACCAAGAGAUCCUCU | 9402 | AGAGGAUCUCUUGGUGGUG |
| siRNA 3913 | 3913 | ACCACCAAGAGAUCCUCUU | 9403 | AAGAGGAUCUCUUGGUGGU |
| siRNA 3914 | 3914 | CCACCAAGAGAUCCUCUUC | 9404 | GAAGAGGAUCUCUUGGUGG |
| siRNA 3915 | 3915 | CACCAAGAGAUCCUCUUCC | 9405 | GGAAGAGGAUCUCUUGGUG |
| siRNA 3916 | 3916 | ACCAAGAGAUCCUCUUCCU | 9406 | AGGAAGAGGAUCUCUUGGU |
| siRNA 3917 | 3917 | CCAAGAGAUCCUCUUCCUG | 9407 | CAGGAAGAGGAUCUCUUGG |
| siRNA 3918 | 3918 | CAAGAGAUCCUCUUCCUGA | 9408 | UCAGGAAGAGGAUCUCUUG |
| siRNA 3919 | 3919 | AAGAGAUCCUCUUCCUGAU | 9409 | AUCAGGAAGAGGAUCUCUU |
| siRNA 3920 | 3920 | AGAGAUCCUCUUCCUGAUA | 9410 | UAUCAGGAAGAGGAUCUCU |
| siRNA 3921 | 3921 | GAGAUCCUCUUCCUGAUAC | 9411 | GUAUCAGGAAGAGGAUCUC |
| siRNA 3922 | 3922 | AGAUCCUCUUCCUGAUACC | 9412 | GGUAUCAGGAAGAGGAUCU |
| siRNA 3923 | 3923 | GAUCCUCUUCCUGAUACCC | 9413 | GGGUAUCAGGAAGAGGAUC |
| siRNA 3924 | 3924 | AUCCUCUUCCUGAUACCCC | 9414 | GGGGUAUCAGGAAGAGGAU |
| siRNA 3925 | 3925 | UCCUCUUCCUGAUACCCCU | 9415 | AGGGGUAUCAGGAAGAGGA |
| siRNA 3926 | 3926 | CCUCUUCCUGAUACCCCUC | 9416 | GAGGGGUAUCAGGAAGAGG |
| siRNA 3927 | 3927 | CUCUUCCUGAUACCCCUCC | 9417 | GGAGGGGUAUCAGGAAGAG |
| siRNA 3928 | 3928 | UCUUCCUGAUACCCCUCCA | 9418 | UGGAGGGGUAUCAGGAAGA |
| siRNA 3929 | 3929 | CUUCCUGAUACCCCUCCAC | 9419 | GUGGAGGGGUAUCAGGAAG |
| siRNA 3930 | 3930 | UUCCUGAUACCCCUCCACC | 9420 | GGUGGAGGGGUAUCAGGAA |
| siRNA 3931 | 3931 | UCCUGAUACCCCUCCACCA | 9421 | UGGUGGAGGGGUAUCAGGA |
| siRNA 3932 | 3932 | CCUGAUACCCCUCCACCAG | 9422 | CUGGUGGAGGGGUAUCAGG |
| siRNA 3933 | 3933 | CUGAUACCCCUCCACCAGU | 9423 | ACUGGUGGAGGGGUAUCAG |
| siRNA 3934 | 3934 | UGAUACCCCUCCACCAGUU | 9424 | AACUGGUGGAGGGGUAUCA |
| siRNA 3935 | 3935 | GAUACCCCUCCACCAGUUC | 9425 | GAACUGGUGGAGGGGUAUC |
| siRNA 3936 | 3936 | AUACCCCUCCACCAGUUCC | 9426 | GGAACUGGUGGAGGGGUAU |
| siRNA 3937 | 3937 | UACCCCUCCACCAGUUCCC | 9427 | GGGAACUGGUGGAGGGGUA |
| siRNA 3938 | 3938 | ACCCCUCCACCAGUUCCCC | 9428 | GGGGAACUGGUGGAGGGGU |
| siRNA 3939 | 3939 | CCCCUCCACCAGUUCCCCU | 9429 | AGGGGAACUGGUGGAGGGG |
| siRNA 3940 | 3940 | CCCUCCACCAGUUCCCCUU | 9430 | AAGGGGAACUGGUGGAGGG |
| siRNA 3941 | 3941 | CCUCCACCAGUUCCCCUUC | 9431 | GAAGGGGAACUGGUGGAGG |
| siRNA 3942 | 3942 | CUCCACCAGUUCCCCUUCG | 9432 | CGAAGGGGAACUGGUGGAG |
| siRNA 3943 | 3943 | UCCACCAGUUCCCCUUCGG | 9433 | CCGAAGGGGAACUGGUGGA |
| siRNA 3944 | 3944 | CCACCAGUUCCCCUUCGGC | 9434 | GCCGAAGGGGAACUGGUGG |
| siRNA 3945 | 3945 | CACCAGUUCCCCUUCGGCC | 9435 | GGCCGAAGGGGAACUGGUG |
| siRNA 3946 | 3946 | ACCAGUUCCCCUUCGGCCU | 9436 | AGGCCGAAGGGGAACUGGU |
| siRNA 3947 | 3947 | CCAGUUCCCCUUCGGCCUC | 9437 | GAGGCCGAAGGGGAACUGG |
| siRNA 3948 | 3948 | CAGUUCCCCUUCGGCCUCC | 9438 | GGAGGCCGAAGGGGAACUG |
| siRNA 3949 | 3949 | AGUUCCCCUUCGGCCUCCA | 9439 | UGGAGGCCGAAGGGGAACU |
| siRNA 3950 | 3950 | GUUCCCCUUCGGCCUCCAG | 9440 | CUGGAGGCCGAAGGGGAAC |
| siRNA 3951 | 3951 | UUCCCCUUCGGCCUCCAGA | 9441 | UCUGGAGGCCGAAGGGGAA |
| siRNA 3952 | 3952 | UCCCCUUCGGCCUCCAGAA | 9442 | UUCUGGAGGCCGAAGGGGA |
| siRNA 3953 | 3953 | CCCCUUCGGCCUCCAGAAC | 9443 | GUUCUGGAGGCCGAAGGGG |
| siRNA 3954 | 3954 | CCCUUCGGCCUCCAGAACA | 9444 | UGUUCUGGAGGCCGAAGGG |
| siRNA 3955 | 3955 | CCUUCGGCCUCCAGAACAC | 9445 | GUGUUCUGGAGGCCGAAGG |
| siRNA 3956 | 3956 | CUUCGGCCUCCAGAACACU | 9446 | AGUGUUCUGGAGGCCGAAG |
| siRNA 3957 | 3957 | UUCGGCCUCCAGAACACUU | 9447 | AAGUGUUCUGGAGGCCGAA |
| siRNA 3958 | 3958 | UCGGCCUCCAGAACACUUU | 9448 | AAAGUGUUCUGGAGGCCGA |
| siRNA 3959 | 3959 | CGGCCUCCAGAACACUUUA | 9449 | UAAAGUGUUCUGGAGGCCG |
| siRNA 3960 | 3960 | GGCCUCCAGAACACUUUAU | 9450 | AUAAAGUGUUCUGGAGGCC |
| siRNA 3961 | 3961 | GCCUCCAGAACACUUUAUA | 9451 | UAUAAAGUGUUCUGGAGGC |
| siRNA 3962 | 3962 | CCUCCAGAACACUUUAUAA | 9452 | UUAUAAAGUGUUCUGGAGG |
| siRNA 3963 | 3963 | CUCCAGAACACUUUAUAAA | 9453 | UUUAUAAAGUGUUCUGGAG |
| siRNA 3964 | 3964 | UCCAGAACACUUUAUAAAC | 9454 | GUUUAUAAAGUGUUCUGGA |
| siRNA 3965 | 3965 | CCAGAACACUUUAUAAACU | 9455 | AGUUUAUAAAGUGUUCUGG |
| siRNA 3966 | 3966 | CAGAACACUUUAUAAACUG | 9456 | CAGUUUAUAAAGUGUUCUG |
| siRNA 3967 | 3967 | AGAACACUUUAUAAACUGU | 9457 | ACAGUUUAUAAAGUGUUCU |
| siRNA 3968 | 3968 | GAACACUUUAUAAACUGUC | 9458 | GACAGUUUAUAAAGUGUUC |
| siRNA 3969 | 3969 | AACACUUUAUAAACUGUCC | 9459 | GGACAGUUUAUAAAGUGUU |
| siRNA 3970 | 3970 | ACACUUUAUAAACUGUCCA | 9460 | UGGACAGUUUAUAAAGUGU |
| siRNA 3971 | 3971 | CACUUUAUAAACUGUCCAU | 9461 | AUGGACAGUUUAUAAAGUG |
| siRNA 3972 | 3972 | ACUUUAUAAACUGUCCAUU | 9462 | AAUGGACAGUUUAUAAAGU |
| siRNA 3973 | 3973 | CUUUAUAAACUGUCCAUUU | 9463 | AAAUGGACAGUUUAUAAAG |
| siRNA 3974 | 3974 | UUUAUAAACUGUCCAUUUA | 9464 | UAAAUGGACAGUUUAUAAA |
| siRNA 3975 | 3975 | UUAUAAACUGUCCAUUUAA | 9465 | UUAAAUGGACAGUUUAUAA |
| siRNA 3976 | 3976 | UAUAAACUGUCCAUUUAAU | 9466 | AUUAAAUGGACAGUUUAUA |
| siRNA 3977 | 3977 | AUAAACUGUCCAUUUAAUC | 9467 | GAUUAAAUGGACAGUUUAU |
| siRNA 3978 | 3978 | UAAACUGUCCAUUUAAUCU | 9468 | AGAUUAAAUGGACAGUUUA |
| siRNA 3979 | 3979 | AAACUGUCCAUUUAAUCUU | 9469 | AAGAUUAAAUGGACAGUUU |
| siRNA 3980 | 3980 | AACUGUCCAUUUAAUCUUC | 9470 | GAAGAUUAAAUGGACAGUU |
| siRNA 3981 | 3981 | ACUGUCCAUUUAAUCUUCA | 9471 | UGAAGAUUAAAUGGACAGU |
| siRNA 3982 | 3982 | CUGUCCAUUUAAUCUUCAG | 9472 | CUGAAGAUUAAAUGGACAG |
| siRNA 3983 | 3983 | UGUCCAUUUAAUCUUCAGC | 9473 | GCUGAAGAUUAAAUGGACA |
| siRNA 3984 | 3984 | GUCCAUUUAAUCUUCAGCC | 9474 | GGCUGAAGAUUAAAUGGAC |
| siRNA 3985 | 3985 | UCCAUUUAAUCUUCAGCCA | 9475 | UGGCUGAAGAUUAAAUGGA |
| siRNA 3986 | 3986 | CCAUUUAAUCUUCAGCCAC | 9476 | GUGGCUGAAGAUUAAAUGG |
| siRNA 3987 | 3987 | CAUUUAAUCUUCAGCCACC | 9477 | GGUGGCUGAAGAUUAAAUG |
| siRNA 3988 | 3988 | AUUUAAUCUUCAGCCACCU | 9478 | AGGUGGCUGAAGAUUAAAU |
| siRNA 3989 | 3989 | UUUAAUCUUCAGCCACCUC | 9479 | GAGGUGGCUGAAGAUUAAA |
| siRNA 3990 | 3990 | UUAAUCUUCAGCCACCUCC | 9480 | GGAGGUGGCUGAAGAUUAA |
| siRNA 3991 | 3991 | UAAUCUUCAGCCACCUCCA | 9481 | UGGAGGUGGCUGAAGAUUA |
| siRNA 3992 | 3992 | AAUCUUCAGCCACCUCCAC | 9482 | GUGGAGGUGGCUGAAGAUU |
| siRNA 3993 | 3993 | AUCUUCAGCCACCUCCACU | 9483 | AGUGGAGGUGGCUGAAGAU |
| siRNA 3994 | 3994 | UCUUCAGCCACCUCCACUG | 9484 | CAGUGGAGGUGGCUGAAGA |
| siRNA 3995 | 3995 | CUUCAGCCACCUCCACUGG | 9485 | CCAGUGGAGGUGGCUGAAG |
| siRNA 3996 | 3996 | UUCAGCCACCUCCACUGGG | 9486 | CCCAGUGGAGGUGGCUGAA |
| siRNA 3997 | 3997 | UCAGCCACCUCCACUGGGG | 9487 | CCCCAGUGGAGGUGGCUGA |
| siRNA 3998 | 3998 | CAGCCACCUCCACUGGGGC | 9488 | GCCCCAGUGGAGGUGGCUG |
| siRNA 3999 | 3999 | AGCCACCUCCACUGGGGCA | 9489 | UGCCCCAGUGGAGGUGGCU |
| siRNA 4000 | 4000 | GCCACCUCCACUGGGGCAU | 9490 | AUGCCCCAGUGGAGGUGGC |
| siRNA 4001 | 4001 | CCACCUCCACUGGGGCAUC | 9491 | GAUGCCCCAGUGGAGGUGG |
| siRNA 4002 | 4002 | CACCUCCACUGGGGCAUCU | 9492 | AGAUGCCCCAGUGGAGGUG |
| siRNA 4003 | 4003 | ACCUCCACUGGGGCAUCUU | 9493 | AAGAUGCCCCAGUGGAGGU |
| siRNA 4004 | 4004 | CCUCCACUGGGGCAUCUUC | 9494 | GAAGAUGCCCCAGUGGAGG |
| siRNA 4005 | 4005 | CUCCACUGGGGCAUCUUCA | 9495 | UGAAGAUGCCCCAGUGGAG |
| siRNA 4006 | 4006 | UCCACUGGGGCAUCUUCAC | 9496 | GUGAAGAUGCCCCAGUGGA |
| siRNA 4007 | 4007 | CCACUGGGGCAUCUUCACA | 9497 | UGUGAAGAUGCCCCAGUGG |
| siRNA 4008 | 4008 | CACUGGGGCAUCUUCACAG | 9498 | CUGUGAAGAUGCCCCAGUG |
| siRNA 4009 | 4009 | ACUGGGGCAUCUUCACAGA | 9499 | UCUGUGAAGAUGCCCCAGU |
| siRNA 4010 | 4010 | CUGGGGCAUCUUCACAGAG | 9500 | CUCUGUGAAGAUGCCCCAG |
| siRNA 4011 | 4011 | UGGGGCAUCUUCACAGAGA | 9501 | UCUCUGUGAAGAUGCCCCA |
| siRNA 4012 | 4012 | GGGGCAUCUUCACAGAGAU | 9502 | AUCUCUGUGAAGAUGCCCC |
| siRNA 4013 | 4013 | GGGCAUCUUCACAGAGAUU | 9503 | AAUCUCUGUGAAGAUGCCC |
| siRNA 4014 | 4014 | GGCAUCUUCACAGAGAUUC | 9504 | GAAUCUCUGUGAAGAUGCC |
| siRNA 4015 | 4015 | GCAUCUUCACAGAGAUUCA | 9505 | UGAAUCUCUGUGAAGAUGC |
| siRNA 4016 | 4016 | CAUCUUCACAGAGAUUCAG | 9506 | CUGAAUCUCUGUGAAGAUG |
| siRNA 4017 | 4017 | AUCUUCACAGAGAUUCAGA | 9507 | UCUGAAUCUCUGUGAAGAU |
| siRNA 4018 | 4018 | UCUUCACAGAGAUUCAGAC | 9508 | GUCUGAAUCUCUGUGAAGA |
| siRNA 4019 | 4019 | CUUCACAGAGAUUCAGACU | 9509 | AGUCUGAAUCUCUGUGAAG |
| siRNA 4020 | 4020 | UUCACAGAGAUUCAGACUG | 9510 | CAGUCUGAAUCUCUGUGAA |
| siRNA 4021 | 4021 | UCACAGAGAUUCAGACUGG | 9511 | CCAGUCUGAAUCUCUGUGA |
| siRNA 4022 | 4022 | CACAGAGAUUCAGACUGGC | 9512 | GCCAGUCUGAAUCUCUGUG |
| siRNA 4023 | 4023 | ACAGAGAUUCAGACUGGCU | 9513 | AGCCAGUCUGAAUCUCUGU |
| siRNA 4024 | 4024 | CAGAGAUUCAGACUGGCUC | 9514 | GAGCCAGUCUGAAUCUCUG |
| siRNA 4025 | 4025 | AGAGAUUCAGACUGGCUCA | 9515 | UGAGCCAGUCUGAAUCUCU |
| siRNA 4026 | 4026 | GAGAUUCAGACUGGCUCAG | 9516 | CUGAGCCAGUCUGAAUCUC |
| siRNA 4027 | 4027 | AGAUUCAGACUGGCUCAGA | 9517 | UCUGAGCCAGUCUGAAUCU |
| siRNA 4028 | 4028 | GAUUCAGACUGGCUCAGAG | 9518 | CUCUGAGCCAGUCUGAAUC |
| siRNA 4029 | 4029 | AUUCAGACUGGCUCAGAGA | 9519 | UCUCUGAGCCAGUCUGAAU |
| siRNA 4030 | 4030 | UUCAGACUGGCUCAGAGAC | 9520 | GUCUCUGAGCCAGUCUGAA |
| siRNA 4031 | 4031 | UCAGACUGGCUCAGAGACA | 9521 | UGUCUCUGAGCCAGUCUGA |
| siRNA 4032 | 4032 | CAGACUGGCUCAGAGACAU | 9522 | AUGUCUCUGAGCCAGUCUG |
| siRNA 4033 | 4033 | AGACUGGCUCAGAGACAUU | 9523 | AAUGUCUCUGAGCCAGUCU |
| siRNA 4034 | 4034 | GACUGGCUCAGAGACAUUA | 9524 | UAAUGUCUCUGAGCCAGUC |
| siRNA 4035 | 4035 | ACUGGCUCAGAGACAUUAG | 9525 | CUAAUGUCUCUGAGCCAGU |
| siRNA 4036 | 4036 | CUGGCUCAGAGACAUUAGU | 9526 | ACUAAUGUCUCUGAGCCAG |
| siRNA 4037 | 4037 | UGGCUCAGAGACAUUAGUA | 9527 | UACUAAUGUCUCUGAGCCA |
| siRNA 4038 | 4038 | GGCUCAGAGACAUUAGUAC | 9528 | GUACUAAUGUCUCUGAGCC |
| siRNA 4039 | 4039 | GCUCAGAGACAUUAGUACG | 9529 | CGUACUAAUGUCUCUGAGC |
| siRNA 4040 | 4040 | CUCAGAGACAUUAGUACGU | 9530 | ACGUACUAAUGUCUCUGAG |
| siRNA 4041 | 4041 | UCAGAGACAUUAGUACGUG | 9531 | CACGUACUAAUGUCUCUGA |
| siRNA 4042 | 4042 | CAGAGACAUUAGUACGUGU | 9532 | ACACGUACUAAUGUCUCUG |
| siRNA 4043 | 4043 | AGAGACAUUAGUACGUGUC | 9533 | GACACGUACUAAUGUCUCU |
| siRNA 4044 | 4044 | GAGACAUUAGUACGUGUCC | 9534 | GGACACGUACUAAUGUCUC |
| siRNA 4045 | 4045 | AGACAUUAGUACGUGUCCA | 9535 | UGGACACGUACUAAUGUCU |
| siRNA 4046 | 4046 | GACAUUAGUACGUGUCCAA | 9536 | UUGGACACGUACUAAUGUC |
| siRNA 4047 | 4047 | ACAUUAGUACGUGUCCAAA | 9537 | UUUGGACACGUACUAAUGU |
| siRNA 4048 | 4048 | CAUUAGUACGUGUCCAAAU | 9538 | AUUUGGACACGUACUAAUG |
| siRNA 4049 | 4049 | AUUAGUACGUGUCCAAAUU | 9539 | AAUUUGGACACGUACUAAU |
| siRNA 4050 | 4050 | UUAGUACGUGUCCAAAUUC | 9540 | GAAUUUGGACACGUACUAA |
| siRNA 4051 | 4051 | UAGUACGUGUCCAAAUUCG | 9541 | CGAAUUUGGACACGUACUA |
| siRNA 4052 | 4052 | AGUACGUGUCCAAAUUCGC | 9542 | GCGAAUUUGGACACGUACU |
| siRNA 4053 | 4053 | GUACGUGUCCAAAUUCGCC | 9543 | GGCGAAUUUGGACACGUAC |
| siRNA 4054 | 4054 | UACGUGUCCAAAUUCGCCA | 9544 | UGGCGAAUUUGGACACGUA |
| siRNA 4055 | 4055 | ACGUGUCCAAAUUCGCCAA | 9545 | UUGGCGAAUUUGGACACGU |
| siRNA 4056 | 4056 | CGUGUCCAAAUUCGCCAAG | 9546 | CUUGGCGAAUUUGGACACG |
| siRNA 4057 | 4057 | GUGUCCAAAUUCGCCAAGC | 9547 | GCUUGGCGAAUUUGGACAC |
| siRNA 4058 | 4058 | UGUCCAAAUUCGCCAAGCA | 9548 | UGCUUGGCGAAUUUGGACA |
| siRNA 4059 | 4059 | GUCCAAAUUCGCCAAGCAC | 9549 | GUGCUUGGCGAAUUUGGAC |
| siRNA 4060 | 4060 | UCCAAAUUCGCCAAGCACU | 9550 | AGUGCUUGGCGAAUUUGGA |
| siRNA 4061 | 4061 | CCAAAUUCGCCAAGCACUC | 9551 | GAGUGCUUGGCGAAUUUGG |
| siRNA 4062 | 4062 | CAAAUUCGCCAAGCACUCC | 9552 | GGAGUGCUUGGCGAAUUUG |
| siRNA 4063 | 4063 | AAAUUCGCCAAGCACUCCU | 9553 | AGGAGUGCUUGGCGAAUUU |
| siRNA 4064 | 4064 | AAUUCGCCAAGCACUCCUC | 9554 | GAGGAGUGCUUGGCGAAUU |
| siRNA 4065 | 4065 | AUUCGCCAAGCACUCCUCC | 9555 | GGAGGAGUGCUUGGCGAAU |
| siRNA 4066 | 4066 | UUCGCCAAGCACUCCUCCU | 9556 | AGGAGGAGUGCUUGGCGAA |
| siRNA 4067 | 4067 | UCGCCAAGCACUCCUCCUA | 9557 | UAGGAGGAGUGCUUGGCGA |
| siRNA 4068 | 4068 | CGCCAAGCACUCCUCCUAG | 9558 | CUAGGAGGAGUGCUUGGCG |
| siRNA 4069 | 4069 | GCCAAGCACUCCUCCUAGC | 9559 | GCUAGGAGGAGUGCUUGGC |
| siRNA 4070 | 4070 | CCAAGCACUCCUCCUAGCA | 9560 | UGCUAGGAGGAGUGCUUGG |
| siRNA 4071 | 4071 | CAAGCACUCCUCCUAGCAC | 9561 | GUGCUAGGAGGAGUGCUUG |
| siRNA 4072 | 4072 | AAGCACUCCUCCUAGCACA | 9562 | UGUGCUAGGAGGAGUGCUU |
| siRNA 4073 | 4073 | AGCACUCCUCCUAGCACAC | 9563 | GUGUGCUAGGAGGAGUGCU |
| siRNA 4074 | 4074 | GCACUCCUCCUAGCACACC | 9564 | GGUGUGCUAGGAGGAGUGC |
| siRNA 4075 | 4075 | CACUCCUCCUAGCACACCC | 9565 | GGGUGUGCUAGGAGGAGUG |
| siRNA 4076 | 4076 | ACUCCUCCUAGCACACCCU | 9566 | AGGGUGUGCUAGGAGGAGU |
| siRNA 4077 | 4077 | CUCCUCCUAGCACACCCUC | 9567 | GAGGGUGUGCUAGGAGGAG |
| siRNA 4078 | 4078 | UCCUCCUAGCACACCCUCU | 9568 | AGAGGGUGUGCUAGGAGGA |
| siRNA 4079 | 4079 | CCUCCUAGCACACCCUCUC | 9569 | GAGAGGGUGUGCUAGGAGG |
| siRNA 4080 | 4080 | CUCCUAGCACACCCUCUCC | 9570 | GGAGAGGGUGUGCUAGGAG |
| siRNA 4081 | 4081 | UCCUAGCACACCCUCUCCA | 9571 | UGGAGAGGGUGUGCUAGGA |
| siRNA 4082 | 4082 | CCUAGCACACCCUCUCCAA | 9572 | UUGGAGAGGGUGUGCUAGG |
| siRNA 4083 | 4083 | CUAGCACACCCUCUCCAAG | 9573 | CUUGGAGAGGGUGUGCUAG |
| siRNA 4084 | 4084 | UAGCACACCCUCUCCAAGG | 9574 | CCUUGGAGAGGGUGUGCUA |
| siRNA 4085 | 4085 | AGCACACCCUCUCCAAGGG | 9575 | CCCUUGGAGAGGGUGUGCU |
| siRNA 4086 | 4086 | GCACACCCUCUCCAAGGGU | 9576 | ACCCUUGGAGAGGGUGUGC |
| siRNA 4087 | 4087 | CACACCCUCUCCAAGGGUA | 9577 | UACCCUUGGAGAGGGUGUG |
| siRNA 4088 | 4088 | ACACCCUCUCCAAGGGUAC | 9578 | GUACCCUUGGAGAGGGUGU |
| siRNA 4089 | 4089 | CACCCUCUCCAAGGGUACC | 9579 | GGUACCCUUGGAGAGGGUG |
| siRNA 4090 | 4090 | ACCCUCUCCAAGGGUACCG | 9580 | CGGUACCCUUGGAGAGGGU |
| siRNA 4091 | 4091 | CCCUCUCCAAGGGUACCGC | 9581 | GCGGUACCCUUGGAGAGGG |
| siRNA 4092 | 4092 | CCUCUCCAAGGGUACCGCG | 9582 | CGCGGUACCCUUGGAGAGG |
| siRNA 4093 | 4093 | CUCUCCAAGGGUACCGCGU | 9583 | ACGCGGUACCCUUGGAGAG |
| siRNA 4094 | 4094 | UCUCCAAGGGUACCGCGUC | 9584 | GACGCGGUACCCUUGGAGA |
| siRNA 4095 | 4095 | CUCCAAGGGUACCGCGUCG | 9585 | CGACGCGGUACCCUUGGAG |
| siRNA 4096 | 4096 | UCCAAGGGUACCGCGUCGA | 9586 | UCGACGCGGUACCCUUGGA |
| siRNA 4097 | 4097 | CCAAGGGUACCGCGUCGAU | 9587 | AUCGACGCGGUACCCUUGG |
| siRNA 4098 | 4098 | CAAGGGUACCGCGUCGAUG | 9588 | CAUCGACGCGGUACCCUUG |
| siRNA 4099 | 4099 | AAGGGUACCGCGUCGAUGC | 9589 | GCAUCGACGCGGUACCCUU |
| siRNA 4100 | 4100 | AGGGUACCGCGUCGAUGCU | 9590 | AGCAUCGACGCGGUACCCU |
| siRNA 4101 | 4101 | GGGUACCGCGUCGAUGCUA | 9591 | UAGCAUCGACGCGGUACCC |
| siRNA 4102 | 4102 | GGUACCGCGUCGAUGCUAU | 9592 | AUAGCAUCGACGCGGUACC |
| siRNA 4103 | 4103 | GUACCGCGUCGAUGCUAUG | 9593 | CAUAGCAUCGACGCGGUAC |
| siRNA 4104 | 4104 | UACCGCGUCGAUGCUAUGU | 9594 | ACAUAGCAUCGACGCGGUA |
| siRNA 4105 | 4105 | ACCGCGUCGAUGCUAUGUG | 9595 | CACAUAGCAUCGACGCGGU |
| siRNA 4106 | 4106 | CCGCGUCGAUGCUAUGUGC | 9596 | GCACAUAGCAUCGACGCGG |
| siRNA 4107 | 4107 | CGCGUCGAUGCUAUGUGCU | 9597 | AGCACAUAGCAUCGACGCG |
| siRNA 4108 | 4108 | GCGUCGAUGCUAUGUGCUC | 9598 | GAGCACAUAGCAUCGACGC |
| siRNA 4109 | 4109 | CGUCGAUGCUAUGUGCUCA | 9599 | UGAGCACAUAGCAUCGACG |
| siRNA 4110 | 4110 | GUCGAUGCUAUGUGCUCAG | 9600 | CUGAGCACAUAGCAUCGAC |
| siRNA 4111 | 4111 | UCGAUGCUAUGUGCUCAGU | 9601 | ACUGAGCACAUAGCAUCGA |
| siRNA 4112 | 4112 | CGAUGCUAUGUGCUCAGUU | 9602 | AACUGAGCACAUAGCAUCG |
| siRNA 4113 | 4113 | GAUGCUAUGUGCUCAGUUC | 9603 | GAACUGAGCACAUAGCAUC |
| siRNA 4114 | 4114 | AUGCUAUGUGCUCAGUUCU | 9604 | AGAACUGAGCACAUAGCAU |
| siRNA 4115 | 4115 | UGCUAUGUGCUCAGUUCUA | 9605 | UAGAACUGAGCACAUAGCA |
| siRNA 4116 | 4116 | GCUAUGUGCUCAGUUCUAG | 9606 | CUAGAACUGAGCACAUAGC |
| siRNA 4117 | 4117 | CUAUGUGCUCAGUUCUAGU | 9607 | ACUAGAACUGAGCACAUAG |
| siRNA 4118 | 4118 | UAUGUGCUCAGUUCUAGUC | 9608 | GACUAGAACUGAGCACAUA |
| siRNA 4119 | 4119 | AUGUGCUCAGUUCUAGUCA | 9609 | UGACUAGAACUGAGCACAU |
| siRNA 4120 | 4120 | UGUGCUCAGUUCUAGUCAG | 9610 | CUGACUAGAACUGAGCACA |
| siRNA 4121 | 4121 | GUGCUCAGUUCUAGUCAGA | 9611 | UCUGACUAGAACUGAGCAC |
| siRNA 4122 | 4122 | UGCUCAGUUCUAGUCAGAA | 9612 | UUCUGACUAGAACUGAGCA |
| siRNA 4123 | 4123 | GCUCAGUUCUAGUCAGAAU | 9613 | AUUCUGACUAGAACUGAGC |
| siRNA 4124 | 4124 | CUCAGUUCUAGUCAGAAUA | 9614 | UAUUCUGACUAGAACUGAG |
| siRNA 4125 | 4125 | UCAGUUCUAGUCAGAAUAA | 9615 | UUAUUCUGACUAGAACUGA |
| siRNA 4126 | 4126 | CAGUUCUAGUCAGAAUAAU | 9616 | AUUAUUCUGACUAGAACUG |
| siRNA 4127 | 4127 | AGUUCUAGUCAGAAUAAUC | 9617 | GAUUAUUCUGACUAGAACU |
| siRNA 4128 | 4128 | GUUCUAGUCAGAAUAAUCU | 9618 | AGAUUAUUCUGACUAGAAC |
| siRNA 4129 | 4129 | UUCUAGUCAGAAUAAUCUU | 9619 | AAGAUUAUUCUGACUAGAA |
| siRNA 4130 | 4130 | UCUAGUCAGAAUAAUCUUG | 9620 | CAAGAUUAUUCUGACUAGA |
| siRNA 4131 | 4131 | CUAGUCAGAAUAAUCUUGC | 9621 | GCAAGAUUAUUCUGACUAG |
| siRNA 4132 | 4132 | UAGUCAGAAUAAUCUUGCU | 9622 | AGCAAGAUUAUUCUGACUA |
| siRNA 4133 | 4133 | AGUCAGAAUAAUCUUGCUC | 9623 | GAGCAAGAUUAUUCUGACU |
| siRNA 4134 | 4134 | GUCAGAAUAAUCUUGCUCA | 9624 | UGAGCAAGAUUAUUCUGAC |
| siRNA 4135 | 4135 | UCAGAAUAAUCUUGCUCAU | 9625 | AUGAGCAAGAUUAUUCUGA |
| siRNA 4136 | 4136 | CAGAAUAAUCUUGCUCAUC | 9626 | GAUGAGCAAGAUUAUUCUG |
| siRNA 4137 | 4137 | AGAAUAAUCUUGCUCAUCC | 9627 | GGAUGAGCAAGAUUAUUCU |
| siRNA 4138 | 4138 | GAAUAAUCUUGCUCAUCCU | 9628 | AGGAUGAGCAAGAUUAUUC |
| siRNA 4139 | 4139 | AAUAAUCUUGCUCAUCCUC | 9629 | GAGGAUGAGCAAGAUUAUU |
| siRNA 4140 | 4140 | AUAAUCUUGCUCAUCCUCC | 9630 | GGAGGAUGAGCAAGAUUAU |
| siRNA 4141 | 4141 | UAAUCUUGCUCAUCCUCCA | 9631 | UGGAGGAUGAGCAAGAUUA |
| siRNA 4142 | 4142 | AAUCUUGCUCAUCCUCCAG | 9632 | CUGGAGGAUGAGCAAGAUU |
| siRNA 4143 | 4143 | AUCUUGCUCAUCCUCCAGC | 9633 | GCUGGAGGAUGAGCAAGAU |
| siRNA 4144 | 4144 | UCUUGCUCAUCCUCCAGCU | 9634 | AGCUGGAGGAUGAGCAAGA |
| siRNA 4145 | 4145 | CUUGCUCAUCCUCCAGCUC | 9635 | GAGCUGGAGGAUGAGCAAG |
| siRNA 4146 | 4146 | UUGCUCAUCCUCCAGCUCC | 9636 | GGAGCUGGAGGAUGAGCAA |
| siRNA 4147 | 4147 | UGCUCAUCCUCCAGCUCCC | 9637 | GGGAGCUGGAGGAUGAGCA |
| siRNA 4148 | 4148 | GCUCAUCCUCCAGCUCCCC | 9638 | GGGGAGCUGGAGGAUGAGC |
| siRNA 4149 | 4149 | CUCAUCCUCCAGCUCCCCC | 9639 | GGGGGAGCUGGAGGAUGAG |
| siRNA 4150 | 4150 | UCAUCCUCCAGCUCCCCCU | 9640 | AGGGGGAGCUGGAGGAUGA |
| siRNA 4151 | 4151 | CAUCCUCCAGCUCCCCCUG | 9641 | CAGGGGGAGCUGGAGGAUG |
| siRNA 4152 | 4152 | AUCCUCCAGCUCCCCCUGU | 9642 | ACAGGGGGAGCUGGAGGAU |
| siRNA 4153 | 4153 | UCCUCCAGCUCCCCCUGUU | 9643 | AACAGGGGGAGCUGGAGGA |
| siRNA 4154 | 4154 | CCUCCAGCUCCCCCUGUUC | 9644 | GAACAGGGGGAGCUGGAGG |
| siRNA 4155 | 4155 | CUCCAGCUCCCCCUGUUCC | 9645 | GGAACAGGGGGAGCUGGAG |
| siRNA 4156 | 4156 | UCCAGCUCCCCCUGUUCCA | 9646 | UGGAACAGGGGGAGCUGGA |
| siRNA 4157 | 4157 | CCAGCUCCCCCUGUUCCAC | 9647 | GUGGAACAGGGGGAGCUGG |
| siRNA 4158 | 4158 | CAGCUCCCCCUGUUCCACC | 9648 | GGUGGAACAGGGGGAGCUG |
| siRNA 4159 | 4159 | AGCUCCCCCUGUUCCACCA | 9649 | UGGUGGAACAGGGGGAGCU |
| siRNA 4160 | 4160 | GCUCCCCCUGUUCCACCAA | 9650 | UUGGUGGAACAGGGGGAGC |
| siRNA 4161 | 4161 | CUCCCCCUGUUCCACCAAG | 9651 | CUUGGUGGAACAGGGGGAG |
| siRNA 4162 | 4162 | UCCCCCUGUUCCACCAAGG | 9652 | CCUUGGUGGAACAGGGGGA |
| siRNA 4163 | 4163 | CCCCCUGUUCCACCAAGGC | 9653 | GCCUUGGUGGAACAGGGGG |
| siRNA 4164 | 4164 | CCCCUGUUCCACCAAGGCA | 9654 | UGCCUUGGUGGAACAGGGG |
| siRNA 4165 | 4165 | CCCUGUUCCACCAAGGCAG | 9655 | CUGCCUUGGUGGAACAGGG |
| siRNA 4166 | 4166 | CCUGUUCCACCAAGGCAGA | 9656 | UCUGCCUUGGUGGAACAGG |
| siRNA 4167 | 4167 | CUGUUCCACCAAGGCAGAA | 9657 | UUCUGCCUUGGUGGAACAG |
| siRNA 4168 | 4168 | UGUUCCACCAAGGCAGAAU | 9658 | AUUCUGCCUUGGUGGAACA |
| siRNA 4169 | 4169 | GUUCCACCAAGGCAGAAUU | 9659 | AAUUCUGCCUUGGUGGAAC |
| siRNA 4170 | 4170 | UUCCACCAAGGCAGAAUUC | 9660 | GAAUUCUGCCUUGGUGGAA |
| siRNA 4171 | 4171 | UCCACCAAGGCAGAAUUCA | 9661 | UGAAUUCUGCCUUGGUGGA |
| siRNA 4172 | 4172 | CCACCAAGGCAGAAUUCAA | 9662 | UUGAAUUCUGCCUUGGUGG |
| siRNA 4173 | 4173 | CACCAAGGCAGAAUUCAAG | 9663 | CUUGAAUUCUGCCUUGGUG |
| siRNA 4174 | 4174 | ACCAAGGCAGAAUUCAAGC | 9664 | GCUUGAAUUCUGCCUUGGU |
| siRNA 4175 | 4175 | CCAAGGCAGAAUUCAAGCC | 9665 | GGCUUGAAUUCUGCCUUGG |
| siRNA 4176 | 4176 | CAAGGCAGAAUUCAAGCCC | 9666 | GGGCUUGAAUUCUGCCUUG |
| siRNA 4177 | 4177 | AAGGCAGAAUUCAAGCCCU | 9667 | AGGGCUUGAAUUCUGCCUU |
| siRNA 4178 | 4178 | AGGCAGAAUUCAAGCCCUC | 9668 | GAGGGCUUGAAUUCUGCCU |
| siRNA 4179 | 4179 | GGCAGAAUUCAAGCCCUCA | 9669 | UGAGGGCUUGAAUUCUGCC |
| siRNA 4180 | 4180 | GCAGAAUUCAAGCCCUCAU | 9670 | AUGAGGGCUUGAAUUCUGC |
| siRNA 4181 | 4181 | CAGAAUUCAAGCCCUCAUC | 9671 | GAUGAGGGCUUGAAUUCUG |
| siRNA 4182 | 4182 | AGAAUUCAAGCCCUCAUCU | 9672 | AGAUGAGGGCUUGAAUUCU |
| siRNA 4183 | 4183 | GAAUUCAAGCCCUCAUCUG | 9673 | CAGAUGAGGGCUUGAAUUC |
| siRNA 4184 | 4184 | AAUUCAAGCCCUCAUCUGC | 9674 | GCAGAUGAGGGCUUGAAUU |
| siRNA 4185 | 4185 | AUUCAAGCCCUCAUCUGCC | 9675 | GGCAGAUGAGGGCUUGAAU |
| siRNA 4186 | 4186 | UUCAAGCCCUCAUCUGCCA | 9676 | UGGCAGAUGAGGGCUUGAA |
| siRNA 4187 | 4187 | UCAAGCCCUCAUCUGCCAA | 9677 | UUGGCAGAUGAGGGCUUGA |
| siRNA 4188 | 4188 | CAAGCCCUCAUCUGCCAAA | 9678 | UUUGGCAGAUGAGGGCUUG |
| siRNA 4189 | 4189 | AAGCCCUCAUCUGCCAAAA | 9679 | UUUUGGCAGAUGAGGGCUU |
| siRNA 4190 | 4190 | AGCCCUCAUCUGCCAAAAC | 9680 | GUUUUGGCAGAUGAGGGCU |
| siRNA 4191 | 4191 | GCCCUCAUCUGCCAAAACU | 9681 | AGUUUUGGCAGAUGAGGGC |
| siRNA 4192 | 4192 | CCCUCAUCUGCCAAAACUG | 9682 | CAGUUUUGGCAGAUGAGGG |
| siRNA 4193 | 4193 | CCUCAUCUGCCAAAACUGC | 9683 | GCAGUUUUGGCAGAUGAGG |
| siRNA 4194 | 4194 | CUCAUCUGCCAAAACUGCC | 9684 | GGCAGUUUUGGCAGAUGAG |
| siRNA 4195 | 4195 | UCAUCUGCCAAAACUGCCA | 9685 | UGGCAGUUUUGGCAGAUGA |
| siRNA 4196 | 4196 | CAUCUGCCAAAACUGCCAC | 9686 | GUGGCAGUUUUGGCAGAUG |
| siRNA 4197 | 4197 | AUCUGCCAAAACUGCCACC | 9687 | GGUGGCAGUUUUGGCAGAU |
| siRNA 4198 | 4198 | UCUGCCAAAACUGCCACCA | 9688 | UGGUGGCAGUUUUGGCAGA |
| siRNA 4199 | 4199 | CUGCCAAAACUGCCACCAA | 9689 | UUGGUGGCAGUUUUGGCAG |
| siRNA 4200 | 4200 | UGCCAAAACUGCCACCAAA | 9690 | UUUGGUGGCAGUUUUGGCA |
| siRNA 4201 | 4201 | GCCAAAACUGCCACCAAAG | 9691 | CUUUGGUGGCAGUUUUGGC |
| siRNA 4202 | 4202 | CCAAAACUGCCACCAAAGA | 9692 | UCUUUGGUGGCAGUUUUGG |
| siRNA 4203 | 4203 | CAAAACUGCCACCAAAGAC | 9693 | GUCUUUGGUGGCAGUUUUG |
| siRNA 4204 | 4204 | AAAACUGCCACCAAAGACU | 9694 | AGUCUUUGGUGGCAGUUUU |
| siRNA 4205 | 4205 | AAACUGCCACCAAAGACUU | 9695 | AAGUCUUUGGUGGCAGUUU |
| siRNA 4206 | 4206 | AACUGCCACCAAAGACUUA | 9696 | UAAGUCUUUGGUGGCAGUU |
| siRNA 4207 | 4207 | ACUGCCACCAAAGACUUAC | 9697 | GUAAGUCUUUGGUGGCAGU |
| siRNA 4208 | 4208 | CUGCCACCAAAGACUUACA | 9698 | UGUAAGUCUUUGGUGGCAG |
| siRNA 4209 | 4209 | UGCCACCAAAGACUUACAA | 9699 | UUGUAAGUCUUUGGUGGCA |
| siRNA 4210 | 4210 | GCCACCAAAGACUUACAAA | 9700 | UUUGUAAGUCUUUGGUGGC |
| siRNA 4211 | 4211 | CCACCAAAGACUUACAAAC | 9701 | GUUUGUAAGUCUUUGGUGG |
| siRNA 4212 | 4212 | CACCAAAGACUUACAAACG | 9702 | CGUUUGUAAGUCUUUGGUG |
| siRNA 4213 | 4213 | ACCAAAGACUUACAAACGG | 9703 | CCGUUUGUAAGUCUUUGGU |
| siRNA 4214 | 4214 | CCAAAGACUUACAAACGGG | 9704 | CCCGUUUGUAAGUCUUUGG |
| siRNA 4215 | 4215 | CAAAGACUUACAAACGGGA | 9705 | UCCCGUUUGUAAGUCUUUG |
| siRNA 4216 | 4216 | AAAGACUUACAAACGGGAG | 9706 | CUCCCGUUUGUAAGUCUUU |
| siRNA 4217 | 4217 | AAGACUUACAAACGGGAGC | 9707 | GCUCCCGUUUGUAAGUCUU |
| siRNA 4218 | 4218 | AGACUUACAAACGGGAGCU | 9708 | AGCUCCCGUUUGUAAGUCU |
| siRNA 4219 | 4219 | GACUUACAAACGGGAGCUU | 9709 | AAGCUCCCGUUUGUAAGUC |
| siRNA 4220 | 4220 | ACUUACAAACGGGAGCUUU | 9710 | AAAGCUCCCGUUUGUAAGU |
| siRNA 4221 | 4221 | CUUACAAACGGGAGCUUUC | 9711 | GAAAGCUCCCGUUUGUAAG |
| siRNA 4222 | 4222 | UUACAAACGGGAGCUUUCG | 9712 | CGAAAGCUCCCGUUUGUAA |
| siRNA 4223 | 4223 | UACAAACGGGAGCUUUCGC | 9713 | GCGAAAGCUCCCGUUUGUA |
| siRNA 4224 | 4224 | ACAAACGGGAGCUUUCGCA | 9714 | UGCGAAAGCUCCCGUUUGU |
| siRNA 4225 | 4225 | CAAACGGGAGCUUUCGCAC | 9715 | GUGCGAAAGCUCCCGUUUG |
| siRNA 4226 | 4226 | AAACGGGAGCUUUCGCACC | 9716 | GGUGCGAAAGCUCCCGUUU |
| siRNA 4227 | 4227 | AACGGGAGCUUUCGCACCC | 9717 | GGGUGCGAAAGCUCCCGUU |
| siRNA 4228 | 4228 | ACGGGAGCUUUCGCACCCC | 9718 | GGGGUGCGAAAGCUCCCGU |
| siRNA 4229 | 4229 | CGGGAGCUUUCGCACCCCC | 9719 | GGGGGUGCGAAAGCUCCCG |
| siRNA 4230 | 4230 | GGGAGCUUUCGCACCCCCC | 9720 | GGGGGGUGCGAAAGCUCCC |
| siRNA 4231 | 4231 | GGAGCUUUCGCACCCCCCA | 9721 | UGGGGGGUGCGAAAGCUCC |
| siRNA 4232 | 4232 | GAGCUUUCGCACCCCCCAU | 9722 | AUGGGGGGUGCGAAAGCUC |
| siRNA 4233 | 4233 | AGCUUUCGCACCCCCCAUU | 9723 | AAUGGGGGGUGCGAAAGCU |
| siRNA 4234 | 4234 | GCUUUCGCACCCCCCAUUG | 9724 | CAAUGGGGGGUGCGAAAGC |
| siRNA 4235 | 4235 | CUUUCGCACCCCCCAUUGU | 9725 | ACAAUGGGGGGUGCGAAAG |
| siRNA 4236 | 4236 | UUUCGCACCCCCCAUUGUA | 9726 | UACAAUGGGGGGUGCGAAA |
| siRNA 4237 | 4237 | UUCGCACCCCCCAUUGUAC | 9727 | GUACAAUGGGGGGUGCGAA |
| siRNA 4238 | 4238 | UCGCACCCCCCAUUGUACA | 9728 | UGUACAAUGGGGGGUGCGA |
| siRNA 4239 | 4239 | CGCACCCCCCAUUGUACAG | 9729 | CUGUACAAUGGGGGGUGCG |
| siRNA 4240 | 4240 | GCACCCCCCAUUGUACAGA | 9730 | UCUGUACAAUGGGGGGUGC |
| siRNA 4241 | 4241 | CACCCCCCAUUGUACAGAC | 9731 | GUCUGUACAAUGGGGGGUG |
| siRNA 4242 | 4242 | ACCCCCCAUUGUACAGACU | 9732 | AGUCUGUACAAUGGGGGGU |
| siRNA 4243 | 4243 | CCCCCCAUUGUACAGACUG | 9733 | CAGUCUGUACAAUGGGGGG |
| siRNA 4244 | 4244 | CCCCCAUUGUACAGACUGC | 9734 | GCAGUCUGUACAAUGGGGG |
| siRNA 4245 | 4245 | CCCCAUUGUACAGACUGCC | 9735 | GGCAGUCUGUACAAUGGGG |
| siRNA 4246 | 4246 | CCCAUUGUACAGACUGCCU | 9736 | AGGCAGUCUGUACAAUGGG |
| siRNA 4247 | 4247 | CCAUUGUACAGACUGCCUU | 9737 | AAGGCAGUCUGUACAAUGG |
| siRNA 4248 | 4248 | CAUUGUACAGACUGCCUUU | 9738 | AAAGGCAGUCUGUACAAUG |
| siRNA 4249 | 4249 | AUUGUACAGACUGCCUUUG | 9739 | CAAAGGCAGUCUGUACAAU |
| siRNA 4250 | 4250 | UUGUACAGACUGCCUUUGC | 9740 | GCAAAGGCAGUCUGUACAA |
| siRNA 4251 | 4251 | UGUACAGACUGCCUUUGCU | 9741 | AGCAAAGGCAGUCUGUACA |
| siRNA 4252 | 4252 | GUACAGACUGCCUUUGCUA | 9742 | UAGCAAAGGCAGUCUGUAC |
| siRNA 4253 | 4253 | UACAGACUGCCUUUGCUAG | 9743 | CUAGCAAAGGCAGUCUGUA |
| siRNA 4254 | 4254 | ACAGACUGCCUUUGCUAGA | 9744 | UCUAGCAAAGGCAGUCUGU |
| siRNA 4255 | 4255 | CAGACUGCCUUUGCUAGAA | 9745 | UUCUAGCAAAGGCAGUCUG |
| siRNA 4256 | 4256 | AGACUGCCUUUGCUAGAAA | 9746 | UUUCUAGCAAAGGCAGUCU |
| siRNA 4257 | 4257 | GACUGCCUUUGCUAGAAAA | 9747 | UUUUCUAGCAAAGGCAGUC |
| siRNA 4258 | 4258 | ACUGCCUUUGCUAGAAAAU | 9748 | AUUUUCUAGCAAAGGCAGU |
| siRNA 4259 | 4259 | CUGCCUUUGCUAGAAAAUG | 9749 | CAUUUUCUAGCAAAGGCAG |
| siRNA 4260 | 4260 | UGCCUUUGCUAGAAAAUGC | 9750 | GCAUUUUCUAGCAAAGGCA |
| siRNA 4261 | 4261 | GCCUUUGCUAGAAAAUGCA | 9751 | UGCAUUUUCUAGCAAAGGC |
| siRNA 4262 | 4262 | CCUUUGCUAGAAAAUGCAG | 9752 | CUGCAUUUUCUAGCAAAGG |
| siRNA 4263 | 4263 | CUUUGCUAGAAAAUGCAGA | 9753 | UCUGCAUUUUCUAGCAAAG |
| siRNA 4264 | 4264 | UUUGCUAGAAAAUGCAGAA | 9754 | UUCUGCAUUUUCUAGCAAA |
| siRNA 4265 | 4265 | UUGCUAGAAAAUGCAGAAA | 9755 | UUUCUGCAUUUUCUAGCAA |
| siRNA 4266 | 4266 | UGCUAGAAAAUGCAGAAAC | 9756 | GUUUCUGCAUUUUCUAGCA |
| siRNA 4267 | 4267 | GCUAGAAAAUGCAGAAACU | 9757 | AGUUUCUGCAUUUUCUAGC |
| siRNA 4268 | 4268 | CUAGAAAAUGCAGAAACUC | 9758 | GAGUUUCUGCAUUUUCUAG |
| siRNA 4269 | 4269 | UAGAAAAUGCAGAAACUCC | 9759 | GGAGUUUCUGCAUUUUCUA |
| siRNA 4270 | 4270 | AGAAAAUGCAGAAACUCCC | 9760 | GGGAGUUUCUGCAUUUUCU |
| siRNA 4271 | 4271 | GAAAAUGCAGAAACUCCCC | 9761 | GGGGAGUUUCUGCAUUUUC |
| siRNA 4272 | 4272 | AAAAUGCAGAAACUCCCCA | 9762 | UGGGGAGUUUCUGCAUUUU |
| siRNA 4273 | 4273 | AAAUGCAGAAACUCCCCAA | 9763 | UUGGGGAGUUUCUGCAUUU |
| siRNA 4274 | 4274 | AAUGCAGAAACUCCCCAAU | 9764 | AUUGGGGAGUUUCUGCAUU |
| siRNA 4275 | 4275 | AUGCAGAAACUCCCCAAUG | 9765 | CAUUGGGGAGUUUCUGCAU |
| siRNA 4276 | 4276 | UGCAGAAACUCCCCAAUGA | 9766 | UCAUUGGGGAGUUUCUGCA |
| siRNA 4277 | 4277 | GCAGAAACUCCCCAAUGAC | 9767 | GUCAUUGGGGAGUUUCUGC |
| siRNA 4278 | 4278 | CAGAAACUCCCCAAUGACC | 9768 | GGUCAUUGGGGAGUUUCUG |
| siRNA 4279 | 4279 | AGAAACUCCCCAAUGACCU | 9769 | AGGUCAUUGGGGAGUUUCU |
| siRNA 4280 | 4280 | GAAACUCCCCAAUGACCUU | 9770 | AAGGUCAUUGGGGAGUUUC |
| siRNA 4281 | 4281 | AAACUCCCCAAUGACCUUA | 9771 | UAAGGUCAUUGGGGAGUUU |
| siRNA 4282 | 4282 | AACUCCCCAAUGACCUUAG | 9772 | CUAAGGUCAUUGGGGAGUU |
| siRNA 4283 | 4283 | ACUCCCCAAUGACCUUAGC | 9773 | GCUAAGGUCAUUGGGGAGU |
| siRNA 4284 | 4284 | CUCCCCAAUGACCUUAGCC | 9774 | GGCUAAGGUCAUUGGGGAG |
| siRNA 4285 | 4285 | UCCCCAAUGACCUUAGCCA | 9775 | UGGCUAAGGUCAUUGGGGA |
| siRNA 4286 | 4286 | CCCCAAUGACCUUAGCCAU | 9776 | AUGGCUAAGGUCAUUGGGG |
| siRNA 4287 | 4287 | CCCAAUGACCUUAGCCAUA | 9777 | UAUGGCUAAGGUCAUUGGG |
| siRNA 4288 | 4288 | CCAAUGACCUUAGCCAUAU | 9778 | AUAUGGCUAAGGUCAUUGG |
| siRNA 4289 | 4289 | CAAUGACCUUAGCCAUAUG | 9779 | CAUAUGGCUAAGGUCAUUG |
| siRNA 4290 | 4290 | AAUGACCUUAGCCAUAUGU | 9780 | ACAUAUGGCUAAGGUCAUU |
| siRNA 4291 | 4291 | AUGACCUUAGCCAUAUGUA | 9781 | UACAUAUGGCUAAGGUCAU |
| siRNA 4292 | 4292 | UGACCUUAGCCAUAUGUAG | 9782 | CUACAUAUGGCUAAGGUCA |
| siRNA 4293 | 4293 | GACCUUAGCCAUAUGUAGU | 9783 | ACUACAUAUGGCUAAGGUC |
| siRNA 4294 | 4294 | ACCUUAGCCAUAUGUAGUC | 9784 | GACUACAUAUGGCUAAGGU |
| siRNA 4295 | 4295 | CCUUAGCCAUAUGUAGUCA | 9785 | UGACUACAUAUGGCUAAGG |
| siRNA 4296 | 4296 | CUUAGCCAUAUGUAGUCAU | 9786 | AUGACUACAUAUGGCUAAG |
| siRNA 4297 | 4297 | UUAGCCAUAUGUAGUCAUU | 9787 | AAUGACUACAUAUGGCUAA |
| siRNA 4298 | 4298 | UAGCCAUAUGUAGUCAUUG | 9788 | CAAUGACUACAUAUGGCUA |
| siRNA 4299 | 4299 | AGCCAUAUGUAGUCAUUGA | 9789 | UCAAUGACUACAUAUGGCU |
| siRNA 4300 | 4300 | GCCAUAUGUAGUCAUUGAC | 9790 | GUCAAUGACUACAUAUGGC |
| siRNA 4301 | 4301 | CCAUAUGUAGUCAUUGACA | 9791 | UGUCAAUGACUACAUAUGG |
| siRNA 4302 | 4302 | CAUAUGUAGUCAUUGACAC | 9792 | GUGUCAAUGACUACAUAUG |
| siRNA 4303 | 4303 | AUAUGUAGUCAUUGACACU | 9793 | AGUGUCAAUGACUACAUAU |
| siRNA 4304 | 4304 | UAUGUAGUCAUUGACACUG | 9794 | CAGUGUCAAUGACUACAUA |
| siRNA 4305 | 4305 | AUGUAGUCAUUGACACUGG | 9795 | CCAGUGUCAAUGACUACAU |
| siRNA 4306 | 4306 | UGUAGUCAUUGACACUGGA | 9796 | UCCAGUGUCAAUGACUACA |
| siRNA 4307 | 4307 | GUAGUCAUUGACACUGGAA | 9797 | UUCCAGUGUCAAUGACUAC |
| siRNA 4308 | 4308 | UAGUCAUUGACACUGGAAU | 9798 | AUUCCAGUGUCAAUGACUA |
| siRNA 4309 | 4309 | AGUCAUUGACACUGGAAUG | 9799 | CAUUCCAGUGUCAAUGACU |
| siRNA 4310 | 4310 | GUCAUUGACACUGGAAUGG | 9800 | CCAUUCCAGUGUCAAUGAC |
| siRNA 4311 | 4311 | UCAUUGACACUGGAAUGGU | 9801 | ACCAUUCCAGUGUCAAUGA |
| siRNA 4312 | 4312 | CAUUGACACUGGAAUGGUA | 9802 | UACCAUUCCAGUGUCAAUG |
| siRNA 4313 | 4313 | AUUGACACUGGAAUGGUAU | 9803 | AUACCAUUCCAGUGUCAAU |
| siRNA 4314 | 4314 | UUGACACUGGAAUGGUAUU | 9804 | AAUACCAUUCCAGUGUCAA |
| siRNA 4315 | 4315 | UGACACUGGAAUGGUAUUU | 9805 | AAAUACCAUUCCAGUGUCA |
| siRNA 4316 | 4316 | GACACUGGAAUGGUAUUUG | 9806 | CAAAUACCAUUCCAGUGUC |
| siRNA 4317 | 4317 | ACACUGGAAUGGUAUUUGU | 9807 | ACAAAUACCAUUCCAGUGU |
| siRNA 4318 | 4318 | CACUGGAAUGGUAUUUGUA | 9808 | UACAAAUACCAUUCCAGUG |
| siRNA 4319 | 4319 | ACUGGAAUGGUAUUUGUAA | 9809 | UUACAAAUACCAUUCCAGU |
| siRNA 4320 | 4320 | CUGGAAUGGUAUUUGUAAA | 9810 | UUUACAAAUACCAUUCCAG |
| siRNA 4321 | 4321 | UGGAAUGGUAUUUGUAAAG | 9811 | CUUUACAAAUACCAUUCCA |
| siRNA 4322 | 4322 | GGAAUGGUAUUUGUAAAGU | 9812 | ACUUUACAAAUACCAUUCC |
| siRNA 4323 | 4323 | GAAUGGUAUUUGUAAAGUU | 9813 | AACUUUACAAAUACCAUUC |
| siRNA 4324 | 4324 | AAUGGUAUUUGUAAAGUUU | 9814 | AAACUUUACAAAUACCAUU |
| siRNA 4325 | 4325 | AUGGUAUUUGUAAAGUUUU | 9815 | AAAACUUUACAAAUACCAU |
| siRNA 4326 | 4326 | UGGUAUUUGUAAAGUUUUU | 9816 | AAAAACUUUACAAAUACCA |
| siRNA 4327 | 4327 | GGUAUUUGUAAAGUUUUUU | 9817 | AAAAAACUUUACAAAUACC |
| siRNA 4328 | 4328 | GUAUUUGUAAAGUUUUUUU | 9818 | AAAAAAACUUUACAAAUAC |
| siRNA 4329 | 4329 | UAUUUGUAAAGUUUUUUUU | 9819 | AAAAAAAACUUUACAAAUA |
| siRNA 4330 | 4330 | AUUUGUAAAGUUUUUUUUU | 9820 | AAAAAAAAACUUUACAAAU |
| siRNA 4331 | 4331 | UUUGUAAAGUUUUUUUUUU | 9821 | AAAAAAAAAACUUUACAAA |
| siRNA 4332 | 4332 | UUGUAAAGUUUUUUUUUUA | 9822 | UAAAAAAAAAACUUUACAA |
| siRNA 4333 | 4333 | UGUAAAGUUUUUUUUUUAA | 9823 | UUAAAAAAAAAACUUUACA |
| siRNA 4334 | 4334 | GUAAAGUUUUUUUUUUAAU | 9824 | AUUAAAAAAAAAACUUUAC |
| siRNA 4335 | 4335 | UAAAGUUUUUUUUUUAAUU | 9825 | AAUUAAAAAAAAAACUUUA |
| siRNA 4336 | 4336 | AAAGUUUUUUUUUUAAUUU | 9826 | AAAUUAAAAAAAAAACUUU |
| siRNA 4337 | 4337 | AAGUUUUUUUUUUAAUUUA | 9827 | UAAAUUAAAAAAAAAACUU |
| siRNA 4338 | 4338 | AGUUUUUUUUUUAAUUUAU | 9828 | AUAAAUUAAAAAAAAAACU |
| siRNA 4339 | 4339 | GUUUUUUUUUUAAUUUAUU | 9829 | AAUAAAUUAAAAAAAAAAC |
| siRNA 4340 | 4340 | UUUUUUUUUUAAUUUAUUC | 9830 | GAAUAAAUUAAAAAAAAAA |
| siRNA 4341 | 4341 | UUUUUUUUUAAUUUAUUCA | 9831 | UGAAUAAAUUAAAAAAAAA |
| siRNA 4342 | 4342 | UUUUUUUUAAUUUAUUCAA | 9832 | UUGAAUAAAUUAAAAAAAA |
| siRNA 4343 | 4343 | UUUUUUUAAUUUAUUCAAA | 9833 | UUUGAAUAAAUUAAAAAAA |
| siRNA 4344 | 4344 | UUUUUUAAUUUAUUCAAAA | 9834 | UUUUGAAUAAAUUAAAAAA |
| siRNA 4345 | 4345 | UUUUUAAUUUAUUCAAAAA | 9835 | UUUUUGAAUAAAUUAAAAA |
| siRNA 4346 | 4346 | UUUUAAUUUAUUCAAAAAA | 9836 | UUUUUUGAAUAAAUUAAAA |
| siRNA 4347 | 4347 | UUUAAUUUAUUCAAAAAAA | 9837 | UUUUUUUGAAUAAAUUAAA |
| siRNA 4348 | 4348 | UUAAUUUAUUCAAAAAAAG | 9838 | CUUUUUUUGAAUAAAUUAA |
| siRNA 4349 | 4349 | UAAUUUAUUCAAAAAAAGA | 9839 | UCUUUUUUUGAAUAAAUUA |
| siRNA 4350 | 4350 | AAUUUAUUCAAAAAAAGAC | 9840 | GUCUUUUUUUGAAUAAAUU |
| siRNA 4351 | 4351 | AUUUAUUCAAAAAAAGACA | 9841 | UGUCUUUUUUUGAAUAAAU |
| siRNA 4352 | 4352 | UUUAUUCAAAAAAAGACAU | 9842 | AUGUCUUUUUUUGAAUAAA |
| siRNA 4353 | 4353 | UUAUUCAAAAAAAGACAUA | 9843 | UAUGUCUUUUUUUGAAUAA |
| siRNA 4354 | 4354 | UAUUCAAAAAAAGACAUAG | 9844 | CUAUGUCUUUUUUUGAAUA |
| siRNA 4355 | 4355 | AUUCAAAAAAAGACAUAGU | 9845 | ACUAUGUCUUUUUUUGAAU |
| siRNA 4356 | 4356 | UUCAAAAAAAGACAUAGUA | 9846 | UACUAUGUCUUUUUUUGAA |
| siRNA 4357 | 4357 | UCAAAAAAAGACAUAGUAU | 9847 | AUACUAUGUCUUUUUUUGA |
| siRNA 4358 | 4358 | CAAAAAAAGACAUAGUAUU | 9848 | AAUACUAUGUCUUUUUUUG |
| siRNA 4359 | 4359 | AAAAAAAGACAUAGUAUUU | 9849 | AAAUACUAUGUCUUUUUUU |
| siRNA 4360 | 4360 | AAAAAAGACAUAGUAUUUU | 9850 | AAAAUACUAUGUCUUUUUU |
| siRNA 4361 | 4361 | AAAAAGACAUAGUAUUUUA | 9851 | UAAAAUACUAUGUCUUUUU |
| siRNA 4362 | 4362 | AAAAGACAUAGUAUUUUAG | 9852 | CUAAAAUACUAUGUCUUUU |
| siRNA 4363 | 4363 | AAAGACAUAGUAUUUUAGU | 9853 | ACUAAAAUACUAUGUCUUU |
| siRNA 4364 | 4364 | AAGACAUAGUAUUUUAGUA | 9854 | UACUAAAAUACUAUGUCUU |
| siRNA 4365 | 4365 | AGACAUAGUAUUUUAGUAC | 9855 | GUACUAAAAUACUAUGUCU |
| siRNA 4366 | 4366 | GACAUAGUAUUUUAGUACU | 9856 | AGUACUAAAAUACUAUGUC |
| siRNA 4367 | 4367 | ACAUAGUAUUUUAGUACUU | 9857 | AAGUACUAAAAUACUAUGU |
| siRNA 4368 | 4368 | CAUAGUAUUUUAGUACUUU | 9858 | AAAGUACUAAAAUACUAUG |
| siRNA 4369 | 4369 | AUAGUAUUUUAGUACUUUU | 9859 | AAAAGUACUAAAAUACUAU |
| siRNA 4370 | 4370 | UAGUAUUUUAGUACUUUUU | 9860 | AAAAAGUACUAAAAUACUA |
| siRNA 4371 | 4371 | AGUAUUUUAGUACUUUUUA | 9861 | UAAAAAGUACUAAAAUACU |
| siRNA 4372 | 4372 | GUAUUUUAGUACUUUUUAC | 9862 | GUAAAAAGUACUAAAAUAC |
| siRNA 4373 | 4373 | UAUUUUAGUACUUUUUACA | 9863 | UGUAAAAAGUACUAAAAUA |
| siRNA 4374 | 4374 | AUUUUAGUACUUUUUACAA | 9864 | UUGUAAAAAGUACUAAAAU |
| siRNA 4375 | 4375 | UUUUAGUACUUUUUACAAA | 9865 | UUUGUAAAAAGUACUAAAA |
| siRNA 4376 | 4376 | UUUAGUACUUUUUACAAAU | 9866 | AUUUGUAAAAAGUACUAAA |
| siRNA 4377 | 4377 | UUAGUACUUUUUACAAAUA | 9867 | UAUUUGUAAAAAGUACUAA |
| siRNA 4378 | 4378 | UAGUACUUUUUACAAAUAA | 9868 | UUAUUUGUAAAAAGUACUA |
| siRNA 4379 | 4379 | AGUACUUUUUACAAAUAAU | 9869 | AUUAUUUGUAAAAAGUACU |
| siRNA 4380 | 4380 | GUACUUUUUACAAAUAAUG | 9870 | CAUUAUUUGUAAAAAGUAC |
| siRNA 4381 | 4381 | UACUUUUUACAAAUAAUGC | 9871 | GCAUUAUUUGUAAAAAGUA |
| siRNA 4382 | 4382 | ACUUUUUACAAAUAAUGCU | 9872 | AGCAUUAUUUGUAAAAAGU |
| siRNA 4383 | 4383 | CUUUUUACAAAUAAUGCUC | 9873 | GAGCAUUAUUUGUAAAAAG |
| siRNA 4384 | 4384 | UUUUUACAAAUAAUGCUCU | 9874 | AGAGCAUUAUUUGUAAAAA |
| siRNA 4385 | 4385 | UUUUACAAAUAAUGCUCUU | 9875 | AAGAGCAUUAUUUGUAAAA |
| siRNA 4386 | 4386 | UUUACAAAUAAUGCUCUUA | 9876 | UAAGAGCAUUAUUUGUAAA |
| siRNA 4387 | 4387 | UUACAAAUAAUGCUCUUAC | 9877 | GUAAGAGCAUUAUUUGUAA |
| siRNA 4388 | 4388 | UACAAAUAAUGCUCUUACA | 9878 | UGUAAGAGCAUUAUUUGUA |
| siRNA 4389 | 4389 | ACAAAUAAUGCUCUUACAA | 9879 | UUGUAAGAGCAUUAUUUGU |
| siRNA 4390 | 4390 | CAAAUAAUGCUCUUACAAA | 9880 | UUUGUAAGAGCAUUAUUUG |
| siRNA 4391 | 4391 | AAAUAAUGCUCUUACAAAA | 9881 | UUUUGUAAGAGCAUUAUUU |
| siRNA 4392 | 4392 | AAUAAUGCUCUUACAAAAA | 9882 | UUUUUGUAAGAGCAUUAUU |
| siRNA 4393 | 4393 | AUAAUGCUCUUACAAAAAU | 9883 | AUUUUUGUAAGAGCAUUAU |
| siRNA 4394 | 4394 | UAAUGCUCUUACAAAAAUG | 9884 | CAUUUUUGUAAGAGCAUUA |
| siRNA 4395 | 4395 | AAUGCUCUUACAAAAAUGC | 9885 | GCAUUUUUGUAAGAGCAUU |
| siRNA 4396 | 4396 | AUGCUCUUACAAAAAUGCU | 9886 | AGCAUUUUUGUAAGAGCAU |
| siRNA 4397 | 4397 | UGCUCUUACAAAAAUGCUA | 9887 | UAGCAUUUUUGUAAGAGCA |
| siRNA 4398 | 4398 | GCUCUUACAAAAAUGCUAC | 9888 | GUAGCAUUUUUGUAAGAGC |
| siRNA 4399 | 4399 | CUCUUACAAAAAUGCUACU | 9889 | AGUAGCAUUUUUGUAAGAG |
| siRNA 4400 | 4400 | UCUUACAAAAAUGCUACUG | 9890 | CAGUAGCAUUUUUGUAAGA |
| siRNA 4401 | 4401 | CUUACAAAAAUGCUACUGA | 9891 | UCAGUAGCAUUUUUGUAAG |
| siRNA 4402 | 4402 | UUACAAAAAUGCUACUGAU | 9892 | AUCAGUAGCAUUUUUGUAA |
| siRNA 4403 | 4403 | UACAAAAAUGCUACUGAUC | 9893 | GAUCAGUAGCAUUUUUGUA |
| siRNA 4404 | 4404 | ACAAAAAUGCUACUGAUCA | 9894 | UGAUCAGUAGCAUUUUUGU |
| siRNA 4405 | 4405 | CAAAAAUGCUACUGAUCAA | 9895 | UUGAUCAGUAGCAUUUUUG |
| siRNA 4406 | 4406 | AAAAAUGCUACUGAUCAAA | 9896 | UUUGAUCAGUAGCAUUUUU |
| siRNA 4407 | 4407 | AAAAUGCUACUGAUCAAAU | 9897 | AUUUGAUCAGUAGCAUUUU |
| siRNA 4408 | 4408 | AAAUGCUACUGAUCAAAUA | 9898 | UAUUUGAUCAGUAGCAUUU |
| siRNA 4409 | 4409 | AAUGCUACUGAUCAAAUAA | 9899 | UUAUUUGAUCAGUAGCAUU |
| siRNA 4410 | 4410 | AUGCUACUGAUCAAAUAAG | 9900 | CUUAUUUGAUCAGUAGCAU |
| siRNA 4411 | 4411 | UGCUACUGAUCAAAUAAGC | 9901 | GCUUAUUUGAUCAGUAGCA |
| siRNA 4412 | 4412 | GCUACUGAUCAAAUAAGCU | 9902 | AGCUUAUUUGAUCAGUAGC |
| siRNA 4413 | 4413 | CUACUGAUCAAAUAAGCUU | 9903 | AAGCUUAUUUGAUCAGUAG |
| siRNA 4414 | 4414 | UACUGAUCAAAUAAGCUUU | 9904 | AAAGCUUAUUUGAUCAGUA |
| siRNA 4415 | 4415 | ACUGAUCAAAUAAGCUUUU | 9905 | AAAAGCUUAUUUGAUCAGU |
| siRNA 4416 | 4416 | CUGAUCAAAUAAGCUUUUA | 9906 | UAAAAGCUUAUUUGAUCAG |
| siRNA 4417 | 4417 | UGAUCAAAUAAGCUUUUAA | 9907 | UUAAAAGCUUAUUUGAUCA |
| siRNA 4418 | 4418 | GAUCAAAUAAGCUUUUAAG | 9908 | CUUAAAAGCUUAUUUGAUC |
| siRNA 4419 | 4419 | AUCAAAUAAGCUUUUAAGA | 9909 | UCUUAAAAGCUUAUUUGAU |
| siRNA 4420 | 4420 | UCAAAUAAGCUUUUAAGAA | 9910 | UUCUUAAAAGCUUAUUUGA |
| siRNA 4421 | 4421 | CAAAUAAGCUUUUAAGAAU | 9911 | AUUCUUAAAAGCUUAUUUG |
| siRNA 4422 | 4422 | AAAUAAGCUUUUAAGAAUU | 9912 | AAUUCUUAAAAGCUUAUUU |
| siRNA 4423 | 4423 | AAUAAGCUUUUAAGAAUUG | 9913 | CAAUUCUUAAAAGCUUAUU |
| siRNA 4424 | 4424 | AUAAGCUUUUAAGAAUUGG | 9914 | CCAAUUCUUAAAAGCUUAU |
| siRNA 4425 | 4425 | UAAGCUUUUAAGAAUUGGA | 9915 | UCCAAUUCUUAAAAGCUUA |
| siRNA 4426 | 4426 | AAGCUUUUAAGAAUUGGAA | 9916 | UUCCAAUUCUUAAAAGCUU |
| siRNA 4427 | 4427 | AGCUUUUAAGAAUUGGAAA | 9917 | UUUCCAAUUCUUAAAAGCU |
| siRNA 4428 | 4428 | GCUUUUAAGAAUUGGAAAA | 9918 | UUUUCCAAUUCUUAAAAGC |
| siRNA 4429 | 4429 | CUUUUAAGAAUUGGAAAAA | 9919 | UUUUUCCAAUUCUUAAAAG |
| siRNA 4430 | 4430 | UUUUAAGAAUUGGAAAAAU | 9920 | AUUUUUCCAAUUCUUAAAA |
| siRNA 4431 | 4431 | UUUAAGAAUUGGAAAAAUA | 9921 | UAUUUUUCCAAUUCUUAAA |
| siRNA 4432 | 4432 | UUAAGAAUUGGAAAAAUAA | 9922 | UUAUUUUUCCAAUUCUUAA |
| siRNA 4433 | 4433 | UAAGAAUUGGAAAAAUAAA | 9923 | UUUAUUUUUCCAAUUCUUA |
| siRNA 4434 | 4434 | AAGAAUUGGAAAAAUAAAA | 9924 | UUUUAUUUUUCCAAUUCUU |
| siRNA 4435 | 4435 | AGAAUUGGAAAAAUAAAAA | 9925 | UUUUUAUUUUUCCAAUUCU |
| siRNA 4436 | 4436 | GAAUUGGAAAAAUAAAAAU | 9926 | AUUUUUAUUUUUCCAAUUC |
| siRNA 4437 | 4437 | AAUUGGAAAAAUAAAAAUA | 9927 | UAUUUUUAUUUUUCCAAUU |
| siRNA 4438 | 4438 | AUUGGAAAAAUAAAAAUAC | 9928 | GUAUUUUUAUUUUUCCAAU |
| siRNA 4439 | 4439 | UUGGAAAAAUAAAAAUACA | 9929 | UGUAUUUUUAUUUUUCCAA |
| siRNA 4440 | 4440 | UGGAAAAAUAAAAAUACAA | 9930 | UUGUAUUUUUAUUUUUCCA |
| siRNA 4441 | 4441 | GGAAAAAUAAAAAUACAAA | 9931 | UUUGUAUUUUUAUUUUUCC |
| siRNA 4442 | 4442 | GAAAAAUAAAAAUACAAAA | 9932 | UUUUGUAUUUUUAUUUUUC |
| siRNA 4443 | 4443 | AAAAAUAAAAAUACAAAAG | 9933 | CUUUUGUAUUUUUAUUUUU |
| siRNA 4444 | 4444 | AAAAUAAAAAUACAAAAGU | 9934 | ACUUUUGUAUUUUUAUUUU |
| siRNA 4445 | 4445 | AAAUAAAAAUACAAAAGUC | 9935 | GACUUUUGUAUUUUUAUUU |
| siRNA 4446 | 4446 | AAUAAAAAUACAAAAGUCC | 9936 | GGACUUUUGUAUUUUUAUU |
| siRNA 4447 | 4447 | AUAAAAAUACAAAAGUCCU | 9937 | AGGACUUUUGUAUUUUUAU |
| siRNA 4448 | 4448 | UAAAAAUACAAAAGUCCUU | 9938 | AAGGACUUUUGUAUUUUUA |
| siRNA 4449 | 4449 | AAAAAUACAAAAGUCCUUC | 9939 | GAAGGACUUUUGUAUUUUU |
| siRNA 4450 | 4450 | AAAAUACAAAAGUCCUUCA | 9940 | UGAAGGACUUUUGUAUUUU |
| siRNA 4451 | 4451 | AAAUACAAAAGUCCUUCAC | 9941 | GUGAAGGACUUUUGUAUUU |
| siRNA 4452 | 4452 | AAUACAAAAGUCCUUCACC | 9942 | GGUGAAGGACUUUUGUAUU |
| siRNA 4453 | 4453 | AUACAAAAGUCCUUCACCA | 9943 | UGGUGAAGGACUUUUGUAU |
| siRNA 4454 | 4454 | UACAAAAGUCCUUCACCAU | 9944 | AUGGUGAAGGACUUUUGUA |
| siRNA 4455 | 4455 | ACAAAAGUCCUUCACCAUU | 9945 | AAUGGUGAAGGACUUUUGU |
| siRNA 4456 | 4456 | CAAAAGUCCUUCACCAUUA | 9946 | UAAUGGUGAAGGACUUUUG |
| siRNA 4457 | 4457 | AAAAGUCCUUCACCAUUAU | 9947 | AUAAUGGUGAAGGACUUUU |
| siRNA 4458 | 4458 | AAAGUCCUUCACCAUUAUC | 9948 | GAUAAUGGUGAAGGACUUU |
| siRNA 4459 | 4459 | AAGUCCUUCACCAUUAUCC | 9949 | GGAUAAUGGUGAAGGACUU |
| siRNA 4460 | 4460 | AGUCCUUCACCAUUAUCCU | 9950 | AGGAUAAUGGUGAAGGACU |
| siRNA 4461 | 4461 | GUCCUUCACCAUUAUCCUC | 9951 | GAGGAUAAUGGUGAAGGAC |
| siRNA 4462 | 4462 | UCCUUCACCAUUAUCCUCA | 9952 | UGAGGAUAAUGGUGAAGGA |
| siRNA 4463 | 4463 | CCUUCACCAUUAUCCUCAG | 9953 | CUGAGGAUAAUGGUGAAGG |
| siRNA 4464 | 4464 | CUUCACCAUUAUCCUCAGG | 9954 | CCUGAGGAUAAUGGUGAAG |
| siRNA 4465 | 4465 | UUCACCAUUAUCCUCAGGU | 9955 | ACCUGAGGAUAAUGGUGAA |
| siRNA 4466 | 4466 | UCACCAUUAUCCUCAGGUG | 9956 | CACCUGAGGAUAAUGGUGA |
| siRNA 4467 | 4467 | CACCAUUAUCCUCAGGUGA | 9957 | UCACCUGAGGAUAAUGGUG |
| siRNA 4468 | 4468 | ACCAUUAUCCUCAGGUGAU | 9958 | AUCACCUGAGGAUAAUGGU |
| siRNA 4469 | 4469 | CCAUUAUCCUCAGGUGAUC | 9959 | GAUCACCUGAGGAUAAUGG |
| siRNA 4470 | 4470 | CAUUAUCCUCAGGUGAUCA | 9960 | UGAUCACCUGAGGAUAAUG |
| siRNA 4471 | 4471 | AUUAUCCUCAGGUGAUCAG | 9961 | CUGAUCACCUGAGGAUAAU |
| siRNA 4472 | 4472 | UUAUCCUCAGGUGAUCAGU | 9962 | ACUGAUCACCUGAGGAUAA |
| siRNA 4473 | 4473 | UAUCCUCAGGUGAUCAGUA | 9963 | UACUGAUCACCUGAGGAUA |
| siRNA 4474 | 4474 | AUCCUCAGGUGAUCAGUAG | 9964 | CUACUGAUCACCUGAGGAU |
| siRNA 4475 | 4475 | UCCUCAGGUGAUCAGUAGC | 9965 | GCUACUGAUCACCUGAGGA |
| siRNA 4476 | 4476 | CCUCAGGUGAUCAGUAGCA | 9966 | UGCUACUGAUCACCUGAGG |
| siRNA 4477 | 4477 | CUCAGGUGAUCAGUAGCAU | 9967 | AUGCUACUGAUCACCUGAG |
| siRNA 4478 | 4478 | UCAGGUGAUCAGUAGCAUU | 9968 | AAUGCUACUGAUCACCUGA |
| siRNA 4479 | 4479 | CAGGUGAUCAGUAGCAUUG | 9969 | CAAUGCUACUGAUCACCUG |
| siRNA 4480 | 4480 | AGGUGAUCAGUAGCAUUGC | 9970 | GCAAUGCUACUGAUCACCU |
| siRNA 4481 | 4481 | GGUGAUCAGUAGCAUUGCC | 9971 | GGCAAUGCUACUGAUCACC |
| siRNA 4482 | 4482 | GUGAUCAGUAGCAUUGCCU | 9972 | AGGCAAUGCUACUGAUCAC |
| siRNA 4483 | 4483 | UGAUCAGUAGCAUUGCCUU | 9973 | AAGGCAAUGCUACUGAUCA |
| siRNA 4484 | 4484 | GAUCAGUAGCAUUGCCUUG | 9974 | CAAGGCAAUGCUACUGAUC |
| siRNA 4485 | 4485 | AUCAGUAGCAUUGCCUUGU | 9975 | ACAAGGCAAUGCUACUGAU |
| siRNA 4486 | 4486 | UCAGUAGCAUUGCCUUGUC | 9976 | GACAAGGCAAUGCUACUGA |
| siRNA 4487 | 4487 | CAGUAGCAUUGCCUUGUCU | 9977 | AGACAAGGCAAUGCUACUG |
| siRNA 4488 | 4488 | AGUAGCAUUGCCUUGUCUU | 9978 | AAGACAAGGCAAUGCUACU |
| siRNA 4489 | 4489 | GUAGCAUUGCCUUGUCUUG | 9979 | CAAGACAAGGCAAUGCUAC |
| siRNA 4490 | 4490 | UAGCAUUGCCUUGUCUUGG | 9980 | CCAAGACAAGGCAAUGCUA |
| siRNA 4491 | 4491 | AGCAUUGCCUUGUCUUGGA | 9981 | UCCAAGACAAGGCAAUGCU |
| siRNA 4492 | 4492 | GCAUUGCCUUGUCUUGGAU | 9982 | AUCCAAGACAAGGCAAUGC |
| siRNA 4493 | 4493 | CAUUGCCUUGUCUUGGAUC | 9983 | GAUCCAAGACAAGGCAAUG |
| siRNA 4494 | 4494 | AUUGCCUUGUCUUGGAUCC | 9984 | GGAUCCAAGACAAGGCAAU |
| siRNA 4495 | 4495 | UUGCCUUGUCUUGGAUCCU | 9985 | AGGAUCCAAGACAAGGCAA |
| siRNA 4496 | 4496 | UGCCUUGUCUUGGAUCCUC | 9986 | GAGGAUCCAAGACAAGGCA |
| siRNA 4497 | 4497 | GCCUUGUCUUGGAUCCUCA | 9987 | UGAGGAUCCAAGACAAGGC |
| siRNA 4498 | 4498 | CCUUGUCUUGGAUCCUCAG | 9988 | CUGAGGAUCCAAGACAAGG |
| siRNA 4499 | 4499 | CUUGUCUUGGAUCCUCAGU | 9989 | ACUGAGGAUCCAAGACAAG |
| siRNA 4500 | 4500 | UUGUCUUGGAUCCUCAGUG | 9990 | CACUGAGGAUCCAAGACAA |
| siRNA 4501 | 4501 | UGUCUUGGAUCCUCAGUGC | 9991 | GCACUGAGGAUCCAAGACA |
| siRNA 4502 | 4502 | GUCUUGGAUCCUCAGUGCU | 9992 | AGCACUGAGGAUCCAAGAC |
| siRNA 4503 | 4503 | UCUUGGAUCCUCAGUGCUG | 9993 | CAGCACUGAGGAUCCAAGA |
| siRNA 4504 | 4504 | CUUGGAUCCUCAGUGCUGC | 9994 | GCAGCACUGAGGAUCCAAG |
| siRNA 4505 | 4505 | UUGGAUCCUCAGUGCUGCC | 9995 | GGCAGCACUGAGGAUCCAA |
| siRNA 4506 | 4506 | UGGAUCCUCAGUGCUGCCA | 9996 | UGGCAGCACUGAGGAUCCA |
| siRNA 4507 | 4507 | GGAUCCUCAGUGCUGCCAA | 9997 | UUGGCAGCACUGAGGAUCC |
| siRNA 4508 | 4508 | GAUCCUCAGUGCUGCCAAA | 9998 | UUUGGCAGCACUGAGGAUC |
| siRNA 4509 | 4509 | AUCCUCAGUGCUGCCAAAA | 9999 | UUUUGGCAGCACUGAGGAU |
| siRNA 4510 | 4510 | UCCUCAGUGCUGCCAAAAG | 10000 | CUUUUGGCAGCACUGAGGA |
| siRNA 4511 | 4511 | CCUCAGUGCUGCCAAAAGG | 10001 | CCUUUUGGCAGCACUGAGG |
| siRNA 4512 | 4512 | CUCAGUGCUGCCAAAAGGC | 10002 | GCCUUUUGGCAGCACUGAG |
| siRNA 4513 | 4513 | UCAGUGCUGCCAAAAGGCC | 10003 | GGCCUUUUGGCAGCACUGA |
| siRNA 4514 | 4514 | CAGUGCUGCCAAAAGGCCA | 10004 | UGGCCUUUUGGCAGCACUG |
| siRNA 4515 | 4515 | AGUGCUGCCAAAAGGCCAG | 10005 | CUGGCCUUUUGGCAGCACU |
| siRNA 4516 | 4516 | GUGCUGCCAAAAGGCCAGU | 10006 | ACUGGCCUUUUGGCAGCAC |
| siRNA 4517 | 4517 | UGCUGCCAAAAGGCCAGUA | 10007 | UACUGGCCUUUUGGCAGCA |
| siRNA 4518 | 4518 | GCUGCCAAAAGGCCAGUAU | 10008 | AUACUGGCCUUUUGGCAGC |
| siRNA 4519 | 4519 | CUGCCAAAAGGCCAGUAUA | 10009 | UAUACUGGCCUUUUGGCAG |
| siRNA 4520 | 4520 | UGCCAAAAGGCCAGUAUAA | 10010 | UUAUACUGGCCUUUUGGCA |
| siRNA 4521 | 4521 | GCCAAAAGGCCAGUAUAAA | 10011 | UUUAUACUGGCCUUUUGGC |
| siRNA 4522 | 4522 | CCAAAAGGCCAGUAUAAAG | 10012 | CUUUAUACUGGCCUUUUGG |
| siRNA 4523 | 4523 | CAAAAGGCCAGUAUAAAGA | 10013 | UCUUUAUACUGGCCUUUUG |
| siRNA 4524 | 4524 | AAAAGGCCAGUAUAAAGAA | 10014 | UUCUUUAUACUGGCCUUUU |
| siRNA 4525 | 4525 | AAAGGCCAGUAUAAAGAAU | 10015 | AUUCUUUAUACUGGCCUUU |
| siRNA 4526 | 4526 | AAGGCCAGUAUAAAGAAUU | 10016 | AAUUCUUUAUACUGGCCUU |
| siRNA 4527 | 4527 | AGGCCAGUAUAAAGAAUUU | 10017 | AAAUUCUUUAUACUGGCCU |
| siRNA 4528 | 4528 | GGCCAGUAUAAAGAAUUUA | 10018 | UAAAUUCUUUAUACUGGCC |
| siRNA 4529 | 4529 | GCCAGUAUAAAGAAUUUAU | 10019 | AUAAAUUCUUUAUACUGGC |
| siRNA 4530 | 4530 | CCAGUAUAAAGAAUUUAUA | 10020 | UAUAAAUUCUUUAUACUGG |
| siRNA 4531 | 4531 | CAGUAUAAAGAAUUUAUAU | 10021 | AUAUAAAUUCUUUAUACUG |
| siRNA 4532 | 4532 | AGUAUAAAGAAUUUAUAUU | 10022 | AAUAUAAAUUCUUUAUACU |
| siRNA 4533 | 4533 | GUAUAAAGAAUUUAUAUUU | 10023 | AAAUAUAAAUUCUUUAUAC |
| siRNA 4534 | 4534 | UAUAAAGAAUUUAUAUUUG | 10024 | CAAAUAUAAAUUCUUUAUA |
| siRNA 4535 | 4535 | AUAAAGAAUUUAUAUUUGC | 10025 | GCAAAUAUAAAUUCUUUAU |
| siRNA 4536 | 4536 | UAAAGAAUUUAUAUUUGCA | 10026 | UGCAAAUAUAAAUUCUUUA |
| siRNA 4537 | 4537 | AAAGAAUUUAUAUUUGCAC | 10027 | GUGCAAAUAUAAAUUCUUU |
| siRNA 4538 | 4538 | AAGAAUUUAUAUUUGCACU | 10028 | AGUGCAAAUAUAAAUUCUU |
| siRNA 4539 | 4539 | AGAAUUUAUAUUUGCACUG | 10029 | CAGUGCAAAUAUAAAUUCU |
| siRNA 4540 | 4540 | GAAUUUAUAUUUGCACUGU | 10030 | ACAGUGCAAAUAUAAAUUC |
| siRNA 4541 | 4541 | AAUUUAUAUUUGCACUGUA | 10031 | UACAGUGCAAAUAUAAAUU |
| siRNA 4542 | 4542 | AUUUAUAUUUGCACUGUAA | 10032 | UUACAGUGCAAAUAUAAAU |
| siRNA 4543 | 4543 | UUUAUAUUUGCACUGUAAA | 10033 | UUUACAGUGCAAAUAUAAA |
| siRNA 4544 | 4544 | UUAUAUUUGCACUGUAAAC | 10034 | GUUUACAGUGCAAAUAUAA |
| siRNA 4545 | 4545 | UAUAUUUGCACUGUAAACU | 10035 | AGUUUACAGUGCAAAUAUA |
| siRNA 4546 | 4546 | AUAUUUGCACUGUAAACUC | 10036 | GAGUUUACAGUGCAAAUAU |
| siRNA 4547 | 4547 | UAUUUGCACUGUAAACUCU | 10037 | AGAGUUUACAGUGCAAAUA |
| siRNA 4548 | 4548 | AUUUGCACUGUAAACUCUG | 10038 | CAGAGUUUACAGUGCAAAU |
| siRNA 4549 | 4549 | UUUGCACUGUAAACUCUGC | 10039 | GCAGAGUUUACAGUGCAAA |
| siRNA 4550 | 4550 | UUGCACUGUAAACUCUGCA | 10040 | UGCAGAGUUUACAGUGCAA |
| siRNA 4551 | 4551 | UGCACUGUAAACUCUGCAA | 10041 | UUGCAGAGUUUACAGUGCA |
| siRNA 4552 | 4552 | GCACUGUAAACUCUGCAAA | 10042 | UUUGCAGAGUUUACAGUGC |
| siRNA 4553 | 4553 | CACUGUAAACUCUGCAAAA | 10043 | UUUUGCAGAGUUUACAGUG |
| siRNA 4554 | 4554 | ACUGUAAACUCUGCAAAAA | 10044 | UUUUUGCAGAGUUUACAGU |
| siRNA 4555 | 4555 | CUGUAAACUCUGCAAAAAU | 10045 | AUUUUUGCAGAGUUUACAG |
| siRNA 4556 | 4556 | UGUAAACUCUGCAAAAAUA | 10046 | UAUUUUUGCAGAGUUUACA |
| siRNA 4557 | 4557 | GUAAACUCUGCAAAAAUAU | 10047 | AUAUUUUUGCAGAGUUUAC |
| siRNA 4558 | 4558 | UAAACUCUGCAAAAAUAUG | 10048 | CAUAUUUUUGCAGAGUUUA |
| siRNA 4559 | 4559 | AAACUCUGCAAAAAUAUGG | 10049 | CCAUAUUUUUGCAGAGUUU |
| siRNA 4560 | 4560 | AACUCUGCAAAAAUAUGGU | 10050 | ACCAUAUUUUUGCAGAGUU |
| siRNA 4561 | 4561 | ACUCUGCAAAAAUAUGGUU | 10051 | AACCAUAUUUUUGCAGAGU |
| siRNA 4562 | 4562 | CUCUGCAAAAAUAUGGUUU | 10052 | AAACCAUAUUUUUGCAGAG |
| siRNA 4563 | 4563 | UCUGCAAAAAUAUGGUUUA | 10053 | UAAACCAUAUUUUUGCAGA |
| siRNA 4564 | 4564 | CUGCAAAAAUAUGGUUUAA | 10054 | UUAAACCAUAUUUUUGCAG |
| siRNA 4565 | 4565 | UGCAAAAAUAUGGUUUAAA | 10055 | UUUAAACCAUAUUUUUGCA |
| siRNA 4566 | 4566 | GCAAAAAUAUGGUUUAAAG | 10056 | CUUUAAACCAUAUUUUUGC |
| siRNA 4567 | 4567 | CAAAAAUAUGGUUUAAAGU | 10057 | ACUUUAAACCAUAUUUUUG |
| siRNA 4568 | 4568 | AAAAAUAUGGUUUAAAGUG | 10058 | CACUUUAAACCAUAUUUUU |
| siRNA 4569 | 4569 | AAAAUAUGGUUUAAAGUGA | 10059 | UCACUUUAAACCAUAUUUU |
| siRNA 4570 | 4570 | AAAUAUGGUUUAAAGUGAC | 10060 | GUCACUUUAAACCAUAUUU |
| siRNA 4571 | 4571 | AAUAUGGUUUAAAGUGACA | 10061 | UGUCACUUUAAACCAUAUU |
| siRNA 4572 | 4572 | AUAUGGUUUAAAGUGACAU | 10062 | AUGUCACUUUAAACCAUAU |
| siRNA 4573 | 4573 | UAUGGUUUAAAGUGACAUG | 10063 | CAUGUCACUUUAAACCAUA |
| siRNA 4574 | 4574 | AUGGUUUAAAGUGACAUGA | 10064 | UCAUGUCACUUUAAACCAU |
| siRNA 4575 | 4575 | UGGUUUAAAGUGACAUGAU | 10065 | AUCAUGUCACUUUAAACCA |
| siRNA 4576 | 4576 | GGUUUAAAGUGACAUGAUU | 10066 | AAUCAUGUCACUUUAAACC |
| siRNA 4577 | 4577 | GUUUAAAGUGACAUGAUUG | 10067 | CAAUCAUGUCACUUUAAAC |
| siRNA 4578 | 4578 | UUUAAAGUGACAUGAUUGC | 10068 | GCAAUCAUGUCACUUUAAA |
| siRNA 4579 | 4579 | UUAAAGUGACAUGAUUGCA | 10069 | UGCAAUCAUGUCACUUUAA |
| siRNA 4580 | 4580 | UAAAGUGACAUGAUUGCAC | 10070 | GUGCAAUCAUGUCACUUUA |
| siRNA 4581 | 4581 | AAAGUGACAUGAUUGCACU | 10071 | AGUGCAAUCAUGUCACUUU |
| siRNA 4582 | 4582 | AAGUGACAUGAUUGCACUG | 10072 | CAGUGCAAUCAUGUCACUU |
| siRNA 4583 | 4583 | AGUGACAUGAUUGCACUGA | 10073 | UCAGUGCAAUCAUGUCACU |
| siRNA 4584 | 4584 | GUGACAUGAUUGCACUGAA | 10074 | UUCAGUGCAAUCAUGUCAC |
| siRNA 4585 | 4585 | UGACAUGAUUGCACUGAAA | 10075 | UUUCAGUGCAAUCAUGUCA |
| siRNA 4586 | 4586 | GACAUGAUUGCACUGAAAA | 10076 | UUUUCAGUGCAAUCAUGUC |
| siRNA 4587 | 4587 | ACAUGAUUGCACUGAAAAG | 10077 | CUUUUCAGUGCAAUCAUGU |
| siRNA 4588 | 4588 | CAUGAUUGCACUGAAAAGG | 10078 | CCUUUUCAGUGCAAUCAUG |
| siRNA 4589 | 4589 | AUGAUUGCACUGAAAAGGG | 10079 | CCCUUUUCAGUGCAAUCAU |
| siRNA 4590 | 4590 | UGAUUGCACUGAAAAGGGA | 10080 | UCCCUUUUCAGUGCAAUCA |
| siRNA 4591 | 4591 | GAUUGCACUGAAAAGGGAU | 10081 | AUCCCUUUUCAGUGCAAUC |
| siRNA 4592 | 4592 | AUUGCACUGAAAAGGGAUA | 10082 | UAUCCCUUUUCAGUGCAAU |
| siRNA 4593 | 4593 | UUGCACUGAAAAGGGAUAG | 10083 | CUAUCCCUUUUCAGUGCAA |
| siRNA 4594 | 4594 | UGCACUGAAAAGGGAUAGU | 10084 | ACUAUCCCUUUUCAGUGCA |
| siRNA 4595 | 4595 | GCACUGAAAAGGGAUAGUG | 10085 | CACUAUCCCUUUUCAGUGC |
| siRNA 4596 | 4596 | CACUGAAAAGGGAUAGUGC | 10086 | GCACUAUCCCUUUUCAGUG |
| siRNA 4597 | 4597 | ACUGAAAAGGGAUAGUGCU | 10087 | AGCACUAUCCCUUUUCAGU |
| siRNA 4598 | 4598 | CUGAAAAGGGAUAGUGCUU | 10088 | AAGCACUAUCCCUUUUCAG |
| siRNA 4599 | 4599 | UGAAAAGGGAUAGUGCUUU | 10089 | AAAGCACUAUCCCUUUUCA |
| siRNA 4600 | 4600 | GAAAAGGGAUAGUGCUUUU | 10090 | AAAAGCACUAUCCCUUUUC |
| siRNA 4601 | 4601 | AAAAGGGAUAGUGCUUUUG | 10091 | CAAAAGCACUAUCCCUUUU |
| siRNA 4602 | 4602 | AAAGGGAUAGUGCUUUUGU | 10092 | ACAAAAGCACUAUCCCUUU |
| siRNA 4603 | 4603 | AAGGGAUAGUGCUUUUGUG | 10093 | CACAAAAGCACUAUCCCUU |
| siRNA 4604 | 4604 | AGGGAUAGUGCUUUUGUGA | 10094 | UCACAAAAGCACUAUCCCU |
| siRNA 4605 | 4605 | GGGAUAGUGCUUUUGUGAA | 10095 | UUCACAAAAGCACUAUCCC |
| siRNA 4606 | 4606 | GGAUAGUGCUUUUGUGAAA | 10096 | UUUCACAAAAGCACUAUCC |
| siRNA 4607 | 4607 | GAUAGUGCUUUUGUGAAAU | 10097 | AUUUCACAAAAGCACUAUC |
| siRNA 4608 | 4608 | AUAGUGCUUUUGUGAAAUU | 10098 | AAUUUCACAAAAGCACUAU |
| siRNA 4609 | 4609 | UAGUGCUUUUGUGAAAUUU | 10099 | AAAUUUCACAAAAGCACUA |
| siRNA 4610 | 4610 | AGUGCUUUUGUGAAAUUUU | 10100 | AAAAUUUCACAAAAGCACU |
| siRNA 4611 | 4611 | GUGCUUUUGUGAAAUUUUU | 10101 | AAAAAUUUCACAAAAGCAC |
| siRNA 4612 | 4612 | UGCUUUUGUGAAAUUUUUC | 10102 | GAAAAAUUUCACAAAAGCA |
| siRNA 4613 | 4613 | GCUUUUGUGAAAUUUUUCA | 10103 | UGAAAAAUUUCACAAAAGC |
| siRNA 4614 | 4614 | CUUUUGUGAAAUUUUUCAA | 10104 | UUGAAAAAUUUCACAAAAG |
| siRNA 4615 | 4615 | UUUUGUGAAAUUUUUCAAA | 10105 | UUUGAAAAAUUUCACAAAA |
| siRNA 4616 | 4616 | UUUGUGAAAUUUUUCAAAU | 10106 | AUUUGAAAAAUUUCACAAA |
| siRNA 4617 | 4617 | UUGUGAAAUUUUUCAAAUU | 10107 | AAUUUGAAAAAUUUCACAA |
| siRNA 4618 | 4618 | UGUGAAAUUUUUCAAAUUU | 10108 | AAAUUUGAAAAAUUUCACA |
| siRNA 4619 | 4619 | GUGAAAUUUUUCAAAUUUG | 10109 | CAAAUUUGAAAAAUUUCAC |
| siRNA 4620 | 4620 | UGAAAUUUUUCAAAUUUGA | 10110 | UCAAAUUUGAAAAAUUUCA |
| siRNA 4621 | 4621 | GAAAUUUUUCAAAUUUGAG | 10111 | CUCAAAUUUGAAAAAUUUC |
| siRNA 4622 | 4622 | AAAUUUUUCAAAUUUGAGU | 10112 | ACUCAAAUUUGAAAAAUUU |
| siRNA 4623 | 4623 | AAUUUUUCAAAUUUGAGUA | 10113 | UACUCAAAUUUGAAAAAUU |
| siRNA 4624 | 4624 | AUUUUUCAAAUUUGAGUAA | 10114 | UUACUCAAAUUUGAAAAAU |
| siRNA 4625 | 4625 | UUUUUCAAAUUUGAGUAAU | 10115 | AUUACUCAAAUUUGAAAAA |
| siRNA 4626 | 4626 | UUUUCAAAUUUGAGUAAUA | 10116 | UAUUACUCAAAUUUGAAAA |
| siRNA 4627 | 4627 | UUUCAAAUUUGAGUAAUAG | 10117 | CUAUUACUCAAAUUUGAAA |
| siRNA 4628 | 4628 | UUCAAAUUUGAGUAAUAGA | 10118 | UCUAUUACUCAAAUUUGAA |
| siRNA 4629 | 4629 | UCAAAUUUGAGUAAUAGAU | 10119 | AUCUAUUACUCAAAUUUGA |
| siRNA 4630 | 4630 | CAAAUUUGAGUAAUAGAUG | 10120 | CAUCUAUUACUCAAAUUUG |
| siRNA 4631 | 4631 | AAAUUUGAGUAAUAGAUGC | 10121 | GCAUCUAUUACUCAAAUUU |
| siRNA 4632 | 4632 | AAUUUGAGUAAUAGAUGCC | 10122 | GGCAUCUAUUACUCAAAUU |
| siRNA 4633 | 4633 | AUUUGAGUAAUAGAUGCCU | 10123 | AGGCAUCUAUUACUCAAAU |
| siRNA 4634 | 4634 | UUUGAGUAAUAGAUGCCUU | 10124 | AAGGCAUCUAUUACUCAAA |
| siRNA 4635 | 4635 | UUGAGUAAUAGAUGCCUUU | 10125 | AAAGGCAUCUAUUACUCAA |
| siRNA 4636 | 4636 | UGAGUAAUAGAUGCCUUUU | 10126 | AAAAGGCAUCUAUUACUCA |
| siRNA 4637 | 4637 | GAGUAAUAGAUGCCUUUUU | 10127 | AAAAAGGCAUCUAUUACUC |
| siRNA 4638 | 4638 | AGUAAUAGAUGCCUUUUUA | 10128 | UAAAAAGGCAUCUAUUACU |
| siRNA 4639 | 4639 | GUAAUAGAUGCCUUUUUAA | 10129 | UUAAAAAGGCAUCUAUUAC |
| siRNA 4640 | 4640 | UAAUAGAUGCCUUUUUAAG | 10130 | CUUAAAAAGGCAUCUAUUA |
| siRNA 4641 | 4641 | AAUAGAUGCCUUUUUAAGG | 10131 | CCUUAAAAAGGCAUCUAUU |
| siRNA 4642 | 4642 | AUAGAUGCCUUUUUAAGGC | 10132 | GCCUUAAAAAGGCAUCUAU |
| siRNA 4643 | 4643 | UAGAUGCCUUUUUAAGGCA | 10133 | UGCCUUAAAAAGGCAUCUA |
| siRNA 4644 | 4644 | AGAUGCCUUUUUAAGGCAG | 10134 | CUGCCUUAAAAAGGCAUCU |
| siRNA 4645 | 4645 | GAUGCCUUUUUAAGGCAGU | 10135 | ACUGCCUUAAAAAGGCAUC |
| siRNA 4646 | 4646 | AUGCCUUUUUAAGGCAGUG | 10136 | CACUGCCUUAAAAAGGCAU |
| siRNA 4647 | 4647 | UGCCUUUUUAAGGCAGUGA | 10137 | UCACUGCCUUAAAAAGGCA |
| siRNA 4648 | 4648 | GCCUUUUUAAGGCAGUGAA | 10138 | UUCACUGCCUUAAAAAGGC |
| siRNA 4649 | 4649 | CCUUUUUAAGGCAGUGAAU | 10139 | AUUCACUGCCUUAAAAAGG |
| siRNA 4650 | 4650 | CUUUUUAAGGCAGUGAAUU | 10140 | AAUUCACUGCCUUAAAAAG |
| siRNA 4651 | 4651 | UUUUUAAGGCAGUGAAUUU | 10141 | AAAUUCACUGCCUUAAAAA |
| siRNA 4652 | 4652 | UUUUAAGGCAGUGAAUUUA | 10142 | UAAAUUCACUGCCUUAAAA |
| siRNA 4653 | 4653 | UUUAAGGCAGUGAAUUUAC | 10143 | GUAAAUUCACUGCCUUAAA |
| siRNA 4654 | 4654 | UUAAGGCAGUGAAUUUACA | 10144 | UGUAAAUUCACUGCCUUAA |
| siRNA 4655 | 4655 | UAAGGCAGUGAAUUUACAC | 10145 | GUGUAAAUUCACUGCCUUA |
| siRNA 4656 | 4656 | AAGGCAGUGAAUUUACACA | 10146 | UGUGUAAAUUCACUGCCUU |
| siRNA 4657 | 4657 | AGGCAGUGAAUUUACACAA | 10147 | UUGUGUAAAUUCACUGCCU |
| siRNA 4658 | 4658 | GGCAGUGAAUUUACACAAA | 10148 | UUUGUGUAAAUUCACUGCC |
| siRNA 4659 | 4659 | GCAGUGAAUUUACACAAAU | 10149 | AUUUGUGUAAAUUCACUGC |
| siRNA 4660 | 4660 | CAGUGAAUUUACACAAAUA | 10150 | UAUUUGUGUAAAUUCACUG |
| siRNA 4661 | 4661 | AGUGAAUUUACACAAAUAU | 10151 | AUAUUUGUGUAAAUUCACU |
| siRNA 4662 | 4662 | GUGAAUUUACACAAAUAUG | 10152 | CAUAUUUGUGUAAAUUCAC |
| siRNA 4663 | 4663 | UGAAUUUACACAAAUAUGG | 10153 | CCAUAUUUGUGUAAAUUCA |
| siRNA 4664 | 4664 | GAAUUUACACAAAUAUGGG | 10154 | CCCAUAUUUGUGUAAAUUC |
| siRNA 4665 | 4665 | AAUUUACACAAAUAUGGGA | 10155 | UCCCAUAUUUGUGUAAAUU |
| siRNA 4666 | 4666 | AUUUACACAAAUAUGGGAG | 10156 | CUCCCAUAUUUGUGUAAAU |
| siRNA 4667 | 4667 | UUUACACAAAUAUGGGAGG | 10157 | CCUCCCAUAUUUGUGUAAA |
| siRNA 4668 | 4668 | UUACACAAAUAUGGGAGGG | 10158 | CCCUCCCAUAUUUGUGUAA |
| siRNA 4669 | 4669 | UACACAAAUAUGGGAGGGG | 10159 | CCCCUCCCAUAUUUGUGUA |
| siRNA 4670 | 4670 | ACACAAAUAUGGGAGGGGU | 10160 | ACCCCUCCCAUAUUUGUGU |
| siRNA 4671 | 4671 | CACAAAUAUGGGAGGGGUA | 10161 | UACCCCUCCCAUAUUUGUG |
| siRNA 4672 | 4672 | ACAAAUAUGGGAGGGGUAU | 10162 | AUACCCCUCCCAUAUUUGU |
| siRNA 4673 | 4673 | CAAAUAUGGGAGGGGUAUA | 10163 | UAUACCCCUCCCAUAUUUG |
| siRNA 4674 | 4674 | AAAUAUGGGAGGGGUAUAU | 10164 | AUAUACCCCUCCCAUAUUU |
| siRNA 4675 | 4675 | AAUAUGGGAGGGGUAUAUG | 10165 | CAUAUACCCCUCCCAUAUU |
| siRNA 4676 | 4676 | AUAUGGGAGGGGUAUAUGG | 10166 | CCAUAUACCCCUCCCAUAU |
| siRNA 4677 | 4677 | UAUGGGAGGGGUAUAUGGU | 10167 | ACCAUAUACCCCUCCCAUA |
| siRNA 4678 | 4678 | AUGGGAGGGGUAUAUGGUG | 10168 | CACCAUAUACCCCUCCCAU |
| siRNA 4679 | 4679 | UGGGAGGGGUAUAUGGUGU | 10169 | ACACCAUAUACCCCUCCCA |
| siRNA 4680 | 4680 | GGGAGGGGUAUAUGGUGUU | 10170 | AACACCAUAUACCCCUCCC |
| siRNA 4681 | 4681 | GGAGGGGUAUAUGGUGUUA | 10171 | UAACACCAUAUACCCCUCC |
| siRNA 4682 | 4682 | GAGGGGUAUAUGGUGUUAC | 10172 | GUAACACCAUAUACCCCUC |
| siRNA 4683 | 4683 | AGGGGUAUAUGGUGUUACU | 10173 | AGUAACACCAUAUACCCCU |
| siRNA 4684 | 4684 | GGGGUAUAUGGUGUUACUG | 10174 | CAGUAACACCAUAUACCCC |
| siRNA 4685 | 4685 | GGGUAUAUGGUGUUACUGA | 10175 | UCAGUAACACCAUAUACCC |
| siRNA 4686 | 4686 | GGUAUAUGGUGUUACUGAU | 10176 | AUCAGUAACACCAUAUACC |
| siRNA 4687 | 4687 | GUAUAUGGUGUUACUGAUU | 10177 | AAUCAGUAACACCAUAUAC |
| siRNA 4688 | 4688 | UAUAUGGUGUUACUGAUUU | 10178 | AAAUCAGUAACACCAUAUA |
| siRNA 4689 | 4689 | AUAUGGUGUUACUGAUUUU | 10179 | AAAAUCAGUAACACCAUAU |
| siRNA 4690 | 4690 | UAUGGUGUUACUGAUUUUU | 10180 | AAAAAUCAGUAACACCAUA |
| siRNA 4691 | 4691 | AUGGUGUUACUGAUUUUUA | 10181 | UAAAAAUCAGUAACACCAU |
| siRNA 4692 | 4692 | UGGUGUUACUGAUUUUUAA | 10182 | UUAAAAAUCAGUAACACCA |
| siRNA 4693 | 4693 | GGUGUUACUGAUUUUUAAA | 10183 | UUUAAAAAUCAGUAACACC |
| siRNA 4694 | 4694 | GUGUUACUGAUUUUUAAAC | 10184 | GUUUAAAAAUCAGUAACAC |
| siRNA 4695 | 4695 | UGUUACUGAUUUUUAAACC | 10185 | GGUUUAAAAAUCAGUAACA |
| siRNA 4696 | 4696 | GUUACUGAUUUUUAAACCU | 10186 | AGGUUUAAAAAUCAGUAAC |
| siRNA 4697 | 4697 | UUACUGAUUUUUAAACCUC | 10187 | GAGGUUUAAAAAUCAGUAA |
| siRNA 4698 | 4698 | UACUGAUUUUUAAACCUCU | 10188 | AGAGGUUUAAAAAUCAGUA |
| siRNA 4699 | 4699 | ACUGAUUUUUAAACCUCUU | 10189 | AAGAGGUUUAAAAAUCAGU |
| siRNA 4700 | 4700 | CUGAUUUUUAAACCUCUUU | 10190 | AAAGAGGUUUAAAAAUCAG |
| siRNA 4701 | 4701 | UGAUUUUUAAACCUCUUUG | 10191 | CAAAGAGGUUUAAAAAUCA |
| siRNA 4702 | 4702 | GAUUUUUAAACCUCUUUGA | 10192 | UCAAAGAGGUUUAAAAAUC |
| siRNA 4703 | 4703 | AUUUUUAAACCUCUUUGAC | 10193 | GUCAAAGAGGUUUAAAAAU |
| siRNA 4704 | 4704 | UUUUUAAACCUCUUUGACC | 10194 | GGUCAAAGAGGUUUAAAAA |
| siRNA 4705 | 4705 | UUUUAAACCUCUUUGACCA | 10195 | UGGUCAAAGAGGUUUAAAA |
| siRNA 4706 | 4706 | UUUAAACCUCUUUGACCAU | 10196 | AUGGUCAAAGAGGUUUAAA |
| siRNA 4707 | 4707 | UUAAACCUCUUUGACCAUC | 10197 | GAUGGUCAAAGAGGUUUAA |
| siRNA 4708 | 4708 | UAAACCUCUUUGACCAUCU | 10198 | AGAUGGUCAAAGAGGUUUA |
| siRNA 4709 | 4709 | AAACCUCUUUGACCAUCUU | 10199 | AAGAUGGUCAAAGAGGUUU |
| siRNA 4710 | 4710 | AACCUCUUUGACCAUCUUC | 10200 | GAAGAUGGUCAAAGAGGUU |
| siRNA 4711 | 4711 | ACCUCUUUGACCAUCUUCU | 10201 | AGAAGAUGGUCAAAGAGGU |
| siRNA 4712 | 4712 | CCUCUUUGACCAUCUUCUA | 10202 | UAGAAGAUGGUCAAAGAGG |
| siRNA 4713 | 4713 | CUCUUUGACCAUCUUCUAG | 10203 | CUAGAAGAUGGUCAAAGAG |
| siRNA 4714 | 4714 | UCUUUGACCAUCUUCUAGU | 10204 | ACUAGAAGAUGGUCAAAGA |
| siRNA 4715 | 4715 | CUUUGACCAUCUUCUAGUU | 10205 | AACUAGAAGAUGGUCAAAG |
| siRNA 4716 | 4716 | UUUGACCAUCUUCUAGUUU | 10206 | AAACUAGAAGAUGGUCAAA |
| siRNA 4717 | 4717 | UUGACCAUCUUCUAGUUUU | 10207 | AAAACUAGAAGAUGGUCAA |
| siRNA 4718 | 4718 | UGACCAUCUUCUAGUUUUU | 10208 | AAAAACUAGAAGAUGGUCA |
| siRNA 4719 | 4719 | GACCAUCUUCUAGUUUUUA | 10209 | UAAAAACUAGAAGAUGGUC |
| siRNA 4720 | 4720 | ACCAUCUUCUAGUUUUUAC | 10210 | GUAAAAACUAGAAGAUGGU |
| siRNA 4721 | 4721 | CCAUCUUCUAGUUUUUACU | 10211 | AGUAAAAACUAGAAGAUGG |
| siRNA 4722 | 4722 | CAUCUUCUAGUUUUUACUU | 10212 | AAGUAAAAACUAGAAGAUG |
| siRNA 4723 | 4723 | AUCUUCUAGUUUUUACUUC | 10213 | GAAGUAAAAACUAGAAGAU |
| siRNA 4724 | 4724 | UCUUCUAGUUUUUACUUCU | 10214 | AGAAGUAAAAACUAGAAGA |
| siRNA 4725 | 4725 | CUUCUAGUUUUUACUUCUA | 10215 | UAGAAGUAAAAACUAGAAG |
| siRNA 4726 | 4726 | UUCUAGUUUUUACUUCUAG | 10216 | CUAGAAGUAAAAACUAGAA |
| siRNA 4727 | 4727 | UCUAGUUUUUACUUCUAGU | 10217 | ACUAGAAGUAAAAACUAGA |
| siRNA 4728 | 4728 | CUAGUUUUUACUUCUAGUU | 10218 | AACUAGAAGUAAAAACUAG |
| siRNA 4729 | 4729 | UAGUUUUUACUUCUAGUUU | 10219 | AAACUAGAAGUAAAAACUA |
| siRNA 4730 | 4730 | AGUUUUUACUUCUAGUUUU | 10220 | AAAACUAGAAGUAAAAACU |
| siRNA 4731 | 4731 | GUUUUUACUUCUAGUUUUU | 10221 | AAAAACUAGAAGUAAAAAC |
| siRNA 4732 | 4732 | UUUUUACUUCUAGUUUUUA | 10222 | UAAAAACUAGAAGUAAAAA |
| siRNA 4733 | 4733 | UUUUACUUCUAGUUUUUAC | 10223 | GUAAAAACUAGAAGUAAAA |
| siRNA 4734 | 4734 | UUUACUUCUAGUUUUUACA | 10224 | UGUAAAAACUAGAAGUAAA |
| siRNA 4735 | 4735 | UUACUUCUAGUUUUUACAU | 10225 | AUGUAAAAACUAGAAGUAA |
| siRNA 4736 | 4736 | UACUUCUAGUUUUUACAUC | 10226 | GAUGUAAAAACUAGAAGUA |
| siRNA 4737 | 4737 | ACUUCUAGUUUUUACAUCU | 10227 | AGAUGUAAAAACUAGAAGU |
| siRNA 4738 | 4738 | CUUCUAGUUUUUACAUCUA | 10228 | UAGAUGUAAAAACUAGAAG |
| siRNA 4739 | 4739 | UUCUAGUUUUUACAUCUAG | 10229 | CUAGAUGUAAAAACUAGAA |
| siRNA 4740 | 4740 | UCUAGUUUUUACAUCUAGG | 10230 | CCUAGAUGUAAAAACUAGA |
| siRNA 4741 | 4741 | CUAGUUUUUACAUCUAGGA | 10231 | UCCUAGAUGUAAAAACUAG |
| siRNA 4742 | 4742 | UAGUUUUUACAUCUAGGAG | 10232 | CUCCUAGAUGUAAAAACUA |
| siRNA 4743 | 4743 | AGUUUUUACAUCUAGGAGA | 10233 | UCUCCUAGAUGUAAAAACU |
| siRNA 4744 | 4744 | GUUUUUACAUCUAGGAGAA | 10234 | UUCUCCUAGAUGUAAAAAC |
| siRNA 4745 | 4745 | UUUUUACAUCUAGGAGAAU | 10235 | AUUCUCCUAGAUGUAAAAA |
| siRNA 4746 | 4746 | UUUUACAUCUAGGAGAAUU | 10236 | AAUUCUCCUAGAUGUAAAA |
| siRNA 4747 | 4747 | UUUACAUCUAGGAGAAUUG | 10237 | CAAUUCUCCUAGAUGUAAA |
| siRNA 4748 | 4748 | UUACAUCUAGGAGAAUUGU | 10238 | ACAAUUCUCCUAGAUGUAA |
| siRNA 4749 | 4749 | UACAUCUAGGAGAAUUGUG | 10239 | CACAAUUCUCCUAGAUGUA |
| siRNA 4750 | 4750 | ACAUCUAGGAGAAUUGUGA | 10240 | UCACAAUUCUCCUAGAUGU |
| siRNA 4751 | 4751 | CAUCUAGGAGAAUUGUGAA | 10241 | UUCACAAUUCUCCUAGAUG |
| siRNA 4752 | 4752 | AUCUAGGAGAAUUGUGAAU | 10242 | AUUCACAAUUCUCCUAGAU |
| siRNA 4753 | 4753 | UCUAGGAGAAUUGUGAAUA | 10243 | UAUUCACAAUUCUCCUAGA |
| siRNA 4754 | 4754 | CUAGGAGAAUUGUGAAUAA | 10244 | UUAUUCACAAUUCUCCUAG |
| siRNA 4755 | 4755 | UAGGAGAAUUGUGAAUAAC | 10245 | GUUAUUCACAAUUCUCCUA |
| siRNA 4756 | 4756 | AGGAGAAUUGUGAAUAACA | 10246 | UGUUAUUCACAAUUCUCCU |
| siRNA 4757 | 4757 | GGAGAAUUGUGAAUAACAC | 10247 | GUGUUAUUCACAAUUCUCC |
| siRNA 4758 | 4758 | GAGAAUUGUGAAUAACACC | 10248 | GGUGUUAUUCACAAUUCUC |
| siRNA 4759 | 4759 | AGAAUUGUGAAUAACACCA | 10249 | UGGUGUUAUUCACAAUUCU |
| siRNA 4760 | 4760 | GAAUUGUGAAUAACACCAA | 10250 | UUGGUGUUAUUCACAAUUC |
| siRNA 4761 | 4761 | AAUUGUGAAUAACACCAAC | 10251 | GUUGGUGUUAUUCACAAUU |
| siRNA 4762 | 4762 | AUUGUGAAUAACACCAACU | 10252 | AGUUGGUGUUAUUCACAAU |
| siRNA 4763 | 4763 | UUGUGAAUAACACCAACUG | 10253 | CAGUUGGUGUUAUUCACAA |
| siRNA 4764 | 4764 | UGUGAAUAACACCAACUGU | 10254 | ACAGUUGGUGUUAUUCACA |
| siRNA 4765 | 4765 | GUGAAUAACACCAACUGUU | 10255 | AACAGUUGGUGUUAUUCAC |
| siRNA 4766 | 4766 | UGAAUAACACCAACUGUUC | 10256 | GAACAGUUGGUGUUAUUCA |
| siRNA 4767 | 4767 | GAAUAACACCAACUGUUCU | 10257 | AGAACAGUUGGUGUUAUUC |
| siRNA 4768 | 4768 | AAUAACACCAACUGUUCUU | 10258 | AAGAACAGUUGGUGUUAUU |
| siRNA 4769 | 4769 | AUAACACCAACUGUUCUUA | 10259 | UAAGAACAGUUGGUGUUAU |
| siRNA 4770 | 4770 | UAACACCAACUGUUCUUAA | 10260 | UUAAGAACAGUUGGUGUUA |
| siRNA 4771 | 4771 | AACACCAACUGUUCUUAAA | 10261 | UUUAAGAACAGUUGGUGUU |
| siRNA 4772 | 4772 | ACACCAACUGUUCUUAAAC | 10262 | GUUUAAGAACAGUUGGUGU |
| siRNA 4773 | 4773 | CACCAACUGUUCUUAAACU | 10263 | AGUUUAAGAACAGUUGGUG |
| siRNA 4774 | 4774 | ACCAACUGUUCUUAAACUC | 10264 | GAGUUUAAGAACAGUUGGU |
| siRNA 4775 | 4775 | CCAACUGUUCUUAAACUCU | 10265 | AGAGUUUAAGAACAGUUGG |
| siRNA 4776 | 4776 | CAACUGUUCUUAAACUCUU | 10266 | AAGAGUUUAAGAACAGUUG |
| siRNA 4777 | 4777 | AACUGUUCUUAAACUCUUU | 10267 | AAAGAGUUUAAGAACAGUU |
| siRNA 4778 | 4778 | ACUGUUCUUAAACUCUUUA | 10268 | UAAAGAGUUUAAGAACAGU |
| siRNA 4779 | 4779 | CUGUUCUUAAACUCUUUAA | 10269 | UUAAAGAGUUUAAGAACAG |
| siRNA 4780 | 4780 | UGUUCUUAAACUCUUUAAU | 10270 | AUUAAAGAGUUUAAGAACA |
| siRNA 4781 | 4781 | GUUCUUAAACUCUUUAAUG | 10271 | CAUUAAAGAGUUUAAGAAC |
| siRNA 4782 | 4782 | UUCUUAAACUCUUUAAUGC | 10272 | GCAUUAAAGAGUUUAAGAA |
| siRNA 4783 | 4783 | UCUUAAACUCUUUAAUGCC | 10273 | GGCAUUAAAGAGUUUAAGA |
| siRNA 4784 | 4784 | CUUAAACUCUUUAAUGCCA | 10274 | UGGCAUUAAAGAGUUUAAG |
| siRNA 4785 | 4785 | UUAAACUCUUUAAUGCCAU | 10275 | AUGGCAUUAAAGAGUUUAA |
| siRNA 4786 | 4786 | UAAACUCUUUAAUGCCAUG | 10276 | CAUGGCAUUAAAGAGUUUA |
| siRNA 4787 | 4787 | AAACUCUUUAAUGCCAUGU | 10277 | ACAUGGCAUUAAAGAGUUU |
| siRNA 4788 | 4788 | AACUCUUUAAUGCCAUGUC | 10278 | GACAUGGCAUUAAAGAGUU |
| siRNA 4789 | 4789 | ACUCUUUAAUGCCAUGUCU | 10279 | AGACAUGGCAUUAAAGAGU |
| siRNA 4790 | 4790 | CUCUUUAAUGCCAUGUCUU | 10280 | AAGACAUGGCAUUAAAGAG |
| siRNA 4791 | 4791 | UCUUUAAUGCCAUGUCUUA | 10281 | UAAGACAUGGCAUUAAAGA |
| siRNA 4792 | 4792 | CUUUAAUGCCAUGUCUUAA | 10282 | UUAAGACAUGGCAUUAAAG |
| siRNA 4793 | 4793 | UUUAAUGCCAUGUCUUAAA | 10283 | UUUAAGACAUGGCAUUAAA |
| siRNA 4794 | 4794 | UUAAUGCCAUGUCUUAAAU | 10284 | AUUUAAGACAUGGCAUUAA |
| siRNA 4795 | 4795 | UAAUGCCAUGUCUUAAAUG | 10285 | CAUUUAAGACAUGGCAUUA |
| siRNA 4796 | 4796 | AAUGCCAUGUCUUAAAUGC | 10286 | GCAUUUAAGACAUGGCAUU |
| siRNA 4797 | 4797 | AUGCCAUGUCUUAAAUGCC | 10287 | GGCAUUUAAGACAUGGCAU |
| siRNA 4798 | 4798 | UGCCAUGUCUUAAAUGCCG | 10288 | CGGCAUUUAAGACAUGGCA |
| siRNA 4799 | 4799 | GCCAUGUCUUAAAUGCCGG | 10289 | CCGGCAUUUAAGACAUGGC |
| siRNA 4800 | 4800 | CCAUGUCUUAAAUGCCGGU | 10290 | ACCGGCAUUUAAGACAUGG |
| siRNA 4801 | 4801 | CAUGUCUUAAAUGCCGGUA | 10291 | UACCGGCAUUUAAGACAUG |
| siRNA 4802 | 4802 | AUGUCUUAAAUGCCGGUAU | 10292 | AUACCGGCAUUUAAGACAU |
| siRNA 4803 | 4803 | UGUCUUAAAUGCCGGUAUU | 10293 | AAUACCGGCAUUUAAGACA |
| siRNA 4804 | 4804 | GUCUUAAAUGCCGGUAUUU | 10294 | AAAUACCGGCAUUUAAGAC |
| siRNA 4805 | 4805 | UCUUAAAUGCCGGUAUUUG | 10295 | CAAAUACCGGCAUUUAAGA |
| siRNA 4806 | 4806 | CUUAAAUGCCGGUAUUUGC | 10296 | GCAAAUACCGGCAUUUAAG |
| siRNA 4807 | 4807 | UUAAAUGCCGGUAUUUGCU | 10297 | AGCAAAUACCGGCAUUUAA |
| siRNA 4808 | 4808 | UAAAUGCCGGUAUUUGCUG | 10298 | CAGCAAAUACCGGCAUUUA |
| siRNA 4809 | 4809 | AAAUGCCGGUAUUUGCUGC | 10299 | GCAGCAAAUACCGGCAUUU |
| siRNA 4810 | 4810 | AAUGCCGGUAUUUGCUGCU | 10300 | AGCAGCAAAUACCGGCAUU |
| siRNA 4811 | 4811 | AUGCCGGUAUUUGCUGCUG | 10301 | CAGCAGCAAAUACCGGCAU |
| siRNA 4812 | 4812 | UGCCGGUAUUUGCUGCUGA | 10302 | UCAGCAGCAAAUACCGGCA |
| siRNA 4813 | 4813 | GCCGGUAUUUGCUGCUGAA | 10303 | UUCAGCAGCAAAUACCGGC |
| siRNA 4814 | 4814 | CCGGUAUUUGCUGCUGAAG | 10304 | CUUCAGCAGCAAAUACCGG |
| siRNA 4815 | 4815 | CGGUAUUUGCUGCUGAAGA | 10305 | UCUUCAGCAGCAAAUACCG |
| siRNA 4816 | 4816 | GGUAUUUGCUGCUGAAGAC | 10306 | GUCUUCAGCAGCAAAUACC |
| siRNA 4817 | 4817 | GUAUUUGCUGCUGAAGACA | 10307 | UGUCUUCAGCAGCAAAUAC |
| siRNA 4818 | 4818 | UAUUUGCUGCUGAAGACAA | 10308 | UUGUCUUCAGCAGCAAAUA |
| siRNA 4819 | 4819 | AUUUGCUGCUGAAGACAAA | 10309 | UUUGUCUUCAGCAGCAAAU |
| siRNA 4820 | 4820 | UUUGCUGCUGAAGACAAAA | 10310 | UUUUGUCUUCAGCAGCAAA |
| siRNA 4821 | 4821 | UUGCUGCUGAAGACAAAAA | 10311 | UUUUUGUCUUCAGCAGCAA |
| siRNA 4822 | 4822 | UGCUGCUGAAGACAAAAAU | 10312 | AUUUUUGUCUUCAGCAGCA |
| siRNA 4823 | 4823 | GCUGCUGAAGACAAAAAUG | 10313 | CAUUUUUGUCUUCAGCAGC |
| siRNA 4824 | 4824 | CUGCUGAAGACAAAAAUGA | 10314 | UCAUUUUUGUCUUCAGCAG |
| siRNA 4825 | 4825 | UGCUGAAGACAAAAAUGAA | 10315 | UUCAUUUUUGUCUUCAGCA |
| siRNA 4826 | 4826 | GCUGAAGACAAAAAUGAAA | 10316 | UUUCAUUUUUGUCUUCAGC |
| siRNA 4827 | 4827 | CUGAAGACAAAAAUGAAAA | 10317 | UUUUCAUUUUUGUCUUCAG |
| siRNA 4828 | 4828 | UGAAGACAAAAAUGAAAAG | 10318 | CUUUUCAUUUUUGUCUUCA |
| siRNA 4829 | 4829 | GAAGACAAAAAUGAAAAGU | 10319 | ACUUUUCAUUUUUGUCUUC |
| siRNA 4830 | 4830 | AAGACAAAAAUGAAAAGUA | 10320 | UACUUUUCAUUUUUGUCUU |
| siRNA 4831 | 4831 | AGACAAAAAUGAAAAGUAG | 10321 | CUACUUUUCAUUUUUGUCU |
| siRNA 4832 | 4832 | GACAAAAAUGAAAAGUAGG | 10322 | CCUACUUUUCAUUUUUGUC |
| siRNA 4833 | 4833 | ACAAAAAUGAAAAGUAGGA | 10323 | UCCUACUUUUCAUUUUUGU |
| siRNA 4834 | 4834 | CAAAAAUGAAAAGUAGGAU | 10324 | AUCCUACUUUUCAUUUUUG |
| siRNA 4835 | 4835 | AAAAAUGAAAAGUAGGAUG | 10325 | CAUCCUACUUUUCAUUUUU |
| siRNA 4836 | 4836 | AAAAUGAAAAGUAGGAUGA | 10326 | UCAUCCUACUUUUCAUUUU |
| siRNA 4837 | 4837 | AAAUGAAAAGUAGGAUGAA | 10327 | UUCAUCCUACUUUUCAUUU |
| siRNA 4838 | 4838 | AAUGAAAAGUAGGAUGAAA | 10328 | UUUCAUCCUACUUUUCAUU |
| siRNA 4839 | 4839 | AUGAAAAGUAGGAUGAAAA | 10329 | UUUUCAUCCUACUUUUCAU |
| siRNA 4840 | 4840 | UGAAAAGUAGGAUGAAAAU | 10330 | AUUUUCAUCCUACUUUUCA |
| siRNA 4841 | 4841 | GAAAAGUAGGAUGAAAAUA | 10331 | UAUUUUCAUCCUACUUUUC |
| siRNA 4842 | 4842 | AAAAGUAGGAUGAAAAUAA | 10332 | UUAUUUUCAUCCUACUUUU |
| siRNA 4843 | 4843 | AAAGUAGGAUGAAAAUAAU | 10333 | AUUAUUUUCAUCCUACUUU |
| siRNA 4844 | 4844 | AAGUAGGAUGAAAAUAAUA | 10334 | UAUUAUUUUCAUCCUACUU |
| siRNA 4845 | 4845 | AGUAGGAUGAAAAUAAUAG | 10335 | CUAUUAUUUUCAUCCUACU |
| siRNA 4846 | 4846 | GUAGGAUGAAAAUAAUAGA | 10336 | UCUAUUAUUUUCAUCCUAC |
| siRNA 4847 | 4847 | UAGGAUGAAAAUAAUAGAA | 10337 | UUCUAUUAUUUUCAUCCUA |
| siRNA 4848 | 4848 | AGGAUGAAAAUAAUAGAAU | 10338 | AUUCUAUUAUUUUCAUCCU |
| siRNA 4849 | 4849 | GGAUGAAAAUAAUAGAAUG | 10339 | CAUUCUAUUAUUUUCAUCC |
| siRNA 4850 | 4850 | GAUGAAAAUAAUAGAAUGG | 10340 | CCAUUCUAUUAUUUUCAUC |
| siRNA 4851 | 4851 | AUGAAAAUAAUAGAAUGGC | 10341 | GCCAUUCUAUUAUUUUCAU |
| siRNA 4852 | 4852 | UGAAAAUAAUAGAAUGGCA | 10342 | UGCCAUUCUAUUAUUUUCA |
| siRNA 4853 | 4853 | GAAAAUAAUAGAAUGGCAC | 10343 | GUGCCAUUCUAUUAUUUUC |
| siRNA 4854 | 4854 | AAAAUAAUAGAAUGGCACU | 10344 | AGUGCCAUUCUAUUAUUUU |
| siRNA 4855 | 4855 | AAAUAAUAGAAUGGCACUG | 10345 | CAGUGCCAUUCUAUUAUUU |
| siRNA 4856 | 4856 | AAUAAUAGAAUGGCACUGU | 10346 | ACAGUGCCAUUCUAUUAUU |
| siRNA 4857 | 4857 | AUAAUAGAAUGGCACUGUA | 10347 | UACAGUGCCAUUCUAUUAU |
| siRNA 4858 | 4858 | UAAUAGAAUGGCACUGUAA | 10348 | UUACAGUGCCAUUCUAUUA |
| siRNA 4859 | 4859 | AAUAGAAUGGCACUGUAAG | 10349 | CUUACAGUGCCAUUCUAUU |
| siRNA 4860 | 4860 | AUAGAAUGGCACUGUAAGU | 10350 | ACUUACAGUGCCAUUCUAU |
| siRNA 4861 | 4861 | UAGAAUGGCACUGUAAGUG | 10351 | CACUUACAGUGCCAUUCUA |
| siRNA 4862 | 4862 | AGAAUGGCACUGUAAGUGU | 10352 | ACACUUACAGUGCCAUUCU |
| siRNA 4863 | 4863 | GAAUGGCACUGUAAGUGUU | 10353 | AACACUUACAGUGCCAUUC |
| siRNA 4864 | 4864 | AAUGGCACUGUAAGUGUUU | 10354 | AAACACUUACAGUGCCAUU |
| siRNA 4865 | 4865 | AUGGCACUGUAAGUGUUUA | 10355 | UAAACACUUACAGUGCCAU |
| siRNA 4866 | 4866 | UGGCACUGUAAGUGUUUAU | 10356 | AUAAACACUUACAGUGCCA |
| siRNA 4867 | 4867 | GGCACUGUAAGUGUUUAUU | 10357 | AAUAAACACUUACAGUGCC |
| siRNA 4868 | 4868 | GCACUGUAAGUGUUUAUUA | 10358 | UAAUAAACACUUACAGUGC |
| siRNA 4869 | 4869 | CACUGUAAGUGUUUAUUAU | 10359 | AUAAUAAACACUUACAGUG |
| siRNA 4870 | 4870 | ACUGUAAGUGUUUAUUAUU | 10360 | AAUAAUAAACACUUACAGU |
| siRNA 4871 | 4871 | CUGUAAGUGUUUAUUAUUU | 10361 | AAAUAAUAAACACUUACAG |
| siRNA 4872 | 4872 | UGUAAGUGUUUAUUAUUUU | 10362 | AAAAUAAUAAACACUUACA |
| siRNA 4873 | 4873 | GUAAGUGUUUAUUAUUUUG | 10363 | CAAAAUAAUAAACACUUAC |
| siRNA 4874 | 4874 | UAAGUGUUUAUUAUUUUGU | 10364 | ACAAAAUAAUAAACACUUA |
| siRNA 4875 | 4875 | AAGUGUUUAUUAUUUUGUC | 10365 | GACAAAAUAAUAAACACUU |
| siRNA 4876 | 4876 | AGUGUUUAUUAUUUUGUCA | 10366 | UGACAAAAUAAUAAACACU |
| siRNA 4877 | 4877 | GUGUUUAUUAUUUUGUCAA | 10367 | UUGACAAAAUAAUAAACAC |
| siRNA 4878 | 4878 | UGUUUAUUAUUUUGUCAAA | 10368 | UUUGACAAAAUAAUAAACA |
| siRNA 4879 | 4879 | GUUUAUUAUUUUGUCAAAA | 10369 | UUUUGACAAAAUAAUAAAC |
| siRNA 4880 | 4880 | UUUAUUAUUUUGUCAAAAU | 10370 | AUUUUGACAAAAUAAUAAA |
| siRNA 4881 | 4881 | UUAUUAUUUUGUCAAAAUG | 10371 | CAUUUUGACAAAAUAAUAA |
| siRNA 4882 | 4882 | UAUUAUUUUGUCAAAAUGU | 10372 | ACAUUUUGACAAAAUAAUA |
| siRNA 4883 | 4883 | AUUAUUUUGUCAAAAUGUA | 10373 | UACAUUUUGACAAAAUAAU |
| siRNA 4884 | 4884 | UUAUUUUGUCAAAAUGUAA | 10374 | UUACAUUUUGACAAAAUAA |
| siRNA 4885 | 4885 | UAUUUUGUCAAAAUGUAAA | 10375 | UUUACAUUUUGACAAAAUA |
| siRNA 4886 | 4886 | AUUUUGUCAAAAUGUAAAC | 10376 | GUUUACAUUUUGACAAAAU |
| siRNA 4887 | 4887 | UUUUGUCAAAAUGUAAACA | 10377 | UGUUUACAUUUUGACAAAA |
| siRNA 4888 | 4888 | UUUGUCAAAAUGUAAACAA | 10378 | UUGUUUACAUUUUGACAAA |
| siRNA 4889 | 4889 | UUGUCAAAAUGUAAACAAA | 10379 | UUUGUUUACAUUUUGACAA |
| siRNA 4890 | 4890 | UGUCAAAAUGUAAACAAAG | 10380 | CUUUGUUUACAUUUUGACA |
| siRNA 4891 | 4891 | GUCAAAAUGUAAACAAAGA | 10381 | UCUUUGUUUACAUUUUGAC |
| siRNA 4892 | 4892 | UCAAAAUGUAAACAAAGAC | 10382 | GUCUUUGUUUACAUUUUGA |
| siRNA 4893 | 4893 | CAAAAUGUAAACAAAGACU | 10383 | AGUCUUUGUUUACAUUUUG |
| siRNA 4894 | 4894 | AAAAUGUAAACAAAGACUA | 10384 | UAGUCUUUGUUUACAUUUU |
| siRNA 4895 | 4895 | AAAUGUAAACAAAGACUAC | 10385 | GUAGUCUUUGUUUACAUUU |
| siRNA 4896 | 4896 | AAUGUAAACAAAGACUACA | 10386 | UGUAGUCUUUGUUUACAUU |
| siRNA 4897 | 4897 | AUGUAAACAAAGACUACAU | 10387 | AUGUAGUCUUUGUUUACAU |
| siRNA 4898 | 4898 | UGUAAACAAAGACUACAUA | 10388 | UAUGUAGUCUUUGUUUACA |
| siRNA 4899 | 4899 | GUAAACAAAGACUACAUAA | 10389 | UUAUGUAGUCUUUGUUUAC |
| siRNA 4900 | 4900 | UAAACAAAGACUACAUAAC | 10390 | GUUAUGUAGUCUUUGUUUA |
| siRNA 4901 | 4901 | AAACAAAGACUACAUAACC | 10391 | GGUUAUGUAGUCUUUGUUU |
| siRNA 4902 | 4902 | AACAAAGACUACAUAACCC | 10392 | GGGUUAUGUAGUCUUUGUU |
| siRNA 4903 | 4903 | ACAAAGACUACAUAACCCA | 10393 | UGGGUUAUGUAGUCUUUGU |
| siRNA 4904 | 4904 | CAAAGACUACAUAACCCAA | 10394 | UUGGGUUAUGUAGUCUUUG |
| siRNA 4905 | 4905 | AAAGACUACAUAACCCAAU | 10395 | AUUGGGUUAUGUAGUCUUU |
| siRNA 4906 | 4906 | AAGACUACAUAACCCAAUG | 10396 | CAUUGGGUUAUGUAGUCUU |
| siRNA 4907 | 4907 | AGACUACAUAACCCAAUGA | 10397 | UCAUUGGGUUAUGUAGUCU |
| siRNA 4908 | 4908 | GACUACAUAACCCAAUGAU | 10398 | AUCAUUGGGUUAUGUAGUC |
| siRNA 4909 | 4909 | ACUACAUAACCCAAUGAUG | 10399 | CAUCAUUGGGUUAUGUAGU |
| siRNA 4910 | 4910 | CUACAUAACCCAAUGAUGG | 10400 | CCAUCAUUGGGUUAUGUAG |
| siRNA 4911 | 4911 | UACAUAACCCAAUGAUGGA | 10401 | UCCAUCAUUGGGUUAUGUA |
| siRNA 4912 | 4912 | ACAUAACCCAAUGAUGGAG | 10402 | CUCCAUCAUUGGGUUAUGU |
| siRNA 4913 | 4913 | CAUAACCCAAUGAUGGAGG | 10403 | CCUCCAUCAUUGGGUUAUG |
| siRNA 4914 | 4914 | AUAACCCAAUGAUGGAGGG | 10404 | CCCUCCAUCAUUGGGUUAU |
| siRNA 4915 | 4915 | UAACCCAAUGAUGGAGGGA | 10405 | UCCCUCCAUCAUUGGGUUA |
| siRNA 4916 | 4916 | AACCCAAUGAUGGAGGGAA | 10406 | UUCCCUCCAUCAUUGGGUU |
| siRNA 4917 | 4917 | ACCCAAUGAUGGAGGGAAA | 10407 | UUUCCCUCCAUCAUUGGGU |
| siRNA 4918 | 4918 | CCCAAUGAUGGAGGGAAAA | 10408 | UUUUCCCUCCAUCAUUGGG |
| siRNA 4919 | 4919 | CCAAUGAUGGAGGGAAAAA | 10409 | UUUUUCCCUCCAUCAUUGG |
| siRNA 4920 | 4920 | CAAUGAUGGAGGGAAAAAG | 10410 | CUUUUUCCCUCCAUCAUUG |
| siRNA 4921 | 4921 | AAUGAUGGAGGGAAAAAGG | 10411 | CCUUUUUCCCUCCAUCAUU |
| siRNA 4922 | 4922 | AUGAUGGAGGGAAAAAGGG | 10412 | CCCUUUUUCCCUCCAUCAU |
| siRNA 4923 | 4923 | UGAUGGAGGGAAAAAGGGC | 10413 | GCCCUUUUUCCCUCCAUCA |
| siRNA 4924 | 4924 | GAUGGAGGGAAAAAGGGCA | 10414 | UGCCCUUUUUCCCUCCAUC |
| siRNA 4925 | 4925 | AUGGAGGGAAAAAGGGCAU | 10415 | AUGCCCUUUUUCCCUCCAU |
| siRNA 4926 | 4926 | UGGAGGGAAAAAGGGCAUG | 10416 | CAUGCCCUUUUUCCCUCCA |
| siRNA 4927 | 4927 | GGAGGGAAAAAGGGCAUGU | 10417 | ACAUGCCCUUUUUCCCUCC |
| siRNA 4928 | 4928 | GAGGGAAAAAGGGCAUGUA | 10418 | UACAUGCCCUUUUUCCCUC |
| siRNA 4929 | 4929 | AGGGAAAAAGGGCAUGUAU | 10419 | AUACAUGCCCUUUUUCCCU |
| siRNA 4930 | 4930 | GGGAAAAAGGGCAUGUAUC | 10420 | GAUACAUGCCCUUUUUCCC |
| siRNA 4931 | 4931 | GGAAAAAGGGCAUGUAUCU | 10421 | AGAUACAUGCCCUUUUUCC |
| siRNA 4932 | 4932 | GAAAAAGGGCAUGUAUCUC | 10422 | GAGAUACAUGCCCUUUUUC |
| siRNA 4933 | 4933 | AAAAAGGGCAUGUAUCUCA | 10423 | UGAGAUACAUGCCCUUUUU |
| siRNA 4934 | 4934 | AAAAGGGCAUGUAUCUCAU | 10424 | AUGAGAUACAUGCCCUUUU |
| siRNA 4935 | 4935 | AAAGGGCAUGUAUCUCAUU | 10425 | AAUGAGAUACAUGCCCUUU |
| siRNA 4936 | 4936 | AAGGGCAUGUAUCUCAUUC | 10426 | GAAUGAGAUACAUGCCCUU |
| siRNA 4937 | 4937 | AGGGCAUGUAUCUCAUUCA | 10427 | UGAAUGAGAUACAUGCCCU |
| siRNA 4938 | 4938 | GGGCAUGUAUCUCAUUCAG | 10428 | CUGAAUGAGAUACAUGCCC |
| siRNA 4939 | 4939 | GGCAUGUAUCUCAUUCAGA | 10429 | UCUGAAUGAGAUACAUGCC |
| siRNA 4940 | 4940 | GCAUGUAUCUCAUUCAGAU | 10430 | AUCUGAAUGAGAUACAUGC |
| siRNA 4941 | 4941 | CAUGUAUCUCAUUCAGAUG | 10431 | CAUCUGAAUGAGAUACAUG |
| siRNA 4942 | 4942 | AUGUAUCUCAUUCAGAUGU | 10432 | ACAUCUGAAUGAGAUACAU |
| siRNA 4943 | 4943 | UGUAUCUCAUUCAGAUGUG | 10433 | CACAUCUGAAUGAGAUACA |
| siRNA 4944 | 4944 | GUAUCUCAUUCAGAUGUGC | 10434 | GCACAUCUGAAUGAGAUAC |
| siRNA 4945 | 4945 | UAUCUCAUUCAGAUGUGCC | 10435 | GGCACAUCUGAAUGAGAUA |
| siRNA 4946 | 4946 | AUCUCAUUCAGAUGUGCCU | 10436 | AGGCACAUCUGAAUGAGAU |
| siRNA 4947 | 4947 | UCUCAUUCAGAUGUGCCUU | 10437 | AAGGCACAUCUGAAUGAGA |
| siRNA 4948 | 4948 | CUCAUUCAGAUGUGCCUUU | 10438 | AAAGGCACAUCUGAAUGAG |
| siRNA 4949 | 4949 | UCAUUCAGAUGUGCCUUUU | 10439 | AAAAGGCACAUCUGAAUGA |
| siRNA 4950 | 4950 | CAUUCAGAUGUGCCUUUUG | 10440 | CAAAAGGCACAUCUGAAUG |
| siRNA 4951 | 4951 | AUUCAGAUGUGCCUUUUGU | 10441 | ACAAAAGGCACAUCUGAAU |
| siRNA 4952 | 4952 | UUCAGAUGUGCCUUUUGUU | 10442 | AACAAAAGGCACAUCUGAA |
| siRNA 4953 | 4953 | UCAGAUGUGCCUUUUGUUU | 10443 | AAACAAAAGGCACAUCUGA |
| siRNA 4954 | 4954 | CAGAUGUGCCUUUUGUUUU | 10444 | AAAACAAAAGGCACAUCUG |
| siRNA 4955 | 4955 | AGAUGUGCCUUUUGUUUUU | 10445 | AAAAACAAAAGGCACAUCU |
| siRNA 4956 | 4956 | GAUGUGCCUUUUGUUUUUG | 10446 | CAAAAACAAAAGGCACAUC |
| siRNA 4957 | 4957 | AUGUGCCUUUUGUUUUUGC | 10447 | GCAAAAACAAAAGGCACAU |
| siRNA 4958 | 4958 | UGUGCCUUUUGUUUUUGCA | 10448 | UGCAAAAACAAAAGGCACA |
| siRNA 4959 | 4959 | GUGCCUUUUGUUUUUGCAG | 10449 | CUGCAAAAACAAAAGGCAC |
| siRNA 4960 | 4960 | UGCCUUUUGUUUUUGCAGA | 10450 | UCUGCAAAAACAAAAGGCA |
| siRNA 4961 | 4961 | GCCUUUUGUUUUUGCAGAC | 10451 | GUCUGCAAAAACAAAAGGC |
| siRNA 4962 | 4962 | CCUUUUGUUUUUGCAGACU | 10452 | AGUCUGCAAAAACAAAAGG |
| siRNA 4963 | 4963 | CUUUUGUUUUUGCAGACUA | 10453 | UAGUCUGCAAAAACAAAAG |
| siRNA 4964 | 4964 | UUUUGUUUUUGCAGACUAU | 10454 | AUAGUCUGCAAAAACAAAA |
| siRNA 4965 | 4965 | UUUGUUUUUGCAGACUAUG | 10455 | CAUAGUCUGCAAAAACAAA |
| siRNA 4966 | 4966 | UUGUUUUUGCAGACUAUGA | 10456 | UCAUAGUCUGCAAAAACAA |
| siRNA 4967 | 4967 | UGUUUUUGCAGACUAUGAC | 10457 | GUCAUAGUCUGCAAAAACA |
| siRNA 4968 | 4968 | GUUUUUGCAGACUAUGACG | 10458 | CGUCAUAGUCUGCAAAAAC |
| siRNA 4969 | 4969 | UUUUUGCAGACUAUGACGU | 10459 | ACGUCAUAGUCUGCAAAAA |
| siRNA 4970 | 4970 | UUUUGCAGACUAUGACGUC | 10460 | GACGUCAUAGUCUGCAAAA |
| siRNA 4971 | 4971 | UUUGCAGACUAUGACGUCU | 10461 | AGACGUCAUAGUCUGCAAA |
| siRNA 4972 | 4972 | UUGCAGACUAUGACGUCUU | 10462 | AAGACGUCAUAGUCUGCAA |
| siRNA 4973 | 4973 | UGCAGACUAUGACGUCUUU | 10463 | AAAGACGUCAUAGUCUGCA |
| siRNA 4974 | 4974 | GCAGACUAUGACGUCUUUA | 10464 | UAAAGACGUCAUAGUCUGC |
| siRNA 4975 | 4975 | CAGACUAUGACGUCUUUAG | 10465 | CUAAAGACGUCAUAGUCUG |
| siRNA 4976 | 4976 | AGACUAUGACGUCUUUAGC | 10466 | GCUAAAGACGUCAUAGUCU |
| siRNA 4977 | 4977 | GACUAUGACGUCUUUAGCU | 10467 | AGCUAAAGACGUCAUAGUC |
| siRNA 4978 | 4978 | ACUAUGACGUCUUUAGCUA | 10468 | UAGCUAAAGACGUCAUAGU |
| siRNA 4979 | 4979 | CUAUGACGUCUUUAGCUAA | 10469 | UUAGCUAAAGACGUCAUAG |
| siRNA 4980 | 4980 | UAUGACGUCUUUAGCUAAU | 10470 | AUUAGCUAAAGACGUCAUA |
| siRNA 4981 | 4981 | AUGACGUCUUUAGCUAAUG | 10471 | CAUUAGCUAAAGACGUCAU |
| siRNA 4982 | 4982 | UGACGUCUUUAGCUAAUGA | 10472 | UCAUUAGCUAAAGACGUCA |
| siRNA 4983 | 4983 | GACGUCUUUAGCUAAUGAA | 10473 | UUCAUUAGCUAAAGACGUC |
| siRNA 4984 | 4984 | ACGUCUUUAGCUAAUGAAU | 10474 | AUUCAUUAGCUAAAGACGU |
| siRNA 4985 | 4985 | CGUCUUUAGCUAAUGAAUU | 10475 | AAUUCAUUAGCUAAAGACG |
| siRNA 4986 | 4986 | GUCUUUAGCUAAUGAAUUG | 10476 | CAAUUCAUUAGCUAAAGAC |
| siRNA 4987 | 4987 | UCUUUAGCUAAUGAAUUGC | 10477 | GCAAUUCAUUAGCUAAAGA |
| siRNA 4988 | 4988 | CUUUAGCUAAUGAAUUGCC | 10478 | GGCAAUUCAUUAGCUAAAG |
| siRNA 4989 | 4989 | UUUAGCUAAUGAAUUGCCU | 10479 | AGGCAAUUCAUUAGCUAAA |
| siRNA 4990 | 4990 | UUAGCUAAUGAAUUGCCUA | 10480 | UAGGCAAUUCAUUAGCUAA |
| siRNA 4991 | 4991 | UAGCUAAUGAAUUGCCUAU | 10481 | AUAGGCAAUUCAUUAGCUA |
| siRNA 4992 | 4992 | AGCUAAUGAAUUGCCUAUU | 10482 | AAUAGGCAAUUCAUUAGCU |
| siRNA 4993 | 4993 | GCUAAUGAAUUGCCUAUUG | 10483 | CAAUAGGCAAUUCAUUAGC |
| siRNA 4994 | 4994 | CUAAUGAAUUGCCUAUUGU | 10484 | ACAAUAGGCAAUUCAUUAG |
| siRNA 4995 | 4995 | UAAUGAAUUGCCUAUUGUU | 10485 | AACAAUAGGCAAUUCAUUA |
| siRNA 4996 | 4996 | AAUGAAUUGCCUAUUGUUA | 10486 | UAACAAUAGGCAAUUCAUU |
| siRNA 4997 | 4997 | AUGAAUUGCCUAUUGUUAU | 10487 | AUAACAAUAGGCAAUUCAU |
| siRNA 4998 | 4998 | UGAAUUGCCUAUUGUUAUG | 10488 | CAUAACAAUAGGCAAUUCA |
| siRNA 4999 | 4999 | GAAUUGCCUAUUGUUAUGG | 10489 | CCAUAACAAUAGGCAAUUC |
| siRNA 5000 | 5000 | AAUUGCCUAUUGUUAUGGA | 10490 | UCCAUAACAAUAGGCAAUU |
| siRNA 5001 | 5001 | AUUGCCUAUUGUUAUGGAA | 10491 | UUCCAUAACAAUAGGCAAU |
| siRNA 5002 | 5002 | UUGCCUAUUGUUAUGGAAA | 10492 | UUUCCAUAACAAUAGGCAA |
| siRNA 5003 | 5003 | UGCCUAUUGUUAUGGAAAA | 10493 | UUUUCCAUAACAAUAGGCA |
| siRNA 5004 | 5004 | GCCUAUUGUUAUGGAAAAC | 10494 | GUUUUCCAUAACAAUAGGC |
| siRNA 5005 | 5005 | CCUAUUGUUAUGGAAAACA | 10495 | UGUUUUCCAUAACAAUAGG |
| siRNA 5006 | 5006 | CUAUUGUUAUGGAAAACAG | 10496 | CUGUUUUCCAUAACAAUAG |
| siRNA 5007 | 5007 | UAUUGUUAUGGAAAACAGU | 10497 | ACUGUUUUCCAUAACAAUA |
| siRNA 5008 | 5008 | AUUGUUAUGGAAAACAGUU | 10498 | AACUGUUUUCCAUAACAAU |
| siRNA 5009 | 5009 | UUGUUAUGGAAAACAGUUA | 10499 | UAACUGUUUUCCAUAACAA |
| siRNA 5010 | 5010 | UGUUAUGGAAAACAGUUAA | 10500 | UUAACUGUUUUCCAUAACA |
| siRNA 5011 | 5011 | GUUAUGGAAAACAGUUAAU | 10501 | AUUAACUGUUUUCCAUAAC |
| siRNA 5012 | 5012 | UUAUGGAAAACAGUUAAUA | 10502 | UAUUAACUGUUUUCCAUAA |
| siRNA 5013 | 5013 | UAUGGAAAACAGUUAAUAU | 10503 | AUAUUAACUGUUUUCCAUA |
| siRNA 5014 | 5014 | AUGGAAAACAGUUAAUAUG | 10504 | CAUAUUAACUGUUUUCCAU |
| siRNA 5015 | 5015 | UGGAAAACAGUUAAUAUGC | 10505 | GCAUAUUAACUGUUUUCCA |
| siRNA 5016 | 5016 | GGAAAACAGUUAAUAUGCC | 10506 | GGCAUAUUAACUGUUUUCC |
| siRNA 5017 | 5017 | GAAAACAGUUAAUAUGCCA | 10507 | UGGCAUAUUAACUGUUUUC |
| siRNA 5018 | 5018 | AAAACAGUUAAUAUGCCAU | 10508 | AUGGCAUAUUAACUGUUUU |
| siRNA 5019 | 5019 | AAACAGUUAAUAUGCCAUG | 10509 | CAUGGCAUAUUAACUGUUU |
| siRNA 5020 | 5020 | AACAGUUAAUAUGCCAUGU | 10510 | ACAUGGCAUAUUAACUGUU |
| siRNA 5021 | 5021 | ACAGUUAAUAUGCCAUGUA | 10511 | UACAUGGCAUAUUAACUGU |
| siRNA 5022 | 5022 | CAGUUAAUAUGCCAUGUAU | 10512 | AUACAUGGCAUAUUAACUG |
| siRNA 5023 | 5023 | AGUUAAUAUGCCAUGUAUG | 10513 | CAUACAUGGCAUAUUAACU |
| siRNA 5024 | 5024 | GUUAAUAUGCCAUGUAUGU | 10514 | ACAUACAUGGCAUAUUAAC |
| siRNA 5025 | 5025 | UUAAUAUGCCAUGUAUGUA | 10515 | UACAUACAUGGCAUAUUAA |
| siRNA 5026 | 5026 | UAAUAUGCCAUGUAUGUAC | 10516 | GUACAUACAUGGCAUAUUA |
| siRNA 5027 | 5027 | AAUAUGCCAUGUAUGUACA | 10517 | UGUACAUACAUGGCAUAUU |
| siRNA 5028 | 5028 | AUAUGCCAUGUAUGUACAG | 10518 | CUGUACAUACAUGGCAUAU |
| siRNA 5029 | 5029 | UAUGCCAUGUAUGUACAGU | 10519 | ACUGUACAUACAUGGCAUA |
| siRNA 5030 | 5030 | AUGCCAUGUAUGUACAGUU | 10520 | AACUGUACAUACAUGGCAU |
| siRNA 5031 | 5031 | UGCCAUGUAUGUACAGUUU | 10521 | AAACUGUACAUACAUGGCA |
| siRNA 5032 | 5032 | GCCAUGUAUGUACAGUUUU | 10522 | AAAACUGUACAUACAUGGC |
| siRNA 5033 | 5033 | CCAUGUAUGUACAGUUUUG | 10523 | CAAAACUGUACAUACAUGG |
| siRNA 5034 | 5034 | CAUGUAUGUACAGUUUUGU | 10524 | ACAAAACUGUACAUACAUG |
| siRNA 5035 | 5035 | AUGUAUGUACAGUUUUGUU | 10525 | AACAAAACUGUACAUACAU |
| siRNA 5036 | 5036 | UGUAUGUACAGUUUUGUUU | 10526 | AAACAAAACUGUACAUACA |
| siRNA 5037 | 5037 | GUAUGUACAGUUUUGUUUA | 10527 | UAAACAAAACUGUACAUAC |
| siRNA 5038 | 5038 | UAUGUACAGUUUUGUUUAU | 10528 | AUAAACAAAACUGUACAUA |
| siRNA 5039 | 5039 | AUGUACAGUUUUGUUUAUA | 10529 | UAUAAACAAAACUGUACAU |
| siRNA 5040 | 5040 | UGUACAGUUUUGUUUAUAU | 10530 | AUAUAAACAAAACUGUACA |
| siRNA 5041 | 5041 | GUACAGUUUUGUUUAUAUU | 10531 | AAUAUAAACAAAACUGUAC |
| siRNA 5042 | 5042 | UACAGUUUUGUUUAUAUUG | 10532 | CAAUAUAAACAAAACUGUA |
| siRNA 5043 | 5043 | ACAGUUUUGUUUAUAUUGU | 10533 | ACAAUAUAAACAAAACUGU |
| siRNA 5044 | 5044 | CAGUUUUGUUUAUAUUGUA | 10534 | UACAAUAUAAACAAAACUG |
| siRNA 5045 | 5045 | AGUUUUGUUUAUAUUGUAU | 10535 | AUACAAUAUAAACAAAACU |
| siRNA 5046 | 5046 | GUUUUGUUUAUAUUGUAUA | 10536 | UAUACAAUAUAAACAAAAC |
| siRNA 5047 | 5047 | UUUUGUUUAUAUUGUAUAU | 10537 | AUAUACAAUAUAAACAAAA |
| siRNA 5048 | 5048 | UUUGUUUAUAUUGUAUAUU | 10538 | AAUAUACAAUAUAAACAAA |
| siRNA 5049 | 5049 | UUGUUUAUAUUGUAUAUUU | 10539 | AAAUAUACAAUAUAAACAA |
| siRNA 5050 | 5050 | UGUUUAUAUUGUAUAUUUA | 10540 | UAAAUAUACAAUAUAAACA |
| siRNA 5051 | 5051 | GUUUAUAUUGUAUAUUUAA | 10541 | UUAAAUAUACAAUAUAAAC |
| siRNA 5052 | 5052 | UUUAUAUUGUAUAUUUAAA | 10542 | UUUAAAUAUACAAUAUAAA |
| siRNA 5053 | 5053 | UUAUAUUGUAUAUUUAAAG | 10543 | CUUUAAAUAUACAAUAUAA |
| siRNA 5054 | 5054 | UAUAUUGUAUAUUUAAAGA | 10544 | UCUUUAAAUAUACAAUAUA |
| siRNA 5055 | 5055 | AUAUUGUAUAUUUAAAGAU | 10545 | AUCUUUAAAUAUACAAUAU |
| siRNA 5056 | 5056 | UAUUGUAUAUUUAAAGAUA | 10546 | UAUCUUUAAAUAUACAAUA |
| siRNA 5057 | 5057 | AUUGUAUAUUUAAAGAUAC | 10547 | GUAUCUUUAAAUAUACAAU |
| siRNA 5058 | 5058 | UUGUAUAUUUAAAGAUACU | 10548 | AGUAUCUUUAAAUAUACAA |
| siRNA 5059 | 5059 | UGUAUAUUUAAAGAUACUG | 10549 | CAGUAUCUUUAAAUAUACA |
| siRNA 5060 | 5060 | GUAUAUUUAAAGAUACUGC | 10550 | GCAGUAUCUUUAAAUAUAC |
| siRNA 5061 | 5061 | UAUAUUUAAAGAUACUGCU | 10551 | AGCAGUAUCUUUAAAUAUA |
| siRNA 5062 | 5062 | AUAUUUAAAGAUACUGCUA | 10552 | UAGCAGUAUCUUUAAAUAU |
| siRNA 5063 | 5063 | UAUUUAAAGAUACUGCUAA | 10553 | UUAGCAGUAUCUUUAAAUA |
| siRNA 5064 | 5064 | AUUUAAAGAUACUGCUAAU | 10554 | AUUAGCAGUAUCUUUAAAU |
| siRNA 5065 | 5065 | UUUAAAGAUACUGCUAAUA | 10555 | UAUUAGCAGUAUCUUUAAA |
| siRNA 5066 | 5066 | UUAAAGAUACUGCUAAUAA | 10556 | UUAUUAGCAGUAUCUUUAA |
| siRNA 5067 | 5067 | UAAAGAUACUGCUAAUAAC | 10557 | GUUAUUAGCAGUAUCUUUA |
| siRNA 5068 | 5068 | AAAGAUACUGCUAAUAACC | 10558 | GGUUAUUAGCAGUAUCUUU |
| siRNA 5069 | 5069 | AAGAUACUGCUAAUAACCU | 10559 | AGGUUAUUAGCAGUAUCUU |
| siRNA 5070 | 5070 | AGAUACUGCUAAUAACCUA | 10560 | UAGGUUAUUAGCAGUAUCU |
| siRNA 5071 | 5071 | GAUACUGCUAAUAACCUAU | 10561 | AUAGGUUAUUAGCAGUAUC |
| siRNA 5072 | 5072 | AUACUGCUAAUAACCUAUA | 10562 | UAUAGGUUAUUAGCAGUAU |
| siRNA 5073 | 5073 | UACUGCUAAUAACCUAUAU | 10563 | AUAUAGGUUAUUAGCAGUA |
| siRNA 5074 | 5074 | ACUGCUAAUAACCUAUAUA | 10564 | UAUAUAGGUUAUUAGCAGU |
| siRNA 5075 | 5075 | CUGCUAAUAACCUAUAUAA | 10565 | UUAUAUAGGUUAUUAGCAG |
| siRNA 5076 | 5076 | UGCUAAUAACCUAUAUAAA | 10566 | UUUAUAUAGGUUAUUAGCA |
| siRNA 5077 | 5077 | GCUAAUAACCUAUAUAAAU | 10567 | AUUUAUAUAGGUUAUUAGC |
| siRNA 5078 | 5078 | CUAAUAACCUAUAUAAAUU | 10568 | AAUUUAUAUAGGUUAUUAG |
| siRNA 5079 | 5079 | UAAUAACCUAUAUAAAUUU | 10569 | AAAUUUAUAUAGGUUAUUA |
| siRNA 5080 | 5080 | AAUAACCUAUAUAAAUUUA | 10570 | UAAAUUUAUAUAGGUUAUU |
| siRNA 5081 | 5081 | AUAACCUAUAUAAAUUUAA | 10571 | UUAAAUUUAUAUAGGUUAU |
| siRNA 5082 | 5082 | UAACCUAUAUAAAUUUAAG | 10572 | CUUAAAUUUAUAUAGGUUA |
| siRNA 5083 | 5083 | AACCUAUAUAAAUUUAAGU | 10573 | ACUUAAAUUUAUAUAGGUU |
| siRNA 5084 | 5084 | ACCUAUAUAAAUUUAAGUG | 10574 | CACUUAAAUUUAUAUAGGU |
| siRNA 5085 | 5085 | CCUAUAUAAAUUUAAGUGA | 10575 | UCACUUAAAUUUAUAUAGG |
| siRNA 5086 | 5086 | CUAUAUAAAUUUAAGUGAC | 10576 | GUCACUUAAAUUUAUAUAG |
| siRNA 5087 | 5087 | UAUAUAAAUUUAAGUGACU | 10577 | AGUCACUUAAAUUUAUAUA |
| siRNA 5088 | 5088 | AUAUAAAUUUAAGUGACUU | 10578 | AAGUCACUUAAAUUUAUAU |
| siRNA 5089 | 5089 | UAUAAAUUUAAGUGACUUG | 10579 | CAAGUCACUUAAAUUUAUA |
| siRNA 5090 | 5090 | AUAAAUUUAAGUGACUUGA | 10580 | UCAAGUCACUUAAAUUUAU |
| siRNA 5091 | 5091 | UAAAUUUAAGUGACUUGAG | 10581 | CUCAAGUCACUUAAAUUUA |
| siRNA 5092 | 5092 | AAAUUUAAGUGACUUGAGG | 10582 | CCUCAAGUCACUUAAAUUU |
| siRNA 5093 | 5093 | AAUUUAAGUGACUUGAGGC | 10583 | GCCUCAAGUCACUUAAAUU |
| siRNA 5094 | 5094 | AUUUAAGUGACUUGAGGCC | 10584 | GGCCUCAAGUCACUUAAAU |
| siRNA 5095 | 5095 | UUUAAGUGACUUGAGGCCU | 10585 | AGGCCUCAAGUCACUUAAA |
| siRNA 5096 | 5096 | UUAAGUGACUUGAGGCCUA | 10586 | UAGGCCUCAAGUCACUUAA |
| siRNA 5097 | 5097 | UAAGUGACUUGAGGCCUAU | 10587 | AUAGGCCUCAAGUCACUUA |
| siRNA 5098 | 5098 | AAGUGACUUGAGGCCUAUA | 10588 | UAUAGGCCUCAAGUCACUU |
| siRNA 5099 | 5099 | AGUGACUUGAGGCCUAUAA | 10589 | UUAUAGGCCUCAAGUCACU |
| siRNA 5100 | 5100 | GUGACUUGAGGCCUAUAAU | 10590 | AUUAUAGGCCUCAAGUCAC |
| siRNA 5101 | 5101 | UGACUUGAGGCCUAUAAUA | 10591 | UAUUAUAGGCCUCAAGUCA |
| siRNA 5102 | 5102 | GACUUGAGGCCUAUAAUAC | 10592 | GUAUUAUAGGCCUCAAGUC |
| siRNA 5103 | 5103 | ACUUGAGGCCUAUAAUACA | 10593 | UGUAUUAUAGGCCUCAAGU |
| siRNA 5104 | 5104 | CUUGAGGCCUAUAAUACAA | 10594 | UUGUAUUAUAGGCCUCAAG |
| siRNA 5105 | 5105 | UUGAGGCCUAUAAUACAAU | 10595 | AUUGUAUUAUAGGCCUCAA |
| siRNA 5106 | 5106 | UGAGGCCUAUAAUACAAUC | 10596 | GAUUGUAUUAUAGGCCUCA |
| siRNA 5107 | 5107 | GAGGCCUAUAAUACAAUCU | 10597 | AGAUUGUAUUAUAGGCCUC |
| siRNA 5108 | 5108 | AGGCCUAUAAUACAAUCUG | 10598 | CAGAUUGUAUUAUAGGCCU |
| siRNA 5109 | 5109 | GGCCUAUAAUACAAUCUGC | 10599 | GCAGAUUGUAUUAUAGGCC |
| siRNA 5110 | 5110 | GCCUAUAAUACAAUCUGCU | 10600 | AGCAGAUUGUAUUAUAGGC |
| siRNA 5111 | 5111 | CCUAUAAUACAAUCUGCUA | 10601 | UAGCAGAUUGUAUUAUAGG |
| siRNA 5112 | 5112 | CUAUAAUACAAUCUGCUAC | 10602 | GUAGCAGAUUGUAUUAUAG |
| siRNA 5113 | 5113 | UAUAAUACAAUCUGCUACU | 10603 | AGUAGCAGAUUGUAUUAUA |
| siRNA 5114 | 5114 | AUAAUACAAUCUGCUACUU | 10604 | AAGUAGCAGAUUGUAUUAU |
| siRNA 5115 | 5115 | UAAUACAAUCUGCUACUUU | 10605 | AAAGUAGCAGAUUGUAUUA |
| siRNA 5116 | 5116 | AAUACAAUCUGCUACUUUA | 10606 | UAAAGUAGCAGAUUGUAUU |
| siRNA 5117 | 5117 | AUACAAUCUGCUACUUUAC | 10607 | GUAAAGUAGCAGAUUGUAU |
| siRNA 5118 | 5118 | UACAAUCUGCUACUUUACU | 10608 | AGUAAAGUAGCAGAUUGUA |
| siRNA 5119 | 5119 | ACAAUCUGCUACUUUACUA | 10609 | UAGUAAAGUAGCAGAUUGU |
| siRNA 5120 | 5120 | CAAUCUGCUACUUUACUAA | 10610 | UUAGUAAAGUAGCAGAUUG |
| siRNA 5121 | 5121 | AAUCUGCUACUUUACUAAU | 10611 | AUUAGUAAAGUAGCAGAUU |
| siRNA 5122 | 5122 | AUCUGCUACUUUACUAAUU | 10612 | AAUUAGUAAAGUAGCAGAU |
| siRNA 5123 | 5123 | UCUGCUACUUUACUAAUUC | 10613 | GAAUUAGUAAAGUAGCAGA |
| siRNA 5124 | 5124 | CUGCUACUUUACUAAUUCA | 10614 | UGAAUUAGUAAAGUAGCAG |
| siRNA 5125 | 5125 | UGCUACUUUACUAAUUCAU | 10615 | AUGAAUUAGUAAAGUAGCA |
| siRNA 5126 | 5126 | GCUACUUUACUAAUUCAUA | 10616 | UAUGAAUUAGUAAAGUAGC |
| siRNA 5127 | 5127 | CUACUUUACUAAUUCAUAA | 10617 | UUAUGAAUUAGUAAAGUAG |
| siRNA 5128 | 5128 | UACUUUACUAAUUCAUAAA | 10618 | UUUAUGAAUUAGUAAAGUA |
| siRNA 5129 | 5129 | ACUUUACUAAUUCAUAAAU | 10619 | AUUUAUGAAUUAGUAAAGU |
| siRNA 5130 | 5130 | CUUUACUAAUUCAUAAAUU | 10620 | AAUUUAUGAAUUAGUAAAG |
| siRNA 5131 | 5131 | UUUACUAAUUCAUAAAUUC | 10621 | GAAUUUAUGAAUUAGUAAA |
| siRNA 5132 | 5132 | UUACUAAUUCAUAAAUUCA | 10622 | UGAAUUUAUGAAUUAGUAA |
| siRNA 5133 | 5133 | UACUAAUUCAUAAAUUCAA | 10623 | UUGAAUUUAUGAAUUAGUA |
| siRNA 5134 | 5134 | ACUAAUUCAUAAAUUCAAA | 10624 | UUUGAAUUUAUGAAUUAGU |
| siRNA 5135 | 5135 | CUAAUUCAUAAAUUCAAAA | 10625 | UUUUGAAUUUAUGAAUUAG |
| siRNA 5136 | 5136 | UAAUUCAUAAAUUCAAAAA | 10626 | UUUUUGAAUUUAUGAAUUA |
| siRNA 5137 | 5137 | AAUUCAUAAAUUCAAAAAA | 10627 | UUUUUUGAAUUUAUGAAUU |
| siRNA 5138 | 5138 | AUUCAUAAAUUCAAAAAAA | 10628 | UUUUUUUGAAUUUAUGAAU |
| siRNA 5139 | 5139 | UUCAUAAAUUCAAAAAAAA | 10629 | UUUUUUUUGAAUUUAUGAA |
| siRNA 5140 | 5140 | UCAUAAAUUCAAAAAAAAU | 10630 | AUUUUUUUUGAAUUUAUGA |
| siRNA 5141 | 5141 | CAUAAAUUCAAAAAAAAUU | 10631 | AAUUUUUUUUGAAUUUAUG |
| siRNA 5142 | 5142 | AUAAAUUCAAAAAAAAUUC | 10632 | GAAUUUUUUUUGAAUUUAU |
| siRNA 5143 | 5143 | UAAAUUCAAAAAAAAUUCU | 10633 | AGAAUUUUUUUUGAAUUUA |
| siRNA 5144 | 5144 | AAAUUCAAAAAAAAUUCUA | 10634 | UAGAAUUUUUUUUGAAUUU |
| siRNA 5145 | 5145 | AAUUCAAAAAAAAUUCUAU | 10635 | AUAGAAUUUUUUUUGAAUU |
| siRNA 5146 | 5146 | AUUCAAAAAAAAUUCUAUG | 10636 | CAUAGAAUUUUUUUUGAAU |
| siRNA 5147 | 5147 | UUCAAAAAAAAUUCUAUGG | 10637 | CCAUAGAAUUUUUUUUGAA |
| siRNA 5148 | 5148 | UCAAAAAAAAUUCUAUGGC | 10638 | GCCAUAGAAUUUUUUUUGA |
| siRNA 5149 | 5149 | CAAAAAAAAUUCUAUGGCA | 10639 | UGCCAUAGAAUUUUUUUUG |
| siRNA 5150 | 5150 | AAAAAAAAUUCUAUGGCAU | 10640 | AUGCCAUAGAAUUUUUUUU |
| siRNA 5151 | 5151 | AAAAAAAUUCUAUGGCAUA | 10641 | UAUGCCAUAGAAUUUUUUU |
| siRNA 5152 | 5152 | AAAAAAUUCUAUGGCAUAG | 10642 | CUAUGCCAUAGAAUUUUUU |
| siRNA 5153 | 5153 | AAAAAUUCUAUGGCAUAGG | 10643 | CCUAUGCCAUAGAAUUUUU |
| siRNA 5154 | 5154 | AAAAUUCUAUGGCAUAGGA | 10644 | UCCUAUGCCAUAGAAUUUU |
| siRNA 5155 | 5155 | AAAUUCUAUGGCAUAGGAA | 10645 | UUCCUAUGCCAUAGAAUUU |
| siRNA 5156 | 5156 | AAUUCUAUGGCAUAGGAAC | 10646 | GUUCCUAUGCCAUAGAAUU |
| siRNA 5157 | 5157 | AUUCUAUGGCAUAGGAACC | 10647 | GGUUCCUAUGCCAUAGAAU |
| siRNA 5158 | 5158 | UUCUAUGGCAUAGGAACCA | 10648 | UGGUUCCUAUGCCAUAGAA |
| siRNA 5159 | 5159 | UCUAUGGCAUAGGAACCAA | 10649 | UUGGUUCCUAUGCCAUAGA |
| siRNA 5160 | 5160 | CUAUGGCAUAGGAACCAAC | 10650 | GUUGGUUCCUAUGCCAUAG |
| siRNA 5161 | 5161 | UAUGGCAUAGGAACCAACU | 10651 | AGUUGGUUCCUAUGCCAUA |
| siRNA 5162 | 5162 | AUGGCAUAGGAACCAACUG | 10652 | CAGUUGGUUCCUAUGCCAU |
| siRNA 5163 | 5163 | UGGCAUAGGAACCAACUGC | 10653 | GCAGUUGGUUCCUAUGCCA |
| siRNA 5164 | 5164 | GGCAUAGGAACCAACUGCC | 10654 | GGCAGUUGGUUCCUAUGCC |
| siRNA 5165 | 5165 | GCAUAGGAACCAACUGCCU | 10655 | AGGCAGUUGGUUCCUAUGC |
| siRNA 5166 | 5166 | CAUAGGAACCAACUGCCUU | 10656 | AAGGCAGUUGGUUCCUAUG |
| siRNA 5167 | 5167 | AUAGGAACCAACUGCCUUG | 10657 | CAAGGCAGUUGGUUCCUAU |
| siRNA 5168 | 5168 | UAGGAACCAACUGCCUUGC | 10658 | GCAAGGCAGUUGGUUCCUA |
| siRNA 5169 | 5169 | AGGAACCAACUGCCUUGCC | 10659 | GGCAAGGCAGUUGGUUCCU |
| siRNA 5170 | 5170 | GGAACCAACUGCCUUGCCU | 10660 | AGGCAAGGCAGUUGGUUCC |
| siRNA 5171 | 5171 | GAACCAACUGCCUUGCCUU | 10661 | AAGGCAAGGCAGUUGGUUC |
| siRNA 5172 | 5172 | AACCAACUGCCUUGCCUUC | 10662 | GAAGGCAAGGCAGUUGGUU |
| siRNA 5173 | 5173 | ACCAACUGCCUUGCCUUCA | 10663 | UGAAGGCAAGGCAGUUGGU |
| siRNA 5174 | 5174 | CCAACUGCCUUGCCUUCAA | 10664 | UUGAAGGCAAGGCAGUUGG |
| siRNA 5175 | 5175 | CAACUGCCUUGCCUUCAAG | 10665 | CUUGAAGGCAAGGCAGUUG |
| siRNA 5176 | 5176 | AACUGCCUUGCCUUCAAGA | 10666 | UCUUGAAGGCAAGGCAGUU |
| siRNA 5177 | 5177 | ACUGCCUUGCCUUCAAGAC | 10667 | GUCUUGAAGGCAAGGCAGU |
| siRNA 5178 | 5178 | CUGCCUUGCCUUCAAGACC | 10668 | GGUCUUGAAGGCAAGGCAG |
| siRNA 5179 | 5179 | UGCCUUGCCUUCAAGACCU | 10669 | AGGUCUUGAAGGCAAGGCA |
| siRNA 5180 | 5180 | GCCUUGCCUUCAAGACCUA | 10670 | UAGGUCUUGAAGGCAAGGC |
| siRNA 5181 | 5181 | CCUUGCCUUCAAGACCUAG | 10671 | CUAGGUCUUGAAGGCAAGG |
| siRNA 5182 | 5182 | CUUGCCUUCAAGACCUAGU | 10672 | ACUAGGUCUUGAAGGCAAG |
| siRNA 5183 | 5183 | UUGCCUUCAAGACCUAGUA | 10673 | UACUAGGUCUUGAAGGCAA |
| siRNA 5184 | 5184 | UGCCUUCAAGACCUAGUAA | 10674 | UUACUAGGUCUUGAAGGCA |
| siRNA 5185 | 5185 | GCCUUCAAGACCUAGUAAC | 10675 | GUUACUAGGUCUUGAAGGC |
| siRNA 5186 | 5186 | CCUUCAAGACCUAGUAACU | 10676 | AGUUACUAGGUCUUGAAGG |
| siRNA 5187 | 5187 | CUUCAAGACCUAGUAACUU | 10677 | AAGUUACUAGGUCUUGAAG |
| siRNA 5188 | 5188 | UUCAAGACCUAGUAACUUU | 10678 | AAAGUUACUAGGUCUUGAA |
| siRNA 5189 | 5189 | UCAAGACCUAGUAACUUUC | 10679 | GAAAGUUACUAGGUCUUGA |
| siRNA 5190 | 5190 | CAAGACCUAGUAACUUUCU | 10680 | AGAAAGUUACUAGGUCUUG |
| siRNA 5191 | 5191 | AAGACCUAGUAACUUUCUC | 10681 | GAGAAAGUUACUAGGUCUU |
| siRNA 5192 | 5192 | AGACCUAGUAACUUUCUCU | 10682 | AGAGAAAGUUACUAGGUCU |
| siRNA 5193 | 5193 | GACCUAGUAACUUUCUCUA | 10683 | UAGAGAAAGUUACUAGGUC |
| siRNA 5194 | 5194 | ACCUAGUAACUUUCUCUAU | 10684 | AUAGAGAAAGUUACUAGGU |
| siRNA 5195 | 5195 | CCUAGUAACUUUCUCUAUA | 10685 | UAUAGAGAAAGUUACUAGG |
| siRNA 5196 | 5196 | CUAGUAACUUUCUCUAUAA | 10686 | UUAUAGAGAAAGUUACUAG |
| siRNA 5197 | 5197 | UAGUAACUUUCUCUAUAAA | 10687 | UUUAUAGAGAAAGUUACUA |
| siRNA 5198 | 5198 | AGUAACUUUCUCUAUAAAU | 10688 | AUUUAUAGAGAAAGUUACU |
| siRNA 5199 | 5199 | GUAACUUUCUCUAUAAAUC | 10689 | GAUUUAUAGAGAAAGUUAC |
| siRNA 5200 | 5200 | UAACUUUCUCUAUAAAUCU | 10690 | AGAUUUAUAGAGAAAGUUA |
| siRNA 5201 | 5201 | AACUUUCUCUAUAAAUCUC | 10691 | GAGAUUUAUAGAGAAAGUU |
| siRNA 5202 | 5202 | ACUUUCUCUAUAAAUCUCG | 10692 | CGAGAUUUAUAGAGAAAGU |
| siRNA 5203 | 5203 | CUUUCUCUAUAAAUCUCGU | 10693 | ACGAGAUUUAUAGAGAAAG |
| siRNA 5204 | 5204 | UUUCUCUAUAAAUCUCGUG | 10694 | CACGAGAUUUAUAGAGAAA |
| siRNA 5205 | 5205 | UUCUCUAUAAAUCUCGUGU | 10695 | ACACGAGAUUUAUAGAGAA |
| siRNA 5206 | 5206 | UCUCUAUAAAUCUCGUGUU | 10696 | AACACGAGAUUUAUAGAGA |
| siRNA 5207 | 5207 | CUCUAUAAAUCUCGUGUUA | 10697 | UAACACGAGAUUUAUAGAG |
| siRNA 5208 | 5208 | UCUAUAAAUCUCGUGUUAA | 10698 | UUAACACGAGAUUUAUAGA |
| siRNA 5209 | 5209 | CUAUAAAUCUCGUGUUAAC | 10699 | GUUAACACGAGAUUUAUAG |
| siRNA 5210 | 5210 | UAUAAAUCUCGUGUUAACU | 10700 | AGUUAACACGAGAUUUAUA |
| siRNA 5211 | 5211 | AUAAAUCUCGUGUUAACUG | 10701 | CAGUUAACACGAGAUUUAU |
| siRNA 5212 | 5212 | UAAAUCUCGUGUUAACUGA | 10702 | UCAGUUAACACGAGAUUUA |
| siRNA 5213 | 5213 | AAAUCUCGUGUUAACUGAA | 10703 | UUCAGUUAACACGAGAUUU |
| siRNA 5214 | 5214 | AAUCUCGUGUUAACUGAAA | 10704 | UUUCAGUUAACACGAGAUU |
| siRNA 5215 | 5215 | AUCUCGUGUUAACUGAAAU | 10705 | AUUUCAGUUAACACGAGAU |
| siRNA 5216 | 5216 | UCUCGUGUUAACUGAAAUU | 10706 | AAUUUCAGUUAACACGAGA |
| siRNA 5217 | 5217 | CUCGUGUUAACUGAAAUUU | 10707 | AAAUUUCAGUUAACACGAG |
| siRNA 5218 | 5218 | UCGUGUUAACUGAAAUUUU | 10708 | AAAAUUUCAGUUAACACGA |
| siRNA 5219 | 5219 | CGUGUUAACUGAAAUUUUU | 10709 | AAAAAUUUCAGUUAACACG |
| siRNA 5220 | 5220 | GUGUUAACUGAAAUUUUUU | 10710 | AAAAAAUUUCAGUUAACAC |
| siRNA 5221 | 5221 | UGUUAACUGAAAUUUUUUU | 10711 | AAAAAAAUUUCAGUUAACA |
| siRNA 5222 | 5222 | GUUAACUGAAAUUUUUUUU | 10712 | AAAAAAAAUUUCAGUUAAC |
| siRNA 5223 | 5223 | UUAACUGAAAUUUUUUUUA | 10713 | UAAAAAAAAUUUCAGUUAA |
| siRNA 5224 | 5224 | UAACUGAAAUUUUUUUUAA | 10714 | UUAAAAAAAAUUUCAGUUA |
| siRNA 5225 | 5225 | AACUGAAAUUUUUUUUAAA | 10715 | UUUAAAAAAAAUUUCAGUU |
| siRNA 5226 | 5226 | ACUGAAAUUUUUUUUAAAU | 10716 | AUUUAAAAAAAAUUUCAGU |
| siRNA 5227 | 5227 | CUGAAAUUUUUUUUAAAUA | 10717 | UAUUUAAAAAAAAUUUCAG |
| siRNA 5228 | 5228 | UGAAAUUUUUUUUAAAUAU | 10718 | AUAUUUAAAAAAAAUUUCA |
| siRNA 5229 | 5229 | GAAAUUUUUUUUAAAUAUA | 10719 | UAUAUUUAAAAAAAAUUUC |
| siRNA 5230 | 5230 | AAAUUUUUUUUAAAUAUAU | 10720 | AUAUAUUUAAAAAAAAUUU |
| siRNA 5231 | 5231 | AAUUUUUUUUAAAUAUAUU | 10721 | AAUAUAUUUAAAAAAAAUU |
| siRNA 5232 | 5232 | AUUUUUUUUAAAUAUAUUU | 10722 | AAAUAUAUUUAAAAAAAAU |
| siRNA 5233 | 5233 | UUUUUUUUAAAUAUAUUUU | 10723 | AAAAUAUAUUUAAAAAAAA |
| siRNA 5234 | 5234 | UUUUUUUAAAUAUAUUUUU | 10724 | AAAAAUAUAUUUAAAAAAA |
| siRNA 5235 | 5235 | UUUUUUAAAUAUAUUUUUU | 10725 | AAAAAAUAUAUUUAAAAAA |
| siRNA 5236 | 5236 | UUUUUAAAUAUAUUUUUUA | 10726 | UAAAAAAUAUAUUUAAAAA |
| siRNA 5237 | 5237 | UUUUAAAUAUAUUUUUUAG | 10727 | CUAAAAAAUAUAUUUAAAA |
| siRNA 5238 | 5238 | UUUAAAUAUAUUUUUUAGA | 10728 | UCUAAAAAAUAUAUUUAAA |
| siRNA 5239 | 5239 | UUAAAUAUAUUUUUUAGAU | 10729 | AUCUAAAAAAUAUAUUUAA |
| siRNA 5240 | 5240 | UAAAUAUAUUUUUUAGAUU | 10730 | AAUCUAAAAAAUAUAUUUA |
| siRNA 5241 | 5241 | AAAUAUAUUUUUUAGAUUG | 10731 | CAAUCUAAAAAAUAUAUUU |
| siRNA 5242 | 5242 | AAUAUAUUUUUUAGAUUGG | 10732 | CCAAUCUAAAAAAUAUAUU |
| siRNA 5243 | 5243 | AUAUAUUUUUUAGAUUGGU | 10733 | ACCAAUCUAAAAAAUAUAU |
| siRNA 5244 | 5244 | UAUAUUUUUUAGAUUGGUA | 10734 | UACCAAUCUAAAAAAUAUA |
| siRNA 5245 | 5245 | AUAUUUUUUAGAUUGGUAA | 10735 | UUACCAAUCUAAAAAAUAU |
| siRNA 5246 | 5246 | UAUUUUUUAGAUUGGUAAU | 10736 | AUUACCAAUCUAAAAAAUA |
| siRNA 5247 | 5247 | AUUUUUUAGAUUGGUAAUA | 10737 | UAUUACCAAUCUAAAAAAU |
| siRNA 5248 | 5248 | UUUUUUAGAUUGGUAAUAU | 10738 | AUAUUACCAAUCUAAAAAA |
| siRNA 5249 | 5249 | UUUUUAGAUUGGUAAUAUU | 10739 | AAUAUUACCAAUCUAAAAA |
| siRNA 5250 | 5250 | UUUUAGAUUGGUAAUAUUU | 10740 | AAAUAUUACCAAUCUAAAA |
| siRNA 5251 | 5251 | UUUAGAUUGGUAAUAUUUA | 10741 | UAAAUAUUACCAAUCUAAA |
| siRNA 5252 | 5252 | UUAGAUUGGUAAUAUUUAA | 10742 | UUAAAUAUUACCAAUCUAA |
| siRNA 5253 | 5253 | UAGAUUGGUAAUAUUUAAA | 10743 | UUUAAAUAUUACCAAUCUA |
| siRNA 5254 | 5254 | AGAUUGGUAAUAUUUAAAC | 10744 | GUUUAAAUAUUACCAAUCU |
| siRNA 5255 | 5255 | GAUUGGUAAUAUUUAAACC | 10745 | GGUUUAAAUAUUACCAAUC |
| siRNA 5256 | 5256 | AUUGGUAAUAUUUAAACCA | 10746 | UGGUUUAAAUAUUACCAAU |
| siRNA 5257 | 5257 | UUGGUAAUAUUUAAACCAG | 10747 | CUGGUUUAAAUAUUACCAA |
| siRNA 5258 | 5258 | UGGUAAUAUUUAAACCAGC | 10748 | GCUGGUUUAAAUAUUACCA |
| siRNA 5259 | 5259 | GGUAAUAUUUAAACCAGCA | 10749 | UGCUGGUUUAAAUAUUACC |
| siRNA 5260 | 5260 | GUAAUAUUUAAACCAGCAA | 10750 | UUGCUGGUUUAAAUAUUAC |
| siRNA 5261 | 5261 | UAAUAUUUAAACCAGCAAA | 10751 | UUUGCUGGUUUAAAUAUUA |
| siRNA 5262 | 5262 | AAUAUUUAAACCAGCAAAU | 10752 | AUUUGCUGGUUUAAAUAUU |
| siRNA 5263 | 5263 | AUAUUUAAACCAGCAAAUA | 10753 | UAUUUGCUGGUUUAAAUAU |
| siRNA 5264 | 5264 | UAUUUAAACCAGCAAAUAC | 10754 | GUAUUUGCUGGUUUAAAUA |
| siRNA 5265 | 5265 | AUUUAAACCAGCAAAUACU | 10755 | AGUAUUUGCUGGUUUAAAU |
| siRNA 5266 | 5266 | UUUAAACCAGCAAAUACUU | 10756 | AAGUAUUUGCUGGUUUAAA |
| siRNA 5267 | 5267 | UUAAACCAGCAAAUACUUA | 10757 | UAAGUAUUUGCUGGUUUAA |
| siRNA 5268 | 5268 | UAAACCAGCAAAUACUUAA | 10758 | UUAAGUAUUUGCUGGUUUA |
| siRNA 5269 | 5269 | AAACCAGCAAAUACUUAAA | 10759 | UUUAAGUAUUUGCUGGUUU |
| siRNA 5270 | 5270 | AACCAGCAAAUACUUAAAG | 10760 | CUUUAAGUAUUUGCUGGUU |
| siRNA 5271 | 5271 | ACCAGCAAAUACUUAAAGC | 10761 | GCUUUAAGUAUUUGCUGGU |
| siRNA 5272 | 5272 | CCAGCAAAUACUUAAAGCU | 10762 | AGCUUUAAGUAUUUGCUGG |
| siRNA 5273 | 5273 | CAGCAAAUACUUAAAGCUU | 10763 | AAGCUUUAAGUAUUUGCUG |
| siRNA 5274 | 5274 | AGCAAAUACUUAAAGCUUU | 10764 | AAAGCUUUAAGUAUUUGCU |
| siRNA 5275 | 5275 | GCAAAUACUUAAAGCUUUA | 10765 | UAAAGCUUUAAGUAUUUGC |
| siRNA 5276 | 5276 | CAAAUACUUAAAGCUUUAU | 10766 | AUAAAGCUUUAAGUAUUUG |
| siRNA 5277 | 5277 | AAAUACUUAAAGCUUUAUU | 10767 | AAUAAAGCUUUAAGUAUUU |
| siRNA 5278 | 5278 | AAUACUUAAAGCUUUAUUA | 10768 | UAAUAAAGCUUUAAGUAUU |
| siRNA 5279 | 5279 | AUACUUAAAGCUUUAUUAA | 10769 | UUAAUAAAGCUUUAAGUAU |
| siRNA 5280 | 5280 | UACUUAAAGCUUUAUUAAA | 10770 | UUUAAUAAAGCUUUAAGUA |
| siRNA 5281 | 5281 | ACUUAAAGCUUUAUUAAAC | 10771 | GUUUAAUAAAGCUUUAAGU |
| siRNA 5282 | 5282 | CUUAAAGCUUUAUUAAACA | 10772 | UGUUUAAUAAAGCUUUAAG |
| siRNA 5283 | 5283 | UUAAAGCUUUAUUAAACAU | 10773 | AUGUUUAAUAAAGCUUUAA |
| siRNA 5284 | 5284 | UAAAGCUUUAUUAAACAUU | 10774 | AAUGUUUAAUAAAGCUUUA |
| siRNA 5285 | 5285 | AAAGCUUUAUUAAACAUUU | 10775 | AAAUGUUUAAUAAAGCUUU |
| siRNA 5286 | 5286 | AAGCUUUAUUAAACAUUUU | 10776 | AAAAUGUUUAAUAAAGCUU |
| siRNA 5287 | 5287 | AGCUUUAUUAAACAUUUUA | 10777 | UAAAAUGUUUAAUAAAGCU |
| siRNA 5288 | 5288 | GCUUUAUUAAACAUUUUAA | 10778 | UUAAAAUGUUUAAUAAAGC |
| siRNA 5289 | 5289 | CUUUAUUAAACAUUUUAAU | 10779 | AUUAAAAUGUUUAAUAAAG |
| siRNA 5290 | 5290 | UUUAUUAAACAUUUUAAUC | 10780 | GAUUAAAAUGUUUAAUAAA |
| siRNA 5291 | 5291 | UUAUUAAACAUUUUAAUCA | 10781 | UGAUUAAAAUGUUUAAUAA |
| siRNA 5292 | 5292 | UAUUAAACAUUUUAAUCAG | 10782 | CUGAUUAAAAUGUUUAAUA |
| siRNA 5293 | 5293 | AUUAAACAUUUUAAUCAGA | 10783 | UCUGAUUAAAAUGUUUAAU |
| siRNA 5294 | 5294 | UUAAACAUUUUAAUCAGAU | 10784 | AUCUGAUUAAAAUGUUUAA |
| siRNA 5295 | 5295 | UAAACAUUUUAAUCAGAUA | 10785 | UAUCUGAUUAAAAUGUUUA |
| siRNA 5296 | 5296 | AAACAUUUUAAUCAGAUAA | 10786 | UUAUCUGAUUAAAAUGUUU |
| siRNA 5297 | 5297 | AACAUUUUAAUCAGAUAAG | 10787 | CUUAUCUGAUUAAAAUGUU |
| siRNA 5298 | 5298 | ACAUUUUAAUCAGAUAAGU | 10788 | ACUUAUCUGAUUAAAAUGU |
| siRNA 5299 | 5299 | CAUUUUAAUCAGAUAAGUG | 10789 | CACUUAUCUGAUUAAAAUG |
| siRNA 5300 | 5300 | AUUUUAAUCAGAUAAGUGA | 10790 | UCACUUAUCUGAUUAAAAU |
| siRNA 5301 | 5301 | UUUUAAUCAGAUAAGUGAG | 10791 | CUCACUUAUCUGAUUAAAA |
| siRNA 5302 | 5302 | UUUAAUCAGAUAAGUGAGU | 10792 | ACUCACUUAUCUGAUUAAA |
| siRNA 5303 | 5303 | UUAAUCAGAUAAGUGAGUA | 10793 | UACUCACUUAUCUGAUUAA |
| siRNA 5304 | 5304 | UAAUCAGAUAAGUGAGUAA | 10794 | UUACUCACUUAUCUGAUUA |
| siRNA 5305 | 5305 | AAUCAGAUAAGUGAGUAAA | 10795 | UUUACUCACUUAUCUGAUU |
| siRNA 5306 | 5306 | AUCAGAUAAGUGAGUAAAG | 10796 | CUUUACUCACUUAUCUGAU |
| siRNA 5307 | 5307 | UCAGAUAAGUGAGUAAAGC | 10797 | GCUUUACUCACUUAUCUGA |
| siRNA 5308 | 5308 | CAGAUAAGUGAGUAAAGCU | 10798 | AGCUUUACUCACUUAUCUG |
| siRNA 5309 | 5309 | AGAUAAGUGAGUAAAGCUU | 10799 | AAGCUUUACUCACUUAUCU |
| siRNA 5310 | 5310 | GAUAAGUGAGUAAAGCUUU | 10800 | AAAGCUUUACUCACUUAUC |
| siRNA 5311 | 5311 | AUAAGUGAGUAAAGCUUUU | 10801 | AAAAGCUUUACUCACUUAU |
| siRNA 5312 | 5312 | UAAGUGAGUAAAGCUUUUA | 10802 | UAAAAGCUUUACUCACUUA |
| siRNA 5313 | 5313 | AAGUGAGUAAAGCUUUUAU | 10803 | AUAAAAGCUUUACUCACUU |
| siRNA 5314 | 5314 | AGUGAGUAAAGCUUUUAUU | 10804 | AAUAAAAGCUUUACUCACU |
| siRNA 5315 | 5315 | GUGAGUAAAGCUUUUAUUU | 10805 | AAAUAAAAGCUUUACUCAC |
| siRNA 5316 | 5316 | UGAGUAAAGCUUUUAUUUG | 10806 | CAAAUAAAAGCUUUACUCA |
| siRNA 5317 | 5317 | GAGUAAAGCUUUUAUUUGC | 10807 | GCAAAUAAAAGCUUUACUC |
| siRNA 5318 | 5318 | AGUAAAGCUUUUAUUUGCC | 10808 | GGCAAAUAAAAGCUUUACU |
| siRNA 5319 | 5319 | GUAAAGCUUUUAUUUGCCA | 10809 | UGGCAAAUAAAAGCUUUAC |
| siRNA 5320 | 5320 | UAAAGCUUUUAUUUGCCAU | 10810 | AUGGCAAAUAAAAGCUUUA |
| siRNA 5321 | 5321 | AAAGCUUUUAUUUGCCAUU | 10811 | AAUGGCAAAUAAAAGCUUU |
| siRNA 5322 | 5322 | AAGCUUUUAUUUGCCAUUU | 10812 | AAAUGGCAAAUAAAAGCUU |
| siRNA 5323 | 5323 | AGCUUUUAUUUGCCAUUUG | 10813 | CAAAUGGCAAAUAAAAGCU |
| siRNA 5324 | 5324 | GCUUUUAUUUGCCAUUUGG | 10814 | CCAAAUGGCAAAUAAAAGC |
| siRNA 5325 | 5325 | CUUUUAUUUGCCAUUUGGA | 10815 | UCCAAAUGGCAAAUAAAAG |
| siRNA 5326 | 5326 | UUUUAUUUGCCAUUUGGAU | 10816 | AUCCAAAUGGCAAAUAAAA |
| siRNA 5327 | 5327 | UUUAUUUGCCAUUUGGAUG | 10817 | CAUCCAAAUGGCAAAUAAA |
| siRNA 5328 | 5328 | UUAUUUGCCAUUUGGAUGC | 10818 | GCAUCCAAAUGGCAAAUAA |
| siRNA 5329 | 5329 | UAUUUGCCAUUUGGAUGCC | 10819 | GGCAUCCAAAUGGCAAAUA |
| siRNA 5330 | 5330 | AUUUGCCAUUUGGAUGCCU | 10820 | AGGCAUCCAAAUGGCAAAU |
| siRNA 5331 | 5331 | UUUGCCAUUUGGAUGCCUU | 10821 | AAGGCAUCCAAAUGGCAAA |
| siRNA 5332 | 5332 | UUGCCAUUUGGAUGCCUUC | 10822 | GAAGGCAUCCAAAUGGCAA |
| siRNA 5333 | 5333 | UGCCAUUUGGAUGCCUUCG | 10823 | CGAAGGCAUCCAAAUGGCA |
| siRNA 5334 | 5334 | GCCAUUUGGAUGCCUUCGU | 10824 | ACGAAGGCAUCCAAAUGGC |
| siRNA 5335 | 5335 | CCAUUUGGAUGCCUUCGUU | 10825 | AACGAAGGCAUCCAAAUGG |
| siRNA 5336 | 5336 | CAUUUGGAUGCCUUCGUUC | 10826 | GAACGAAGGCAUCCAAAUG |
| siRNA 5337 | 5337 | AUUUGGAUGCCUUCGUUCA | 10827 | UGAACGAAGGCAUCCAAAU |
| siRNA 5338 | 5338 | UUUGGAUGCCUUCGUUCAA | 10828 | UUGAACGAAGGCAUCCAAA |
| siRNA 5339 | 5339 | UUGGAUGCCUUCGUUCAAA | 10829 | UUUGAACGAAGGCAUCCAA |
| siRNA 5340 | 5340 | UGGAUGCCUUCGUUCAAAG | 10830 | CUUUGAACGAAGGCAUCCA |
| siRNA 5341 | 5341 | GGAUGCCUUCGUUCAAAGU | 10831 | ACUUUGAACGAAGGCAUCC |
| siRNA 5342 | 5342 | GAUGCCUUCGUUCAAAGUG | 10832 | CACUUUGAACGAAGGCAUC |
| siRNA 5343 | 5343 | AUGCCUUCGUUCAAAGUGA | 10833 | UCACUUUGAACGAAGGCAU |
| siRNA 5344 | 5344 | UGCCUUCGUUCAAAGUGAU | 10834 | AUCACUUUGAACGAAGGCA |
| siRNA 5345 | 5345 | GCCUUCGUUCAAAGUGAUA | 10835 | UAUCACUUUGAACGAAGGC |
| siRNA 5346 | 5346 | CCUUCGUUCAAAGUGAUAG | 10836 | CUAUCACUUUGAACGAAGG |
| siRNA 5347 | 5347 | CUUCGUUCAAAGUGAUAGA | 10837 | UCUAUCACUUUGAACGAAG |
| siRNA 5348 | 5348 | UUCGUUCAAAGUGAUAGAG | 10838 | CUCUAUCACUUUGAACGAA |
| siRNA 5349 | 5349 | UCGUUCAAAGUGAUAGAGU | 10839 | ACUCUAUCACUUUGAACGA |
| siRNA 5350 | 5350 | CGUUCAAAGUGAUAGAGUG | 10840 | CACUCUAUCACUUUGAACG |
| siRNA 5351 | 5351 | GUUCAAAGUGAUAGAGUGU | 10841 | ACACUCUAUCACUUUGAAC |
| siRNA 5352 | 5352 | UUCAAAGUGAUAGAGUGUU | 10842 | AACACUCUAUCACUUUGAA |
| siRNA 5353 | 5353 | UCAAAGUGAUAGAGUGUUU | 10843 | AAACACUCUAUCACUUUGA |
| siRNA 5354 | 5354 | CAAAGUGAUAGAGUGUUUU | 10844 | AAAACACUCUAUCACUUUG |
| siRNA 5355 | 5355 | AAAGUGAUAGAGUGUUUUG | 10845 | CAAAACACUCUAUCACUUU |
| siRNA 5356 | 5356 | AAGUGAUAGAGUGUUUUGC | 10846 | GCAAAACACUCUAUCACUU |
| siRNA 5357 | 5357 | AGUGAUAGAGUGUUUUGCU | 10847 | AGCAAAACACUCUAUCACU |
| siRNA 5358 | 5358 | GUGAUAGAGUGUUUUGCUG | 10848 | CAGCAAAACACUCUAUCAC |
| siRNA 5359 | 5359 | UGAUAGAGUGUUUUGCUGA | 10849 | UCAGCAAAACACUCUAUCA |
| siRNA 5360 | 5360 | GAUAGAGUGUUUUGCUGAU | 10850 | AUCAGCAAAACACUCUAUC |
| siRNA 5361 | 5361 | AUAGAGUGUUUUGCUGAUA | 10851 | UAUCAGCAAAACACUCUAU |
| siRNA 5362 | 5362 | UAGAGUGUUUUGCUGAUAG | 10852 | CUAUCAGCAAAACACUCUA |
| siRNA 5363 | 5363 | AGAGUGUUUUGCUGAUAGU | 10853 | ACUAUCAGCAAAACACUCU |
| siRNA 5364 | 5364 | GAGUGUUUUGCUGAUAGUG | 10854 | CACUAUCAGCAAAACACUC |
| siRNA 5365 | 5365 | AGUGUUUUGCUGAUAGUGC | 10855 | GCACUAUCAGCAAAACACU |
| siRNA 5366 | 5366 | GUGUUUUGCUGAUAGUGCU | 10856 | AGCACUAUCAGCAAAACAC |
| siRNA 5367 | 5367 | UGUUUUGCUGAUAGUGCUG | 10857 | CAGCACUAUCAGCAAAACA |
| siRNA 5368 | 5368 | GUUUUGCUGAUAGUGCUGU | 10858 | ACAGCACUAUCAGCAAAAC |
| siRNA 5369 | 5369 | UUUUGCUGAUAGUGCUGUA | 10859 | UACAGCACUAUCAGCAAAA |
| siRNA 5370 | 5370 | UUUGCUGAUAGUGCUGUAG | 10860 | CUACAGCACUAUCAGCAAA |
| siRNA 5371 | 5371 | UUGCUGAUAGUGCUGUAGC | 10861 | GCUACAGCACUAUCAGCAA |
| siRNA 5372 | 5372 | UGCUGAUAGUGCUGUAGCA | 10862 | UGCUACAGCACUAUCAGCA |
| siRNA 5373 | 5373 | GCUGAUAGUGCUGUAGCAG | 10863 | CUGCUACAGCACUAUCAGC |
| siRNA 5374 | 5374 | CUGAUAGUGCUGUAGCAGC | 10864 | GCUGCUACAGCACUAUCAG |
| siRNA 5375 | 5375 | UGAUAGUGCUGUAGCAGCA | 10865 | UGCUGCUACAGCACUAUCA |
| siRNA 5376 | 5376 | GAUAGUGCUGUAGCAGCAG | 10866 | CUGCUGCUACAGCACUAUC |
| siRNA 5377 | 5377 | AUAGUGCUGUAGCAGCAGU | 10867 | ACUGCUGCUACAGCACUAU |
| siRNA 5378 | 5378 | UAGUGCUGUAGCAGCAGUU | 10868 | AACUGCUGCUACAGCACUA |
| siRNA 5379 | 5379 | AGUGCUGUAGCAGCAGUUG | 10869 | CAACUGCUGCUACAGCACU |
| siRNA 5380 | 5380 | GUGCUGUAGCAGCAGUUGU | 10870 | ACAACUGCUGCUACAGCAC |
| siRNA 5381 | 5381 | UGCUGUAGCAGCAGUUGUA | 10871 | UACAACUGCUGCUACAGCA |
| siRNA 5382 | 5382 | GCUGUAGCAGCAGUUGUAA | 10872 | UUACAACUGCUGCUACAGC |
| siRNA 5383 | 5383 | CUGUAGCAGCAGUUGUAAA | 10873 | UUUACAACUGCUGCUACAG |
| siRNA 5384 | 5384 | UGUAGCAGCAGUUGUAAAG | 10874 | CUUUACAACUGCUGCUACA |
| siRNA 5385 | 5385 | GUAGCAGCAGUUGUAAAGU | 10875 | ACUUUACAACUGCUGCUAC |
| siRNA 5386 | 5386 | UAGCAGCAGUUGUAAAGUA | 10876 | UACUUUACAACUGCUGCUA |
| siRNA 5387 | 5387 | AGCAGCAGUUGUAAAGUAG | 10877 | CUACUUUACAACUGCUGCU |
| siRNA 5388 | 5388 | GCAGCAGUUGUAAAGUAGC | 10878 | GCUACUUUACAACUGCUGC |
| siRNA 5389 | 5389 | CAGCAGUUGUAAAGUAGCC | 10879 | GGCUACUUUACAACUGCUG |
| siRNA 5390 | 5390 | AGCAGUUGUAAAGUAGCCA | 10880 | UGGCUACUUUACAACUGCU |
| siRNA 5391 | 5391 | GCAGUUGUAAAGUAGCCAA | 10881 | UUGGCUACUUUACAACUGC |
| siRNA 5392 | 5392 | CAGUUGUAAAGUAGCCAAA | 10882 | UUUGGCUACUUUACAACUG |
| siRNA 5393 | 5393 | AGUUGUAAAGUAGCCAAAA | 10883 | UUUUGGCUACUUUACAACU |
| siRNA 5394 | 5394 | GUUGUAAAGUAGCCAAAAG | 10884 | CUUUUGGCUACUUUACAAC |
| siRNA 5395 | 5395 | UUGUAAAGUAGCCAAAAGC | 10885 | GCUUUUGGCUACUUUACAA |
| siRNA 5396 | 5396 | UGUAAAGUAGCCAAAAGCC | 10886 | GGCUUUUGGCUACUUUACA |
| siRNA 5397 | 5397 | GUAAAGUAGCCAAAAGCCA | 10887 | UGGCUUUUGGCUACUUUAC |
| siRNA 5398 | 5398 | UAAAGUAGCCAAAAGCCAC | 10888 | GUGGCUUUUGGCUACUUUA |
| siRNA 5399 | 5399 | AAAGUAGCCAAAAGCCACG | 10889 | CGUGGCUUUUGGCUACUUU |
| siRNA 5400 | 5400 | AAGUAGCCAAAAGCCACGU | 10890 | ACGUGGCUUUUGGCUACUU |
| siRNA 5401 | 5401 | AGUAGCCAAAAGCCACGUU | 10891 | AACGUGGCUUUUGGCUACU |
| siRNA 5402 | 5402 | GUAGCCAAAAGCCACGUUG | 10892 | CAACGUGGCUUUUGGCUAC |
| siRNA 5403 | 5403 | UAGCCAAAAGCCACGUUGU | 10893 | ACAACGUGGCUUUUGGCUA |
| siRNA 5404 | 5404 | AGCCAAAAGCCACGUUGUU | 10894 | AACAACGUGGCUUUUGGCU |
| siRNA 5405 | 5405 | GCCAAAAGCCACGUUGUUU | 10895 | AAACAACGUGGCUUUUGGC |
| siRNA 5406 | 5406 | CCAAAAGCCACGUUGUUUA | 10896 | UAAACAACGUGGCUUUUGG |
| siRNA 5407 | 5407 | CAAAAGCCACGUUGUUUAU | 10897 | AUAAACAACGUGGCUUUUG |
| siRNA 5408 | 5408 | AAAAGCCACGUUGUUUAUU | 10898 | AAUAAACAACGUGGCUUUU |
| siRNA 5409 | 5409 | AAAGCCACGUUGUUUAUUU | 10899 | AAAUAAACAACGUGGCUUU |
| siRNA 5410 | 5410 | AAGCCACGUUGUUUAUUUA | 10900 | UAAAUAAACAACGUGGCUU |
| siRNA 5411 | 5411 | AGCCACGUUGUUUAUUUAC | 10901 | GUAAAUAAACAACGUGGCU |
| siRNA 5412 | 5412 | GCCACGUUGUUUAUUUACU | 10902 | AGUAAAUAAACAACGUGGC |
| siRNA 5413 | 5413 | CCACGUUGUUUAUUUACUG | 10903 | CAGUAAAUAAACAACGUGG |
| siRNA 5414 | 5414 | CACGUUGUUUAUUUACUGG | 10904 | CCAGUAAAUAAACAACGUG |
| siRNA 5415 | 5415 | ACGUUGUUUAUUUACUGGU | 10905 | ACCAGUAAAUAAACAACGU |
| siRNA 5416 | 5416 | CGUUGUUUAUUUACUGGUU | 10906 | AACCAGUAAAUAAACAACG |
| siRNA 5417 | 5417 | GUUGUUUAUUUACUGGUUU | 10907 | AAACCAGUAAAUAAACAAC |
| siRNA 5418 | 5418 | UUGUUUAUUUACUGGUUUG | 10908 | CAAACCAGUAAAUAAACAA |
| siRNA 5419 | 5419 | UGUUUAUUUACUGGUUUGU | 10909 | ACAAACCAGUAAAUAAACA |
| siRNA 5420 | 5420 | GUUUAUUUACUGGUUUGUG | 10910 | CACAAACCAGUAAAUAAAC |
| siRNA 5421 | 5421 | UUUAUUUACUGGUUUGUGG | 10911 | CCACAAACCAGUAAAUAAA |
| siRNA 5422 | 5422 | UUAUUUACUGGUUUGUGGC | 10912 | GCCACAAACCAGUAAAUAA |
| siRNA 5423 | 5423 | UAUUUACUGGUUUGUGGCC | 10913 | GGCCACAAACCAGUAAAUA |
| siRNA 5424 | 5424 | AUUUACUGGUUUGUGGCCU | 10914 | AGGCCACAAACCAGUAAAU |
| siRNA 5425 | 5425 | UUUACUGGUUUGUGGCCUU | 10915 | AAGGCCACAAACCAGUAAA |
| siRNA 5426 | 5426 | UUACUGGUUUGUGGCCUUU | 10916 | AAAGGCCACAAACCAGUAA |
| siRNA 5427 | 5427 | UACUGGUUUGUGGCCUUUU | 10917 | AAAAGGCCACAAACCAGUA |
| siRNA 5428 | 5428 | ACUGGUUUGUGGCCUUUUA | 10918 | UAAAAGGCCACAAACCAGU |
| siRNA 5429 | 5429 | CUGGUUUGUGGCCUUUUAC | 10919 | GUAAAAGGCCACAAACCAG |
| siRNA 5430 | 5430 | UGGUUUGUGGCCUUUUACU | 10920 | AGUAAAAGGCCACAAACCA |
| siRNA 5431 | 5431 | GGUUUGUGGCCUUUUACUG | 10921 | CAGUAAAAGGCCACAAACC |
| siRNA 5432 | 5432 | GUUUGUGGCCUUUUACUGU | 10922 | ACAGUAAAAGGCCACAAAC |
| siRNA 5433 | 5433 | UUUGUGGCCUUUUACUGUG | 10923 | CACAGUAAAAGGCCACAAA |
| siRNA 5434 | 5434 | UUGUGGCCUUUUACUGUGC | 10924 | GCACAGUAAAAGGCCACAA |
| siRNA 5435 | 5435 | UGUGGCCUUUUACUGUGCU | 10925 | AGCACAGUAAAAGGCCACA |
| siRNA 5436 | 5436 | GUGGCCUUUUACUGUGCUU | 10926 | AAGCACAGUAAAAGGCCAC |
| siRNA 5437 | 5437 | UGGCCUUUUACUGUGCUUU | 10927 | AAAGCACAGUAAAAGGCCA |
| siRNA 5438 | 5438 | GGCCUUUUACUGUGCUUUG | 10928 | CAAAGCACAGUAAAAGGCC |
| siRNA 5439 | 5439 | GCCUUUUACUGUGCUUUGU | 10929 | ACAAAGCACAGUAAAAGGC |
| siRNA 5440 | 5440 | CCUUUUACUGUGCUUUGUA | 10930 | UACAAAGCACAGUAAAAGG |
| siRNA 5441 | 5441 | CUUUUACUGUGCUUUGUAU | 10931 | AUACAAAGCACAGUAAAAG |
| siRNA 5442 | 5442 | UUUUACUGUGCUUUGUAUC | 10932 | GAUACAAAGCACAGUAAAA |
| siRNA 5443 | 5443 | UUUACUGUGCUUUGUAUCA | 10933 | UGAUACAAAGCACAGUAAA |
| siRNA 5444 | 5444 | UUACUGUGCUUUGUAUCAG | 10934 | CUGAUACAAAGCACAGUAA |
| siRNA 5445 | 5445 | UACUGUGCUUUGUAUCAGA | 10935 | UCUGAUACAAAGCACAGUA |
| siRNA 5446 | 5446 | ACUGUGCUUUGUAUCAGAG | 10936 | CUCUGAUACAAAGCACAGU |
| siRNA 5447 | 5447 | CUGUGCUUUGUAUCAGAGU | 10937 | ACUCUGAUACAAAGCACAG |
| siRNA 5448 | 5448 | UGUGCUUUGUAUCAGAGUU | 10938 | AACUCUGAUACAAAGCACA |
| siRNA 5449 | 5449 | GUGCUUUGUAUCAGAGUUC | 10939 | GAACUCUGAUACAAAGCAC |
| siRNA 5450 | 5450 | UGCUUUGUAUCAGAGUUCU | 10940 | AGAACUCUGAUACAAAGCA |
| siRNA 5451 | 5451 | GCUUUGUAUCAGAGUUCUU | 10941 | AAGAACUCUGAUACAAAGC |
| siRNA 5452 | 5452 | CUUUGUAUCAGAGUUCUUA | 10942 | UAAGAACUCUGAUACAAAG |
| siRNA 5453 | 5453 | UUUGUAUCAGAGUUCUUAA | 10943 | UUAAGAACUCUGAUACAAA |
| siRNA 5454 | 5454 | UUGUAUCAGAGUUCUUAAC | 10944 | GUUAAGAACUCUGAUACAA |
| siRNA 5455 | 5455 | UGUAUCAGAGUUCUUAACA | 10945 | UGUUAAGAACUCUGAUACA |
| siRNA 5456 | 5456 | GUAUCAGAGUUCUUAACAA | 10946 | UUGUUAAGAACUCUGAUAC |
| siRNA 5457 | 5457 | UAUCAGAGUUCUUAACAAG | 10947 | CUUGUUAAGAACUCUGAUA |
| siRNA 5458 | 5458 | AUCAGAGUUCUUAACAAGA | 10948 | UCUUGUUAAGAACUCUGAU |
| siRNA 5459 | 5459 | UCAGAGUUCUUAACAAGAU | 10949 | AUCUUGUUAAGAACUCUGA |
| siRNA 5460 | 5460 | CAGAGUUCUUAACAAGAUU | 10950 | AAUCUUGUUAAGAACUCUG |
| siRNA 5461 | 5461 | AGAGUUCUUAACAAGAUUA | 10951 | UAAUCUUGUUAAGAACUCU |
| siRNA 5462 | 5462 | GAGUUCUUAACAAGAUUAA | 10952 | UUAAUCUUGUUAAGAACUC |
| siRNA 5463 | 5463 | AGUUCUUAACAAGAUUAAU | 10953 | AUUAAUCUUGUUAAGAACU |
| siRNA 5464 | 5464 | GUUCUUAACAAGAUUAAUA | 10954 | UAUUAAUCUUGUUAAGAAC |
| siRNA 5465 | 5465 | UUCUUAACAAGAUUAAUAA | 10955 | UUAUUAAUCUUGUUAAGAA |
| siRNA 5466 | 5466 | UCUUAACAAGAUUAAUAAA | 10956 | UUUAUUAAUCUUGUUAAGA |
| siRNA 5467 | 5467 | CUUAACAAGAUUAAUAAAU | 10957 | AUUUAUUAAUCUUGUUAAG |
| siRNA 5468 | 5468 | UUAACAAGAUUAAUAAAUC | 10958 | GAUUUAUUAAUCUUGUUAA |
| siRNA 5469 | 5469 | UAACAAGAUUAAUAAAUCA | 10959 | UGAUUUAUUAAUCUUGUUA |
| siRNA 5470 | 5470 | AACAAGAUUAAUAAAUCAC | 10960 | GUGAUUUAUUAAUCUUGUU |
| siRNA 5471 | 5471 | ACAAGAUUAAUAAAUCACC | 10961 | GGUGAUUUAUUAAUCUUGU |
| siRNA 5472 | 5472 | CAAGAUUAAUAAAUCACCC | 10962 | GGGUGAUUUAUUAAUCUUG |
| siRNA 5473 | 5473 | AAGAUUAAUAAAUCACCCC | 10963 | GGGGUGAUUUAUUAAUCUU |
| siRNA 5474 | 5474 | AGAUUAAUAAAUCACCCCA | 10964 | UGGGGUGAUUUAUUAAUCU |
| siRNA 5475 | 5475 | GAUUAAUAAAUCACCCCAG | 10965 | CUGGGGUGAUUUAUUAAUC |
| siRNA 5476 | 5476 | AUUAAUAAAUCACCCCAGU | 10966 | ACUGGGGUGAUUUAUUAAU |
| siRNA 5477 | 5477 | UUAAUAAAUCACCCCAGUC | 10967 | GACUGGGGUGAUUUAUUAA |
| siRNA 5478 | 5478 | UAAUAAAUCACCCCAGUCU | 10968 | AGACUGGGGUGAUUUAUUA |
| siRNA 5479 | 5479 | AAUAAAUCACCCCAGUCUU | 10969 | AAGACUGGGGUGAUUUAUU |
| siRNA 5480 | 5480 | AUAAAUCACCCCAGUCUUA | 10970 | UAAGACUGGGGUGAUUUAU |
| siRNA 5481 | 5481 | UAAAUCACCCCAGUCUUAA | 10971 | UUAAGACUGGGGUGAUUUA |
| siRNA 5482 | 5482 | AAAUCACCCCAGUCUUAAU | 10972 | AUUAAGACUGGGGUGAUUU |
| siRNA 5483 | 5483 | AAUCACCCCAGUCUUAAUU | 10973 | AAUUAAGACUGGGGUGAUU |
| siRNA 5484 | 5484 | AUCACCCCAGUCUUAAUUU | 10974 | AAAUUAAGACUGGGGUGAU |
| siRNA 5485 | 5485 | UCACCCCAGUCUUAAUUUU | 10975 | AAAAUUAAGACUGGGGUGA |
| siRNA 5486 | 5486 | CACCCCAGUCUUAAUUUUU | 10976 | AAAAAUUAAGACUGGGGUG |
| siRNA 5487 | 5487 | ACCCCAGUCUUAAUUUUUA | 10977 | UAAAAAUUAAGACUGGGGU |
| siRNA 5488 | 5488 | CCCCAGUCUUAAUUUUUAA | 10978 | UUAAAAAUUAAGACUGGGG |
| siRNA 5489 | 5489 | CCCAGUCUUAAUUUUUAAA | 10979 | UUUAAAAAUUAAGACUGGG |
| siRNA 5490 | 5490 | CCAGUCUUAAUUUUUAAAA | 10980 | UUUUAAAAAUUAAGACUGG |

| SEQ ID | |
|---|---|
| NO: | 5′ to 3′ Sequence |
| 11253 | AGAAGCGGGCCAGCGCCGCCGGGAAAGGAGGTCGCCGCCCGGGGTCGCCCGGGCTTGCGGG |
| | TCGCCGCGCTTCCTCTGCGGGCGCTGCGGGAGGCGAGGGCCGCCTCGGCGGAGGAAGCCGA |
| | CCCCGGGGTCTCCGCCAGCCCGCCGGCGCCGCCTCCGAGCCGCCTCGGCCGTAGCCTCGGCC |
| | CGGGCGGGAGGCGGCGGCCGCCGGCGAGGCGAGGCGGCCCGCGCCCTGCCTGTCAGGCCAC |
| | CGGCCCGGCCCGCGGGCTGCCCCCGATGCGGAGGCGCTGTCGCCGGGGCCATGCAGCAGGC |
| | GCCGCAGCCTTACGAGTTCTTCAGCGAGGAGAACAGTCCGAAATGGCGGGGACTGTTGGTCT |
| | CGGCCCTGCGGAAGGTTCAGGAACAAGTGCATCCCACTCTCTCAGCTAATGAAGAGTCTCTC |
| | TATTATATTGAAGAGCTGATTTTTCAGCTGCTTAATAAATTATGCATGGCCCAGCCAAGGACT |
| | GTTCAAGATGTAGAGGAGCGAGTTCAGAAGACCTTTCCTCACCCAATTGATAAATGGGCCAT |
| | TGCTGATGCACAATCTGCTATAGAAAAACGAAAACGAAGAAATCCTCTTTTACTGCCTGTGG |
| | ACAAAATCCATCCTTCGTTGAAGGAAGTATTAGGGTACAAAGTGGACTACCATGTATCCCTA |
| | TATATTGTGGCTGTACTAGAGTATATCTCAGCTGATATTTTAAAATTGGCTGGTAATTATGTT |
| | TTTAATATCCGGCATTATGAAATATCTCAGCAGGACATTAAAGTGTCAATGTGTGCGGATAA |
| | GGTTTTGATGGACATGTTTGATCAGGATGACATAGGTTTGGTTTCTCTCTGTGAAGATGAACC |
| | TAGTTCTTCTGGTGAATTAAACTACTATGATCTTGTCAGAACTGAAATCGCAGAAGAAAGAC |
| | AGTATCTACGGGAATTAAATATGATCATAAAAGTGTTTCGAGAAGCCTTTCTTTCTGATAGA |
| | AAGCTGTTTAAACCTTCTGATATCGAAAAGATTTTTAGTAACATTTCAGATATACATGAATTG |
| | ACTGTGAAACTTTTAGGTTTGATTGAAGACACAGTTGAAATGACTGATGAAAGCAGTCCTCA |
| | TCCCTTAGCTGGCAGCTGTTTTGAAGATTTGGCAGAAGAGCAAGCATTTGATCCTTATGAAA |
| | CATTATCACAGGACATTCTTTCACCAGAGTTTCATGAACATTTCAATAAATTGATGGCCAGA |
| | CCTGCAGTTGCTCTACACTTTCAGTCCATTGCTGATGGTTTTAAAGAGGCAGTTCGTTATGTC |
| | CTTCCACGTCTTATGCTGGTGCCAGTGTATCACTGTTGGCACTACTTTGAGTTACTAAAGCAA |
| | TTGAAAGCATGTAGTGAAGAACAAGAAGACAGAGAATGTTTGAACCAAGCTATTACTGCTC |
| | TCATGAATCTCCAAGGTAGCATGGACCGAATTTACAAGCAGTATTCACCTAGACGTCGACCT |
| | GGAGATCCTGTTTGCCCTTTTTATAGTCACCAATTAAGAAGCAAACACCTGGCTATCAAAAA |
| | AATGAATGAAATTCAGAAAAATATCGATGGATGGGAAGGCAAAGATATTGGACAGTGTTGT |
| | AATGAATTCATTATGGAGGGACCATTGACAAGAATCGGTGCCAAACATGAACGGCATATTTT |
| | TCTGTTTGATGGCTTAATGATCAGTTGTAAACCTAATCATGGCCAGACTCGGCTTCCAGGTTA |
| | CAGTAGTGCAGAATACAGGTTAAAAGAAAAATTTGTCATGAGGAAAATACAAATTTGTGAT |
| | AAAGAAGATACTTGTGAGCACAAGCATGCATTTGAATTAGTATCCAAAGATGAGAACAGCA |
| | TAATATTTGCTGCTAAGTCTGCTGAAGAAAAAAACAACTGGATGGCAGCCCTTATTTCTCTTC |
| | ATTATCGTAGTACTCTAGATCGAATGTTAGATTCAGTATTATTGAAAGAAGAAAATGAGCAA |
| | CCACTGAGATTACCAAGTCCTGAAGTATATCGTTTTGTAGTAAAAGACTCTGAGGAAAACAT |
| | TGTTTTTGAAGACAACTTGCAAAGTAGAAGTGGCATCCCCATTATTAAAGGAGGAACTGTAG |
| | TGAAATTAATTGAAAGGTTAACATATCATATGTATGCAGATCCCAATTTTGTTCGTACTTTTC |
| | TTACCACATATCGTTCATTTTGTAAACCACAGGAATTGCTGAGCTTACTGATTGAACGGTTTG |
| | AAATTCCAGAGCCAGAACCTACTGACGCAGACAAATTGGCAATAGAGAAAGGCGAGCAGCC |
| | AATCAGTGCAGACCTTAAAAGATTTCGCAAGGAATATGTCCAACCAGTACAACTTAGGATCT |
| | TAAATGTATTTCGGCATTGGGTTGAACATCATTTTTATGACTTTGAAAGAGACTTGGAATTGC |
| | TTGAAAGACTAGAATCCTTCATTTCAAGTGTAAGAGGGAAAGCTATGAAAAAATGGGTAGA |
| | GTCAATTGCTAAGATCATCAGGAGGAAGAAGCAAGCTCAGGCAAACGGAGTAAGCCATAAT |
| | ATTACCTTTGAAAGTCCACCTCCACCAATTGAATGGCATATCAGCAAACCAGGACAGTTTGA |
| | AACATTTGATCTCATGACACTTCATCCAATAGAAATTGCACGTCAGCTGACACTTTTGGAGTC |
| | TGATCTTTACAGGAAAGTTCAACCGTCTGAACTTGTAGGGAGTGTGTGGACCAAAGAAGATA |
| | AAGAAATAAATTCTCCAAATTTATTAAAAATGATTCGCCATACCACAAATCTCACCCTCTGG |
| | TTTGAAAAATGCATTGTGGAAGCAGAAAATTTTGAAGAACGGGTGGCAGTACTAAGTAGAA |
| | TTATAGAAATTCTGCAAGTTTTTCAAGATTTGAATAATTTCAATGGCGTATTGGAGATAGTCA |
| | GTGCAGTAAATTCAGTGTCAGTATACAGACTAGACCATACCTTTGAGGCACTGCAGGAAAGG |
| | AAAAGGAAAATTTTGGACGAAGCTGTGGAATTAAGTCAAGATCACTTTAAAAAATACCTAG |
| | TAAAACTTAAGTCAATCAATCCACCTTGTGTGCCTTTTTTTGGAATATATTTAACAAATATTC |
| | TGAAGACCGAAGAAGGGAATAATGATTTTTTAAAAAAGAAAGGGAAAGATTTAATCAATTT |
| | CAGTAAGAGGAGGAAAGTAGCTGAAATTACTGGAGAAATTCAGCAGTATCAGAATCAGCCT |
| | TACTGTTTACGGATAGAACCAGATATGAGGAGATTCTTTGAAAACCTTAACCCCATGGGAAG |
| | TGCATCTGAAAAAGAGTTTACAGATTATTTGTTCAACAAGTCACTAGAAATTGAACCTCGAA |
| | ACTGCAAACAGCCACCTCGATTTCCTAGGAAATCAACTTTTTCCTTAAAATCTCCTGGAATAA |
| | GGCCTAACACAGGCCGACATGGCTCTACCTCAGGTACTTTACGAGGTCACCCAACACCATTA |
| | GAAAGAGAACCATGTAAAATAAGCTTTAGTCGGATTGCTGAAACTGAGCTGGAATCAACAG |
| | TGTCAGCACCAACCTCTCCAAATACACCATCTACTCCACCAGTATCTGCTTCTTCAGACCTTA |
| | GTGTATTTTTAGATGTGGATCTCAACAGCTCCTGTGGCAGCAATAGCATCTTTGCTCCAGTGC |
| | TTTTGCCACATTCAAAGTCTTTCTTTAGTTCATGTGGTAGTTTACATAAACTAAGTGAAGAGC |
| | CCCTGATTCCTCCTCCTCTTCCTCCTCGAAAAAAGTTTGATCATGATGCTTCAAATTCCAAGG |
| | GAAATATGAAATCTGATGATGATCCTCCTGCTATTCCACCGAGACAGCCTCCTCCTCCAAAG |
| | GTAAAACCCAGAGTTCCTGTTCCTACTGGTGCATTTGATGGGCCTCTGCATAGTCCACCTCCG |
| | CCACCACCAAGAGATCCTCTTCCTGATACCCCTCCACCAGTTCCCCTTCGGCCTCCAGAACAC |
| | TTTATAAACTGTCCATTTAATCTTCAGCCACCTCCACTGGGGCATCTTCACAGAGATTCAGAC |
| | TGGCTCAGAGACATTAGTACGTGTCCAAATTCGCCAAGCACTCCTCCTAGCACACCCTCTCC |
| | AAGGGTACCGCGTCGATGCTATGTGCTCAGTTCTAGTCAGAATAATCTTGCTCATCCTCCAGC |
| | TCCCCCTGTTCCACCAAGGCAGAATTCAAGCCCTCATCTGCCAAAACTGCCACCAAAGACTT |
| | ACAAACGGGAGCTTTCGCACCCCCCATTGTACAGACTGCCTTTGCTAGAAAATGCAGAAACT |
| | CCCCAATGACCTTAGCCATATGTAGTCATTGACACTGGAATGGTATTTGTAAAGTTTTTTTTT |
| | TAATTTATTCAAAAAAAGACATAGTATTTTAGTACTTTTTACAAATAATGCTCTTACAAAAAT |
| | GCTACTGATCAAATAAGCTTTTAAGAATTGGAAAAATAAAAATACAAAAGTCCTTCACCATT |
| | ATCCTCAGGTGATCAGTAGCATTGCCTTGTCTTGGATCCTCAGTGCTGCCAAAAGGCCAGTA |
| | TAAAGAATTTATATTTGCACTGTAAACTCTGCAAAAATATGGTTTAAAGTGACATGATTGCA |
| | CTGAAAAGGGATAGTGCTTTTGTGAAATTTTTCAAATTTGAGTAATAGATGCCTTTTTAAGGC |
| | AGTGAATTTACACAAATATGGGAGGGGTATATGGTGTTACTGATTTTTAAACCTCTTTGACC |
| | ATCTTCTAGTTTTTACTTCTAGTTTTTACATCTAGGAGAATTGTGAATAACACCAACTGTTCTT |
| | AAACTCTTTAATGCCATGTCTTAAATGCCGGTATTTGCTGCTGAAGACAAAAATGAAAAGTA |
| | GGATGAAAATAATAGAATGGCACTGTAAGTGTTTATTATTTTGTCAAAATGTAAACAAAGAC |
| | TACATAACCCAATGATGGAGGGAAAAAGGGCATGTATCTCATTCAGATGTGCCTTTTGTTTT |
| | TGCAGACTATGACGTCTTTAGCTAATGAATTGCCTATTGTTATGGAAAACAGTTAATATGCC |
| | ATGTATGTACAGTTTTGTTTATATTGTATATTTAAAGATACTGCTAATAACCTATATAAATTT |
| | AAGTGACTTGAGGCCTATAATACAATCTGCTACTTTACTAATTCATAAATTCAAAAAAAATT |
| | CTATGGCATAGGAACCAACTGCCTTGCCTTCAAGACCTAGTAACTTTCTCTATAAATCTCGTG |
| | TTAACTGAAATTTTTTTTAAATATATTTTTTAGATTGGTAATATTTAAACCAGCAAATACTTA |
| | AAGCTTTATTAAACATTTTAATCAGATAAGTGAGTAAAGCTTTTATTTGCCATTTGGATGCCT |
| | TCGTTCAAAGTGATAGAGTGTTTTGCTGATAGTGCTGTAGCAGCAGTTGTAAAGTAGCCAAA |
| | AGCCACGTTGTTTATTTACTGGTTTGTGGCCTTTTACTGTGCTTTGTATCAGAGTTCTTAACAA |
| | GATTAATAAATCACCCCAGTCTTAATTTTTAAAA |

What is claimed is:

1. A composition comprising an oligonucleotide that targets SOS2 and when administered to a subject in an effective amount increases an estimated glomerular filtration rate, or decreases a creatinine, blood urea nitrogen, proteinuria microalbuminuria measurement, or urine albumin creatinine ratio.

2. The composition of claim 1, wherein the estimated glomerular filtration rate is increased, or the creatinine, blood urea nitrogen, proteinuria, microalbuminuria measurement or urine albumin creatinine ratio is decreased, by about 10% or more, as compared to prior to administration.

3. A composition comprising an oligonucleotide that targets SOS2 and when administered to a subject in an effective amount decreases a blood urate measurement.

4. The composition of claim 3, wherein the blood urate measurement is decreased by about 10% or more, as compared to prior to administration.

5. A composition comprising an oligonucleotide that targets SOS2 and when administered to a subject in an effective amount decreases a systolic blood pressure measurement, a diastolic blood pressure measurement, a mean arterial pressure, or a pulse pressure.

6. The composition of claim 5, wherein the systolic blood pressure measurement, diastolic blood pressure measurement, mean arterial pressure, or pulse pressure is decreased by about 10% or more, as compared to prior to administration.

7. A composition comprising an oligonucleotide that targets SOS2 and when administered to a subject in an effective amount decreases an intraocular pressure measurement, cup-disc ratio, optic nerve cupping, RPE pigmentation and reflectivity, drusen, Macular hemorrhage, choroidal neovascularization, edema, microaneurysms, intraretinal hemorrhage, macular ischemia, neovascularization, vitreous hemorrhage, or traction retinal detachment or increases a RNFL thickness or retinal thickness.

8. The composition of claim 7, wherein the intraocular pressure measurement, cup-disc ratio, optic nerve cupping, RPE pigmentation and reflectivity, drusen, Macular hemorrhage, choroidal neovascularization, edema, microaneurysms, intraretinal hemorrhage, macular ischemia, neovascularization, vitreous hemorrhage, or traction retinal detachment is decreased or the RNFL thickness or retinal thickness is increased by about 10% or more, as compared to prior to administration.

9. A composition comprising an oligonucleotide that targets SOS2 and when administered to a subject in an effective amount decreases a body mass index (BMI) measurement, a body weight measurement, a waist circumference measurement, a hip circumference measurement, a waist-hip ratio (WHR), a body fat percentage measurement, a hemoglobin A1C measurement, a blood glucose measurement, a glucose tolerance measurement, an insulin sensitivity measurement, a blood triglyceride measurement, or a non-HDL cholesterol measurement.

10. The composition of claim 9, wherein the body mass index (BMI) measurement, the body weight measurement, the waist circumference measurement, the hip circumference measurement, the waist-hip ratio (WHR), the body fat percentage measurement, the hemoglobin A1C measurement, the blood glucose measurement, the glucose tolerance measurement, the insulin sensitivity measurement, the blood triglyceride measurement, or the non-HDL cholesterol measurement is decreased by about 10% or more, as compared to prior to administration.

11. A composition comprising an oligonucleotide that targets SOS2 and when administered to a subject in an effective amount decreases an alanine aminotransferase, aspartate aminotransferase, liver fat percentage measurement, liver fibrosis score, NAFLD activity score, or blood gamma-glutamyl transferase measurement.

12. The composition of claim 11, wherein the alanine aminotransferase, aspartate aminotransferase, liver fat percentage measurement, liver fibrosis score, NAFLD activity score, or blood gamma-glutamyl transferase measurement is decreased by about 10% or more, as compared to prior to administration.

13. The composition of any one of claims 1-12, wherein the oligonucleotide comprises a small interfering RNA (siRNA) comprising a sense strand and an antisense strand.

14. The composition of claim 13, wherein the sense strand is 12-30 nucleosides in length.

15. The composition of claim 13, wherein the sense strand comprises the sequence of any one of SEQ ID NOs: 1-5490, or a nucleic acid sequence thereof having 1 or 2 nucleoside substitutions, additions, or deletions.

16. The composition of claim 15, wherein the sense strand comprises the sequence of any one of SEQ ID NOs: 1-5490.

17. The composition of claim 13, wherein the antisense strand is 12-30 nucleosides in length.

18. The composition of claim 13, wherein the antisense strand comprises the sequence of any one of SEQ ID NOs: 5491-10980, or a nucleic acid sequence thereof having 1 or 2 nucleoside substitutions, additions, or deletions.

19. The composition of claim 18, wherein the antisense strand comprises the sequence of any one of SEQ ID NOs: 5491-10980.

20. The composition of claim 13, wherein the sense or antisense strand comprises a sense or antisense sequence of an siRNA of any one of Tables 35-35, or a nucleic acid sequence thereof having 1 or 2 nucleoside substitutions, additions, or deletions.

21. The composition of claim 20, wherein the sense or antisense strand comprises a sense or antisense sequence of an siRNA of any one of Tables 15-25.

22. The composition of claim 13, wherein any one of the following is true with regard to the sense strand:
(a) all purines comprise 2′ fluoro modified purines, and all pyrimidines comprise a mixture of 2′ fluoro and 2′-O-methyl modified pyrimidines;
(b) all purines comprise 2′-O-methyl modified purines, and all pyrimidines comprise a mixture of 2′ fluoro and 2′-O-methyl modified pyrimidines;
(c) all purines comprise 2′ fluoro modified purines, and all pyrimidines comprise 2′-O-methyl modified pyrimidines;
(d) all pyrimidines comprise 2′ fluoro modified pyrimidines, and all purines comprise a mixture of 2′ fluoro and 2′-O-methyl modified purines;
(e) all pyrimidines comprise 2′-O-methyl modified pyrimidines, and all purines comprise a mixture of 2′ fluoro and 2′-O-methyl modified purines; or
(f) all pyrimidines comprise 2′ fluoro modified pyrimidines, and all purines comprise 2′-O-methyl modified purines.

23. The composition of claim 13, wherein any one of the following is true with regard to the antisense strand:
(a) all purines comprise 2′ fluoro modified purines, and all pyrimidines comprise a mixture of 2′ fluoro and 2′-O-methyl modified pyrimidines;
(b) all purines comprise 2′-O-methyl modified purines, and all pyrimidines comprise a mixture of 2′ fluoro and 2′-O-methyl modified pyrimidines;
(c) all purines comprise 2′-O-methyl modified purines, and all pyrimidines comprise 2′ fluoro modified pyrimidines;
(d) all pyrimidines comprise 2′ fluoro modified pyrimidines, and all purines comprise a mixture of 2′ fluoro and 2′-O-methyl modified purines;
(e) all pyrimidines comprise 2′-O-methyl modified pyrimidines, and all purines comprise a mixture of 2′ fluoro and 2′-O-methyl modified purines; or
(f) all pyrimidines comprise 2′-O-methyl modified pyrimidines, and all purines comprise 2′ fluoro modified purines.

24. A composition comprising an oligonucleotide that inhibits the expression of SOS2 wherein the oligonucleotide comprises an siRNA comprising a sense strand and an antisense strand, each strand is independently about 12-30 nucleosides in length, and at least one of the sense strand and the antisense strand comprises a nucleoside sequence comprising about 12-30 contiguous nucleosides of SEQ ID NO: 11253.

25. The composition of any one of claims 1-12, wherein the oligonucleotide comprises an antisense oligonucleotide (ASO).

26. A composition comprising an oligonucleotide that inhibits the expression of SOS2 wherein the oligonucleotide comprises an ASO that is complementary to a nucleoside sequence comprising about 12-30 contiguous nucleosides of SEQ ID NO: 11253.

27. The composition of claim 25, wherein the ASO is 12-30 nucleosides in length.

28. The composition of any one of claims 1-12, wherein the oligonucleotide comprises a modified internucleoside linkage.

29. The composition of claim 28, wherein the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

30. The composition of claim 28, wherein the modified internucleoside linkage comprises one or more phosphorothioate linkages.

31. The composition of any one of claims 1-12, wherein the oligonucleotide comprises 2, 3, 4, 5, 6, 7, 8, 9, 10, 11, 12, 13, 14, 15, 16, 17, 18, 19, or 20 modified internucleoside linkages.

32. The composition of any one of claims 1-12, wherein the oligonucleotide comprises a modified nucleoside.

33. The composition of claim 32, wherein the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

34. The composition of claim 32, wherein the modified nucleoside comprises a LNA.

35. The composition of claim 32, wherein the modified nucleoside comprises a 2′,4′ constrained ethyl nucleic acid.

36. The composition of claim 32, wherein the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof.

37. The composition of claim 32, wherein the modified nucleoside comprises one or more 2′fluoro modified nucleosides.

38. The composition of claim 32, wherein the modified nucleoside comprises a 2′ O-alkyl modified nucleoside.

39. The composition of claim 32, wherein the oligonucleotide comprises a lipid attached at a 3′ or 5′ terminus of the oligonucleotide.

40. The composition of claim 39, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof.

41. The composition of claim 39, wherein the lipid comprises a 5′ hydrophobic moiety comprising any one of the following structures: 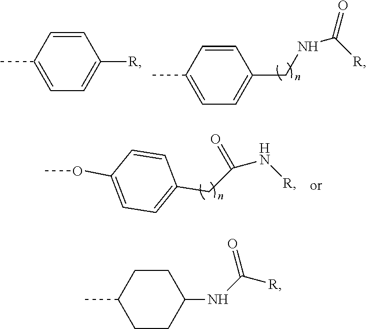
wherein the dotted line indicates a covalent connection to the end of the 5′ end of the sense strand, n is 1-3, and R is an alkyl group containing 4-18 carbons.

42. The composition of any one of claims 1-12, wherein the oligonucleotide comprises 2, 3, 4, 5, 6, 7, 8, 9, 10, 11, 12, 13, 14, 15, 16, 17, 18, 19, 20, or 21 modified nucleosides.

43. The composition of any one of claims 1-12, wherein the oligonucleotide comprises an N-acetylgalactosamine (GalNAc) ligand, an arginine-glycine-aspartic acid (RGD) peptide, or a cholesterol ligand.

44. The composition of claim 43, wherein the oligonucleotide comprises a GalNAc ligand.

45. The composition of claim 44, wherein the GalNac ligand comprises 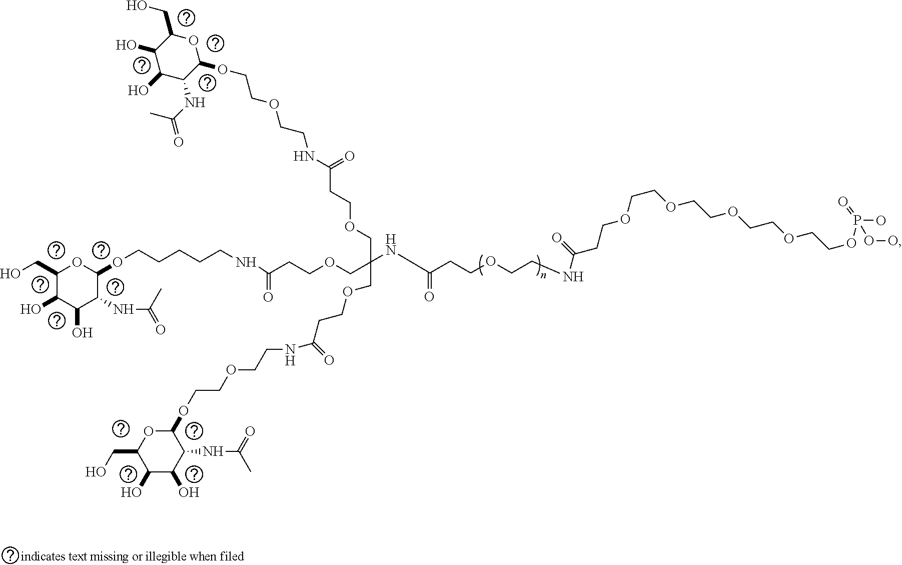
wherein n is 1 or 2, and J is the oligonucleotide.

46. A method of treating chronic kidney disease, diabetic nephropathy, gout, hyperuricemia, hypertension, cerebrovascular disease, type 2 diabetes, metabolic syndrome, obesity, hyperlipidemia, hypertriglyceridemia, glaucoma, ocular hypertension, retinal diseases, age-related macular degeneration, choroidal neovascularization, geographic atrophy, diabetic retinopathy, non-alcoholic fatty liver disease, fibrotic liver disease, liver fibrosis, cirrhosis, or hair loss in a subject in need thereof comprising administering to the subject a composition according to any one of claims 1-12.

47. A composition comprising an oligonucleotide that targets SOS2, wherein the oligonucleotide comprises a small interfering RNA (siRNA) comprising a sense strand and an antisense strand; and
wherein the sense strand comprises the nucleoside sequence of any one of SEQ ID NOs: 1-5490 or a nucleic acid sequence thereof having 1 or 2 nucleoside substitutions, additions, or deletions;
or wherein the antisense strand comprises the nucleoside sequence of any one of SEQ ID NOs: 5491-10980 or a nucleic acid sequence thereof having 1 or 2 nucleoside substitutions, additions, or deletions.

48. A composition comprising a compound represented by Formula (I) or (II): 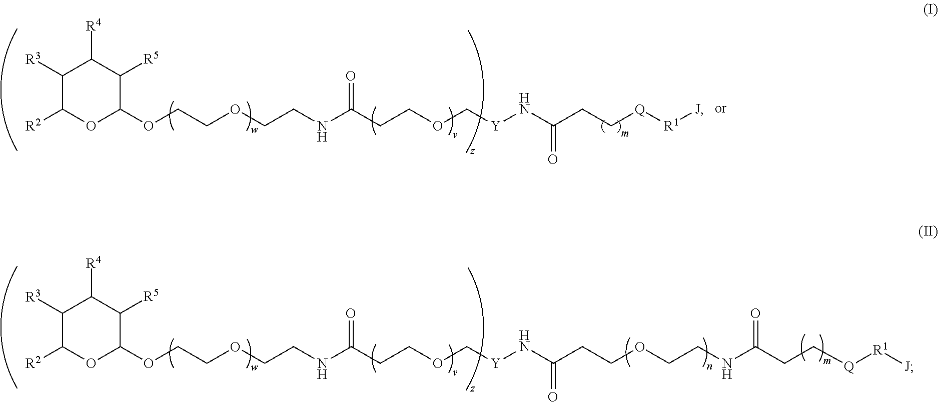
or a salt thereof, wherein
J is an oligonucleotide targeting SOS comprising a small interfering RNA (siRNA) comprising a sense strand and an antisense strand;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
if z is 3, Y is C
if z is 2, Y is CR⁶, or
if z is 1, Y is C(R⁶)₂;
Q is selected from:
C₃₋₁₀ carbocycle optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, and C₁₋₆ alkyl, wherein the C₁₋₆ alkyl, is optionally substituted with one or more substituents independently selected from halogen, —CN, —OH, —SH, —NO₂, and —NH₂;
R¹ is a linker selected from:
—O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from:
C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
R³ and R⁴ are each independently selected from:
—OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from:
—OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
hydrogen;
halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
C₁₋₆ alkyl optionally substituted with one or more substituents independently selected from halogen, —CN, —NO₂, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁷ is independently selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl.

49. A composition comprising an oligonucleotide that inhibits the expression of SOS2 wherein the oligonucleotide comprises an siRNA comprising a sense strand and an antisense strand, wherein the oligonucleotide comprises a 5′ hydrophobic moiety comprising any one of the following structures: 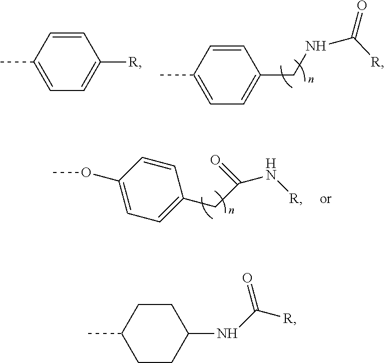
wherein the dotted line indicates a covalent connection to the end of the 5′ end of the sense strand, n is 1-3, and R is an alkyl group containing 4-18 carbons.
